(19) United States
(12) Patent Application Publication
(10) Pub. No.: US 2023/0114728 A1
(43) Pub. Date: Apr. 13, 2023

(54) PENICILLIN-BINDING PROTEIN INHIBITORS

(71) Applicant: Venatorx Pharmaceuticals, Inc., Malvem, PA (US)

(72) Inventors: Christopher J. BURNS, Malvern, PA (US); Denis DAIGLE, Street, MD (US); Guo-Hua CHU, Exton, PA (US); Jodie HAMRICK, New Holland, PA (US); Steven A. BOYD, Chester Springs, PA (US); Allison L. ZULLI, Chesterbrook, PA (US); Stephen M. CONDON, Glenmoore, PA (US); Cullen L. MYERS, Phoenixville, PA (US); Zhenrong XU, Chalfont, PA (US); Tsuyoshi Uehara, Berwyn, PA (US); Nathan LINE, Royersford, PA (US)

(21) Appl. No.: 17/779,804

(22) PCT Filed: Sep. 24, 2020
(86) PCT No.: PCT/US2020/052439

§ 371 Date: May 25, 2022

Related U.S. Application Data
(60) Provisional application No. 62/940,538, filed on Nov. 26, 2019.

(51) Int. Cl. C07F 5/02; A61P 31/04; A61K 47/20

(52) U.S. Cl. CPC C07F 5/02; A61P 31/04; A61K 47/20

(57) ABSTRACT

Described herein are certain boron-containing compounds, compositions, preparations and their use as modulators of the transpeptidase function of bacterial penicillin-binding proteins and as antibacterial agents. In some embodiments, the compounds described herein inhibit penicillin-binding proteins. In certain embodiments, the compounds described herein are useful in the treatment of bacterial infections.

### CROSS-REFERENCE

This application claims the benefit of U.S. Provisional Application Ser. No. 62/940,538 filed Nov. 26, 2019, which is hereby incorporated by reference in its entirety.

### STATEMENT AS TO FEDERALLY SPONSORED RESEARCH

This invention was made with government support under SBIR Grant number 5R43AI094827 by the National Institutes of Health (NIH), 1R01AI141239 by the National Institutes of Health (NIH), Federal Award 6 IDSEP16030-01-02, subaward 4500002377, awarded by the Health and Human Services Office of the Assistant Secretary for Preparedness and Response (HHS/ASPR) under the CARB-X Pass Through Entity, Federal Award 6 IDSEP16030-01-02, subaward 4500003206, awarded by the Health and Human Services Office of the Assistant Secretary for Preparedness and Response (HHS/ASPR) under the CARB-X Pass Through Entity, and contract HDTRA117C0070, awarded by the Defense Threat Reduction Agency (DTRA) of the Department of Defense. The government has certain rights in the invention.

### BACKGROUND OF THE INVENTION

Antibiotics are the most effective drugs for curing bacteria-related infectious diseases clinically. They are incredibly valuable therapeutic options that are currently losing efficacy due to the evolution and spread of drug resistance genes, leading to multidrug resistance bacterial organisms. Among the different classes of antibiotics, the penicillin-binding protein-targeting beta-lactams (e.g. penicillins, cephalosporins, and carbapenems) are the most widely used antibiotic class because they have a strong bactericidal effect and low associated toxicity.

Penicillin Binding Proteins (PBPs) are a family of essential bacterial enzymes involved in the synthesis of peptidoglycan, the major structural polymer found in the bacterial cell wall. Beta-lactam antibiotics bind with high affinity to PBPs and inhibit their transpeptidase function, resulting in disruption of peptidoglycan cell wall synthesis and rapid cell lysis of actively dividing bacteria. As there are no close mammalian homologues to PBPs, and beta-lactams are well-regarded for their safety and efficacy, PBPs represent an ideal target for antibacterials.

### SUMMARY OF THE INVENTION

Described herein are compounds that inhibit the activity of penicillin-binding proteins, the bacterial enzyme class targeted by the beta lactam antibiotics and do provide significant antibacterial activity in vitro.

Provided herein are compounds of Formula (IIa) or (IIb), or a pharmaceutically acceptable salt, solvate, stereoisomer, tautomer, N-oxide, dimer, or trimer thereof:

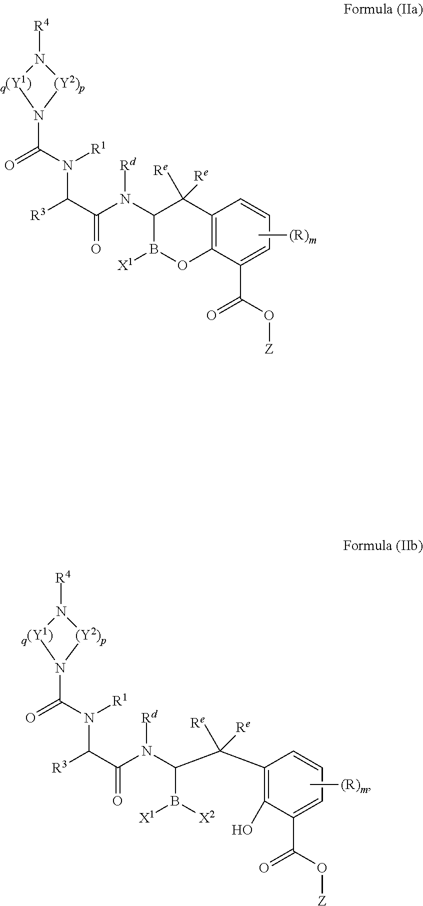

wherein:
R¹ is hydrogen or C₁-C₆ alkyl;
R³ is

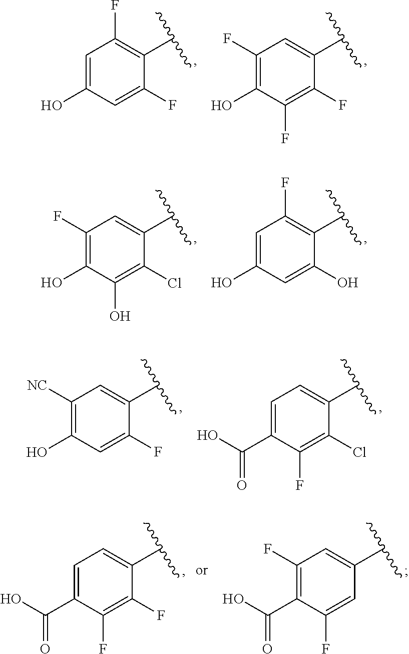

each Y¹ and Y² is independently —C(═O)— or —C(R⁵)₂—;
R⁴ is —S(═O)₂R^{b}, —S(═O)₂NR^{c}R^{d}, or —C(═O)R^{b}, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R⁵ is independently hydrogen, halogen, —OH, —CN, NH₂, NO₂, optionally substituted alkyl, optionally substituted alkoxy, optionally substituted cycloalkyl, optionally substituted heterocycloalkyl, optionally substituted aryl, or optionally substituted heteroaryl;
q is 1-3;
p is 1-3;
each R is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, optionally substituted heteroaryl, halogen, —CN, —OR^{a}, —SR^{a}, —NR^{c}R^{d}, —NR^{c}C(═O)R^{b}, —C(═O)NR^{c}R^{d}, —C(═O)R^{a}, or —C(═O)OR^{a};
m is 0-3;
R^{d} is hydrogen or C₁-C₆ alkyl;
each R^{e} is independently hydrogen, —CN, —OH, C₁-C₆ alkyl, or cycloalkyl;
X¹ and X² are independently —OH, —OR^{X}, or F; or
X¹ and X² are taken together with the boron atom to which there are attached to form an optionally substituted cyclic boronate ester;
R^{X} is C₁-C₆ alkyl or cycloalkyl;
Z is hydrogen, R⁶¹, (R⁶⁰)_{q}OR⁶¹, —(R⁶⁰)_{q}O(R⁶⁰)_{q}OR⁶¹, —R⁶⁰OC(═O)R⁶¹, —R⁶⁰OC(═O)OR⁶¹, —R⁶⁰OC(═O)NHR⁶¹, —R⁶⁰OC(═O)N(R⁶¹)₂, optionally substituted alkyloxyalkyl, optionally substituted acyloxyalkyl, optionally substituted alkyloxycarbonyloxyalkyl, optionally substituted cycloalkyloxycarbonyloxyalkyl, optionally substituted aryloxycarbonyloxyalkyl, or optionally substituted alkyl-[1,3]dioxol-2-one;
each R⁶⁰ is independently —CH₂—, —CH(CH₃)—, —C(CH₃)₂—, or 1,1′-cyclopropylene;
each R⁶¹ is independently optionally substituted C₁-C₆ alkyl, optionally substituted cycloalkyl, optionally substituted heterocycloalkyl, optionally substituted aryl, or optionally substituted heteroaryl; or
two R⁶¹ are taken together with the nitrogen to which they are attached to form an optionally substituted heterocycloalkyl;
each R^{a} is independently hydrogen, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{b} is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{c} and R^{d} are independently hydrogen, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl; or
each R^{c} and R^{d} are taken together with the nitrogen to which they are attached to form an optionally substituted heterocycloalkyl.

Provided herein are compounds of Formula (Ia) or (Ib), or a pharmaceutically acceptable salt, solvate, stereoisomer, tautomer, N-oxide, dimer, or trimer thereof:

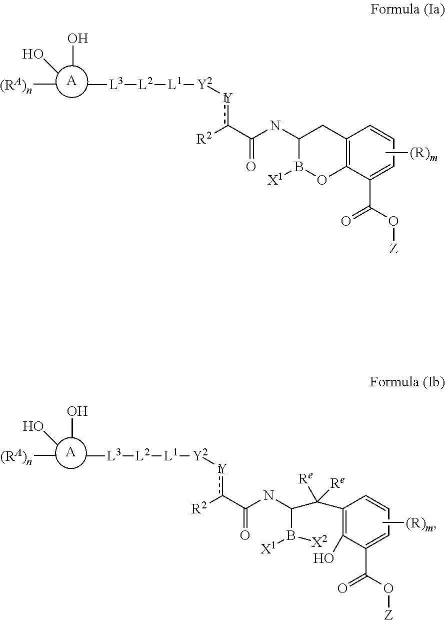

wherein:

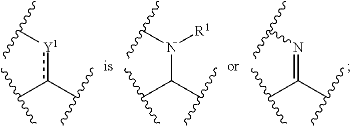

R¹ is hydrogen or C₁-C₆ alkyl;
Y² is —(C═O)— or —O—;
L¹ is absent, cycloalkyl, heterocycloalkyl, aryl, or heteroaryl; wherein the cycloalkyl, heterocycloalkyl, aryl, and heteroaryl is optionally substituted with one to six R^{L1};
each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or
two R^{L1} on the same carbon are taken together to form an oxo;
L² is absent or C₁-C₆ alkylene optionally substituted with one, two, or three R^{L2};
each R^{L2} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a};
L³ is absent, —C(═O)NH—, —NHC(═O)—, or —NH—;
Ring A is cycloalkyl, heterocycloalkyl, aryl, or heteroaryl;
each R^{A} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a};
n is 0-3;
R² is hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl is optionally substituted with one, two, or three R²;
each R^{2′} is independently C₁-C₆ alkyl, C₁-C₆ haloalkyl, C₁-C₆ hydroxyalkyl, C₁-C₆ aminoalkyl, halogen, —CN, —OR^{a}, —NR^{c}R^{d}, —NR^{c}C(═O)R^{b}, —C(═O)NR^{c}R^{d}, —C(═O)R^{b}, —C(═O)OR^{a}, cycloalkyl, heterocycloalkyl, aryl, or heteroaryl;
each R is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, optionally substituted heteroaryl, halogen, —CN, —OR^{a}, —SR^{a}, —NR^{c}R^{d}, —NR^{c}C(═O)R^{b}, —C(═O)NR^{c}R^{d}, —C(═O)R^{a}, or —C(═O)OR^{a};
m is 0-3;
R^{d} is hydrogen or C₁-C₆ alkyl;
each R^{e} is independently hydrogen, —CN, —OH, C₁-C₆ alkyl, or cycloalkyl;
X¹ and X² are independently —OH, —OR^{X}, or F; or
X¹ and X² are taken together with the boron atom to which there are attached to form an optionally substituted cyclic boronate ester;
R^{X} is C₁-C₆ alkyl or cycloalkyl;
Z is hydrogen, R⁶¹, (R⁶⁰)_{q}OR⁶¹, —(R⁶⁰)_{q}O(R⁶⁰)_{q}OR⁶¹, —R⁶⁰OC(═O)R⁶¹, —R⁶⁰OC(═O)OR⁶¹, —R⁶⁰OC(═O)NHR⁶¹, —R⁶⁰OC(═O)N(R⁶¹)₂, optionally substituted alkyloxyalkyl, optionally substituted acyloxyalkyl, optionally substituted alkyloxycarbonyloxyalkyl, optionally substituted cycloalkyloxycarbonyloxyalkyl, optionally substituted aryloxycarbonyloxyalkyl, or optionally substituted alkyl-[1,3]dioxol-2-one;
each R⁶⁰ is independently —CH₂—, —CH(CH₃)—, —C(CH₃)₂—, or 1,1′-cyclopropylene;
each R⁶¹ is independently optionally substituted C₁-C₆ alkyl, optionally substituted cycloalkyl, optionally substituted heterocycloalkyl, optionally substituted aryl, or optionally substituted heteroaryl; or two R⁶¹ are taken together with the nitrogen to which they are attached to form an optionally substituted heterocycloalkyl;
each R^{a} is independently hydrogen, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{b} is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{c} and R^{d} are independently hydrogen, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl; or
each R^{c} and R^{d} are taken together with the nitrogen to which they are attached to form an optionally substituted heterocycloalkyl.

Also provided herein are of Formula (Ia′) or (Ib′), or a pharmaceutically acceptable salt, solvate, stereoisomer, tautomer, N-oxide, dimer, or trimer thereof:

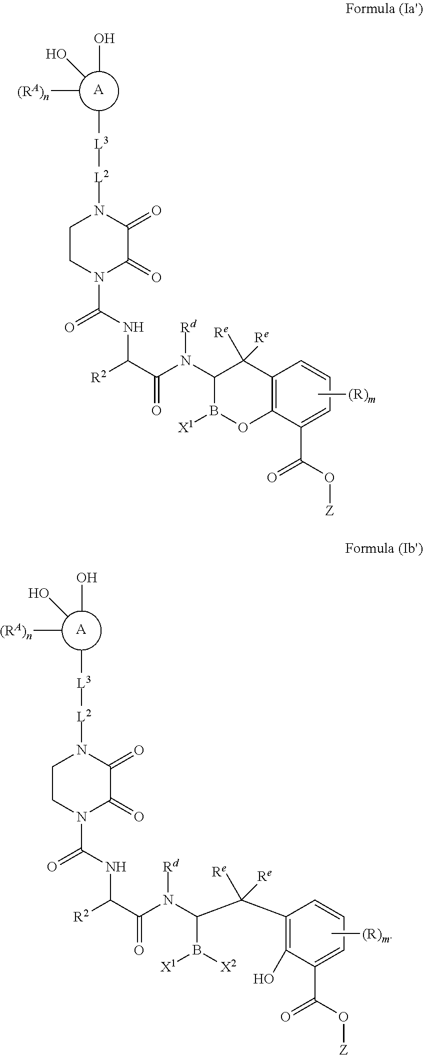

Also disclosed herein is a pharmaceutical composition comprising a compound disclosed herein and a pharmaceutically acceptable excipient.

Also disclosed herein is a method of treating a bacterial infection in a subject, comprising administering to the subject an effective amount of a compound disclosed herein, or a pharmaceutically acceptable salt, solvate, stereoisomer, tautomer, N-oxide, dimer, or trimer thereof, or a pharmaceutical composition comprising a compound disclosed herein and a pharmaceutically acceptable excipient.

Also disclosed herein is method of inhibiting a bacterial penicillin binding protein in a human infected with a bacterial infection, comprising contacting said bacterial penicillin binding protein with an effective amount of compound disclosed herein, or a pharmaceutically acceptable salt, solvate, stereoisomer, tautomer, N-oxide, dimer, or trimer thereof, or a pharmaceutical composition comprising a compound disclosed herein and a pharmaceutically acceptable excipient.

In some embodiments, the bacterial infection is caused by Neisseria gonorrhoeae. In some embodiments, the bacterial infection is caused by Pseudomonas aeruginosa. In some embodiments, the bacterial infection is caused by Acinetobacter baumannii. In some embodiments, the bacterial infection is caused by Pseudomonas aeruginosa Acinetobacter baumannii. In some embodiments, the bacterial infection is caused by a carbapenem-resistant enterobacteriaceae (CRE).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Over the decades of clinical use of beta-lactam antibiotics, bacteria have evolved resistance mechanisms that compromise beta-lactam utility, including production of easily transferable, broad-spectrum beta-lactamases that are able to efficiently hydrolyze the beta lactam ring. These enzymes, now counting >1300 variants, have spread throughout Enterobacteriaceae. The rapid spread of this mechanism of bacterial resistance severely limits beta-lactam therapeutic options. Novel non-beta-lactam compounds that inhibit the transpeptidase function of PBPs and are not degraded by beta-lactamases would represent a major advance in the treatment of resistant bacterial infections, essentially circumventing >70 years of bacterial evolution to protect the function of the penicillin-binding proteins in cell wall biosynthesis. The present invention is directed to certain boron-based compounds (boronic acids and cyclic boronic acid esters) which are PBP inhibitors and antibacterial compounds. The compounds and their pharmaceutically acceptable salts are useful for the treatment of bacterial infections, particularly antibiotic resistant bacterial infections. Some embodiments include compounds, compositions, pharmaceutical compositions, use, and preparation thereof.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The term “antibiotic” refers to a compound or composition which decreases the viability of a microorganism, or which inhibits the growth or proliferation of a microorganism. The phrase “inhibits the growth or proliferation” means increasing the generation time (i.e., the time required for the bacterial cell to divide or for the population to double) by at least about 2-fold. Preferred antibiotics are those which can increase the generation time by at least about 10-fold or more (e.g., at least about 100-fold or even indefinitely, as in total cell death). As used in this disclosure, an antibiotic is further intended to include an antimicrobial, bacteriostatic, or bactericidal agent. Examples of antibiotics suitable for use with respect to the present invention include penicillins, cephalosporins, and carbapenems.

The term “β-lactam antibiotic” refers to a compound with antibiotic properties that contains a β-lactam functionality. Non-limiting examples of β-lactam antibiotics useful with respect to the invention include penicillins, cephalosporins, penems, carbapenems, and monobactams.

The term “β-lactamase” denotes a protein capable of inactivating a β-lactam antibiotic. The β-lactamase can be an enzyme which catalyzes the hydrolysis of the β-lactam ring of a β-lactam antibiotic. Of particular interest herein are microbial β-lactamases. The β-lactamase may be, for example, a serine β-lactamase or a metallo-β-lactamase.

The term “penicillin-binding protein” (“PBP”) refers to a family of indispensable bacterial enzymes responsible for the synthesis of peptidoglycan, an essential structural polymer found exclusively in the cell wall of bacteria. This family of proteins encompasses three class. Class A are high molecular weight bifunctional enzymes possessing both glycosyltransferase (GTase) and transpeptidase (TPase) activities, while class B are monofunctional high molecular weight transpeptidases and class C are low molecular weight remodeling enzymes that include D,D-carboxypeptidases and D,D-endopeptidases. Penicillin binding proteins (PBPs) are the targets of β-lactam antibiotics, agents that covalently modify the active site of TPases and block the synthesis and remodeling of peptidoglycan, leading to rapid bacterial cell lysis of actively dividing cells.

“Amino” refers to the —NH₂ substituent.

“Oxo” refers to the ═O substituent.

“Oxime” refers to the ═N—OH substituent.

“Thioxo” refers to the ═S substituent.

“Alkyl” refers to a linear or branched hydrocarbon chain, which is fully saturated. Alkyl may have from one to thirty carbon atoms. An alkyl comprising up to 30 carbon atoms is referred to as a C₁-C₃₀ alkyl, likewise, for example, an alkyl comprising up to 12 carbon atoms is a C₁-C₁₂ alkyl. An alkyl comprising up to 6 carbons is a C₁-C₆ alkyl. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆alkyl, C₁-C₄ alkyl, C₁-C₃ alkyl, C₁-C₂ alkyl, C₂-C₈ alkyl, C₃-C₈ alkyl, C₄-C₈ alkyl, and C₅-C₁₂ alkyl. In some embodiments, the alkyl group is C₁-C₆ alkyl. Representative alkyl groups include, but are not limited to, methyl, ethyl, n-propyl, 1-methylethyl (isopropyl), n-butyl, i-butyl, s-butyl, n-pentyl, 1,1-dimethylethyl (t-butyl), 2-ethylpropyl, and the like. Representative linear alkyl groups include, but are not limited to, methyl, ethyl, n-propyl, n-butyl, n-pentyl and the like. In some embodiments, the alkyl is substituted with an optionally substituted aryl to form an optionally substituted aralkyl. In some embodiments, the alkyl is substituted with an optionally substituted heteroaryl to form an optionally substituted heteroarylalkyl. In some embodiments, the alkyl is substituted with an optionally substituted cycloalkyl to form an optionally substituted cycloalkylalkyl. In some embodiments, the alkyl is substituted with an optionally substituted heterocycloalkyl to form an optionally substituted heterocycloalkylalkyl. In some embodiments, the alkyl group is optionally substituted with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, -OMe, —NH₂, or —NO₂. In some embodiments, the alkyl is optionally substituted with oxo, halogen, —CN, —CF₃, —OH, or -OMe. In some embodiments, the alkyl is optionally substituted with halogen.

“Alkenyl” refers to a straight or branched hydrocarbon chain, containing at least one carbon-carbon double bond. In certain embodiments, alkenyl comprises two to twelve (C₂-C₁₂ alkenyl) carbon atoms, or two to eight carbon atoms (C₂-C₈ alkenyl), or two to six carbon atoms (C₂-C₆ alkenyl) or two to four carbon atoms (C₂-C₄ alkenyl). The alkenyl may be attached to the rest of the molecule by a single bond, for example, ethenyl (i.e., vinyl), prop-1-enyl (i.e., allyl), but-1-enyl, pent-1-enyl, penta-1,4-dienyl, and the like. Alkenyl may be attached to the rest of the molecule by a double bond, e.g., ═CH₂, ═CH(CH₂)₃CH₃. In some embodiments, the alkenyl group is optionally substituted with oxo, halogen, amino, nitrile, nitro, hydroxyl, haloalkyl, alkoxy, aryl, cycloalkyl, heterocycloalkyl, heteroaryl, and the like. In some embodiments, the alkenyl is optionally substituted with oxo, halogen, —CN, —CF₃, —OH, -OMe, —NH₂, or —NO₂. In some embodiments, the alkenyl is optionally substituted with oxo, halogen, —CN, —CF₃, —OH, or -OMe. In some embodiments, the alkenyl is optionally substituted with halogen.

“Alkynyl” refers to a straight or branched hydrocarbon chain group, containing at least one carbon-carbon triple bond. In certain embodiments, alkynyl comprises two to twelve (C₂-C₁₂ alkynyl) carbon atoms, or two to eight carbon atoms (C₂-C₈ alkynyl), or two to six carbon atoms (C₂-C₆ alkynyl) or two to four carbon atoms (C₂-C₄ alkynyl). The alkynyl may be attached to the rest of the molecule by a single bond, for example, ethynyl, propynyl, butynyl, pentynyl, hexynyl, and the like. In some embodiments, the alkynyl group is optionally substituted with oxo, halogen, amino, nitrile, nitro, hydroxyl, haloalkyl, alkoxy, aryl, cycloalkyl, heterocycloalkyl, heteroaryl, and the like. In some embodiments, the alkynyl is optionally substituted with oxo, halogen, —CN, —CF₃, —OH, -OMe, —NH₂, or —NO₂. In some embodiments, the alkynyl is optionally substituted with oxo, halogen, —CN, —CF₃, —OH, or -OMe. In some embodiments, the alkynyl is optionally substituted with halogen.

“Alkylene” or “alkylene chain” refers to a straight or branched divalent hydrocarbon chain linking the rest of the molecule to a radical group, consisting solely of carbon and hydrogen, containing no unsaturation and having, for example, from one to twelve carbon atoms (C₁-C₁₂ alkylene), e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (C₁-C₈ alkylene), or one to five carbon atoms (C₁-C₅ alkylene), or one to four carbon atoms (C₁-C₄ alkylene), or one to three carbon atoms (C₁-C₃ alkylene), or one to two carbon atoms (C₁-C₂ alkylene). In other embodiments, an alkylene comprises one carbon atom (C₁ alkylene), or two carbon atoms (C₂ alkylene). In certain embodiments, an alkylene comprises two to five carbon atoms (e.g., C₂-C₅ alkylene). In some embodiments, the alkylene is optionally substituted with oxo, halogen, —CN, —CF₃, —OH, -OMe, —NH₂, or —NO₂. In some embodiments, the alkylene is optionally substituted with oxo, halogen, —CN, —CF₃, —OH, or -OMe. In some embodiments, the alkylene is optionally substituted with halogen.

“Alkoxy” refers to a radical of the formula —O-alkyl where alkyl is as defined herein. Unless stated otherwise specifically in the specification, an alkoxy group may be optionally substituted as described above for alkyl.

“Aryl” refers to an aromatic monocyclic hydrocarbon or aromatic multicyclic hydrocarbon ring system by removing a hydrogen atom from a ring carbon atom. Aryl may include cycles with six to eighteen carbon atoms, where at least one of the rings in the ring system is aromatic, i.e., it contains a cyclic, delocalized (4n+2) π-electron system in accordance with the Hückel theory. In some embodiments, the aryl is a monocyclic, bicyclic, tricyclic or tetracyclic ring system, which may include fused ring system (when fused with a cycloalkyl or heterocycloalkyl ring, the aryl is bonded through an aromatic ring atom). In some embodiments, the aryl is a 6 to 10-membered aryl. In some embodiments, the aryl is a 6-membered aryl. In some embodiments, the aryl is a 10-membered aryl. The ring system from which aryl groups are derived include, but are not limited to, groups such as benzene, fluorene, indane, indene, tetralin and naphthalene. In some embodiments, the aryl is optionally substituted with halogen, amino, nitrile, nitro, hydroxyl, alkyl, haloalkyl, alkoxy, aryl, aralkyl, cycloalkyl, heterocycloalkyl, heteroaryl, and the like. In some embodiments, the aryl is optionally substituted with halogen, —CN, -Me, -Et, —CF₃, —OH, -OMe, —NH₂, —NO₂, or cyclopropyl. In some embodiments, the aryl is optionally substituted with oxo, halogen, —CN, -Me, -Et, —CF₃, —OH, -OMe, or cyclopropyl. In some embodiments, the aryl is optionally substituted with halogen.

“Aryloxy” refers to a radical bonded through an oxygen atom of the formula —O-aryl, where aryl is as described above.

“Aralkyl” refers to a radical of the formula —Rh-aryl where Rh is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Boronate ester” refers to —B(OR^{k})₂ wherein each R^{k} are independently hydrogen, optionally substituted alkyl, optionally substituted cycloalkyl, optionally substituted heterocycloalkyl, optionally substituted aryl, optionally substituted heteroaryl, (poly ethylene glycol) ethyl, or an optionally substituted saccharide provided that they are not both hydrogen. In some embodiments, each R^{k} is alkyl. In some embodiments, two R^{k} may be taken together with the atom to which they are attached to form an optionally substituted heterocycle or a cyclic boronate ester. In some embodiments, the cyclic boronate ester is formed from pinanediol, pinacol, 1,2-ethanediol, 1,3-propanediol, 1,2-propanediol, 2,3-butanediol, 1,2-diisopropylethandiol, 5,6-decanediol, 1,2-dicyclohexylethanediol, diethanolamine, 1,2-diphenyl-1,2-ethanediol, 2,6,6-trimethylbicyclo[3.1.1]heptane-2,3-diol, or (1S,2S,3R,5S)-2,6,6-trimethylbicyclo[3.1.1]heptane-2,3-diol.

“Cycloalkyl” refers to a saturated or partially unsaturated, monocyclic or polycyclic hydrocarbon. In certain embodiments, the cycloalkyl includes fused (when fused with an aryl or a heteroaryl ring, the cycloalkyl is bonded through a non-aromatic ring atom) or bridged ring systems. In certain embodiments, the cycloalkyl comprises from three to twenty carbon atoms (C₃-C₂₀ cycloalkyl), or three to ten carbon atoms (C₃-C₁₀ cycloalkyl), or three to eight carbon atoms (C₃-C₈ cycloalkyl), or three to six carbon atoms (C₃-C₆ cycloalkyl). In some embodiments, the cycloalkyl is a 3- to 6-membered cycloalkyl. In some embodiments, the cycloalkyl is a 3- to 8-membered cycloalkyl. Examples of monocyclic cycloalkyls include, e.g., cyclopropyl, cyclobutyl, cyclopentyl, cyclohexyl, cycloheptyl, and cyclooctyl. Polycyclic cycloalkyl include, for example, adamantyl, norbornyl (i.e., bicyclo[2.2.1]heptanyl), norbornenyl, decalinyl, 7,7-dimethyl-bicyclo[2.2.1]heptanyl, and the like. In some embodiments, the cycloalkyl is optionally substituted with oxo, halogen, amino, nitrile, nitro, hydroxyl, alkyl, haloalkyl, alkoxy, aryl, aralkyl, cycloalkyl, heterocycloalkyl, heteroaryl, and the like. In some embodiments, the cycloalkyl is optionally substituted with oxo, halogen, —CN, -Me, -Et, —CF₃, —OH, -OMe, —NH₂, —NO₂, or cyclopropyl. In some embodiments, the cycloalkyl is optionally substituted with oxo, halogen, —CN, -Me, -Et, —CF₃, —OH, -OMe, or cyclopropyl. In some embodiments, the cycloalkyl is optionally substituted with halogen.

“Cycloalkylalkyl” refers to a radical of the formula —Rh-cycloalkyl where Rh is an alkylene chain as defined above. The alkylene chain and the cycloalkyl radical are optionally substituted as described above.

“Halo” or “halogen” refers to bromo, chloro, fluoro, or iodo. In some embodiments, halogen refers to chloro or fluoro.

“Heterocycloalkyl” refers to a saturated or partially unsaturated ring that comprises two to twenty carbon atoms and at least one heteroatom. In certain embodiments, the heteroatoms are independently selected from N, O, Si, P, B, and S atoms. In certain embodiments, the heteroatoms are independently selected from N, O, and S atoms. The heterocycloalkyl may be selected from monocyclic or bicyclic, fused (when fused with an aryl or a heteroaryl ring, the heterocycloalkyl is bonded through a non-aromatic ring atom) or bridged ring systems. The heteroatoms in the heterocycloalkyl are optionally oxidized. One or more nitrogen atoms, if present, are optionally quaternized. The heterocycloalkyl is partially or fully saturated. The heterocycloalkyl is attached to the rest of the molecule through any atom of the heterocycloalkyl, valence permitting, such as any carbon or nitrogen atoms of the heterocycloalkyl. In certain embodiments, the heterocycloalkyl comprises from two to twenty carbon atoms (C₂-C₂₀ heterocycloalkyl), or two to ten carbon atoms (C₂-C₁₀ heterocycloalkyl), or two to eight carbon atoms (C₂-C₈ heterocycloalkyl), or two to six carbon atoms (C₂-C₆ heterocycloalkyl). In some embodiments, the heterocycloalkyl is a 3- to 6-membered heterocycloalkyl. In some embodiments, the heterocycloalkyl is a 3- to 8-membered heterocycloalkyl. In some embodiments, the heterocycloalkyl is a 5- to 6-membered heterocycloalkyl. In some embodiments, the heterocycloalkyl is a 5-membered heterocycloalkyl. In some embodiments, the heterocycloalkyl is a 6-membered heterocycloalkyl. Examples of heterocycloalkyl include, but are not limited to, azetidinyl, aziridyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocycloalkyl is optionally substituted with oxo, halogen, amino, nitrile, nitro, hydroxyl, alkyl, haloalkyl, alkoxy, aryl, aralkyl, cycloalkyl, heterocycloalkyl, heteroaryl, and the like. In some embodiments, the heterocycloalkyl is optionally substituted with oxo, halogen, —CN, -Me, -Et, —CF₃, —OH, -OMe, —NH₂, —NO₂, or cyclopropyl. In some embodiments, the heterocycloalkyl is optionally substituted with oxo, halogen, —CN, -Me, -Et, —CF₃, —OH, -OMe, or cyclopropyl. In some embodiments, the heterocycloalkyl is optionally substituted with halogen.

“Heterocycloalkylalkyl” refers to a radical of the formula —Rh-heterocycloalkyl where Rh is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylalkyl radical is optionally substituted as defined above for an alkylene chain. The heterocycloalkyl part of the heterocycloalkylalkyl radical is optionally substituted as defined above for a heterocycloalkyl group.

“Heteroaryl” refers to a 5- to 14-membered ring system comprising hydrogen atoms, one to thirteen carbon atoms, one to six heteroatoms selected from the group consisting of nitrogen, oxygen, phosphorous and sulfur, and at least one aromatic ring. In some embodiments, the heteroaryl is a 5- or 6-membered heteroaryl. In some embodiments, the heteroaryl is a 5-membered heteroaryl. In some embodiments, the heteroaryl is a 6-membered heteroaryl. In some embodiments, the heteroaryl is a monocyclic, bicyclic, tricyclic or tetracyclic ring system, which may include fused ring systems (when fused with a cycloalkyl or heterocycloalkyl ring, the heteroaryl is bonded through an aromatic ring atom); and the nitrogen, carbon or sulfur atoms in the heteroaryl may be optionally oxidized; the nitrogen atom may be optionally quaternized. In some embodiments, the heteroaryl is a 5- to 10-membered heteroaryl. In some embodiments, the heteroaryl is a 10-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). In some embodiments, the heteroaryl is optionally substituted with halogen, amino, nitrile, nitro, hydroxyl, alkyl, haloalkyl, alkoxy, aryl, aralkyl, cycloalkyl, heterocycloalkyl, heteroaryl, and the like. In some embodiments, the heteroaryl is optionally substituted with halogen, —CN, -Me, -Et, —CF₃, —OH, -OMe, —NH₂, —NO₂, or cyclopropyl. In some embodiments, the heteroaryl is optionally substituted with halogen, —CN, -Me, -Et, —CF₃, —OH, -OMe, or cyclopropyl. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In some embodiments, treatment also includes prophylactic treatment (e.g., administration of a composition described herein when an individual is suspected to be suffering from a bacterial infection).

### Compounds

Described herein are compounds that modulate the activity of penicillin-binding proteins. In some embodiments, the compounds described herein inhibit beta-lactamase. In certain embodiments, the compounds described herein are useful in the treatment of bacterial infections. In some embodiments, the bacterial infection is an upper or lower respiratory tract infection, a urinary tract infection, an intra-abdominal infection, or a skin infection. In some embodiments, the bacterial infection is uncomplicated or complicated urinary tract infections, uncomplicated or complicated gonorrhea, upper or lower respiratory tract infections, skin or skin structure infections, intra-abdominal infections, central nervous system infections, blood stream infections, or systemic infections.

Also disclosed herein is a compound of Formula (Ia) or (Ib), or a pharmaceutically acceptable salt, solvate, stereoisomer, tautomer, N-oxide, dimer, or trimer thereof:

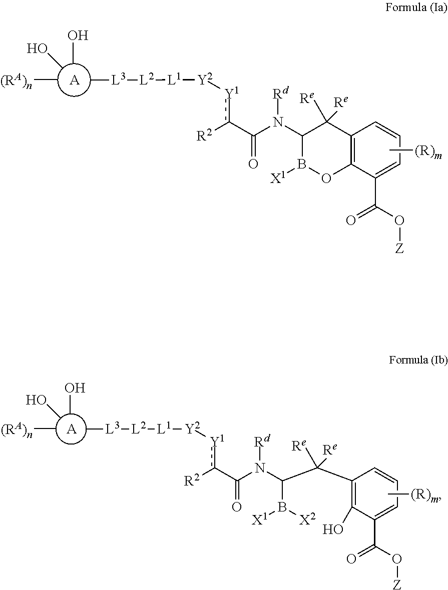

wherein:

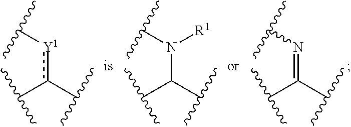

R¹ is hydrogen or C₁-C₆ alkyl;
Y² is —(C═O)— or —O—;
L¹ is absent, cycloalkyl, heterocycloalkyl, aryl, or heteroaryl; wherein the cycloalkyl, heterocycloalkyl, aryl, and heteroaryl is optionally substituted with one to six R^{L1};
each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or
two R^{L1} on the same carbon are taken together to form an oxo;
L² is absent or C₁-C₆ alkylene optionally substituted with one, two, or three R^{L2};
each R^{L2} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a};
L³ is absent, —C(═O)NH—, —NHC(═O)—, or —NH—;
Ring A is cycloalkyl, heterocycloalkyl, aryl, or heteroaryl;
each R^{A} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a};
n is 0-3;
R² is hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl is optionally substituted with one, two, or three R^{2′};
each R^{2′} is independently C₁-C₆ alkyl, C₁-C₆ haloalkyl, C₁-C₆ hydroxyalkyl, C₁-C₆ aminoalkyl, halogen, —CN, —OR^{a}, —NR^{c}R^{d}, —NR^{c}C(═O)R^{b}, —C(═O)NR^{c}R^{d}, —C(═O)R^{b}, —C(═O)OR^{a}, cycloalkyl, heterocycloalkyl, aryl, or heteroaryl;
each R is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, optionally substituted heteroaryl, halogen, —CN, —OR^{a}, —SR^{a}, —NR^{c}R^{d}, —NR^{c}C(═O)R^{b}, —C(═O)NR^{c}R^{d}, —C(═O)R^{a}, or —C(═O)OR^{a};
m is 0-3;
R^{d} is hydrogen or C₁-C₆ alkyl;
each R^{e} is independently hydrogen, —CN, —OH, C₁-C₆ alkyl, or cycloalkyl;
X¹ and X² are independently —OH, —OR^{X}, or F; or
X¹ and X² are taken together with the boron atom to which there are attached to form an optionally substituted cyclic boronate ester;
R^{X} is C₁-C₆ alkyl or cycloalkyl;
Z is hydrogen, R⁶¹, (R⁶⁰)_{q}OR⁶¹, —(R⁶⁰)_{q}O(R⁶⁰)_{q}OR⁶¹, —R⁶⁰OC(═O)R⁶¹, —R⁶⁰OC(═O)OR⁶¹, —R⁶⁰OC(═O)NHR⁶¹, —R⁶⁰OC(═O)N(R⁶¹)₂, optionally substituted alkyloxyalkyl, optionally substituted acyloxyalkyl, optionally substituted alkyloxycarbonyloxyalkyl, optionally substituted cycloalkyloxycarbonyloxyalkyl, optionally substituted aryloxycarbonyloxyalkyl, or optionally substituted alkyl-[1,3]dioxol-2-one;
each R⁶⁰ is independently —CH₂—, —CH(CH₃)—, —C(CH₃)₂—, or 1,1′-cyclopropylene;
each R⁶¹ is independently optionally substituted C₁-C₆ alkyl, optionally substituted cycloalkyl, optionally substituted heterocycloalkyl, optionally substituted aryl, or optionally substituted heteroaryl; or
two R⁶¹ are taken together with the nitrogen to which they are attached to form an optionally substituted heterocycloalkyl;
each R^{a} is independently hydrogen, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{b} is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{c} and R^{d} are independently hydrogen, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl; or
each R^{c} and R^{d} are taken together with the nitrogen to which they are attached to form an optionally substituted heterocycloalkyl.

In some embodiments of a compound of Formula (Ia) or (Ib),

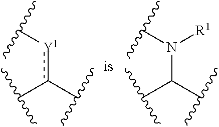

### and Y² is —(C═O)—.

In some embodiments of a compound of Formula (Ia) or (Ib), R¹ is hydrogen. In some embodiments of a compound of Formula (Ia) or (Ib), R¹ is C₁-C₆ alkyl.

In some embodiments of a compound of Formula (Ia) or (Ib),

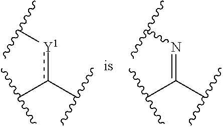

### and Y² is —O—.

In some embodiments of a compound of Formula (Ia) or (Ib), L¹ is absent.

In some embodiments of a compound of Formula (Ia) or (Ib), L¹ is heterocycloalkyl optionally substituted with one to six R^{L1}; and each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or two R^{L1} on the same carbon are taken together to form an oxo.

In some embodiments of a compound of Formula (Ia) or (Ib), L¹ is pyrrolidine, piperidine, or piperazine; each optionally substituted with one to six R^{L1}; and each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or two R^{L1} on the same carbon are taken together to form an oxo.

In some embodiments of a compound of Formula (Ia) or (Ib), L¹ is piperazine optionally substituted with one to six R^{L1}; and each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or two R^{L1} on the same carbon are taken together to form an oxo.

In some embodiments of a compound of Formula (Ia) or (Ib), L¹ is

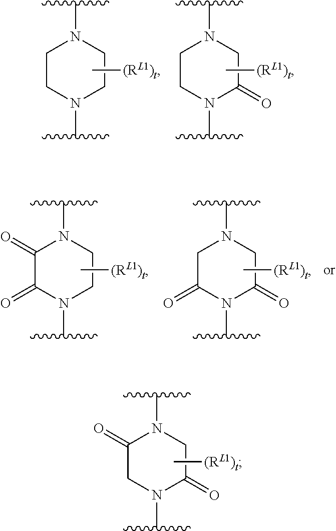

wherein each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}, and t is 0-2.

In some embodiments of a compound of Formula (Ia) or (Ib), L¹ is

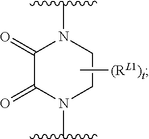

wherein each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; and t is 0-2.

In some embodiments of a compound of Formula (Ia) or (Ib), t is 0 or 1. In some embodiments of a compound of Formula (Ia) or (Ib), t is 1. In some embodiments of a compound of Formula (Ia) or (Ib), t is 0. In some embodiments of a compound of Formula (Ia) or (Ib), t is 2.

In some embodiments of a compound of Formula (Ia) or (Ib), each R^{L1} is independently halogen, C₁-C₆ alkyl, —CN, —OR^{a}, or —NR^{c}R^{d}; or two R^{L1} on the same carbon are taken together to form an oxo. In some embodiments of a compound of Formula (Ia) or (Ib), each R^{L1} is independently C₁-C₆ alkyl; or two R^{L1} on the same carbon are taken together to form an oxo. In some embodiments of a compound of Formula (Ia) or (Ib), each R^{L1} is independently C₁-C₆ alkyl. In some embodiments of a compound of Formula (Ia) or (Ib), two R^{L1} on the same carbon are taken together to form an oxo. In some embodiments of a compound of Formula (Ia) or (Ib), each R^{L1} is independently C₁-C₆ alkyl or two R^{L1} on the same carbon are taken together to form an oxo.

In some embodiments of a compound of Formula (Ia) or (Ib), L² is absent.

In some embodiments of a compound of Formula (Ia) or (Ib), L² is C₁-C₆ alkylene optionally substituted with one, two, or three R^{L2}; and each R^{L2} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}.

In some embodiments of a compound of Formula (Ia) or (Ib), each R^{L2} is independently halogen, C₁-C₆ alkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}. In some embodiments of a compound of Formula (Ia) or (Ib), each R^{L2} is independently halogen, C₁-C₆ alkyl, or —C(═O)OR^{a}.

In some embodiments of a compound of Formula (Ia) or (Ib), L² is C₂-C₄ alkylene. In some embodiments of a compound of Formula (Ia) or (Ib), L² is C₂-C₃ alkylene.

In some embodiments of a compound of Formula (Ia) or (Ib), L³ is absent.

In some embodiments of a compound of Formula (Ia) or (Ib), L³ is —C(═O)NH— or —NHC(═O)—.

In some embodiments of a compound of Formula (Ia) or (Ib), L³ is —NH—.

In some embodiments of a compound of Formula (Ia) or (Ib):
(a)^{L} is heterocycloalkyl optionally substituted with one to six R^{L1}; each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or two R^{L1} on the same carbon are taken together to form an oxo; L² is C₂-C₄ alkylene; and L³ is —C(═O)NH— or —NHC(═O)—; or
(b) L¹ is absent; L² is C₁-C₆ alkylene optionally substituted with one, two, or three R^{L2}; each R^{L2} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; and L³ is —C(═O)NH— or —NHC(═O)—; or
(c) L¹, L², and L³ are absent; or
(d) L¹ and L² are absent and L³ is —NH—.

In some embodiments of a compound of Formula (Ia) or (Ib), L¹ is heterocycloalkyl optionally substituted with one to six R^{L1}; each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or two R^{L1} on the same carbon are taken together to form an oxo; L² is C₂-C₄ alkylene; and L³ is —C(═O)NH— or —NHC(═O)—.

In some embodiments of a compound of Formula (Ia) or (Ib), L¹ is absent; L² is C₁-C₆ alkylene optionally substituted with one, two, or three R^{L2}; each R^{L2} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; and L³ is —C(═O)NH— or —NHC(═O)—; or

In some embodiments of a compound of Formula (Ia) or (Ib), L¹, L², and L³ are absent; or

In some embodiments of a compound of Formula (Ia) or (Ib), L¹ and L² are absent and L³ is —NH—.

In some embodiments of a compound of Formula (Ia) or (Ib), or a pharmaceutically acceptable salt, solvate, stereoisomer, tautomer, N-oxide, dimer, or trimer thereof; is of Formula (Ia′) or (Ib′):

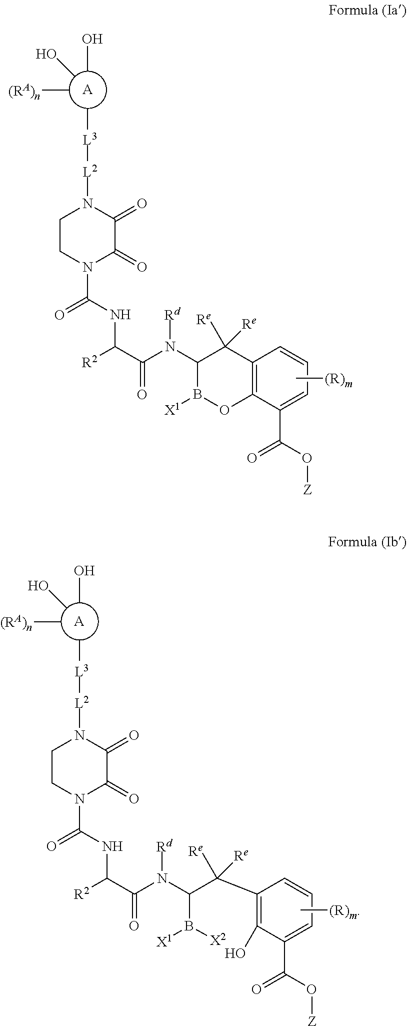

In some embodiments of a compound of Formula (Ia′) or (Ib′), L² is C₁-C₆ alkylene optionally substituted with one, two, or three R^{L2}; and each R^{L2} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}.

In some embodiments of a compound of Formula (Ia′) or (Ib′), L² is C₂-C₄ alkylene. In some embodiments of a compound of Formula (Ia′) or (Ib′), L² is C₂-C₃ alkylene.

In some embodiments of a compound of Formula (Ia′) or (Ib′), L³ is —C(═O)NH— or —NHC(═O)—.

In some embodiments of a compound of Formula (Ia), (Ib), (Ia′), or (Ib′), R^{d} is hydrogen.

In some embodiments of a compound of Formula (Ia), (Ib), (Ia′), or (Ib′), each R^{e} is hydrogen.

In some embodiments of a compound of Formula (Ia), (Ib), (Ia′), or (Ib′), X¹ and X² are —OH.

In some embodiments of a compound of Formula (Ia), (Ib), (Ia′), or (Ib′), each R is independently optionally substituted C₁-C₆ alkyl, optionally substituted C₁-C₆ haloalkyl, optionally substituted C₁-C₆ hydroxyalkyl, halogen, —CN, —OR^{a}, —SR^{a}, —NR^{c}R^{d}, —NR^{c}C(═O)R^{b}, —C(═O)NR^{c}R^{d}, —C(═O)R^{a}, or —C(═O)OR^{a}. In some embodiments of a compound of Formula (Ia), (Ib), (Ia′), or (Ib′), each R is independently optionally substituted C₁-C₆ alkyl, optionally substituted C₁-C₆ haloalkyl, halogen, —OR^{a}, —SR^{a}, or —C(═O)R^{a}. In some embodiments of a compound of Formula (Ia), (Ib), (Ia′), or (Ib′), each R is independently halogen.

In some embodiments of a compound of Formula (Ia), (Ib), (Ia′), or (Ib′), m is 0 or 1. In some embodiments of a compound of Formula (Ia), (Ib), (Ia′), or (Ib′), m is 1 or 2. In some embodiments of a compound of Formula (Ia), (Ib), (Ia′), or (Ib′), m is 1. In some embodiments of a compound of Formula (Ia), (Ib), (Ia′), or (Ib′), m is 2. In some embodiments of a compound of Formula (Ia), (Ib), (Ia′), or (Ib′), m is 0.

In some embodiments of a compound of Formula (Ia), (Ib), (Ia′), or (Ib′), Ring A is heteroaryl or aryl. In some embodiments of a compound of Formula (Ia), (Ib), (Ia′), or (Ib′), Ring A is heteroaryl. In some embodiments of a compound of Formula (Ia), (Ib), (Ia′), or (Ib′), Ring A is aryl.

In some embodiments of a compound of Formula (Ia), (Ib), (Ia′), or (Ib′), Ring A is phenyl.

In some embodiments of a compound of Formula (Ia), (Ib), (Ia′), or (Ib′),

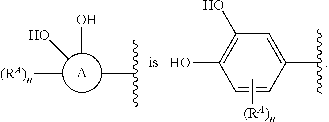

In some embodiments of a compound of Formula (Ia), (Ib), (Ia′), or (Ib′), each R^{A} is independently halogen or C₁-C₆ alkyl. In some embodiments of a compound of Formula (Ia), (Ib), (Ia′), or (Ib′), each R^{A} is independently halogen.

In some embodiments of a compound of Formula (Ia), (Ib), (Ia′), or (Ib′), n is 1 or 2. In some embodiments of a compound of Formula (Ia), (Ib), (Ia′), or (Ib′), n is 1. In some embodiments of a compound of Formula (Ia), (Ib), (Ia′), or (Ib′), n is 2. In some embodiments of a compound of Formula (Ia), (Ib), (Ia′), or (Ib′), n is 0.

In some embodiments of a compound of Formula (Ia), (Ib), (Ia′), or (Ib′), R² is C₁-C₆ alkyl, C₁-C₆ hydroxyalkyl, aryl, or heteroaryl; wherein the alkyl, aryl, and heteroaryl is optionally substituted with one, two, or three R^{2′}.

In some embodiments of a compound of Formula (Ia), (Ib), (Ia′), or (Ib′), R² is aryl or heteroaryl; wherein the aryl and heteroaryl is optionally substituted with one, two, or three R^{2′}.

In some embodiments of a compound of Formula (Ia), (Ib), (Ia′), or (Ib′), R² is aryl optionally substituted with one, two, or three R^{2′}. In some embodiments of a compound of Formula (Ia), (Ib), (Ia′), or (Ib′), R² is heteroaryl optionally substituted with one, two, or three R^{2′}.

In some embodiments of a compound of Formula (Ia), (Ib), (Ia′), or (Ib′), each R^{2′} is independently C₁-C₆ alkyl, halogen, —CN, —OR^{a}, —NR^{c}R^{d}, or —C(═O)OR^{a}. In some embodiments of a compound of Formula (Ia), (Ib), (Ia′), or (Ib′), each R^{2′} is independently halogen, —OR^{a}, —NR^{c}R^{d}, or —C(═O)OR^{a}.

In some embodiments of a compound of Formula (Ia), (Ib), (Ia′), or (Ib′), Z is hydrogen. In some embodiments of a compound of Formula (Ia), (Ib), (Ia′), or (Ib′), Z is R⁶⁰; and R⁶¹ is optionally substituted alkyl. In some embodiments of a compound of Formula (Ia), (Ib), (Ia′), or (Ib′), Z is —R⁶⁰OC(═O)R⁶¹ or —R⁶⁰OC(═O)OR⁶¹; R⁶⁰ is —CH₂— or —CH(CH₃)—; and R⁶¹ is optionally substituted alkyl, optionally substituted cycloalkyl, or optionally substituted heterocycloalkyl.

Also disclosed herein is a compound of Formula (IIa) or (IIb), or a pharmaceutically acceptable salt, solvate, stereoisomer, tautomer, N-oxide, dimer, or trimer thereof:

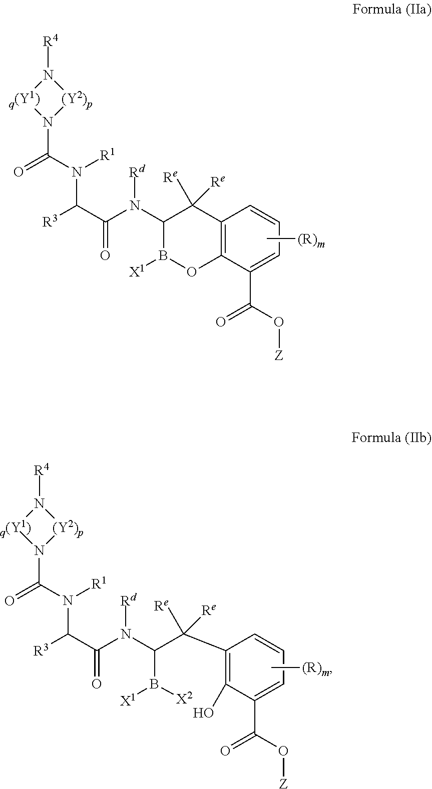

wherein:
R¹ is hydrogen or C₁-C₆ alkyl;
R³ is

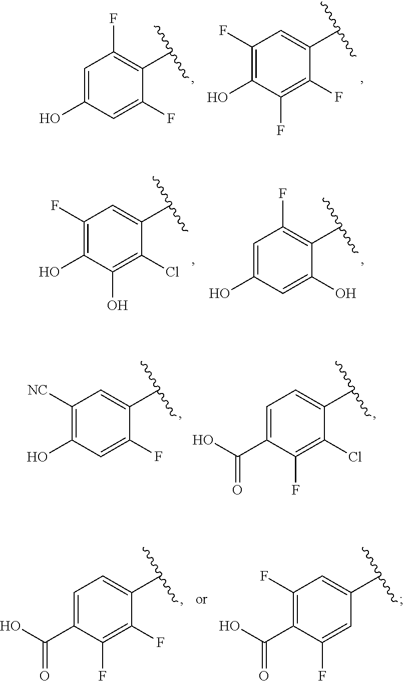

each Y¹ and Y² is independently —C(═O)— or —C(R⁵)₂—;
R⁴ is —S(═O)₂R^{b}, —S(═O)₂NR^{c}R^{d}, or —C(═O)R^{b}, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R⁵ is independently hydrogen, halogen, —OH, —CN, NH₂, NO₂, optionally substituted alkyl, optionally substituted alkoxy, optionally substituted cycloalkyl, optionally substituted heterocycloalkyl, optionally substituted aryl, or optionally substituted heteroaryl;
q is 1-3;
p is 1-3;
each R is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, optionally substituted heteroaryl, halogen, —CN, —OR^{a}, —SR^{a}, —NR^{c}R^{d}, —NR^{c}C(═O)R^{b}, —C(═O)NR^{c}R^{d}, —C(═O)R^{a}, or —C(═O)OR^{a};
m is 0-3;
R^{d} is hydrogen or C₁-C₆ alkyl;
each R^{e} is independently hydrogen, —CN, —OH, C₁-C₆ alkyl, or cycloalkyl;
X¹ and X² are independently —OH, —OR^{X}, or F; or
X¹ and X² are taken together with the boron atom to which there are attached to form an optionally substituted cyclic boronate ester;
R^{X} is C₁-C₆ alkyl or cycloalkyl;
Z is hydrogen, R⁶¹, (R⁶⁰)_{q}OR⁶¹, —(R⁶⁰)_{q}O(R⁶⁰)^{q}OR⁶¹, —R⁶⁰OC(═O)R⁶¹, —R⁶⁰OC(═O)OR⁶¹, —R⁶⁰OC(═O)NHR⁶¹, —R⁶⁰OC(═O)N(R⁶¹)₂, optionally substituted alkyloxyalkyl, optionally substituted acyloxyalkyl, optionally substituted alkyloxycarbonyloxyalkyl, optionally substituted cycloalkyloxycarbonyloxyalkyl, optionally substituted aryloxycarbonyloxyalkyl, or optionally substituted alkyl-[1,3]dioxol-2-one;
each R⁶⁰ is independently —CH₂—, —CH(CH₃)—, —C(CH₃)₂—, or 1,1′-cyclopropylene;
each R⁶¹ is independently optionally substituted C₁-C₆ alkyl, optionally substituted cycloalkyl, optionally substituted heterocycloalkyl, optionally substituted aryl, or optionally substituted heteroaryl; or
two R⁶¹ are taken together with the nitrogen to which they are attached to form an optionally substituted heterocycloalkyl;
each R^{a} is independently hydrogen, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{b} is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{c} and R^{d} are independently hydrogen, optionally substituted C₁-C₆ alkyl, optionally substituted C₁-C₆ haloalkyl, optionally substituted C₁-C₆ hydroxyalkyl, optionally substituted C1-C₆ aminoalkyl, optionally substituted cycloalkyl, optionally substituted heterocycloalkyl, optionally substituted aryl, or optionally substituted heteroaryl; or
each R^{c} and R^{d} are taken together with the nitrogen to which they are attached to form an optionally substituted heterocycloalkyl.

In some embodiments of a compound of Formula (IIa) or (IIb), R¹ is hydrogen. In some embodiments of a compound of Formula (IIa) or (IIb), R¹ is C₁-C₆ alkyl.

In some embodiments of a compound of Formula (IIa) or (IIb), R³ is

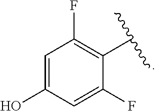 [SMILES: *c1c(F)cc(O)cc1F]

In some embodiments of a compound of Formula (IIa) or (IIb), R³ is

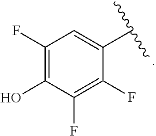 [SMILES: *c1cc(F)c(O)c(F)c1F]

In some embodiments of a compound of Formula (IIa) or (IIb), R³ is

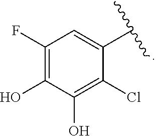 [SMILES: *c1cc(F)c(O)c(O)c1Cl]

In some embodiments of a compound of Formula (IIa) or (IIb), R³ is

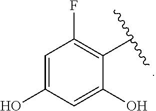 [SMILES: *c1c(O)cc(O)cc1F]

In some embodiments of a compound of Formula (IIa) or (IIb), R³ is

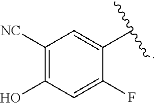 [SMILES: *c1cc(C#N)c(O)cc1F]

In some embodiments of a compound of Formula (IIa) or (IIb), R³ is

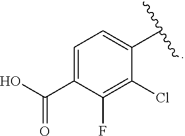 [SMILES: *c1ccc(C(=O)O)c(F)c1Cl]

In some embodiments of a compound of Formula (IIa) or (IIb), R³ is

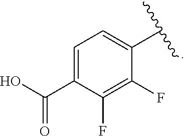 [SMILES: *c1ccc(C(=O)O)c(F)c1F]

In some embodiments of a compound of Formula (IIa) or (IIb), R³ is

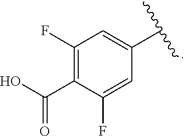 [SMILES: *c1cc(F)c(C(=O)O)c(F)c1]

In some embodiments of a compound of Formula (IIa) or (IIb), q is 2 and p is 2. In some embodiments of a compound of Formula (IIa) or (IIb), q is 2 and p is 1. In some embodiments of a compound of Formula (IIa) or (IIb), q is 1 and p is 1. In some embodiments of a compound of Formula (IIa) or (IIb), q is 1 and p is 2. In some embodiments of a compound of Formula (IIa) or (IIb), q is 3 and p is 2. In some embodiments of a compound of Formula (IIa) or (IIb), q is 3 and p is 1. In some embodiments of a compound of Formula (IIa) or (IIb), q is 3 and p is 3.

In some embodiments of a compound of Formula (IIa) or (IIb), q is 2; each Y¹ is —C(R⁵)₂—; p is 2; and each Y² is —C(═O)—. In some embodiments of a compound of Formula (IIa) or (IIb), q is 2; each Y¹ is —C(R⁵)₂—; p is 1; and Y² is —C(═O)—. In some embodiments of a compound of Formula (IIa) or (IIb), q is 2; each Y¹ is —C(R⁵)₂—; p is 2; and one Y² is —C(R⁵)₂— and one Y² is —C(═O)—. In some embodiments of a compound of Formula (IIa) or (IIb), q is 2; one Y¹ is —C(R⁵)₂— and one Y¹ is —C(═O)—; p is 2; and one Y² is —C(R⁵)₂— and one Y² is —C(═O)—.

In some embodiments of a compound of Formula (IIa) or (IIb), each R⁵ is independently hydrogen, halogen, or optionally substituted alkyl. In some embodiments of a compound of Formula (IIa) or (IIb), each R⁵ is hydrogen.

In some embodiments of a compound of Formula (IIa) or (IIb),

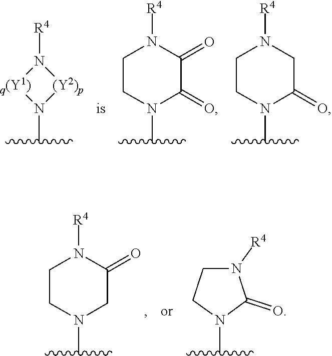

In some embodiments of a compound of Formula (IIa) or (IIb),

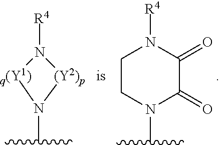

In some embodiments of a compound of Formula (IIa) or (IIb),

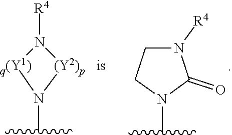

In some embodiments of a compound of Formula (IIa) or (IIb), R⁴ is —S(═O)₂R^{b}, optionally substituted C₁-C₆ alkyl, optionally substituted C₁-C₆ haloalkyl, optionally substituted C₁-C₆ hydroxyalkyl, or optionally substituted C₁-C₆ aminoalkyl. In some embodiments of a compound of Formula (IIa) or (IIb), R⁴ is optionally substituted C₁-C₆ alkyl or optionally substituted C₁-C₆ aminoalkyl. In some embodiments of a compound of Formula (IIa) or (IIb), R⁴ is C₁-C₆ alkyl or C₁-C₆ aminoalkyl. In some embodiments of a compound of Formula (IIa) or (IIb), R⁴ is C₁-C₆ alkyl. In some embodiments of a compound of Formula (IIa) or (IIb), R⁴ is C₁-C₆ aminoalkyl. In some embodiments of a compound of Formula (IIa) or (IIb), R⁴ is —S(═O)₂R^{b}.

In some embodiments of a compound of Formula (IIa) or (IIb), R^{d} is hydrogen.

In some embodiments of a compound of Formula (IIa) or (IIb), each R^{e} is hydrogen.

In some embodiments of a compound of Formula (IIa) or (IIb), X¹ and X² are —OH.

In some embodiments of a compound of Formula (IIa) or (IIb), each R is independently optionally substituted C₁-C₆ alkyl, optionally substituted C₁-C₆ haloalkyl, optionally substituted C₁-C₆ hydroxyalkyl, halogen, —CN, —OR^{a}, —SR^{a}, —NR^{c}R^{d}, —NR^{c}C(═O)R^{b}, —C(═O)NR^{c}R^{d}, —C(═O)R^{a}, or —C(═O)OR^{a}. In some embodiments of a compound of Formula (IIa) or (IIb), each R is independently optionally substituted C₁-C₆ alkyl, optionally substituted C₁-C₆ haloalkyl, halogen, —OR^{a}, —SR^{a}, or —C(═O)R^{a}. In some embodiments of a compound of Formula (IIa) or (IIb), each R is independently halogen.

In some embodiments of a compound of Formula (IIa) or (IIb), m is 0 or 1. In some embodiments of a compound of Formula (IIa) or (IIb), m is 1 or 2. In some embodiments of a compound of Formula (IIa) or (IIb), m is 1. In some embodiments of a compound of Formula (IIa) or (IIb), m is 2. In some embodiments of a compound of Formula (IIa) or (IIb), m is 0.

In some embodiments of a compound of Formula (IIa) or (IIb), Z is hydrogen. In some embodiments of a compound of Formula (IIa) or (IIb), Z is R⁶¹; and R⁶¹ is optionally substituted alkyl. In some embodiments of a compound of Formula (IIa) or (IIb), Z is —R⁶⁰OC(═O)R⁶¹ or —R⁶⁰OC(═O)OR⁶¹; R⁶⁰ is —CH₂— or —CH(CH₃)—; and R⁶¹ is optionally substituted alkyl, optionally substituted cycloalkyl, or optionally substituted heterocycloalkyl.

### Preparation of Compounds

Described herein are compounds that inhibit the activity of penicillin-binding proteins, and processes for their preparation. Also described herein are pharmaceutically acceptable salt, solvate, stereoisomer, tautomer, N-oxide, dimer, or trimer of compounds disclosed herein.

Compounds disclosed herein may be synthesized using standard synthetic reactions known to those of skill in the art or using methods known in the art. The reactions can be employed in a linear sequence to provide the compounds or they may be used to synthesize fragments which are subsequently joined by the methods known in the art.

The starting material used for the synthesis of the compounds described herein may be synthesized or can be obtained from commercial sources. The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989) (all of which are incorporated by reference in their entirety). General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein.

The products of the reactions may be isolated and purified, if desired, using conventional techniques, including, but not limited to, filtration, distillation, crystallization, chromatography, and the like. Such materials may be characterized using conventional means, including physical constants and spectral data.

### Further Forms of Compounds Disclosed Herein

### Isomers/Stereoisomers

In some embodiments, due to the oxophilic nature of the boron atom, the compounds described herein may convert to, or exist in equilibrium with, alternate forms, particularly in milieu that contain water (aqueous solution, plasma, etc.). Accordingly, the compounds described herein may exist in an equilibrium between the “closed” cyclic form shown in Formula (Ia), (Ia′), (IIa) and the “open” acyclic form shown in Formula (Ib), (Ib′), (IIb). In addition the compounds described herein may associate into intramolecular dimers, trimers, and related combinations.

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization. Compounds described herein may be prepared as a single isomer or a mixture of isomers.

### Tautomers

In some situations, compounds described herein exist as tautomers. The compounds described herein include all possible tautomers within the formulas described herein. A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid. In some embodiments, other acids, such as oxalic, while not in themselves pharmaceutically acceptable, are employed in the preparation of salts useful as intermediates in obtaining the compounds described herein and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran, or methanol. In addition, the compounds provided herein can exist in unsolvated as well as solvated forms. In general, the solvated forms are considered equivalent to the unsolvated forms for the purposes of the compounds and methods provided herein.

### Pharmaceutical Compositions/Formulations

In another aspect, provided herein are pharmaceutical compositions comprising a compound described herein, or a pharmaceutically acceptable salt, solvate, stereoisomer, tautomer, N-oxide, dimer, or trimer thereof, and a pharmaceutically acceptable excipient. In some embodiments, the pharmaceutical composition further comprises a beta-lactam antibiotic. In certain embodiments, the beta-lactam antibiotic is a penicillin, cephalosporin, carbapenem, monobactam, bridged monobactam, or a combination thereof.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound described herein with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination Treatment

The compounds described herein may be used in combination with one or more antibiotics in the treatment of bacterial infections. Such antibiotics may be administered, by a route and in an amount commonly used therefore, contemporaneously or sequentially with a compound described herein. When a compound described herein is used contemporaneously with one or more antibiotic, a pharmaceutical composition in unit dosage form containing such other drugs and the compound of the present invention is preferred. However, the combination therapy may also include therapies in which the compound described herein and one or more antibiotic are administered on different overlapping schedules. It is also contemplated that when used in combination with one or more antibiotics, the antibiotics may be used in lower doses than when each is used singly.

Accordingly, the pharmaceutical compositions of the present invention also include those that contain one or more antibiotics, in addition to a compound described herein. In some embodiments, a pharmaceutical composition comprising a compound described herein further comprises a beta-lactam antibiotic. In certain embodiments, the beta-lactam antibiotic is a penicillin, cephalosporin, carbapenem, monobactam, bridged monobactam, or a combination thereof.

In some embodiments, the compounds described herein are used in combination with one or more antibiotics in the treatment of bacterial infections. In certain embodiments, the bacterial infection is a upper or lower respiratory tract infection, a urinary tract infection, an intra-abdominal infection, or a skin infection. In some embodiments, the bacterial infection is an upper or lower respiratory tract infection, a urinary tract infection, an intra-abdominal infection, or a skin infection. In some embodiments, the bacterial infection is uncomplicated or complicated urinary tract infections, uncomplicated or complicated gonorrhea, upper or lower respiratory tract infections, skin or skin structure infections, intra-abdominal infections, central nervous system infections, blood stream infections, or systemic infections.

In some embodiments, the one or more antibiotics are selected from β-lactam antibiotics. β-Lactam antibiotics include, but are not limited to, penicillins, penems, carbapenems, cephalosporins, cephamycins, monobactams, or combinations thereof. Penicillins include, but are not limited to, amoxicillin, ampicillin, azidocillin, azlocillin, bacampicillin, benzathinebenzylpenicillin, benzathinephenoxymethylpenicillin, benzylpenicillin (G), carbenicillin, carindacillin, clometocillin, cloxacillin, dicloxacillin, epicillin, flucloxacillin, hetacillin, mecillinam, metampicillin, meticillin, mezlocillin, nafcillin, oxacillin, penamecillin, pheneticillin, phenoxymethylpenicillin (V), piperacillin, pivampicillin, pivmecillinam, procaine benzylpenicillin, propicillin, sulbenicillin, talampicillin, temocillin, and ticarcillin. Penems include, but are not limited to, faropenem. Carbapenems include, but are not limited to, biapenem, ertapenem, doripenem, imipenem, meropenem, and panipenem. Cephalosporins/Cephamycins include, but are not limited to, cefacetrile, cefaclor, cefadroxil, cefalexin, cefaloglycin, cefalonium, cefaloridine, cefalotin, cefamandole, cefapirin, cefatrizine, cefazaflur, cefazedone, cefazolin, cefbuperazone, cefcapene, cefdaloxime, cefdinir, cefditoren, cefepime, cefetamet, cefixime, cefmenoxime, cefmetazole, cefminox, cefodizime, cefonicid, cefoperazone, ceforanide, cefotaxime, cefotetan, cefotiam, cefovecin, cefoxitin, cefozopran, cefpimizole, cefpiramide, cefpirome, cefpodoxime, cefprozil, cefquinome, cefquinome, cefradine, cefroxadine, cefsulodin, ceftarolinefosamil, ceftazidime, cefteram, ceftezole, ceftibuten, ceftiofur, ceftiolene, ceftizoxime, ceftobiprole, ceftriaxone, cefuroxime, cefuzonam, flomoxef, latamoxef, and loracarbef. Monobactams include, but are not limited to, aztreonam, carumonam, nocardicinA, and tigemonam.

### Methods

The present disclosure also provides methods for inhibiting bacterial growth, such methods comprising contacting a bacterial cell culture, or a bacterially infected cell culture, tissue, or organism, with a penicillin-binding protein inhibitor described herein. Preferably, the bacteria to be inhibited by administration of a penicillin-binding protein inhibitor described herein are bacteria that are resistant to beta-lactam antibiotics. The term “resistant” is well-understood by those of ordinary skill in the art (see, e g Payne et al., Antimicrobial Agents and Chemotherapy 38 767-772 (1994), Hanaki et al., Antimicrobial Agents and Chemotherapy 30 1120-1126 (1995)). In some embodiments, the penicillin-binding protein inhibitor described herein is used to treat a bacterial infection that is resistant to beta-lactam antibiotic. In some embodiments, the penicillin-binding protein inhibitor described herein is used to treat a bacterial infection that has developed beta-lactamase enzymes.

These methods are useful for inhibiting bacterial growth in a variety of contexts. In certain embodiments, a compound described herein is administered to an experimental cell culture in vitro to prevent the growth of beta-lactam resistant bacteria. In some embodiments, a compound described herein is administered to a mammal, including a human, to prevent the growth of beta-lactam resistant bacteria in vivo. The method according to this embodiment comprises administering a therapeutically effective amount of a penicillin-binding protein inhibitor described herein for a therapeutically effective period of time to a mammal, including a human. Preferably, the penicillin-binding protein inhibitor described herein is administered in the form of a pharmaceutical composition as described above.

In another aspect provided herein are methods of treating a bacterial infection, which method comprises administering to a subject a pharmaceutical composition comprising a compound described herein, or a pharmaceutically acceptable salt, solvate, stereoisomer, tautomer, N-oxide, dimer, or trimer thereof, and a pharmaceutically acceptable excipient. In some embodiments, the methods of treating a bacterial infection in a subject comprises administering to the subject a pharmaceutical composition as described herein. In some embodiments, the bacterial infection is an upper or lower respiratory tract infection, a urinary tract infection, an intra-abdominal infection, or a skin infection. In some embodiments, the bacterial infection is an upper or lower respiratory tract infection, a urinary tract infection, an intra-abdominal infection, or a skin infection. In some embodiments, the bacterial infection is uncomplicated or complicated urinary tract infections, uncomplicated or complicated gonorrhea, upper or lower respiratory tract infections, skin or skin structure infections, intra-abdominal infections, central nervous system infections, blood stream infections, or systemic infections.

In some embodiments, the infection that is treated or prevented is cause by a bacteria that includes Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella kingae, Moraxella catarrhalis, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebactenium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

In some embodiments, the infection that is treated or prevented is caused by a bacteria that includes Pseudomonas aeruginosa, Pseudomonas fluorescens, Stenotrophomonas maltophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter co/i, Vibrio cholerae, Vibrio parahaemolyticus, Legionella pneumophila, Listeria monocytogenes, Neisseria gonorrhoeae, Neisseria meningitidis, Moraxella catarrhalis, Bacteroides fragilis, Bacteroides vulgatus, Bacteroides ovalus, Bacteroides thetaiotaomicron, Bacteroides unformis, Bacteroides eggerthii, or Bacteroides splanchnicus.

In some embodiments, the infection that is treated or prevented is caused by a enterobacteriaceae bacteria. In some embodiments, the infection that is treated or prevented is caused by a bacteria that includes Escherichia spp, Klebsiella spp., Enterobacter spp., Citrobacter spp., Morganella spp., Proteus spp., Salmonella spp., Serratia spp., Shigella spp., or Yersinia spp.

In some embodiments, the compounds disclosed herein are useful in the treatment or prevention of infection associated with non-fermenting bacteria. In some embodiments, the compounds disclosed herein are useful in the treatment or prevention of infection associated with non-fermenting gram-negative bacteria. In some embodiments, the non-fermenting gram-negative bacteria is Pseudomonas aeruginosa, Acinetobacter spp. (A. baumannii/A. calcoaceticus), Stenotrophomonas maltophilia, Elizabethkingia spp (E. meningoseptica/E. anophelis, Burkholderia cepacia complex, Burkholderia pseudomallei, or Burkholderia mallei.

In some embodiments, the infection that is treated or prevented is tuberculosis. In some embodiments, the infection that is treated or prevented is caused by Mycobacterium tuberculosis. In some embodiments, the infection that is treated or prevented is caused by a bacteria that is a non-TB mycobacterial species. In some embodiments, the non-TB mycobacterial species is M. abscessus, M canum, M bovis, M. africanum, or M. caprae.

In some embodiments, the infection that is treated or prevented is gonorrhea. In some embodiments, the infection that is treated or prevented is caused by Neisseria gonorrhoeae.

In some embodiments, the infection that is treated or prevented is meningitis and other forms of meningococcal disease such as meningococcemia. In some embodiments, the infection that is treated or prevented is caused by Neisseria meningitidis.

In some embodiments, the infection that is treated or prevented is caused by a bacteria that is Neisseria gonorrhoeae. In some embodiments, the infection that is treated or prevented is caused by a bacteria that is Pseudomonas aeruginosa. In some embodiments, the infection that is treated or prevented is caused by a bacteria that is Acinetobacter baumannii. In some embodiments, the infection that is treated or prevented is caused by a bacteria that is Pseudomonas aeruginosa/Acinetobacter baumannii. In some embodiments, the infection that is treated or prevented is caused by a bacteria that is a carbapenem-resistant enterobacteriaceae (CRE).

In some embodiments of the methods described herein, the compound described herein is not administered with a β-lactam antibiotic. In some embodiments of the methods described herein, the compound described herein is not administered with a β-lactamase inhibitor. In some embodiments of the methods described herein, the compound described herein is not administered with a combination of a β-lactam antibiotic and a β-lactamase inhibitor.

### EXAMPLES

### General Examples for the Preparation of Compounds of Formula (I)

The starting materials and intermediates for the compounds of this invention may be prepared by the application or adaptation of the methods described below, their obvious chemical equivalents, or, for example, as described in literature such as The Science of Synthesis, Volumes 1-8. Editors E. M. Carreira et al. Thieme publishers (2001-2008). The use of protective groups may be as described in methodology compendia such as Greene's Protective Groups in Organic Synthesis, Fifth Edition. John Wiley & Sons, Inc. 2014.

Certain compounds of Formula I (Scheme 1) are prepared from the corresponding functional-group-protected boronic acid esters A by treatment with a Lewis acid in a solvent such as dichloromethane, at a temperature between −78° C. and 0° C. followed by an aqueous quench.

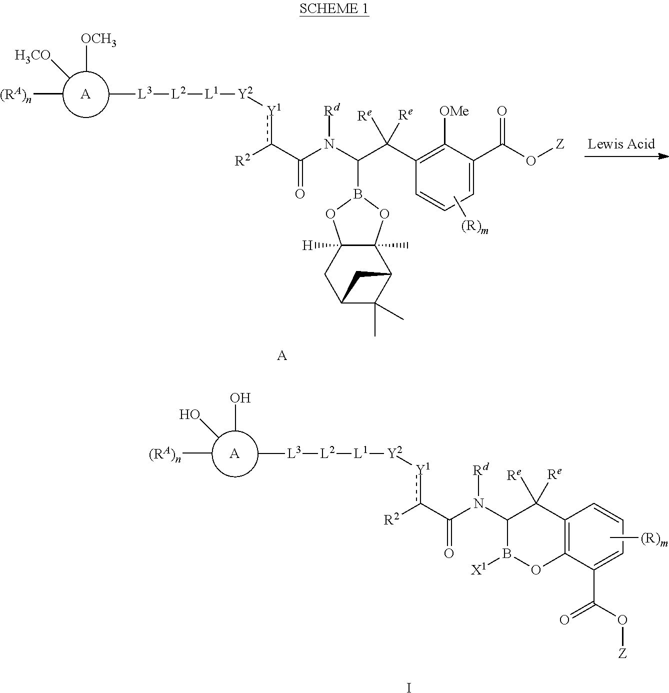

Amide intermediates A may be prepared according to the route outlined in Scheme 2. Chloro-boronates B, prepared by methods described previously (e.g. see WO2014089365), is reacted with silylamine bases such as lithium hexamethyldisilazide, and the intermediate silylamine is treated with carboxylic acids C under amide coupling conditions (such as with carbodiimide dehydrating reagents, HATU, or other coupling reagents) to provide protected amides A. Alternatively, the above silylamine intermediate is allowed to react with acid chlorides to provide A. Carboxylic acids (C) or acid chlorides (D) may be obtained from commercial sources, prepared according to known methods in the literature, or prepared by a number of different reaction sequences. Formation of the acid chloride (D) involves treatment of (C) with a chlorinating agent such as thionyl chloride, phosphorous pentachloride or oxalyl chloride, in a solvent such as dichloromethane, in the presence of a catalyst such as DMF, at around room temperature. In certain cases, DMF is also used as a co-solvent. Formation of the anhydride (E) involves treatment of (C) with a sterically hindered acid chloride or chloroformate, such as trimethylacetyl chloride or isopropylchloroformate, in an inert solvent such as dichloromethane, in the presence of a non-nucleophilic base, such as triethyl amine or diisopropylamine at room temperature or below. Formation of the activated ester (F) involves treatment of (C) with an activating reagent system such as EDCI, DCC/HOBt, HATU, BOP reagents or TBTU, in a solvent such as DMF, DMA, NMP or dichloromethane at room temperature or below (International Journal of Pharmaceutical Sciences Review and Research (2011), 8(1), 108-119).

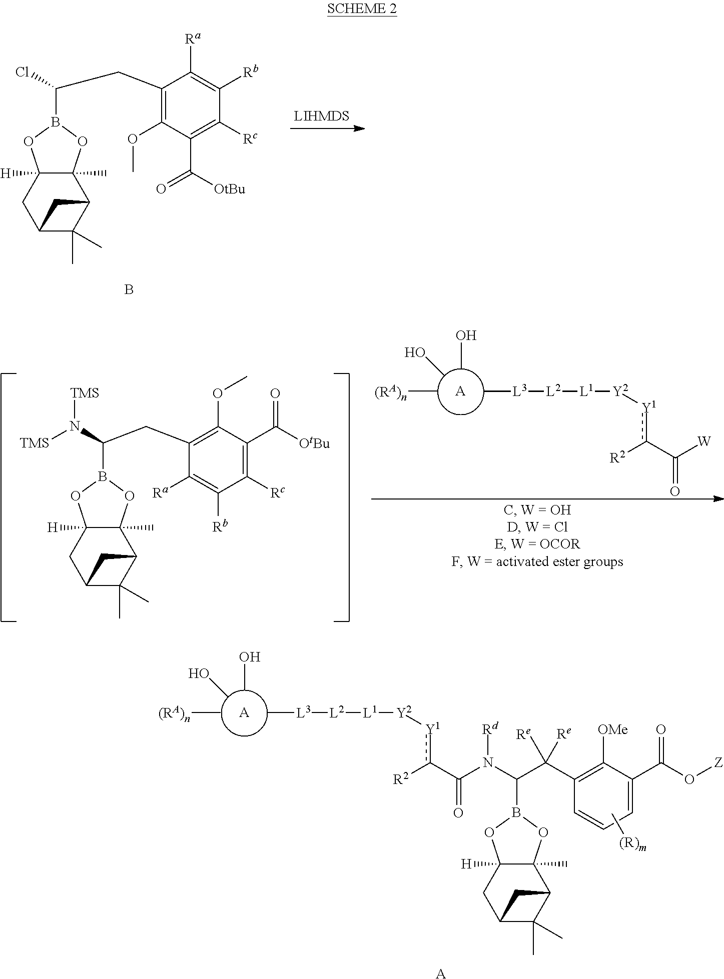

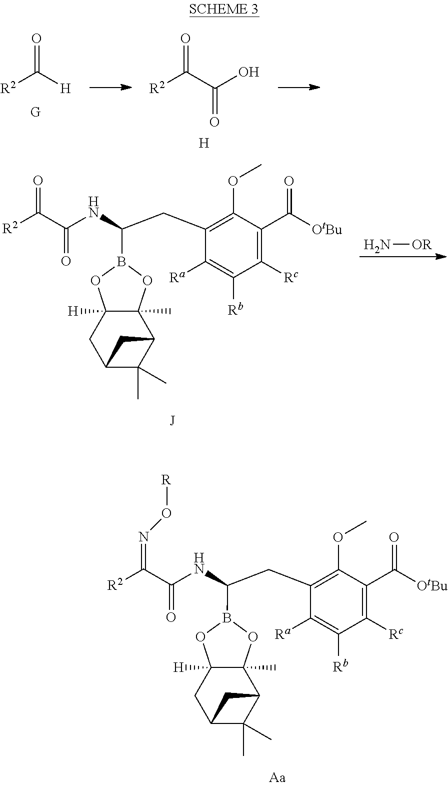

In some instances, oxime-bearing compounds Aa are prepared from the corresponding keto-amides J (Scheme 3). Using methods well-known in the art, aldehydes G are converted to keto-acids H. These keto-acids may be used in the amide coupling conditions illustrated in Scheme 2, to provide keto-amides J. The oxime functionality is then introduced by condensing J with the appropriate hydroxylamine.

Chloroboronates B may be prepared from aryl halides or aryl triflates K (X=Br, I or OTf) in the manner described in Scheme 4. Compounds K (X=Br, I or OTf) may be converted into boronic acids L by treatment with alkyl lithium reagents, for example n-butyllithium, and then quenching the intermediate aryllithium species with trialkylboronates, followed by aqueous work-up. The boronic acids L may be converted into protected boronate esters M by treatment with 1,2-diols, such as (+)-pinanediol or pinacol. Alternatively, aryl halides K may be converted to boronate esters M by transition-metal-catalyzed reaction with diboron compounds, for example bis[(+)-pinanediolato]diboron and palladium catalysts. Two sequential Matteson reactions, as described previously, provide chloroboronates B bearing a wide range of substituents R^{a}, R^{b}, and R^{c}. Another variant consists of reaction of K with chloromethyl boronate J and isopropylmagnesium chloride to provide desired intermediate N directly.

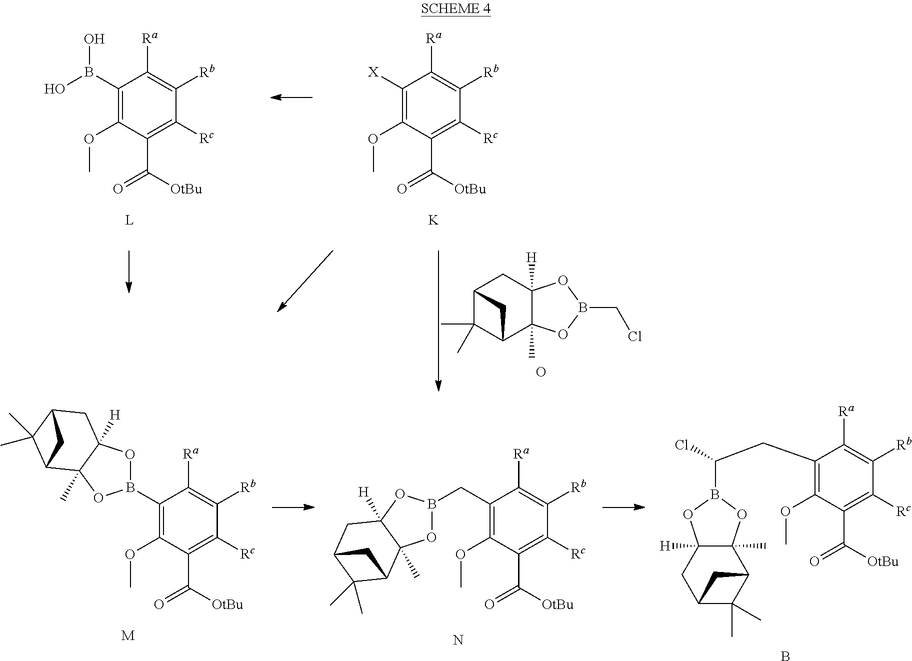

While there are common themes and strategies among the illustrative examples cited below, the selection of an appropriate reaction sequence (including protecting group requirements) is dictated by the nature and arrangement of the functionality present in the target molecule and, therefore, may involve obvious adaptations of the illustrated methods in order to be applied in a particular case.

General Method A: Deprotection with Boron Trichloride or Boron Tribromide.

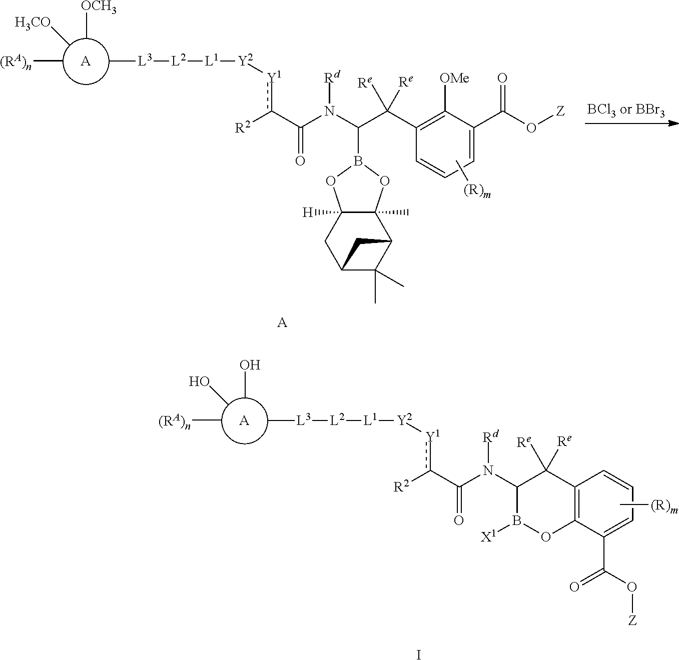

To a solution of the protected precursor A (0.4 mmol) in anhydrous DCM (15 mL) at −78° C. under argon was added dropwise BCl₃ or BBr₃ (1.0 M in DCM, 2.4-4 mL, 2.4-4 mmol, 6-10 equiv). The reaction mixture was allowed to slowly warmed to 0° C. over 1 h, and stirred between 0-5° C. for an additional 1-2 h, then quenched with water (2 mL) and methanol (20 mL), evaporated to remove DCM, washed with hexane, and concentrated to a volume of ˜4-5 mL. The crude product was purified by reverse phase preparative HPLC and dried using lyophilization to afford the product I.

General Method B: Deprotection with Aluminum Chloride.

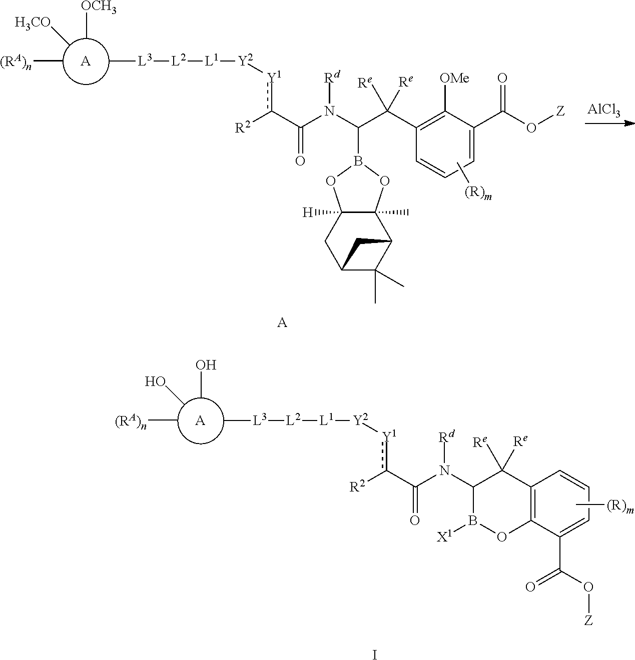

To a solution of the protected precursor A (0.4 mmol) in anhydrous DCM (15 mL) was added AlCl₃ (535 mg, 4 mmol, 10 equiv) in one portion at RT. The reaction mixture was stirred at RT for 24 h, then quenched with water (2 mL) and methanol (20 mL), evaporated to remove DCM, and washed with hexane, and concentrated to a volume of ˜4-5 mL. The crude product was purified by reverse phase preparative HPLC and dried using lyophilization to afford the product I.

### General Method C: Conversion of Chloro-Boronates to Amides.

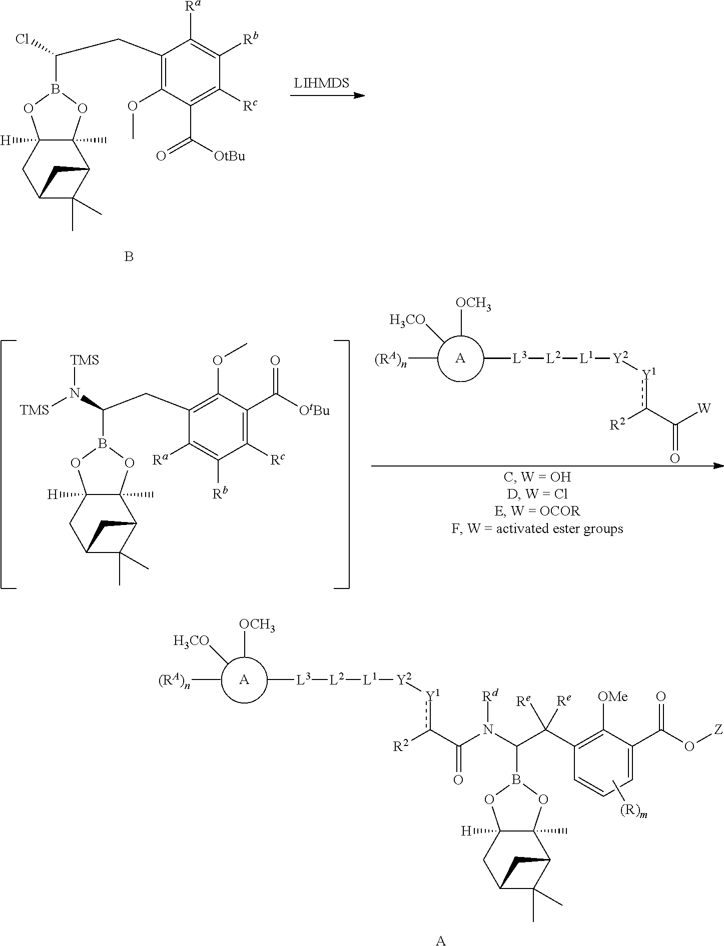

To a solution of the chloride B (4 mmol) in anhydrous THF (16 mL) was added dropwise LiHMDS (1.0 M in THF, 4.5 mL, 4.5 mmol) at −60° C. under argon. The reaction mixture was allowed to slowly warm to 0° C. over 45 min, and stirred at RT for an additional 2 h.

In a separate flask was charged the carboxylic acid C (4.2 mmol) and anhydrous DMA (20 mL), to this mixture was added HATU (1.68 g, 4.4 mmol) followed by 4-methylmorpholine (0.49 mL, 4.4 mmol). The reaction mixture was stirred at RT for 2 h, at which time the solution from the above reaction was added to the flask, and the reaction mixture was stirred at RT overnight, then diluted with EtOAc, washed with water, brine, and dried over Na₂SO₄, concentrated in vacuo to afford the crude product, which was purified by flash chromatography on silica gel (hexane-EtOAc, 20:1-1:1, or hexane-acetone, 10:1-1:1, or DCM-MeOH, 30:1-10:1) to afford the product A.

### Example 1: (3R)-3-(2-(4-(3-(2-chloro-3,4-dihydroxybenzamido)propyl)-2,3-dioxopiperazine-1-carboxamido)-2-(3-fluoro-5-hydroxypyridin-2-yl)acetamido)-2-hydroxy-3,4-dihydro-2H-benzo[e][1,2]oxaborinine-8-carboxylic acid

### Step 1. Synthesis of 5-(benzyloxy)-3-fluoropicolinaldehyde

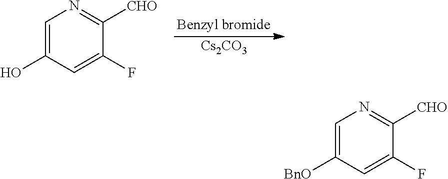

To 3-fluoro-5-hydroxypicolinaldehyde 5 g (35.5 mmol) in N,N-dimethylformamide (50 mL) was added cesium carbonate 35 g (106.4 mmol, 3 eq), followed by benzyl bromide 5.23 mL (53.2 mmol, 1.5 eq) and heated at 80° C. for 2 h. The reaction was diluted with ethyl acetate, washed with water/brine, dried over sodium sulfate and concentrated. The product was purified by flash chromatography on silica gel (20% ethyl acetate/hexanes) to give the desired product, 6.44 g, (79%) .ESI-MS m/z 232 (M+H)+.

### Step 2: Synthesis of lithium 2-(5-(benzyloxy)-3-fluoropyridin-2-yl)-2-((tert-butoxycarbonyl)amino)acetate

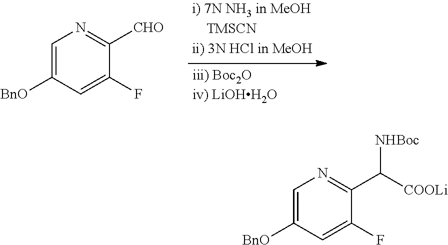

To 5-(benzyloxy)-3-fluoropicolinaldehyde 6.44 g (27.9 mmol) at 0° C. was added 7 N ammonia in methanol (160 mL), followed by trimethylsilyl cyanide 5.23 mL (41.8 mmol, 1.5 eq), stirred at 45° C. for 7 h and concentrated in vacuo. The crude product was dissolved in 3N hydrochloric acid in methanol (118 mL), stirred at 50° C. for 18 h and concentrated in vacuo to give the HCl salt. The reaction was slurried in tetrahydrofuran (120 mL) and cooled at 0° C. Triethylamine 11.6 mL (84 mmol, 3 eq) was added, followed by di-tert-butyl dicarbonate 9 g (41.4 mmol, 1.5 eq), warmed at RT for 1 h and concentrated in vacuo. The product was purified by flash chromatography on silica gel (20-30% ethyl acetate/hexanes) to give the desired product, 6.1 g. Methyl 2-((tert-butoxycarbonyl)amino)-2-(pyridin-2-yl)acetate 6.1 g (13.7 mmol) was dissolved in tetrahydrofuran (60 mL)/H₂O (60 mL), followed by lithium hydroxide monohydrate 0.92 g (21.8 mmol, 1.5 eq) and was stirred at RT for 2 h and concentrated. The product was azeotroped with toluene and dried under high vacuum for 18 h to give the title compound, 5.96 g. ESI-MS m/z 377 (M+H)+.

### Step 3: Synthesis of tert-butyl 3-((2R)-2-(2-(5-(benzyloxy)-3-fluoropyridin-2-yl)-2-((tert-butoxycarbonyl)amino)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

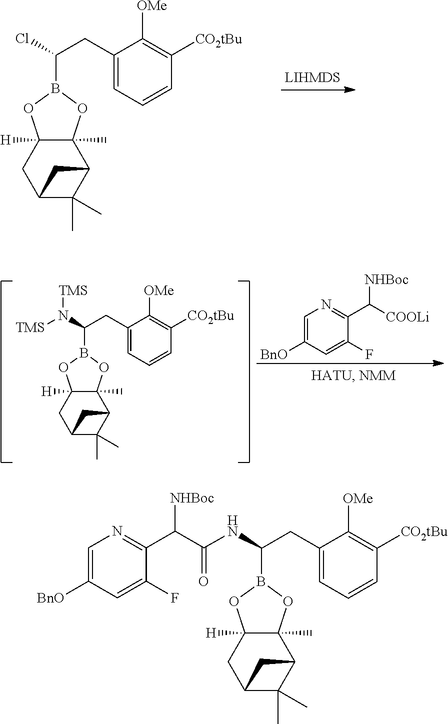

By following the General procedure C, the chloride (prepared as previous reported, WO 2014/089365) was treated with LiHMDS, and then coupled lithium 2-(5-(benzyloxy)-3-fluoropyridin-2-yl)-2-((tert-butoxycarbonyl)amino)acetate in the presence of HATU and NMM, yielding the title compound. ESI-MS m/z 788 (MH)⁺.

### Step 4: Synthesis of tert-butyl 3-((2R)-2-(2-(5-(benzyloxy)-3-fluoropyridin-2-yl)-2-(4-(3-(2-chloro-3,4-dimethoxybenzamido)propyl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

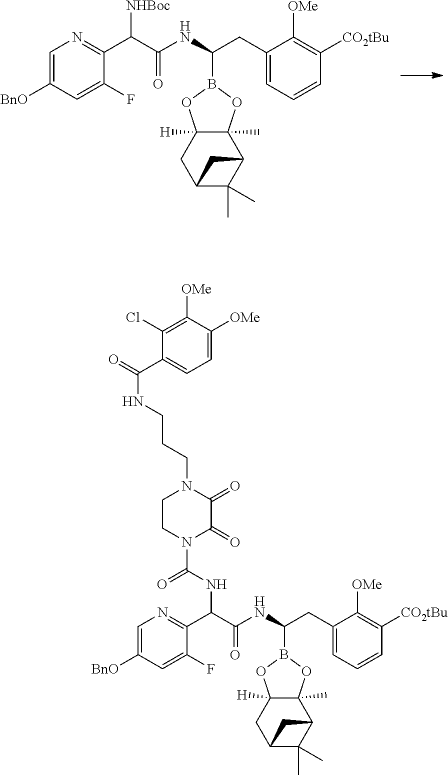 [SMILES: COc1c(C[C@H](NC(=O)C(NC(=O)OC(C)(C)C)c2ncc(OCc3ccccc3)cc2F)B2O[C@@H]3C[C@@H]4C[C@@H](C4(C)C)[C@]3(C)O2)cccc1C(=O)OC(C)(C)C ; COc1ccc(C(=O)NCCCN2CCN(C(=O)NC(C(=O)N[C@@H](Cc3cccc(C(=O)OC(C)(C)C)c3OC)B3O[C@@H]4C[C@@H]5C[C@@H](C5(C)C)[C@]4(C)O3)c3ncc(OCc4ccccc4)cc3F)C(=O)C2=O)c(Cl)c1OC]

To tert-butyl 3-((2R)-2-(2-(5-(benzyloxy)-3-fluoropyridin-2-yl)-2-((tert-butoxycarbonyl)amino)acetamido)-2-((3aS,4S,6S,7aR)-3a,5,5-trimethylhexahydro-4,6-methanobenzo[d][1,3,2]dioxaborol-2-yl)ethyl)-2-methoxybenzoate 0.785 g (0.99 mmol) at 0° C. was added 1 N hydrochloric acid in diethyl ether (19 mL) and warmed at RT for 18 h. The reaction was concentrated in vacuo to give tert-butyl 3-((2R)-2-(2-amino-2-(5-(benzyloxy)-3-fluoropyridin-2-yl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate hydrogen chloride. ESI-MS m/z 688 (MH)⁺.

To tert-butyl 3-((2R)-2-(2-amino-2-(5-(benzyloxy)-3-fluoropyridin-2-yl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate hydrogen chloride 0.3 g (0.41 (mmol) in dichloromethane (6.6 mL) at 0° C. was added N,N-diisopropylethylamine 0.22 mL (1.24 mmol, 3 eq), followed by tert-butyl (3-(4-(chlorocarbonyl)-2,3-dioxopiperazin-1-yl)propyl)carbamate 0.21 g (0.62 mmol, 1.5 eq) and the reaction was warmed at RT for 30 min. The product was quenched with water, washed with brine, dried over sodium sulfate and concentrated to give tert-butyl 3-((2R)-2-(2-(5-(benzyloxy)-3-fluoropyridin-2-yl)-2-(4-(3-((tert-butoxycarbonyl)amino)propyl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate. ESI-MS m/z 985 (MI)⁺.

The above crude product, 0.24 g (0.24 mmol) at 0° C. was added 1 N hydrochloric acid in diethyl ether (6 mL) and warmed at RT for 4 h. The reaction was concentrated in vacuo to give tert-butyl 3-((2R)-2-(2-(4-(3-aminopropyl)-2,3-dioxopiperazine-1-carboxamido)-2-(5-(benzyloxy)-3-fluoropyridin-2-yl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate hydrochloride. ESI-MS m/z 885 (MH)⁺.

To the above crude product, tert-butyl 3-((2R)-2-(2-(4-(3-aminopropyl)-2,3-dioxopiperazine-1-carboxamido)-2-(5-(benzyloxy)-3-fluoropyridin-2-yl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate hydrochloride 0.24 g (0.26 mmol) in dichloromethane (3.3 ml) was added triethylamine 0.14 mL (1.02 mmol, 3 eq), N-(3-dimethylaminopropyl)-N′-ethylcarbodiimide hydrochloride 0.054 g (0.28 mmol, 1.1 eq), 1-hydroxybenzotriazole hydrate 0.038 g (0.28 mmol, 1.1 eq), followed by 2-chloro-3,4-dimethoxybenzoic acid 0.07 g (0.31 mmol, 1.2 eq) and the reaction was stirred at RT for 18 h. The product was quenched with water, washed with brine, dried over sodium sulfate and concentrated to give the title compound. ESI-MS m/z 1083 (MH)⁺.

### Step 5: Synthesis of (3R)-3-(2-(4-(3-(2-chloro-3,4-dihydroxybenzamido)propyl)-2,3-dioxopiperazine-1-carboxamido)-2-(3-fluoro-5-hydroxypyridin-2-yl)acetamido)-2-hydroxy-3,4-dihydro-2H-benzo[e][1,2]oxaborinine-8-carboxylic acid

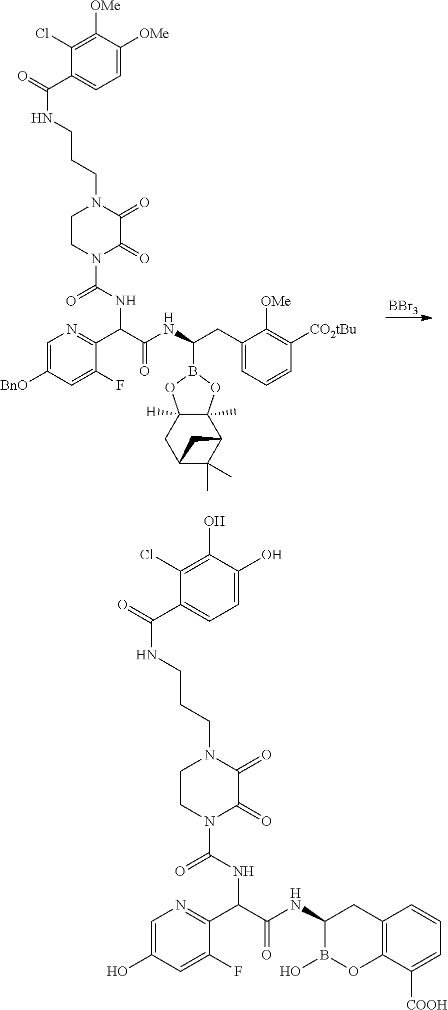

To the above crude product tert-butyl 3-((2R)-2-(2-(5-(benzyloxy)-3-fluoropyridin-2-yl)-2-(4-(3-(2-chloro-3,4-dimethoxybenzamido)propyl)-2,3-dioxopiperazine-1-carboxamido)acetamido)-2-((7aS)-5,5,7atrimethylhexahydro-4,6-methanobenzo[d][1,3,2]dioxaborol-2-yl)ethyl)-2-methoxybenzoate 0.3 g (0.27 mmol) in dichloromethane at −78° C. was added 1 N boron tribromide in dichloromethane 4 mL (4.03 mmol, 15 eq) and warmed at RT for 18 h. The reaction was quenched with water/methanol, concentrated and purified on the reverse phase HPLC (5-45% ACN:H₂O+0.1% trifluoroacetic acid to yield the title compound after lyophilization. ESI-MS m/z 743 (MH)⁺.

### Example 2: (3R)-3-(2-((S)-4-(2-(2-chloro-3,4-dihydroxybenzamido)ethyl)-6-methyl-2,3-dioxopiperazine-1-carboxamido)-2-(3-fluoro-5-hydroxypyridin-2-yl)acetamido)-2-hydroxy-3,4-dihydro-2H-benzo[e][1,2]oxaborinine-8-carboxylic acid

The title compound was prepared in a similar manner to the synthesis of Example 1, utilizing tert-butyl (S)-(2-(4-(chlorocarbonyl)-5-methyl-2,3-dioxopiperazin-1-yl)ethyl)carbamate in place of tert-butyl (3-(4-(chlorocarbonyl)-2,3-dioxopiperazin-1-yl)propyl)carbamate. ESI-MS m/z 743 (MH)⁺.

### Example 3: (3R)-3-(2-(4-(3-(2-chloro-3,4-dihydroxybenzamido)propyl)-2,3-dioxopiperazine-1-carboxamido)-2-(3-fluoro-5-hydroxypyridin-2-yl)acetamido)-7-fluoro-2-hydroxy-3,4-dihydro-2H-benzo[e][1,2]oxaborinine-8-carboxylic acid

The title compound was prepared in a similar manner to the synthesis of Example 1, utilizing tert-butyl 6-fluoro-3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate. ESI-MS m/z 761 (MH)⁺.

### Example 4: (3R)-3-(2-(2-amino-5-fluorothiazol-4-yl)-2-(4-(2-(2-chloro-3,4-dihydroxybenzamido)ethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 1, utilizing 2-(2-amino-5-fluorothiazol-4-yl)-2-((tert-butoxycarbonyl)amino)acetic acid in place of lithium 2-(5-(benzyloxy)-3-fluoropyridin-2-yl)-2-((tert-butoxycarbonyl)amino)acetate. ESI-MS m/z 752 (MH)⁺.

### Example 5: (3R)-3-(2-(4-(2-(2-chloro-6-fluoro-3,4-dihydroxybenzamido)ethyl)-2,3-dioxopiperazine-1-carboxamido)-2-(3-fluoro-5-hydroxypyridin-2-yl)acetamido)-7-fluoro-2-hydroxy-3,4-dihydro-2H-benzo[e][1,2]oxaborinine-8-carboxylic acid

The title compound was prepared in a similar manner to the synthesis of Example 1, utilizing 2-chloro-6-fluoro-3,4-dihydroxybenzoic acid in place of 2-chloro-3,4-dimethoxybenzoic acid. ESI-MS m/z 765 (MH)⁺.

### Example 6: (R)-3-((Z)-2-(2-aminothiazol-4-yl)-2-(((S)-2-carboxy-2-(2-chloro-3,4-dihydroxybenzamido)ethoxy)imino)acetamido)-2-hydroxy-3,4-dihydro-2H-benzo[e][1,2]oxaborinine-8-carboxylic acid

### Step 1. Synthesis of tert-butyl 3-((4S,11R,Z)-8-(2-((tert-butoxycarbonyl)amino)thiazol-4-yl)-4-(2-chloro-3,4-bis((4-methoxybenzyl)oxy)benzamido)-3,9-dioxo-1,1-diphenyl-11-((3aS,4S,6S,7aR)-3a,5,5-trimethylhexahydro-4,6-methanobenzo[d][1,3,2]dioxaborol-2-yl)-2,6-dioxa-7,10-diazadodec-7-en-12-yl)-2-methoxybenzoate

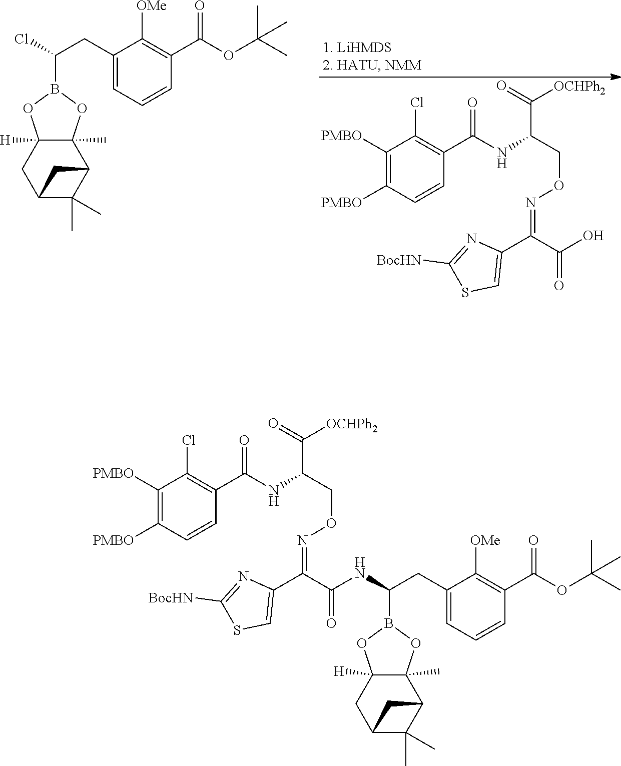

To a solution of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate (0.966 g, 2.15 mmol) in THF (10 mL) at −78° C. under argon was added LiHMDS (1.0 M in THF, 2.2 mL, 2.2 mmol) dropwise. The resulting mixture was allowed to slowly warm to room temperature overnight.

A mixture of (S,Z)-2-((3-(benzhydryloxy)-2-(2-chloro-3,4-bis((4-methoxybenzyl)oxy)benzamido)-3-oxopropoxy)imino)-2-(2-((tert-butoxycarbonyl)amino)thiazol-4-yl)acetic acid (2.08 g, 2.19 mmol), HATU (3.34 g, 8.78 mmol), NMM (1.2 mL, 10.9 mmol) in DMA (12 mL) was stirred at room temperature under argon for 1 h and then added to the reaction mixture above via cannula.

The resulting reaction mixture was stirred at room temperature for 21 h and then quenched with water, extracted with ethyl acetate (3×). The combined organic phase was dried over Na₂SO₄. After the solvent was evaporated under reduced pressure, the residue was purified by flash chromatography on silica gel (120 g column) eluted with 0 to 100% ethyl acetate/hexanes to afford 1.13 g (38.6%) of tert-butyl 3-((4S,11R,Z)-8-(2-((tert-butoxycarbonyl)amino)thiazol-4-yl)-4-(2-chloro-3,4-bis((4-methoxybenzyl)oxy)benzamido)-3,9-dioxo-1,1-diphenyl-11-((3aS,4S,6S,7aR)-3a,5,5-trimethylhexahydro-4,6-methanobenzo[d][1,3,2]dioxaborol-2-yl)-2,6-dioxa-7,10-diazadodec-7-en-12-yl)-2-methoxybenzoate as a solid. ESI-MS m/z 1362.4, 1364.4 (M+H)⁺.

### Step 2. Synthesis of (R)-3-((Z)-2-(2-aminothiazol-4-yl)-2-(((S)-2-carboxy-2-(2-chloro-3,4-dihydroxybenzamido)ethoxy)imino)acetamido)-2-hydroxy-3,4-dihydro-2H-benzo[e][1,2]oxaborinine-8-carboxylic acid

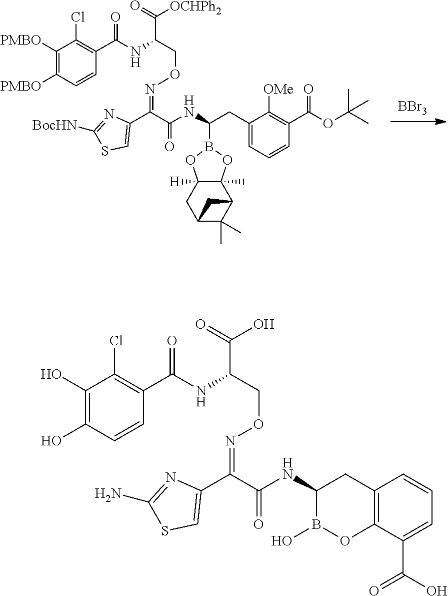

The full deprotection of tert-butyl 3-((4S,11R,Z)-8-(2-((tert-butoxycarbonyl)amino)thiazol-4-yl)-4-(2-chloro-3,4-bis((4-methoxybenzyl)oxy)benzamido)-3,9-dioxo-1,1-diphenyl-11-((3aS,4S,6S,7aR)-3a,5,5-trimethylhexahydro-4,6-methanobenzo[d][1,3,2]dioxaborol-2-yl)-2,6-dioxa-7,10-diazadodec-7-en-12-yl)-2-methoxybenzoate was carried out as described in General Method A with BBr₃ to afford (R)-3-((Z)-2-(2-aminothiazol-4-yl)-2-(((S)-2-carboxy-2-(2-chloro-3,4-dihydroxybenzamido)ethoxy)imino)acetamido)-2-hydroxy-3,4-dihydro-2H-benzo[e][1,2]oxaborinine-8-carboxylic acid. ESI-MS m/z 634.0 (M+H)⁺.

### Example 7: (3R)-3-(2-(4-carboxy-3-fluorophenyl)-2-(2-chloro-3,4-dihydroxybenzamido)acetamido)-2-hydroxy-3,4-dihydro-2H-benzo[e][1,2]oxaborinine-8-carboxylic acid

### Step 1: Synthesis of 2-(4-(tert-butoxycarbonyl)-3-fluorophenyl)-2-((tert-butoxycarbonyl)amino)acetic acid

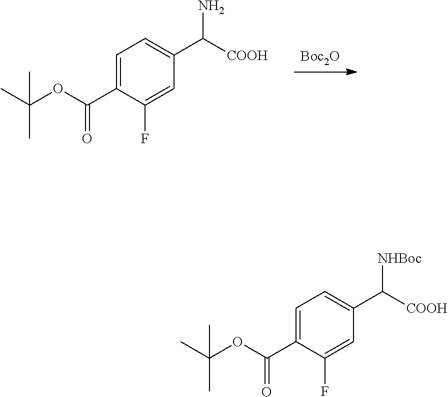 [SMILES: "Boc2O" ; CC(C)(C)OC(=O)c1ccc(C(N)C(=O)O)cc1F ; CC(C)(C)OC(=O)NC(C(=O)O)c1ccc(C(=O)OC(C)(C)C)c(F)c1]

To 2-amino-2-(4-(tert-butoxycarbonyl)-3-fluorophenyl)acetic acid 0.5 g (1.86 mmol) in tetrahydrofuran (5 mL)/water (5 mL) at 0° C. was added potassium carbonate in portions to pH 8-9, followed by boc anhydride 0.61 g (2.79 mmol, 1.5 eq) and the reaction was warmed at RT for 2 h. The organics were concentrated off and the aqueous layer was extracted with diethyl ether, acidified with 2N aqueous hydrochloric acid and extracted with dichloromethane. The organics were washed with water/brine, dried over sodium sulfate and concentrated to give the titled compound. ESI-MS m/z 392 (M+Na)⁺.

### Step 2: Synthesis of (3R)-3-(2-(4-carboxy-3-fluorophenyl)-2-(2-chloro-3,4-dihydroxybenzamido)acetamido)-2-hydroxy-3,4-dihydro-2H-benzo[e][1,2]oxaborinine-8-carboxylic acid

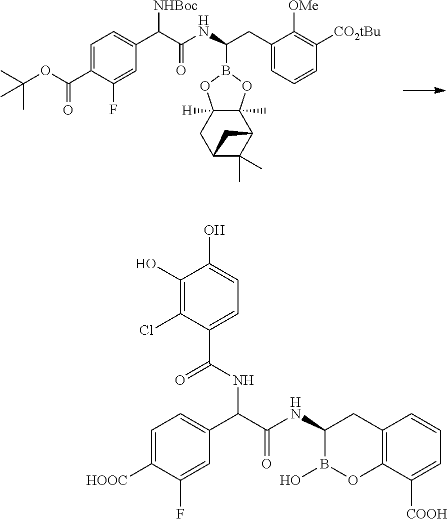 [SMILES: COc1c(C[C@H](NC(=O)C(NC(=O)OC(C)(C)C)c2ccc(C(=O)OC(C)(C)C)c(F)c2)B2O[C@@H]3C[C@@H]4C[C@@H](C4(C)C)[C@]3(C)O2)cccc1C(=O)OC(C)(C)C ; O=C(O)c1ccc(C(NC(=O)c2ccc(O)c(O)c2Cl)C(=O)N[C@H]2Cc3cccc(C(=O)O)c3OB2O)cc1F]

The title compound was prepared in a similar manner to the synthesis of Example 1, utilizing 2-chloro-3,4-dimethoxybenzoic acid in place of tert-butyl (3-(4-(chlorocarbonyl)-2,3-dioxopiperazin-1-yl)propyl)carbamate. ESI-MS m/z 573 (MH)⁺.

### Example 8: (3R)-3-(2-(2-chloro-3,4-dihydroxybenzamido)-2-(3-fluoro-5-hydroxypyridin-2-yl)acetamido)-7-fluoro-2-hydroxy-3,4-dihydro-2H-benzo[e][1,2]oxaborinine-8-carboxylic acid

The title compound was prepared in a similar manner to the synthesis of Example 1, utilizing 2-chloro-3,4-dimethoxybenzoic acid in place of tert-butyl (3-(4-(chlorocarbonyl)-2,3-dioxopiperazin-1-yl)propyl)carbamate. ESI-MS m/z 564 (MH)⁺.

### Example 9: (3R)-3-(2-(3-(2-chloro-3,4-dihydroxyphenyl)ureido)-2-(3-fluoro-5-hydroxypyridin-2-yl)acetamido)-7-fluoro-2-hydroxy-3,4-dihydro-2H-benzo[e][1,2]oxaborinine-8-carboxylic acid

The title compound was prepared in a similar manner to the synthesis of Example 1, utilizing (2-chloro-3,4-dimethoxyphenyl)carbamic chloride in place of tert-butyl (3-(4-(chlorocarbonyl)-2,3-dioxopiperazin-1-yl)propyl)carbamate. ESI-MS m/z 579 (MH)⁺.

### Example 10: (3R)-3-(2-(2-chloro-6-fluoro-3,4-dihydroxybenzamido)-2-(3-fluoro-5-hydroxypyridin-2-yl)acetamido)-7-fluoro-2-hydroxy-3,4-dihydro-2H-benzo[e][1,2]oxaborinine-8-carboxylic acid

The title compound was prepared in a similar manner to the synthesis of Example 1, utilizing 2-chloro-6-fluoro-3,4-dihydroxybenzoic acid in place of tert-butyl (3-(4-(chlorocarbonyl)-2,3-dioxopiperazin-1-yl)propyl)carbamate. ESI-MS m/z 582 (MH)⁺.

### Example 11: (3R)-3-(2-(4-carboxy-3-fluorophenyl)-2-(4-(2-(2-chloro-3,4-dihydroxybenzamido)ethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 4, utilizing 2-(4-(tert-butoxycarbonyl)-3-fluorophenyl)-2-((tert-butoxycarbonyl)amino)acetic acid in place of 2-(2-amino-5-fluorothiazol-4-yl)-2-((tert-butoxycarbonyl)amino)acetic acid. ESI-MS m/z 774 (MH)⁺.

### Example 12: (R)-3-((R)-2-(2-chloro-6-fluoro-3,4-dihydroxybenzamido)-3,3-dimethylbutanamido)-7-fluoro-2-hydroxy-3,4-dihydro-2H-benzo[e][1,2]oxaborinine-8-carboxylic acid

The title compound was prepared in a similar manner to the synthesis of Example 10, utilizing (R)-2-((tert-butoxycarbonyl)amino)-3,3-dimethylbutanoic acid in place of lithium 2-(5-(benzyloxy)-3-fluoropyridin-2-yl)-2-((tert-butoxycarbonyl)amino)acetate. ESI-MS m/z 527 (MH)⁺.

### Example 13: (R)-3-((R)-2-(4-(3-aminopropyl)-2,3-dioxopiperazine-1-carboxamido)-2-(3-fluoro-5-hydroxypyridin-2-yl)acetamido)-2-hydroxy-3,4-dihydro-2H-benzo[e][1,2]oxaborinine-8-carboxylic acid

The title compound was prepared in a similar manner to the synthesis of Example 1. ESI-MS m/z 573 (MH)⁺.

### Example 14: (R)-3-((R)-2-((S)-4-(2-aminoethyl)-6-methyl-2,3-dioxopiperazine-1-carboxamido)-2-(3-fluoro-5-hydroxypyridin-2-yl)acetamido)-2-hydroxy-3,4-dihydro-2H-benzo[e][1,2]oxaborinine-8-carboxylic acid

The title compound was prepared in a similar manner to the synthesis of Example 1, utilizing tert-butyl (S)-(2-(4-(chlorocarbonyl)-5-methyl-2,3-dioxopiperazin-1-yl)ethyl)carbamate in place of tert-butyl (3-(4-(chlorocarbonyl)-2,3-dioxopiperazin-1-yl)propyl)carbamate. ESI-MS m/z 573 (MH)⁺.

### Example 15: (3R)-3-(2-(4-carboxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of tert-butyl(4-fluoro-2-methoxyphenoxy)dimethylsilane

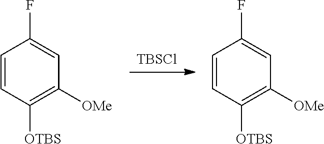 [SMILES: COc1cc(F)ccc1O[Si](C)(C)C(C)(C)C ; "TBSCl" ; COc1cc(F)ccc1O[Si](C)(C)C(C)(C)C]

To a solution of 4-fluoro-2-methoxyphenol (5.68 g, 40 mmol) in DCM (100 mL) was added TEA (11.2 mL, 80 mmol), 4-DMAP (488 mg, 4 mmol) followed by TBSCl (7.5 g, 49.8 mmol). The reaction mixture was stirred at RT for overnight, then re-cooled to 0° C., Boc₂O (36.7 g, 168 mmol) was added. The reaction mixture was stirred at RT overnight, washed with aqueous NaHCO₃, dried over Na₂SO₄, and concentrated. The crude product was purified by flash chromatography on silica gel (hexane-EtOAc, 50:1-10:1) to afford the title compound, 10 g. ESI-MS m/z 257 (M+H)⁺.

### Step 2. Synthesis of tert-butyl 3-((tert-butyldimethylsilyl)oxy)-6-fluoro-2-methoxybenzoate

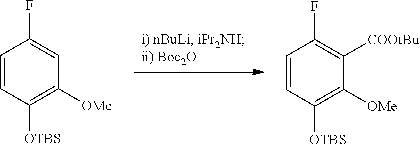

To a solution of diisopropylamine (6.6 mL, 46.8 mmol) in anhydrous THF (120 mL) at −65° C. was added nBuLi (2.5 M, 18.72 mL, 46.8 mmol) dropwise under argon. The reaction mixture was stirred between −60° C.-−55° C. for 20 min. To this reaction mixture was added the above product (10 g, 39 mmol) in THF (15 mL) dropwise, stirred for 1 h, then Boc₂O (28.19 g, 129 mmol) was added. The reaction mixture was slowly warmed up to RT, and stirred at RT overnight, quenched with water, extracted with ethyl acetate. The organic extracts were washed with brine, dried over Na₂SO₄, and concentrated. The crude product was purified by flash chromatography on silica gel (DCM-hexane, 1:20-1:1) to afford the title compound (8 g), which was contaminated with some by product and Boc₂O. ESI-MS m/z 357 (M+H)⁺.

### Step 3. Synthesis of tert-butyl 6-fluoro-3-hydroxy-2-methoxybenzoate

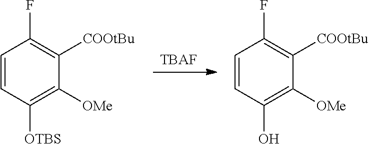

To a solution of the above product (8 g, 22.5 mmol) in THF (150 mL) was added TBAF (1.0 M, 50 mL, 50 mmol), the reaction was stirred at RT for 1.5 h, diluted with EtOAc, washed with saturated aqueous NaHCO₃, brine, dried over Na₂SO₄, and concentrated. The crude product was purified by flash chromatography on silica gel (hexane-EtOAc, 40:1-2:1) to afford the title compound, 1.4 g, ESI-MS m/z 243 (M+H)⁺, and O-Boc product, 2. 6 g, ESI-MS m/z 343 (M+H)⁺. The O-Boc product (2.6 g) was treated with excess piperidine in DCM at RT overnight to afford additional 1.4 g of the title compound after purification by flash chromatography.

### Step 4. Synthesis of tert-butyl 6-fluoro-2-methoxy-3-(((trifluoromethyl)sulfonyl)oxy)benzoate

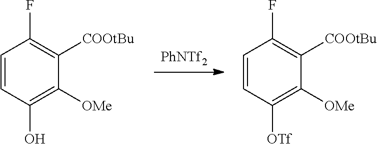

To a solution of the above product (1.4 g, 5.8 mmol) in DCM (50 mL) was added PhNTf₂ (2.9 g, 8.12 mmol), TEA (2.03 mL, 14.5 mmol) and 4-DMAP (71 mg, 0.58 mmol). The reaction mixture was stirred at RT overnight, washed with saturated aqueous NaHCO₃, dried over Na₂SO₄, and concentrated. The crude product was purified by flash chromatography on silica gel (hexane-DCM, 10:1-1:4) to afford the title compound, 1.9 g. ESI-MS m/z 375 (M+Na)⁺.

### Step 5. Synthesis of tert-butyl 6-fluoro-2-methoxy-3-((3aS,4S,6S,7aR)-3a,5,5-trimethylhexahydro-4,6-methanobenzo[d][1,3,2]dioxaborol-2-yl)benzoate

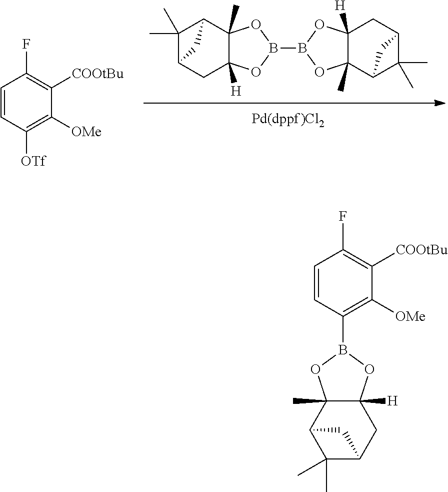

To the above product (3.85 g, 10.3 mmol) in dry DMF (35 mL) was added bis[(+)-pinanediolato]diboron (5.7 g, 15.9 mmol), KOAc (3.1 g, 31.6 mmol) and Pd(dppf)Cl₂.DCM (430 mg, 0.53 mmol). The reaction mixture was stirred at 90-100° C. overnight, added water, and extracted with diethyl ether. The ether extracts were washed with water, brine, dried over Na₂SO₄, and concentrated. The crude product was purified by flash chromatography on silica gel (hexane-DCM, 10:1-1:10) to afford the title compound, 2.4 g. ESI-MS m/z 831 (2M+Na)⁺.

### Step 6. Synthesis of tert-butyl 6-fluoro-2-methoxy-3-(((3aS,4S,6S,7aR)-3a,5,5-trimethylhexahydro-4,6-methanobenzo[d][1,3,2]dioxaborol-2-yl)methyl)benzoate

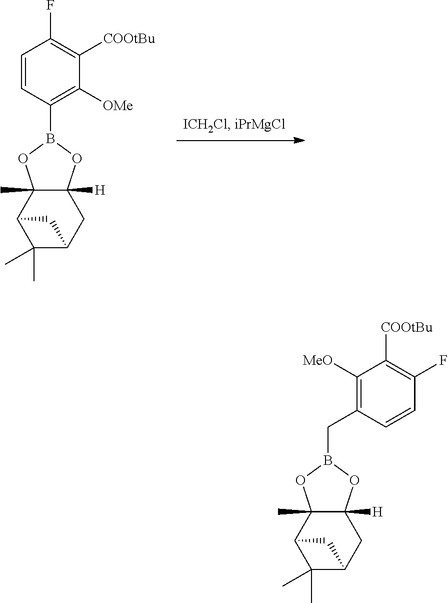

To a solution of chloroiodomethane (3.2 mL, 43.9 mmol) in THF (70 mL) at −78° C. was added dropwise under Argon isopropyl magnesium chloride lithium chloride complex solution (1.3 M in THF, 16.8 mL, 21.8 mmol) over 20 min. The resulting solution was stirred at −78° C. for 45 min, then a solution of the above product (2.38 g, 5.89 mmol) in THF (9 mL) was added slowly over 20 min. After the addition was completed, the reaction mixture was stirred for 1.5 h. To this solution was added ZnCl₂ solution (1.0 M in ether, 6.4 mL, 6.4 mmol) dropwise, and stirring continued for 15 min after the addition was completed. The cold bath was removed, the reaction mixture was stirred at RT overnight, cooled to −30° C., diluted with diethyl ether, washed with aqueous NH₄C1, water and brine, dried over Na₂SO₄, and concentrated, purified by flash chromatography on silica gel (hexane-EtOAc, 20:1-5:1) to give the title compound, 2.24 g. ESI-MS m/z 441 (M+Na)+.

### Step 7. Synthesis of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

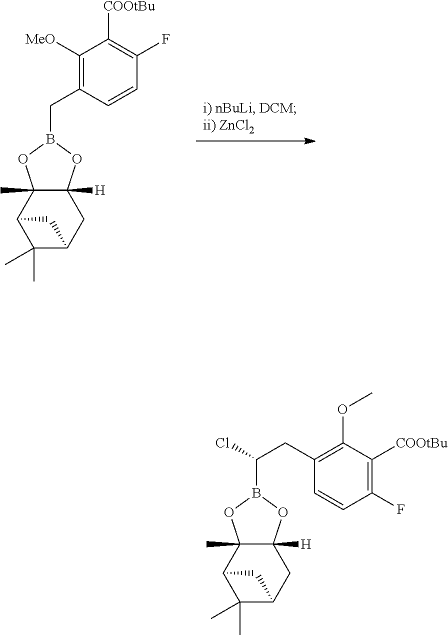

To a cooled (−100° C. MeOH/N₂) solution of DCM (0.82 mL, 12.8 mmol) in THF (15 mL) was added dropwise, down the side of the flask nBuLi (2.5 M in hexane, 3.06 mL, 7.65 mmol) over 20 min. The resulting mixture was stirred for 45 min, then a solution of the above product (2.24 g, 5.36 mmol) in THF (8 mL) was added slowly down the side of the flask over 20 min, and stirring continued for 45 min after the addition was completed. To the resulting mixture was added dropwise a solution of ZnCl₂ (1.0 M in ether, 7.3 mL, 7.3 mmol) over 5 min. After 15 min, the methanol/N₂ bath was replaced with a dry ice/acetone bath (−10° C.), and stirring continued for 1.5 h. The reaction mixture was diluted with diethyl ether and washed with aqueous NH₄Cl, water and brine, dried over Na₂SO₄, and concentrated. The crude product was purified by flash chromatography on silica gel (hexane-EtOAc, 20:1-4:1) to afford the title compound, 2.1 g. ESI-MS m/z 489 (M+Na)⁺.

### Step 8. Synthesis of 2-(4-(tert-butoxycarbonyl)phenyl)-2-(4-ethyl-2,3-dioxopiperazine-1-carboxamido)acetic acid

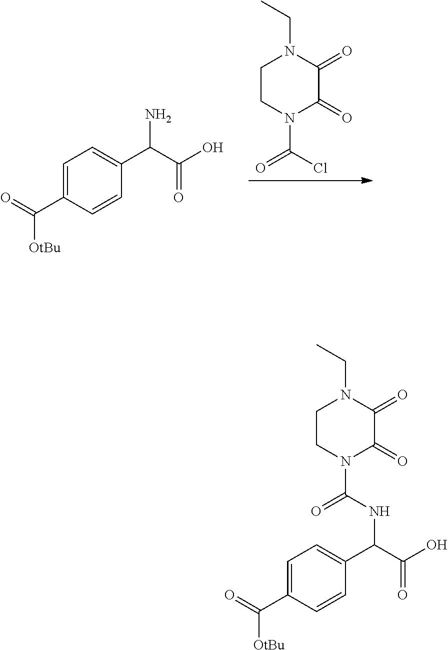 [SMILES: CC(C)(C)OC(=O)c1ccc(C(N)C(=O)O)cc1 ; CCN1CCN(C(=O)Cl)C(=O)C1=O ; CCN1CCN(C(=O)NC(C(=O)O)c2ccc(C(=O)OC(C)(C)C)cc2)C(=O)C1=O]

To 2-amino-2-(4-(tert-butoxycarbonyl)phenyl)acetic acid (5.02 g, 20 mmol) (which was prepared from tert-butyl 4-acetylbenzoate by following the reported procedures: WO 2013051597) in THF (75 mL) and water (75 mL) was added LiOH.H₂O (882 mg, 21 mmol), followed by saturated aqueous NaHCO₃ (50 mL) and then 4-ethyl-2,3-dioxopiperazine-1-carbonyl chloride (6.14 g, 30 mmol) in THF (50 mL) at 0° C. The reaction mixture was stirred at RT for 1.5 h, concentrated in vacuo, extracted with Et₂O. The aqueous was acidified with 1 N HCl to pH=2-3, the solid was collected by filtration, dried in vacuo, yielding the title compound, 8 g. ESIMS m/z 420 (MH)⁺.

### Step 9. Synthesis of (3R)-3-(2-(2-aminothiazol-4-yl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

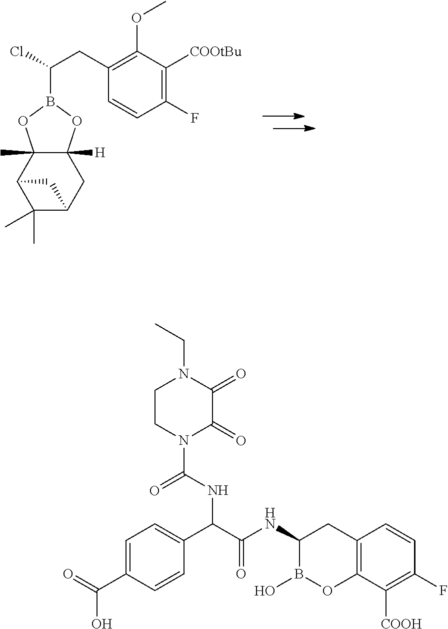

The title compound was prepared from the above chloride from Step 7, and the carboxylic acid from Step 8 by following the General coupling method C, and utilizing TMSI in the final deprotection reaction in DCM at RT for 2 h, the title compound was prepared. ESIMS m/z 571 (M+1)⁺.

### Example 16: (R)-3-((R)-2-(4-(3-aminopropyl)-2,3-dioxopiperazine-1-carboxamido)-2-(3-fluoro-5-hydroxypyridin-2-yl)acetamido)-7-fluoro-2-hydroxy-3,4-dihydro-2H-benzo[e][1,2]oxaborinine-8-carboxylic acid

The title compound was prepared in a similar manner to the synthesis of Example 13, utilizing tert-butyl 6-fluoro-3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate. ESI-MS m/z 591 (MH)⁺.

### Example 17: (3R)-3-(2-(3-acetamido-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(4-methoxy-3-nitrophenyl)acetic acid

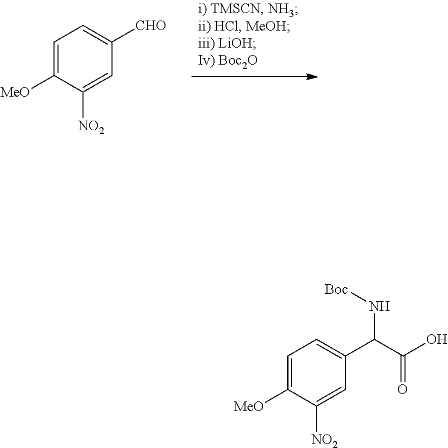 [SMILES: COc1ccc(C=O)cc1[N+](=O)[O-] ; COc1ccc(C(NC(=O)OC(C)(C)C)C(=O)O)cc1[N+](=O)[O-]]

### Step 1a

To 4-methoxy-3-nitrobenzaldehyde (4.16 g, 23 mmol) was added 7 N NH₃ in methanol (150 mL) at 0° C. followed by TMSCN (4.3 mL, 33.6 mmol). The reaction mixture was stirred at 0° C. for 15 min, then heated at 45° C. for 5 h, then concentrated in vacuo.

### Step 1b

The crude product was dissolved in methanol (150 mL) and 4 N HCl in dioxane (150 mL), heated at 50° C. overnight, then concentrated in vacuo.

### Step 1c

This crude product in THF (150 mL), and water (150 mL) was treated with LiOH.H₂O (3.87 g, 92 mmol) at RT for 1.5 h, then neutralized with 1 N HCl to pH=8-9.

### Step 1d

To the above mixture was added by saturated aqueous NaHCO₃ (60 mL) and then Boc₂O (8.3 g, 38 mmol) in THF (50 mL) at 0° C. The reaction mixture was stirred at RT for 3 h, concentrated in vacuo, extracted with Et₂O. The aqueous was acidified with 1 N HCl to pH=2-3, extracted with EtOAc, the combined organic extracts were dried over Na₂SO₄, concentrated in vacuo to yield the title compound, 6 g. ESIMS m/z 349 (M+Na)⁺.

Step 2. Synthesis of tert-butyl 3-((2R)-2-(2-((tert-butoxycarbonyl)amino)-2-(4-methoxy-3-nitrophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate.

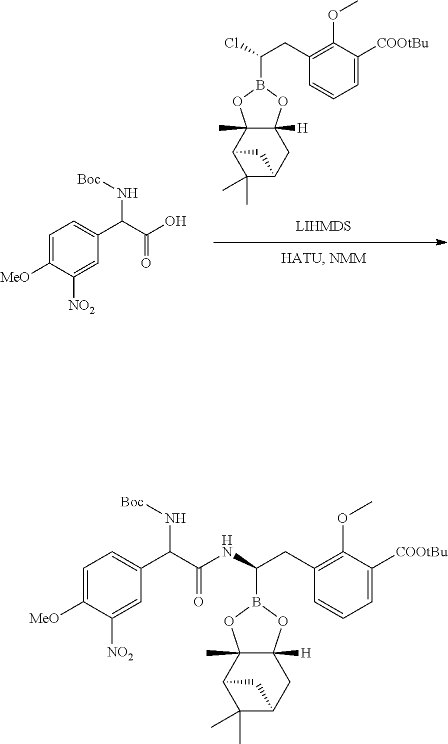

By following the General coupling method C, the title compound was prepared from the above acid and the chloride intermediate (prepared as previous reported, WO 2014/089365). ESI-MS m/z 738 (M+H)⁺.

### Step 3. Synthesis of tert-butyl 3-((2R)-2-(2-amino-2-(4-methoxy-3-nitrophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

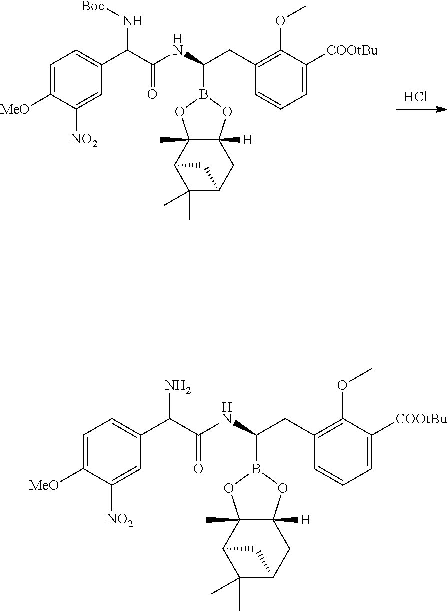

The above compound (7 g, 9.5 mmol) was treated with 1 N HCl in Et₂O (300 mL, 300 mmol) at RT overnight, added 4 N HCl in dioxane (25 mL, 100 mmol), and the reaction mixture was heated at 45° C.-50° C. for 1 h, added more 4 N HCl in dioxane (25 mL, 100 mmol), heated at 45° C.-50° C. for an additional 3.5 h, then concentrated in vacuo, yielding the crude product which used without further purification for the next step. ESI-MS m/z 638 (M+H)⁺.

### Step 4. Synthesis of tert-butyl 3-((2R)-2-(2-(4-ethyl-2,3-dioxopiperazine-1-carboxamido)-2-(4-methoxy-3-nitrophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

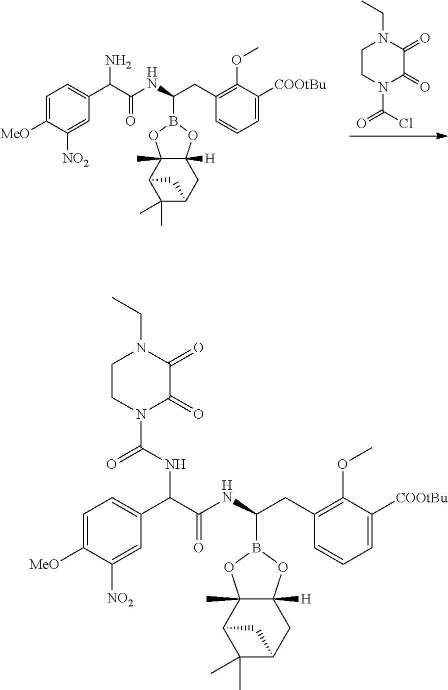

To the above crude product (3 g, 4.45 mmol) in DCM (120 mL) was added iPr₂NEt (2.6 mL, 14.9 mmol) at 0° C., followed by 4-ethyl-2,3-dioxopiperazine-1-carbonyl chloride (910 mg, 4.45 mmol). The reaction mixture was stirred at RT for 1.5 h, washed with water, brine, dried over Na₂SO₄, concentrated in vacuo to yield the crude product which was used without further purification for the next step. ESI-MS m/z 806 (M+H)⁺.

### Step 5. Synthesis of tert-butyl 3-((2R)-2-(2-(3-acetamido-4-methoxyphenyl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

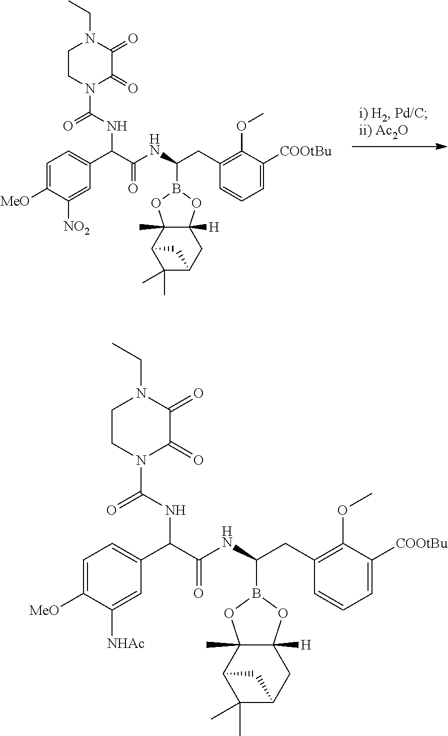

To the above compound (644 mg, 0.8 mmol) in MeOH (20 mL) was hydrogenated in the presence of 10% Pd/C (166 mg) using a hydrogen balloon at rt for 5 h, then filtered and concentrated in vacuo. The crude product was dissolved in DCM (20 mL), half (10 mL, 0.4 mmol) of this was used for the acetylation by treatment with Ac₂O (182 mg, 1.8 mmol) in the presence of iPr₂NEt (0.54 mL, 3.1 mmol) to yield the crude product which was used without further purification for the next step. ESI-MS m/z 818 (M+H)⁺.

### Step 6. Synthesis of (3R)-3-(2-(3-acetamido-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

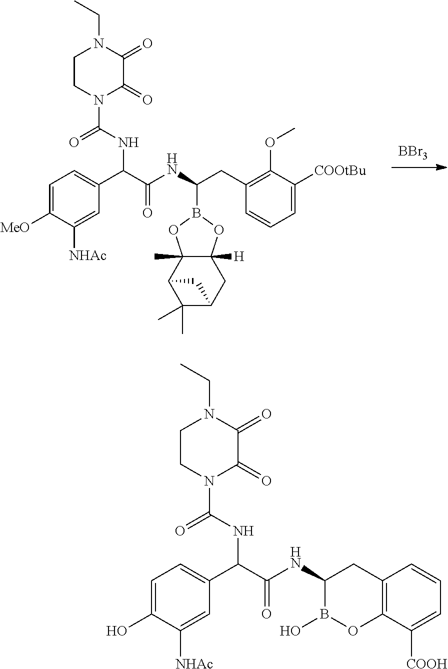

By following the general deprotection and purification method, the above compound was treated with excess BBr₃ at RT overnight to yield the title compound after reversed phase HPLC purification. ESI-MS m/z 582 (M+1)+.

### Example 18: (3R)-3-(2-(2-amino-5-fluorothiazol-4-yl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 4, utilizing 4-ethyl-2,3-dioxopiperazine-1-carbonyl chloride in place of tert-butyl (2-(4-(chlorocarbonyl)-2,3-dioxopiperazin-1-yl)ethyl)carbamate. ESI-MS m/z 567 (MH)⁺.

### Example 19: 6-((S)-2-(((R)-8-carboxy-2-hydroxy-3,4-dihydro-2H-benzo[e][1,2]oxaborinin-3-yl)amino)-1-(4-ethyl-2,3-dioxopiperazine-1-carboxamido)-2-oxoethyl)-2-chloro-5-fluoronicotinic acid

### Step 1: Synthesis of tert-butyl 6-(1-((tert-butoxycarbonyl)amino)-2-ethoxy-2-oxoethyl)-2-chloro-5-fluoronicotinate

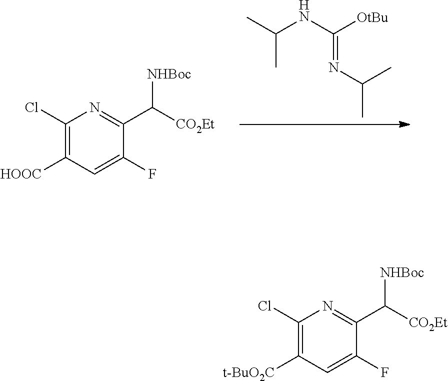

To 6-(1-((tert-butoxycarbonyl)amino)-2-ethoxy-2-oxoethyl)-2-chloro-5-fluoronicotinic acid 2 g (5.32 mmol) was added tert-butyl N,N′-diisopropylcarbamimidate 7.18 mL (32 mmol, 6 eq), followed by tert-butanol (30 mL) and the reaction was stirred at RT for 18 h. The white solid was filtered off and the filtrate was concentrated. The residue was dissolved in ethyl acetate, washed with water/brine, dried over sodium sulfate and concentrated. The product was purified by flash chromatography on silica gel (20% ethyl acetate/hexanes) to give the desired product, 1.86 g, (81%). ESI-MS m/z 455 (M+Na)⁺.

### Step 2: Synthesis of tert-butyl 6-(2-(((R)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-((tert-butoxycarbonyl)amino)-2-oxoethyl)-2-chloro-5-fluoronicotinate

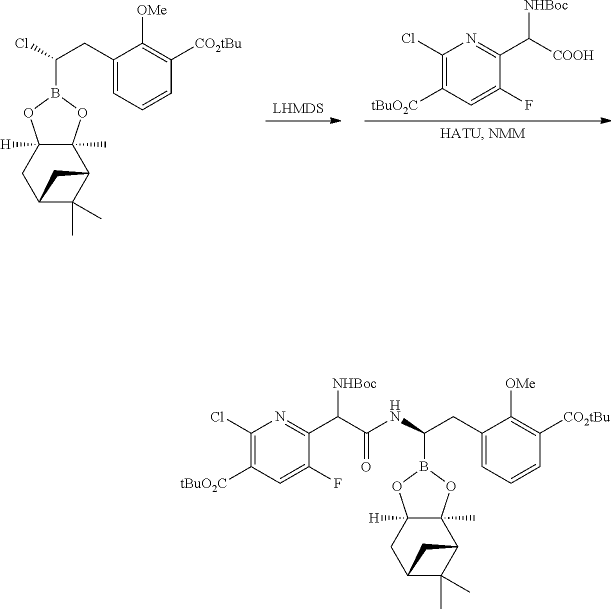

By following the General Procedure C, the chloride (prepared as previous reported, WO 2014/089365) was treated with LiHMDS, and then coupled 2-(5-(tert-butoxycarbonyl)-6-chloro-3-fluoropyridin-2-yl)-2-((tert-butoxycarbonyl)amino)acetic acid in the presence of HATU and NMM, yielding the title compound. ESI-MS m/z 816 (MH)⁺.

### Step 3: Synthesis of 6-((S)-2-(((R)-8-carboxy-2-hydroxy-3,4-dihydro-2H-benzo[e][1,2]oxaborinin-3-yl)amino)-1-(4-ethyl-2,3-dioxopiperazine-1-carboxamido)-2-oxoethyl)-2-chloro-5-fluoronicotinic acid

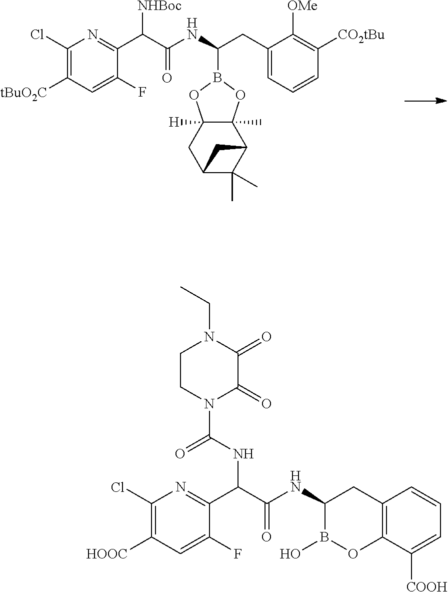 [SMILES: COc1c(C[C@H](NC(=O)C(NC(=O)OC(C)(C)C)c2nc(Cl)c(C(=O)OC(C)(C)C)cc2F)B2O[C@@H]3C[C@@H]4C[C@@H](C4(C)C)[C@]3(C)O2)cccc1C(=O)OC(C)(C)C ; CCN1CCN(C(=O)NC(C(=O)N[C@H]2Cc3cccc(C(=O)O)c3OB2O)c2nc(Cl)c(C(=O)O)cc2F)C(=O)C1=O]

The title compound was prepared in a similar manner to the synthesis of Example 1, utilizing 4-ethyl-2,3-dioxopiperazine-1-carbonyl chloride in place of tert-butyl (3-(4-(chlorocarbonyl)-2,3-dioxopiperazin-1-yl)propyl)carbamate. ESI-MS m/z 606 (MH)⁺.

### Example 20: 6-(2-(((R)-8-carboxy-2-hydroxy-3,4-dihydro-2H-benzo[e][1,2]oxaborinin-3-yl)amino)-1-(4-ethyl-2,3-dioxopiperazine-1-carboxamido)-2-oxoethyl)-5-fluoronicotinic acid

### Step 1: Synthesis of tert-butyl 6-(2-(((R)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-(4-ethyl-2,3-dioxopiperazine-1-carboxamido)-2-oxoethyl)-5-fluoronicotinate

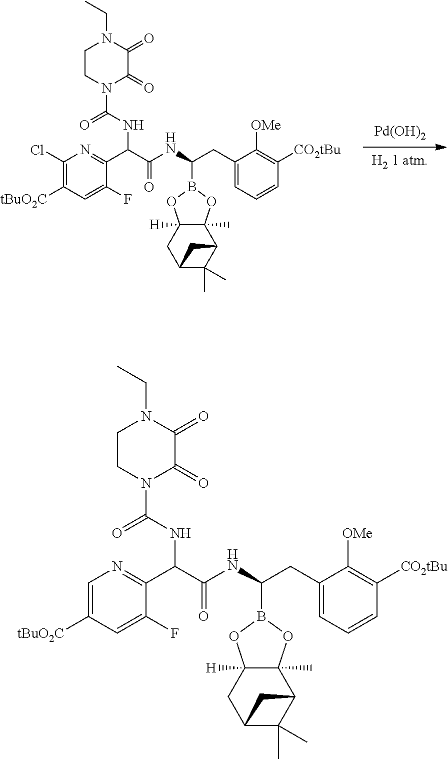 [SMILES: CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2nc(Cl)c(C(=O)OC(C)(C)C)cc2F)C(=O)C1=O ; "Pd(OH)2" ; "H2 1 atm." ; CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ncc(C(=O)OC(C)(C)C)cc2F)C(=O)C1=O]

To tert-butyl 6-(2-(((R)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-(4-ethyl-2,3-dioxopiperazine-1-carboxamido)-2-oxoethyl)-2-chloro-5-fluoronicotinate 0.28 g (0.3 mmol) in methanol (3 mL) was added a spatula tip of palladium hydroxide on carbon under an atmosphere of argon. The reaction was stirred under a hydrogen balloon at RT for 18 h and filtered through a pad of celite. The filtrate was concentrated and dried under high vacuum to give the title compound. ESI-MS m/z 850 (MH)⁺.

### Step 2: Synthesis of 6-(2-(((R)-8-carboxy-2-hydroxy-3,4-dihydro-2H-benzo[e][1,2]oxaborinin-3-yl)amino)-1-(4-ethyl-2,3-dioxopiperazine-1-carboxamido)-2-oxoethyl)-5-fluoronicotinic acid

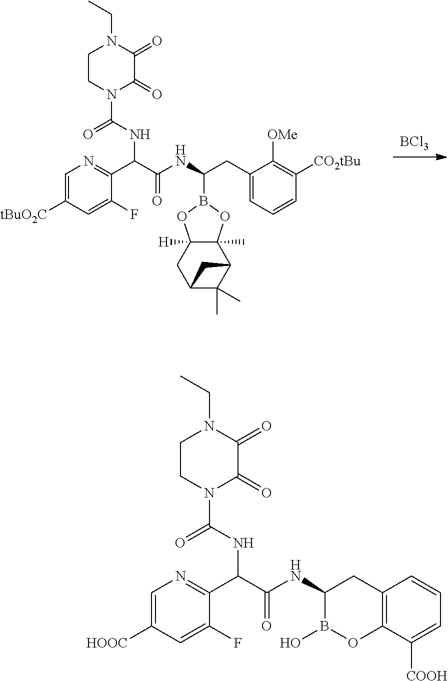 [SMILES: CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ncc(C(=O)OC(C)(C)C)cc2F)C(=O)C1=O ; "BCl3" ; CCN1CCN(C(=O)NC(C(=O)N[C@H]2Cc3cccc(C(=O)O)c3OB2O)c2ncc(C(=O)O)cc2F)C(=O)C1=O]

The title compound was prepared in a similar manner to the synthesis of Example 1, utilizing 1 M boron trichloride in dichloromethane in place of 1 M boron tribromide in dichloromethane. ESI-MS m/z 572 (MH).

### Example 21: (3R)-3-(2-(4-ethyl-2,3-dioxopiperazine-1-carboxamido)-2-(4-hydroxy-3-(N-(methylsulfonyl)methylsulfonamido)phenyl)acetamido)-2-hydroxy-3,4-dihydro-2H-benzo[e][1,2]oxaborinine-8-carboxylic acid

In a similar manner to the synthesis of Example 17, the hydrogenation product (0.4 mmol) of Step 5 in DCM (10 mL) was treated with methanesulfonyl chloride (106 mg, 0.92 mmol) in the presence of iPr₂NEt (0.22 mL, 1.26 mmol) and a catalytic amount of 4-DMAP at RT for 1.5 h to yield the crude bis-mesylated product which was treated with excess BBr₃ at RT overnight, affording the title compound after reversed phase HPLC purification. ESI-MS m/z 696 (M+1)⁺.

### Example 22: (3R)-3-(2-(4-ethyl-2,3-dioxopiperazine-1-carboxamido)-2-(4-hydroxy-3-(methylsulfonamido)phenyl)acetamido)-2-hydroxy-3,4-dihydro-2H-benzo[e][1,2]oxaborinine-8-carboxylic acid

In a similar manner to the synthesis of Example 17, the hydrogenation product (0.8 mmol) of Step 5 in DCM (8 mL) was treated with MsCl (0.25 ml, 3.2 mmol) in the presence of pyridine (0.8 mL, 9.9 mmol) at RT for 1.5 h to yield the crude mesylated product which was treated with excess BBr₃ at RT overnight, affording the title compound after reversed phase HPLC purification. ESI-MS m/z 618 (M+1)⁺.

### Example 23: (3R)-3-(2-(2-chloro-3,4-dihydroxyphenyl)-2-(3-(methylsulfonyl)-2-oxoimidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 17 utilizing 2-chloro-3,4-dimethoxybenzaldehyde in place of 4-methoxy-3-nitrobenzaldehyde in Step 1, utilizing in Step 2 the 7-F chloride intermediate (prepared from Step 7 of Example 15), and utilizing 3-(methylsulfonyl)-2-oxoimidazolidine-1-carbonyl chloride in place of 4-ethyl-2,3-dioxopiperazine-1-carbonyl chloride in Step 4, the title compound was prepared. ESI-MS m/z 615/617 (MH/MH+2)⁺.

### Example 24: (3R)-3-(2-(2-amino-5-fluorothiazol-4-yl)-2-(4-(2-aminoethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 16, utilizing 2-(2-amino-5-fluorothiazol-4-yl)-2-((tert-butoxycarbonyl)amino)acetic acid in place of lithium 2-(5-(benzyloxy)-3-fluoropyridin-2-yl)-2-((tert-butoxycarbonyl)amino)acetate. ESI-MS m/z 582 (MH)⁺.

### Example 25: (3R)-3-(2-amino-2-(2-chloro-6-fluoro-3,4-dihydroxyphenyl)acetamido)-7-fluoro-2-hydroxy-3,4-dihydro-2H-benzo[e][1,2]oxaborinine-8-carboxylic acid

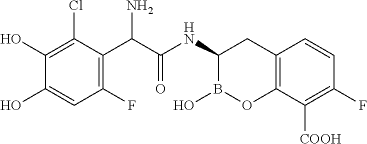 [SMILES: NC(C(=O)N[C@H]1Cc2ccc(F)c(C(=O)O)c2OB1O)c1c(F)cc(O)c(O)c1Cl]

### Step 1. Synthesis of 2-fluoro-5-hydroxy-4-methoxybenzaldehyde

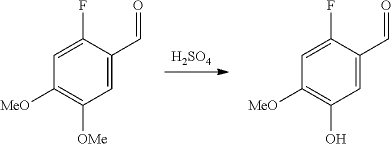

2-Fluoro-4,5-dimethoxybenzaldehyde (7.5 g, 40.8 mmol) in concentrated sulfuric acid (30 mL) was heated at 90° C. for 5 h, then cooled to RT, poured into ice-water, extracted with EtOAc. The organic extracts were combined, washed with brine, dried over Na₂SO₄, concentrated in vacuo. To the residue was added a mixed solvent of hexane-EtOAc (1:1, 50 mL). The solid was collected by filtration to afford the title compound, 4.6 g. ESI-MS m/z 171 (MH)⁺.

### Step 2. Synthesis of 2-chloro-6-fluoro-3-hydroxy-4-methoxybenzaldehyde

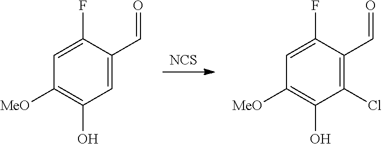 [SMILES: COc1cc(F)c(C=O)cc1O ; "NCS" ; COc1cc(F)c(C=O)c(Cl)c1O]

To the above product (4.6 g, 27.1 mmol) in DMF (50 mL) was added NCS (4.81 g, 36 mmol). The reaction mixture was stirred at RT overnight, diluted with diethylether, washed with water, brine, dried over Na₂SO₄, concentrated in vacuo to afford the crude product, 5.2 g, which was used directly for the next step without further purification. ESI-MS m/z 205/207 (MH/MH+2)⁺.

### Step 3. Synthesis of 2-chloro-6-fluoro-3,4-dimethoxybenzaldehyde

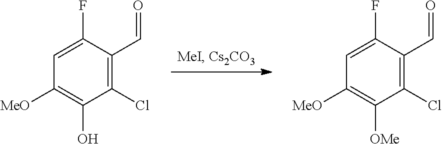

To the above crude product (5.2 g, 25.5 mmol) in DMF (60 mL) was added Cs₂CO₃ (20.8 g, 63.8 mmol) followed by iodomethane (5 mL, 80 mmol). The reaction mixture was stirred at RT overnight, diluted with diethylether, washed with water, brine, dried over Na₂SO₄, concentrated in vacuo. The residue was purified by flash chromatography on silica gel (hexane-EtOAc, 10:1-2:1) to afford the title compound, 3 g. ESI-MS m/z 219/221 (MH/MH+2)⁺.

### Step 4. Synthesis of tert-butyl 3-((2R)-2-(2-((tert-butoxycarbonyl)amino)-2-(2-chloro-6-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

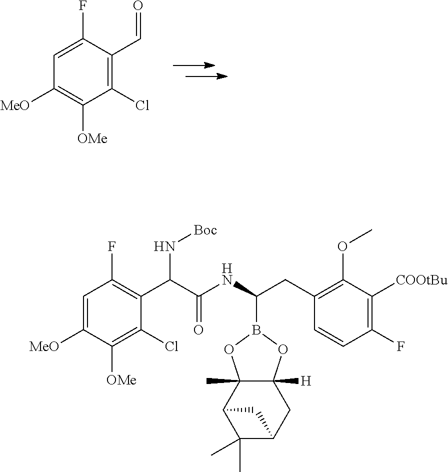

In a similar manner to the synthesis of Example 17 utilizing the above aldehyde in place of 4-methoxy-3-nitrobenzaldehyde in Step 1, utilizing in Step 2 the 7-F chloride intermediate (described in Step 7 of Example 15), the title compound was prepared. ESI-MS m/z 776/778 (MH/MH+2)+. Step 5. Synthesis of (3R)-3-(2-amino-2-(2-chloro-6-fluoro-3,4-dihydroxyphenyl)acetamido)-7-fluoro-2-hydroxy-3,4-dihydro-2H-benzo[e][1,2]oxaborinine-8-carboxylic acid.

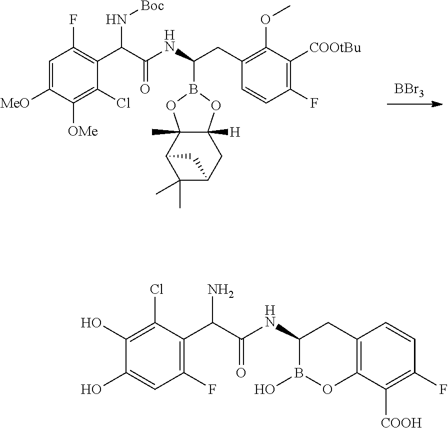

The above product was treated with excess BBr₃ at RT overnight, affording the title compound after reversed phase HPLC purification. ESI-MS 443/445 (MH/MH+2)⁺.

### Example 26: (R)-3-((R)-2-(4-(2-aminoethyl)-2,3-dioxopiperazine-1-carboxamido)-2-(2-chloro-6-fluoro-3,4-dihydroxyphenyl)acetamido)-7-fluoro-2-hydroxy-3,4-dihydro-2H-benzo[e][1,2]oxaborinine-8-carboxylic acid

### Step 1. Synthesis of tert-butyl (2-(4-(chlorocarbonyl)-2,3-dioxopiperazin-1-yl)ethyl)carbamate

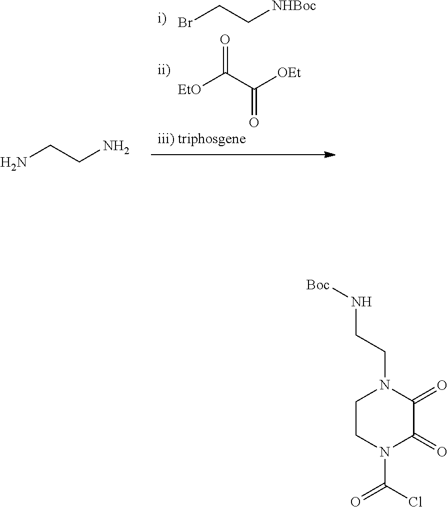

### Step 1a

To ethylenediamine (22.5 g, 25 mL, 375 mmol) was added 2-(Boc-amino)ethyl bromide (12.1 g, 54 mmol) in portions over 20 min, stirred at RT for an additional 5 h. To the reaction mixture was added diethyl ether (50 mL), the ether layer was separated, and the residue was extracted one more time with diethyl ether (50 mL). The ether extracts were combined, concentrated and dried in vacuo.

### Step 1b

To the EtOH (100 mL) solution of this crude product (10.8 g, 53.2 mmol) was added diethyl oxalate (7.84 mL, 57.7 mmol). The reaction mixture was stirred at reflux for 18 h, then concentrated in vacuo, the residue was purified by flash chromatography on silica gel (DCM-MeOH, 20:1-4:1) to afford the product, 8.45 g. ESI-MS m/z 258 (M+H)⁺.

### Step 1c

To the above product (2.57 g, 10 mmol) in THF (25 mL) and DCM (12 mL) at −15° C. was added chlorotrimethylsilane (1.4 mL, 11 mmol), followed by triethylamine (1.7 mL, 12.1 mmol). The reaction mixture was stirred between −15° C.-0° C. for 1 h, then triphosgene (1.2 g, 4 mmol) in THF (6 mL) was added dropwise to the reaction mixture. After addition was complete, the reaction mixture was warmed up to RT over 30 min, stirred for an additional 1 h, and the solid was filtered off and washed with THF. The filtrate was concentrated in vacuo to give the crude title compound which was used directly for the next step without further purification.

### Step 2. Synthesis of (R)-3-((R)-2-(4-(2-aminoethyl)-2,3-dioxopiperazine-1-carboxamido)-2-(2-chloro-6-fluoro-3,4-dihydroxyphenyl)acetamido)-7-fluoro-2-hydroxy-3,4-dihydro-2H-benzo[e][1,2]oxaborinine-8-carboxylic acid

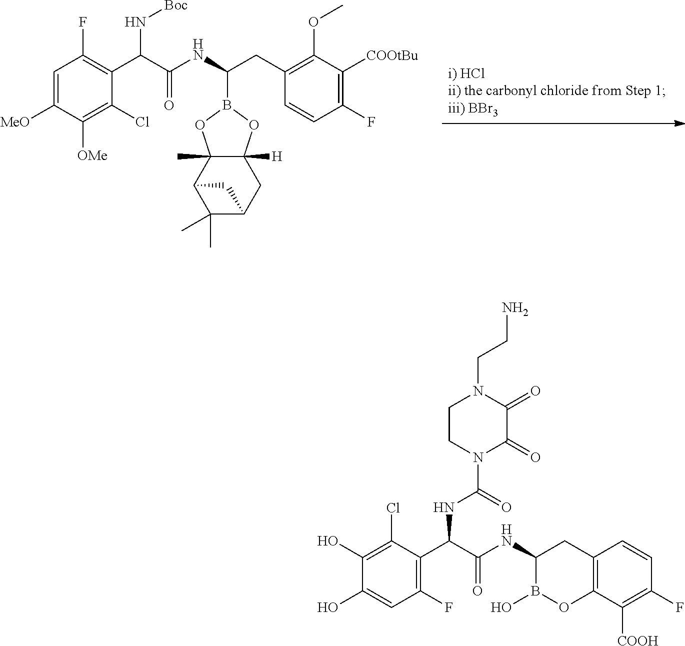

By following the same procedures described in Step 3, Step 4 and Step 6 of Example 17, utilizing tert-butyl (2-(4-(chlorocarbonyl)-2,3-dioxopiperazin-1-yl)ethyl)carbamate in place of 4-ethyl-2,3-dioxopiperazine-1-carbonyl chloride in Step 4, the intermediate from Step 4 of Example 25 was converted to the title compound after reversed phase HPLC purification, isolated as the first eluting peak. ESI-MS m/z 626/628 (MH/MH+2)⁺.

### Example 27: (R)-3-((R)-2-(4-(3-aminopropyl)-2,3-dioxopiperazine-1-carboxamido)-2-(2-chloro-6-fluoro-3,4-dihydroxyphenyl)acetamido)-7-fluoro-2-hydroxy-3,4-dihydro-2H-benzo[e][1,2]oxaborinine-8-carboxylic acid

### Step 1. Synthesis of tert-butyl (3-(4-(chlorocarbonyl)-2,3-dioxopiperazin-1-yl)propyl)carbamate

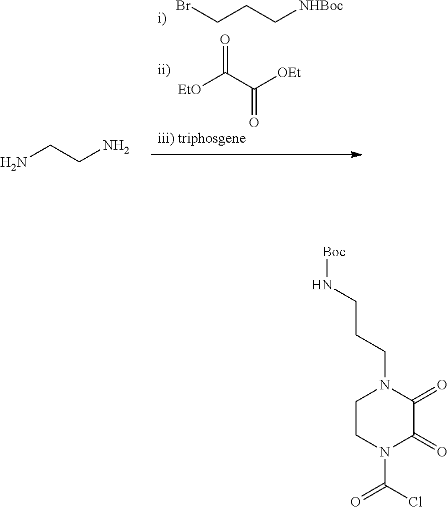

By following the same procedures as described for the synthesis of tert-butyl (2-(4-(chlorocarbonyl)-2,3-dioxopiperazin-1-yl)ethyl)carbamate in Step 1 of Example 26, the title compound was prepared from tert-butyl (3-bromopropyl)carbamate.

### Step 2. Synthesis of (R)-3-((R)-2-(4-(3-aminopropyl)-2,3-dioxopiperazine-1-carboxamido)-2-(2-chloro-6-fluoro-3,4-dihydroxyphenyl)acetamido)-7-fluoro-2-hydroxy-3,4-dihydro-2H-benzo[e][1,2]oxaborinine-8-carboxylic acid

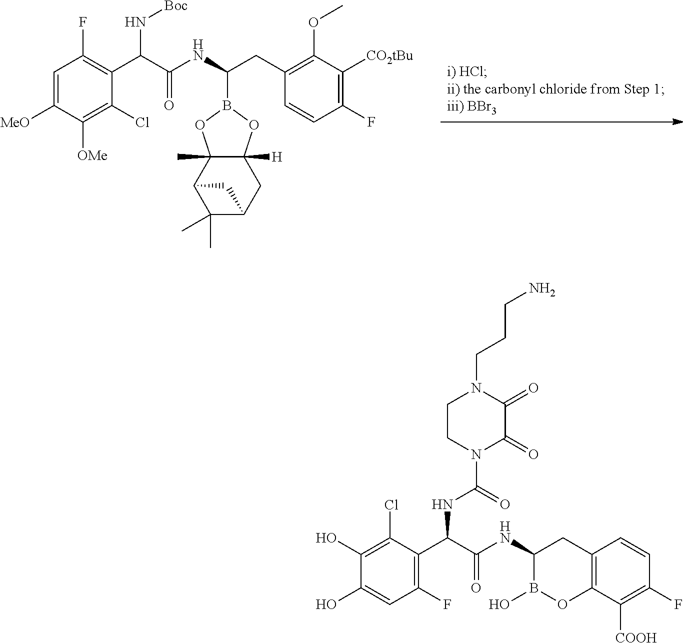

In a similar manner to the synthesis of Example 26, utilizing tert-butyl (3-(4-(chlorocarbonyl)-2,3-dioxopiperazin-1-yl)propyl)carbamate from Step 1 in place of tert-butyl (2-(4-(chlorocarbonyl)-2,3-dioxopiperazin-1-yl)ethyl)carbamate, the title compound was prepared after reversed phase HPLC purification, isolated as the first eluting peak. ESI-MS m/z 640/642 (MH/MH+2)⁺.

### Example 28: (R)-3-((S)-2-(4-(3-aminopropyl)-2,3-dioxopiperazine-1-carboxamido)-2-(2-chloro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 23, utilizing tert-butyl (3-(4-(chlorocarbonyl)-2,3-dioxopiperazin-1-yl)propyl)carbamate (described in Step 1 of Example 27) in place of 3-(methylsulfonyl)-2-oxoimidazolidine-1-carbonyl chloride in Step 4, the title compound was prepared after reversed phase HPLC purification, isolated as the second eluting peak. ESI-MS m/z 622/624 (MH/MH+2)⁺.

### Example 29: (3R)-3-(2-(3-amino-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The hydrogenation product as described in Step 5 of Example 17 was treated with excess BBr₃ at RT overnight, affording the title compound after reversed phase HPLC purification. ESI-MS m/z 540 (M+1)⁺.

### Example 30: (3R)-3-(2-(4-ethyl-2,3-dioxopiperazine-1-carboxamido)-2-(4-hydroxy-3-(sulfamoylamino)phenyl)acetamido)-2-hydroxy-3,4-dihydro-2H-benzo[e][1,2]oxaborinine-8-carboxylic acid

To a DCM (2.5 mL) solution of chlorosulfonylisocynate (68.3 mg, 0.48 mmol) at 0° C. under Argon was added tBuOH (36.4 mg, 0.48 mmol) in DCM (0.5 mL). After stirring for 30 min at 0° C., this solution was added to a solution of the hydrogenation product from Step 5 of Example 17 (279 mg, 0.36 mmol) and iPr₂NEt (0.19 mL, 1.07 mmol) at 0° C., the reaction mixture was then warmed up to RT, stirred for 3 h, washed with water, brine, dried over Na₂SO₄, concentrated in vacuo. This crude product was treated with excess BBr₃ at RT overnight, affording the title compound after reversed phase HPLC purification. ESI-MS m/z 619 (M+1).

### Example 31: (3R)-3-(2-(4-carboxy-3-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

By following the same procedure of Step 8 of Example 15, the General Method C using the chloride intermediate (prepared as previous reported, WO 2014/089365), and final deprotection with TMSI as described in Step 9 of Example 15, 2-amino-2-(4-(tert-butoxycarbonyl)-3-fluorophenyl)acetic acid (which was prepared from tert-butyl 4-acetyl-2-fluorobenzoate by following the reported procedures: WO2013051597) was converted to the title compound. ESIMS m/z 571 (M+1)+.

### Example 32: (R)-3-((R)-2-amino-2-(4-((fluorosulfonyl)oxy)phenyl)acetamido)-7-fluoro-2-hydroxy-3,4-dihydro-2H-benzo[e][1,2]oxaborinine-8-carboxylic acid

### Step 1: Synthesis of tert-butyl 3-((R)-2-((R)-2-((tert-butoxycarbonyl)amino)-2-(4-((fluorosulfonyl)oxy)phenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

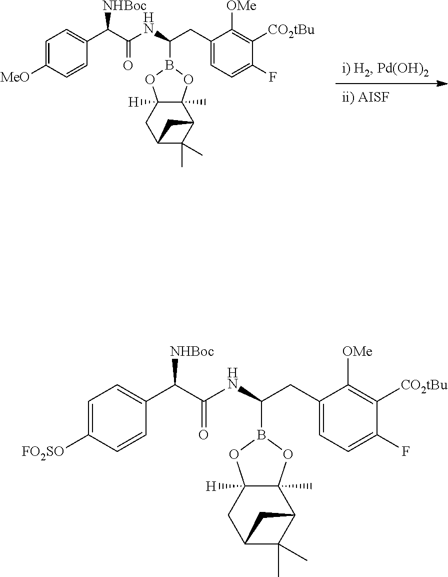

To tert-butyl 3-((R)-2-((R)-2-(4-(benzyloxy)phenyl)-2-((tert-butoxycarbonyl)amino)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 0.45 g (0.57 mmol) in methanol was added a spatula tip of palladium hydroxide on carbon under an atmosphere of argon. The reaction was stirred under a hydrogen balloon at RT for 2 h and filtered through a pad of celite. The filtrate was concentrated and dried under high vacuum to give tert-butyl 3-((R)-2-((R)-2-((tert-butoxycarbonyl)amino)-2-(4-hydroxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate. ESI-MS m/z 697 (MH)+.

To the above crude product, tert-butyl 3-((R)-2-((R)-2-((tert-butoxycarbonyl)amino)-2-(4-hydroxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate, 0.423 g (0.61 mmol) in tetrahydrofuran (8.5 mL) was added 1,8-diazabicyclo[5.4.0]undec-7-ene 0.2 mL (1.34 mmol, 2.2 eq), followed by [4-(acetylamino)phenyl]-imidodisulfuryl difluoride, 0.23 g (0.73 mmol, 1.2 eq) and stirred at RT for 30 minutes. The reaction was diluted with ethyl acetate, washed with water, dried over sodium sulfate and concentrated. The product was purified by flash chromatography on silica gel (40% ethyl acetate/hexanes) to give the title compound, 0.22 g, (47%). ESI-MS m/z 801 (M+Na)⁺.

### Step 2: Synthesis of (R)-3-((R)-2-amino-2-(4-((fluorosulfonyl)oxy)phenyl)acetamido)-7-fluoro-2-hydroxy-3,4-dihydro-2H-benzo[e][1,2]oxaborinine-8-carboxylic acid

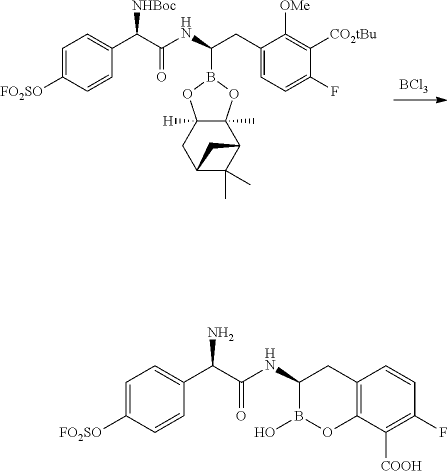

The title compound was prepared in a similar manner to the synthesis of Example 1, utilizing 1 M boron trichloride in dichloromethane in place of 1M boron tribromide in dichloromethane. ESI-MS m/z 457 (MH)⁺

### Example 33: 6-(2-(((R)-8-carboxy-7-fluoro-2-hydroxy-3,4-dihydro-2H-benzo[e][1,2]oxaborinin-3-yl)amino)-1-(4-ethyl-2,3-dioxopiperazine-1-carboxamido)-2-oxoethyl)-5-fluoronicotinic acid

The title compound was prepared in a similar manner to the synthesis of Example 20, utilizing tert-butyl 6-fluoro-3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate. ESI-MS m/z 590 (MH)⁺.

### Example 34: (R)-3-((R)-2-(2-chloro-6-fluoro-3,4-dihydroxyphenyl)-2-(2-oxoimidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 26, utilizing 2-oxoimidazolidine-1-carbonyl chloride in place of tert-butyl (2-(4-(chlorocarbonyl)-2,3-dioxopiperazin-1-yl)ethyl)carbamate, the title compound was prepared after reversed phase HPLC purification, isolated as the first eluting peak. ESI-MS m/z 555/557 (MH/MH+2)⁺.

### Example 35: (R)-3-((R)-2-(2-chloro-6-fluoro-3,4-dihydroxyphenyl)-2-(3-(methylsulfonyl)-2-oxoimidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 26, utilizing 3-(methylsulfonyl)-2-oxoimidazolidine-1-carbonyl chloride in place of tert-butyl (2-(4-(chlorocarbonyl)-2,3-dioxopiperazin-1-yl)ethyl)carbamate, the title compound was prepared after reversed phase HPLC purification, isolated as the first eluting peak. ESI-MS m/z 633/635 (MH/MH+2)⁺.

### Example 36: (3R)-3-(2-(2,6-difluoro-3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of tert-butyl 3-((2R)-2-(2-amino-2-(2,6-difluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

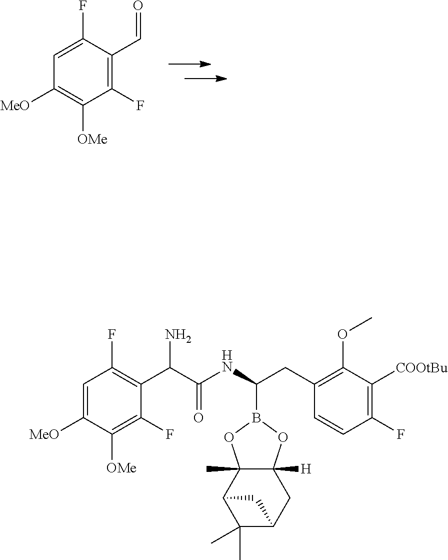

By following the procedures described in Step 1-3 of Example 17, utilizing 2,6-difluoro-3,4-dimethoxybenzaldehyde in place of 4-methoxy-3-nitrobenzaldehyde in Step 1, utilizing in Step 2 the 7-F chloride intermediate (described in Step 7 of Example 15), the title compound was prepared. ESI-MS m/z 677 (M+1)⁺.

### Step 2. Synthesis of (R)-3-((R)-2-(4-(3-aminopropyl)-2,3-dioxopiperazine-1-carboxamido)-2-(2-chloro-6-fluoro-3,4-dihydroxyphenyl)acetamido)-7-fluoro-2-hydroxy-3,4-dihydro-2H-benzo[e][1,2]oxaborinine-8-carboxylic acid

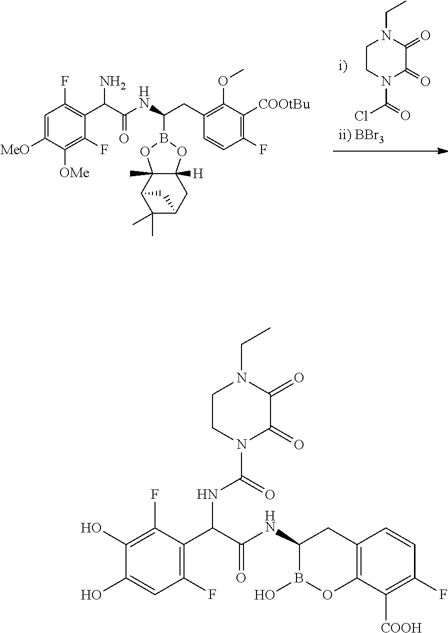

By following the same procedure of Step 4 of Example 17 by acylation with 4-ethyl-2,3-dioxopiperazine-1-carbonyl chloride, followed by deprotection with excess BBr₃, the title compound was prepared from the above amine intermediate. ESI-MS m/z 595 (M+1)⁺.

### Example 37: (3R)-3-(2-(2,6-difluoro-3,4-dihydroxyphenyl)-2-(4-(2-fluoroethyl)-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of 4-(2-fluoroethyl)-2,3-dioxopiperazine-1-carbonyl chloride

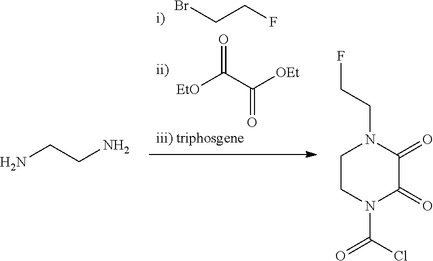

By following the same procedures as described for the synthesis of tert-butyl (2-(4-(chlorocarbonyl)-2,3-dioxopiperazin-1-yl)ethyl)carbamate in Step 1 of Example 26, the title compound was prepared from 1-bromo-2-fluoroethane.

### Step 2. Synthesis of (3R)-3-(2-(2,6-difluoro-3,4-dihydroxyphenyl)-2-(4-(2-fluoroethyl)-2,3-dioxopiperazine-1-carboxamido)acetamido)-7-fluoro-2-hydroxy-3,4-dihydro-2H-benzo[e][1,2]oxaborinine-8-carboxylic acid

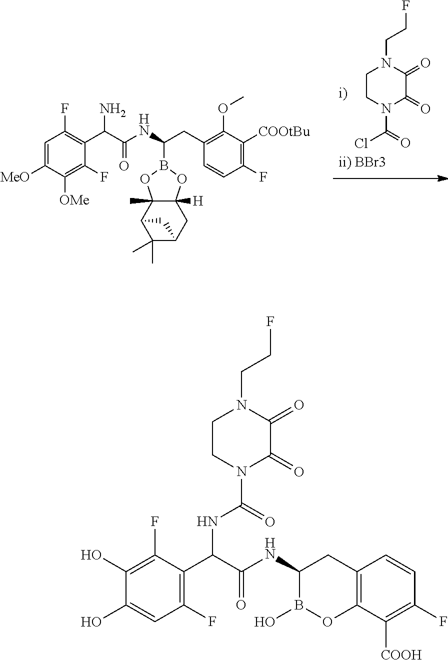

In a similar manner to the synthesis of Example 36, utilizing the above carbonyl chloride in place of 4-ethyl-2,3-dioxopiperazine-1-carbonyl chloride for the acylation in Step 2, the title compound was prepared. ESI-MS m/z 613 (M+1)⁺.

### Example 38: (3R)-3-(2-(4-(2-bromoethyl)-2,3-dioxopiperazine-1-carboxamido)-2-(2,6-difluoro-3,4-dihydroxyphenyl)acetamido)-7-fluoro-2-hydroxy-3,4-dihydro-2H-benzo[e][1,2]oxaborinine-8-carboxylic acid

The title compound was isolated as a byproduct after reversed phase HPLC purification of BBr₃ reaction mixture of Example 37. ESI-MS m/z 674/676 (MH/MH+2)⁺.

### Example 39: (3R)-3-(2-(4-(3-aminopropyl)-2,3-dioxopiperazine-1-carboxamido)-2-(4-carboxyphenyl)acetamido)-2-hydroxy-3,4-dihydro-2H-benzo[e][1,2]oxaborinine-8-carboxylic acid

By following the same procedure of Step 8 of Example 15 utilizing tert-butyl (3-(4-(chlorocarbonyl)-2,3-dioxopiperazin-1-yl)propyl)carbamate (described in Step 1 of Example 27) in place of 4-ethyl-2,3-dioxopiperazine-1-carbonyl chloride, the General Method C using the chloride intermediate (prepared as previous reported, WO 2014/089365), and final deprotection with TMSI as described in Step 9 of Example 15, the title compound was prepared. ESIMS m/z 582 (M+1)+.

### Example 40: (R)-3-((R)-2-(4-(2-aminoethyl)-2,3-dioxopiperazine-1-carboxamido)-2-(2,6-difluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 36, utilizing tert-butyl (2-(4-(chlorocarbonyl)-2,3-dioxopiperazin-1-yl)ethyl)carbamate (described in Step 1 of Example 27) in place of 4-ethyl-2,3-dioxopiperazine-1-carbonyl chloride for the acylation in Step 2, the title compound was prepared after reversed phase HPLC purification, isolated as the first eluting peak. ESI-MS m/z 610 (M+1)⁺.

### Example 41: (R)-3-((R)-2-(4-(3-aminopropyl)-2,3-dioxopiperazine-1-carboxamido)-2-(2,6-difluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 36, utilizing tert-butyl (3-(4-(chlorocarbonyl)-2,3-dioxopiperazin-1-yl)propyl)carbamate (described in Step 1 of Example 27) in place of 4-ethyl-2,3-dioxopiperazine-1-carbonyl chloride for the acylation in Step 2, the title compound was prepared after reversed phase HPLC purification, isolated as the first eluting peak. ESI-MS m/z 624 (M+1)⁺.

### Example 42: (3R)-3-(2-(2-chloro-5-fluoro-3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of 2-chloro-5-fluoro-3,4-dimethoxybenzaldehyde

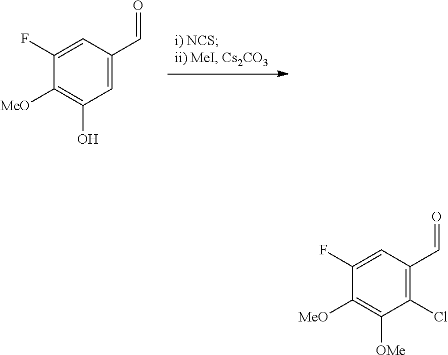 [SMILES: COc1c(O)cc(C=O)cc1F ; COc1c(F)cc(C=O)c(Cl)c1OC]

To 3-Fluoro-4-methoxy-5-hydroxybenzaldehyde (4.6 g, 27.1 mmol) in DMF (50 mL) was added NCS (4.81 g, 36 mmol). The reaction mixture was stirred at RT for 4 h, diluted with diethyl ether, washed with water, brine, dried over Na₂SO₄, concentrated in vacuo to afford the crude product, which was used directly for the next step without further purification. ESI-MS m/z 205/207 (MH/MH+2)⁺. To this crude product in DMF (60 mL) was added Cs₂CO₃ (20.8 g, 63.8 mmol) followed by iodomethane (5 mL, 80 mmol). The reaction mixture was stirred at RT overnight, diluted with diethyl ether, washed with water, brine, dried over Na₂SO₄, concentrated in vacuo. The residue was purified by flash chromatography on silica gel (hexane-EtOAc, 30:1-6:1) to afford the title compound, 1.5 g. ESI-MS m/z 219/221 (MH/MH+2)⁺.

### Step 2. Synthesis of (3R)-3-(2-(2-chloro-5-fluoro-3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

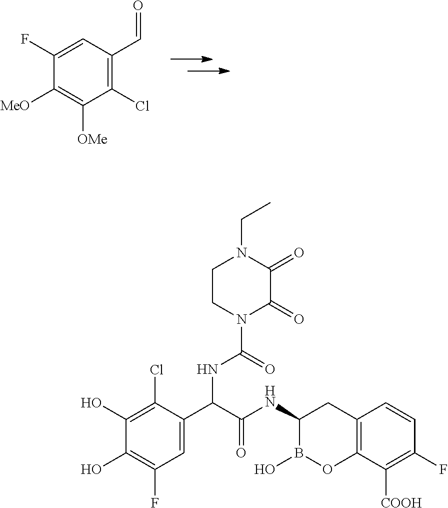 [SMILES: COc1c(F)cc(C=O)c(Cl)c1OC ; CCN1CCN(C(=O)NC(C(=O)N[C@H]2Cc3ccc(F)c(C(=O)O)c3OB2O)c2cc(F)c(O)c(O)c2Cl)C(=O)C1=O]

In a similar manner to the synthesis of Example 36, the title compound was prepared from the above aldehyde. ESI-MS m/z 611/613 (MH/MH+2)⁺.

### Example 43: (3R)-7-fluoro-3-(2-(3-fluoro-5-hydroxypyridin-2-yl)-2-(sulfamoylamino)acetamido)-2-hydroxy-3,4-dihydro-2H-benzo[e][1,2]oxaborinine-8-carboxylic acid

### Step 1: Synthesis of tert-butyl 3-((2R)-2-(2-(5-(benzyloxy)-3-fluoropyridin-2-yl)-2-((N-(tert-butoxycarbonyl)sulfamoyl)amino)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

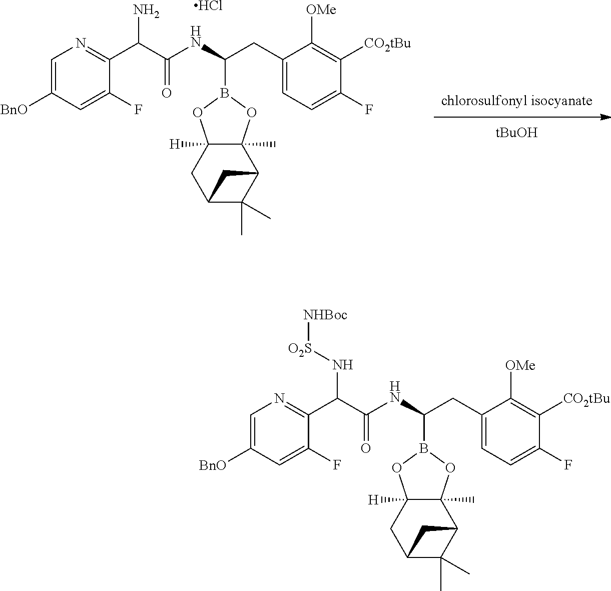 [SMILES: "tBuOH" ; Cl ; COc1c(C[C@H](NC(=O)C(N)c2ncc(OCc3ccccc3)cc2F)B2O[C@@H]3C[C@@H]4C[C@@H](C4(C)C)[C@]3(C)O2)ccc(F)c1C(=O)OC(C)(C)C ; COc1c(C[C@H](NC(=O)C(NS(=O)(=O)NC(=O)OC(C)(C)C)c2ncc(OCc3ccccc3)cc2F)B2O[C@@H]3C[C@@H]4C[C@@H](C4(C)C)[C@]3(C)O2)ccc(F)c1C(=O)OC(C)(C)C]

Solution A—To tert-butyl 3-((2R)-2-(2-amino-2-(5-(benzyloxy)-3-fluoropyridin-2-yl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride 0.25 g (0.34 mmol) in dichloromethane (3.5 mL) at 0° C. was added N,N-diisopropylethylamine 0.065 mL (0.37 mmol, 1.1 eq) and stirred at this temperature. Solution B—To chlorosulfonyl isocyanate 0.032 mL (0.37 mmol, 1.1 eq) in dichloromethane (2 mL) was added a solution of dichloromethane (0.33 mL) and tert-butanol 0.04 mL (0.37 mmol, 1.1 eq) and stirred at RT for 30 minutes. Solution B and N,N-diisopropylethylamine 0.07 mL (0.4 mmol, 1.2 eq) was added to Solution A and warmed at RT for 2 h, The reaction was washed with water/brine, dried over sodium sulfate and concentrated to give the title compound. ESI-MS m/z 885 (MH)⁺.

### Step 2: (3R)-7-fluoro-3-(2-(3-fluoro-5-hydroxypyridin-2-yl)-2-(sulfamoylamino)acetamido)-2-hydroxy-3,4-dihydro-2H-benzo[e][1,2]oxaborinine-8-carboxylic acid

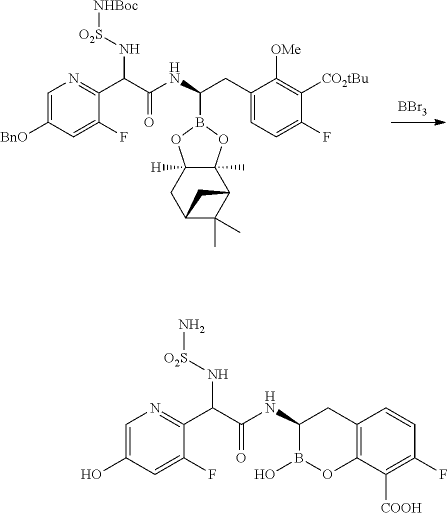 [SMILES: COc1c(C[C@H](NC(=O)C(NS(=O)(=O)NC(=O)OC(C)(C)C)c2ncc(OCc3ccccc3)cc2F)B2O[C@@H]3C[C@@H]4C[C@@H](C4(C)C)[C@]3(C)O2)ccc(F)c1C(=O)OC(C)(C)C ; "BBr3" ; NS(=O)(=O)NC(C(=O)N[C@H]1Cc2ccc(F)c(C(=O)O)c2OB1O)c1ncc(O)cc1F]

The title compound was prepared in a similar manner to the synthesis of Example 1. ESI-MS m/z 473 (MH)⁺.

### Example 44: (3R)-3-(2-(4-carboxy-3-fluorophenyl)-2-(sulfamoylamino)acetamido)-2-hydroxy-3,4-dihydro-2H-benzo[e][1,2]oxaborinine-8-carboxylic acid

The title compound was prepared in a similar manner to the synthesis of Example 43, utilizing 2-(4-(tert-butoxycarbonyl)-3-fluorophenyl)-2-((tert-butoxycarbonyl)amino)acetic acid in place of lithium 2-(5-(benzyloxy)-3-fluoropyridin-2-yl)-2-((tert-butoxycarbonyl)amino)acetate. ESI-MS m/z 482 (MH)⁺.

### Example 45: (3R)-3-(2-(4-carbamoyl-3-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

By following the same procedures of Step 2, 3, 4, 6 of Example 17, 2-((tert-butoxycarbonyl)amino)-2-(4-carbamoyl-3-methoxyphenyl)acetic acid (purchased from Angel Pharmatech, Shanghai, China) was converted to the title compound. ESIMS m/z 568 (M+1)⁺.

### Example 46: (3R)-3-(2-(4-carbamoyl-3-hydroxyphenyl)-2-(4-(2-fluoroethyl)-2,3-dioxopiperazine-1-carboxamido)acetamido)-2-hydroxy-3,4-dihydro-2H-benzo[e][1,2]oxaborinine-8-carboxylic acid

By following the same procedures of Steps 2, 3, 4, 6 of Example 17, utilizing 4-(2-fluoroethyl)-2,3-dioxopiperazine-1-carbonyl chloride (from Step 1 of Example 37) in place of 4-ethyl-2,3-dioxopiperazine-1-carbonyl chloride, 2-((tert-butoxycarbonyl)amino)-2-(4-carbamoyl-3-methoxyphenyl)acetic acid was converted to the title compound. ESIMS m/z 586 (M+1)⁺.

### Example 47: (3R)-3-(2-(4-(3-aminopropyl)-2,3-dioxopiperazine-1-carboxamido)-2-(4-carbamoyl-3-hydroxyphenyl)acetamido)-2-hydroxy-3,4-dihydro-2H-benzo[e][1,2]oxaborinine-8-carboxylic acid

By following the same procedures of Step 2, 3, 4, 6 of Example 17, utilizing tert-butyl (3-(4-(chlorocarbonyl)-2,3-dioxopiperazin-1-yl)propyl)carbamate (described in Step 1 of Example 27) in place of 4-ethyl-2,3-dioxopiperazine-1-carbonyl chloride, 2-((tert-butoxycarbonyl)amino)-2-(4-carbamoyl-3-methoxyphenyl)acetic acid was converted to the title compound. ESIMS m/z 597 (M+1)⁺.

### Example 48: (3R)-3-(2-(4-carboxy-3-methoxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

By following the same procedure of Step 8 of Example 15, the General Method C using the chloride intermediate (prepared as previous reported, WO 2014/089365), and final deprotection with TMSI as described in Step 9 of Example 15, 2-amino-2-(4-(tert-butoxycarbonyl)-3-methoxyphenyl)acetic acid (purchased from Angel Pharmatech, Shanghai, China) was converted to the title compound. ESIMS m/z 583 (M+1)⁺.

### Example 49: (3R)-7-fluoro-3-(2-(3-fluoro-5-hydroxypyridin-2-yl)-2-(3-phenylureido)acetamido)-2-hydroxy-3,4-dihydro-2H-benzo[e][1,2]oxaborinine-8-carboxylic acid

The title compound was prepared in a similar manner to the synthesis of Example 1, utilizing phenylcarbamic chloride in place of tert-butyl (3-(4-(chlorocarbonyl)-2,3-dioxopiperazin-1-yl)propyl)carbamate. ESI-MS m/z 513 (MH)⁺.

### Example 50: (3R)-3-(2-(4-amino-3-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1: Synthesis of benzyl (2-fluoro-4-formylphenyl)carbamate

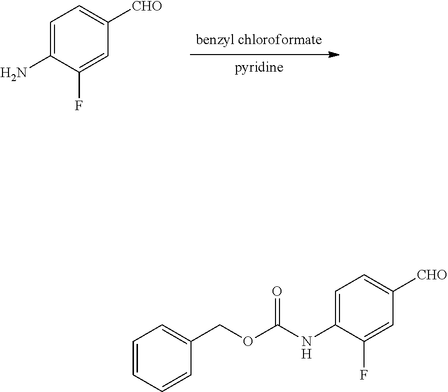 [SMILES: Nc1ccc(C=O)cc1F ; "benzyl chloroformate" ; "pyridine" ; O=Cc1ccc(NC(=O)OCc2ccccc2)c(F)c1]

To 4-amino-3-fluorobenzaldehyde 1 g (7.19 mmol) in dichloromethane (10 mL) at 0° C. was added pyridine 1.75 mL (21.6 mmol, 3 eq), followed by benzyl chloroformate 1.52 mL (10.8 mmol, 1.5 eq) and the reaction was warmed at RT for 18 h. The product was washed with water/brine, dried over sodium sulfate, concentrated and purified by flash chromatography on silica gel (30% ethyl acetate/hexanes) to give the title compound, 0.98 g, (50%). ESI-MS m/z 274 (MH)⁺.

### Step 2: Synthesis of (3R)-3-(2-(4-amino-3-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

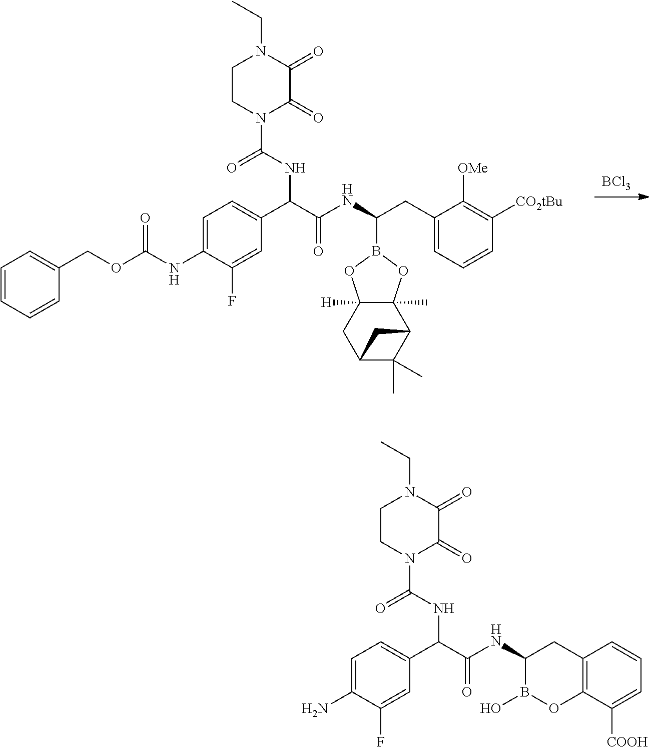 [SMILES: CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ccc(NC(=O)OCc3ccccc3)c(F)c2)C(=O)C1=O ; "BCl3" ; CCN1CCN(C(=O)NC(C(=O)N[C@H]2Cc3cccc(C(=O)O)c3OB2O)c2ccc(N)c(F)c2)C(=O)C1=O]

The title compound was prepared in a similar manner to the synthesis of Example 1, utilizing 2-(4-(((benzyloxy)carbonyl)amino)-3-fluorophenyl)-2-((tert-butoxycarbonyl)amino)acetic acid in place of lithium 2-(5-(benzyloxy)-3-fluoropyridin-2-yl)-2-((tert-butoxycarbonyl)amino)acetate. ESI-MS m/z 542 (MH)⁺.

### Example 51: (3R)-7-fluoro-3-(2-(3-fluoro-5-hydroxypyridin-2-yl)-2-(4-(2-guanidinoethyl)-2,3-dioxopiperazine-1-carboxamido)acetamido)-2-hydroxy-3,4-dihydro-2H-benzo[e][1,2]oxaborinine-8-carboxylic acid

### Step 1: Synthesis of tert-butyl 3-((2R)-2-(2-(5-(benzyloxy)-3-fluoropyridin-2-yl)-2-(4-(2-((Z)-2,3-bis(tert-butoxycarbonyl)guanidino)ethyl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

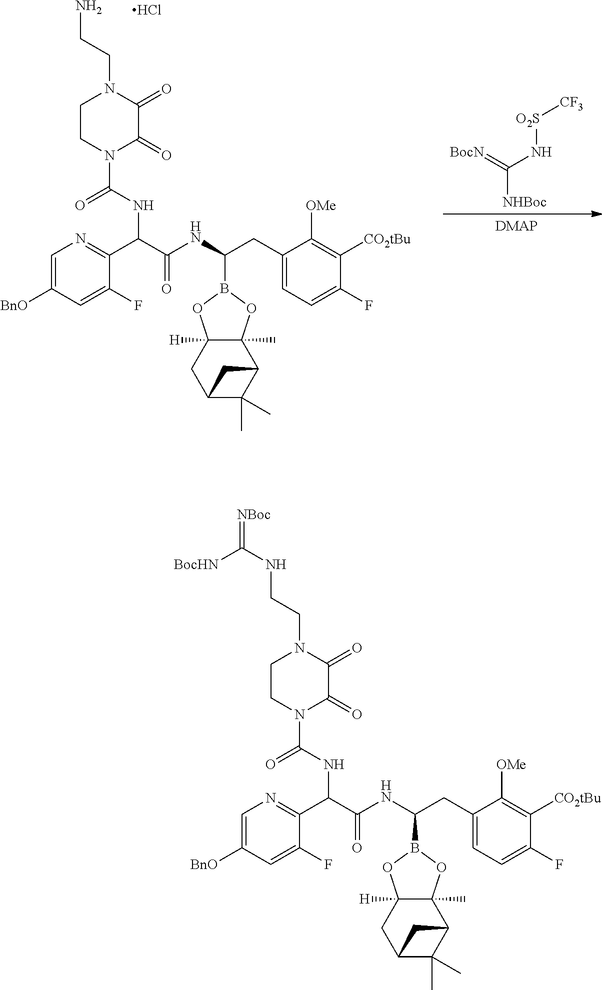

To tert-butyl 3-((2R)-2-(2-(4-(2-aminoethyl)-2,3-dioxopiperazine-1-carboxamido)-2-(5-(benzyloxy)-3-fluoropyridin-2-yl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride 0.322 g (0.35 mmol) in dichloromethane (8 mL) was added N,N-diisopropylethylamine 0.18 mL (1.05 mmol, 3 eq), 1,3-di-boc-2-(trifluoromethyl)guanidine 0.09 g (0.23 mmol, 0.7 eq), followed by 4-(dimethylamino) pyridine 0.002 g (0.02 mmol, 0.05 eq) and the reaction was stirred at RT for 18 h. The product was washed with water/brine, dried over sodium sulfate and concentrated to give the title compound. ESI-MS m/z 1131 (MH)⁺.

### Step 2: Synthesis of (3R)-7-fluoro-3-(2-(3-fluoro-5-hydroxypyridin-2-yl)-2-(4-(2-guanidinoethyl)-2,3-dioxopiperazine-1-carboxamido)acetamido)-2-hydroxy-3,4-dihydro-2H-benzo[e][1,2]oxaborinine-8-carboxylic acid

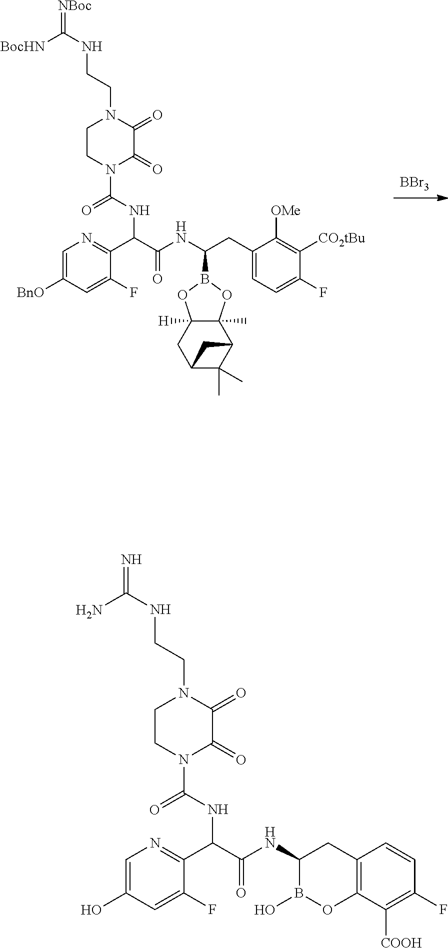

The title compound was prepared in a similar manner to the synthesis of Example 1. ESI-MS m/z 619 (MH)⁺.

### Example 52: (3R)-7-fluoro-3-(2-(3-fluoro-5-hydroxypyridin-2-yl)-2-guanidinoacetamido)-2-hydroxy-3,4-dihydro-2H-benzo[e][1,2]oxaborinine-8-carboxylic acid

### Step 1: Synthesis of tert-butyl 3-((11R,E)-8-(5-(benzyloxy)-3-fluoropyridin-2-yl)-6-((tert-butoxycarbonyl)amino)-2,2-dimethyl-4,9-dioxo-11-((3aS,4S,6S,7aR)-3a,5,5-trimethylhexahydro-4,6-methanobenzo[d][1,3,2]dioxaborol-2-yl)-3-oxa-5,7,10-triazadodec-5-en-12-yl)-6-fluoro-2-methoxybenzoate

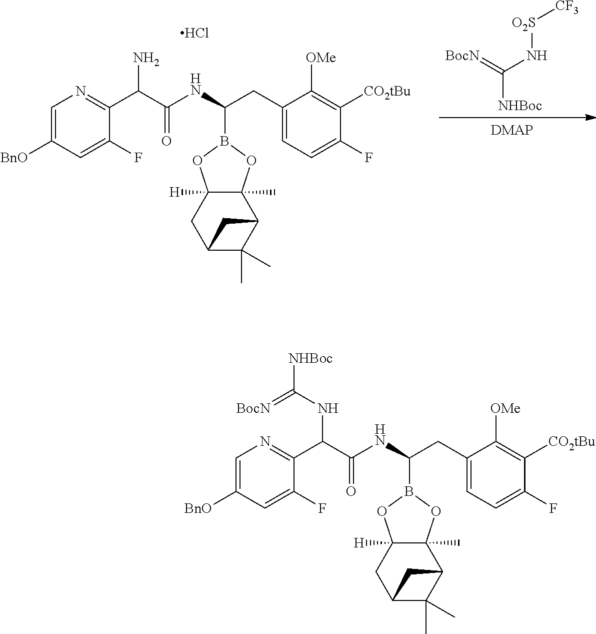

To tert-butyl 3-((2R)-2-(2-amino-2-(5-(benzyloxy)-3-fluoropyridin-2-yl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride 0.12 g (0.16 mmol) in dichloromethane (3 mL) at 0° C. was added N,N-diisopropylethylamine 0.09 mL (0.49 mmol, 3 eq), 1,3-di-boc-2-(trifluoromethyl)guanidine 0.0.033 g (0.11 mmol, 0.7 eq), followed by 4-(dimethylamino) pyridine 0.01 g (0.08 mmol, 0.5 eq) and the reaction was stirred at RT for 18 h. The product was washed with 1N aqueous hydrogen chloride/water/brine, dried over sodium sulfate and concentrated to give the title compound. ESI-MS m/z 948 (MH)⁺.

### Step 2: Synthesis of (3R)-7-fluoro-3-(2-(3-fluoro-5-hydroxypyridin-2-yl)-2-guanidinoacetamido)-2-hydroxy-3,4-dihydro-2H-benzo[e][1,2]oxaborinine-8-carboxylic acid

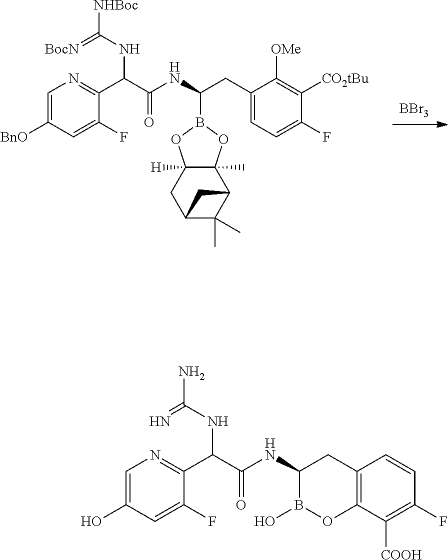

The title compound was prepared in a similar manner to the synthesis of Example 1. ESI-MS m/z 436 (MH)⁺.

### Example 53: (3R)-7-fluoro-3-(2-(3-fluoro-5-hydroxypyridin-2-yl)-2-(2-fluoroisonicotinamido)acetamido)-2-hydroxy-3,4-dihydro-2H-benzo[e][1,2]oxaborinine-8-carboxylic acid

### Step 1: Synthesis of tert-butyl 3-((2R)-2-(2-(5-(benzyloxy)-3-fluoropyridin-2-yl)-2-(2-fluoroisonicotinamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

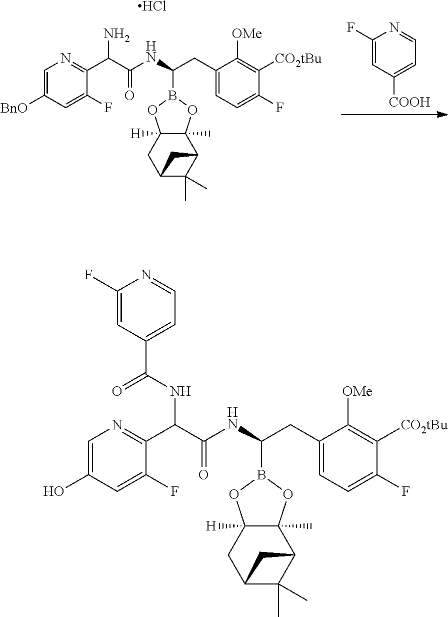 [SMILES: Cl ; COc1c(C[C@H](NC(=O)C(N)c2ncc(OCc3ccccc3)cc2F)B2O[C@@H]3C[C@@H]4C[C@@H](C4(C)C)[C@]3(C)O2)ccc(F)c1C(=O)OC(C)(C)C ; O=C(O)c1ccnc(F)c1 ; COc1c(C[C@H](NC(=O)C(NC(=O)c2ccnc(F)c2)c2ncc(O)cc2F)B2O[C@@H]3C[C@@H]4C[C@@H](C4(C)C)[C@]3(C)O2)ccc(F)c1C(=O)OC(C)(C)C]

To tert-butyl 3-((2R)-2-(2-amino-2-(5-(benzyloxy)-3-fluoropyridin-2-yl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride 0.15 g (0.2 mmol) in dichloromethane (2 mL) was added triethylamine 0.09 mL (0.61 mmol, 3 eq), N-(3-dimethylaminopropyl)-N′-ethylcarbodiimide hydrochloride 0.043 g (0.22 mmol, 1.1 eq), 1-hydroxybenzotriazole hydrate 0.03 g (0.22 mmol, 1.1 eq), followed by 2-fluoroisonicotinic acid 0.034 g (0.24 mmol, 1.2 eq) and the reaction was stirred at RT for 18 h. The product was quenched with water, washed with brine, dried over sodium sulfate and concentrated to give the title compound. ESI-MS m/z 829 (MH)⁺.

### Step 2: Synthesis of (3R)-7-fluoro-3-(2-(3-fluoro-5-hydroxypyridin-2-yl)-2-(2-fluoroisonicotinamido)acetamido)-2-hydroxy-3,4-dihydro-2H-benzo[e][1,2]oxaborinine-8-carboxylic acid

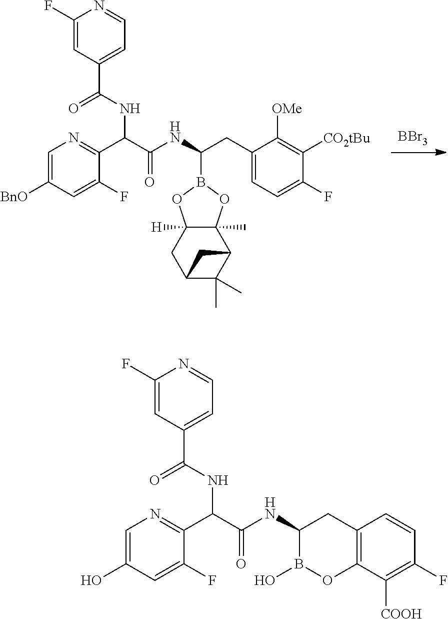 [SMILES: COc1c(C[C@H](NC(=O)C(NC(=O)c2ccnc(F)c2)c2ncc(OCc3ccccc3)cc2F)B2O[C@@H]3C[C@@H]4C[C@@H](C4(C)C)[C@]3(C)O2)ccc(F)c1C(=O)OC(C)(C)C ; "BBr3" ; O=C(NC(C(=O)N[C@H]1Cc2ccc(F)c(C(=O)O)c2OB1O)c1ncc(O)cc1F)c1ccnc(F)c1]

The title compound was prepared in a similar manner to the synthesis of Example 1. ESI-MS m/z 517 (MH)⁺.

### Example 54: (3R)-3-(2-(2-aminothiazol-4-yl)-2-(4-ethyl-2,3-dioxopiperazine-1-carboxamido)acetamido)-2-hydroxy-7-mercapto-3,4-dihydro-2H-benzo[e][1,2]oxaborinine-8-carboxylic acid

### Step 1. Synthesis of tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-((4-methoxybenzyl)thio)benzoate

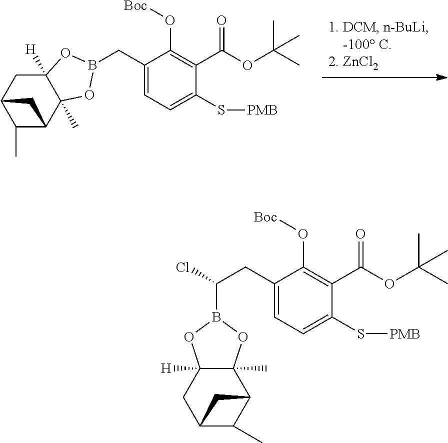

To a solution of THF (100 mL) and DCM (9 mL, 140 mmol) at −100° C. under argon was added n-BuLi (2.5 M in hexane, 29 mL, 72.5 mmol) dropwise over 45 min. After the resulting mixture was stirred for an additional hour at −100° C., a solution of tert-butyl 2-((tert-butoxycarbonyl)oxy)-6-((4-methoxybenzyl)thio)-3-(((3aS,4S,6S,7aR)-3a,5,5-trimethylhexahydro-4,6-methanobenzo[d][1,3,2]dioxaborol-2-yl)methyl)benzoate (26.41 g, 41.35 mmol) in THF (80 mL) was added dropwise over 40 min. The reaction mixture was stirred for an additional hour at −100° C. and then ZnCl₂ (1.0 M in diethyl ether, 73 mL, 73 mmol) was added dropwise over 30 min. The mixture was allowed to slowly warm to −50° C. over 2 h and then at −10° C. for 1.5 h. The reaction mixture was then quenched with saturated NH₄Cl (120 mL) and extracted with ethyl acetate (3×). The combined organic phase was dried over Na₂SO₄. After the solvent was evaporated under reduced pressure, the residue was purified by flash chromatography on silica gel (340 g column) eluted with 0 to 20% ethyl acetate/hexanes to afford 23.48 g (82.6%) of tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-((4-methoxybenzyl)thio)benzoate. ESI-MS m/z 709.2 (M+Na)⁺.

### Step 2. Synthesis of tert-butyl 3-((2R)-2-(2-(2-(((benzyloxy)carbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((4-methoxybenzyl)thio)benzoate

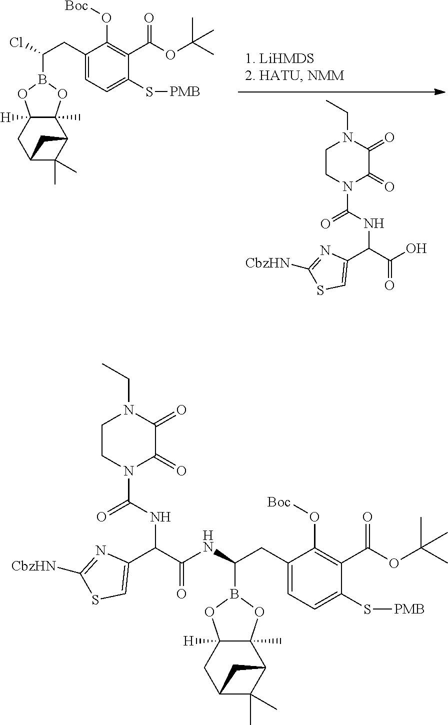

To a solution of tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-((4-methoxybenzyl)thio)benzoate (11.70 g, 17.0 mmol) in THF (125 mL) at −78° C. under argon was added LiHMDS (1.0 M in THF, 14 mL, 14 mmol) dropwise. The resulting mixture was allowed to slowly warm to room temperature overnight.

A mixture of 2-(2-(((benzyloxy)carbonyl)amino)thiazol-4-yl)-2-(4-ethyl-2,3-dioxopiperazine-1-carboxamido)acetic acid (7.24 g, 15.2 mmol), HATU (7.70 g, 20.2 mmol), NMM (2.8 mL, 25.5 mmol) in DMA (100 mL) was stirred at room temperature under argon for 1 h and then added to the reaction mixture above via cannula.

The resulting reaction mixture was stirred at room temperature for 26 h and then quenched with water, extracted with ethyl acetate (3×). The combined organic phase was dried over Na₂SO₄. After the solvent was evaporated under reduced pressure, the residue was purified by flash chromatography on silica gel (340 g column) eluted with 0 to 500% acetone/hexanes to afford 8.32 g (52.80%) of tert-butyl 3-((2R)-2-(2-(2-(((benzyloxy)carbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((4-methoxybenzyl)thio)benzoate as a solid. ESI-MS m/z 1125.4 (M+H)⁺.

### Step 3. Synthesis of (3R)-3-(2-(2-aminothiazol-4-yl)-2-(4-ethyl-2,3-dioxopiperazine-1-carboxamido)acetamido)-2-hydroxy-7-mercapto-3,4-dihydro-2H-benzo[e][1,2]oxaborinine-8-carboxylic acid

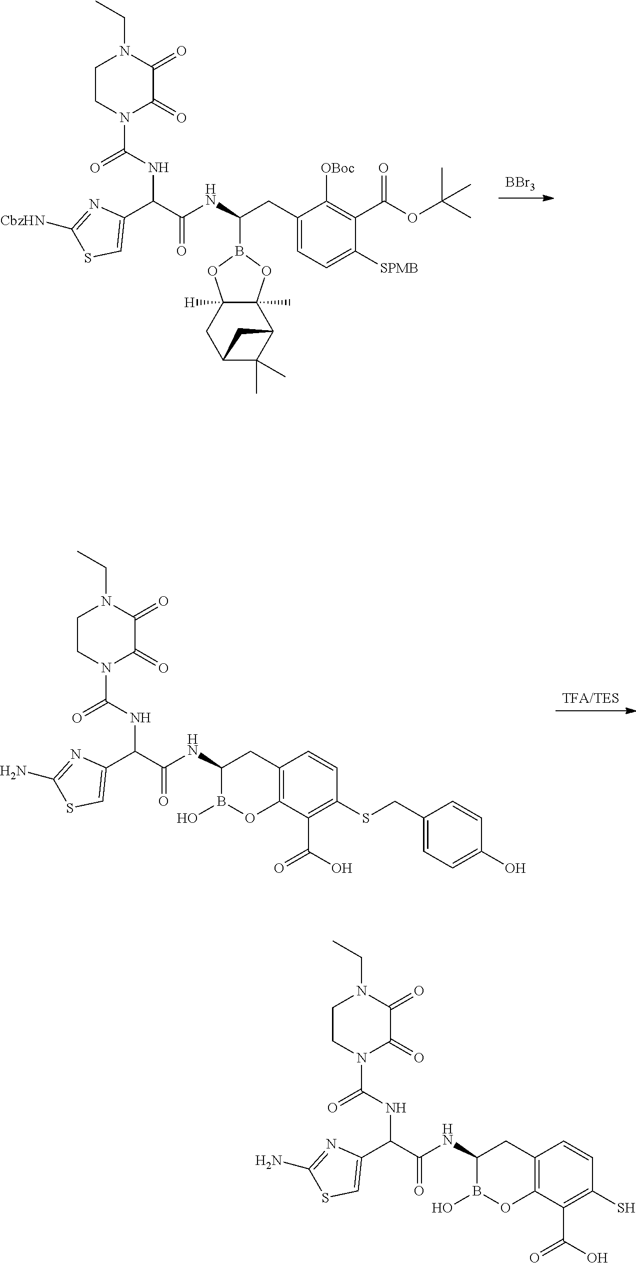

To a solution of tert-butyl 3-((2R)-2-(2-(2-(((benzyloxy)carbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((4-methoxybenzyl)thio)benzoate (0.17 g, 0.15 mmol) in DCM (12 mL) was added BBr₃ (1.0 M in DCM, 2.4 mL, 2.4 mmol) dropwise at −78° C. under argon. The mixture was allowed to slowly warm to −15° C. over 2 h and then stirred at 0° C. for 3 h. The reaction mixture was then quenched with water (1 mL) and MeOH (10 mL), evaporated under reduced pressure. The residue was filtered and washed with water and t-BuOMe, and dried under high vacuum to afford the crude (3R)-3-(2-(2-aminothiazol-4-yl)-2-(4-ethyl-2,3-dioxopiperazine-1-carboxamido)acetamido)-2-hydroxy-7-((4-hydroxybenzyl)thio)-3,4-dihydro-2H-benzo[e][1,2]oxaborinine-8-carboxylic acid. ESI-MS m/z 690.9 (M+Na)⁺, 85.7 mg of which was treated with TFA (5 mL) and triethylsilane (1 mL) at room temperature for 15 h. The reaction mixture was evaporated under reduced pressure, the residue was filtered and washed with MeOH yielding (3R)-3-(2-(2-aminothiazol-4-yl)-2-(4-ethyl-2,3-dioxopiperazine-1-carboxamido)acetamido)-2-hydroxy-7-mercapto-3,4-dihydro-2H-benzo[e][1,2]oxaborinine-8-carboxylic acid. ESI-MS m/z 562.2 (M+H)⁺.

### Example 55: (3R)-3-(2-(2-aminothiazol-4-yl)-2-(4-ethyl-2,3-dioxopiperazine-1-carboxamido)acetamido)-2-hydroxy-7-((4-hydroxybenzyl)thio)-3,4-dihydro-2H-benzo[e][1,2]oxaborinine-8-carboxylic acid

The title compound was prepared as described above (Example 54, step 3) and purified by reverse phase HPLC (Gilson, C18 column) and then lyophilized to give (3R)-3-(2-(2-aminothiazol-4-yl)-2-(4-ethyl-2,3-dioxopiperazine-1-carboxamido)acetamido)-2-hydroxy-7-((4-hydroxybenzyl)thio)-3,4-dihydro-2H-benzo[e][1,2]oxaborinine-8-carboxylic acid as TFA salt. ESI-MS m/z 690.9 (M+Na)⁺.

### Example 56: (3R)-3-(2-(2-aminothiazol-4-yl)-2-(4-ethyl-2,3-dioxopiperazine-1-carboxamido)acetamido)-2-hydroxy-7-(methylthio)-3,4-dihydro-2H-benzo[e][1,2]oxaborinine-8-carboxylic acid

### Step 1. Synthesis of tert-butyl 3-bromo-2-((tert-butoxycarbonyl)oxy)-6-fluorobenzoate

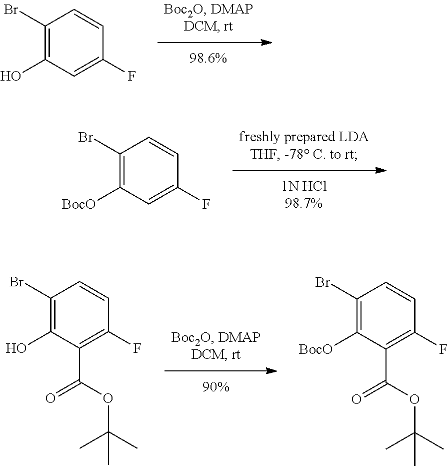

To a mixture of 2-bromo-5-fluorophenol (50.70 g, 265 mmol) and Boc₂O (70.05 g, 321 mmol) in DCM (300 mL) was added DMAP (1.66 g, 13.6 mmol) at room temperature. The reaction mixture was stirred at room temperature for 2 h. After the solvent was evaporated under reduced pressure, the residue was purified by flash chromatography on silica gel (340 g column) eluted with 0 to 20% ethyl acetate/hexanes to afford 76.24 g (98.6%) of 2-bromo-5-fluorophenyl tert-butyl carbonate. ESI-MS m/z 275.9, 277.9 (M-15)⁺.

To a solution of 2-bromo-5-fluorophenyl tert-butyl carbonate (76.23 g, 262 mmol) in THF (450 mL) at −78° C. under argon was added freshly prepared LDA, n-BuLi (2.5 M in hexane, 126 mL, 315 mmol) was added dropwise to a solution of DIPA (44.5 mL, 317 mmol) in THF (250 mL) at −78° C. under argon and the resulting solution was stirred at −78° C. for 1.5 h, via cannula. The reaction mixture was allowed to slowly warm to room temperature overnight and then quenched with 2 N HCl (350 mL), extracted with ethyl acetate (3×), dried over Na₂SO₄, and evaporated under reduced pressure to afford 75.27 g (98.7%) of tert-butyl 3-bromo-6-fluoro-2-hydroxybenzoate as a solid. ESI-MS m/z 275.9, 277.9 (M-15)⁺, 234.9, 236.9 (M-56)⁺.

To a mixture of tert-butyl 3-bromo-6-fluoro-2-hydroxybenzoate (20.64 g, 70.9 mmol) and Boc₂O (24.18 g, 110 mmol) in DCM (200 mL) was added DMAP (0.526 g, 4.3 mmol) at room temperature. The reaction mixture was stirred at room temperature for 20 h. After the solvent was evaporated under reduced pressure, the residue was purified by flash chromatography on silica gel (340 g column) eluted with 0 to 20% ethyl acetate/hexanes to afford 25.02 g (90%) of tert-butyl 3-bromo-2-((tert-butoxycarbonyl)oxy)-6-fluorobenzoate as a solid. ESI-MS m/z 805.0, 807.0 (2M+Na)⁺.

### Step 2. Synthesis of tert-butyl 3-bromo-2-((tert-butoxycarbonyl)oxy)-6-(methylthio)benzoate

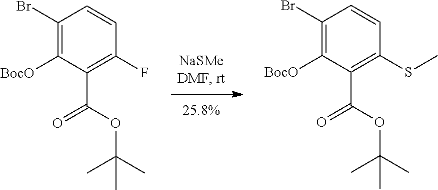

A mixture of tert-butyl 3-bromo-2-((tert-butoxycarbonyl)oxy)-6-fluorobenzoate (22.21 g, 56.8 mmol) and NaSMe (6.12 g, 87.3 mmol) in DMF (100 mL) was stirred at room temperature for 4 d. The reaction mixture was then quenched with water, extracted with ethyl acetate (3×). The combined organic phase was dried over Na₂SO₄. After the solvent was evaporated under reduced pressure, the residue was purified by flash chromatography on silica gel (340 g column) eluted with 0 to 20% ethyl acetate/hexanes to afford 6.14 g (25.8%) of tert-butyl 3-bromo-2-((tert-butoxycarbonyl)oxy)-6-(methylthio)benzoate as a solid. ESI-MS m/z 861.0, 863.0 (2M+Na)⁺.

### Step 3. Synthesis of tert-butyl 2-((tert-butoxycarbonyl)oxy)-6-(methylthio)-3-(((3aS,4S,6S,7aR)-3a,5,5-trimethylhexahydro-4,6-methanobenzo[d][1,3,2]dioxaborol-2-yl)methyl)benzoate

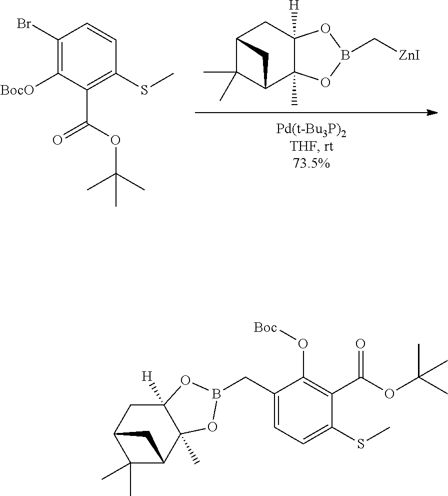

To a solution of tert-butyl 3-bromo-2-((tert-butoxycarbonyl)oxy)-6-(methylthio)benzoate (2.58 g, 6.15 mmol) and bis(tri-tert-butylphosphine)palladium(0) (0.35 g, 0.68 mmol) in THF (20 mL) at room temperature under argon was added a solution of (((3aS,4S,6S,7aR)-3a,5,5-trimethylhexahydro-4,6-methanobenzo[d][1,3,2]dioxaborol-2-yl)methyl)zinc(II) iodide (0.5 M in THF, 40 mL, 20 mmol), prepared as described in Example 59 step 3. The resulting mixture was stirred at room temperature for 28 h. After the solvent was evaporated under reduced pressure, the residue was purified by flash chromatography on silica gel (340 g column) eluted with 0 to 20% ethyl acetate/hexanes to afford 2.41 g (73.5%) of tert-butyl 2-((tert-butoxycarbonyl)oxy)-6-(methylthio)-3-(((3aS,4S,6S,7aR)-3a,5,5-trimethylhexahydro-4,6-methanobenzo[d][1,3,2]dioxaborol-2-yl)methyl)benzoate. ESI-MS m/z 1087.4 (2M+Na)⁺.

### Step 4. Synthesis of tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-(methylthio)benzoate

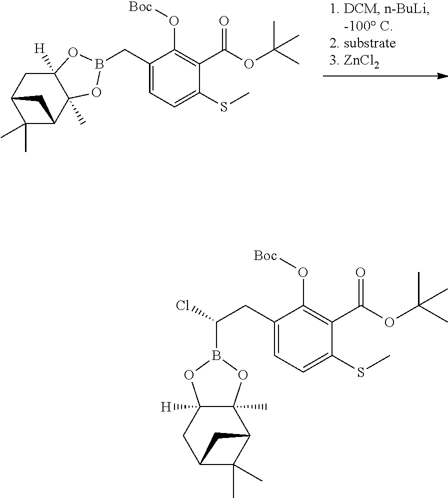 [SMILES: CSc1ccc(CB2O[C@@H]3C[C@@H]4C[C@@H](C4(C)C)[C@]3(C)O2)c(OC(=O)OC(C)(C)C)c1C(=O)OC(C)(C)C ; CSc1ccc(C[C@@H](Cl)B2O[C@@H]3C[C@@H]4C[C@@H](C4(C)C)[C@]3(C)O2)c(OC(=O)OC(C)(C)C)c1C(=O)OC(C)(C)C]

The Matteson reaction was carried out as described in Example 54 step 1 by using tert-butyl 2-((tert-butoxycarbonyl)oxy)-6-(methylthio)-3-(((3aS,4S,6S,7aR)-3a,5,5-trimethylhexahydro-4,6-methanobenzo[d][1,3,2]dioxaborol-2-yl)methyl)benzoate as the substrate to afford tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-(methylthio)benzoate. ESI-MS m/z 1183.4 (2M+Na)⁺.

### Step 5. Synthesis of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-(methylthio)benzoate

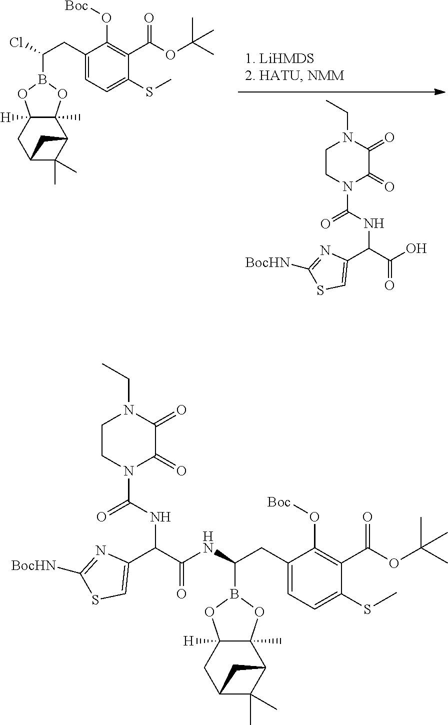 [SMILES: CSc1ccc(C[C@@H](Cl)B2O[C@@H]3C[C@@H]4C[C@@H](C4(C)C)[C@]3(C)O2)c(OC(=O)OC(C)(C)C)c1C(=O)OC(C)(C)C ; CCN1CCN(C(=O)NC(C(=O)O)c2csc(NC(=O)OC(C)(C)C)n2)C(=O)C1=O ; CCN1CCN(C(=O)NC(C(=O)N[C@@H](Cc2ccc(SC)c(C(=O)OC(C)(C)C)c2OC(=O)OC(C)(C)C)B2O[C@@H]3C[C@@H]4C[C@@H](C4(C)C)[C@]3(C)O2)c2csc(NC(=O)OC(C)(C)C)n2)C(=O)C1=O]

The amide formation was carried out as described in Example 59 step 7 by using tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-(methylthio)benzoate as the substrate to afford tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-(methylthio)benzoate. ESI-MS m/z 985.4 (M+H)⁺.

### Step 6. Synthesis of (3R)-3-(2-(2-aminothiazol-4-yl)-2-(4-ethyl-2,3-dioxopiperazine-1-carboxamido)acetamido)-2-hydroxy-7-(methylthio)-3,4-dihydro-2H-benzo[e][1,2]oxaborinine-8-carboxylic acid

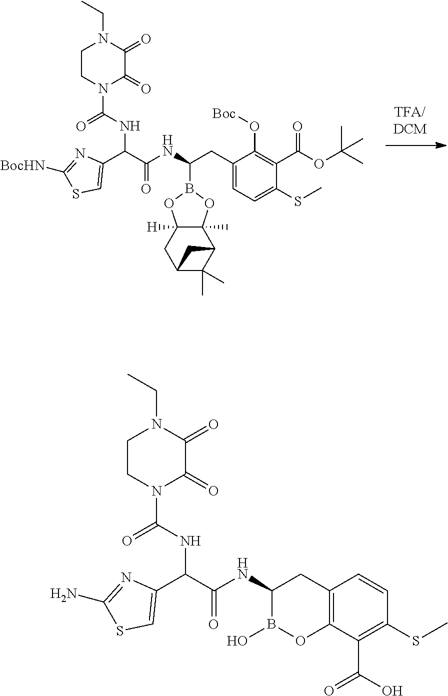 [SMILES: CCN1CCN(C(=O)NC(C(=O)N[C@@H](Cc2ccc(SC)c(C(=O)OC(C)(C)C)c2OC(=O)OC(C)(C)C)B2O[C@@H]3C[C@@H]4C[C@@H](C4(C)C)[C@]3(C)O2)c2csc(NC(=O)OC(C)(C)C)n2)C(=O)C1=O ; CCN1CCN(C(=O)NC(C(=O)N[C@H]2Cc3ccc(SC)c(C(=O)O)c3OB2O)c2csc(N)n2)C(=O)C1=O]

The full deprotection of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-(methylthio)benzoate was carried out as described in General Method A with TFA (20% in DCM) at 0° C. for 8 h, purified by reverse phase HPLC (Gilson, C18 column) and then lyophilized to give (3R)-3-(2-(2-aminothiazol-4-yl)-2-(4-ethyl-2,3-dioxopiperazine-1-carboxamido)acetamido)-2-hydroxy-7-(methylthio)-3,4-dihydro-2H-benzo[e][1,2]oxaborinine-8-carboxylic acid as TFA salt. ESI-MS m/z 577.1 (M+H)⁺.

### Example 57: (3R)-3-(2-(2-aminothiazol-4-yl)-2-(4-ethyl-2,3-dioxopiperazine-1-carboxamido)acetamido)-7-formyl-2-hydroxy-3,4-dihydro-2H-benzo[e][1,2]oxaborinine-8-carboxylic acid

### Step 1. Synthesis of ethyl 2-(2-aminothiazol-4-yl)-2-((tert-butoxycarbonyl)amino)acetate

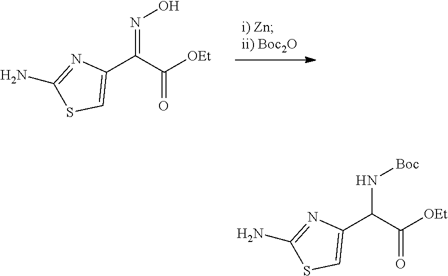

To ethyl 2-amino-α-(hydroxyimino)-4-thiazoleacetate (4.4 g, 20 mmol) in 50% HCOOH (40 mL) and MeOH (20 mL) was added zinc dust (3 g, 46 mmol) at 0° C. The reaction mixture was stirred 0° C. for 3 h, filtered through a pad of Celite, the filtrate was concentrated. To this concentrated mixture was added water (80 mL), basified with K₂CO₃ to pH ˜8-9, then THF (100 mL) was added to the resulting solution followed by Boc₂O (5.24 g, 24 mmol). The reaction was stirred at 0° C. for 1 h, then warmed to RT, added more Boc₂O (1.9 g, 8.7 mmol), stirred for an additional 1 h 40 min, extracted with EtOAc. The organic extracts were dried over Na₂SO₄, and concentrated. The crude product was purified by flash chromatography on silica gel (hexane-EtOAc, 10:1-1:3) to afford the title compound, 4.5 g. ESI-MS m/z 302 (M+1)⁺.

### Step 2. Synthesis of ethyl 2-(2-(((benzyloxy)carbonyl)amino)thiazol-4-yl)-2-((tert-butoxycarbonyl)amino)acetate

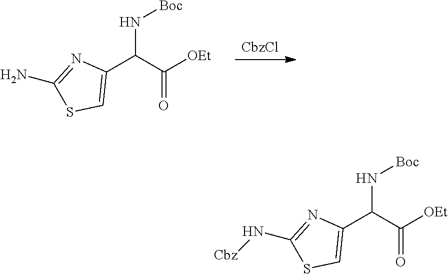 [SMILES: CCOC(=O)C(NC(=O)OC(C)(C)C)c1csc(N)n1 ; "CbzCl" ; CCOC(=O)C(NC(=O)OC(C)(C)C)c1csc(NC(=O)OCc2ccccc2)n1]

By following standard procedures for CBZ protection of an amine, the title compound was prepared from the above product. ESI-MS m/z 436 (M+1)⁺.

### Step 3. Synthesis of 2-(2-(((benzyloxy)carbonyl)amino)thiazol-4-yl)-2-((tert-butoxycarbonyl)amino)acetic acid

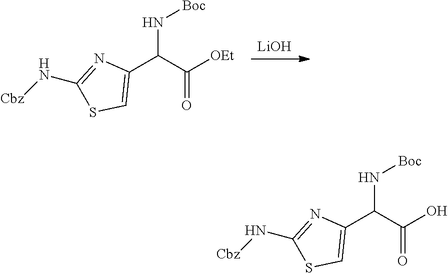

The above product (6.53 g, 15 mmol) in THF (100 mL) and water (100 mL) was treated with lithium hydroxide monohydrate (1.89 g, 45 mmol) at RT for 2 h, then concentrated in vacuo, acidified with 1 N HCl to pH ˜ 3-4. The precipitated solid was collected by filtration, washed with water, and dried in vacuo to afford the title compound, 5.8 g. ESI-MS m/z 408 (M+1)⁺.

### Step 4. Synthesis of 2-amino-2-(2-(((benzyloxy)carbonyl)amino)thiazol-4-yl)acetic acid

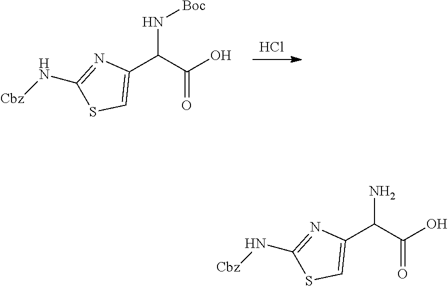 [SMILES: CC(C)(C)OC(=O)NC(C(=O)O)c1csc(NC(=O)OCc2ccccc2)n1 ; Cl ; NC(C(=O)O)c1csc(NC(=O)OCc2ccccc2)n1]

To a solution of the above product (5.8 g, 14.25 mmol) in DCM (120 mL) was added 4.0 M HCl in dioxane solution (60 mL, 240 mmol). The reaction mixture was stirred at RT overnight, diluted with diethyl ether. The precipitated solid was collected by filtration, washed with diethyl ether, and dried in vacuo to afford the title compound as the HCl salt, 4.86 g. ESI-MS m/z 308 (M+1)⁺.

### Step 5. Synthesis of 2-(2-(((benzyloxy)carbonyl)amino)thiazol-4-yl)-2-(4-ethyl-2,3-dioxopiperazine-1-carboxamido)acetic acid

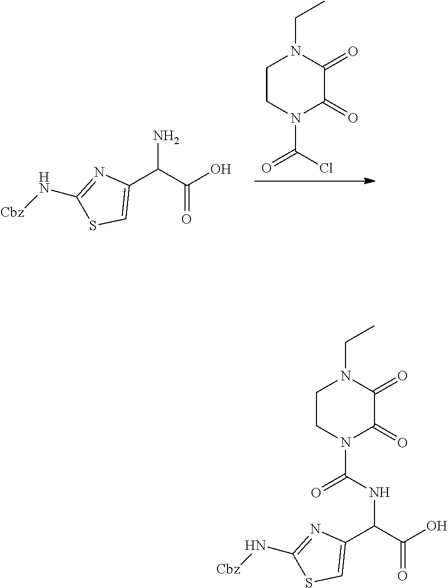 [SMILES: NC(C(=O)O)c1csc(NC(=O)OCc2ccccc2)n1 ; CCN1CCN(C(=O)Cl)C(=O)C1=O ; CCN1CCN(C(=O)NC(C(=O)O)c2csc(NC(=O)OCc3ccccc3)n2)C(=O)C1=O]

To a solution of the above amino acid (2.06 g, 6 mmol) in THF (60 mL) and water (60 mL) was added a solution of NaOH (480 mg, 12 mmol) in water (5 mL) at 0° C., followed by aqueous saturated NaHCO₃ (20 mL), a solution of 4-ethyl-2,3-dioxopiperazine-1-carbonyl chloride (1.54 g, 7.5 mmol) in THF (8 mL), and MeOH (160 mL). The reaction mixture was stirred between 0-10° C. for 1.5 h, then concentrated in vacuo, acidified with 1 N HCl to pH ˜ 2, extracted with EtOAc. The organic extracts were combined, dried over Na₂SO₄, concentrated in vacuo to afford the crude product, 2.3 g, which was used directly for the next Step without further purification. ESI-MS m/z 476 (M+1)⁺.

### Step 6. Synthesis of tert-butyl 3-((tert-butyldimethylsilyl)oxy)-6-(1,3-dioxan-2-yl)-2-methoxybenzoate

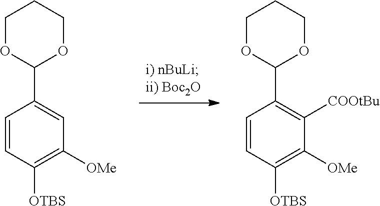

To a solution of (4-(1,3-dioxan-2-yl)-2-methoxyphenoxy)(tert-butyl)dimethylsilane (15 g, 46.3 mmol) (which was prepared according to the reported procedures, Synlett, 2004, 2736-2738) in anhydrous cyclohexane (240 mL) was added nBuLi (2.5 M, 27 mL, 67.5 mmol) dropwise at 0° C. under argon. The reaction mixture was stirred at RT for 3 h, then re-cooled to 0° C., Boc₂O (36.7 g, 168 mmol) was added. The reaction mixture was stirred at RT overnight, quenched with aqueous Na₂CO₃, extracted with diethyl ether. The ether extracts were washed with brine, dried over Na₂SO₄, and concentrated. The crude product was purified by flash chromatography on silica gel (hexane-EtOAc, 60:1-6:1) to afford the title compound, 13 g. ESI-MS m/z 871 (2M+Na)⁺.

### Step 7. Synthesis of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-(1,3-dioxan-2-yl)-2-methoxybenzoate

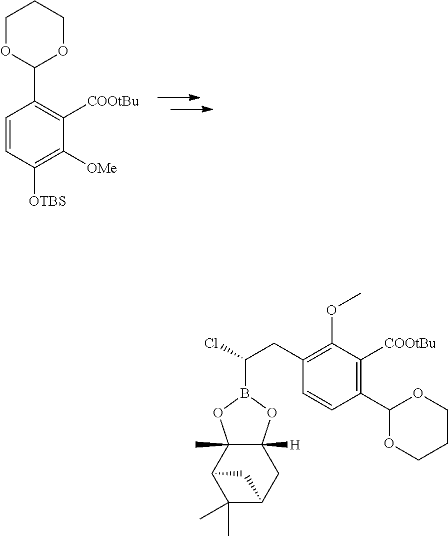

By following the procedures described in Step 3-Step 7 of Example 15, the above product from Step 6 was converted to the title compound. ESI-MS m/z 557 (M+Na)⁺.

### Step 8. Synthesis of (3R)-3-(2-(2-aminothiazol-4-yl)-2-(4-ethyl-2,3-dioxopiperazine-1-carboxamido)acetamido)-7-formyl-2-hydroxy-3,4-dihydro-2H-benzo[e][1,2]oxaborinine-8-carboxylic acid

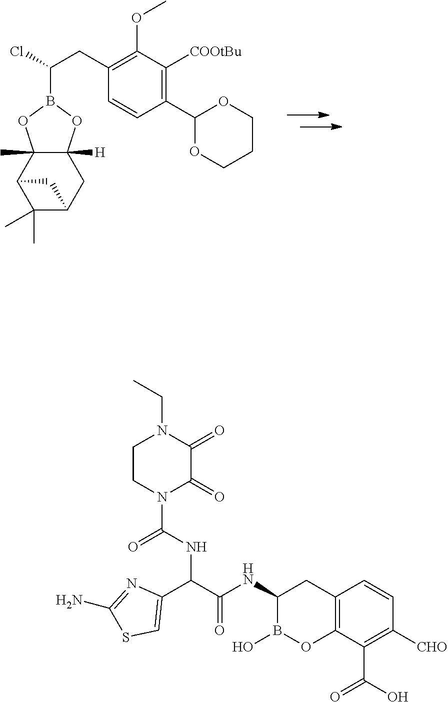

The title compound was prepared from the above chloride from Step 7, and the carboxylic acid from Step 5 by following the General coupling method C, and utilizing BBr₃ in the final deprotection reaction. ESIMS m/z 559 (M+1)⁺.

### Example 58: (3R)-3-(2-(2-aminothiazol-4-yl)-2-(4-ethyl-2-hydroxy-3-oxopiperazine-1-carboxamido)acetamido)-2-hydroxy-7-(hydroxymethyl)-3,4-dihydro-2H-benzo[el][1,2]oxaborinine-8-carboxylic acid

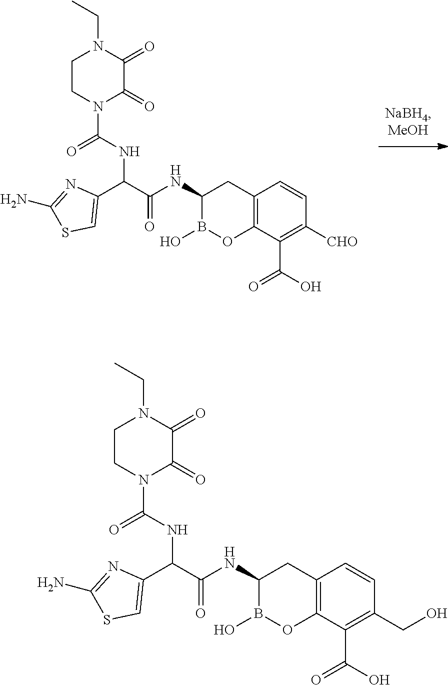 [SMILES: CCN1CCN(C(=O)NC(C(=O)N[C@H]2Cc3ccc(C=O)c(C(=O)O)c3OB2O)c2csc(N)n2)C(=O)C1=O ; CCN1CCN(C(=O)NC(C(=O)N[C@H]2Cc3ccc(CO)c(C(=O)O)c3OB2O)c2csc(N)n2)C(=O)C1=O]

To a solution of Example 57 (19 mg, 0.034 mmol) in MeOH (1 mL) was added NaBH₄ (3 mg, 0.079 mmol). After 5 min, the reaction mixture was diluted with 0.5 mL of water, and subjected to reversed phase HPLC purification to yield the title compound after lyophilization. ESI-MS m/z 563 (M+H)⁺.

### Example 59: (3R)-7-(((3S,5S)-5-(dimethylcarbamoyl)pyrrolidin-3-yl)oxy)-3-(2-(4-ethyl-2,3-dioxopiperazine-1-carboxamido)-2-phenylacetamido)-2-hydroxy-3,4-dihydro-2H-benzo[e][1,2]oxaborinine-8-carboxylic acid

### Step 1. Synthesis of tert-butyl 6-(benzyloxy)-3-bromo-2-methoxybenzoate

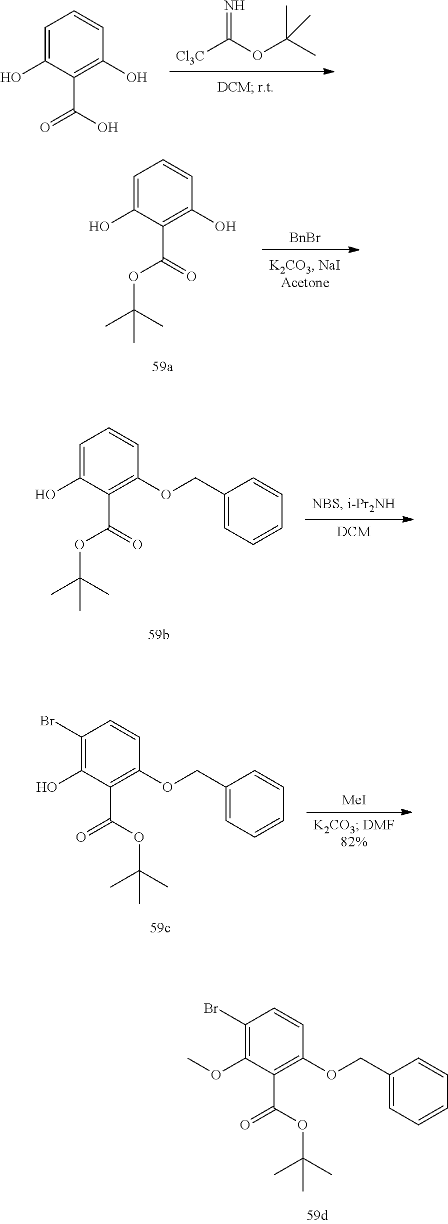

### Step 1a. Synthesis of tert-butyl 2,6-dihydroxybenzoate

2,6-dihydroxybenzoic acid (15.4 g, 100 mmol) was dissolved in 150 mL DCM, and tert-butyl 2,2,2-trichloroacetimidate (32.3 g, 150 mmol) was added, and the resulting mixture was stirred at ambient temperature. The mixture was filtered, the filtrate was diluted with hexanes to precipitate byproducts, and then the mixture was filtered again. The filtrate was concentrated in vacuo and purified by silica gel chromatography (0-5% EtOAc-hexane) to provide the title compound, 12.8 g (61.1 mmol, 61% yield). ESI-MS m/z 211.2 (M+H)⁺

### Step 1b. Synthesis of tert-butyl 2-(benzyloxy)-6-hydroxybenzoate

A solution of tert-butyl 2,6-dihydroxybenzoate (18.9 g, 87.5 mmol) in 250 mL acetone was treated with potassium carbonate (12.1 g, 87.5 mmol) and benzyl bromide (15 g, 187.5 mmol), and the resulting mixture was stirred for 30 min at room temperature, and then at 50° C. for 24 h. The mixture was filtered, the filtrate was concentrated in vacuo, and the residue was purified by silica gel chromatography to provide title compound, 5.7 g (19.0 mmol, 22% yield) of ˜75% pure material.

### Step 1c. Synthesis of tert-butyl 6-(benzyloxy)-3-bromo-2-hydroxybenzoate

A solution of the product from Step 1b (5.12 g, 17 mmol) and diisopropylamine (337 mg, 3.33 mmol) in 15 mL DCM was cooled to −78° C. (Ar), and N-bromosuccinimide (3.19 g, 17.9 mmol) was added portionwise over 30 min. The reaction was allowed to warm to room temperature and stir overnight. The mixture was concentrated in vacuo and the residue was purified by silica gel chromatography (hexane-DCM, 0-10%) to provide 6.40 g (80%) of the title compound. ESI-MS m/z 401, 403 (M+Na)⁺.

### Step 1d. Synthesis of tert-butyl 6-(benzyloxy)-3-bromo-2-methoxybenzoate

A solution of the product from Step 1c (10.8 g, 28.5 mmol) in 50 mL DMF (Ar) was treated with anhydrous potassium carbonate (5.12 g, 37 mmol), then methyl iodide (525 g, 2.30 mmol) was added, and the resulting mixture was stirred at room temperature for 18 h. The mixture was partitioned between water and diethyl ether, the organic phase was washed with water, then with brine, dried (Na₂SO₄), concentrated in vacuo, and the residue purified by silica gel chromatography (DCM-hexanes, 0-35%) to provide the title compound (9.0 g, 80%). ESI-MS m/z 392, 394 (M+H)⁺.

### Step 2. Synthesis of tert-butyl 6-(benzyloxy)-3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate

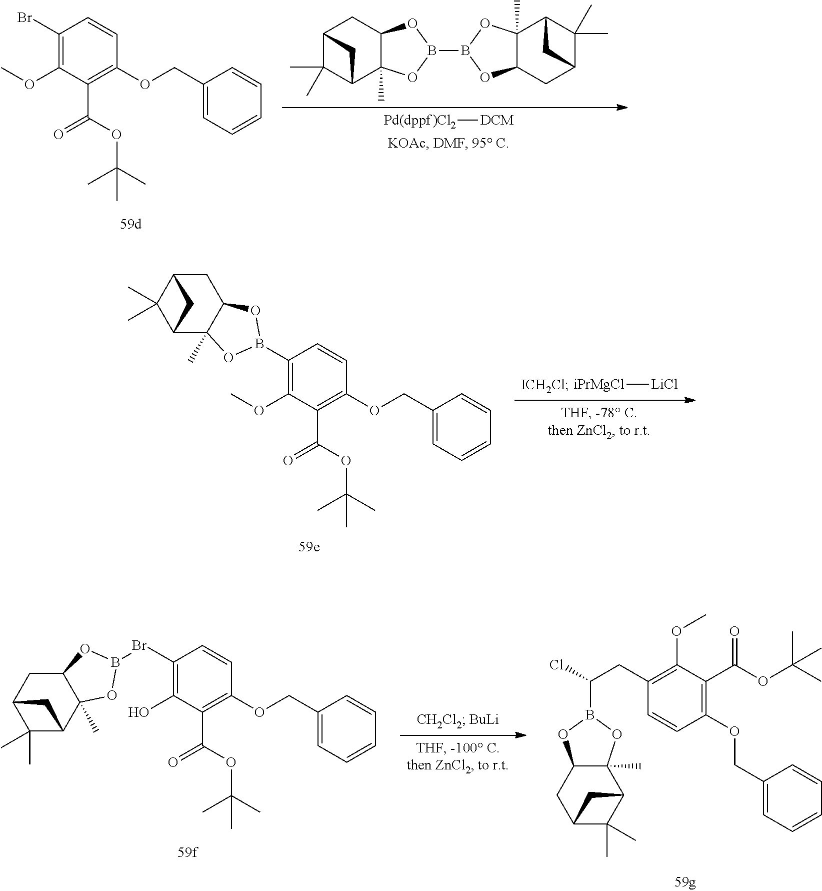

### Step 2a. Synthesis of tert-butyl 6-(benzyloxy)-2-methoxy-3-((3aS,4S,6S,7aR)-3a,5,5-trimethylhexahydro-4,6-methanobenzo[d][1,3,2]dioxaborol-2-yl)benzoate

In a manner analogous to that described in WO2014151958, a solution of tert-butyl 6-(benzyloxy)-3-bromo-2-methoxybenzoate (550 mg, 1.40 mmol) in 5 mL DMF was treated with (3aS,3a'S,4S,4'S,6S,6'S,7aR,7a′R)-2,2′-Bi-4,6-methano-1,3,2-benzodioxaborole, dodecahydro-3a,3′a,5,5,5′,5′-hexamethyl) (751 mg, 2.10 mmol), Pd(dppf)Cl2-DCM complex (57 mg, 0.07 mmol), and potassium acetate 412 mg, 4.20 mmol). The resulting mixture was heated at 80° C. for 18 hr. The reaction mixture was cooled to room temperature, then partitioned between diethyl ether and water, and the organic phase was concentrated in vacuo. The crude product was purified by silica gel chromatography to provide the title compound (570 mg, 83%). ESI-MS m/z 493 (M+H)⁺.

### Step 2b. Synthesis of tert-butyl 6-(benzyloxy)-2-methoxy-3-(((3aS,4S,6S,7aR)-3a,5,5-trimethylhexahydro-4,6-methanobenzo[d][1,3,2]dioxaborol-2-yl)methyl)benzoate

A solution of iodochloromethane (9.28 g, 52.6 mmol) in 25 mL THF (Ar) was cooled to −78° C. and treated dropwise with a solution of isopropylmagnesium chloride-lithium chloride complex (20.3 mL, 1.3 M in THF) over 30 min. The resulting solution was stirred an additional 45 min at −78° C. A solution of the product from Step 2a (4.32 g, 8.78 mmol) in 8 mL THF was then added dropwise over 30 min, and the resulting solution was stirred an additional 2.5 h. A solution of zinc chloride (17.6 mL, 1 M THF) was added dropwise over 30 min, and the resulting mixture was stirred an additional 15 min at −78° C. The cooling bath was removed, and the mixture was allowed to warm to room temperature and was stirred overnight. The reaction mixture was partitioned between diethyl ether and water, the organic phase was washed with brine, then dried (MgSO₄), concentrated in vacuo, and the residue was purified by silica gel chromatography (0-10% EtOAc-hexane) to yield the title compound (4.28 g, 96%). ESI-MS m/z 528 (M+H)⁺.

### Step 2c. Synthesis of tert-butyl 6-(benzyloxy)-3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate

The procedure of WO 2014/089365 was adapted, using 61f in place of tert-butyl 2-methoxy-3-(((3aS,4S,6S,7aR)-3a,5,5-trimethylhexahydro-4,6-methanobenzo[d][1,3,2]dioxaborol-2-yl)methyl)benzoate. ESI-MS m/z 555 (M+H)⁺.

### Step 3. Synthesis of (3R)-7-(((3S,5S)-5-(dimethylcarbamoyl)pyrrolidin-3-yl)oxy)-3-(2-(4-ethyl-2,3-dioxopiperazine-1-carboxamido)-2-phenylacetamido)-2-hydroxy-3,4-dihydro-2H-benzo[e][1,2]oxaborinine-8-carboxylic acid

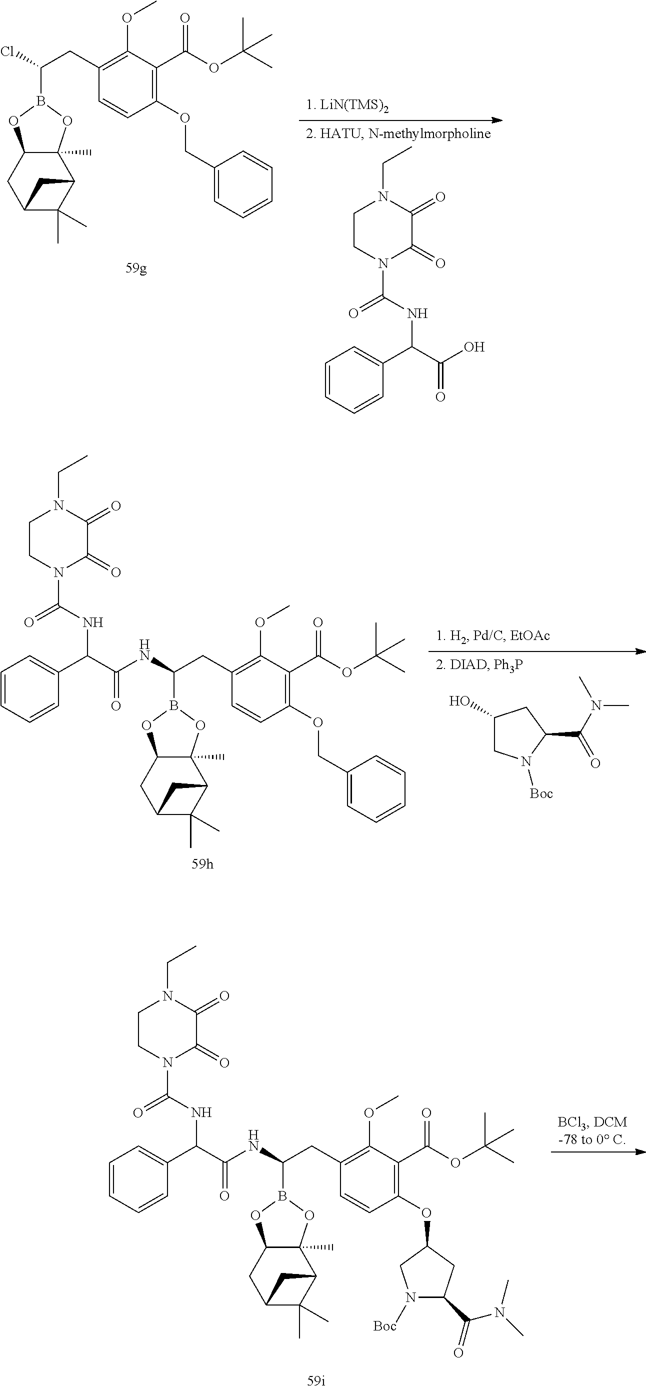

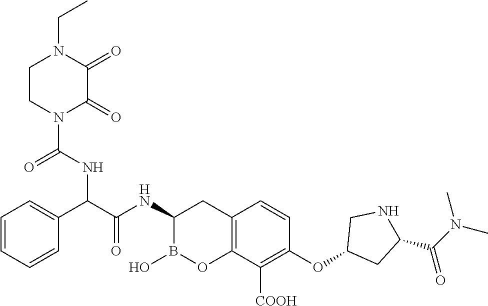 [SMILES: CCN1CCN(C(=O)NC(C(=O)N[C@H]2Cc3ccc(O[C@@H]4CN[C@H](C(=O)N(C)C)C4)c(C(=O)O)c3OB2O)c2ccccc2)C(=O)C1=O]

### Step 3a. Synthesis of tert-butyl 6-(benzyloxy)-3-((2R)-2-(2-(4-ethyl-2,3-dioxopiperazine-1-carboxamido)-2-phenylacetamido)-2-((3aS,4S,6S,7aR)-3a,5,5-trimethylhexahydro-4,6-methanobenzo[d][1,3,2]dioxaborol-2-yl)ethyl)-2-methoxybenzoate

General Method C was utilized to convert the product from Step 2c to the title compound.

### Step 3b. Synthesis of tert-butyl (2S,4S)-4-(2-(tert-butoxycarbonyl)-4-((2R)-2-(2-(4-ethyl-2,3-dioxopiperazine-1-carboxamido)-2-phenylacetamido)-2-((3aS,4S,6S,7aR)-3a,5,5-trimethylhexahydro-4,6-methanobenzo[d][1,3,2]dioxaborol-2-yl)ethyl)-3-methoxyphenoxy)-2-(dimethylcarbamoyl)pyrrolidine-1-carboxylate

A solution of the product from Step 3a (400 mg, 0.489 mmol) in 10 mL EtOAc was added to 10% palladium on carbon (300 mg), the solution was placed under vacuum, then and stirred under 1 atm hydrogen gas for 24 h. The mixture was placed under vacuum to remove hydrogen gas, then argon was introduced to the mixture. The mixture was then filtered through Celite, the filtrate was concentrated in vacuo, and the reside was used directly. ESI-MS m/z 691.3 (M+H-isobutene).

The crude product dissolved in 5 mL DCM, then tert-butyl (2S,4S)-2-(dimethylcarbamoyl)-4-hydroxypyrrolidine-1-carboxylate (139 mg, 0.535 mmol) and triphenylphosphine (140 mg, 0.534 mmol) was added, and the resulting solution was cooled to 0° C. (Ar), and diisopropyl azodicarboxylate (0.106 mL, 108 mg, 0.536 mmol) was added dropwise. The ice bath was removed and the resulting mixture was stirred overnight at room temperature. Additional portions of tert-butyl (2S,4S)-2-(dimethylcarbamoyl)-4-hydroxypyrrolidine-1-carboxylate (55.4 mg, 0.213 mmol) and triphenylphosphine (56.2 mg, 0.214 mmol) were added to the reaction mixture, followed by an additional portion of diisopropyl azodicarboxylate (43 mg, 0.213 mmol), and the mixture was stirred an additional 18 h. The mixture was concentrated and purified by silica gel chromatography to provide the title compound (234 mg, 43%), which contained ˜22% triphenylphosphine oxide.

### Step 3c. Synthesis of (3R)-7-(((3S,5S)-5-(dimethylcarbamoyl)pyrrolidin-3-yl)oxy)-3-(2-(4-ethyl-2,3-dioxopiperazine-1-carboxamido)-2-phenylacetamido)-2-hydroxy-3,4-dihydro-2H-benzo[e][1,2]oxaborinine-8-carboxylic acid

A solution of the product from Step 3b (192 mg, ˜78% purity, 0194 mmol) in 4 mL DCM was cooled to −78° C., and boron trichloride (1.1 mL, 1 M in DCM, 1.17 mmol) was added. The reaction was allowed to warm to 0° C. and was stirred 30 min. Methanol (1 mL) and water (1 mL) were added to the mixture, and the resulting mixture was stirred for 15 min. The mixture was concentrated in vacuo, and the reside was purified by reverse-phase HPLC (water-MeCN with 0.1% TFA) to provide the title compound as a white solid TFA salt (37 mg, 24%). ESI-MS m/z 665.3 (M+H)⁺.

### Example 60: (3R)-7-(2-aminoethoxy)-3-(2-(4-ethyl-2,3-dioxopiperazine-1-carboxamido)-2-phenylacetamido)-2-hydroxy-3,4-dihydro-2H-benzo[e][1,2]oxaborinine-8-carboxylic acid

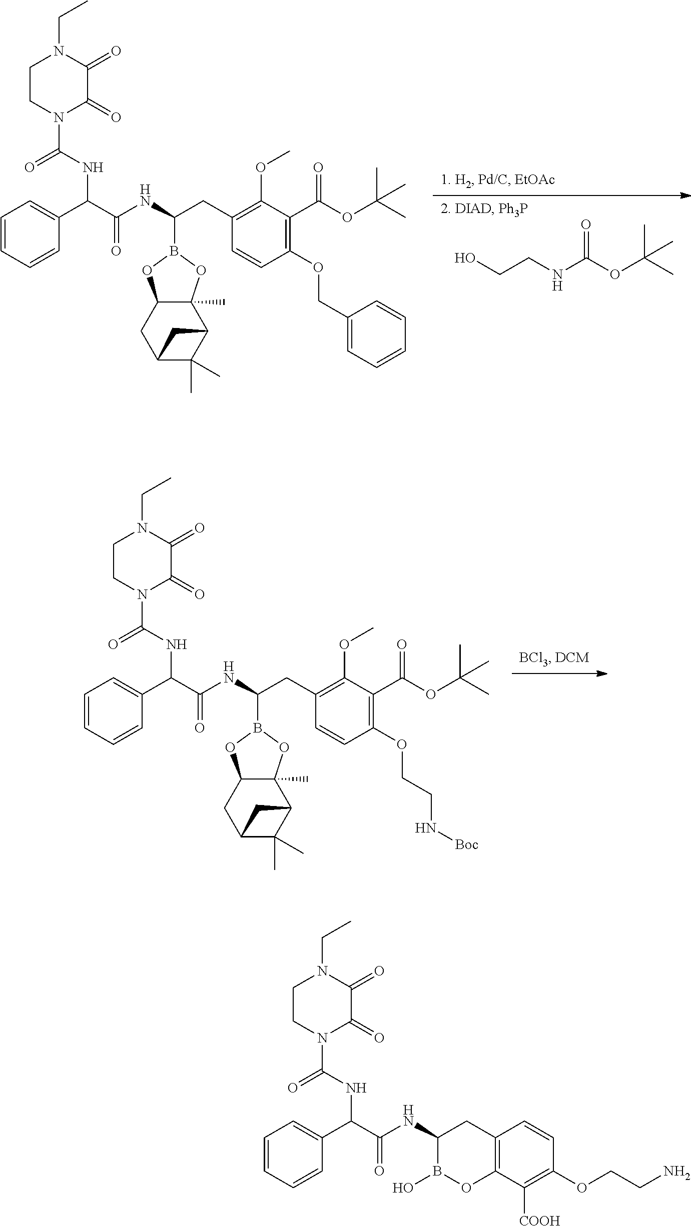

### Step 1. Synthesis of tert-butyl 6-(2-((tert-butoxycarbonyl)amino)ethoxy)-3-((2R)-2-(2-(4-ethyl-2,3-dioxopiperazine-1-carboxamido)-2-phenylacetamido)-2-((3aS,4S,6S,7aR)-3a,5,5-trimethylhexahydro-4,6-methanobenzo[d][1,3,2]dioxaborol-2-yl)ethyl)-2-methoxybenzoate

The procedures of Example 59, Steps 3a and 3b were utilized, with the substitution of tert-butyl (2-hydroxyethyl)carbamate for tert-butyl (2S,4S)-2-(dimethylcarbamoyl)-4-hydroxypyrrolidine-1-carboxylate, to provide the title compound. ESI-MS m/z 790.4 (M+H)⁺.

### Step 2. Synthesis of (3R)-7-(2-aminoethoxy)-3-(2-(4-ethyl-2,3-dioxopiperazine-1-carboxamido)-2-phenylacetamido)-2-hydroxy-3,4-dihydro-2H-benzo[e][1,2]oxaborinine-8-carboxylic acid

The procedure of Example 59, Step 3c was utilized to convert the product from Example 63, Step 1 to provide the title compound. ESI-MS m/z 568.2 (M+H)⁺.

### Example 61: (3R)-3-(2-(4-ethyl-2,3-dioxopiperazine-1-carboxamido)-2-phenylacetamido)-2,7-dihydroxy-3,4-dihydro-2H-benzo[e][1,2]oxaborinine-8-carboxylic acid

Example 61 was isolated as a less polar byproduct during the purification of Example 60, Step 2 (1.8 mg obtained). ESI-MS m/z 525.3 (M+H)⁺.

### Example 62: (3R)-7-(((2-aminoethoxy)imino)methyl)-3-(2-(2-aminothiazol-4-yl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of tert-butyl 3-((2R)-2-(2-(2-(((benzyloxy)carbonyl)amino)thiazol-4-yl)-2-(4-ethyl-2,3-dioxopiperazine-1-carboxamido)acetamido)-2-((3aS,4S,6S,7aR)-3a,5,5 trimethylhexahydro-4,6-methanobenzo[d][1,3,2]dioxaborol-2-yl)ethyl)-6-formyl-2-methoxybenzoate

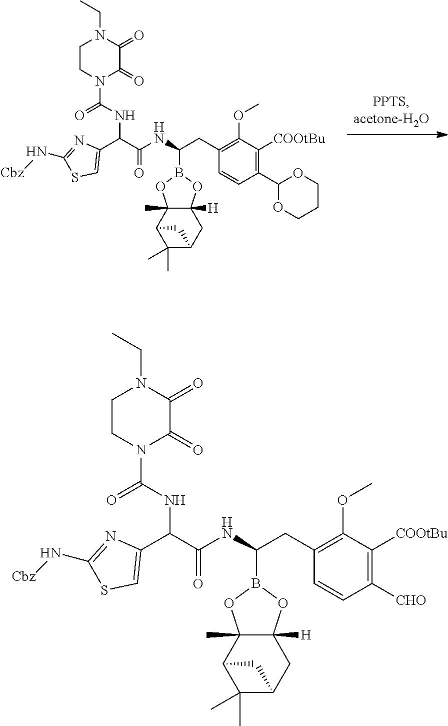

The acetal intermediate from the synthesis of Example 57 (4 g, 4.1 mml) was dissolved in acetone (136 mL) and water (7 mL), and refluxed in the presence of PPTS (251 mg, 1 mmol) for 24 h, then concentrated, and purified by flash chromatography on silica gel (hexane-acetone, 4:1-2:3) to afford the title compound, 2.08 g. ESI-MS m/z 915 (M+1)⁺.

### Step 2. Synthesis of tert-butyl 3-((2R)-2-(2-(2-(((benzyloxy)carbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-(9,9-dimethyl-7-oxo-3,8-dioxa-2,6-diazadec-1-en-1-yl)-2-methoxybenzoate

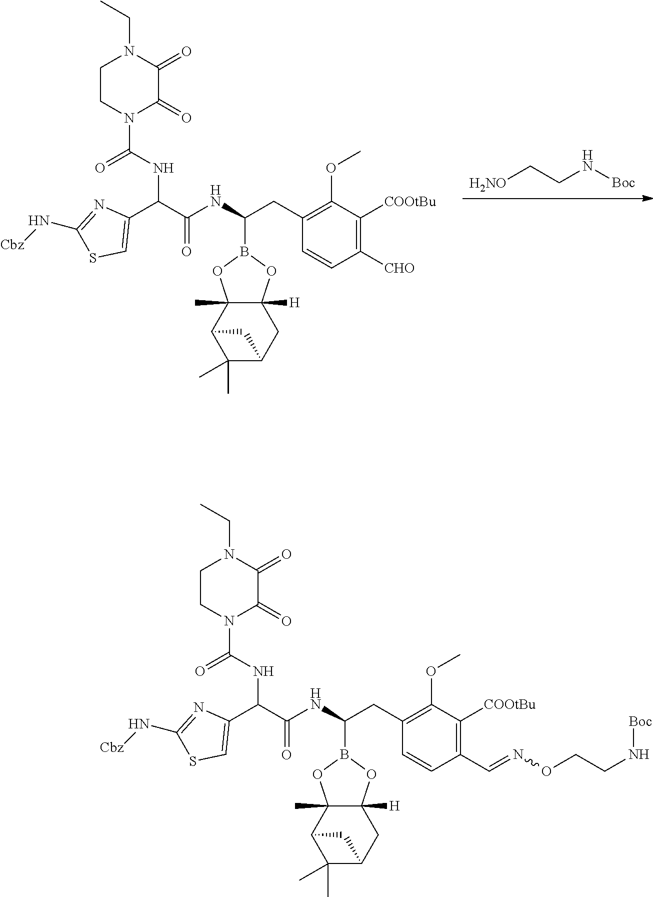

To the above product (366 mg, 0.4 mmol) in MeOH (6 mL) was added tert-butyl 2-(aminooxy)ethylcarbamate (17 mg, 0.4 mmol). The reaction mixture was stirred at RT for 3 h, then concentrated in vacuo to afford the crude product, which was used directly for the next step without further purification. ESI-MS m/z 1073 (M+1)⁺.

### Step 3. Synthesis of (3R)-7-(((2-aminoethoxy)imino)methyl)-3-(2-(2-aminothiazol-4-yl)-2-(4-ethyl-2,3-dioxopiperazine-1-carboxamido)acetamido)-2-hydroxy-3,4-dihydro-2H-benzo[e][1,2]oxaborinine-8-carboxylic acid

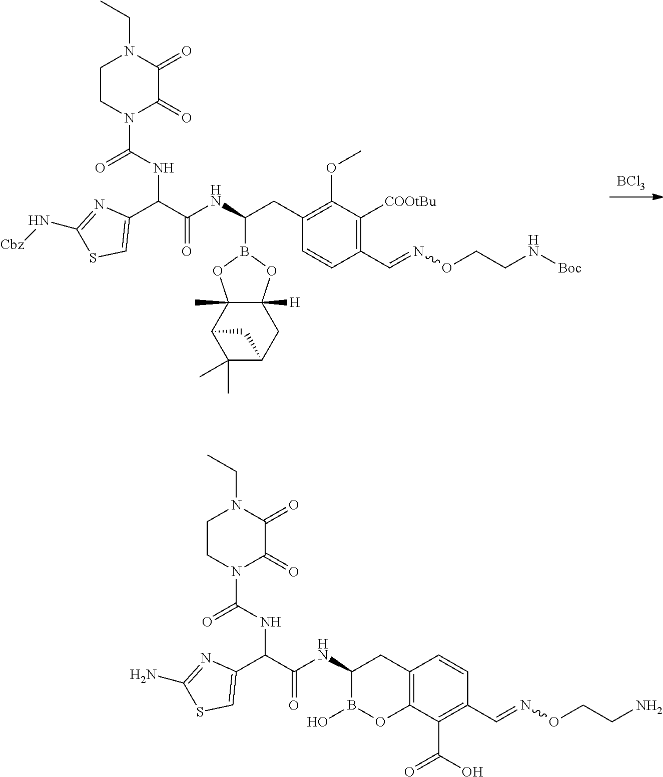

The title compound was prepared by deprotection of the above crude product with BCl₃. ESI-MS m/z 617 (M+1)⁺.

### Example 63: (3R)-3-(2-(2-aminothiazol-4-yl)-2-(4-ethyl-2,3-dioxopiperazine-1-carboxamido)acetamido)-2-hydroxy-7-(trifluoromethyl)-3,4-dihydro-2H-benzo[e][1,2]oxaborinine-8-carboxylic acid

### Step 1. Synthesis of tert-butyl 2-((tert-butoxycarbonyl)oxy)-6-(trifluoromethyl)-3-(((3aS,4S,6S,7aR)-3a,5,5-trimethylhexahydro-4,6-methanobenzo[d][1,3,2]dioxaborol-2-yl)methyl)benzoate

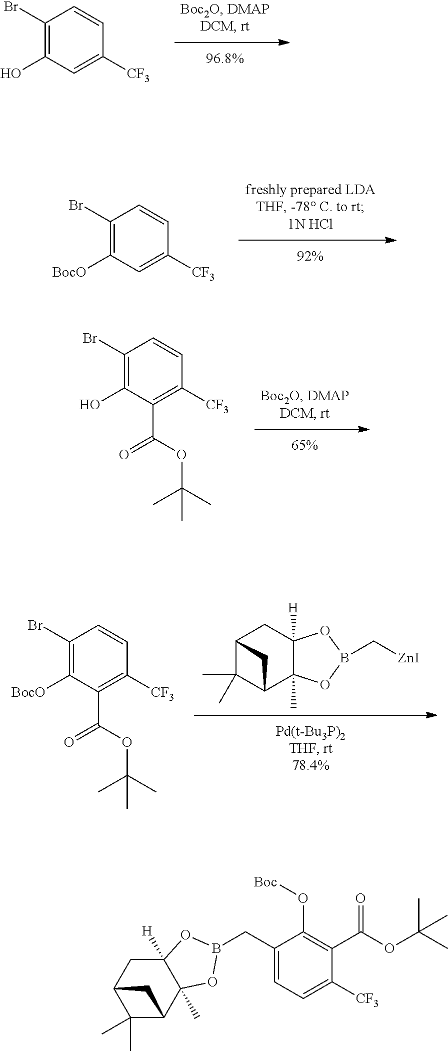

To a mixture of 2-bromo-5-(trifluoromethyl)phenol (15.00 g, 62.2 mmol) and Boc₂O (16.30 g, 74.7 mmol) in DCM (200 mL) was added DMAP (0.570 g, 4.66 mmol) at room temperature. The reaction mixture was stirred at room temperature for 24 h. After the solvent was evaporated under reduced pressure, the residue was purified by flash chromatography on silica gel (340 g column) eluted with 0 to 15% ethyl acetate/hexanes to afford 20.56 g (96.8%) of 2-bromo-5-(trifluoromethyl)phenyl tert-butyl carbonate. ESI-MS m/z 283.1, 285.2 (M-56)⁺.

To a solution of 2-bromo-5-(trifluoromethyl)phenyl tert-butyl carbonate (20.56 g, 60.3 mmol) in THF (150 mL) at −78° C. under argon was added freshly prepared LDA, n-BuLi (2.5 M in hexane, 29 mL, 72.5 mmol) was added dropwise to a solution of DIPA (10.2 mL, 72.8 mmol) in THF (100 mL) at −78° C. under argon and the resulting solution was stirred at −78° C. for 0.5 h and then 0° C. for 1 h, via cannula. The reaction mixture was allowed to slowly warm to room temperature overnight and then quenched with 1 N HCl (160 mL), extracted with ethyl acetate (3×), dried over Na₂SO₄, and evaporated under reduced pressure, the residue was purified by flash chromatography on silica gel (340 g column) eluted with 0 to 20% ethyl acetate/hexanes to afford 18.90 g (92%) of tert-butyl 3-bromo-2-hydroxy-6-(trifluoromethyl)benzoate.

To a mixture of tert-butyl 3-bromo-2-hydroxy-6-(trifluoromethyl)benzoate (18.90 g, 55.4 mmol) and Boc₂O (18.43 g, 84.4 mmol) in DCM (200 mL) was added DMAP (0.726 g, 5.94 mmol) at room temperature. The reaction mixture was stirred at room temperature for 17 h. After the solvent was evaporated under reduced pressure, the residue was purified by flash chromatography on silica gel (340 g column) eluted with 0 to 20% ethyl acetate/hexanes to afford 15.89 g (65%) of tert-butyl 3-bromo-2-((tert-butoxycarbonyl)oxy)-6-(trifluoromethyl)benzoate as a solid. ESI-MS m/z 905.1, 907.0 (2M+Na)⁺.

To a solution of tert-butyl 3-bromo-2-((tert-butoxycarbonyl)oxy)-6-(trifluoromethyl)benzoate (15.87 g, 36.0 mmol) and bis(tri-tert-butylphosphine)palladium(0) (1.092 g, 2.14 mmol) in THF (40 mL) at room temperature under argon was added a solution of (((3aS,4S,6S,7aR)-3a,5,5-trimethylhexahydro-4,6-methanobenzo[d][1,3,2]dioxaborol-2-yl)methyl)zinc(II) iodide (0.5 M in THF, 156 mL, 78.0 mmol), prepared as described in Example 59 step 3. The resulting mixture was stirred at room temperature for 22 h. After the solvent was evaporated under reduced pressure, the residue was purified by flash chromatography on silica gel (340 g column) eluted with 0 to 20% ethyl acetate/hexanes to afford 15.64 g (78.4%) of tert-butyl 2-((tert-butoxycarbonyl)oxy)-6-(trifluoromethyl)-3-(((3aS,4S,6S,7aR)-3a,5,5-trimethylhexahydro-4,6-methanobenzo[d][1,3,2]dioxaborol-2-yl)methyl)benzoate. ESI-MS m/z 1131.5 (2M+Na)⁺.

### Step 2. Synthesis of tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-(trifluoromethyl)benzoate

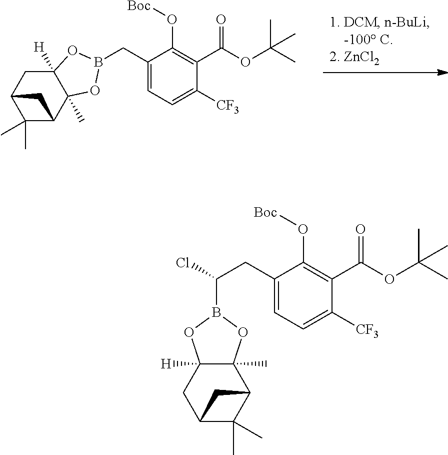 [SMILES: CC(C)(C)OC(=O)Oc1c(CB2O[C@@H]3C[C@@H]4C[C@@H](C4(C)C)[C@]3(C)O2)ccc(C(F)(F)F)c1C(=O)OC(C)(C)C ; CC(C)(C)OC(=O)Oc1c(C[C@@H](Cl)B2O[C@@H]3C[C@@H]4C[C@@H](C4(C)C)[C@]3(C)O2)ccc(C(F)(F)F)c1C(=O)OC(C)(C)C]

The Matteson reaction was carried out as described in Example 54 step 1 by using tert-butyl 2-((tert-butoxycarbonyl)oxy)-6-(trifluoromethyl)-3-(((3aS,4S,6S,7aR)-3a,5,5-trimethylhexahydro-4,6-methanobenzo[d][1,3,2]dioxaborol-2-yl)methyl)benzoate as the substrate to afford tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-(trifluoromethyl)benzoate. ESI-MS m/z 625.3 (M+Na)⁺.

### Step 3. Synthesis of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-(trifluoromethyl)benzoate

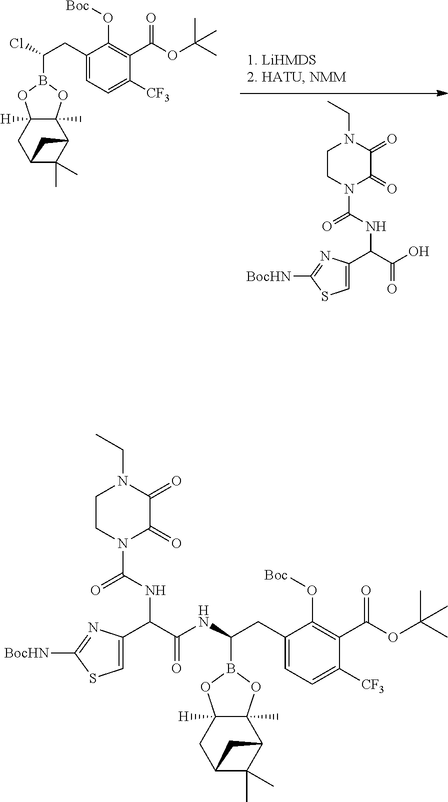 [SMILES: CC(C)(C)OC(=O)Oc1c(C[C@@H](Cl)B2O[C@@H]3C[C@@H]4C[C@@H](C4(C)C)[C@]3(C)O2)ccc(C(F)(F)F)c1C(=O)OC(C)(C)C ; CCN1CCN(C(=O)NC(C(=O)O)c2csc(NC(=O)OC(C)(C)C)n2)C(=O)C1=O ; CCN1CCN(C(=O)NC(C(=O)N[C@@H](Cc2ccc(C(F)(F)F)c(C(=O)OC(C)(C)C)c2OC(=O)OC(C)(C)C)B2O[C@@H]3C[C@@H]4C[C@@H](C4(C)C)[C@]3(C)O2)c2csc(NC(=O)OC(C)(C)C)n2)C(=O)C1=O]

The amide formation was carried out as described in Example 56 Step 5 by using tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-(trifluoromethyl)benzoate as the substrate to afford tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-(trifluoromethyl)benzoate. ESI-MS m/z 1007.4 (M+H)⁺.

### Step 4. Synthesis of (3R)-3-(2-(2-aminothiazol-4-yl)-2-(4-ethyl-2,3-dioxopiperazine-1-carboxamido)acetamido)-2-hydroxy-7-(trifluoromethyl)-3,4-dihydro-2H-benzo[e][1,2]oxaborinine-8-carboxylic acid

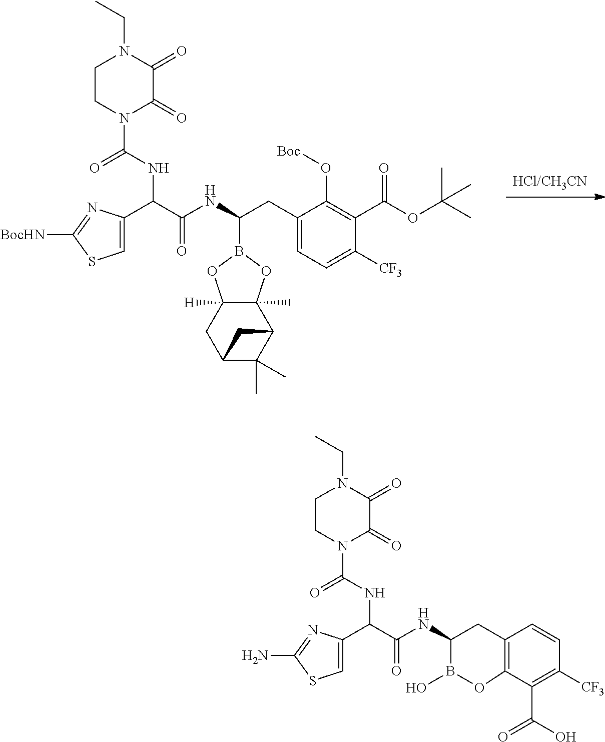 [SMILES: CCN1CCN(C(=O)NC(C(=O)N[C@@H](Cc2ccc(C(F)(F)F)c(C(=O)OC(C)(C)C)c2OC(=O)OC(C)(C)C)B2O[C@@H]3C[C@@H]4C[C@@H](C4(C)C)[C@]3(C)O2)c2csc(NC(=O)OC(C)(C)C)n2)C(=O)C1=O ; CCN1CCN(C(=O)NC(C(=O)N[C@H]2Cc3ccc(C(F)(F)F)c(C(=O)O)c3OB2O)c2csc(N)n2)C(=O)C1=O]

A mixture of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-(trifluoromethyl)benzoate (0.36 g), CH₃CN (24 mL), and 6 N HCl (6 mL) was stirred at room temperature for 23 h. The mixture was lyophilized, purified by reverse phase HPLC (Gilson, C18 column) and then lyophilized to give (3R)-3-(2-(2-aminothiazol-4-yl)-2-(4-ethyl-2,3-dioxopiperazine-1-carboxamido)acetamido)-2-hydroxy-7-(trifluoromethyl)-3,4-dihydro-2H-benzo[e][1,2]oxaborinine-8-carboxylic acid as TFA salt. ESI-MS m/z 599.1 (M+H)⁺.

### Example 64: (3R)-3-(2-(2-aminothiazol-4-yl)-2-(4-ethyl-2,3-dioxopiperazine-1-carboxamido)acetamido)-2-hydroxy-7-((2-(pyrrolidin-1-yl)ethyl)thio)-3,4-dihydro-2H-benzo[e][1,2]oxaborinine-8-carboxylic acid

### Step 1. Synthesis of sodium 2,2-diethoxyethane-1-thiolate

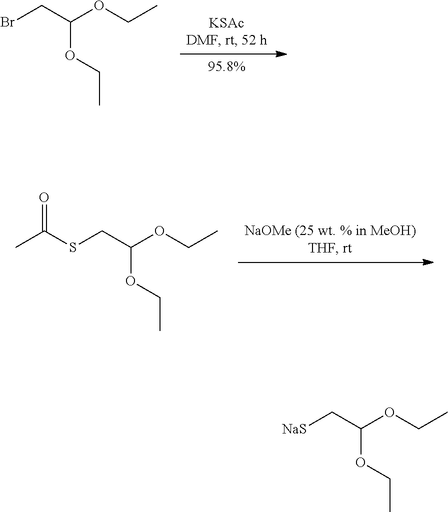

A mixture of 2-bromo-1,1-diethoxyethane (27.40 g, 139 mmol) and KSAc (20.10 g, 176 mmol) in DMF (60 mL) was stirred at room temperature for 52 h. The reaction mixture was then quenched with water, extracted with ethyl acetate (3×). The combined organic phase was dried over Na₂SO₄. After the solvent was evaporated under reduced pressure, the residue was purified by flash chromatography on silica gel (340 g column) eluted with 0 to 20% ethyl acetate/hexanes to afford 25.61 g (95.8%) of S-(2,2-diethoxyethyl) ethanethioate. ESI-MS m/z 147.0 (M-OEt)⁺.

To a solution of S-(2,2-diethoxyethyl) ethanethioate (25.61 g, 133 mmol) in THF (100 mL) was added NaOMe (25 wt. % in MeOH, 29.06 g, 134.5 mmol) dropwise at 0° C. and then stirred at room temperature overnight, which was directly used in the next step.

### Step 2. Synthesis of tert-butyl 3-bromo-2-((tert-butoxycarbonyl)oxy)-6-((2,2-diethoxyethyl)thio)benzoate

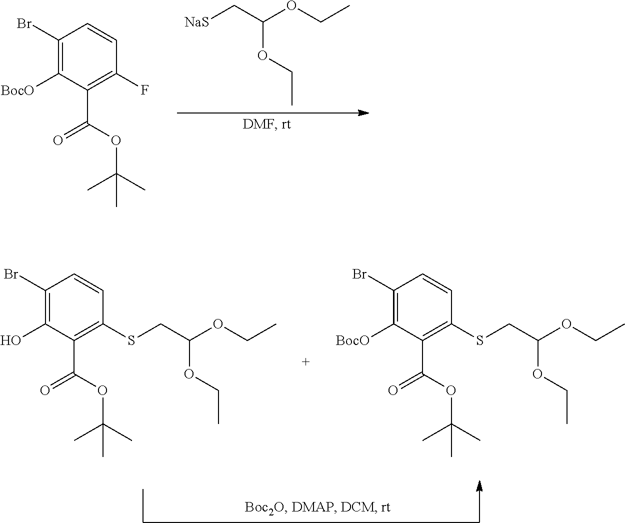

To a solution of tert-butyl 3-bromo-2-((tert-butoxycarbonyl)oxy)-6-fluorobenzoate (37.10 g, 94.8 mmol) in DMF (120 mL) at room temperature was added sodium 2,2-diethoxyethane-1-thiolate (133 mmol), prepared as described above, via cannula. The resulting mixture was stirred at room temperature for 4 d. The reaction mixture was then quenched with water, extracted with ethyl acetate (3×). The combined organic phase was dried over Na₂SO₄. After the solvent was evaporated under reduced pressure, the residue was purified by flash chromatography on silica gel (340 g column) eluted with 0 to 20% ethyl acetate/hexanes to afford 9.88 g (20%) of tert-butyl 3-bromo-2-((tert-butoxycarbonyl)oxy)-6-((2,2-diethoxyethyl)thio)benzoate, ESI-MS m/z 543.1, 545.1 (M+Na)⁺, and 9.90 g of tert-butyl 3-bromo-6-((2,2-diethoxyethyl)thio)-2-hydroxybenzoate, ESI-MS m/z 443.0, 445.0 (M+Na)⁺, which was converted into 9.06 g (18%) of tert-butyl 3-bromo-2-((tert-butoxycarbonyl)oxy)-6-((2,2-diethoxyethyl)thio)benzoate as described in Example 56 step 1.

### Step 3. Synthesis of (((3aS,4S,6S,7aR)-3a,5,5-trimethylhexahydro-4,6-methanobenzo[d][1,3,2]dioxaborol-2-yl)methyl)zinc(II) iodide

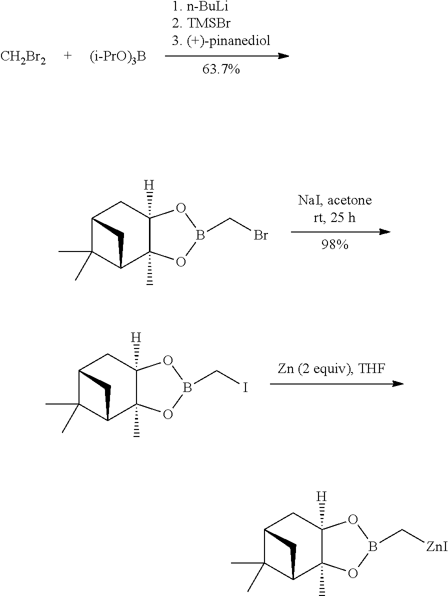

To a solution of dibromomethane (47.20 g, 271 mmol) and triisopropyl borate (39.85 g, 212 mmol) in THF (200 mL) at −78° C. under argon was added n-BuLi (2.5 M in hexane, 93.5 mL, 234 mmol) dropwise over 50 min. After the resulting mixture was stirred at −78° C. for 1.5 h, bromotrimethylsilane (31 mL, 235 mmol) was added dropwise. The reaction mixture was allowed to slowly warm to room temperature overnight. To the reaction mixture was then added (+)-pinanediol (36.03 g, 212 mmol) and stirred at room temperature for 4 h. The reaction mixture was then quenched with water, extracted with ethyl acetate (3×). The combined organic phase was dried over Na₂SO₄. After the solvent was evaporated under reduced pressure, the residue was purified by flash chromatography on silica gel (340 g column) eluted with 0 to 20% ethyl acetate/hexanes to afford 36.84 g (63.7%) of (3aS,4S,6S,7aR)-2-(bromomethyl)-3a,5,5-trimethylhexahydro-4,6-methanobenzo[d][1,3,2]dioxaborole as a clear liquid. ESI-MS m/z 273.1, 275.0 (M+H)⁺.

To a solution of (3aS,4S,6S,7aR)-2-(bromomethyl)-3a,5,5-trimethylhexahydro-4,6-methanobenzo[d][1,3,2]dioxaborole (35.59 g, 130 mmol) in acetone (150 mL) was added NaI (30.20 g, 201 mmol) at room temperature. The resulting mixture was stirred at room temperature for 24 h under argon in dark. Hexane was then added to the reaction mixture to precipitate the salt, filtered and the solid was washed with hexane. After the filtrate was evaporated under reduced pressure, the residue was purified by flash chromatography on silica gel (340 g column) eluted with 0 to 20% ethyl acetate/hexanes to afford 40.96 g (98%) of (3aS,4S,6S,7aR)-2-(iodomethyl)-3a,5,5-trimethylhexahydro-4,6-methanobenzo[d][1,3,2]dioxaborole as a clear liquid.

To a mixture of zinc dust (14.55 g, 222 mmol) in THF (75 mL) at 90° C. under argon were added chlorotrimethylsilane (0.6 mL) and 1,2-dibromoethane (0.6 mL) dropwise. After the mixture was stirred at 90° C. for 30 min to activate zinc, (3aS,4S,6S,7aR)-2-(iodomethyl)-3a,5,5-trimethylhexahydro-4,6-methanobenzo[d][1,3,2]dioxaborole (33.71 g, 105 mmol) in THF (80 mL) was added dropwise over 20 min. The mixture was stirred at 90° C. for additional 40 min and then stirred at room temperature overnight. After the mixture was standing for several hours. The clear solution (˜175 mL) was used in the next step directly as a solution of (((3aS,4S,6S,7aR)-3a,5,5-trimethylhexahydro-4,6-methanobenzo[d][1,3,2]dioxaborol-2-yl)methyl)zinc(II) iodide (0.6 M in THF).

### Step 4. Synthesis of tert-butyl 2-((tert-butoxycarbonyl)oxy)-6-((2,2-diethoxyethyl)thio)-3-(((3aS,4S,6S,7aR)-3a,5,5-trimethylhexahydro-4,6-methanobenzo[d][1,3,2]dioxaborol-2-yl)methyl)benzoate

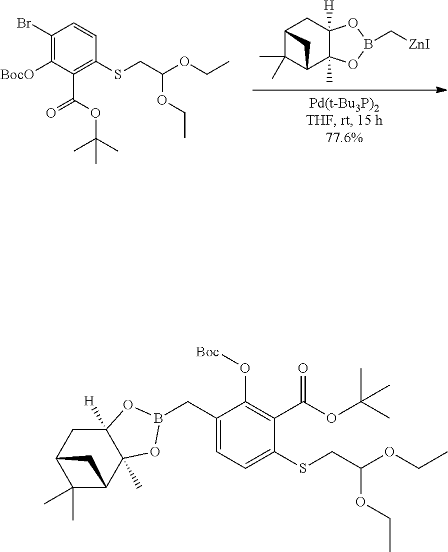

To a solution of tert-butyl 3-bromo-2-((tert-butoxycarbonyl)oxy)-6-((2,2-diethoxyethyl)thio)benzoate (9.80 g, 18.8 mmol) and bis(tri-tert-butylphosphine)palladium(0) (1.02 g, 2.0 mmol) in THF (60 mL) at room temperature under argon was added a solution of (((3aS,4S,6S,7aR)-3a,5,5-trimethylhexahydro-4,6-methanobenzo[d][1,3,2]dioxaborol-2-yl)methyl)zinc(II) iodide (0.6 M in THF, 102 mL, 61.2 mmol), prepared as described above. The resulting mixture was stirred at room temperature for 15 h. After the solvent was evaporated under reduced pressure, the residue was purified by flash chromatography on silica gel (340 g column) eluted with 0 to 20% ethyl acetate/hexanes to afford 9.26 g (77.6%) of tert-butyl 2-((tert-butoxycarbonyl)oxy)-6-((2,2-diethoxyethyl)thio)-3-(((3aS,4S,6S,7aR)-3a,5,5-trimethylhexahydro-4,6-methanobenzo[d][1,3,2]dioxaborol-2-yl)methyl)benzoate. ESI-MS m/z 657.3 (M+Na)⁺.

### Step 5. Synthesis of tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-((2,2-diethoxyethyl)thio)benzoate

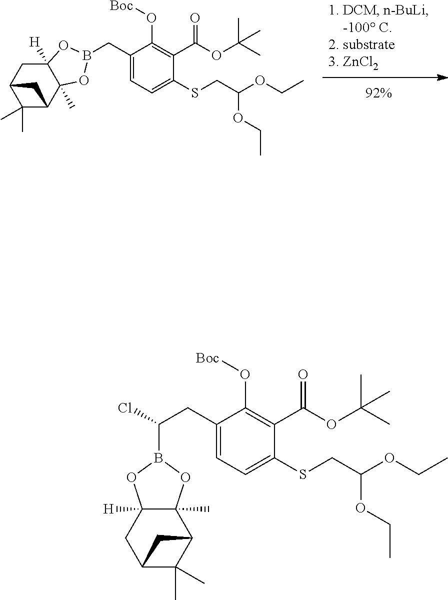 [SMILES: CCOC(CSc1ccc(CB2O[C@@H]3C[C@@H]4C[C@@H](C4(C)C)[C@]3(C)O2)c(OC(=O)OC(C)(C)C)c1C(=O)OC(C)(C)C)OCC ; "1. DCM, n-BuLi,    -100° C.2. substrate3. ZnCl2" ; CCOC(CSc1ccc(C[C@@H](Cl)B2O[C@@H]3C[C@@H]4C[C@@H](C4(C)C)[C@]3(C)O2)c(OC(=O)OC(C)(C)C)c1C(=O)OC(C)(C)C)OCC]

The Matteson reaction was carried out as described in Example 54 Step 1 by using tert-butyl 2-((tert-butoxycarbonyl)oxy)-6-((2,2-diethoxyethyl)thio)-3-(((3aS,4S,6S,7aR)-3a,5,5-trimethylhexahydro-4,6-methanobenzo[d][1,3,2]dioxaborol-2-yl)methyl)benzoate (8.11 g, 12.78 mmol) as the substrate to afford 8.02 g (92%) of tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-((2,2-diethoxyethyl)thio)benzoate. ESI-MS m/z 705.3 (M+Na)⁺.

### Step 6. Synthesis of 2-(2-((tert-butoxycarbonyl)amino)thiazol-4-yl)-2-(4-ethyl-2,3-dioxopiperazine-1-carboxamido)acetic acid

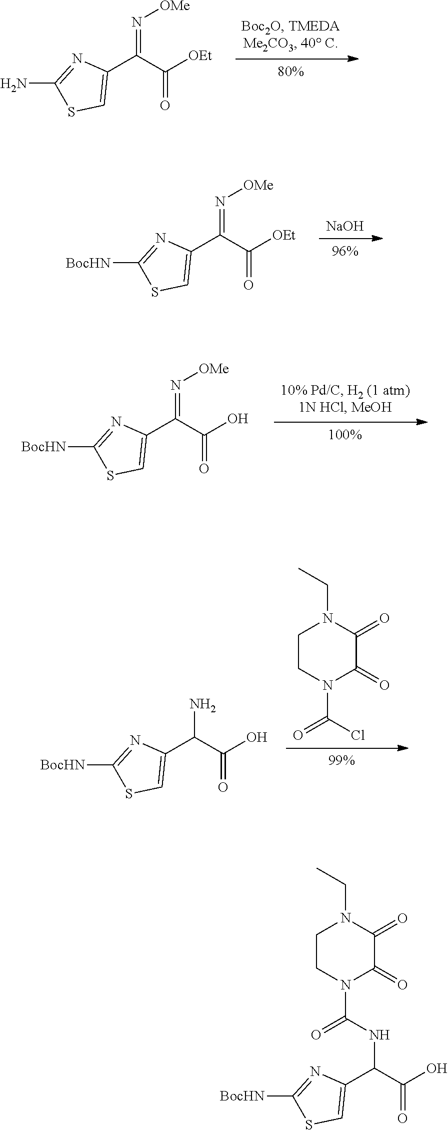

### Step 6a

A mixture of ethyl (Z)-2-(2-aminothiazol-4-yl)-2-(methoxyimino)acetate (14.80 g, 64.6 mmol), Boc₂O (15.49 g, 71.0 mmol), dimethyl carbonate (21 mL), and TMEDA (0.6 mL, 4.0 mmol) was stirred at 40° C. for 2 h. After the solvent was evaporated under reduced pressure, the residue was purified by flash chromatography on silica gel (340 g column) eluted with 0 to 40% ethyl acetate/hexanes to afford 17.02 g (80%) of ethyl (Z)-2-(2-((tert-butoxycarbonyl)amino)thiazol-4-yl)-2-(methoxyimino)acetate. ESI-MS m/z 330.1 (M+H)⁺.

### Step 6b

A mixture of ethyl (Z)-2-(2-((tert-butoxycarbonyl)amino)thiazol-4-yl)-2-(methoxyimino)acetate (17.02 g, 51.7 mmol) and NaOH (6.25 g, 156 mmol) in MeOH (40 mL) and water (40 mL) was stirred at 40° C. for 18 h. After methanol was evaporated under reduced pressure, the mixture was diluted with water, acidified with 1 N HCl (160 mL) at 0° C., extracted with ethyl acetate (3×), dried over Na₂SO₄, and evaporated under reduced pressure to afford 15.00 g (96%) of (Z)-2-(2-((tert-butoxycarbonyl)amino)thiazol-4-yl)-2-(methoxyimino)acetic acid. ESI-MS m/z 302.0 (M+H)⁺.

### Step 6c

A mixture of (Z)-2-(2-((tert-butoxycarbonyl)amino)thiazol-4-yl)-2-(methoxyimino)acetic acid (15.00 g, 49.8 mmol), MeOH (275 mL), 1 N HCl (54 mL, 54 mmol), and 10% Pd/C (50% water-containing, 7.04 g, 3.3 mmol) was stirred under hydrogen balloon for 26 h. The mixture was filtered, washed with MeOH, concentrated to afford 16.36 g (100%) of 2-amino-2-(2-((tert-butoxycarbonyl)amino)thiazol-4-yl)acetic acid as HCl salt. ESI-MS m/z 274.0 (M+H)⁺.

### Step 6d

To a solution of the HCl salt (16.36 g, 52.8 mmol) of 2-amino-2-(2-((tert-butoxycarbonyl)amino)thiazol-4-yl)acetic acid, obtained as described above, water (300 mL), THF (300 mL), NaOH (4.12 g, 103 mmol), saturated NaHCO₃ (200 mL) at 0° C. was added 4-ethyl-2,3-dioxopiperazine-1-carbonyl chloride (14.35 g, 70 mmol) in THF. The resulting mixture was stirred at 0° C. for 2 h and then acidified with 1 N HCl (230 mL), extracted with ethyl acetate (3×), dried over Na₂SO₄, and evaporated under reduced pressure to afford 23.11 g (99%) of 2-(2-((tert-butoxycarbonyl)amino)thiazol-4-yl)-2-(4-ethyl-2,3-dioxopiperazine-1-carboxamido)acetic acid as a solid. ESI-MS m/z 883.2 (2M+H)⁺.

### Step 7. Synthesis of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2,2-diethoxyethyl)thio)benzoate

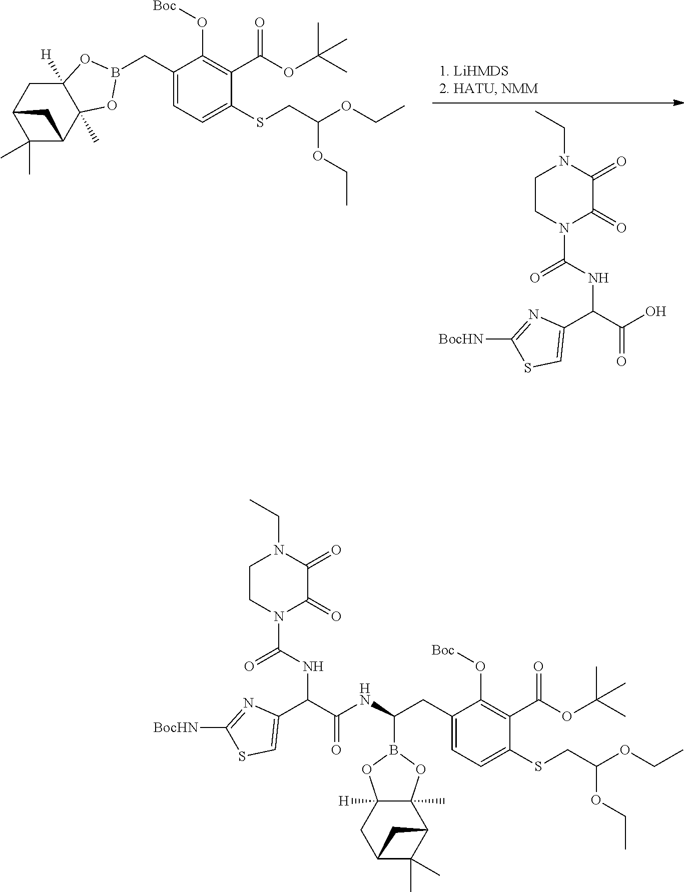 [SMILES: CCOC(CSc1ccc(CB2O[C@@H]3C[C@@H]4C[C@@H](C4(C)C)[C@]3(C)O2)c(OC(=O)OC(C)(C)C)c1C(=O)OC(C)(C)C)OCC ; "1. LiHMDS2. HATU, NMM" ; CCN1CCN(C(=O)NC(C(=O)O)c2csc(NC(=O)OC(C)(C)C)n2)C(=O)C1=O ; CCOC(CSc1ccc(C[C@H](NC(=O)C(NC(=O)N2CCN(CC)C(=O)C2=O)c2csc(NC(=O)OC(C)(C)C)n2)B2O[C@@H]3C[C@@H]4C[C@@H](C4(C)C)[C@]3(C)O2)c(OC(=O)OC(C)(C)C)c1C(=O)OC(C)(C)C)OCC]

To a solution of tert-butyl 2-((tert-butoxycarbonyl)oxy)-3-((S)-2-chloro-2-((3aS,4S,6S,7aR)-3a,5,5-trimethylhexahydro-4,6-methanobenzo[d][1,3,2]dioxaborol-2-yl)ethyl)-6-((2,2-diethoxyethyl)thio)benzoate (8.02 g, 11.7 mmol) in THF (100 mL) at −78° C. under argon was added LiHMDS (1.0 M in THF, 13 mL, 13 mmol) dropwise. The resulting mixture was allowed to slowly warm to room temperature overnight.

A mixture of 2-(2-((tert-butoxycarbonyl)amino)thiazol-4-yl)-2-(4-ethyl-2,3-dioxopiperazine-1-carboxamido)acetic acid (6.85 g, 15.5 mmol), prepared as described above, HATU (8.25 g, 21.7 mmol), NMM (3 mL, 27.3 mmol) in DMA (100 mL) was stirred at room temperature under argon for 1 h and then added to the reaction mixture above via cannula.

The resulting reaction mixture was stirred at room temperature for 2 d and then quenched with water, extracted with ethyl acetate (3×). The combined organic phase was dried over Na₂SO₄. After the solvent was evaporated under reduced pressure, the residue was purified by flash chromatography on silica gel (340 g column) eluted with 0 to 50% acetone/hexanes to afford 9.42 g (73.8%) of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2,2-diethoxyethyl)thio)benzoate as a solid. ESI-MS m/z 1041.4 (M-HOEt)⁺, 985.3 (M-HOEt-56)⁺.

### Step 8. Synthesis of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2-oxoethyl)thio)benzoate

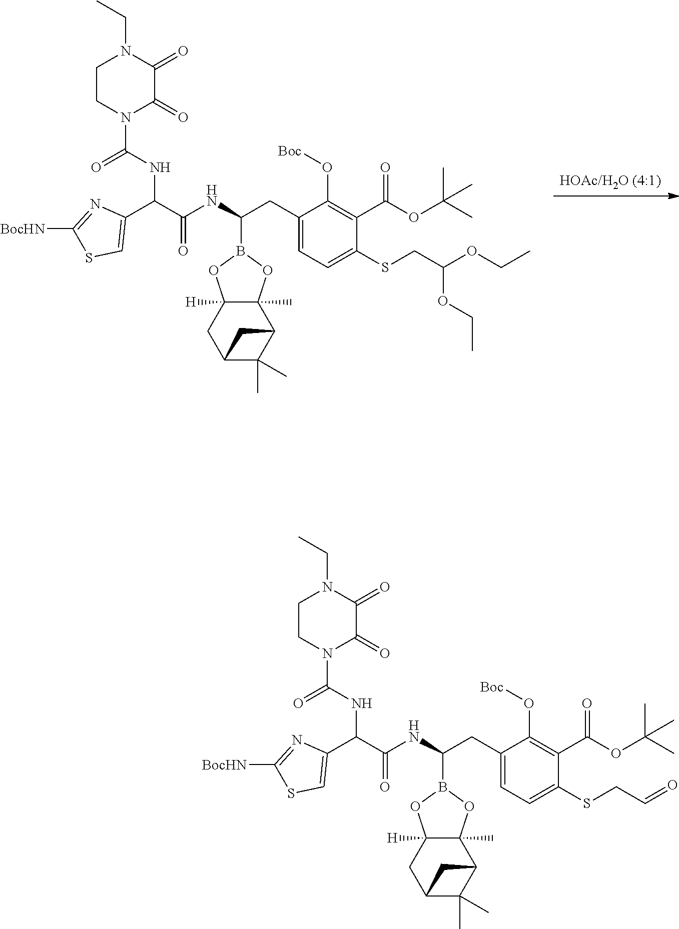 [SMILES: CCOC(CSc1ccc(C[C@H](NC(=O)C(NC(=O)N2CCN(CC)C(=O)C2=O)c2csc(NC(=O)OC(C)(C)C)n2)B2O[C@@H]3C[C@@H]4C[C@@H](C4(C)C)[C@]3(C)O2)c(OC(=O)OC(C)(C)C)c1C(=O)OC(C)(C)C)OCC ; "HOAc/H2O (4:1)" ; CCN1CCN(C(=O)NC(C(=O)N[C@@H](Cc2ccc(SCC=O)c(C(=O)OC(C)(C)C)c2OC(=O)OC(C)(C)C)B2O[C@@H]3C[C@@H]4C[C@@H](C4(C)C)[C@]3(C)O2)c2csc(NC(=O)OC(C)(C)C)n2)C(=O)C1=O]

A mixture of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2,2-diethoxyethyl)thio)benzoate (5.11 g, 4.70 mmol) in HOAc (90 mL) and water (30 mL) was vigorously stirred at room temperature for 5 d. The mixture was then lyophilized to give 4.61 g (96.8%) of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2-oxoethyl)thio)benzoate as a powder. ESI-MS m/z 1013.4 (M+H)⁺, 1035.3 (M+Na)⁺.

### Step 9. Synthesis of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2-(pyrrolidin-1-yl)ethyl)thio)benzoate

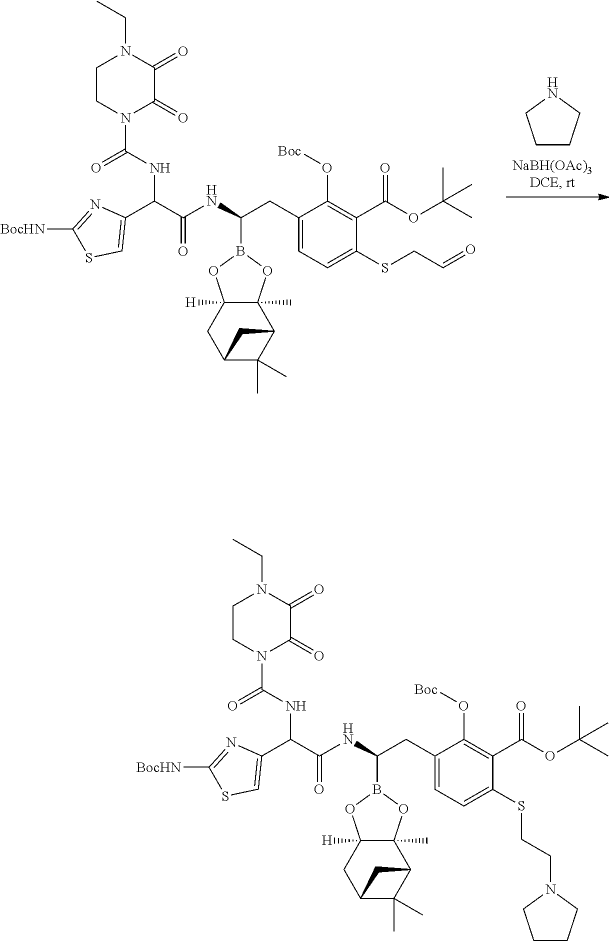

A mixture of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2-oxoethyl)thio)benzoate (1.66 g, 1.64 mmol), pyrrolidine (0.174 g, 2.45 mmol), and sodium triacetoxyborohydride (0.562 g, 2.65 mmol) in 1,2-dichloroethane (40 mL) was stirred at room temperature for 40 min. The mixture was then purified by flash chromatography on silica gel (50 g column) eluted with 0 to 20% MeOH/DCM to afford 0.458 g (26.2%) of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2-(pyrrolidin-1-yl)ethyl)thio)benzoate. ESI-MS m/z 1068.5 (M+H)⁺.

### Step 10. Synthesis of (3R)-3-(2-(2-aminothiazol-4-yl)-2-(4-ethyl-2,3-dioxopiperazine-1-carboxamido)acetamido)-2-hydroxy-7-((2-(pyrrolidin-1-yl)ethyl)thio)-3,4-dihydro-2H-benzo[e][1,2]oxaborinine-8-carboxylic acid

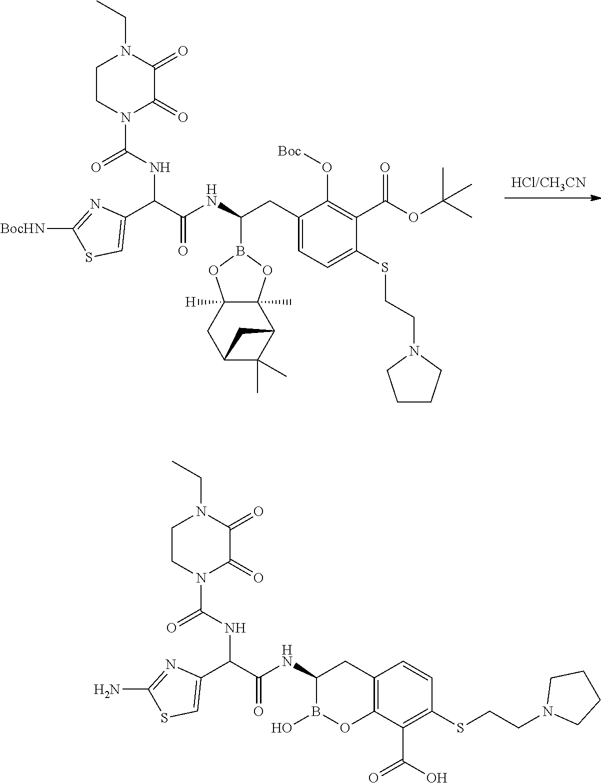 [SMILES: CCN1CCN(C(=O)NC(C(=O)N[C@@H](Cc2ccc(SCCN3CCCC3)c(C(=O)OC(C)(C)C)c2OC(=O)OC(C)(C)C)B2O[C@@H]3C[C@@H]4C[C@@H](C4(C)C)[C@]3(C)O2)c2csc(NC(=O)OC(C)(C)C)n2)C(=O)C1=O ; "HCl/CH3CN" ; CCN1CCN(C(=O)NC(C(=O)N[C@H]2Cc3ccc(SCCN4CCCC4)c(C(=O)O)c3OB2O)c2csc(N)n2)C(=O)C1=O]

A mixture of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2-(pyrrolidin-1-yl)ethyl)thio)benzoate (0.107 g), CH₃CN (9 mL), and 6 N HCl (3 mL) was stirred at room temperature for 16 h. The mixture was lyophilized, purified by reverse phase HPLC (Gilson, C18 column) and then lyophilized to give (3R)-3-(2-(2-aminothiazol-4-yl)-2-(4-ethyl-2,3-dioxopiperazine-1-carboxamido)acetamido)-2-hydroxy-7-((2-(pyrrolidin-1-yl)ethyl)thio)-3,4-dihydro-2H-benzo[e][1,2]oxaborinine-8-carboxylic acid as TFA salt. ESI-MS m/z 660.2 (M+H)⁺.

### Example 65: 1-(2-(((3R)-3-(2-(2-aminothiazol-4-yl)-2-(4-ethyl-2,3-dioxopiperazine-1-carboxamido)acetamido)-8-carboxy-2-hydroxy-3,4-dihydro-2H-benzo[e][1,2]oxaborinin-7-yl)thio)ethyl)-1-methylpyrrolidin-1-ium 2,2,2-trifluoroacetate

### Step 1. Synthesis of 1-(2-((2-(tert-butoxycarbonyl)-4-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-3-((tert-butoxycarbonyl)oxy)phenyl)thio)ethyl)-1-methylpyrrolidin-1-ium iodide

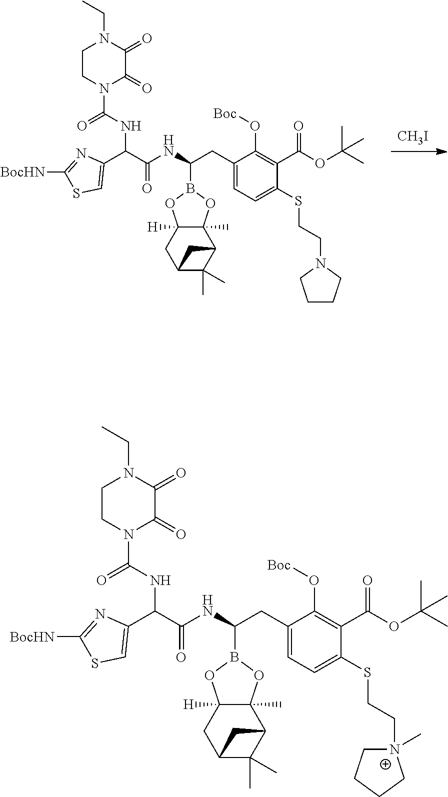

A mixture of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2-(pyrrolidin-1-yl)ethyl)thio)benzoate (0.229 g, 0.21 mmol) and Mel (0.1 mL, 1.6 mmol) in acetone (7 mL) was stirred at room temperature for 40 h. The mixture was then evaporated under reduced pressure to afford 1-(2-((2-(tert-butoxycarbonyl)-4-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-3-((tert-butoxycarbonyl)oxy)phenyl)thio)ethyl)-1-methylpyrrolidin-1-ium iodide, which was used in the next step directly. ESI-MS m/z 1082.5 (M⁺).

### Step 2. Synthesis of 1-(2-(((3R)-3-(2-(2-aminothiazol-4-yl)-2-(4-ethyl-2,3-dioxopiperazine-1-carboxamido)acetamido)-8-carboxy-2-hydroxy-3,4-dihydro-2H-benzo[e][1,2]oxaborinin-7-yl)thio)ethyl)-1-methylpyrrolidin-1-ium 2,2,2-trifluoroacetate

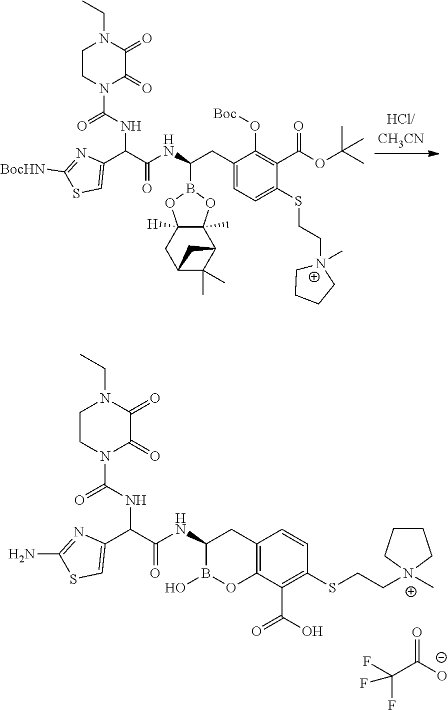 [SMILES: CCN1CCN(C(=O)NC(C(=O)N[C@@H](Cc2ccc(SCC[N+]3(C)CCCC3)c(C(=O)OC(C)(C)C)c2OC(=O)OC(C)(C)C)B2O[C@@H]3C[C@@H]4C[C@@H](C4(C)C)[C@]3(C)O2)c2csc(NC(=O)OC(C)(C)C)n2)C(=O)C1=O ; CCN1CCN(C(=O)NC(C(=O)N[C@H]2Cc3ccc(SCC[N+]4(C)CCCC4)c(C(=O)O)c3OB2O)c2csc(N)n2)C(=O)C1=O ; O=C([O-])C(F)(F)F]

A mixture of 1-(2-((2-(tert-butoxycarbonyl)-4-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-3-((tert-butoxycarbonyl)oxy)phenyl)thio)ethyl)-1-methylpyrrolidin-1-ium iodide, obtained as described above, CH₃CN (12 mL), and 6 N HCl (4 mL) was stirred at room temperature for 22 h. The mixture was lyophilized, purified by reverse phase HPLC (Gilson, C18 column) and then lyophilized to give 1-(2-(((3R)-3-(2-(2-aminothiazol-4-yl)-2-(4-ethyl-2,3-dioxopiperazine-1-carboxamido)acetamido)-8-carboxy-2-hydroxy-3,4-dihydro-2H-benzo[e][1,2]oxaborinin-7-yl)thio)ethyl)-1-methylpyrrolidin-1-ium as TFA salt. ESI-MS m/z 674.2 (M).

### Example 66: (3R)-7-((2-((2-aminoethyl)amino)ethyl)thio)-3-(2-(2-aminothiazol-4-yl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of tert-butyl 6-((2-((2-((tert-butoxycarbonyl)amino)ethyl)amino)ethyl)thio)-3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)benzoate

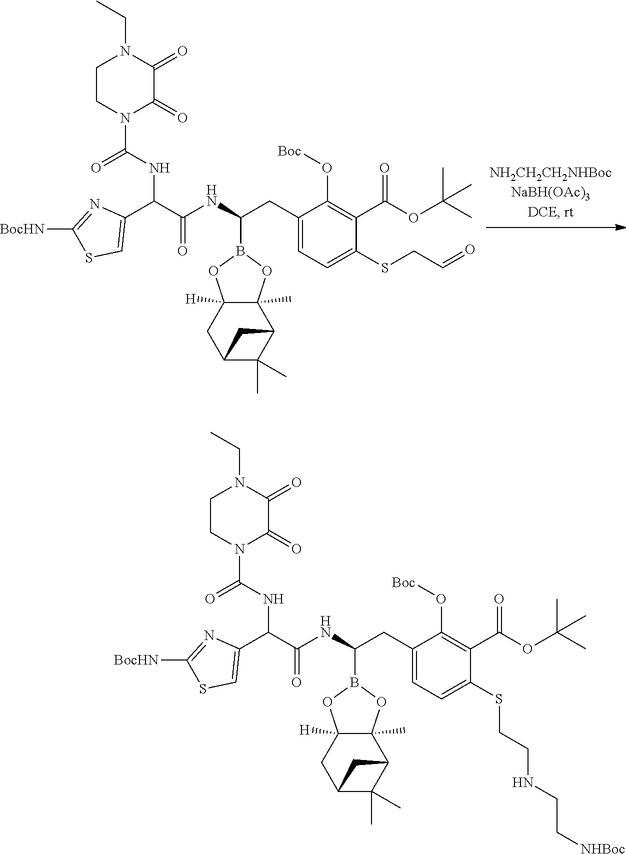 [SMILES: CCN1CCN(C(=O)NC(C(=O)N[C@@H](Cc2ccc(SCC=O)c(C(=O)OC(C)(C)C)c2OC(=O)OC(C)(C)C)B2O[C@@H]3C[C@@H]4C[C@@H](C4(C)C)[C@]3(C)O2)c2csc(NC(=O)OC(C)(C)C)n2)C(=O)C1=O ; CCN1CCN(C(=O)NC(C(=O)N[C@@H](Cc2ccc(SCCNCCNC(=O)OC(C)(C)C)c(C(=O)OC(C)(C)C)c2OC(=O)OC(C)(C)C)B2O[C@@H]3C[C@@H]4C[C@@H](C4(C)C)[C@]3(C)O2)c2csc(NC(=O)OC(C)(C)C)n2)C(=O)C1=O]

A mixture of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2-oxoethyl)thio)benzoate (0.588 g, 0.58 mmol), tert-butyl (2-aminoethyl)carbamate (0.144 g, 0.90 mmol), and sodium triacetoxyborohydride (0.222 g, 1.05 mmol) in 1,2-dichloroethane (17 mL) was stirred at room temperature for 30 min. The mixture was then purified by flash chromatography on silica gel (50 g column) eluted with 0 to 20% MeOH/DCM to afford 0.168 g (25%) of tert-butyl 6-((2-((2-((tert-butoxycarbonyl)amino)ethyl)amino)ethyl)thio)-3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)benzoate. ESI-MS m/z 1057.5 (M+H)⁺.

### Step 2. Synthesis of (3R)-7-((2-((2-aminoethyl)amino)ethyl)thio)-3-(2-(2-aminothiazol-4-yl)-2-(4-ethyl-2,3-dioxopiperazine-1-carboxamido)acetamido)-2-hydroxy-3,4-dihydro-2H-benzo[e][1,2]oxaborinine-8-carboxylic acid

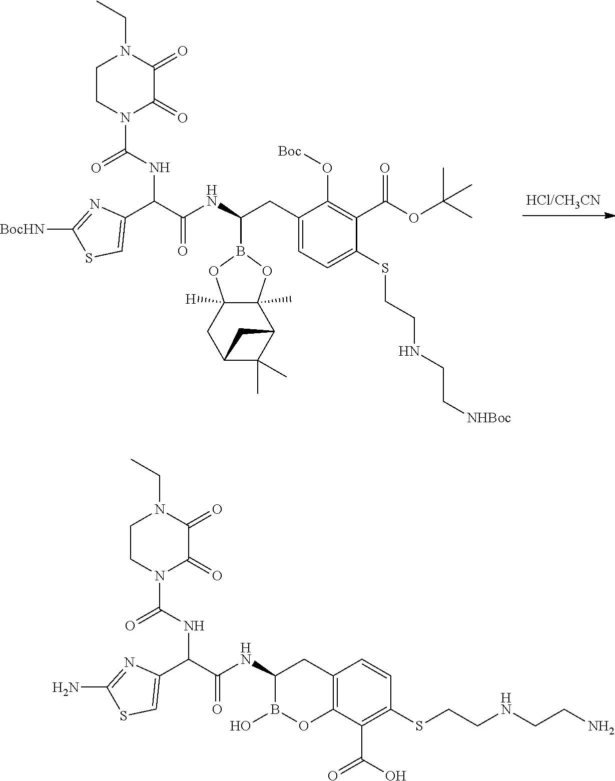 [SMILES: CCN1CCN(C(=O)NC(C(=O)N[C@@H](Cc2ccc(SCCNCCNC(=O)OC(C)(C)C)c(C(=O)OC(C)(C)C)c2OC(=O)OC(C)(C)C)B2O[C@@H]3C[C@@H]4C[C@@H](C4(C)C)[C@]3(C)O2)c2csc(NC(=O)OC(C)(C)C)n2)C(=O)C1=O ; CCN1CCN(C(=O)NC(C(=O)N[C@H]2Cc3ccc(SCCNCCN)c(C(=O)O)c3OB2O)c2csc(N)n2)C(=O)C1=O]

A mixture of tert-butyl 6-((2-((2-((tert-butoxycarbonyl)amino)ethyl)amino)ethyl)thio)-3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)benzoate (0.168 g), CH₃CN (12 mL), and 6 N HCl (4 mL) was stirred at room temperature for 22 h. The mixture was lyophilized, purified by reverse phase HPLC (Gilson, C18 column) and then lyophilized to give (3R)-7-((2-((2-aminoethyl)amino)ethyl)thio)-3-(2-(2-aminothiazol-4-yl)-2-(4-ethyl-2,3-dioxopiperazine-1-carboxamido)acetamido)-2-hydroxy-3,4-dihydro-2H-benzo[e][1,2]oxaborinine-8-carboxylic acid as TFA salt. ESI-MS m/z 649.2 (M+H)⁺.

### Example 67: (3R)-7-((2-aminoethyl)thio)-3-(2-(2-aminothiazol-4-yl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of tert-butyl 6-((2-aminoethyl)thio)-3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)benzoate

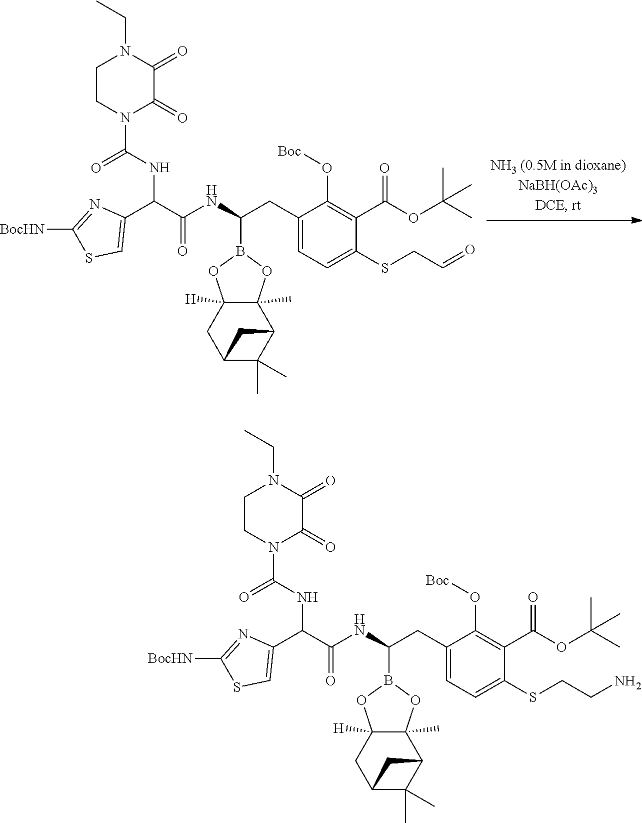

A mixture of tert-butyl 3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)-6-((2-oxoethyl)thio)benzoate (0.434 g, 0.43 mmol), ammonia (0.5 M in 1,4-dioxane, 2.0 mL, 1.0 mmol), and sodium triacetoxyborohydride (0.157 g, 0.74 mmol) in 1,2-dichloroethane (10 mL) was stirred at room temperature for 45 min. The mixture was then purified by flash chromatography on silica gel (50 g column) eluted with 0 to 20% MeOH/DCM to afford 0.209 g (48%) of tert-butyl 6-((2-aminoethyl)thio)-3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)benzoate. ESI-MS m/z 1015.4 (M+H)⁺.

### Step 2. Synthesis of (3R)-7-((2-aminoethyl)thio)-3-(2-(2-aminothiazol-4-yl)-2-(4-ethyl-2,3-dioxopiperazine-1-carboxamido)acetamido)-2-hydroxy-3,4-dihydro-2H-benzo[e][1,2]oxaborinine-8-carboxylic acid

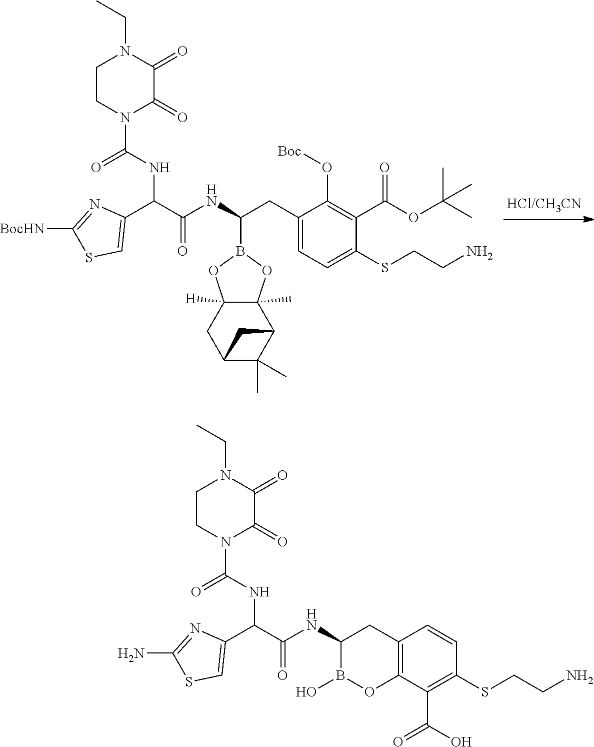 [SMILES: CCN1CCN(C(=O)NC(C(=O)N[C@@H](Cc2ccc(SCCN)c(C(=O)OC(C)(C)C)c2OC(=O)OC(C)(C)C)B2O[C@@H]3C[C@@H]4C[C@@H](C4(C)C)[C@]3(C)O2)c2csc(NC(=O)OC(C)(C)C)n2)C(=O)C1=O ; CCN1CCN(C(=O)NC(C(=O)N[C@H]2Cc3ccc(SCCN)c(C(=O)O)c3OB2O)c2csc(N)n2)C(=O)C1=O]

A mixture of tert-butyl 6-((2-aminoethyl)thio)-3-((2R)-2-(2-(2-((tert-butoxycarbonyl)amino)thiazol-4-yl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-((tert-butoxycarbonyl)oxy)benzoate (0.208 g), CH₃CN (12 mL), and 6 N HCl (4 mL) was stirred at room temperature for 24 h. The mixture was lyophilized, purified by reverse phase HPLC (Gilson, C18 column) and then lyophilized to give (3R)-7-((2-aminoethyl)thio)-3-(2-(2-aminothiazol-4-yl)-2-(4-ethyl-2,3-dioxopiperazine-1-carboxamido)acetamido)-2-hydroxy-3,4-dihydro-2H-benzo[e][1,2]oxaborinine-8-carboxylic acid as TFA salt. ESI-MS m/z 606.1 (M+H)⁺.

### Example 68: (3R)-3-(2-(4-ethyl-2,3-dioxopiperazine-1-carboxamido)-2-(3-hydroxyphenyl)acetamido)-2-hydroxy-3,4-dihydro-2H-benzo[e][1,2]oxaborinine-8-carboxylic acid

By following the procedures described in Step 8 of Example 15, utilizing 2-amino-2-(3-methoxyphenyl)acetic acid in place of 2-amino-2-(4-(tert-butoxycarbonyl)phenyl)acetic acid, and coupling the resulting acid with the chloride intermediate (prepared as previous reported, WO 2014/089365) by following the General method C, and then deprotection with BBr₃ as described in the General Method A, the title compound was prepared. ESI-MS m/z 525 (M+H)⁺.

### Example 69: (3R)-3-(2-(2,4-dihydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Example 70: (3R)-3-(2-(4-ethyl-2,3-dioxopiperazine-1-carboxamido)-2-(4-hydroxy-2-methoxyphenyl)acetamido)-2-hydroxy-3,4-dihydro-2H-benzo[e][1,2]oxaborinine-8-carboxylic acid

In a similar manner to the synthesis of Example 68, utilizing 2-amino-2-(2,4-dimethoxyphenyl)acetic acid in place of 2-amino-2-(3-methoxyphenyl)acetic acid, Example 69 and Example 70 were prepared after reversed phase HPLC purification. Example 70: ESI-MS m/z 541 (M+H)⁺. Example 71: ESI-MS m/z 555 (M+H)⁺.

### Example 71: (3R)-3-(2-(4-carboxy-3-fluorophenyl)-2-(2-chloro-6-fluoro-3,4-dihydroxybenzamido)acetamido)-7-fluoro-2-hydroxy-3,4-dihydro-2H-benzo[e][1,2]oxaborinine-8-carboxylic acid

The title compound was prepared in a similar manner to the synthesis of Example 7, utilizing 2-chloro-6-fluoro-3,4-dimethoxybenzoic acid in place of 2-chloro-3,4-dimethoxybenzoic acid. ESI-MS m/z 609 (MH)⁺.

### Example 72: (3R)-3-(2-(3-aminophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

In a similar manner to the synthesis of Example 17, utilizing 2-((tert-butoxycarbonyl)amino)-2-(3-nitrophenyl)acetic acid in place of 2-((tert-butoxycarbonyl)amino)-2-(4-methoxy-3-nitrophenyl)acetic acid in Step 2, and the hydrogenation product in Step 5 was treated with excess BBr₃ at RT for 3 h, affording the title compound after reversed phase HPLC purification. ESI-MS m/z 524 (M+1)⁺.

### Example 73: (3R)-3-(2-(3-(2-chloro-3,4-dihydroxybenzamido)phenyl)-2-(4-ethyl-2,3-dioxopiperazine-1-carboxamido)acetamido)-2-hydroxy-3,4-dihydro-2H-benzo[e][1,2]oxaborinine-8-carboxylic acid

In a similar manner to the synthesis of Example 17, utilizing 2-((tert-butoxycarbonyl)amino)-2-(3-nitrophenyl)acetic acid in place of 2-((tert-butoxycarbonyl)amino)-2-(4-methoxy-3-nitrophenyl)acetic acid in Step 2, and the hydrogenation product in Step 5 was reacted with 2-chloro-3,4-dimethoxybenzoyl chloride, then the resulting product was treated with excess BBr₃ at RT overnight, affording the title compound after reversed phase HPLC purification. ESI-MS m/z 694/696 (MH/MH+2)⁺.

### Example 74: (3R)-3-(2-(3-(2-chloro-3,4-dihydroxybenzamido)-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

In a similar manner to the synthesis of Example 17, the hydrogenation product in Step 5 was reacted with 2-chloro-3,4-dimethoxybenzoyl chloride, then the resulting product was treated with excess BBr₃ at RT overnight, affording the title compound after reversed phase HPLC purification. ESI-MS m/z 710/712 (MH/MH+2)⁺.

### Example 75: (3R)-3-(2-(4-ethyl-2,3-dioxopiperazine-1-carboxamido)-2-(3-nitrophenyl)acetamido)-2-hydroxy-3,4-dihydro-2H-benzo[e][1,2]oxaborinine-8-carboxylic acid

In a similar manner to the synthesis of Example 72, skipping the hydrogenation step, the title compound was obtained after reversed phase HPLC purification. ESI-MS m/z 554 (M+1)⁺.

### Example 76: (3R)-3-(2-(4-ethyl-2,3-dioxopiperazine-1-carboxamido)-2-(3-(methylsulfonamido)phenyl)acetamido)-2-hydroxy-3,4-dihydro-2H-benzo[e][1,2]oxaborinine-8-carboxylic acid

In a similar manner to the synthesis of Example 73, the hydrogenation product was reacted with methanesulfonyl chloride in the presence of pyridine, and the resulting crude product was treated with excess BBr₃ at RT for 3 h, affording the title compound after reversed phase HPLC purification. ESI-MS m/z 602 (M+1)⁺.

### Example 77: (3R)-3-(2-(2,5-dihydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

In a similar manner to the synthesis of Example 68, utilizing 2-amino-2-(2,5-dimethoxyphenyl)acetic acid in place of 2-amino-2-(3-methoxyphenyl)acetic acid, the title compound was prepared after reversed phase HPLC purification. ESI-MS m/z 541 (M+H)⁺. Example 70: ESI-MS m/z 541 (M+H)⁺.

### Example 78: (3R)-3-(2-(4-carboxy-3-fluorophenyl)-2-(4-(2-guanidinoethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 51, utilizing 2-(4-(tert-butoxycarbonyl)-3-fluorophenyl)-2-((tert-butoxycarbonyl)amino)acetic acid in place of 2-(5-(benzyloxy)-3-fluoropyridin-2-yl)-2-((tert-butoxycarbonyl)amino)acetic acid. ESI-MS m/z 646 (MH)⁺.

### Example 79: (3R)-3-(2-(3,5-dihydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

In a similar manner to the synthesis of Example 17, utilizing 3,5-dimethoxybenzaldehyde in place of 4-methoxy-3-nitrobenzaldehyde in Step 1, Example 79 was obtained as a mixture of two diastereomers after reversed phase HPLC purification. ESI-MS m/z 541 (M+1)⁺.

### Example 80: (R)-3-((R)-2-(4-(2-(2-chloro-6-fluoro-3,4-dihydroxybenzamido)ethyl)-2,3-dioxopiperazine-1-carboxamido)-3,3-dimethylbutanamido)-7-fluoro-2-hydroxy-3,4-dihydro-2H-benzo[e][1,2]oxaborinine-8-carboxylic acid

The title compound was prepared in a similar manner to the synthesis of Example 5, utilizing (R)-2-((tert-butoxycarbonyl)amino)-3,3-dimethylbutanoic acid in place of 2-(5-(benzyloxy)-3-fluoropyridin-2-yl)-2-((tert-butoxycarbonyl)amino)acetic acid. ESI-MS m/z 710 (MH)⁺.

### Example 81: (R)-3-((S)-2-(3,5-dihydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Example 82: (R)-3-((R)-2-(3,5-dihydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

Example 79 was purified by reversed phase HPLC, Example 81 was isolated as the first eluting peak, Example 82 was isolated as the second eluting peak. ESI-MS m/z 541 (M+1)⁺.

### Example 83: (R)-3-((S)-2-(4-(2-aminoethyl)-2,3-dioxopiperazine-1-carboxamido)-2-(4-carboxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(4-(tert-butoxycarbonyl)phenyl)acetic acid

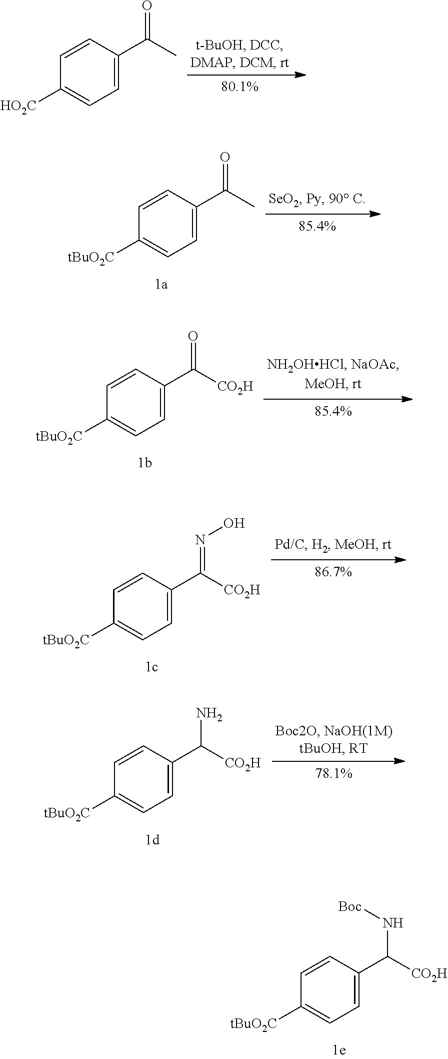 [SMILES: CC(=O)c1ccc(C(=O)O)cc1 ; CC(=O)c1ccc(C(=O)OC(C)(C)C)cc1 ; CC(C)(C)OC(=O)c1ccc(C(=O)C(=O)O)cc1 ; CC(C)(C)OC(=O)c1ccc(/C(=N/O)C(=O)O)cc1 ; CC(C)(C)OC(=O)c1ccc(C(N)C(=O)O)cc1 ; CC(C)(C)OC(=O)NC(C(=O)O)c1ccc(C(=O)OC(C)(C)C)cc1]

### Step 1a. Synthesis of tert-butyl 4-acetylbenzoate

To a mixture of 4-acetylbenzoic acid (30 g, 183 mmol) in DCM (300 mL) was added DCC (45 g, 219 mmol), DMAP (22.3 g, 183 mmol) and tBuOH (20 g, 274 mmol) at room temperature. The reaction mixture was stirred at room temperature for 16 h. After the solvent was evaporated under reduced pressure, the residue was purified by flash chromatography on silica gel (300 g column) eluted with 0 to 25% ethyl acetate/hexanes to afford 32 g (80.1%) of tert-butyl 4-acetylbenzoate. ESI-MS m/z 221.1 (M+H)⁺.

### Step 1b. Synthesis of 2-(4-(tert-butoxycarbonyl)phenyl)-2-oxoacetic acid

A solution of tert-butyl 4-acetylbenzoate (32 g, 144 mmol) in 400 mL pyridine was treated with SeO₂ (32 g, 288 mmol), and the resulting mixture was stirred for 6 h at 90° C. The mixture was filtered, the filtrate was concentrated in vacuo, and the residue was acidified to pH˜2 with 1 mol/L HCl(aq), extracted with EA, dried over Na₂SO₄, concentrated under vacuum to give the title compound, 29 g (80.2%). ESI-MS m/z 251.0 (M+H)⁺.

### Step 1c. Synthesis of (Z)-2-(4-(tert-butoxycarbonyl)phenyl)-2-(hydroxyimino)acetic acid

A solution of the product from Step 1b (29 g, 116 mmol) in 300 mL MeOH was treated with NaOAc (10.5 g, 127 mmol) and NH₂.HCl (8.8 g, 127 mmol). The reaction was stirred for 12 h at room temperature. The mixture was concentrated in vacuo and the residue was diluted with water, extracted with EA, dried over Na₂SO₄, concentrated under vacuum to afford the title compound, 30 g (97.6%). ESI-MS m/z 266.1 (M+H)⁺.

### Step 1d. Synthesis of 2-amino-2-(4-(tert-butoxycarbonyl)phenyl)acetic acid

A solution of the product from Step 1c (30 g, 113 mmol) in 500 mL MeOH was treated with Pd/C (15 g) for 15 h at room temperature in the presence of hydrogen. The mixture was filtered. The filtrate was concentrated in vacuo to provide the title compound (25 g, 88%). ESI-MS m/z 252.1 (M+H)⁺.

### Step 1e. Synthesis of 2-((tert-butoxycarbonyl)amino)-2-(4-(tert-butoxycarbonyl)phenyl)acetic acid

A solution of the product from Step id (25 g, 99 mmol) in 240 mL 1 mol/L NaOH (aq) was treated with a solution of Boc₂O (23.7 g, 109 mmol) in 180 ml tBuOH at 0° C., and the resulting mixture was stirred at room temperature for 18 h. The mixture was concentrated under vacuum, then the remaining solution was adjusted to pH-3 with 1 mol/L HCl (aq). The resulting mixture was extracted with DCM, dried (Na₂SO₄), concentrated in vacuo, and the residue purified by silica gel chromatography (EA-PE, 0-35%) to provide the title compound (26 g, 74.5%). ESI-MS m/z 352.1 (M+H)⁺.

### Step 2: Synthesis of tert-butyl 3-((2R)-2-(2-((tert-butoxycarbonyl)amino)-2-(4-(tert-butoxycarbonyl)phenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

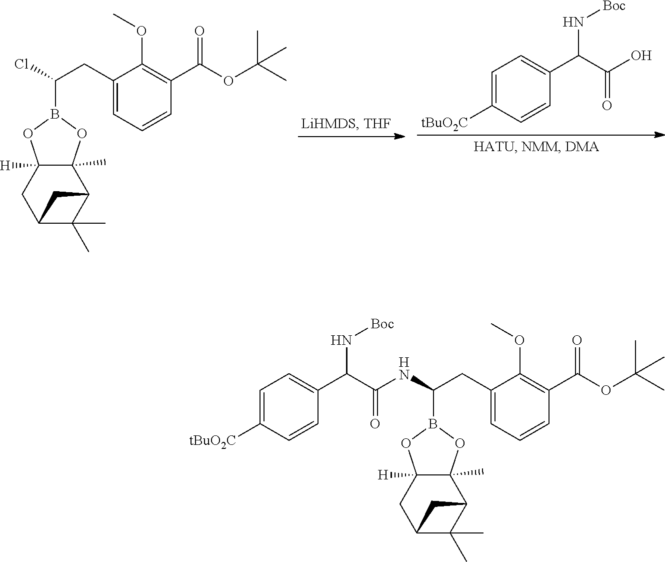 [SMILES: COc1c(C[C@@H](Cl)B2O[C@@H]3C[C@@H]4C[C@@H](C4(C)C)[C@]3(C)O2)cccc1C(=O)OC(C)(C)C ; CC(C)(C)OC(=O)NC(C(=O)O)c1ccc(C(=O)OC(C)(C)C)cc1 ; COc1c(C[C@H](NC(=O)C(NC(=O)OC(C)(C)C)c2ccc(C(=O)OC(C)(C)C)cc2)B2O[C@@H]3C[C@@H]4C[C@@H](C4(C)C)[C@]3(C)O2)cccc1C(=O)OC(C)(C)C]

By following the General Procedure C, the chloride (prepared as previous reported, WO 2014/089365) was treated with LiHMDS, and then coupled 2-((tert-butoxycarbonyl)amino)-2-(4-(tert-butoxycarbonyl)phenyl)acetic acid in the presence of HATU and NMM, yielding the title compound. ESI-MS m/z 763.4 (M+H)⁺.

### Step 3: Synthesis of (R)-3-((S)-2-(4-(2-aminoethyl)-2,3-dioxopiperazine-1-carboxamido)-2-(4-carboxyphenyl)acetamido)-2-hydroxy-3,4-dihydro-2H-benzo[e][1,2]oxaborinine-8-carboxylic acid

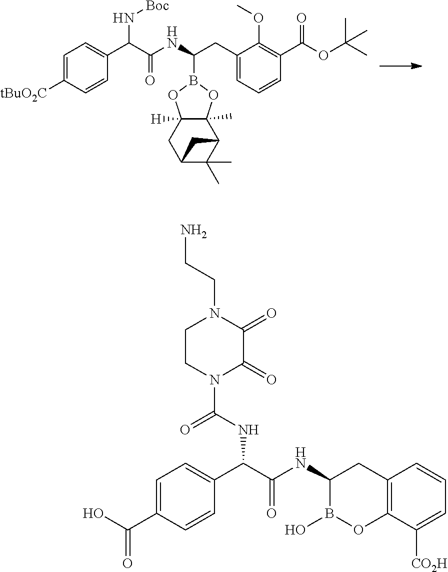 [SMILES: COc1c(C[C@H](NC(=O)C(NC(=O)OC(C)(C)C)c2ccc(C(=O)OC(C)(C)C)cc2)B2O[C@@H]3C[C@@H]4C[C@@H](C4(C)C)[C@]3(C)O2)cccc1C(=O)OC(C)(C)C ; NCCN1CCN(C(=O)N[C@H](C(=O)N[C@H]2Cc3cccc(C(=O)O)c3OB2O)c2ccc(C(=O)O)cc2)C(=O)C1=O]

The title compound was prepared in a similar manner to the synthesis of Example 1, utilizing tert-butyl (2-(4-(chlorocarbonyl)-2,3-dioxopiperazin-1-yl)ethyl)carbamate in place of tert-butyl (3-(4-(chlorocarbonyl)-2,3-dioxopiperazin-1-yl)propyl)carbamate, isolated as the second eluting peak. ESI-MS m/z 568.2 (M+H)⁺.

### Example 84: (R)-3-((R)-2-(4-(2-aminoethyl)-2,3-dioxopiperazine-1-carboxamido)-2-(4-carboxyphenyl)acetamido)-2-hydroxy-3,4-dihydro-2H-benzo[e][1,2]oxaborinine-8-carboxylic acid

In a same manner to the synthesis of Example 83, the title compound was prepared after reversed phase HPLC purification, isolated as the first eluting peak. ESI-MS m/z 568.3 (M+H)⁺.

### Example 85: (R)-3-((S)-2-(4-(2-aminoethyl)-2,3-dioxopiperazine-1-carboxamido)-2-(2-chloro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2-chloro-4-methoxyphenyl)acetic acid

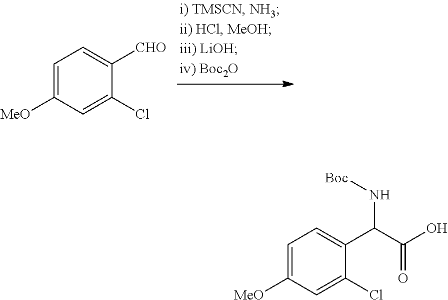 [SMILES: COc1ccc(C=O)c(Cl)c1 ; COc1ccc(C(NC(=O)OC(C)(C)C)C(=O)O)c(Cl)c1]

By following the same procedures as described for the synthesis of Synthesis of 2-((tert-butoxycarbonyl)amino)-2-(4-methoxy-3-nitrophenyl)acetic acid in Step 1 of Example 17, the title compound was prepared from 2-chloro-4-methoxybenzaldehyde.

### Step 2. Synthesis of tert-butyl 3-((2R)-2-(2-((tert-butoxycarbonyl)amino)-2-(2-chloro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

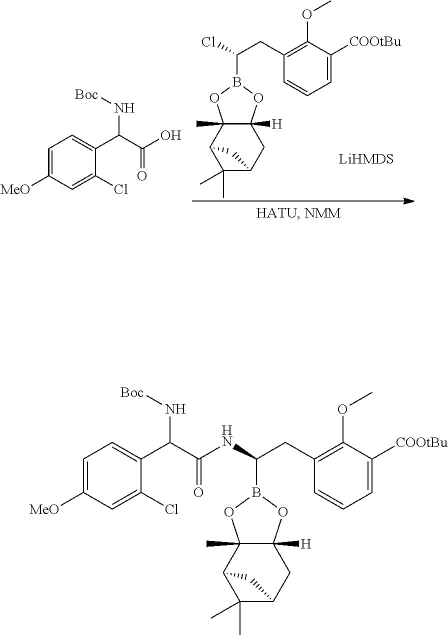

By following the General coupling method C, the title compound was prepared from the above acid and the chloride intermediate (prepared as previous reported, WO 2014/089365). ESI-MS m/z 728 (M+H)⁺.

### Step 3. Synthesis of tert-butyl (2-(4-(chlorocarbonyl)-2,3-dioxopiperazin-1-yl)ethyl)carbamate

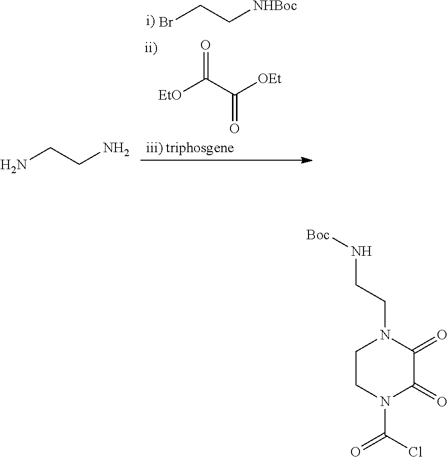

By following the same procedures as described for the synthesis of tert-butyl (2-(4-(chlorocarbonyl)-2,3-dioxopiperazin-1-yl)ethyl)carbamate in Step 1 of Example 26.

### Step 4. Synthesis of (R)-3-((S)-2-(4-(2-aminoethyl)-2,3-dioxopiperazine-1-carboxamido)-2-(2-chloro-4-hydroxyphenyl)acetamido)-2-hydroxy-3,4-dihydro-2H-benzo[e][1,2]oxaborinine-8-carboxylic acid

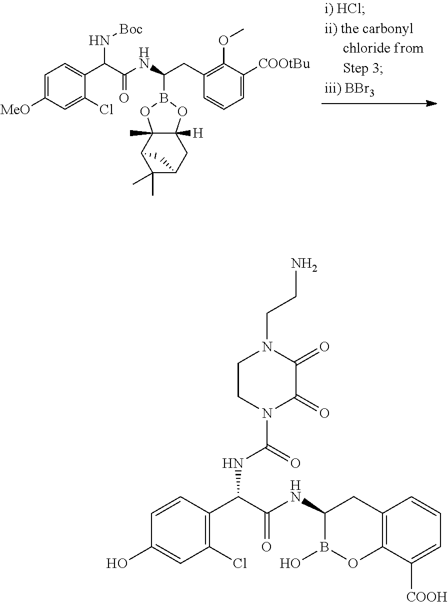

In a similar manner to the synthesis of Example 26, the title compound was prepared after reversed phase HPLC purification, isolated as the first eluting peak. ESI-MS m/z 574/576 (MH/MH+2)⁺.

### Example 86: (R)-3-((R)-2-(4-(2-aminoethyl)-2,3-dioxopiperazine-1-carboxamido)-2-(2-chloro-4-hydroxyphenyl)acetamido)-2-hydroxy-3,4-dihydro-2H-benzo[e][1,2]oxaborinine-8-carboxylic acid

In a same manner to the synthesis of Example 86, the title compound was prepared after reversed phase HPLC purification, isolated as the second eluting peak. ESI-MS m/z 574/576 (MH/MH+2)⁺.

### Example 87: (3R)-3-(2-(4-(2-aminoethyl)-2,3-dioxopiperazine-1-carboxamido)-2-(5-chloro-2-fluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(5-chloro-2-fluoro-4-methoxyphenyl)acetic acid

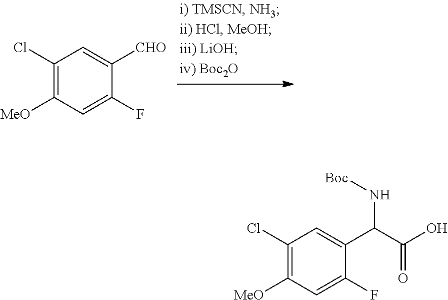 [SMILES: COc1cc(F)c(C=O)cc1Cl ; COc1cc(F)c(C(NC(=O)OC(C)(C)C)C(=O)O)cc1Cl]

By following the same procedures as described for the synthesis of Synthesis of 2-((tert-butoxycarbonyl)amino)-2-(4-methoxy-3-nitrophenyl)acetic acid in Step 1 of Example 17, the title compound was prepared from 5-chloro-2-fluoro-4-methoxybenzaldehyde.

### Step 2. Synthesis of tert-butyl 3-((2R)-2-(2-((tert-butoxycarbonyl)amino)-2-(5-chloro-2-fluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

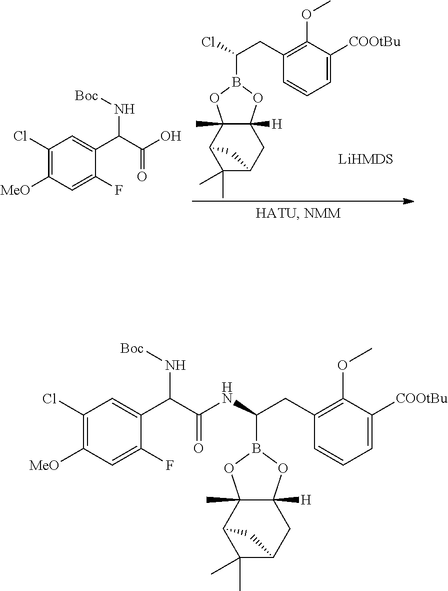

By following the General coupling method C, the title compound was prepared from the above acid and the chloride intermediate (prepared as previous reported, WO 2014/089365). ESI-MS m/z 746 (M+H).

### Step 3. Synthesis of tert-butyl (2-(4-(chlorocarbonyl)-2,3-dioxopiperazin-1-yl)ethyl)carbamate

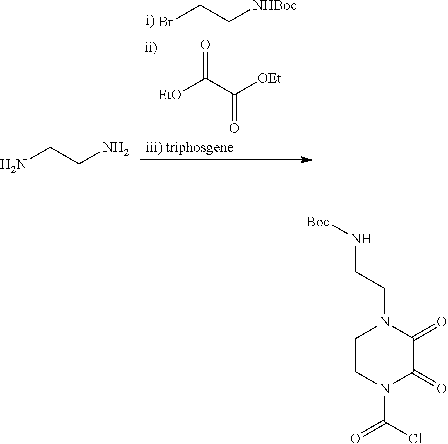

By following the same procedures as described for the synthesis of tert-butyl (2-(4-(chlorocarbonyl)-2,3-dioxopiperazin-1-yl)ethyl)carbamate in Step 1 of Example 26.

### Step 4. Synthesis of (3R)-3-(2-(4-(2-aminoethyl)-2,3-dioxopiperazine-1-carboxamido)-2-(5-chloro-2-fluoro-4-hydroxyphenyl)acetamido)-2-hydroxy-3,4-dihydro-2H-benzo[e][1,2]oxaborinine-8-carboxylic acid

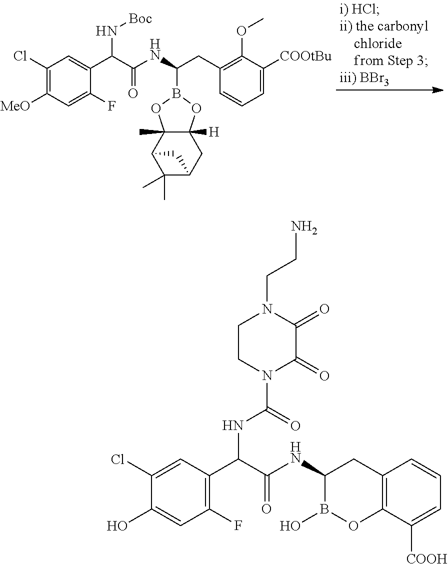

In a similar manner to the synthesis of Example 26, the title compound was prepared after reversed phase HPLC purification. ESI-MS m/z 592/594 (MH/MH+2)⁺.

### Example 88: (R)-3-((S)-2-(4-(2-aminoethyl)-2,3-dioxopiperazine-1-carboxamido)-2-(2-fluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2-fluoro-4-methoxyphenyl)acetic acid

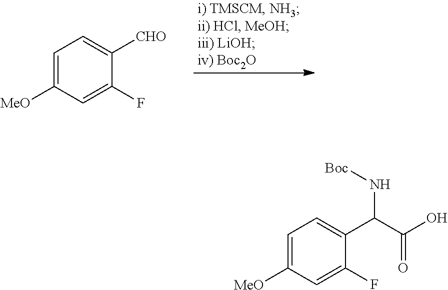 [SMILES: COc1ccc(C=O)c(F)c1 ; COc1ccc(C(NC(=O)OC(C)(C)C)C(=O)O)c(F)c1]

By following the same procedures as described for the synthesis of Synthesis of 2-((tert-butoxycarbonyl)amino)-2-(4-methoxy-3-nitrophenyl)acetic acid in Step 1 of Example 17, the title compound was prepared from 2-fluoro-4-methoxybenzaldehyde.

### Step 2. Synthesis of tert-butyl 3-((2R)-2-(2-((tert-butoxycarbonyl)amino)-2-(2-fluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

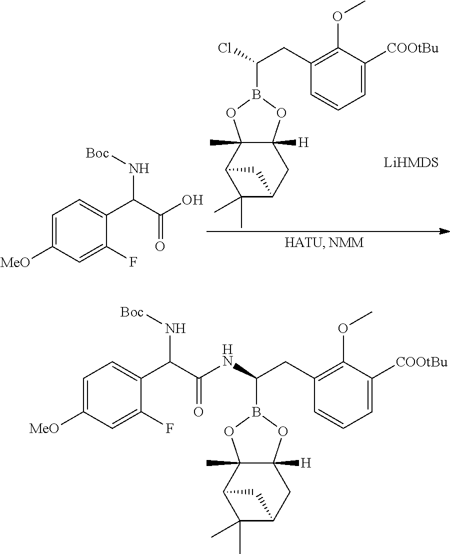

By following the General coupling method C, the title compound was prepared from the above acid and the chloride intermediate (prepared as previous reported, WO 2014/089365). ESI-MS m/z 711 (M+H)⁺.

### Step 3. Synthesis of tert-butyl (2-(4-(chlorocarbonyl)-2,3-dioxopiperazin-1-yl)ethyl)carbamate

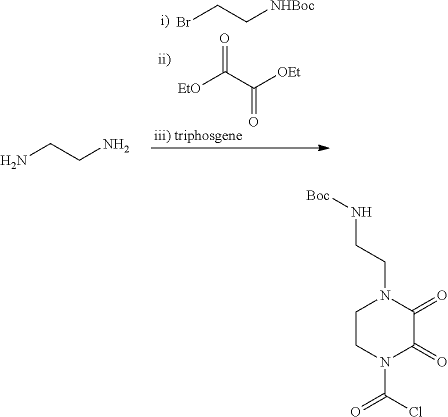

By following the same procedures as described for the synthesis of tert-butyl (2-(4-(chlorocarbonyl)-2,3-dioxopiperazin-1-yl)ethyl)carbamate in Step 1 of Example 26.

### Step 4. Synthesis of (R)-3-((S)-2-(4-(2-aminoethyl)-2,3-dioxopiperazine-1-carboxamido)-2-(2-fluoro-4-hydroxyphenyl)acetamido)-2-hydroxy-3,4-dihydro-2H-benzo[e][1,2]oxaborinine-8-carboxylic acid

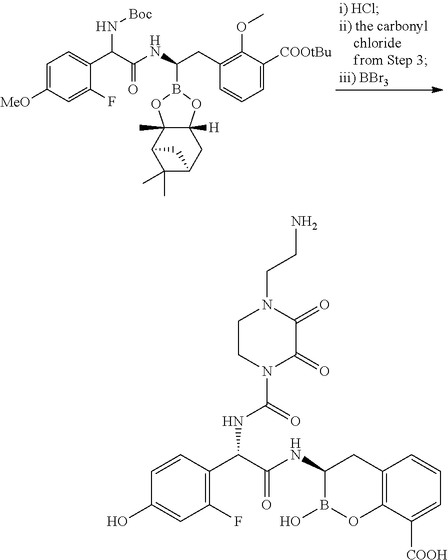

In a similar manner to the synthesis of Example 26, the title compound was prepared after reversed phase HPLC purification. isolated as the second eluting peak. ESI-MS m/z 557/559 (MH/MH+2)⁺.

### Example 89: (R)-3-((R)-2-(4-(2-aminoethyl)-2,3-dioxopiperazine-1-carboxamido)-2-(2-fluoro-4-hydroxyphenyl)acetamido)-2-hydroxy-3,4-dihydro-2H-benzo[e][1,2]oxaborinine-8-carboxylic acid

In a same manner to the synthesis of Example 88, the title compound was prepared after reversed phase HPLC purification, isolated as the first eluting peak. ESI-MS m/z 557/559 (MH/MH+2)⁺.

### Example 90: (R)-3-((R)-2-(4-(2-aminoethyl)-2,3-dioxopiperazine-1-carboxamido)-2-(2,5-dichloro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2,5-dichloro-4-methoxyphenyl)acetic acid in place of 2-(4-(tert-butoxycarbonyl)-2,6-difluorophenyl)-2-((tert-butoxycarbonyl)amino)acetic acid in step 6, ESI-MS m/z 608 (MH)⁺.

### Example 91: (R)-3-((R)-2-(2,5-dichloro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2,5-dichloro-4-methoxyphenyl)acetic acid in place of 2-(4-(tert-butoxycarbonyl)-2,6-difluorophenyl)-2-((tert-butoxycarbonyl)amino)acetic acid in step 6, isolated as the second eluting peak. ESI-MS m/z 593 (MH)⁺.

### Example 92: ((R)-3-((S)-2-(2,5-dichloro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 91, the title compound was prepared after reversed phase HPLC purification, isolated as the first eluting peak. ESI-MS m/z 593 (MH)⁺.

### Example 93: (3R)-3-(2-(4-(2-aminoethyl)-2,3-dioxopiperazine-1-carboxamido)-2-(2,5-difluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2,5-difluoro-4-methoxyphenyl)acetic acid in place of 2-(4-(tert-butoxycarbonyl)-2,6-difluorophenyl)-2-((tert-butoxycarbonyl)amino)acetic acid ESI-MS m/z 576 (MH)⁺.

### Example 94: (3R)-3-(2-(4-(2-aminoethyl)-2,3-dioxopiperazine-1-carboxamido)-2-(2-chloro-6-fluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2-chloro-6-fluoro-4-methoxyphenyl)acetic acid in place of 2-(4-(tert-butoxycarbonyl)-2,6-difluorophenyl)-2-((tert-butoxycarbonyl)amino)acetic acid. ESI-MS m/z 592 (MH)⁺.

### Example 95: (3R)-3-(2-(2-chloro-6-fluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2-chloro-6-fluoro-4-methoxyphenyl)acetic acid in place of 2-(4-(tert-butoxycarbonyl)-2,6-difluorophenyl)-2-((tert-butoxycarbonyl)amino)acetic acid. ESI-MS m/z 577 (MH)⁺.

### Example 96: (3R)-3-(2-(4-ethyl-2,3-dioxopiperazine-1-carboxamido)-2-(3-(pyrimidine-5-carboxamido)phenyl)acetamido)-2-hydroxy-3,4-dihydro-2H-benzo[e][1,2]oxaborinine-8-carboxylic acid

In a similar manner to the synthesis of Example 73, utilizing pyrimidine-5-carbonyl chloride in place of 2-chloro-3,4-dimethoxybenzoyl chloride, the title compound was prepared after reversed phase HPLC purification. ESI-MS m/z 630 (M+1)⁺.

### Example 97: (3R)-3-(2-(4-ethyl-2,3-dioxopiperazine-1-carboxamido)-2-(3-(nicotinamido)phenyl)acetamido)-2-hydroxy-3,4-dihydro-2H-benzo[e][1,2]oxaborinine-8-carboxylic acid

In a similar manner to the synthesis of Example 73, utilizing nicotinoyl chloride in place of 2-chloro-3,4-dimethoxybenzoyl chloride, the title compound was prepared after reversed phase HPLC purification. ESI-MS m/z 629 (M+1)⁺.

### Example 98: (3R)-3-(2-(4-ethyl-2,3-dioxopiperazine-1-carboxamido)-2-(3-(sulfamoylamino)phenyl)acetamido)-2-hydroxy-3,4-dihydro-2H-benzo[e][1,2]oxaborinine-8-carboxylic acid

In a similar manner to the synthesis of Example 73, utilizing tert-butyl (chlorosulfonyl)carbamate in place of 2-chloro-3,4-dimethoxybenzoyl chloride, the title compound was prepared after reversed phase HPLC purification. ESI-MS m/z 603 (M+1)⁺.

### Example 99: (3R)-3-(2-(4-ethyl-2,3-dioxopiperazine-1-carboxamido)-2-(3-sulfamoylphenyl)acetamido)-2-hydroxy-3,4-dihydro-2H-benzo[e][1,2]oxaborinine-8-carboxylic acid

In a similar manner to the synthesis of Example 17, utilizing 3-formylbenzenesulfonamide in place of 4-methoxy-3-nitrobenzaldehyde in Step 1, the title compound was prepared after reversed phase HPLC purification. ESI-MS m/z 588 (M+1)⁺.

### Example 100: (3R)-3-(2-(4-(2-fluoroethyl)-2,3-dioxopiperazine-1-carboxamido)-2-(3-sulfamoylphenyl)acetamido)-2-hydroxy-3,4-dihydro-2H-benzo[e][1,2]oxaborinine-8-carboxylic acid

In a similar manner to the synthesis of Example 99, utilizing 4-(2-fluoroethyl)-2,3-dioxopiperazine-1-carbonyl chloride in place of 4-ethyl-2,3-dioxopiperazine-1-carbonyl chloride for the acylation in Step 4, the title compound was prepared after reversed phase HPLC purification. ESI-MS m/z 606 (M+1)⁺.

### Example 101: (3R)-3-(2-(5-chloro-2-fluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(5-chloro-2-fluoro-4-methoxyphenyl)acetic acid

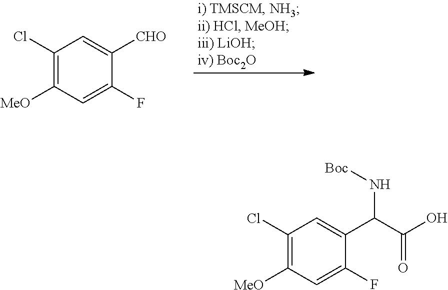 [SMILES: COc1cc(F)c(C=O)cc1Cl ; COc1cc(F)c(C(NC(=O)OC(C)(C)C)C(=O)O)cc1Cl]

By following the same procedures as described for the synthesis of Synthesis of 2-((tert-butoxycarbonyl)amino)-2-(4-methoxy-3-nitrophenyl)acetic acid in Step 1 of Example 17, the title compound was prepared from 5-chloro-2-fluoro-4-methoxybenzaldehyde.

### Step 2. Synthesis of tert-butyl 3-((2R)-2-(2-((tert-butoxycarbonyl)amino)-2-(5-chloro-2-fluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

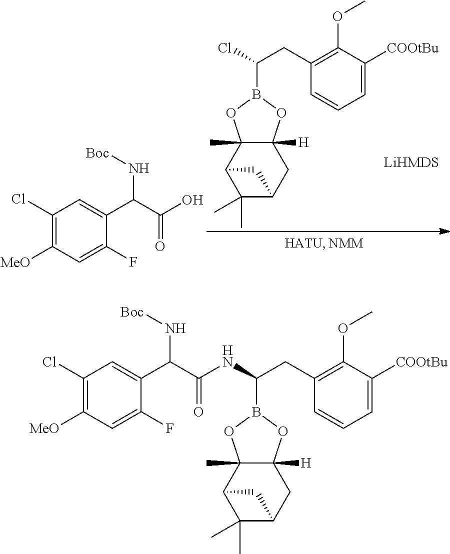

By following the General coupling method C, the title compound was prepared from the above acid and the chloride intermediate (prepared as previous reported, WO 2014/089365). ESI-MS m/z 746 (M+H)⁺.

### Step 3. Synthesis of (3R)-3-(2-(5-chloro-2-fluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

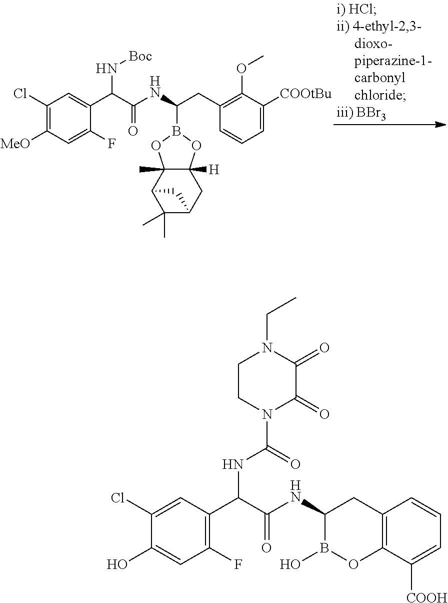

In a similar manner to the synthesis of Example 26, the title compound was prepared after reversed phase HPLC purification. ESI-MS m/z 576/578 (MH/MH+2)⁺.

### Example 102: (R)-3-((2R,3R)-2-(4-(2-(2-chloro-6-fluoro-3,4-dihydroxybenzamido)ethyl)-2,3-dioxopiperazine-1-carboxamido)-3-hydroxybutanamido)-7-fluoro-2-hydroxy-3,4-dihydro-2H-benzo[e][1,2]oxaborinine-8-carboxylic acid

The title compound was prepared in a similar manner to the synthesis of Example 80, utilizing (tert-butoxycarbonyl)-D-allothreonine in place of (R)-2-((tert-butoxycarbonyl)amino)-3,3-dimethylbutanoic acid. ESI-MS m/z 698 (MH)⁺.

### Example 103: (3R)-3-(2-(2-chloro-5-fluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2-chloro-5-fluoro-4-methoxyphenyl)acetic acid in place of 2-(4-(tert-butoxycarbonyl)-2,6-difluorophenyl)-2-((tert-butoxycarbonyl)amino)acetic acid. ESI-MS m/z 577 (MH)⁺.

### Example 104: (3R)-3-(2-(2,5-difluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2,5-difluoro-4-methoxyphenyl)acetic acid in place of 2-(4-(tert-butoxycarbonyl)-2,6-difluorophenyl)-2-((tert-butoxycarbonyl)amino)acetic acid. ESI-MS m/z 561 (MH)⁺.

### Example 105: (3R)-3-(2-(2,6-difluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2,6-difluoro-4-methoxyphenyl)acetic acid in place of 2-(4-(tert-butoxycarbonyl)-2,6-difluorophenyl)-2-((tert-butoxycarbonyl)amino)acetic acid. ESI-MS m/z 561 (MH)⁺.

### Example 106: (3R)-3-(2-(2,6-difluoro-3,5-dihydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

In a similar manner to the synthesis of Example 17, utilizing 2,6-difluoro-3,5-dimethoxybenzaldehyde in place of 4-methoxy-3-nitrobenzaldehyde in Step 1, the title compound was obtained after reversed phase HPLC purification. ESI-MS m/z 577 (M+1)⁺.

### Example 107: (3R)-3-(2-(4-ethyl-2,3-dioxopiperazine-1-carboxamido)-2-(3-((sulfamoylamino)methyl)phenyl)acetamido)-2-hydroxy-3,4-dihydro-2H-benzo[e][1,2]oxaborinine-8-carboxylic acid

By following the procedures described in Step 8 of Example 15, utilizing 2-amino-2-(3-(((tert-butoxycarbonyl)amino)methyl)phenyl)acetic acid in place of 2-amino-2-(4-(tert-butoxycarbonyl)phenyl)acetic acid, and coupling the resulting acid with the chloride intermediate (prepared as previous reported, WO 2014/089365) by following the General method C, and then removing Boc with HCl, followed by acylation with benzyl (chlorosulfonyl)carbamate and deprotection with BBr₃, the title compound was prepared. ESI-MS m/z 617 (M+H)⁺.

### Example 108: (3R)-3-(2-(3-(aminomethyl)phenyl)-2-(4-ethyl-2,3-dioxopiperazine-1-carboxamido)acetamido)-2-hydroxy-3,4-dihydro-2H-benzo[e][1,2]oxaborinine-8-carboxylic acid

By following the procedures described in Step 8 of Example 15, utilizing 2-amino-2-(3-(((tert-butoxycarbonyl)amino)methyl)phenyl)acetic acid in place of 2-amino-2-(4-(tert-butoxycarbonyl)phenyl)acetic acid, and coupling the resulting acid with the chloride intermediate (prepared as previous reported, WO 2014/089365) by following the General method C, and then deprotection with BBr₃, the title compound was prepared. ESI-MS m/z 538 (M+H)⁺.

### Example 109: (3R)-3-(2-(4-(2-aminoethyl)-2,3-dioxopiperazine-1-carboxamido)-2-(2-chloro-5-fluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2-chloro-5-fluoro-4-methoxyphenyl)acetic acid in place of 2-(4-(tert-butoxycarbonyl)-2,6-difluorophenyl)-2-((tert-butoxycarbonyl)amino)acetic acid. ESI-MS m/z 592 (MH)⁺.

### Example 110: (R)-3-((S)-2-(4-(2-aminoethyl)-2,3-dioxopiperazine-1-carboxamido)-2-(2,6-difluoro-4-hydroxyphenyl)acetamido)-2-hydroxy-3,4-dihydro-2H-benzo[e][1,2]oxaborinine-8-carboxylic acid

The title compound was prepared in a similar manner to the synthesis Example 157, utilizing 2-((tert-butoxycarbonyl)amino)-2-(2,6-difluoro-4-methoxyphenyl)acetic acid in place of 2-(4-(tert-butoxycarbonyl)-2,6-difluorophenyl)-2-((tert-butoxycarbonyl)amino)acetic acid, isolated as the second eluting peak. ESI-MS m/z 576 (MH)⁺.

### Example 111: (R)-3-((R)-2-(4-(2-aminoethyl)-2,3-dioxopiperazine-1-carboxamido)-2-(2,6-difluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 110, the title compound was prepared after reversed phase HPLC purification, isolated as the first eluting peak. ESI-MS m/z 576 (MH)⁺.

### Example 112: (3R)-3-(2-(4-ethyl-2,3-dioxopiperazine-1-carboxamido)-2-(5-hydroxypyridin-3-yl)acetamido)-2-hydroxy-3,4-dihydro-2H-benzo[e][1,2]oxaborinine-8-carboxylic acid

The title compound was prepared in a similar manner to the synthesis of Example 105, utilizing 2-((tert-butoxycarbonyl)amino)-2-(5-methoxypyridin-3-yl)acetic acid in place of 2-((tert-butoxycarbonyl)amino)-2-(2,6-difluoro-4-methoxyphenyl)acetic acid. ESI-MS m/z 526 (MH)⁺.

### Example 113: (3R)-3-(2-(3-carboxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

By following the procedures described in Step 8 of Example 15, utilizing 2-amino-2-(3-(tert-butoxycarbonyl)phenyl)acetic acid in place of 2-amino-2-(4-(tert-butoxycarbonyl)phenyl)acetic acid, and coupling the resulting acid with the chloride intermediate (prepared as previous reported, WO 2014/089365) by following the General method C, and then deprotection with TMSI, the title compound was prepared. ESI-MS m/z 553 (M+H)⁺.

### Example 114: (R)-3-((R)-2-(4-(2-aminoethyl)-2,3-dioxopiperazine-1-carboxamido)-2-(3-(sulfamoylamino)phenyl)acetamido)-2-hydroxy-3,4-dihydro-2H-benzo[e][1,2]oxaborinine-8-carboxylic acid

### Example 115: (R)-3-((S)-2-(4-(2-aminoethyl)-2,3-dioxopiperazine-1-carboxamido)-2-(3-(sulfamoylamino)phenyl)acetamido)-2-hydroxy-3,4-dihydro-2H-benzo[e][1,2]oxaborinine-8-carboxylic acid

By following procedures as described for Example 17, utilizing 2-((tert-butoxycarbonyl)amino)-2-(3-nitrophenyl)acetic acid in place of 2-((tert-butoxycarbonyl)amino)-2-(4-methoxy-3-nitrophenyl)acetic acid in Step 2, and then changing the reaction sequence as follows: i) hydrogenation followed by acylation with benzyl (chlorosulfonyl)carbamate; ii) treated with HCl to remove Boc followed by acylation with tert-butyl (2-(4-(chlorocarbonyl)-2,3-dioxopiperazin-1-yl)ethyl)carbamate; iii) treated with excess BBr₃. The title compound, Example 114 was isolated as the first eluting peak, and Example 115 was isolated as the second eluting peak. ESI-MS m/z 618 (M+1)⁺.

### Example 116: (3R)-3-(2-(4-(2-fluoroethyl)-2,3-dioxopiperazine-1-carboxamido)-2-(3-(sulfamoylamino)phenyl)acetamido)-2-hydroxy-3,4-dihydro-2H-benzo[e][1,2]oxaborinine-8-carboxylic acid

In a similar manner to the synthesis of Example 114, 115, utilizing 4-(2-fluoroethyl)-2,3-dioxopiperazine-1-carbonyl chloride in place of tert-butyl (2-(4-(chlorocarbonyl)-2,3-dioxopiperazin-1-yl)ethyl)carbamate, the title compound was obtained after reversed phase HPLC purification. ESI-MS m/z 621 (M+1)⁺.

### Example 117: (R)-3-((R)-2-(4-(3-aminopropyl)-2,3-dioxopiperazine-1-carboxamido)-2-(3-(sulfamoylamino)phenyl)acetamido)-2-hydroxy-3,4-dihydro-2H-benzo[e][1,2]oxaborinine-8-carboxylic acid

### Example 118: (R)-3-((S)-2-(4-(3-aminopropyl)-2,3-dioxopiperazine-1-carboxamido)-2-(3-(sulfamoylamino)phenyl)acetamido)-2-hydroxy-3,4-dihydro-2H-benzo[e][1,2]oxaborinine-8-carboxylic acid

In a similar manner to the synthesis of Example 114, 115, utilizing tert-butyl (3-(4-(chlorocarbonyl)-2,3-dioxopiperazin-1-yl)propyl)carbamate in place of tert-butyl (2-(4-(chlorocarbonyl)-2,3-dioxopiperazin-1-yl)ethyl)carbamate, Example 117 and Example 118 were prepared after reversed phase HPLC purification. ESI-MS m/z 632 (M+1)⁺.

### Example 119: (3R)-3-(2-(2,6-difluoro-4-hydroxyphenyl)-2-(4-(2-fluoroethyl)-2,3-dioxopiperazine-1-carboxamido)acetamido)-2-hydroxy-3,4-dihydro-2H-benzo[e][1,2]oxaborinine-8-carboxylic acid

In a similar manner to the synthesis of Example 105, utilizing 4-(2-fluoroethyl)-2,3-dioxopiperazine-1-carbonyl chloride in place of 4-ethyl-2,3-dioxopiperazine-1-carbonyl chloride, the title compound was prepared after reversed phase HPLC purification. ESI-MS m/z 579 (M+1)⁺.

### Example 120: (3R)-3-(2-(4-((1-aminocyclopropyl)methyl)-2,3-dioxopiperazine-1-carboxamido)-2-(3-(sulfamoylamino)phenyl)acetamido)-2-hydroxy-3,4-dihydro-2H-benzo[e][1,2]oxaborinine-8-carboxylic acid

In a similar manner to the synthesis of Example 114, 115, utilizing tert-butyl (1-((4-(chlorocarbonyl)-2,3-dioxopiperazin-1-yl)methyl)cyclopropyl)carbamate (which was prepared in a similar manner to tert-butyl (2-(4-(chlorocarbonyl)-2,3-dioxopiperazin-1-yl)ethyl)carbamate) in place of tert-butyl (2-(4-(chlorocarbonyl)-2,3-dioxopiperazin-1-yl)ethyl)carbamate, Example 120 was obtained after reversed phase HPLC purification. ESI-MS m/z 644 (M+1)⁺.

### Example 121: (R)-3-((R)-2-(4-((1-aminocyclopropyl)methyl)-2,3-dioxopiperazine-1-carboxamido)-2-(2,6-difluoro-4-hydroxyphenyl)acetamido)-2-hydroxy-3,4-dihydro-2H-benzo[e][1,2]oxaborinine-8-carboxylic acid

### Example 122: (R)-3-((S)-2-(4-((1-aminocyclopropyl)methyl)-2,3-dioxopiperazine-1-carboxamido)-2-(2,6-difluoro-4-hydroxyphenyl)acetamido)-2-hydroxy-3,4-dihydro-2H-benzo[e][1,2]oxaborinine-8-carboxylic acid

In a similar manner to the synthesis of Example 105, utilizing tert-butyl (1-((4-(chlorocarbonyl)-2,3-dioxopiperazin-1-yl)methyl)cyclopropyl)carbamate in place of 4-ethyl-2,3-dioxopiperazine-1-carbonyl chloride, the title compound, Example 121 was isolated as the first eluting peak, Example 122 was isolated as the second eluting peak after reversed phase HPLC purification. ESI-MS m/z 602 (M+1)⁺.

### Example 123: (R)-3-((S)-2-(4-carboxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

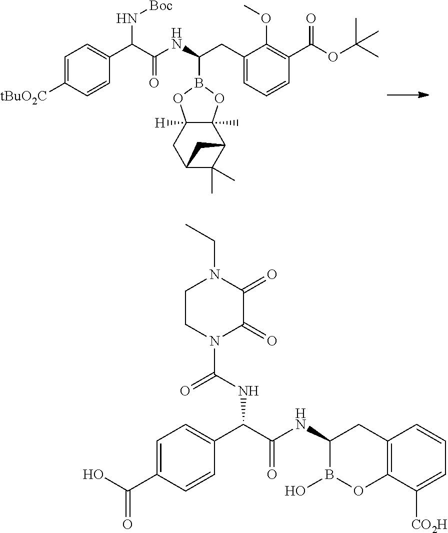 [SMILES: COc1c(C[C@H](NC(=O)C(NC(=O)OC(C)(C)C)c2ccc(C(=O)OC(C)(C)C)cc2)B2O[C@@H]3C[C@@H]4C[C@@H](C4(C)C)[C@]3(C)O2)cccc1C(=O)OC(C)(C)C ; CCN1CCN(C(=O)N[C@H](C(=O)N[C@H]2Cc3cccc(C(=O)O)c3OB2O)c2ccc(C(=O)O)cc2)C(=O)C1=O]

The title compound was prepared in a similar manner to the synthesis of Example 1, utilizing 4-ethyl-2,3-dioxopiperazine-1-carbonyl chloride in place of tert-butyl (3-(4-(chlorocarbonyl)-2,3-dioxopiperazin-1-yl)propyl)carbamate, isolated as the first eluting peak. ESI-MS m/z 553.2 (M+H)⁺.

### Example 124: (R)-3-((R)-2-(4-carboxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

In a same manner to the synthesis of Example 123, the title compound was prepared after reversed phase HPLC purification, isolated as the second eluting peak. ESI-MS m/z 553.2 (M+H)⁺.

### Example 125: (R)-3-((S)-2-(2-chloro-6-fluoro-3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of tert-butyl 3-((2R)-2-(2-((tert-butoxycarbonyl)amino)-2-(2-chloro-6-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

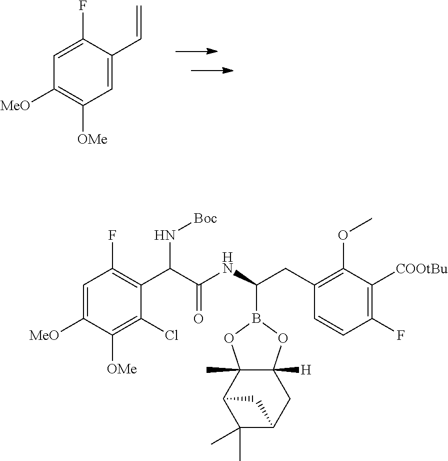

The title compound was prepared in a same manner to the synthesis of Example 25. ESI-MS m/z 793.3 (M+H)⁺.

### Step 2: Synthesis of (R)-3-((S)-2-(2-chloro-6-fluoro-3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

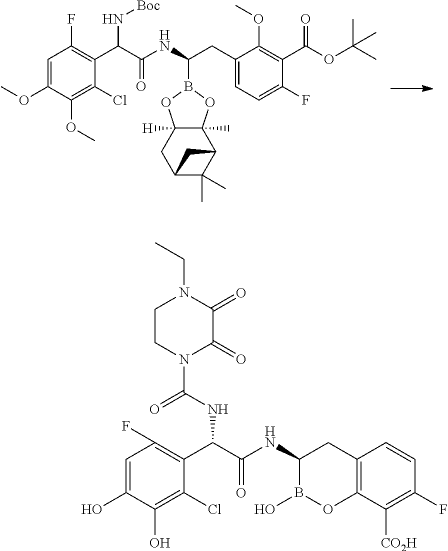 [SMILES: COc1cc(F)c(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; CCN1CCN(C(=O)N[C@H](C(=O)N[C@H]2Cc3ccc(F)c(C(=O)O)c3OB2O)c2c(F)cc(O)c(O)c2Cl)C(=O)C1=O]

The title compound was prepared in a similar manner to the synthesis of Example 1, utilizing 4-ethyl-2,3-dioxopiperazine-1-carbonyl chloride in place of tert-butyl (3-(4-(chlorocarbonyl)-2,3-dioxopiperazin-1-yl)propyl)carbamate, isolated as the first eluting peak. ESI-MS m/z 611.1 (M+H)⁺.

### Example 126: (R)-3-((R)-2-(2,6-difluoro-4-hydroxyphenyl)-2-(4-(2-fluoroethyl)-2,3-dioxopiperazine-1-carboxamido)acetamido)-2-hydroxy-3,4-dihydro-2H-benzo[e][1,2]oxaborinine-8-carboxylic acid

Example 119 was subjected to reversed phase HPLC purification, the title compound was isolated as the second eluting peak. ESI-MS m/z 579 (M+1).

### Example 127: (R)-3-((R)-2-(4-(2-aminoethyl)-2,3-dioxopiperazine-1-carboxamido)-2-(2,6-difluoro-4-hydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 111, utilizing tert-butyl 6-fluoro-3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate and was isolated in the final purification step using a Zorbax column as the first eluting peak. ESI-MS m/z 594 (MH)⁺.

### Example 128: (R)-3-((S)-2-(4-(2-aminoethyl)-2,3-dioxopiperazine-1-carboxamido)-2-(2,6-difluoro-4-hydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 127 and was isolated in the final purification step using a Zorbax column as the second eluting peak. ESI-MS m/z 594 (MH)⁺.

### Example 129: (3R)-3-(2-(2,6-difluoro-4-hydroxyphenyl)-2-(4-(2-fluoroethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 127, utilizing 4-(2-fluoroethyl)-2,3-dioxopiperazine-1-carbonyl chloride in place of tert-butyl (2-(4-(chlorocarbonyl)-2,3-dioxopiperazin-1-yl)ethyl)carbamate. ESI-MS m/z 597 (MH)⁺.

### Example 130: (3R)-3-(2-(2,6-difluoro-4-hydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 105, utilizing tert-butyl 6-fluoro-3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate. ESI-MS m/z 579 (MH)⁺.

### Example 131: (3R)-3-(2-(4-(2-chloro-6-fluoro-3,4-dihydroxybenzyl)-2,3-dioxopiperazine-1-carboxamido)-2-(3-(sulfamoylamino)phenyl)acetamido)-2-hydroxy-3,4-dihydro-2H-benzo[e][1,2]oxaborinine-8-carboxylic acid

### Step 1. Synthesis of 3-chloro-2-(chloromethyl)-1-fluoro-4,5-dimethoxybenzene

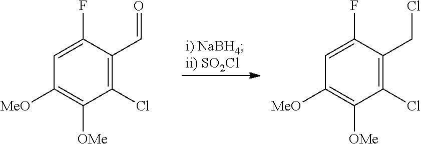 [SMILES: COc1cc(F)c(C=O)c(Cl)c1OC ; COc1cc(F)c(CCl)c(Cl)c1OC]

To 2-chloro-6-fluor-3,4-dimethoxybenzaldehyde (the product of Step 2 of Example 25) (6.98 g, 32 mmol) in MeOH (100 mL) was added NaBH₄ (1.98 g, 52 mmol). The reaction mixture was stirred at RT for 1.5 h, then quenched with water, concentrated in vacuo, and extracted with DCM. The combined organic extracts were dried over Na₂SO₄, concentrated in vacuo. This crude product was dissolved in DCM (100 mL). To this solution was added pyridine (2.6 mL, 32.1 mmol), followed by dropwise addition of thionyl chloride (2.6 mL, 35.6 mmol). The reaction mixture was stirred at RT for 1 h, quenched with water. The organic layer was separated, dried over Na₂SO₄, and concentrated in vacuo to yield the title compound which was used directly for the next step without further purification.

### Step 2. Synthesis of 4-(2-chloro-6-fluoro-3,4-dimethoxybenzyl)-2,3-dioxopiperazine-1-carbonyl chloride

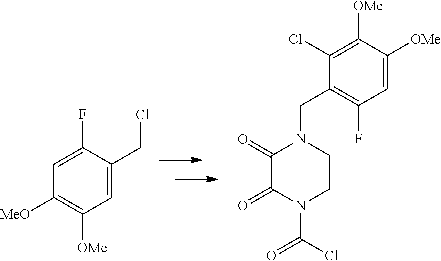 [SMILES: COc1cc(F)c(CCl)cc1OC ; COc1cc(F)c(CN2CCN(C(=O)Cl)C(=O)C2=O)c(Cl)c1OC]

In a similar manner to the synthesis of tert-butyl (2-(4-(chlorocarbonyl)-2,3-dioxopiperazin-1-yl)ethyl)carbamate as described in Step 1 of Example 26, the crude title compound was prepared from the above chloride, which was used directly for the next step without further purification.

### Step 3. Synthesis of (3R)-3-(2-(4-(2-chloro-6-fluoro-3,4-dihydroxybenzyl)-2,3-dioxpiperazine-1-carboxamido)-2-(3-(sulfamoylamino)phenyl)acetamido)-2-hydroxy-3,4-dihydro-2H-benzo[e][1,2]oxaborinine-8-carboxylic acid

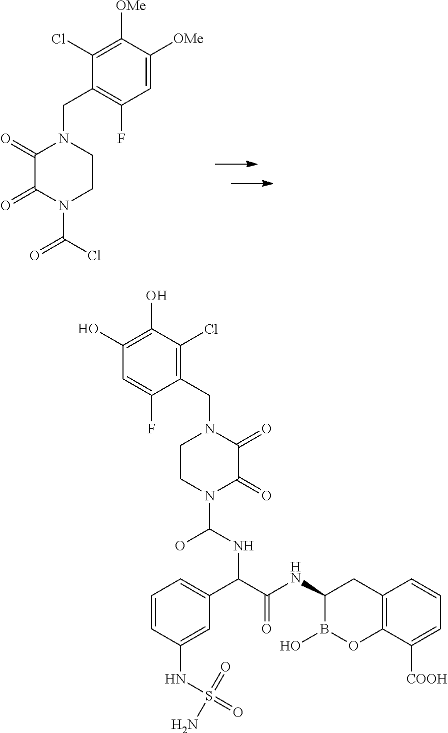 [SMILES: COc1cc(F)c(CN2CCN(C(=O)Cl)C(=O)C2=O)c(Cl)c1OC ; NS(=O)(=O)Nc1cccc(C(NC(O)N2CCN(Cc3c(F)cc(O)c(O)c3Cl)C(=O)C2=O)C(=O)N[C@H]2Cc3cccc(C(=O)O)c3OB2O)c1]

In a similar manner to the synthesis of Example 114, 115, utilizing the above 4-(2-chloro-6-fluoro-3,4-dimethoxybenzyl)-2,3-dioxopiperazine-1-carbonyl chloride in place of tert-butyl (2-(4-(chlorocarbonyl)-2,3-dioxopiperazin-1-yl)ethyl)carbamate, Example 131 was prepared. ESI-MS m/z 749/751 (MH/MH+2)⁺.

### Example 132: (3R)-3-(2-(4-(2-chloro-6-fluoro-3,4-dihydroxybenzyl)-2,3-dioxopiperazine-1-carboxamido)-2-(3-hydroxy-5-sulfamoylphenyl)acetamido)-2-hydroxy-3,4-dihydro-2H-benzo[e][1,2]oxaborinine-8-carboxylic acid

In a similar manner to the synthesis of Example 17, utilizing 3-formyl-5-methoxybenzenesulfonamide in place of 4-methoxy-3-nitrobenzaldehyde in Step 1, and utilizing 4-(2-chloro-6-fluoro-3,4-dimethoxybenzyl)-2,3-dioxopiperazine-1-carbonyl chloride in place of 4-ethyl-2,3-dioxopiperazine-1-carbonyl chloride in Step 4, the title compound was obtained after reversed phase HPLC purification. ESI-MS m/z 750/752 (MH/MH+2)⁺.

### Example 133: (R)-3-((R)-2-(4-((1-aminocyclopropyl)methyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 42, utilizing tert-butyl (1-((4-(chlorocarbonyl)-2,3-dioxopiperazin-1-yl)methyl)cyclopropyl)carbamate in place of 4-ethyl-2,3-dioxopiperazine-1-carbonyl chloride, the title compound was isolated as the first eluting peak after reversed phase HPLC purification. ESI-MS m/z 652/654 (MH/MH+2)⁺.

### Example 134: (R)-3-((R)-2-(4-((1-aminocyclopropyl)methyl)-2,3-dioxopiperazine-1-carboxamido)-2-(2-chloro-6-fluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 26, utilizing tert-butyl (1-((4-(chlorocarbonyl)-2,3-dioxopiperazin-1-yl)methyl)cyclopropyl)carbamate in place of tert-butyl (2-(4-(chlorocarbonyl)-2,3-dioxopiperazin-1-yl)ethyl)carbamate, the title compound was isolated as the first eluting peak after reversed phase HPLC purification. ESI-MS m/z 652/654 (MH/MH+2)⁺.

### Example 135: (R)-3-((R)-2-(2,6-difluoro-4-hydroxyphenyl)-2-(3-(methylsulfonyl)-2-oxoimidazolidine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2,6-difluoro-4-methoxyphenyl)acetic acid in place of 2-(4-(tert-butoxycarbonyl)-2,6-difluorophenyl)-2-((tert-butoxycarbonyl)amino)acetic acid, isolated as the second eluting peak. ESI-MS m/z 583 (MH).

### Example 136: (R)-3-((S)-2-(2,6-difluoro-4-hydroxyphenyl)-2-(3-(methylsulfonyl)-2-oxoimidazolidine-1-carboxamido)acetamido)-2-hydroxy-3,4-dihydro-2H-benzo[e][1,2]oxaborinine-8-carboxylic acid

The title compound was prepared in a similar manner to the synthesis of Example 135, the title compound was prepared after reversed phase HPLC purification, isolated as the first eluting peak. ESI-MS m/z 583 (MH)⁺.

### Example 137: (3R)-3-(2-(4-carboxy-2,5-difluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-(4-(tert-butoxycarbonyl)-2,5-difluorophenyl)-2-((tert-butoxycarbonyl)amino)acetic acid in place of 2-(4-(tert-butoxycarbonyl)-2,6-difluorophenyl)-2-((tert-butoxycarbonyl)amino)acetic acid. ESI-MS m/z 589 (MH)⁺.

### Example 138: (3R)-3-(2-(4-carboxy-5-chloro-2-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-(4-(tert-butoxycarbonyl)-5-chloro-2-fluorophenyl)-2-((tert-butoxycarbonyl)amino)acetic acid in place of 2-(4-(tert-butoxycarbonyl)-2,6-difluorophenyl)-2-((tert-butoxycarbonyl)amino)acetic acid. ESI-MS m/z 605 (MH)⁺.

### Example 139: (R)-3-((S)-2-(4-carboxy-2,3-difluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-(4-(tert-butoxycarbonyl)-2,3-difluorophenyl)-2-((tert-butoxycarbonyl)amino)acetic acid in place of 2-(4-(tert-butoxycarbonyl)-2,6-difluorophenyl)-2-((tert-butoxycarbonyl)amino)acetic acid, isolated as the first eluting peak. ESI-MS m/z 589 (MH)⁺.

### Example 140: (R)-3-((R)-2-(4-carboxy-2,3-difluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 139, the title compound was prepared after reversed phase HPLC purification, isolated as the second eluting peak. ESI-MS m/z 589 (MH)⁺.

### Example 141: (3R)-3-(2-(4-carboxy-3,5-difluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-(4-(tert-butoxycarbonyl)-3,5-difluorophenyl)-2-((tert-butoxycarbonyl)amino)acetic acid in place of 2-(4-(tert-butoxycarbonyl)-2,6-difluorophenyl)-2-((tert-butoxycarbonyl)amino)acetic acid. ESI-MS m/z 589 (MH)⁺.

### Example 142:(3R)-3-(2-(4-(2-aminoethyl)-2,3-dioxopiperazine-1-carboxamido)-2-(4-carboxy-2-hydroxyphenyl)acetamido)-2-hydroxy-3,4-dihydro-2H-benzo[e][1,2]oxaborinine-8-carboxylic acid

The title compound was prepared in a similar manner to the synthesis of Example 157, utilizing 2-(4-(tert-butoxycarbonyl)-2-methoxyphenyl)-2-((tert-butoxycarbonyl)amino)acetic acid in place of 2-(4-(tert-butoxycarbonyl)-2,6-difluorophenyl)-2-((tert-butoxycarbonyl)amino)acetic acid. ESI-MS m/z 584 (MH)⁺.

### Example 143: (R)-3-((S)-2-(4-carboxy-2-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-(4-(tert-butoxycarbonyl)-2-methoxyphenyl)-2-((tert-butoxycarbonyl)amino)acetic acid in place of 2-(4-(tert-butoxycarbonyl)-2,6-difluorophenyl)-2-((tert-butoxycarbonyl)amino)acetic acid, isolated as the first eluting peak. ESI-MS m/z 569 (NM)⁺.

### Example 144: (R)-3-((R)-2-(4-carboxy-2-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 143, the title compound was prepared after reversed phase HPLC purification, isolated as the second eluting peak. ESI-MS m/z 569 (MH)⁺.

### Example 145: (3R)-3-(2-(4-carboxy-2-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-(4-(tert-butoxycarbonyl)-2-fluorophenyl)-2-((tert-butoxycarbonyl)amino)acetic acid in place of 2-(4-(tert-butoxycarbonyl)-2,6-difluorophenyl)-2-((tert-butoxycarbonyl)amino)acetic acid. ESI-MS m/z 571 (MH)⁺.

### Example 146: (R)-3-((R)-2-(4-carboxy-3-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-(4-(tert-butoxycarbonyl)-3-fluorophenyl)-2-((tert-butoxycarbonyl)amino)acetic acid in place of 2-(4-(tert-butoxycarbonyl)-2,6-difluorophenyl)-2-((tert-butoxycarbonyl)amino)acetic acid, isolated as the second eluting peak ESI-MS m/z 571 (MH)⁺.

### Example 147: (R)-3-((S)-2-(4-carboxy-3-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 146, the title compound was prepared after reversed phase HPLC purification, isolated as the first eluting peak. ESI-MS m/z 571 (MH)⁺.

### Example 148: (R)-3-((R)-2-(4-carboxy-2-chloro-5-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of 2-(4-(tert-butoxycarbonyl)-2-chloro-5-fluorophenyl)-2-((tert-butoxycarbonyl)amino)acetic acid

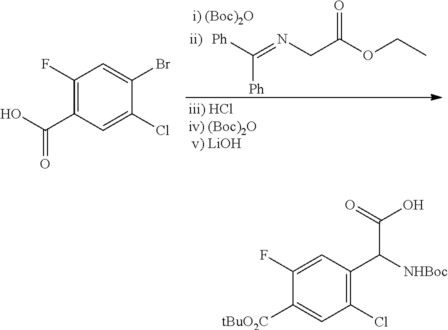

In a same manner to the synthesis of Step 1, 2, 3, 4, 5 of Example 157, the title compound was prepared from 4-bromo-5-chloro-2-fluorobenzoic acid. ESI-MS m/z 427 (M+Na)⁺.

### Step 2. Synthesis of tert-butyl 4-(2-(((R)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-((tert-butoxycarbonyl)amino)-2-oxoethyl)-5-chloro-2-fluorobenzoate

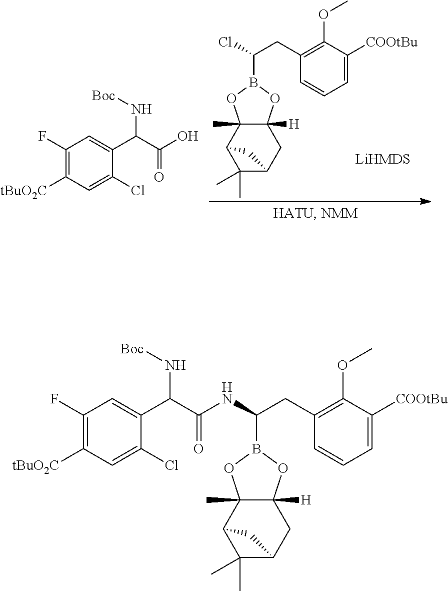

By following the General coupling method C, the title compound was prepared from the above acid and the chloride intermediate (prepared as previous reported, WO 2014/089365). ESI-MS m/z 759 (M+H-56)⁺.

### Step 3. Synthesis of (R)-3-((R)-2-(4-carboxy-2-chloro-5-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

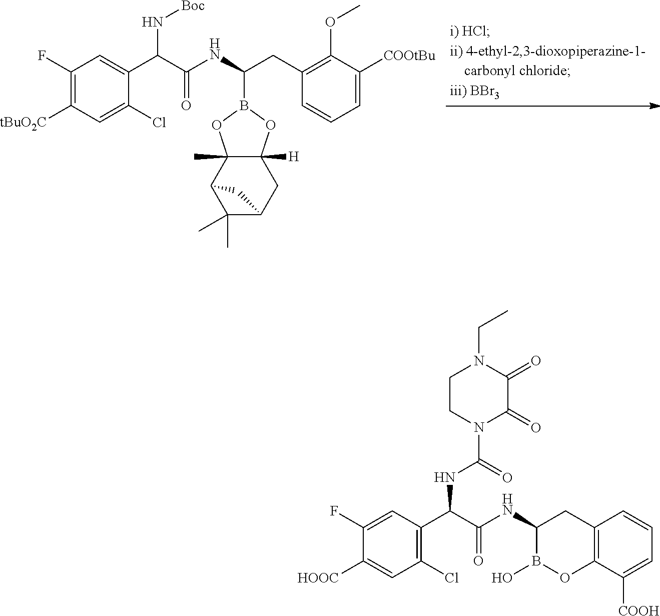

In a similar manner to the synthesis of Example 26, the title compound was prepared after reversed phase HPLC purification, isolated as the second eluting peak. ESI-MS m/z 605 (M+H)⁺.

### Example 149: (R)-3-((S)-2-(4-carboxy-2-chloro-5-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

In a same manner to the synthesis of Example 86, the title compound was prepared after reversed phase HPLC purification, isolated as the first eluting peak. ESI-MS m/z 605 (M+H)⁺.

### Example 150: (3R)-3-(2-(4-carboxy-2,6-dichlorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of 2-(4-(tert-butoxycarbonyl)-2,6-dichlorophenyl)-2-((tert-butoxycarbonyl)amino)acetic acid

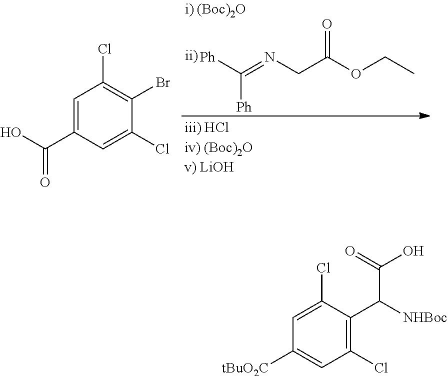 [SMILES: O=C(O)c1cc(Cl)c(Br)c(Cl)c1 ; CCOC(=O)CN=C(c1ccccc1)c1ccccc1 ; CC(C)(C)OC(=O)NC(C(=O)O)c1c(Cl)cc(C(=O)OC(C)(C)C)cc1Cl]

In a same manner to the synthesis of Step 1, 2, 3, 4, 5 of Example 157, the title compound was prepared from 4-bromo-3,5-dichlorobenzoic acid. ESI-MS m/z 443 (M+Na)⁺.

### Step 2. Synthesis of tert-butyl 4-(2-(((R)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-((tert-butoxycarbonyl)amino)-2-oxoethyl)-3,5-dichlorobenzoate

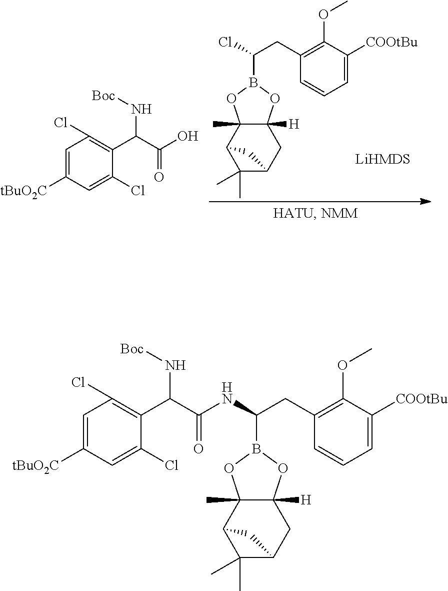

By following the General coupling method C, the title compound was prepared from the above acid and the chloride intermediate (prepared as previous reported, WO 2014/089365). ESI-MS m/z (M+H-56)⁺.

### Step 3. (3R)-3-(2-(4-carboxy-2,6-dichlorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

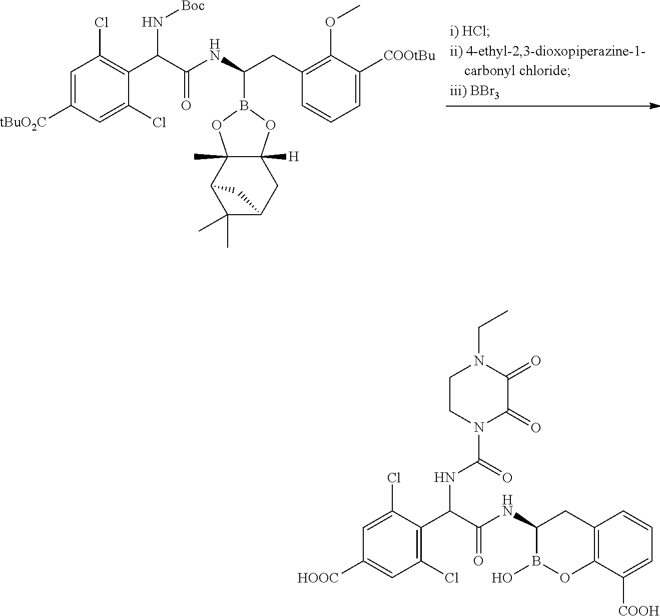

In a similar manner to the synthesis of Example 26, the title compound was prepared after reversed phase HPLC purification. ESI-MS m/z 621. (M+H)⁺.

### Example 151: (R)-3-((R)-2-(4-carboxy-2-chlorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of 2-(4-(tert-butoxycarbonyl)-2-chlorophenyl)-2-((tert-butoxycarbonyl)amino)acetic acid

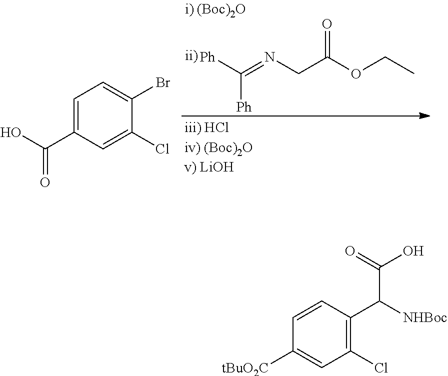 [SMILES: O=C(O)c1ccc(Br)c(Cl)c1 ; CCOC(=O)CN=C(c1ccccc1)c1ccccc1 ; CC(C)(C)OC(=O)NC(C(=O)O)c1ccc(C(=O)OC(C)(C)C)cc1Cl]

In a same manner to the synthesis of Steps 1, 2, 3, 4, and 5 of Example 157, the title compound was prepared from 4-bromo-3-chlorobenzoic acid. ESI-MS m/z 408 (M+Na)⁺.

### Step 2. Synthesis of tert-butyl 3-((2R)-2-(2-(4-(tert-butoxycarbonyl)-2-chlorophenyl)-2-((tert-butoxycarbonyl)amino)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

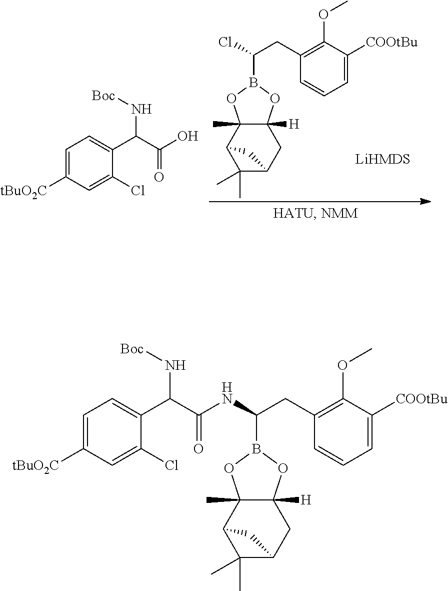

By following the General coupling method C, the title compound was prepared from the above acid and the chloride intermediate (prepared as previous reported, WO 2014/089365). ESI-MS m/z 743 (M+H-56)⁺.

### Step 3. Synthesis of (R)-3-((R)-2-(4-carboxy-2-chlorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

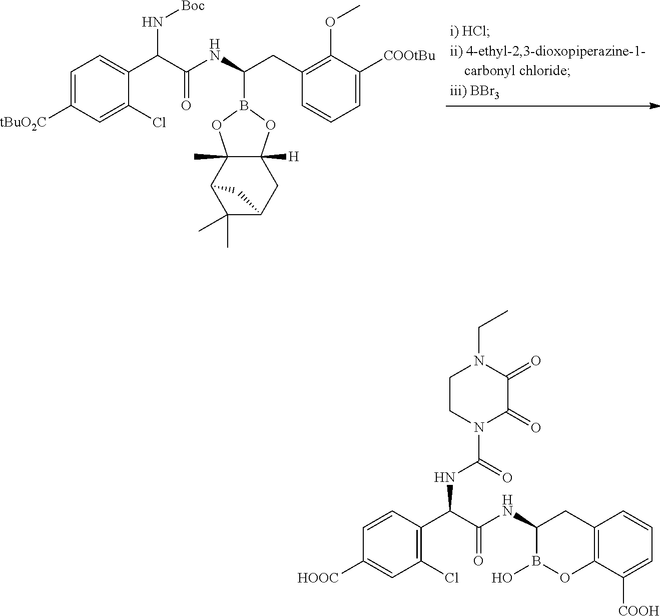

In a similar manner to the synthesis of Example 26, the title compound was prepared after reversed phase HPLC purification, isolated as the second eluting peak. ESI-MS m/z 587 (M+H)⁺.

### Example 152: (R)-3-((S)-2-(4-carboxy-2-chlorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

In a same manner to the synthesis of Example 26, the title compound was prepared after reversed phase HPLC purification, isolated as the first eluting peak. ESI-MS m/z 587 (M+H)⁺.

### Example 153: (3R)-3-(2-(4-(2-aminoethyl)-2,3-dioxopiperazine-1-carboxamido)-2-(4-carboxy-3-chlorophenyl)acetamido)-2-hydroxy-3,4-dihydro-2H-benzo[e][1,2]oxaborinine-8-carboxylic acid

### Step 1. Synthesis of 2-(4-(tert-butoxycarbonyl)-3-chlorophenyl)-2-((tert-butoxycarbonyl)amino)acetic acid

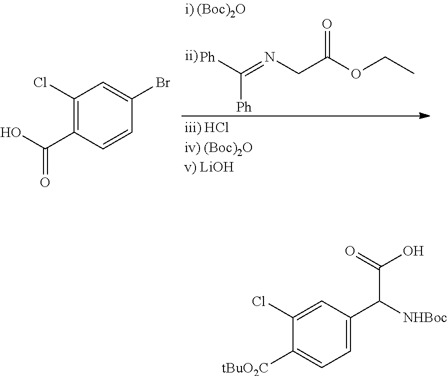 [SMILES: O=C(O)c1ccc(Br)cc1Cl ; CCOC(=O)CN=C(c1ccccc1)c1ccccc1 ; CC(C)(C)OC(=O)NC(C(=O)O)c1ccc(C(=O)OC(C)(C)C)c(Cl)c1]

In a same manner to the synthesis of Step 1, 2, 3, 4, 5 of Example 157, the title compound was prepared from 4-bromo-2-chlorobenzoic acid. ESI-MS m/z 408 (M+Na)⁺.

### Step 2. Synthesis of tert-butyl 3-((2R)-2-(2-(4-(tert-butoxycarbonyl)-3-chlorophenyl)-2-((tert-butoxycarbonyl)amino)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

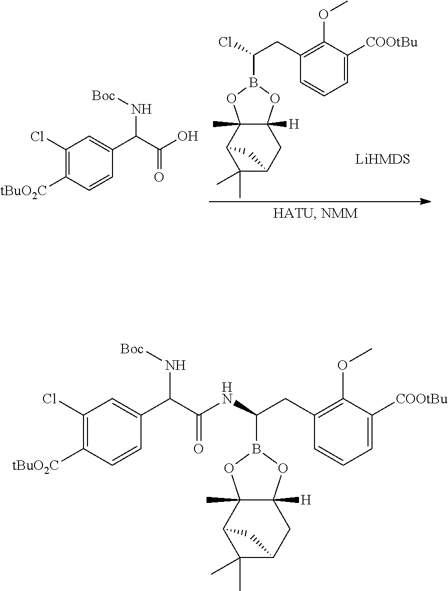

By following the General coupling method C, the title compound was prepared from the above acid and the chloride intermediate (prepared as previous reported, WO 2014/089365). ESI-MS m/z 743 (M+H-56)⁺.

### Step 3. Synthesis of tert-butyl (2-(4-(chlorocarbonyl)-2,3-dioxopiperazin-1-yl)ethyl)carbamate

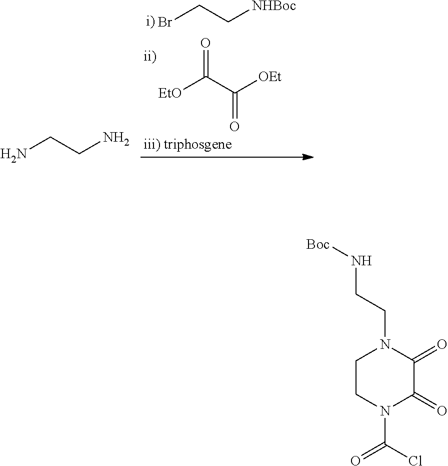

By following the same procedures as described for the synthesis of 4-ethyl-2,3-dioxopiperazine-1-carbonyl chloride in Step 1 of Example 26, the title compound was obtained.

### Step 4. Synthesis of (3R)-3-(2-(4-(2-aminoethyl)-2,3-dioxopiperazine-1-carboxamido)-2-(4-carboxy-3-chlorophenyl)acetamido)-2-hydroxy-3,4-dihydro-2H-benzo[e][1,2]oxaborinine-8-carboxylic acid

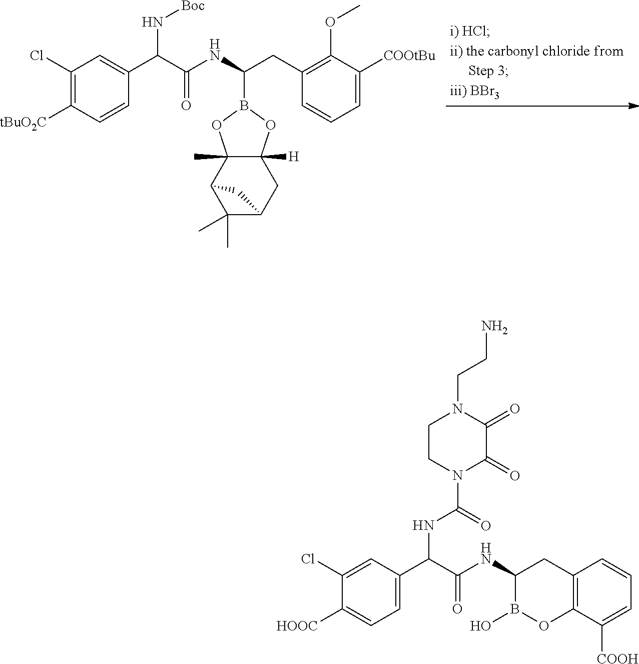

In a similar manner to the synthesis of Example 26, the title compound was prepared after reversed phase HPLC purification. ESI-MS m/z 602 (M+H)⁺.

### Example 154: (R)-3-((R)-2-(4-carboxy-3-chlorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of 2-(4-(tert-butoxycarbonyl)-3-chlorophenyl)-2-((tert-butoxycarbonyl)amino)acetic acid

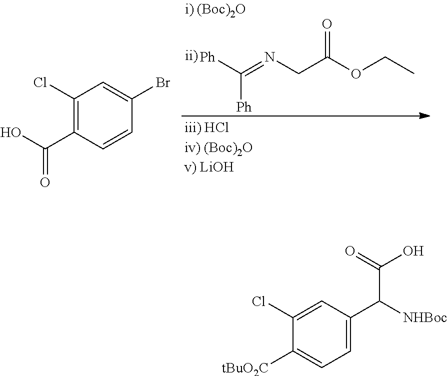 [SMILES: O=C(O)c1ccc(Br)cc1Cl ; CCOC(=O)CN=C(c1ccccc1)c1ccccc1 ; CC(C)(C)OC(=O)NC(C(=O)O)c1ccc(C(=O)OC(C)(C)C)c(Cl)c1]

In a same manner to the synthesis of Step 1, 2, 3, 4, 5 of Example 157, the title compound was prepared from 4-bromo-2-chlorobenzoic acid. ESI-MS m/z 408 (M+Na)⁺.

### Step 2. Synthesis of tert-butyl 3-((2R)-2-(2-(4-(tert-butoxycarbonyl)-3-chlorophenyl)-2-((tert-butoxycarbonyl)amino)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

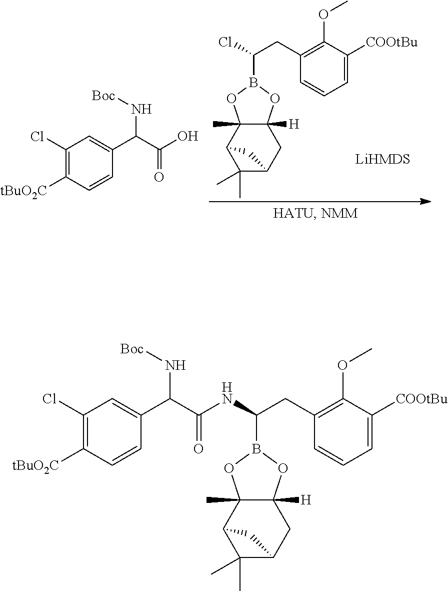

By following the General coupling method C, the title compound was prepared from the above acid and the chloride intermediate (prepared as previous reported, WO 2014/089365). ESI-MS m/z 743 (M-56)⁺.

### Step 3. Synthesis of (R)-3-((R)-2-(4-carboxy-3-chlorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

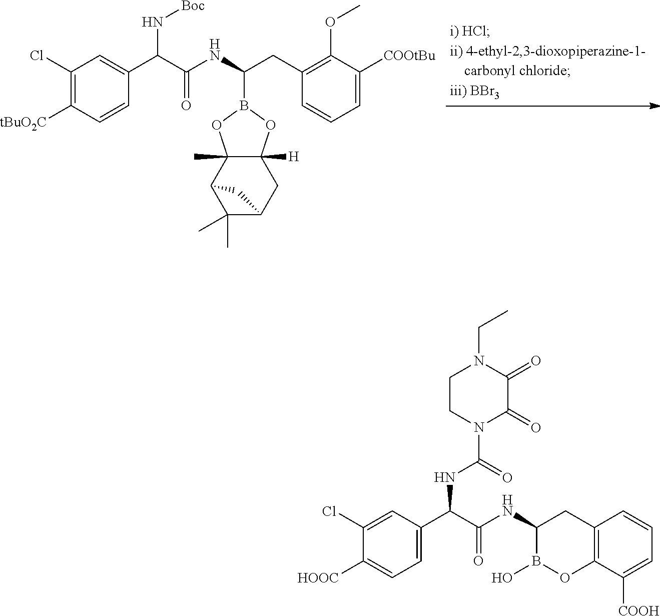

In a same manner to the synthesis of Example 148, the title compound was prepared after reversed phase HPLC purification, isolated as the second eluting peak. ESI-MS m/z 587 (M+H)⁺.

### Example 155: (R)-3-((S)-2-(4-carboxy-3-chlorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

In a same manner to the synthesis of Example 86, the title compound was prepared after reversed phase HPLC purification, isolated as the first eluting peak. ESI-MS m/z 587 (M+H)⁺.

### Example 156: (3R)-3-(2-(2,6-difluoro-4-hydroxyphenyl)-2-(2-oxoimidazolidine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2,6-difluoro-4-methoxyphenyl)acetic acid in place of 2-(4-(tert-butoxycarbonyl)-2,6-difluorophenyl)-2-((tert-butoxycarbonyl)amino)acetic acid. ESI-MS m/z 505 (MH)⁺.

### Example 157: (3R)-3-(2-(4-carboxy-2,6-difluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

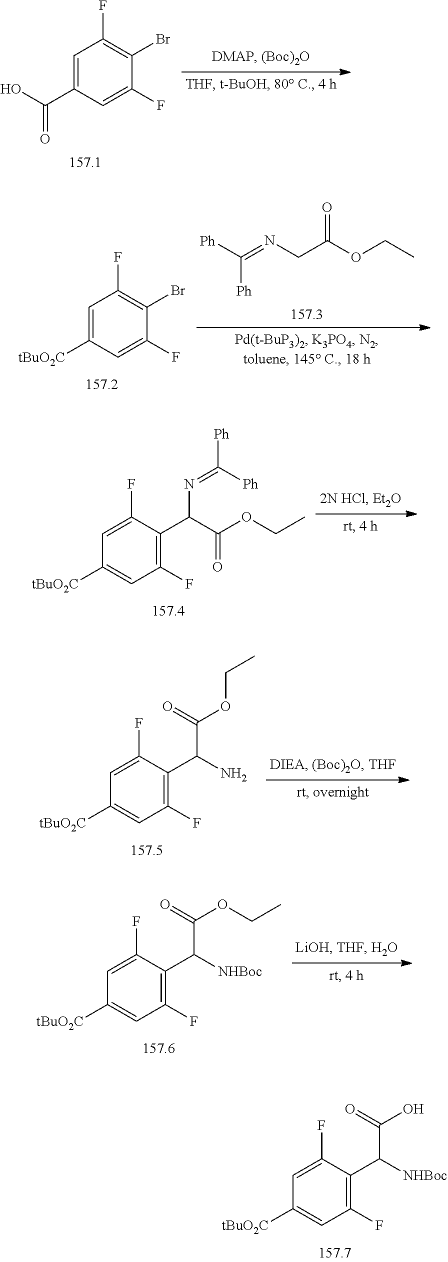

### Step 1: Synthesis of tert-butyl 4-bromo-3,5-difluorobenzoate

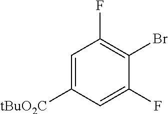 [SMILES: CC(C)(C)OC(=O)c1cc(F)c(Br)c(F)c1]

To a solution of 4-bromo-3,5-difluorobenzoic acid (17.8 g, 75 mmol), t-BuOH (15 mL), (Boc)₂O (33 g, 150 mmol) in THF, DMAP (4.5 g, 37.5 mmol) was slowly added in portions over 20 min (NOTE: the reaction was violent, be careful.) And then the reaction was stirred at 70° C. for 18 h. The reaction was concentrated and purified by FCC (PE:EA=30:1) to give the desired product as an oil, 20 g, 90.4% yield.

### Step 2: Synthesis of tert-butyl 4-(1-((diphenylmethylene)amino)-2-ethoxy-2-oxoethyl)-3,5-difluorobenzoate

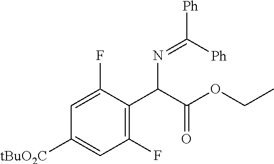 [SMILES: CCOC(=O)C(N=C(c1ccccc1)c1ccccc1)c1c(F)cc(C(=O)OC(C)(C)C)cc1F]

To a solution of tert-butyl 4-bromo-3,5-difluorobenzoate (7.3 g, 25 mmol), Pd(t-BuP₃)₂ (2 g), K₃PO₄ (15.9 g, 75 mmol) in toluene, and then the reaction was stirred at 145° C. for 18 h under N₂. The reaction mixture was concentrated and purified by FCC (PE:EA=12:1) to give the desired product as brown oil, 1.8 g, 15% yield. ESI-MS m/z 479.9 (M+H)⁺.

### Step 3: Synthesis of tert-butyl 4-(1-amino-2-ethoxy-2-oxoethyl)-3,5-difluorobenzoate

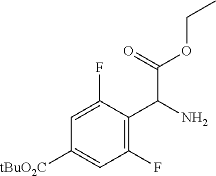 [SMILES: CCOC(=O)C(N)c1c(F)cc(C(=O)OC(C)(C)C)cc1F]

To a solution of tert-butyl 4-(1-((diphenylmethylene)amino)-2-ethoxy-2-oxoethyl)-3,5-difluorobenzoate (2.3 g, 4.8 mmol) in 2N HCl-Et₂O solution (20 mL), and the reaction was stirred at RT for 4 h. 1 mL of H₂O was added. And the reaction mixture was stirred for 2 min, dried over Na₂SO₄, 60 mL EA was added. After filtration, the filtrate was concentrated to give the crude product as brown oil, 2.4 g. ESI-MS m/z 338.1 (M+Na)⁺.

### Step 4: Synthesis of tert-butyl 4-(1-((tert-butoxycarbonyl)amino)-2-ethoxy-2-oxoethyl)-3,5-difluorobenzoate

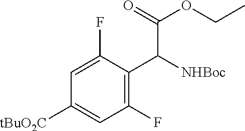 [SMILES: CCOC(=O)C(NC(=O)OC(C)(C)C)c1c(F)cc(C(=O)OC(C)(C)C)cc1F]

To a solution of tert-butyl 4-(1-amino-2-ethoxy-2-oxoethyl)-3,5-difluorobenzoate (2.4 g crude), DIEA (3 mL), (Boc)₂O (3.5 g, 16 mmol) in THF (20 mL) (PS: if the mixture was not dissolved, Some DCM added), and the reaction mixture was stirred at RT for 18 h. The reaction mixture was then concentrated and purified by FCC (PE:EA=20:1%) to give the desired product as yellow oil, 1.6 g, 80% yield. ESI-MS m/z 438.1 (M+Na)⁺.

### Step 5: 2-(4-(tert-butoxycarbonyl)-2,6-difluorophenyl)-2-((tert-butoxycarbonyl)amino)acetic acid

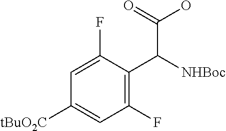 [SMILES: CC(C)(C)OC(=O)NC(C(=O)O)c1c(F)cc(C(=O)OC(C)(C)C)cc1F]

To a solution of tert-butyl 4-(1-((tert-butoxycarbonyl)amino)-2-ethoxy-2-oxoethyl)-3,5-difluorobenzoate (1.6 g, 3.8 mmol) in THF (15 mL) was added LiOH solution (15 mL, 2 N). The reaction mixture was stirred at RT for 4 h. The reaction was acidified by adding HCl aq.to pH=3 and extracted with EA (3*50 mL), dried over Na₂SO₄ and filtered. The filtrate was concentrated and purified by FCC (PE:EA=1:1) to give the desired product as white solid, 1 g, 67% yield. ESI-MS m/z 410.1 (M+Na)⁺.

### Step 6: (3R)-3-(2-(4-carboxy-2,6-difluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

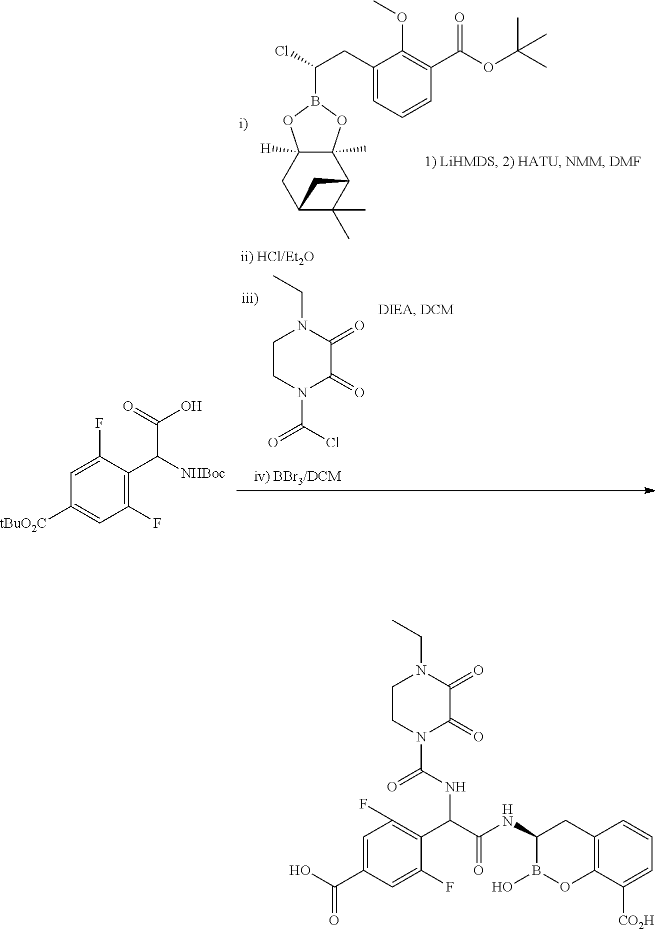 [SMILES: COc1c(C[C@@H](Cl)B2O[C@@H]3C[C@@H]4C[C@@H](C4(C)C)[C@]3(C)O2)cccc1C(=O)OC(C)(C)C ; CC(C)(C)OC(=O)NC(C(=O)O)c1c(F)cc(C(=O)OC(C)(C)C)cc1F ; CCN1CCN(C(=O)Cl)C(=O)C1=O ; CCN1CCN(C(=O)NC(C(=O)N[C@H]2Cc3cccc(C(=O)O)c3OB2O)c2c(F)cc(C(=O)O)cc2F)C(=O)C1=O]

The title compound was prepared in a similar manner to the synthesis of Example 17, utilizing 2-(4-(tert-butoxycarbonyl)-2,6-difluorophenyl)-2-((tert-butoxycarbonyl)amino)acetic acid in place of 2-((tert-butoxycarbonyl)amino)-2-(4-methoxy-3-nitrophenyl)acetic acid in step 2, utilizing tert-butyl 4-(2-(((S)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-((tert-butoxycarbonyl)amino)-2-oxoethyl)-3,5-difluorobenzoate in place of tert-butyl 3-((2R)-2-(2-((tert-butoxycarbonyl)amino)-2-(4-methoxy-3-nitrophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate.in step 3, utilizing tert-butyl 4-(1-amino-2-(((S)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-2-oxoethyl)-3,5-difluorobenzoate in place of tert-butyl 3-((2R)-2-(2-amino-2-(4-methoxy-3-nitrophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate in step 4, utilizing tert-butyl 4-(2-(((S)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-(4-ethyl-2,3-dioxopiperazine-1-carboxamido)-2-oxoethyl)-3,5-difluorobenzoate in place of tert-butyl 3-((2R)-2-(2-(4-ethyl-2,3-dioxopiperazine-1-carboxamido)-2-(4-methoxy-3-nitrophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate in step 6. ESI-MS m/z 589 (MH)⁺.

### Example 158: (R)-3-((S)-2-(4-(2-aminoethyl)-2,3-dioxopiperazine-1-carboxamido)-2-(3,5-difluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(3,5-difluoro-4-methoxyphenyl)acetic acid in place of 2-(4-(tert-butoxycarbonyl)-2,6-difluorophenyl)-2-((tert-butoxycarbonyl)amino)acetic acid, isolated as the second eluting peak ESI-MS m/z 576 (MH)⁺.

### Example 159: (R)-3-((R)-2-(4-(2-aminoethyl)-2,3-dioxopiperazine-1-carboxamido)-2-(3,5-difluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 158, the title compound was prepared after reversed phase HPLC purification, isolated as the first eluting peak. ESI-MS m/z 576 (MH)⁺.

### Example 160: (3R)-3-(2-(3,5-difluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(3,5-difluoro-4-methoxyphenyl)acetic acid in place of 2-(4-(tert-butoxycarbonyl)-2,6-difluorophenyl)-2-((tert-butoxycarbonyl)amino)acetic acid. ESI-MS m/z 561 (MH)⁺.

### Example 161: (R)-3-((R)-2-(3-acetyl-2-oxoimidazolidin-1-yl)-2-(2,6-difluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2,6-difluoro-4-methoxyphenyl)acetic acid in place of 2-(4-(tert-butoxycarbonyl)-2,6-difluorophenyl)-2-((tert-butoxycarbonyl)amino)acetic acid, isolated as the second eluting peak. ESI-MS m/z 504 (MH)⁺.

### Example 162: (R)-3-((S)-2-(3-acetyl-2-oxoimidazolidin-1-yl)-2-(2,6-difluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 161, the title compound was prepared after reversed phase HPLC purification, isolated as the first eluting peak. ESI-MS m/z 504 (MH)⁺.

### Example 163: (3R)-3-(2-(4-(2-aminoethyl)-2,3-dioxopiperazine-1-carboxamido)-2-(3-chloro-5-fluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(3-chloro-5-fluoro-4-methoxyphenyl)acetic acid

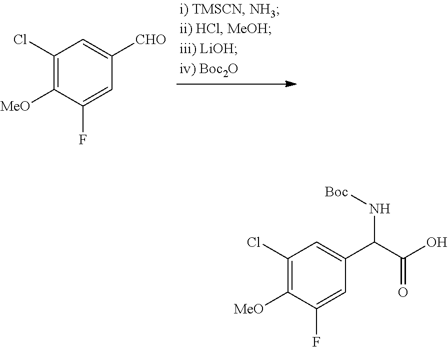 [SMILES: COc1c(F)cc(C=O)cc1Cl ; COc1c(F)cc(C(NC(=O)OC(C)(C)C)C(=O)O)cc1Cl]

In a same manner to the synthesis of Step 1 of Example 17, the title compound was prepared from 3-chloro-5-fluoro-4-methoxybenzaldehyde. ESI-MS m/z 346 (M+Na)⁺.

### Step 2. Synthesis of tert-butyl 3-((2R)-2-(2-((tert-butoxycarbonyl)amino)-2-(3-chloro-5-fluoro-4-hydroxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

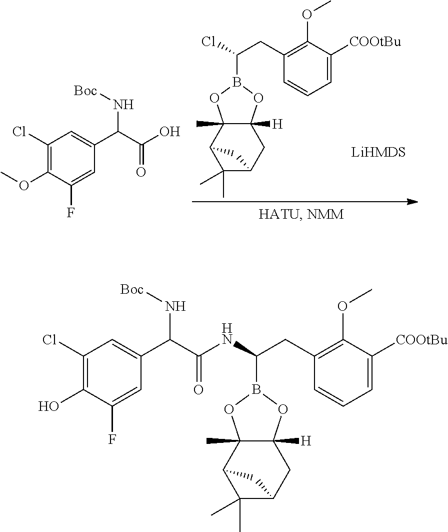

By following the General coupling method C, the title compound was prepared from the above acid and the chloride intermediate (prepared as previous reported, WO 2014/089365). ESI-MS m/z 675 (M+H-56)⁺.

### Step 3. Synthesis of tert-butyl (2-(4-(chlorocarbonyl)-2,3-dioxopiperazin-1-yl)ethyl)carbamate

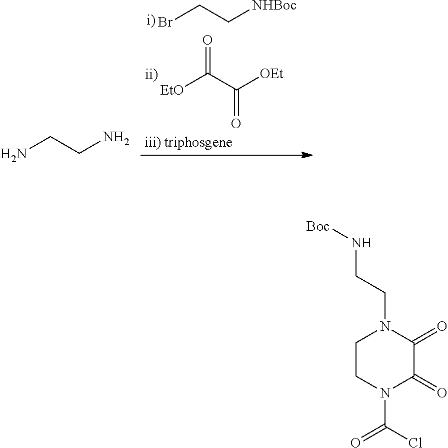

By following the same procedures as described for the synthesis of tert-butyl (2-(4-(chlorocarbonyl)-2,3-dioxopiperazin-1-yl)ethyl)carbamate in Step 1 of Example 26.

### Step 4. Synthesis of (3R)-3-(2-(4-(2-aminoethyl)-2,3-dioxopiperazine-1-carboxamido)-2-(3-chloro-5-fluoro-4-hydroxyphenyl)acetamido)-2-hydroxy-3,4-dihydro-2H-benzo[e][1,2]oxaborinine-8-carboxylic acid

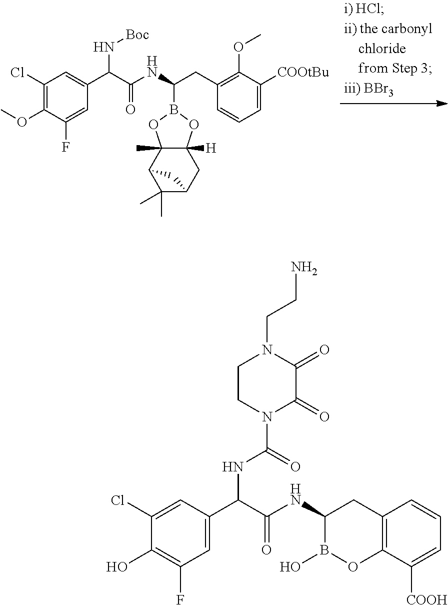

In a similar manner to the synthesis of Example 26, the title compound was prepared after reversed phase HPLC purification. ESI-MS m/z 592 (M+H)⁺.

### Example 164: (3R)-3-(2-(3-chloro-5-fluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(3-chloro-5-fluoro-4-methoxyphenyl)acetic acid

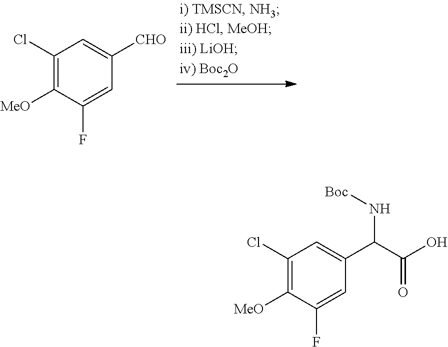 [SMILES: COc1c(F)cc(C=O)cc1Cl ; COc1c(F)cc(C(NC(=O)OC(C)(C)C)C(=O)O)cc1Cl]

In a same manner to the synthesis of Step 1 of Example 17, the title compound was prepared from 3-chloro-5-fluoro-4-methoxybenzaldehyde. ESI-MS m/z 346 (M+Na)⁺.

### Step 2. Synthesis of tert-butyl 3-((2R)-2-(2-((tert-butoxycarbonyl)amino)-2-(3-chloro-5-fluoro-4-hydroxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

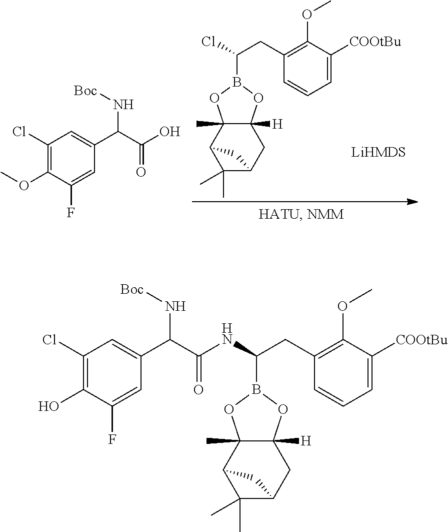

By following the General coupling method C, the title compound was prepared from the above acid and the chloride intermediate (prepared as previous reported, WO 2014/089365). ESI-MS m/z 675 (M+H-56)⁺.

### Step 3. Synthesis of (3R)-3-(2-(3-chloro-5-fluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

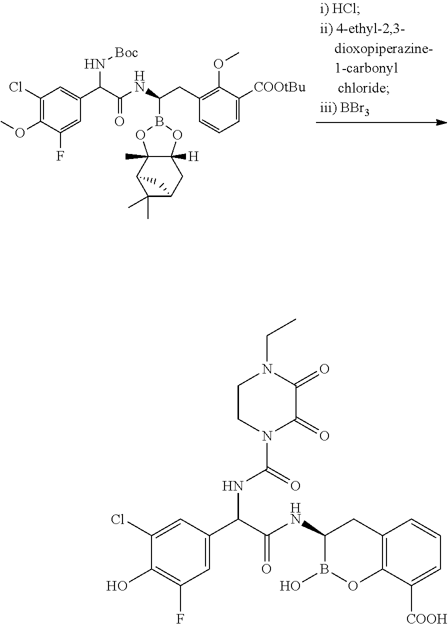

In a similar manner to the synthesis of Example 26, the title compound was prepared after reversed phase HPLC purification. ESI-MS m/z 578 (M+H)⁺.

### Example 165: (R)-3-((S)-2-(4-(2-aminoethyl)-2,3-dioxopiperazine-1-carboxamido)-2-(3,5-dichloro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(3,5-dichloro-4-methoxyphenyl)acetic acid

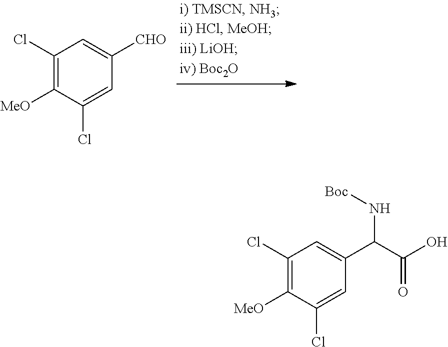 [SMILES: COc1c(Cl)cc(C=O)cc1Cl ; COc1c(Cl)cc(C(NC(=O)OC(C)(C)C)C(=O)O)cc1Cl]

In a same manner to the synthesis of Step 1 of Example 17, the title compound was prepared from 3-chloro-5-fluoro-4-methoxybenzaldehyde. ESI-MS m/z 373 (M+Na)⁺.

### Step 2. Synthesis of tert-butyl 3-((2R)-2-(2-((tert-butoxycarbonyl)amino)-2-(3,5-dichloro-4-hydroxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

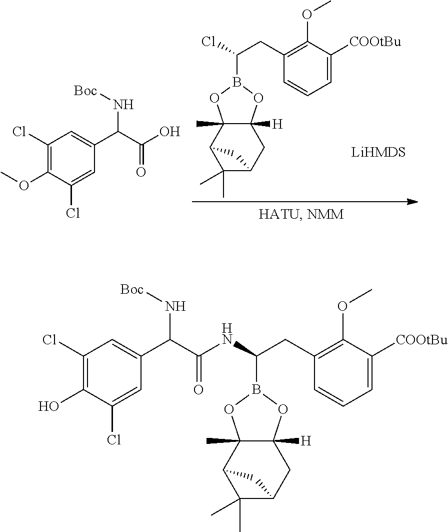

By following the General coupling method C, the title compound was prepared from the above acid and the chloride intermediate (prepared as previous reported, WO 2014/089365). ESI-MS m/z 692 (M+H-56)⁺.

### Step 3. Synthesis of tert-butyl (2-(4-(chlorocarbonyl)-2,3-dioxopiperazin-1-yl)ethyl)carbamate

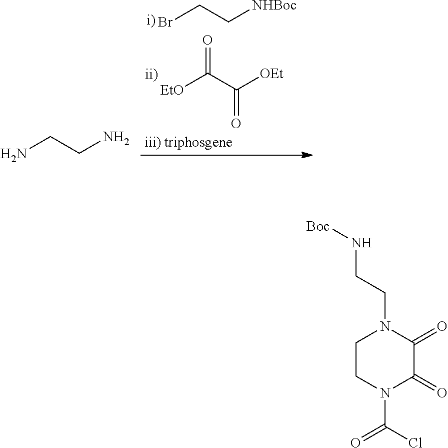

By following the same procedures as described for the synthesis of tert-butyl (2-(4-(chlorocarbonyl)-2,3-dioxopiperazin-1-yl)ethyl)carbamate in Step 1 of Example 26.

### Step 4. Synthesis of (R)-3-((S)-2-(4-(2-aminoethyl)-2,3-dioxopiperazine-1-carboxamido)-2-(3,5-dichloro-4-hydroxyphenyl)acetamido)-2-hydroxy-3,4-dihydro-2H-benzo[e][1,2]oxaborinine-8-carboxylic acid

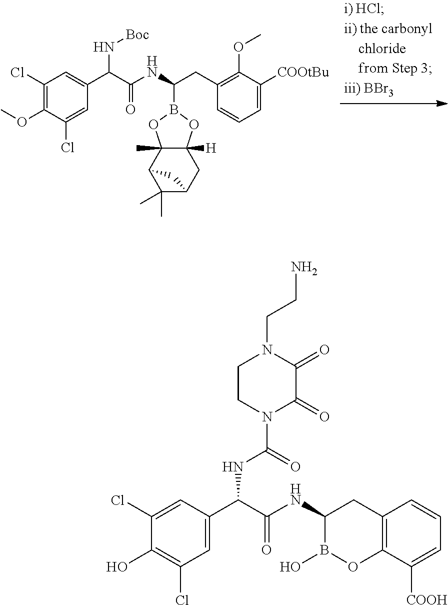

In a similar manner to the synthesis of Example 26, the title compound was prepared after reversed phase HPLC purification, isolated as the second eluting peak. ESI-MS m/z 609 (M+H)⁺.

### Example 166: (R)-3-((R)-2-(4-(2-aminoethyl)-2,3-dioxopiperazine-1-carboxamido)-2-(3,5-dichloro-4-hydroxyphenyl)acetamido)-2-hydroxy-3,4-dihydro-2H-benzo[e][1,2]oxaborinine-8-carboxylic acid

In a same manner to the synthesis of Example 166, the title compound was prepared after reversed phase HPLC purification. isolated as the first eluting peak. ESI-MS m/z 609 (M+H)⁺.

### Example 167: (R)-3-((S)-2-(4-(2-aminoethyl)-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2,3,5-trifluoro-4-methoxyphenyl)acetic acid

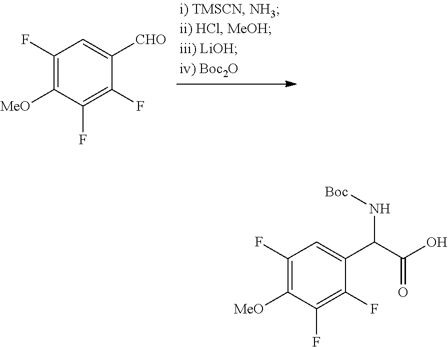 [SMILES: COc1c(F)cc(C=O)c(F)c1F ; COc1c(F)cc(C(NC(=O)OC(C)(C)C)C(=O)O)c(F)c1F]

In a same manner to the synthesis of Step 1 of Example 17, the title compound was prepared from 2,3,5-trifluoro-4-methoxybenzaldehyde. ESI-MS m/z 358 (M+Na)⁺.

### Step 2. Synthesis of tert-butyl 3-((2R)-2-(2-((tert-butoxycarbonyl)amino)-2-(2,3,5-trifluoro-4-hydroxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

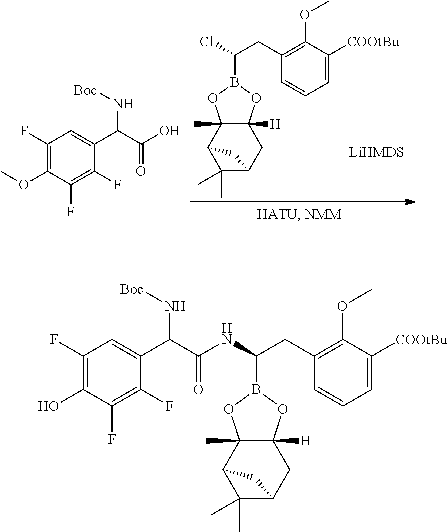

By following the General coupling method C, the title compound was prepared from the above acid and the chloride intermediate (prepared as previous reported, WO 2014/089365). ESI-MS m/z 676 (M+H-56)⁺.

### Step 3. Synthesis of tert-butyl (2-(4-(chlorocarbonyl)-2,3-dioxopiperazin-1-yl)ethyl)carbamate

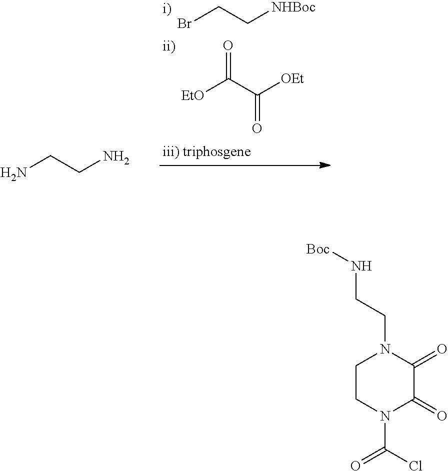

By following the same procedures as described for the synthesis of tert-butyl (2-(4-(chlorocarbonyl)-2,3-dioxopiperazin-1-yl)ethyl)carbamate in Step 1 of Example 26.

### Step 4. Synthesis of (R)-3-((S)-2-(4-(2-aminoethyl)-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

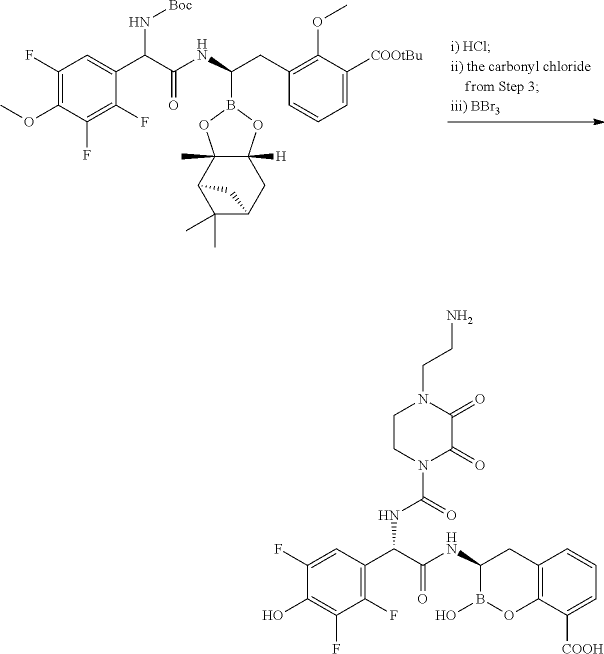

In a similar manner to the synthesis of Example 26, the title compound was prepared after reversed phase HPLC purification, isolated as the second eluting peak. ESI-MS m/z 594 (M+H)⁺.

### Example 168: (R)-3-((R)-2-(4-(2-aminoethyl)-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

In a same manner to the synthesis of Example 167, the title compound was prepared after reversed phase HPLC purification. isolated as the first eluting peak. ESI-MS m/z 594 (M+H)⁺.

### Example 169: (R)-3-((S)-2-(4-(2-aminoethyl)-2,3-dioxopiperazine-1-carboxamido)-2-(2,3-difluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2,3-difluoro-4-methoxyphenyl)acetic acid

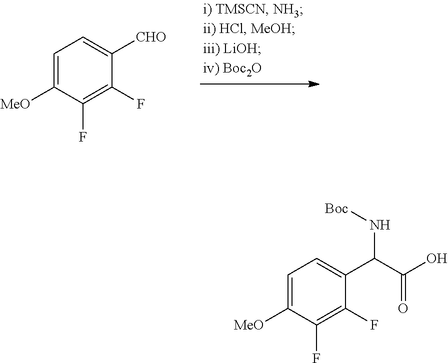 [SMILES: COc1ccc(C=O)c(F)c1F ; COc1ccc(C(NC(=O)OC(C)(C)C)C(=O)O)c(F)c1F]

In a same manner to the synthesis of Step 1 of Example 17, the title compound was prepared from 2,3-difluoro-4-methoxybenzaldehyde. ESI-MS m/z 340 (M+Na)⁺.

### Step 2. Synthesis of tert-butyl 3-((2R)-2-(2-((tert-butoxycarbonyl)amino)-2-(2,3-difluoro-4-hydroxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

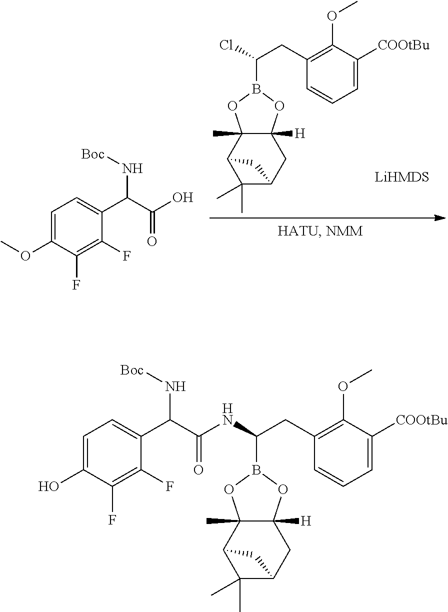

By following the General coupling method C, the title compound was prepared from the above acid and the chloride intermediate (prepared as previous reported, WO 2014/089365). ESI-MS m/z 659 (M+H-56)⁺.

### Step 3. Synthesis of tert-butyl (2-(4-(chlorocarbonyl)-2,3-dioxopiperazin-1-yl)ethyl)carbamate

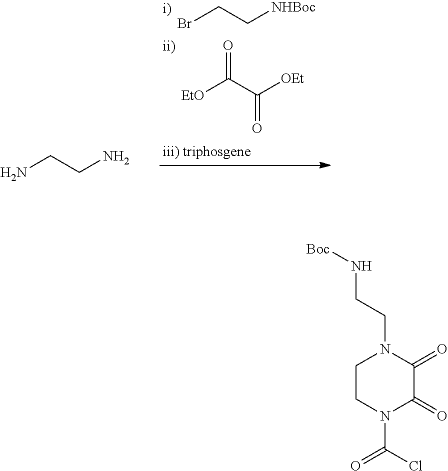

By following the same procedures as described for the synthesis of tert-butyl (2-(4-(chlorocarbonyl)-2,3-dioxopiperazin-1-yl)ethyl)carbamate in Step 1 of Example 26.

### Step 4. Synthesis of (R)-3-((S)-2-(4-(2-aminoethyl)-2,3-dioxopiperazine-1-carboxamido)-2-(2,3-difluoro-4-hydroxyphenyl)acetamido)-2-hydroxy-3,4-dihydro-2H-benzo[e][1,2]oxaborinine-8-carboxylic acid

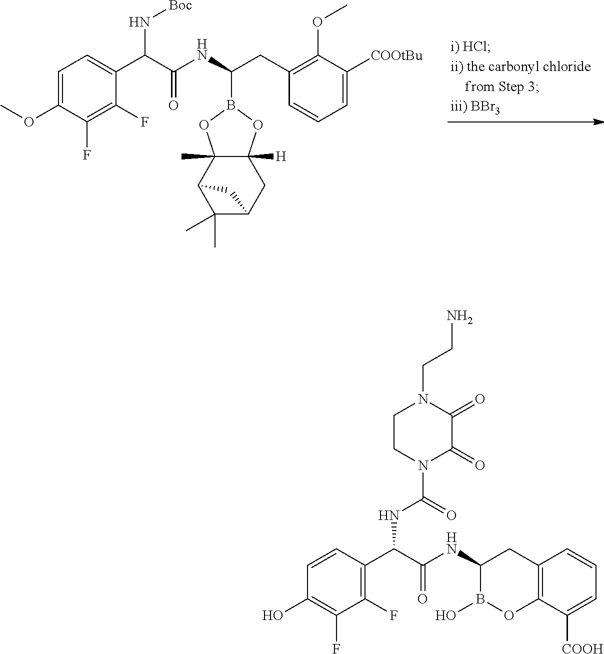

In a similar manner to the synthesis of Example 26, the title compound was prepared after reversed phase HPLC purification. isolated as the second eluting peak. ESI-MS m/z 576 (M+H)⁺.

### Example 170: (R)-3-((R)-2-(4-(2-aminoethyl)-2,3-dioxopiperazine-1-carboxamido)-2-(2,3-difluoro-4-hydroxyphenyl)acetamido)-2-hydroxy-3,4-dihydro-2H-benzo[e][1,2]oxaborinine-8-carboxylic acid

In a same manner to the synthesis of Example 170, the title compound was prepared after reversed phase HPLC purification. isolated as the first eluting peak. ESI-MS m/z 594 (M+H)⁺.

### Example 171: (3R)-3-(2-(3,5-dichloro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(3,5-dichloro-4-methoxyphenyl)acetic acid

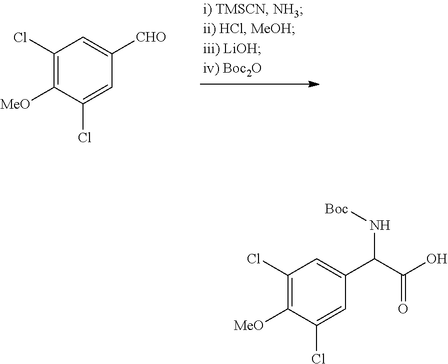 [SMILES: COc1c(Cl)cc(C=O)cc1Cl ; COc1c(Cl)cc(C(NC(=O)OC(C)(C)C)C(=O)O)cc1Cl]

In a same manner to the synthesis of Step 1 of Example 17, the title compound was prepared from 3,5-dichloro-4-methoxybenzaldehyde. ESI-MS m/z 373 (M+Na)⁺.

### Step 2. Synthesis of tert-butyl 3-((2R)-2-(2-((tert-butoxycarbonyl)amino)-2-(3,5-dichloro-4-hydroxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

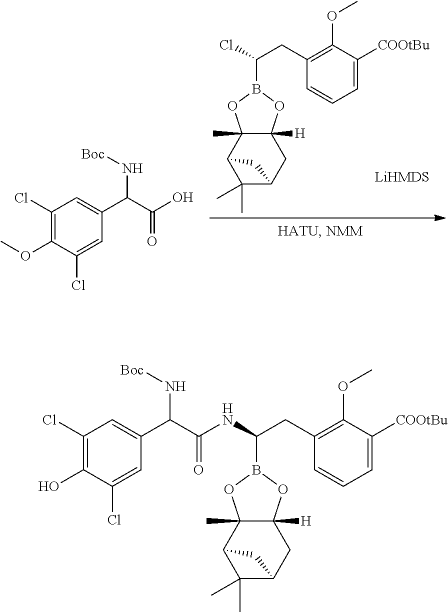

By following the General coupling method C, the title compound was prepared from the above acid and the chloride intermediate (prepared as previous reported, WO 2014/089365). ESI-MS m/z 692 (M+H-56)⁺.

### Step 3. Synthesis of (3R)-3-(2-(3,5-dichloro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

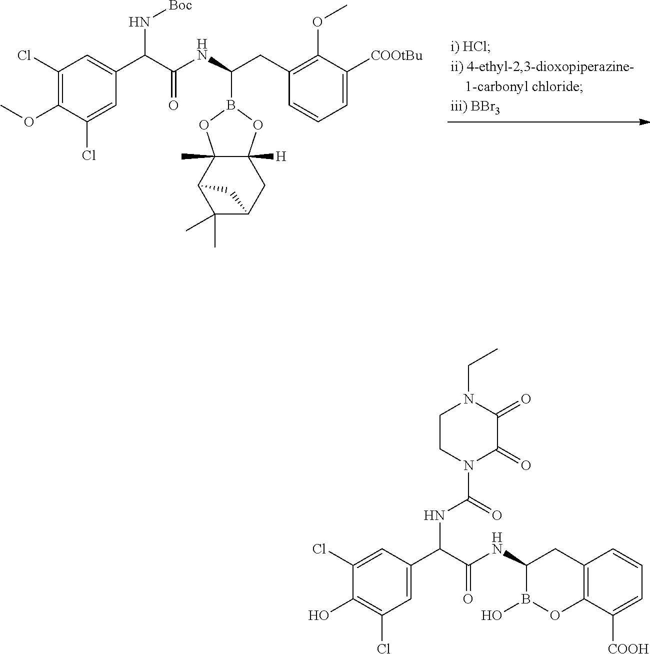

In a similar manner to the synthesis of Example 26, the title compound was prepared after reversed phase HPLC purification. ESI-MS m/z 594 (M+H)⁺.

### Example 172: (3R)-3-(2-(4-ethyl-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2,3,5-trifluoro-4-methoxyphenyl)acetic acid

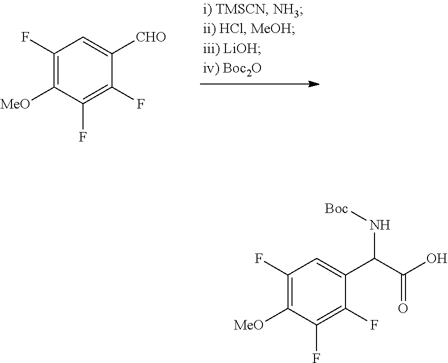 [SMILES: COc1c(F)cc(C=O)c(F)c1F ; COc1c(F)cc(C(NC(=O)OC(C)(C)C)C(=O)O)c(F)c1F]

In a same manner to the synthesis of Step 1 of Example 17, the title compound was prepared from 2,3,5-trifluoro-4-methoxybenzaldehyde. ESI-MS m/z 358 (M+Na)⁺.

### Step 2. Synthesis of tert-butyl 3-((2R)-2-(2-((tert-butoxycarbonyl)amino)-2-(2,3,5-trifluoro-4-hydroxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

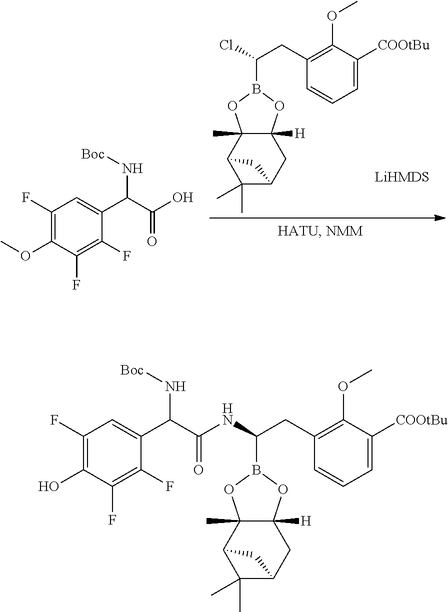

By following the General coupling method C, the title compound was prepared from the above acid and the chloride intermediate (prepared as previous reported, WO 2014/089365). ESI-MS m/z 676 (M+H-56)⁺.

### Step 3. Synthesis of (3R)-3-(2-(4-ethyl-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

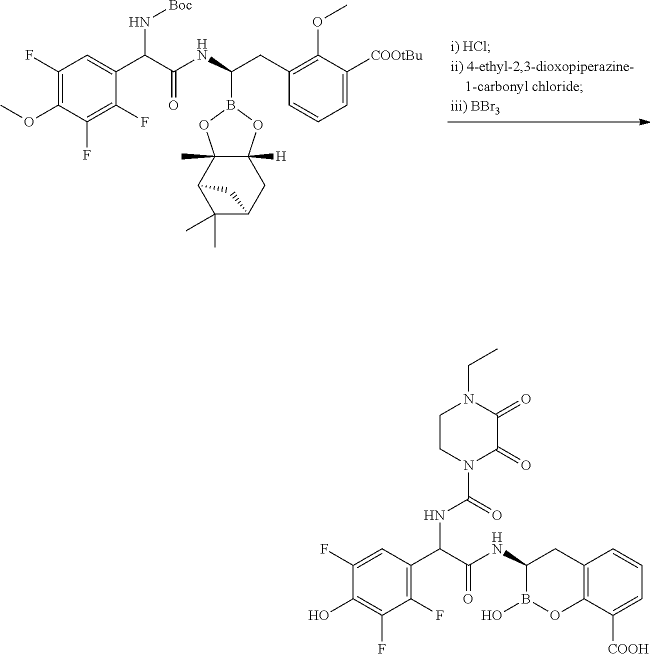

In a similar manner to the synthesis of Example 26, the title compound was prepared after reversed phase HPLC purification. ESI-MS m/z 579 (M+H)⁺.

### Example 174: (3R)-3-(2-(4-(2-aminoethyl)-2,3-dioxopiperazine-1-carboxamido)-2-(2,3,5,6-tetrafluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2,3,5,6-tetrafluoro-4-methoxyphenyl)acetic acid

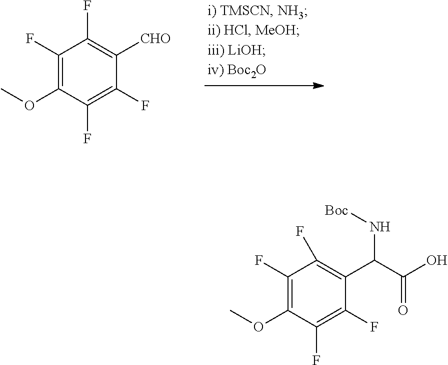 [SMILES: COc1c(F)c(F)c(C=O)c(F)c1F ; COc1c(F)c(F)c(C(NC(=O)OC(C)(C)C)C(=O)O)c(F)c1F]

The title compound was prepared in a similar manner to the synthesis step 1 of Example 17, utilizing 2,3,5,6-tetrafluoro-4-methoxybenzaldehyde in place of 4-methoxy-3-nitrobenzaldehyde. ESI-MS m/z 354.1 (M+H)⁺.

### Step 2: Synthesis of tert-butyl 3-((2R)-2-(2-((tert-butoxycarbonyl)amino)-2-(2,3,5,6-tetrafluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

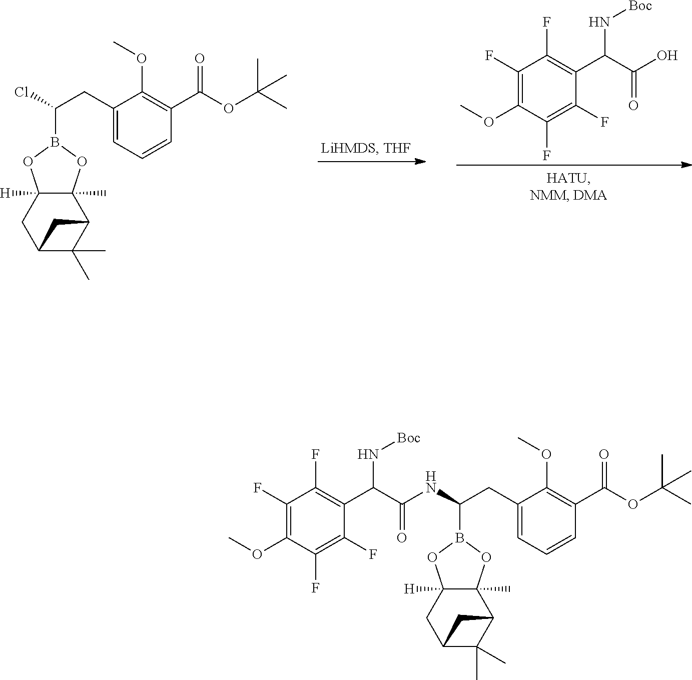 [SMILES: COc1c(C[C@@H](Cl)B2O[C@@H]3C[C@@H]4C[C@@H](C4(C)C)[C@]3(C)O2)cccc1C(=O)OC(C)(C)C ; COc1c(F)c(F)c(C(NC(=O)OC(C)(C)C)C(=O)O)c(F)c1F ; COc1c(C[C@H](NC(=O)C(NC(=O)OC(C)(C)C)c2c(F)c(F)c(OC)c(F)c2F)B2O[C@@H]3C[C@@H]4C[C@@H](C4(C)C)[C@]3(C)O2)cccc1C(=O)OC(C)(C)C]

By following the General Procedure C, the chloride (prepared as previous reported, WO 2014/089365) was treated with LiHMDS, and then coupled 2-((tert-butoxycarbonyl)amino)-2-(2,3,5,6-tetrafluoro-4-methoxyphenyl)acetic acid in the presence of HATU and NMM, yielding the title compound. ESI-MS m/z 765.3 (M+H)⁺.

### Step 3: Synthesis of (3R)-3-(2-(4-(2-aminoethyl)-2,3-dioxopiperazine-1-carboxamido)-2-(2,3,5,6-tetrafluoro-4-hydroxyphenyl)acetamido)-2-hydroxy-3,4-dihydro-2H-benzo[e][1,2]oxaborinine-8-carboxylic acid

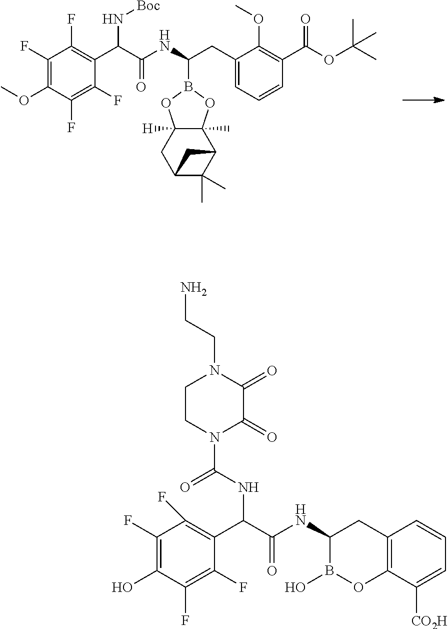 [SMILES: COc1c(C[C@H](NC(=O)C(NC(=O)OC(C)(C)C)c2c(F)c(F)c(OC)c(F)c2F)B2O[C@@H]3C[C@@H]4C[C@@H](C4(C)C)[C@]3(C)O2)cccc1C(=O)OC(C)(C)C ; NCCN1CCN(C(=O)NC(C(=O)N[C@H]2Cc3cccc(C(=O)O)c3OB2O)c2c(F)c(F)c(O)c(F)c2F)C(=O)C1=O]

The title compound was prepared in a similar manner to the synthesis of Example 1, utilizing tert-butyl (2-(4-(chlorocarbonyl)-2,3-dioxopiperazin-1-yl)ethyl)carbamate in place of tert-butyl (3-(4-(chlorocarbonyl)-2,3-dioxopiperazin-1-yl)propyl)carbamate. ESI-MS m/z 612.0 (M+H)⁺.

### Example 175: (3R)-3-(2-(4-ethyl-2,3-dioxopiperazine-1-carboxamido)-2-(2,3,5,6-tetrafluoro-4-hydroxyphenyl)acetamido)-2-hydroxy-3,4-dihydro-2H-benzo[e][1,2]oxaborinine-8-carboxylic acid

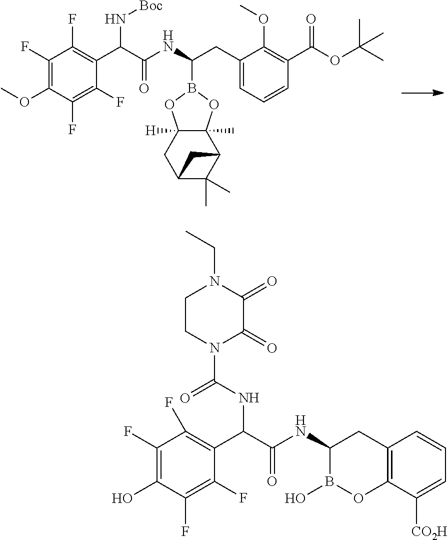

The title compound was prepared in a similar manner to the synthesis of Example 1, utilizing 4-ethyl-2,3-dioxopiperazine-1-carbonyl chloride in place of tert-butyl (3-(4-(chlorocarbonyl)-2,3-dioxopiperazin-1-yl)propyl)carbamate. ESI-MS m/z 597.2 (M+H)⁺.

### Example 176: (3R)-3-(2-(2,6-difluoro-4-(sulfamoylamino)phenyl)-2-(4-ethyl-2,3-dioxopiperazine-1-carboxamido)acetamido)-2-hydroxy-3,4-dihydro-2H-benzo[e][1,2]oxaborinine-8-carboxylic acid

By following the procedures of Step 1 and Step 2 of Example 17, utilizing benzyl (3,5-difluoro-4-formylphenyl)carbamate in place of 4-methoxy-3-nitrobenzaldehyde in Step 1, and then changing the reaction sequence as follows: i) hydrogenation to remove Cbz followed by acylation with benzyl (chlorosulfonyl)carbamate; ii) treated with HCl to remove Boc followed by acylation 4-ethyl-2,3-dioxopiperazine-1-carbonyl chloride; iii) treated with excess BBr₃. The title compound, Example 176 was prepared. ESI-MS m/z 639 (M+1)⁺.

### Example 177: (3R)-3-(2-(4-(2-aminoethyl)-2,3-dioxopiperazine-1-carboxamido)-2-(2,6-difluoro-4-(sulfamoylamino)phenyl)acetamido)-2-hydroxy-3,4-dihydro-2H-benzo[e][1,2]oxaborinine-8-carboxylic acid

In a similar manner to the synthesis of Example 176, utilizing tert-butyl (2-(4-(chlorocarbonyl)-2,3-dioxopiperazin-1-yl)ethyl)carbamate in place of 4-ethyl-2,3-dioxopiperazine-1-carbonyl chloride, the title compound was prepared. ESI-MS m/z 654 (M+1)⁺.

### Example 178: (3R)-3-(2-(4-(3-aminopropyl)-2,3-dioxopiperazine-1-carboxamido)-2-(2,6-difluoro-4-hydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 111, utilizing tert-butyl (3-(4-(chlorocarbonyl)-2,3-dioxopiperazin-1-yl)propyl)carbamate in place of tert-butyl (2-(4-(chlorocarbonyl)-2,3-dioxopiperazin-1-yl)ethyl)carbamate. ESI-MS m/z 608 (MH).

### Example 179: (R)-3-((R)-2-(4-(2-aminoethyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 42, utilizing tert-butyl (2-(4-(chlorocarbonyl)-2,3-dioxopiperazin-1-yl)ethyl)carbamate in place of 4-ethyl-2,3-dioxopiperazine-1-carbonyl chloride, the title compound was isolated as the first eluting peak after reversed phase HPLC purification. ESI-MS m/z 626/628 (MH/MH+2)⁺.

### Example 180: (R)-3-((R)-2-(4-(3-aminopropyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 42, utilizing tert-butyl (3-(4-(chlorocarbonyl)-2,3-dioxopiperazin-1-yl)propyl)carbamate in place of 4-ethyl-2,3-dioxopiperazine-1-carbonyl chloride, the title compound was isolated as the first eluting peak after reversed phase HPLC purification. ESI-MS m/z 640/642 (MH/MH+2)⁺.

### Example 181: (3R)-3-(2-(4-amino-2,6-difluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

By following the procedures of Step 1, Step 2, Step 3 and Step 4 of Example 17, utilizing benzyl (3,5-difluoro-4-formylphenyl)carbamate in place of 4-methoxy-3-nitrobenzaldehyde in Step 1, then the resulting product was treated with excess BBr₃, yielding the title compound. ESI-MS m/z 660 (M+1)⁺.

### Example 182: (R)-3-((R)-2-(4-ethyl-2,3-dioxopiperazine-1-carboxamido)-2-(2-fluoro-4,6-dihydroxyphenyl)acetamido)-2-hydroxy-3,4-dihydro-2H-benzo[e][1,2]oxaborinine-8-carboxylic acid

By following the procedures of Step 1, Step 2, Step 3 and Step 4 of Example 17, utilizing 2-fluoro-4,6-dimethoxybenzaldehyde in place of 4-methoxy-3-nitrobenzaldehyde in Step 1, then the resulting product was treated with excess BBr₃ at RT for 2 days, the title compound was isolated as the first eluting peak after reversed phase HPLC. ESI-MS m/z 659 (M+1)⁺.

### Example 183: (3R)-3-(2-(4-(3-aminopropyl)-2,3-dioxopiperazine-1-carboxamido)-2-(2,6-difluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 111, utilizing tert-butyl (3-(4-(chlorocarbonyl)-2,3-dioxopiperazin-1-yl)propyl)carbamate in place of tert-butyl (2-(4-(chlorocarbonyl)-2,3-dioxopiperazin-1-yl)ethyl)carbamate. ESI-MS m/z 590 (MH)⁺.

### Example 184: (3R)-3-(2-(4-ethyl-2,3-dioxopiperazine-1-carboxamido)-2-(2-fluoro-4-hydroxy-5-nitrophenyl)acetamido)-2-hydroxy-3,4-dihydro-2H-benzo[e][1,2]oxaborinine-8-carboxylic acid

The title compound was prepared in a similar manner to the synthesis of Example 105, utilizing 2-((tert-butoxycarbonyl)amino)-2-(2-fluoro-4-methoxy-5-nitrophenyl)acetic acid in place of 2-((tert-butoxycarbonyl)amino)-2-(2,6-difluoro-4-methoxyphenyl)acetic acid. ESI-MS m/z 588 (MH)⁺.

### Example 185: (3R)-3-(2-(3-chloro-2-fluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(3-chloro-2-fluoro-4-methoxyphenyl)acetic acid

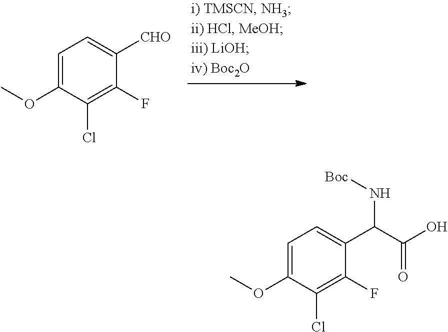 [SMILES: COc1ccc(C=O)c(F)c1Cl ; COc1ccc(C(NC(=O)OC(C)(C)C)C(=O)O)c(F)c1Cl]

The title compound was prepared in a similar manner to the synthesis step 1 of Example 17, utilizing 3-chloro-2-fluoro-4-methoxybenzaldehyde in place of 4-methoxy-3-nitrobenzaldehyde. ESI-MS m/z 334.1 (M+H)⁺.

### Step 2: Synthesis of tert-butyl 3-((2R)-2-(2-((tert-butoxycarbonyl)amino)-2-(3-chloro-2-fluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

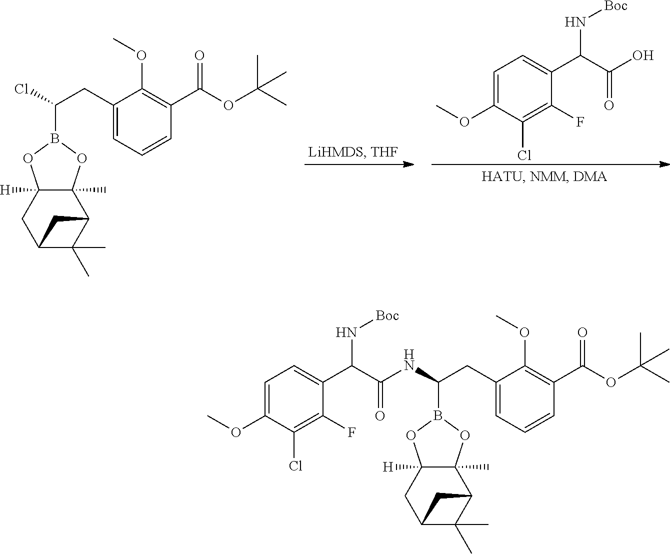 [SMILES: COc1c(C[C@@H](Cl)B2O[C@@H]3C[C@@H]4C[C@@H](C4(C)C)[C@]3(C)O2)cccc1C(=O)OC(C)(C)C ; COc1ccc(C(NC(=O)OC(C)(C)C)C(=O)O)c(F)c1Cl ; COc1ccc(C(NC(=O)OC(C)(C)C)C(=O)N[C@@H](Cc2cccc(C(=O)OC(C)(C)C)c2OC)B2O[C@@H]3C[C@@H]4C[C@@H](C4(C)C)[C@]3(C)O2)c(F)c1Cl]

By following the General Procedure C, the chloride (prepared as previous reported, WO 2014/089365) was treated with LiHMDS, and then coupled 2-((tert-butoxycarbonyl)amino)-2-(3-chloro-2-fluoro-4-methoxyphenyl)acetic acid in the presence of HATU and NMM, yielding the title compound. ESI-MS m/z 745.3 (M+H)⁺.

### Step 3: Synthesis of (3R)-3-(2-(3-chloro-2-fluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 1, utilizing 4-ethyl-2,3-dioxopiperazine-1-carbonyl chloride in place of tert-butyl (3-(4-(chlorocarbonyl)-2,3-dioxopiperazin-1-yl)propyl)carbamate. ESI-MS m/z 577.2 (M+H)⁺.

### Example 186: (R)-3-((R)-2-(4-(2-aminoethyl)-2,3-dioxopiperazine-1-carboxamido)-2-(3-chloro-2-fluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 1, utilizing tert-butyl (2-(4-(chlorocarbonyl)-2,3-dioxopiperazin-1-yl)ethyl)carbamate in place of tert-butyl (3-(4-(chlorocarbonyl)-2,3-dioxopiperazin-1-yl)propyl)carbamate, isolated as the first eluting peak. ESI-MS m/z 591.9 (M+H)⁺.

### Example 187: (R)-3-((S)-2-(4-(2-aminoethyl)-2,3-dioxopiperazine-1-carboxamido)-2-(3-chloro-2-fluoro-4-hydroxyphenyl)acetamido)-2-hydroxy-3,4-dihydro-2H-benzo[e][1,2]oxaborinine-8-carboxylic acid

The title compound was prepared in a same manner to the synthesis of Example 186, via the purification of reverse phase HPLC, isolated as the second eluting peak. ESI-MS m/z 591.9 (M+H)⁺.

### Example 188: (3R)-3-(2-(4-acetamido-2,6-difluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1: Synthesis of tert-butyl 3-((2R)-2-(2-(4-acetamido-2,6-difluorophenyl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

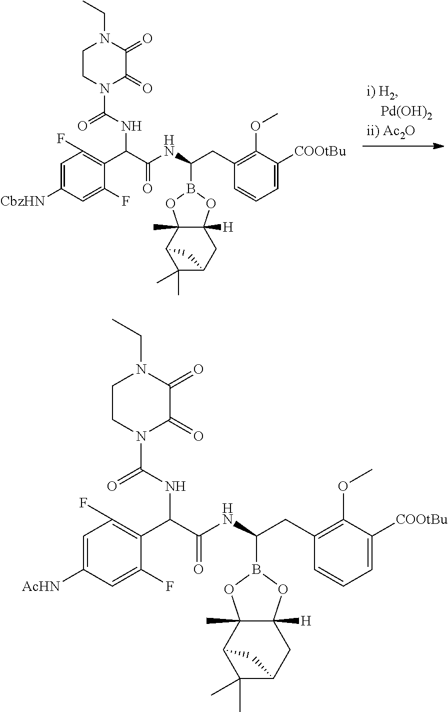

To tert-butyl 3-((2R)-2-(2-(4-(((benzyloxy)carbonyl)amino)-2,6-difluorophenyl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate 1.2 g (1.31 mmol) in methanol (15 mL) under an atmosphere of argon was added a spatula tip of palladium hydroxide on carbon and the reaction mixture was stirred under 1 atm hydrogen balloon for 18 h and filtered through a pad of celite. The filtrate was concentrated in vacuo to give tert-butyl 3-((2R)-2-(2-(4-amino-2,6-difluorophenyl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate, 0.84 g, (82%). ESI-MS m/z 782 (MH)⁺. To this intermediate 0.42 g (0.54 mmol) in dichloromethane (5 mL) at 0° C. was added triethylamine 0.14 mL (1.02 mmol, 1.9 eq), followed by acetyl chloride 0.04 mL (0.51 mmol, 0.95 eq) and warmed at RT. After 1 h, excess acetyl chloride was added until all starting material was consumed by LCMS. The reaction was washed with water, dried over sodium sulfate, and concentrated to give the title compound, ESI-MS m/z 824 (MH)⁺.

### Step 2: Synthesis of (3R)-3-(2-(4-acetamido-2,6-difluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

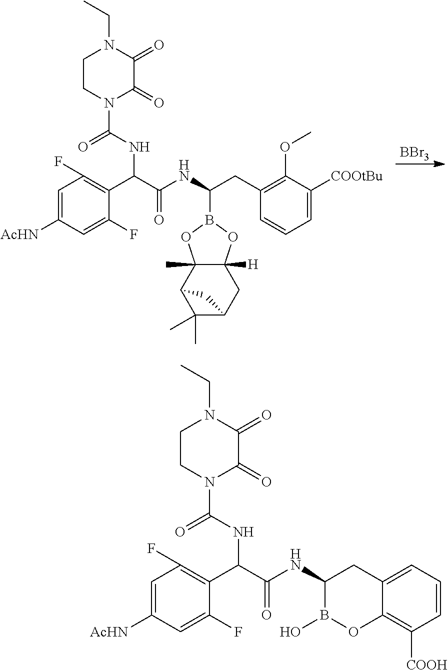

By following the general deprotection and purification method, the above compound was treated with excess BBr₃ to yield the title compound after reversed phase HPLC purification. ESI-MS m/z 602 (MH)⁺.

### Example 189: (R)-3-((R)-2-(2-chloro-5-fluoro-3,4-dihydroxyphenyl)-2-(3-(methylsulfonyl)-2-oxoimidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 42, utilizing 3-(methylsulfonyl)-2-oxoimidazolidine-1-carbonyl chloride in place of 4-ethyl-2,3-dioxopiperazine-1-carbonyl chloride, the title compound was isolated as the second eluting peak after reversed phase HPLC purification. ESI-MS m/z 633/635 (MH/MH+2)⁺.

### Example 190: (R)-3-((R)-2-(4-(3-aminopropyl)-2,3-dioxopiperazine-1-carboxamido)-2-(2-chloro-6-fluoro-3,4-dihydroxyphenyl)acetamido)-6-chloro-7-fluoro-2-hydroxy-3,4-dihydro-2H-benzo[e][1,2]oxaborinine-8-carboxylic acid

Example 27 (TFA salt, 36 mg, 0.048 mmol) was treated with NCS (12 mg, 0.09 mmol) in DMF (1 mL) at RT for 1 h, diluted with 1 mL of water, purified by reverse phase HPLC to yield the title compound, 9 mg. ESI-MS m/z 674/676/678 (MH/MH+2/MH+4)⁺.

### Example 191: (R)-6-chloro-3-((R)-2-(2-chloro-6-fluoro-3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of (R)-3-((R)-2-(2-chloro-6-fluoro-3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

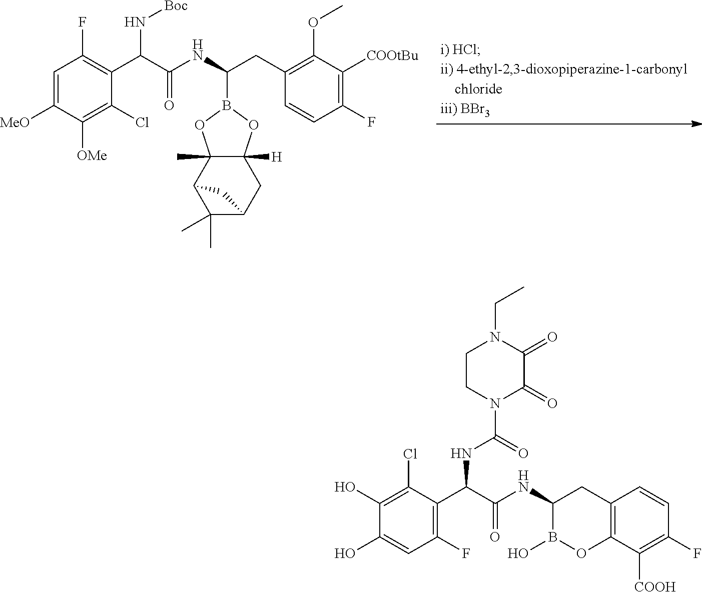

In a similar manner to the synthesis of Example 26, following the procedures of step 2, utilizing 4-ethyl-2,3-dioxopiperazine-1-carbonyl chloride in place of tert-butyl (2-(4-(chlorocarbonyl)-2,3-dioxopiperazin-1-yl)ethyl)carbamatethe, the title compound was isolated as the first eluting peak after reversed phase HPLC purification. ESI-MS m/z 611/613 (MH/MH+2)⁺.

### Step 2. Synthesis of (R)-6-chloro-3-((R)-2-(2-chloro-6-fluoro-3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

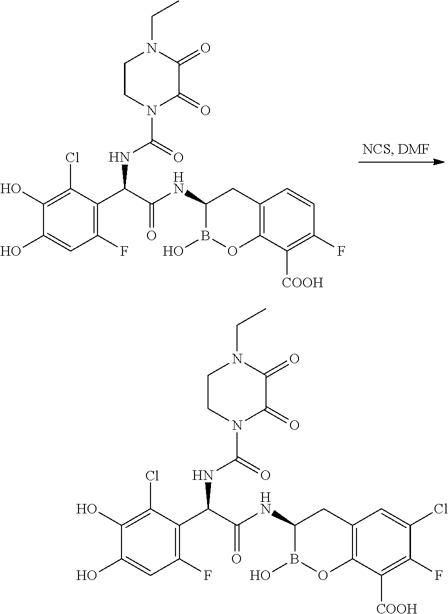

The above product (36 mg, 0.059 mmol) was treated with NCS (13.4 mg, 1 mmol) in DMF (1 mL) at RT for 50 min, diluted with 0.8 mL of water, purified by reverse phase HPLC to yield the title compound, 8 mg. ESI-MS m/z 645/647/649 (MH/MH+2/MH+4)⁺.

### Example 192: (3R)-3-(2-(5-cyano-2-fluoro-4-methoxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1: Synthesis of methyl 2-((tert-butoxycarbonyl)amino)-2-(5-cyano-2-fluoro-4-methoxyphenyl)acetate

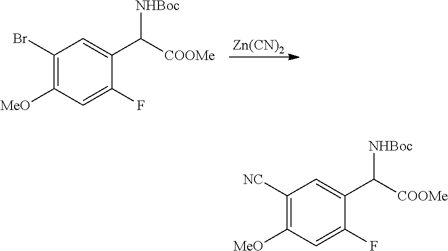

To methyl 2-(5-bromo-2-fluoro-4-methoxyphenyl)-2-((tert-butoxycarbonyl)amino)acetate 0.78 g (1.98 mmol) was added zinc cyanide 0.47 g (3.92 mmol, 2 eq), tetrakis(triphenylphosphine)palladium(0) 0.23 g (0.19 mmol) 10 mol %), followed by 1-methyl-2-pyrrolidinone (62 mL) and degassed 3× under argon. The mixture was stirred at 140° C. for 2 h, cooled, diluted with ethyl acetate, washed with water 3×, dried over sodium sulfate, and concentrated. The crude product was purified by silica gel chromatography (30% ethyl acetate/hexanes) to give the title compound, 0.563 g, (84%). ESI-MS m/z 339 (MH)⁺.

### Step 2: Synthesis of tert-butyl 3-((2R)-2-(2-((tert-butoxycarbonyl)amino)-2-(5-cyano-2-fluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

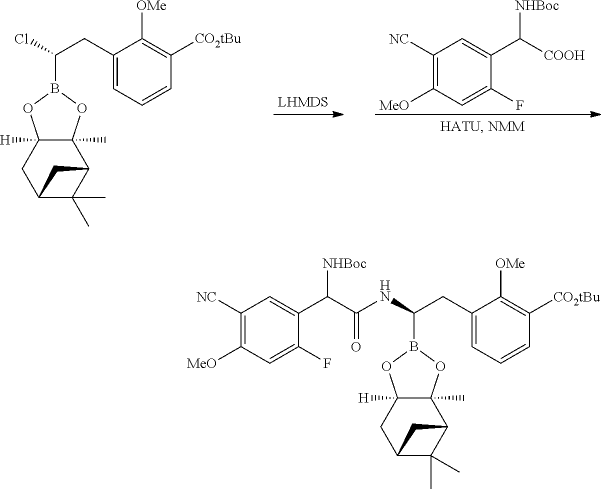 [SMILES: COc1c(C[C@@H](Cl)B2O[C@@H]3C[C@@H]4C[C@@H](C4(C)C)[C@]3(C)O2)cccc1C(=O)OC(C)(C)C ; COc1cc(F)c(C(NC(=O)OC(C)(C)C)C(=O)O)cc1C#N ; COc1cc(F)c(C(NC(=O)OC(C)(C)C)C(=O)N[C@@H](Cc2cccc(C(=O)OC(C)(C)C)c2OC)B2O[C@@H]3C[C@@H]4C[C@@H](C4(C)C)[C@]3(C)O2)cc1C#N]

By following the General Procedure C, the chloride (prepared as previous reported, WO 2014/089365) was treated with LiHMDS, and then coupled 2-((tert-butoxycarbonyl)amino)-2-(5-cyano-2-fluoro-4-methoxyphenyl)acetic acid in the presence of HATU and NMM, yielding the title compound. ESI-MS m/z 736 (MH)⁺.

### Step 3: Synthesis of (3R)-3-(2-(5-cyano-2-fluoro-4-methoxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

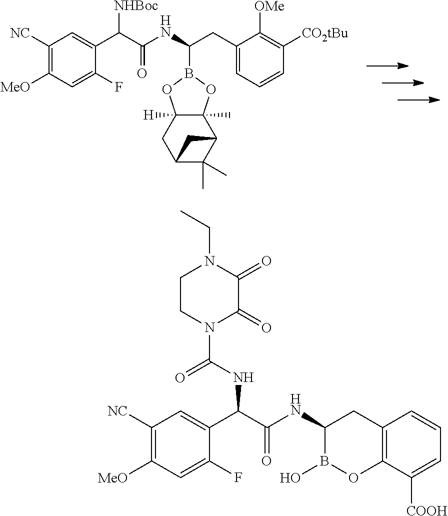

By following the general deprotection and purification method, the above compound was treated with excess BBr₃ and stirred at RT for 18 h to yield the title compound after reversed phase HPLC purification. ESI-MS m/z 582 (MH)⁺.

### Example 193: (3R)-3-(2-(4-(azetidin-3-ylmethyl)-2,3-dioxopiperazine-1-carboxamido)-2-(2,6-difluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 111, utilizing tert-butyl 3-((4-(chlorocarbonyl)-2,3-dioxopiperazin-1-yl)methyl)azetidine-1-carboxylate in place of tert-butyl (2-(4-(chlorocarbonyl)-2,3-dioxopiperazin-1-yl)ethyl)carbamate. ESI-MS m/z 602 (MH)⁺.

### Example 194: (R)-3-((R)-2-(4-(2-aminoethyl)-2,3-dioxopiperazine-1-carboxamido)-2-(2-fluoro-4,6-dihydroxyphenyl)acetamido)-2-hydroxy-3,4-dihydro-2H-benzo[e][1,2]oxaborinine-8-carboxylic acid

In a similar manner to the synthesis of Example 182, utilizing tert-butyl (2-(4-(chlorocarbonyl)-2,3-dioxopiperazin-1-yl)ethyl)carbamatethe in place of 4-ethyl-2,3-dioxopiperazine-1-carbonyl chloride, the title compound was isolated as the first eluting peak after reversed phase HPLC purification. ESI-MS m/z 574 (M+1)⁺.

### Example 195: (R)-3-((R)-2-(4-(3-aminopropyl)-2,3-dioxopiperazine-1-carboxamido)-2-(2-fluoro-4,6-dihydroxyphenyl)acetamido)-2-hydroxy-3,4-dihydro-2H-benzo[e][1,2]oxaborinine-8-carboxylic acid

In a similar manner to the synthesis of Example 182, utilizing tert-butyl (2-(4-(chlorocarbonyl)-2,3-dioxopiperazin-1-yl)propyl)carbamatethe in place of 4-ethyl-2,3-dioxopiperazine-1-carbonyl chloride, the title compound was isolated as the first eluting peak after reversed phase HPLC purification. ESI-MS m/z 588 (M+1)⁺.

### Example 196: (3R)-3-(2-(4-carboxy-3-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-(4-(tert-butoxycarbonyl)-3-methoxyphenyl)-2-((tert-butoxycarbonyl)amino)acetic acid in place of 2-(4-(tert-butoxycarbonyl)-2,6-difluorophenyl)-2-((tert-butoxycarbonyl)amino)acetic acid. ESI-MS m/z 569 (MH)⁺.

### Example 197: (3R)-3-(2-(4-ethyl-2,3-dioxopiperazine-1-carboxamido)-2-(2-fluoro-5-(sulfamoylamino)phenyl)acetamido)-2-hydroxy-3,4-dihydro-2H-benzo[e][1,2]oxaborinine-8-carboxylic acid

By following the procedures of Step 1 and Step 2 of Example 17, utilizing benzyl (4-fluoro-3-formylphenyl)carbamate in place of 4-methoxy-3-nitrobenzaldehyde in Step 1, and then changing the reaction sequence as follows: i) hydrogenation to remove Cbz followed by acylation with benzyl (chlorosulfonyl)carbamate; ii) treated with HCl to remove Boc followed by acylation 4-ethyl-2,3-dioxopiperazine-1-carbonyl chloride; iii) treated with excess BBr₃. The title compound was prepared. ESI-MS m/z 621 (M+1)⁺.

### Example 198: (3R)-3-(2-(2-fluoro-5-(sulfamoylamino)phenyl)-2-(4-(2-fluoroethyl)-2,3-dioxopiperazine-1-carboxamido)acetamido)-2-hydroxy-3,4-dihydro-2H-benzo[e][1,2]oxaborinine-8-carboxylic acid

In a similar manner to the synthesis of Example 197, utilizing 4-(2-fluoroethyl)-2,3-dioxopiperazine-1-carbonyl chloride in place of 4-ethyl-2,3-dioxopiperazine-1-carbonyl chloride, the title compound was prepared after reversed phase HPLC purification. ESI-MS m/z 639 (M+1)⁺.

### Example 199: (R)-3-((R)-2-(4-(2-aminoethyl)-2,3-dioxopiperazine-1-carboxamido)-2-(4-hydroxy-3-nitrophenyl)acetamido)-2-hydroxy-3,4-dihydro-2H-benzo[e][1,2]oxaborinine-8-carboxylic acid

The title compound was prepared in a similar manner to the synthesis of Example 17, utilizing 2-((tert-butoxycarbonyl)amino)-2-(4-methoxy-3-nitrophenyl)acetic acid in place of 2-((tert-butoxycarbonyl)amino)-2-(4-methoxy-3-nitrophenyl)acetic acid, isolated as the first eluting peak. ESI-MS m/z 585 (MH)⁺.

### Example 200: (R)-3-((S)-2-(4-(2-aminoethyl)-2,3-dioxopiperazine-1-carboxamido)-2-(4-hydroxy-3-nitrophenyl)acetamido)-2-hydroxy-3,4-dihydro-2H-benzo[e][1,2]oxaborinine-8-carboxylic acid

The title compound was prepared in a similar manner to the synthesis of Example 199, the title compound was prepared after reversed phase HPLC purification, isolated as the second eluting peak. ESI-MS m/z 585 (MH)⁺.

### Example 201: (3R)-3-(2-(4-carboxy-2,3-dichlorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 17, utilizing 2-(4-(tert-butoxycarbonyl)-2,3-dichlorophenyl)-2-((tert-butoxycarbonyl)amino)acetic acid in step 2, utilizing tert-butyl 4-(2-(((R)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-((tert-butoxycarbonyl)amino)-2-oxoethyl)-2,3-dichlorobenzoate in step 3, utilizing tert-butyl 4-(1-amino-2-(((R)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-2-oxoethyl)-2,3-dichlorobenzoate in step 4, utilizing tert-butyl 4-(2-(((R)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-(4-ethyl-2,3-dioxopiperazine-1-carboxamido)-2-oxoethyl)-2,3-dichlorobenzoate in step 6, the title compound was prepared. ESI-MS m/z 620.7 (MH)⁺.

### Example 202: (R)-3-((S)-2-(4-carboxy-2-chloro-3-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

### Step 1. Synthesis of 2-(4-(tert-butoxycarbonyl)-2-chloro-3-fluorophenyl)-2-((tert-butoxycarbonyl)amino)acetic acid

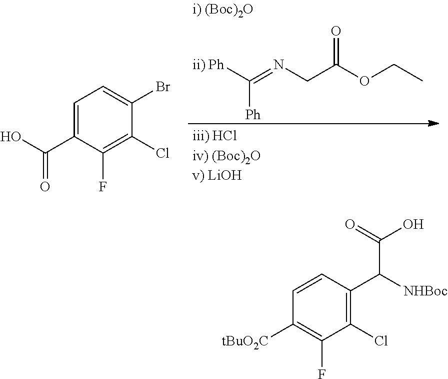

In a same manner to the synthesis of Step 1, 2, 3, 4, 5 of Example 157, the title compound was prepared from 4-bromo-3-chloro-2-fluorobenzoic acid. ESI-MS m/z 427 (M+Na)⁺.

### Step 2. Synthesis of tert-butyl 4-(2-(((R)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-((tert-butoxycarbonyl)amino)-2-oxoethyl)-3-chloro-2-fluorobenzoate

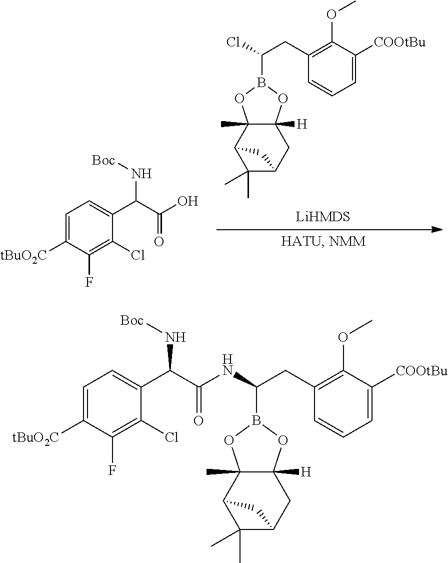

By following the General coupling method C, the title compound was prepared from the above acid and the chloride intermediate (prepared as previous reported, WO 2014/089365). ESI-MS m/z 760 (M-56+H)⁺.

### Step 3. Synthesis of (R)-3-((S)-2-(4-carboxy-2-chloro-3-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

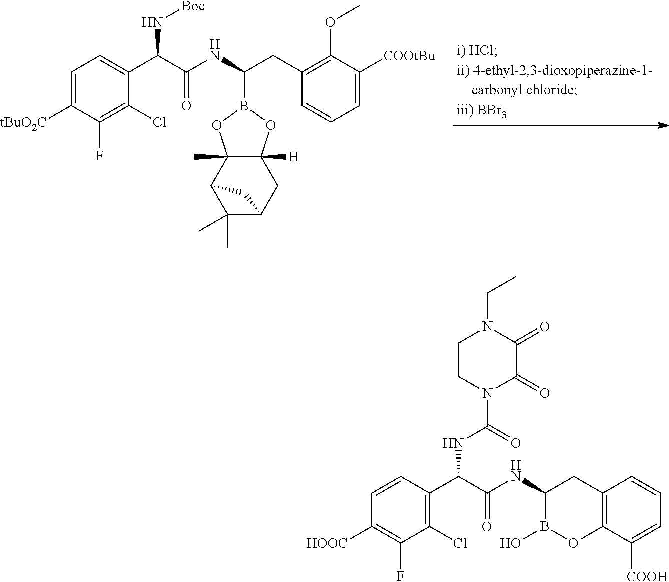

In a same manner to the synthesis of Example 148, the title compound was prepared after reversed phase HPLC purification, isolated as the first eluting peak. ESI-MS m/z 605 (M+H)⁺.

### Example 203: (R)-3-((R)-2-(4-carboxy-2-chloro-3-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

In a same manner to the synthesis of Example 86, the title compound was prepared after reversed phase HPLC purification, isolated as the second eluting peak. ESI-MS m/z 605 (M+H)⁺.

### Example 204: (R)-3-((S)-2-(4-(2-aminoethyl)-2,3-dioxopiperazine-1-carboxamido)-2-(2,6-difluorophenyl)acetamido)-2-hydroxy-3,4-dihydro-2H-benzo[e][1,2]oxaborinine-8-carboxylic acid

The title compound was prepared in a similar manner to the synthesis of Example 17, utilizing 2-((tert-butoxycarbonyl)amino)-2-(2,6-difluorophenyl)acetic acid in place of 2-((tert-butoxycarbonyl)amino)-2-(4-methoxy-3-nitrophenyl)acetic acid, isolated as the second eluting peak. ESI-MS m/z 560 (1MH)⁺.

### Example 205: (R)-3-((R)-2-(4-(2-aminoethyl)-2,3-dioxopiperazine-1-carboxamido)-2-(2-chloro-6-fluorophenyl)acetamido)-2-hydroxy-3,4-dihydro-2H-benzo[e][1,2]oxaborinine-8-carboxylic acid

The title compound was prepared in a similar manner to the synthesis of Example 17, utilizing 2-((tert-butoxycarbonyl)amino)-2-(2-chloro-6-fluorophenyl)acetic acid in place of 2-((tert-butoxycarbonyl)amino)-2-(4-methoxy-3-nitrophenyl)acetic acid, isolated as the first eluting peak. ESI-MS m/z 576 (MH)⁺.

### Example 206: (R)-3-((S)-2-(4-(2-aminoethyl)-2,3-dioxopiperazine-1-carboxamido)-2-(2-chloro-6-fluorophenyl)acetamido)-2-hydroxy-3,4-dihydro-2H-benzo[e][1,2]oxaborinine-8-carboxylic acid

The title compound was prepared in a similar manner to the synthesis of Example 205, the title compound was prepared after reversed phase HPLC purification, isolated as the second eluting peak. ESI-MS m/z 576 (MH)⁺.

### Example 207: (R)-3-((R)-2-(6-chloro-2-fluoro-3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 42, utilizing 2-fluoro-3-hydroxy-4-methoxybenzaldehyde in place of 3-fluoro-4-methoxy-5-hydroxybenzaldehyde in Step 1, the title compound was isolated as the first eluting peak after reversed phase HPLC purification. ESI-MS m/z 611/613 (MH/MH+2)⁺.

### Example 208: (3R)-3-(2-(6-chloro-2-fluoro-3,4-dihydroxyphenyl)-2-(3-(methylsulfonyl)-2-oxoimidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 207, utilizing 3-(methylsulfonyl)-2-oxoimidazolidine-1-carbonyl chloride in place of 4-ethyl-2,3-dioxopiperazine-1-carbonyl chloride, the title compound was obtained after reversed phase HPLC purification. ESI-MS m/z 633/635 (MH/MH+2)⁺.

### Example 209: (3R)-3-(2-(5-cyano-2-fluoro-4-hydroxyphenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 192, utilizing 2-(4-(benzyloxy)-5-cyano-2-fluorophenyl)-2-((tert-butoxycarbonyl)amino)acetic acid in place of 2-((tert-butoxycarbonyl)amino)-2-(5-cyano-2-fluoro-4-methoxyphenyl)acetic acid. ESI-MS m/z 568 (MH)⁺.

### Example 210: (R)-3-((S)-2-(3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

### Example 211: (R)-3-((R)-2-(3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of (2-chloro-3,4-dimethoxyphenyl)(cyano)methyl ethyl carbonate

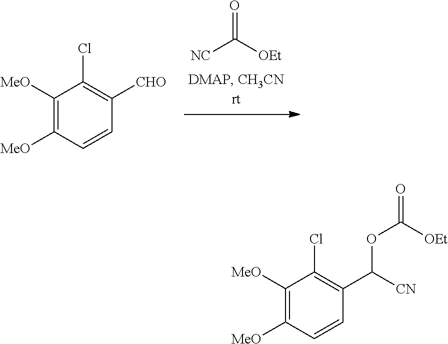 [SMILES: COc1ccc(C=O)c(Cl)c1OC ; CCOC(=O)C#N ; CCOC(=O)OC(C#N)c1ccc(OC)c(OC)c1Cl]

To a mixture of 2-chloro-3,4-dimethoxybenzaldehyde (30.30 g, 151 mmol) and ethyl cyanoformate (17.49 g, 176 mmol) in acetonitrile (300 mL) was added DMAP (1.87 g, 15.3 mmol) at room temperature. The reaction mixture was stirred at room temperature for 16 h. After the solvent was evaporated under reduced pressure, the residue was purified by flash chromatography on silica gel (350 g column) eluted with 0 to 40% ethyl acetate/hexanes to afford 43.23 g (95.5%) of (2-chloro-3,4-dimethoxyphenyl)(cyano)methyl ethyl carbonate as a solid. ESI-MS m/z 321.7, 323.8 (M+Na)⁺, 209.9, 211.9 (MH⁺-OCO₂Et).

### Step 2. Synthesis of ethyl 2-(2-chloro-3,4-dimethoxyphenyl)-2-oxoacetate

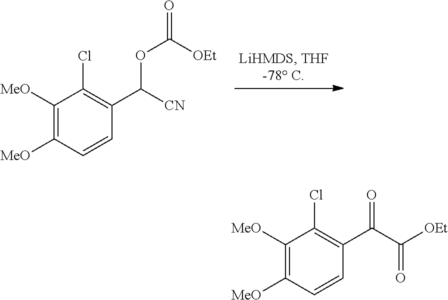 [SMILES: CCOC(=O)OC(C#N)c1ccc(OC)c(OC)c1Cl ; CCOC(=O)C(=O)c1ccc(OC)c(OC)c1Cl]

To a solution of (2-chloro-3,4-dimethoxyphenyl)(cyano)methyl ethyl carbonate (41.68 g, 139 mmol)) in THF (300 mL) at −78° C. under argon was added LiHMDS (1.0 M in THF, 230 mL, 230 mmol) dropwise. The reaction mixture was stirred at −78° C. for 5 h and then quenched with saturated NH₄Cl, extracted with ethyl acetate (3×). The combined organic phase was dried over Na₂SO₄. After the solvent was evaporated under reduced pressure, the residue was purified by flash chromatography on silica gel (350 g column) eluted with 0 to 50% ethyl acetate/hexanes to afford 33.80 g (89%) of ethyl 2-(2-chloro-3,4-dimethoxyphenyl)-2-oxoacetate as an oil. ESI-MS m/z 272.8 (M+H)⁺, 294.8 (M+Na)⁺, 566.5 (2M+Na)⁺.

### Step 3. Synthesis of ethyl (E)-2-((benzyloxy)imino)-2-(2-chloro-3,4-dimethoxyphenyl)acetate and ethyl (Z)-2-((benzyloxy)imino)-2-(2-chloro-3,4-dimethoxyphenyl)acetate

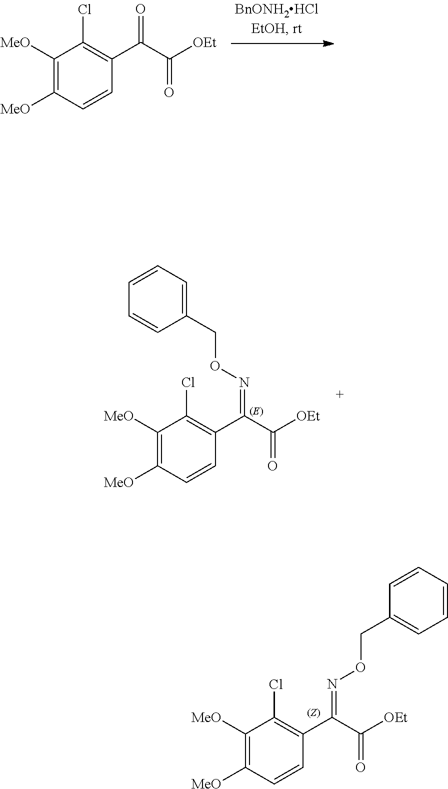 [SMILES: CCOC(=O)C(=O)c1ccc(OC)c(OC)c1Cl ; CCOC(=O)/C(=N/OCc1ccccc1)c1ccc(OC)c(OC)c1Cl ; CCOC(=O)/C(=N\OCc1ccccc1)c1ccc(OC)c(OC)c1Cl]

To a solution of ethyl 2-(2-chloro-3,4-dimethoxyphenyl)-2-oxoacetate (23.737 g, 87.0 mmol) in EtOH (350 mL) was added O-benzylhydroxylamine hydrochloride (25.39 g, 159 mmol) at room temperature. The reaction mixture was stirred at room temperature for 18 h and then quenched with saturated brine, extracted with ethyl acetate (3×). The combined organic phase was dried over Na₂SO₄. After the solvent was evaporated under reduced pressure, the residue was purified by flash chromatography on silica gel (350 g column) eluted with 0 to 50% ethyl acetate/hexanes to afford 6.90 g (21.0%) of ethyl (E)-2-((benzyloxy)imino)-2-(2-chloro-3,4-dimethoxyphenyl)acetate and 19.92 g (60.6%) of ethyl (Z)-2-((benzyloxy)imino)-2-(2-chloro-3,4-dimethoxyphenyl)acetate. ESI-MS m/z 377.7 (M+H)⁺, 399.7 (M+Na)⁺.

### Step 4. Synthesis of (E)-2-((benzyloxy)imino)-2-(2-chloro-3,4-dimethoxyphenyl)acetic acid

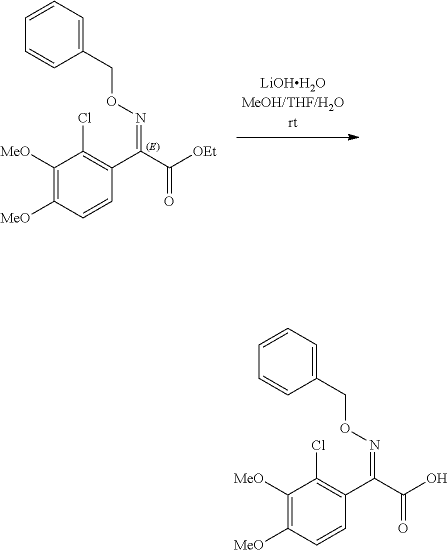 [SMILES: CCOC(=O)/C(=N/OCc1ccccc1)c1ccc(OC)c(OC)c1Cl ; COc1ccc(/C(=N\OCc2ccccc2)C(=O)O)c(Cl)c1OC]

A mixture of ethyl (E)-2-((benzyloxy)imino)-2-(2-chloro-3,4-dimethoxyphenyl)acetate (5.580 g, 14.77 mmol), lithium hydroxide monohydrate (2.843 g, 67.75 mmol) in THF (50 mL), MeOH (50 mL), and water (40 mL) was vigorously stirred at room temperature for 18 h. The reaction mixture was then quenched with 1 N HCl (100 mL), extracted with ethyl acetate (3×), dried over Na₂SO₄, evaporated under reduced pressure, the residue was purified by flash chromatography on silica gel (100 g column) eluted with 0 to 20% MeOH/DCM to afford 4.84 g (93.7%) of (E)-2-((benzyloxy)imino)-2-(2-chloro-3,4-dimethoxyphenyl)acetic acid as a solid. ESI-MS m/z 249.8, 351.8 (M+H)⁺, 371.7, 373.7 (M+Na)⁺.

### Step 5. Synthesis of tert-butyl 3-((R)-2-((E)-2-((benzyloxy)imino)-2-(2-chloro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

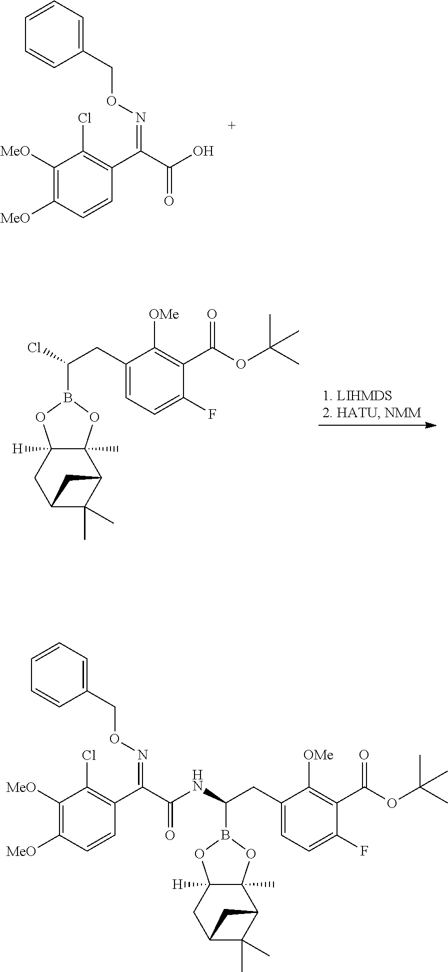 [SMILES: COc1ccc(/C(=N\OCc2ccccc2)C(=O)O)c(Cl)c1OC ; COc1c(C[C@@H](Cl)B2O[C@@H]3C[C@@H]4C[C@@H](C4(C)C)[C@]3(C)O2)ccc(F)c1C(=O)OC(C)(C)C ; COc1ccc(/C(=N\OCc2ccccc2)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

To a solution of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (3.23 g, 6.92 mmol) in THF (24 mL) at −78° C. under argon was added LiHMDS (1.0 M in THF, 7.5 mL, 7.5 mmol) dropwise. The resulting mixture was allowed to slowly warm to room temperature overnight.

A mixture of (E)-2-((benzyloxy)imino)-2-(2-chloro-3,4-dimethoxyphenyl)acetic acid (3.22 g, 9.21 mmol), HATU (8.50 g, 22.3 mmol), NMM (4.0 mL, 36.4 mmol) in DMA (35 mL) was stirred at room temperature under argon for 1 h and then added to the reaction mixture above via cannula.

The resulting reaction mixture was stirred at room temperature for 33 h and then quenched with water, extracted with ethyl acetate (3×). The combined organic phase was dried over Na₂SO₄. After the solvent was evaporated under reduced pressure, the residue was purified by flash chromatography on silica gel (100 g column) eluted with 0 to 40% ethyl acetate/hexanes to afford 4.30 g (79.8%) of tert-butyl 3-((R)-2-((E)-2-((benzyloxy)imino)-2-(2-chloro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate as a solid. ESI-MS m/z 722.5 (MH⁺-56), 778.5 (M+H)⁺, 800.5 (M+Na)⁺.

### Step 6. Synthesis of tert-butyl 3-((2R)-2-(2-amino-2-(3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

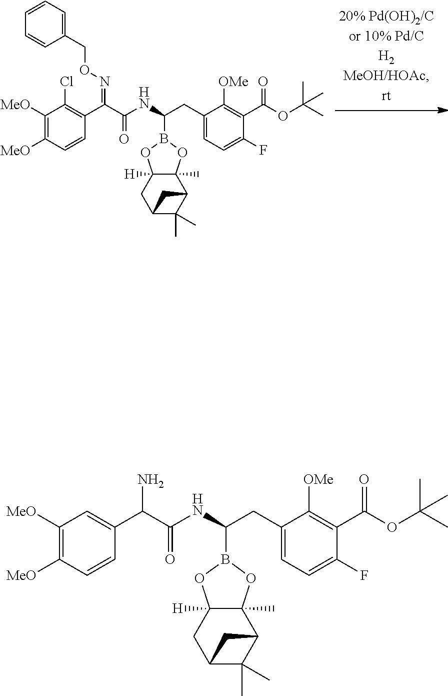 [SMILES: COc1ccc(/C(=N\OCc2ccccc2)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; COc1ccc(C(N)C(=O)N[C@@H](Cc2ccc(F)c(C(=O)OC(C)(C)C)c2OC)B2O[C@@H]3C[C@@H]4C[C@@H](C4(C)C)[C@]3(C)O2)cc1OC]

A mixture of tert-butyl 3-((R)-2-((E)-2-((benzyloxy)imino)-2-(2-chloro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (0.130 g, 0.167 mmol) and 20% Pd(OH)₂/C (0.060 g) in HOAc (3 mL) and MeOH (12 mL) was stirred at room temperature under hydrogen balloon for 16 h. The mixture was then filtered through HPLC filter. The filtrate was evaporated under reduced pressure and the crude product was used in the next step directly. ESI-MS m/z 640.7 (M+H)⁺, 662.6 (M+Na)⁺.

### Step 7. Synthesis of tert-butyl 3-((2R)-2-(2-(3,4-dimethoxyphenyl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

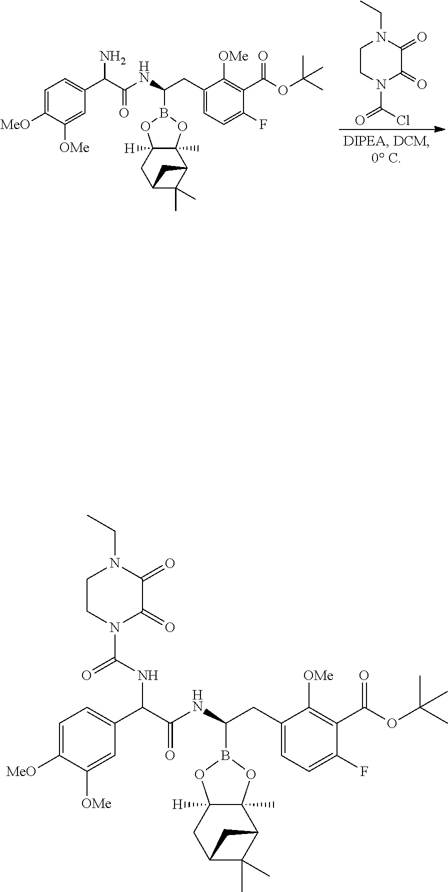

To a mixture of tert-butyl 3-((2R)-2-(2-amino-2-(3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate, obtained as described above, and DIPEA (0.5 mL) in DCM (10 mL) at 0° C. was added 4-ethyl-2,3-dioxopiperazine-1-carbonyl chloride (0.116 g). The reaction mixture was stirred at 0° C. for 2 h and then evaporated under reduced pressure. The crude product was used in the next step directly. ESI-MS m/z 752.5 (MH⁺-56), 808.6 (M+H)⁺, 830.5 (M+Na)⁺.

### Step 8. Synthesis of (R)-3-((S)-2-(3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid and (R)-3-((R)-2-(3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

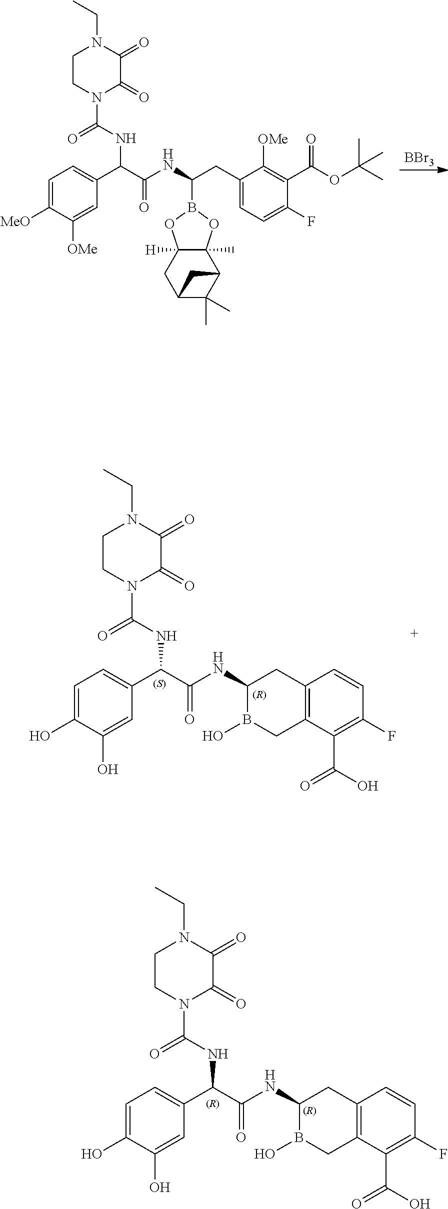

The full deprotection of tert-butyl 3-((2R)-2-(2-(3,4-dimethoxyphenyl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate, obtained as described above, was carried out as described in General Method A with BBr₃ to afford 14.18 mg of (R)-3-((S)-2-(3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid, ESI-MS m/z 558.6 (M+H)⁺, 580.6 (M+Na)*, and 25.32 mg of (R)-3-((R)-2-(3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid. ESI-MS m/z 540.6 (MH⁺-18), 558.6 (M+H)⁺, 580.5 (M+Na)⁺.

### Example 212: (3R)-3-(2-(4-(azetidin-3-yl)-2,3-dioxopiperazine-1-carboxamido)-2-(2,6-difluoro-4-hydroxyphenyl)acetamido)-2-hydroxy-3,4-dihydro-2H-benzo[e][1,2]oxaborinine-8-carboxylic acid

### Step 1: Synthesis of tert-butyl 3-((2-aminoethyl)amino)azetidine-1-carboxylate

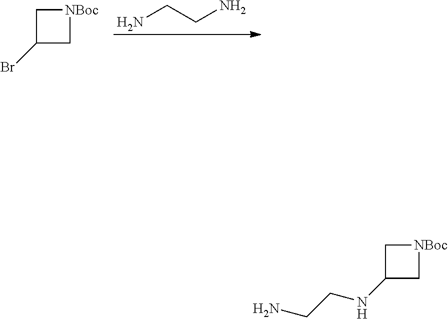

To ethane-1,2-diamine 99 mL (148 mmol, 7 eq) was added slowly over 20 min tert-butyl 3-bromoazetidine-1-carboxylate 5 g (212 mmol) and stirred at 80° C. for 18 h. The reaction was cooled at RT, extracted with diethyl ether 2×, and concentrated in vacuo to give the title compound, 4.47 g, (98%). ESI-MS m/z 216 (MH)⁺.

### Step 2: Synthesis of tert-butyl 3-(4-(chlorocarbonyl)-2,3-dioxopiperazin-1-yl)azetidine-1-carboxylate

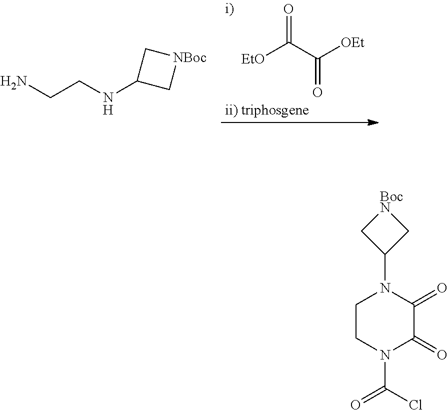 [SMILES: CC(C)(C)OC(=O)N1CC(NCCN)C1 ; CCOC(=O)C(=O)OCC ; CC(C)(C)OC(=O)N1CC(N2CCN(C(=O)Cl)C(=O)C2=O)C1]

By following the same procedures as described for the synthesis of tert-butyl (2-(4-(chlorocarbonyl)-2,3-dioxopiperazin-1-yl)ethyl)carbamate in Step 1b and 1c of Example 26, the title compound was prepared from tert-butyl 3-((2-aminoethyl)amino)azetidine-1-carboxylate.

### Step 3: Synthesis of (3R)-3-(2-(4-(azetidin-3-yl)-2,3-dioxopiperazine-1-carboxamido)-2-(2,6-difluoro-4-hydroxyphenyl)acetamido)-2-hydroxy-3,4-dihydro-2H-benzo[e][1,2]oxaborinine-8-carboxylic acid

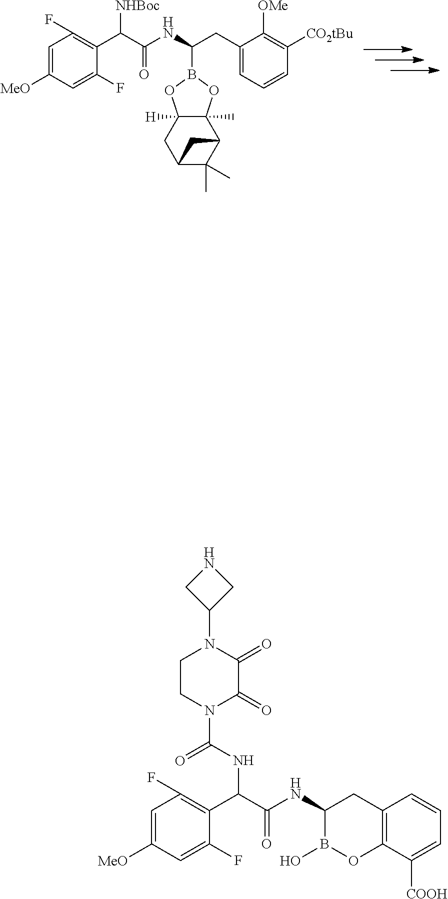 [SMILES: COc1cc(F)c(C(NC(=O)OC(C)(C)C)C(=O)N[C@@H](Cc2cccc(C(=O)OC(C)(C)C)c2OC)B2O[C@@H]3C[C@@H]4C[C@@H](C4(C)C)[C@]3(C)O2)c(F)c1 ; COc1cc(F)c(C(NC(=O)N2CCN(C3CNC3)C(=O)C2=O)C(=O)N[C@H]2Cc3cccc(C(=O)O)c3OB2O)c(F)c1]

By following the general deprotection and purification method, the above compound was treated with excess BBr₃ and stirred at RT for 18 h to yield the title compound after reversed phase HPLC purification. ESI-MS m/z 588 (MH)⁺.

### Example 213: (3R)-3-(2-(2,6-difluoro-4-(3-methylureido)phenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 188, utilizing methylcarbamic chloride in place of acetyl chloride. ESI-MS m/z 617 (MH)⁺.

### Example 215: (R)-3-((R)-2-(2,6-difluoro-4-hydroxyphenyl)-2-(2,3-dioxo-4-(2,2,2-trifluoroethyl)piperazine-1-carboxamido)acetamido)-2-hydroxy-3,4-dihydro-2H-benzo[e][1,2]oxaborinine-8-carboxylic acid

### Step 1. Synthesis of 2,3-dioxo-4-(2,2,2-trifluoroethyl)piperazine-1-carbonyl chloride

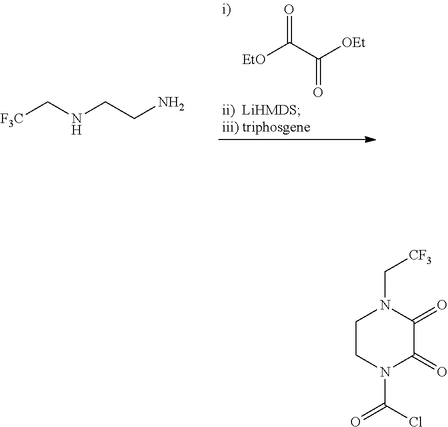 [SMILES: CCOC(=O)C(=O)OCC ; NCCNCC(F)(F)F ; O=C1C(=O)N(C(=O)Cl)CCN1CC(F)(F)F]

### Step 1a

To the EtOH (300 mL) solution of N¹-(2,2,2-trifluoroethyl)ethane-1,2-diamine (5 g, 35.2 mmol) was added diethyl oxalate (6 mL, 44.2 mmol). The reaction mixture was stirred at reflux for 2 days, then concentrated in vacuo.

### Step 1b

To the THF (200 mL) solution of the above crude product was added at −30° C. LiHMDS (1.0 M, 45 mL, 45 mmol). The reaction mixture was warmed to RT, stirred for 5 h, concentrated in vacuo, acidified with 1 N HCl to pH=3-4, then freeze-dried. The solid was treated with a mixed solvent of MeOH-DCM (1:3) for several times, the extracts were combined, concentrated in vaccuo, the residue was purified by flash chromatography on silica gel (DCM-MeOH, 20:1-4:1) to afford the product, 2 g. ESI-MS m/z 197 (M+H)⁺.

### Step 1c

To the above product (2 g, 10.2 mmol) in THF (28 mL) and DCM (14 mL) at −15° C. was added chlorotrimethylsilane (1.44 mL, 11.3 mmol), followed by triethylamine (1.7 mL, 12.1 mmol). The reaction mixture was stirred between −15° C.-0° C. for 1 h, then triphosgene (1.23 g, 4.1 mmol) in THF (5 mL) was added dropwise to the reaction mixture. After addition was complete, the reaction mixture was warmed up to RT over 30 min, stirred for an additional 2 h, and the solid was filtered off and washed with THF. The filtrate was concentrated in vacuo to give the crude title compound which was used directly for the next step without further purification.

### Step 2. Synthesis of (R)-3-((R)-2-(2,6-difluoro-4-hydroxyphenyl)-2-(2,3-dioxo-4-(2,2,2-trifluoroethyl)piperazine-1-carboxamido)acetamido)-2-hydroxy-3,4-dihydro-2H-benzo[e][1,2]oxaborinine-8-carboxylic acid

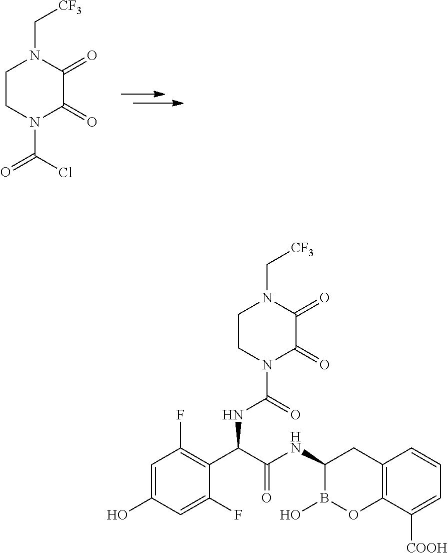 [SMILES: O=C1C(=O)N(C(=O)Cl)CCN1CC(F)(F)F ; O=C(O)c1cccc2c1OB(O)[C@@H](NC(=O)[C@H](NC(=O)N1CCN(CC(F)(F)F)C(=O)C1=O)c1c(F)cc(O)cc1F)C2]

In a similar manner to the synthesis of Example 105, utilizing 2,3-dioxo-4-(2,2,2-trifluoroethyl)piperazine-1-carbonyl chloride in place of 4-ethyl-2,3-dioxopiperazine-1-carbonyl chloride, the title compound was isolated as the second eluting peak after reversed phase HPLC purification. ESI-MS m/z 615 (M+1)⁺.

### Example 216: (R)-3-((S)-2-(2,3-dioxo-4-(2,2,2-trifluoroethyl)piperazine-1-carboxamido)-2-(3-(sulfamoylamino)phenyl)acetamido)-2-hydroxy-3,4-dihydro-2H-benzo[e][1,2]oxaborinine-8-carboxylic acid

### Example 217: (R)-3-((R)-2-(2,3-dioxo-4-(2,2,2-trifluoroethyl)piperazine-1-carboxamido)-2-(3-(sulfamoylamino)phenyl)acetamido)-2-hydroxy-3,4-dihydro-2H-benzo[e][1,2]oxaborinine-8-carboxylic acid

In a similar manner to the synthesis of Example 114, 115, utilizing 2,3-dioxo-4-(2,2,2-trifluoroethyl)piperazine-1-carbonyl chloride in place of tert-butyl (2-(4-(chlorocarbonyl)-2,3-dioxopiperazin-1-yl)ethyl)carbamate, Example 216 was isolated as the first eluting peak, and Example 217 was isolated as the second eluting peak prepared after reversed phase HPLC purification. ESI-MS m/z 657 (M+1)⁺.

### Example 218: (R)-3-((S)-2-(2,3-dioxo-4-(2,2,2-trifluoroethyl)piperazine-1-carboxamido)-2-(2-fluoro-5-(sulfamoylamino)phenyl)acetamido)-2-hydroxy-3,4-dihydro-2H-benzo[e][1,2]oxaborinine-8-carboxylic acid

### Example 219: (R)-3-((R)-2-(2,3-dioxo-4-(2,2,2-trifluoroethyl)piperazine-1-carboxamido)-2-(2-fluoro-5-(sulfamoylamino)phenyl)acetamido)-2-hydroxy-3,4-dihydro-2H-benzo[e][1,2]oxaborinine-8-carboxylic acid

In a similar manner to the synthesis of Example 197, utilizing 2,3-dioxo-4-(2,2,2-trifluoroethyl)piperazine-1-carbonyl chloride in place of 4-ethyl-2,3-dioxopiperazine-1-carbonyl chloride, the title compound, Example 218 was isolated as the first eluting peak, and Example 219 was isolated as the second eluting peak prepared after reversed phase HPLC purification. ESI-MS m/z 675 (M+1)⁺.

### Example 220: (3R)-3-(2-(4-carboxy-3-nitrophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 17, utilizing 2-(4-(tert-butoxycarbonyl)-3-nitrophenyl)-2-((tert-butoxycarbonyl)amino)acetic acid in step 2, utilizing tert-butyl 3-((2R)-2-(2-(4-(tert-butoxycarbonyl)-3-nitrophenyl)-2-((tert-butoxycarbonyl)amino)acetamido)-2-((3aS,4S,6S,7aR)-3a,5,5-trimethylhexahydro-4,6-methanobenzo[d][1,3,2]dioxaborol-2-yl)ethyl)-2-methoxybenzoate in step 3, utilizing tert-butyl 3-((2R)-2-(2-amino-2-(4-(tert-butoxycarbonyl)-3-nitrophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate in step 4, utilizing tert-butyl 3-((2R)-2-(2-(4-(tert-butoxycarbonyl)-3-nitrophenyl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate in step 6, the title compound was prepared. ESI-MS m/z 597.6 (MH)⁺.

### Example 221: (R)-3-((S)-2-(4-carboxy-3-chloro-2-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 157, utilizing 2-((tert-butoxycarbonyl)amino)-2-(2,5-dichloro-4-methoxyphenyl)acetic acid in place of 2-(4-(tert-butoxycarbonyl)-2,6-difluorophenyl)-2-((tert-butoxycarbonyl)amino)acetic acid, isolated as the first eluting peak. ESI-MS m/z 605 (MH)⁺.

### Example 222: (R)-3-((R)-2-(4-carboxy-3-chloro-2-fluorophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 221, the title compound was prepared after reversed phase HPLC purification, isolated as the second eluting peak. ESI-MS m/z 605 (MH)⁺.

### Example 223: (R)-3-((R)-2-(4-(2-aminoethyl)-2,3-dioxopiperazine-1-carboxamido)-2-(2,6-difluorophenyl)acetamido)-2-hydroxy-3,4-dihydro-2H-benzo[e][1,2]oxaborinine-8-carboxylic acid

The title compound was prepared in a similar manner to the synthesis of Example 204, the title compound was prepared after reversed phase HPLC purification, isolated as the first eluting peak. ESI-MS m/z 560 (MH)⁺.

### Example 224: (R)-3-((R)-2-(4-(2-aminoethyl)-2,3-dioxopiperazine-1-carboxamido)-2-(2,6-dichlorophenyl)acetamido)-2-hydroxy-3,4-dihydro-2H-benzo[e][1,2]oxaborinine-8-carboxylic acid

The title compound was prepared in a similar manner to the synthesis of Example 17, utilizing 2-((tert-butoxycarbonyl)amino)-2-(2,6-dichlorophenyl)acetic acid in place of 2-((tert-butoxycarbonyl)amino)-2-(4-methoxy-3-nitrophenyl)acetic acid, isolated as the first eluting peak. ESI-MS m/z 592 (MH)⁺.

### Example 225: (R)-3-((S)-2-(4-(2-aminoethyl)-2,3-dioxopiperazine-1-carboxamido)-2-(2,6-dichlorophenyl)acetamido)-2-hydroxy-3,4-dihydro-2H-benzo[e][1,2]oxaborinine-8-carboxylic acid

The title compound was prepared in a similar manner to the synthesis of Example 224, the title compound was prepared after reversed phase HPLC purification, isolated as the second eluting peak. ESI-MS m/z 592 (MH)⁺.

### Example 226: (R)-3-((R)-2-(4-(2-aminoethyl)-2,3-dioxopiperazine-1-carboxamido)-2-(2-chloro-5,6-difluoro-3,4-dihydroxyphenyl)acetamido)-7-fluoro-2-hydroxy-3,4-dihydro-2H-benzo[e][1,2]oxaborinine-8-carboxylic acid

### Step 1. Synthesis of 2-((tert-butoxycarbonyl)amino)-2-(2-chloro-5,6-difluoro-3,4-dimethoxyphenyl)acetic acid

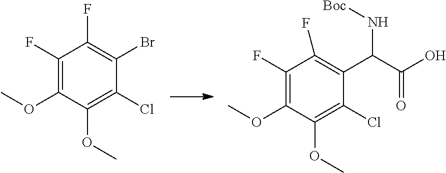 [SMILES: COc1c(F)c(F)c(Br)c(Cl)c1OC ; COc1c(F)c(F)c(C(NC(=O)OC(C)(C)C)C(=O)O)c(Cl)c1OC]

The title compound was prepared in a similar manner to the synthesis steps 1˜5 of Example 157, utilizing 1-bromo-2-chloro-5,6-difluoro-3,4-dimethoxybenzene in place of tert-butyl 4-bromo-3,5-difluorobenzoate. ESI-MS m/z 382.1 (M+H)⁺.

### Step 2: Synthesis of tert-butyl 3-((2R)-2-(2-((tert-butoxycarbonyl)amino)-2-(2-chloro-5,6-difluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

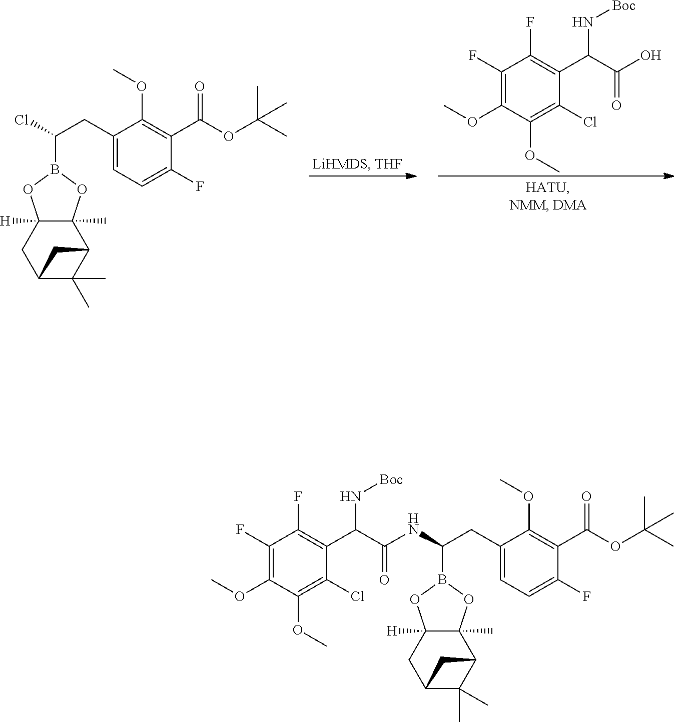 [SMILES: COc1c(C[C@@H](Cl)B2O[C@@H]3C[C@@H]4C[C@@H](C4(C)C)[C@]3(C)O2)ccc(F)c1C(=O)OC(C)(C)C ; COc1c(F)c(F)c(C(NC(=O)OC(C)(C)C)C(=O)O)c(Cl)c1OC ; COc1c(F)c(F)c(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

By following the General Procedure C, the chloride (prepared as previous reported, WO 2014/089365) was treated with LiHMDS, and then coupled 2-((tert-butoxycarbonyl)amino)-2-(2-chloro-5,6-difluoro-3,4-dimethoxyphenyl)acetic acid in the presence of HATU and NMM, yielding the title compound. ESI-MS m/z 811.3 (M+H)⁺.

### Step 3: Synthesis of (R)-3-((R)-2-(4-(2-aminoethyl)-2,3-dioxopiperazine-1-carboxamido)-2-(2-chloro-5,6-difluoro-3,4-dihydroxyphenyl)acetamido)-7-fluoro-2-hydroxy-3,4-dihydro-2H-benzo[e][1,2]oxaborinine-8-carboxylic acid

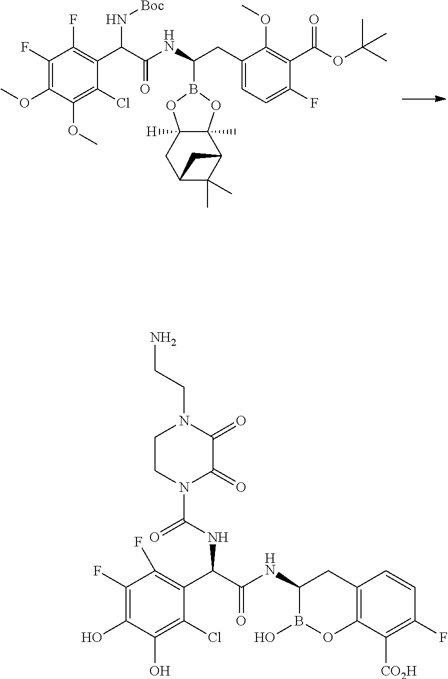 [SMILES: COc1c(F)c(F)c(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; NCCN1CCN(C(=O)N[C@@H](C(=O)N[C@H]2Cc3ccc(F)c(C(=O)O)c3OB2O)c2c(F)c(F)c(O)c(O)c2Cl)C(=O)C1=O]

The title compound was prepared in a similar manner to the synthesis of Example 1, utilizing 4-ethyl-2,3-dioxopiperazine-1-carbonyl chloride in place of tert-butyl (3-(4-(chlorocarbonyl)-2,3-dioxopiperazin-1-yl)propyl)carbamate. ESI-MS m/z 644.1 (M+H)⁺.

### Example 227: (R)-3-((R)-2-(4-(3-aminopropyl)-2,3-dioxopiperazine-1-carboxamido)-2-(2-chloro-5,6-difluoro-3,4-dihydroxyphenyl)acetamido)-7-fluoro-2-hydroxy-3,4-dihydro-2H-benzo[e][1,2]oxaborinine-8-carboxylic acid

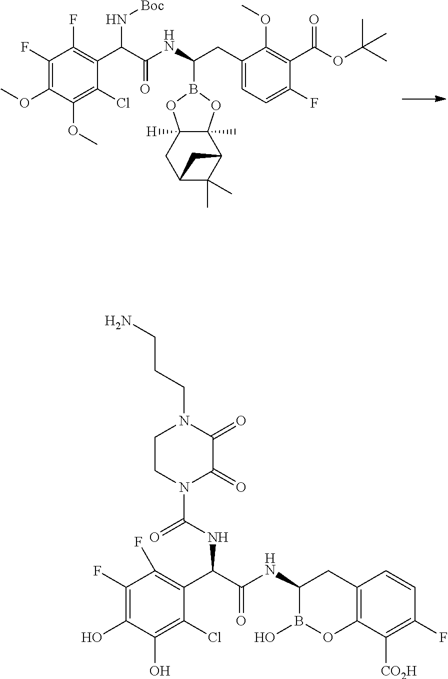 [SMILES: COc1c(F)c(F)c(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; NCCCN1CCN(C(=O)N[C@@H](C(=O)N[C@H]2Cc3ccc(F)c(C(=O)O)c3OB2O)c2c(F)c(F)c(O)c(O)c2Cl)C(=O)C1=O]

The title compound was prepared in a similar manner to the synthesis of Example 1, utilizing tert-butyl (3-(4-(chlorocarbonyl)-2,3-dioxopiperazin-1-yl)propyl)carbamate in place of tert-butyl (3-(4-(chlorocarbonyl)-2,3-dioxopiperazin-1-yl)propyl)carbamate. ESI-MS m/z 658.1 (M+H)⁺.

### Example 228: (R)-3-((R)-2-(2-chloro-5,6-difluoro-3,4-dihydroxyphenyl)-2-(4-ethyl-2,3-dioxopiperazine-1-carboxamido)acetamido)-7-fluoro-2-hydroxy-3,4-dihydro-2H-benzo[e][1,2]oxaborinine-8-carboxylic acid

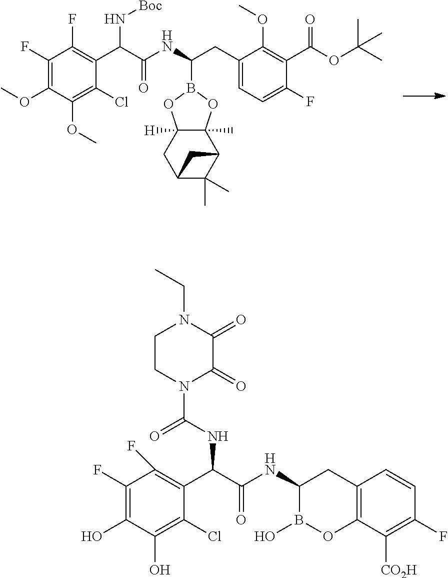 [SMILES: COc1c(F)c(F)c(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; CCN1CCN(C(=O)N[C@@H](C(=O)N[C@H]2Cc3ccc(F)c(C(=O)O)c3OB2O)c2c(F)c(F)c(O)c(O)c2Cl)C(=O)C1=O]

The title compound was prepared in a similar manner to the synthesis of Example 1, utilizing 4-ethyl-2,3-dioxopiperazine-1-carbonyl chloride in place of tert-butyl (3-(4-(chlorocarbonyl)-2,3-dioxopiperazin-1-yl)propyl)carbamate. ESI-MS m/z 629.1 (M+H)⁺.

### Example 229: (R)-3-((R)-2-(2-chloro-5,6-difluoro-3,4-dihydroxyphenyl)-2-(3-(methylsulfonyl)-2-oxoimidazolidine-1-carboxamido)acetamido)-7-fluoro-2-hydroxy-3,4-dihydro-2H-benzo[e][1,2]oxaborinine-8-carboxylic acid

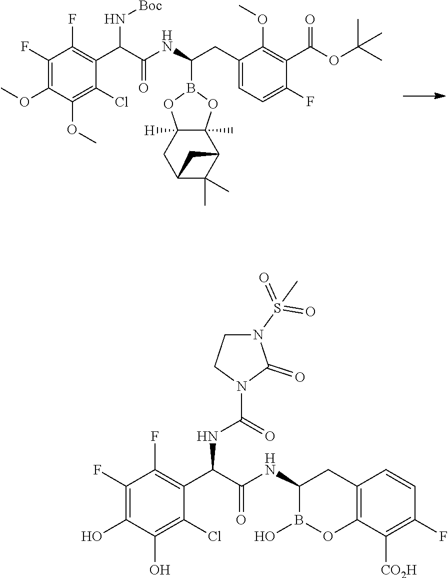 [SMILES: COc1c(F)c(F)c(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; CS(=O)(=O)N1CCN(C(=O)N[C@@H](C(=O)N[C@H]2Cc3ccc(F)c(C(=O)O)c3OB2O)c2c(F)c(F)c(O)c(O)c2Cl)C1=O]

The title compound was prepared in a similar manner to the synthesis of Example 1, utilizing 3-(methylsulfonyl)-2-oxoimidazolidine-1-carbonyl chloride in place of tert-butyl (3-(4-(chlorocarbonyl)-2,3-dioxopiperazin-1-yl)propyl)carbamate. ESI-MS m/z 551.1 (M+H)⁺.

### Example 230: (R)-3-((R)-2-(3-(2-aminoethyl)-2-oxohexahydropyrimidine-1-carboxamido)-2-(2,6-difluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2,6-difluoro-4-methoxyphenyl)acetic acid

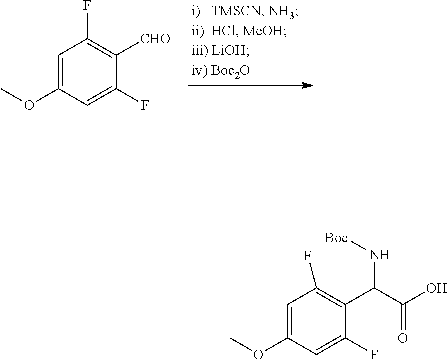 [SMILES: COc1cc(F)c(C=O)c(F)c1 ; COc1cc(F)c(C(NC(=O)OC(C)(C)C)C(=O)O)c(F)c1]

The title compound was prepared in a similar manner to the synthesis step 1 of Example 17, utilizing 2,6-difluoro-4-methoxybenzaldehyde in place of 4-methoxy-3-nitrobenzaldehyde. ESI-MS m/z 318.1 (M+H)⁺.

### Step 2: Synthesis of tert-butyl 3-((2R)-2-(2-((tert-butoxycarbonyl)amino)-2-(2,6-difluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

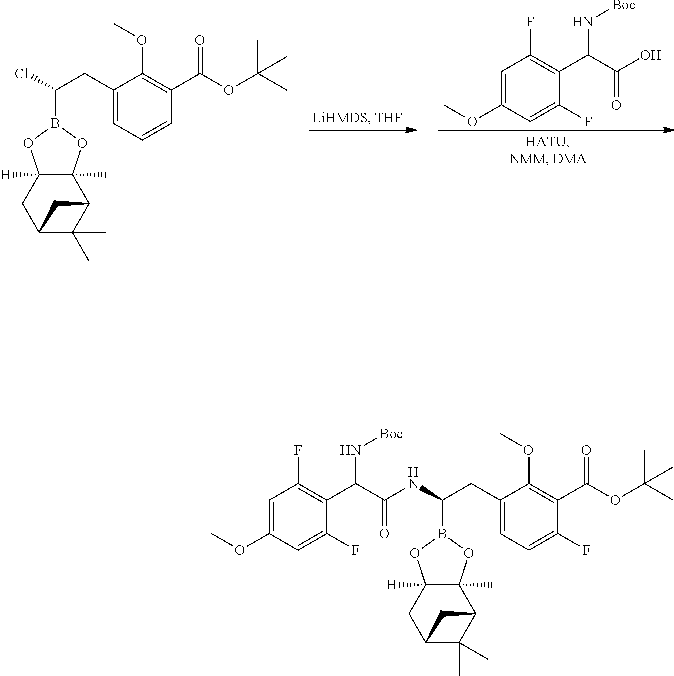 [SMILES: COc1c(C[C@@H](Cl)B2O[C@@H]3C[C@@H]4C[C@@H](C4(C)C)[C@]3(C)O2)cccc1C(=O)OC(C)(C)C ; COc1cc(F)c(C(NC(=O)OC(C)(C)C)C(=O)O)c(F)c1 ; COc1cc(F)c(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(F)c1]

By following the General Procedure C, the chloride (prepared as previous reported, WO 2014/089365) was treated with LiHMDS, and then coupled 2-((tert-butoxycarbonyl)amino)-2-(2,6-difluoro-4-methoxyphenyl)acetic acid in the presence of HATU and NMM, yielding the title compound. ESI-MS m/z 729.3 (M+H)⁺.

### Step 3: Synthesis of (R)-3-((R)-2-(3-(2-aminoethyl)-2-oxohexahydropyrimidine-1-carboxamido)-2-(2,6-difluoro-4-hydroxyphenyl)acetamido)-2-hydroxy-3,4-dihydro-2H-benzo[e][1,2]oxaborinine-8-carboxylic acid

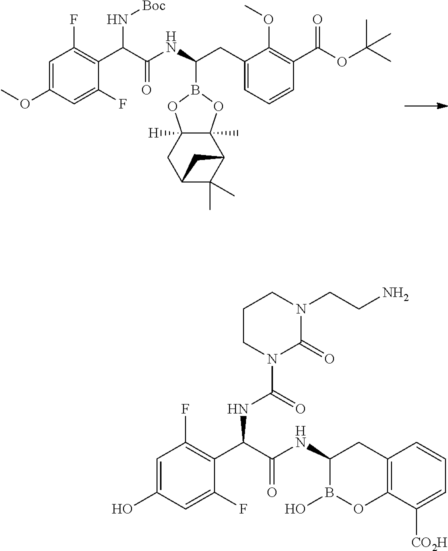 [SMILES: COc1cc(F)c(C(NC(=O)OC(C)(C)C)C(=O)N[C@@H](Cc2cccc(C(=O)OC(C)(C)C)c2OC)B2O[C@@H]3C[C@@H]4C[C@@H](C4(C)C)[C@]3(C)O2)c(F)c1 ; NCCN1CCCN(C(=O)N[C@@H](C(=O)N[C@H]2Cc3cccc(C(=O)O)c3OB2O)c2c(F)cc(O)cc2F)C1=O]

The title compound was prepared in a similar manner to the synthesis of Example 1, utilizing tert-butyl (2-(3-(chlorocarbonyl)-2-oxotetrahydropyrimidin-1(2H)-yl)ethyl)carbamate in place of tert-butyl (3-(4-(chlorocarbonyl)-2,3-dioxopiperazin-1-yl)propyl)carbamate, isolated as the first eluting peak. ESI-MS m/z 561.7 (M+H)⁺.

### Example 231: (R)-3-((S)-2-(3-(2-aminoethyl)-2-oxohexahydropyrimidine-1-carboxamido)-2-(2,6-difluoro-4-hydroxyphenyl)acetamido)-2-hydroxy-3,4-dihydro-2H-benzo[e][1,2]oxaborinine-8-carboxylic acid

The title compound was prepared in a same manner to the synthesis of Example 230, via the purification of reverse phase HPLC, isolated as the second eluting peak. ESI-MS m/z 561.6 (M+H)⁺.

### Example 232: (3R)-3-(2-(2,6-difluoro-4-hydroxyphenyl)-2-((S)-4-ethyl-6-methyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 17, utilizing 2-((tert-butoxycarbonyl)amino)-2-(2,6-difluoro-4-methoxyphenyl)acetic acid in place of 2-((tert-butoxycarbonyl)amino)-2-(4-methoxy-3-nitrophenyl)acetic acid. ESI-MS m/z 575 (MH)⁺.

### Example 233: (3R)-3-(2-(2,6-difluoro-4-hydroxyphenyl)-2-((S)-4-(2-fluoroethyl)-6-methyl-2,3-dioxopiperazine-1-carboxamido)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2,6-difluoro-4-methoxyphenyl)acetic acid

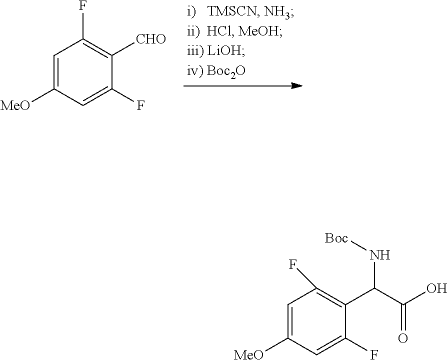 [SMILES: COc1cc(F)c(C=O)c(F)c1 ; COc1cc(F)c(C(NC(=O)OC(C)(C)C)C(=O)O)c(F)c1]

In a same manner to the synthesis of Step 1 of Example 17, the title compound was prepared from 2,6-difluoro-4-methoxybenzaldehyde.

### Step 2. Synthesis of tert-butyl 3-((2R)-2-(2-((tert-butoxycarbonyl)amino)-2-(2,6-difluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

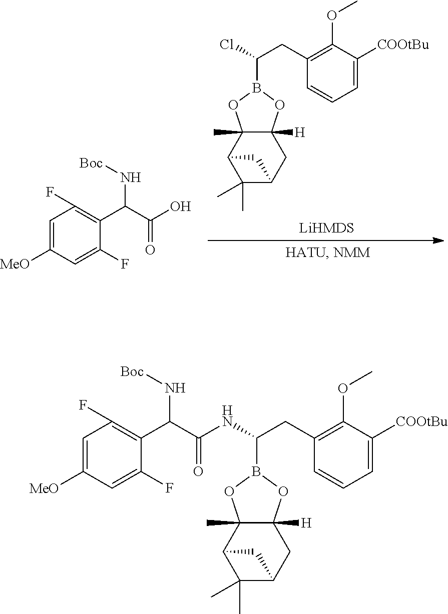

By following the General coupling method C, the title compound was prepared from the above acid and the chloride intermediate (prepared as previous reported, WO 2014/089365). ESI-MS m/z 673 (M+H-56)⁺.

### Step 3. Synthesis of (S)-4-(2-fluoroethyl)-6-methyl-2,3-dioxopiperazine-1-carbonyl chloride

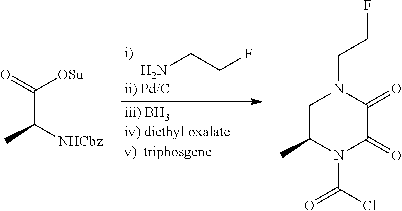

By following the same procedure as described for the Synthesis of tert-butyl (S)-(2-(4-(chloroarbonyl)-5-methyl-2,3-dioxopiperazin-1-yl)ethyl)carbamate in Step 1 of Example 234, the title compound was prepared from 2-fluoroethan-1-amine.

### Step 4. Synthesis of (3R)-3-(2-(2,6-difluoro-4-hydroxyphenyl)-2-((S)-4-(2-fluoroethyl)-6-methyl-2,3-dioxopiperazine-1-carboxamido)acetamido)-2-hydroxy-3,4-dihydro-2H-benzo[e][1,2]oxaborinine-8-carboxylic acid

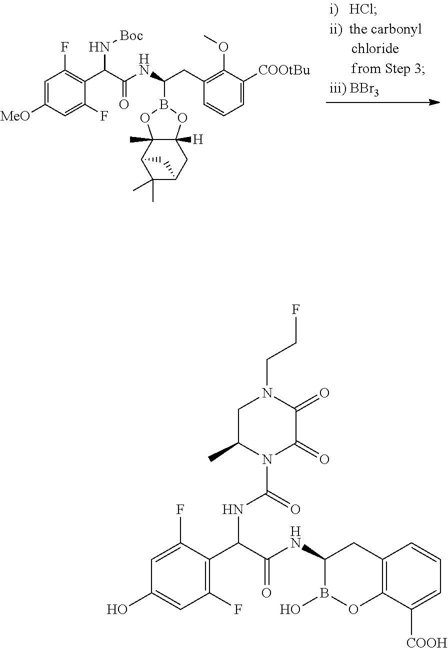

In a similar manner to the synthesis of Example 26, the title compound was prepared after reversed phase HPLC purification. ESI-MS m/z 593 (M+H)⁺.

### Example 234: (3R)-3-(2-((S)-4-(2-aminoethyl)-6-methyl-2,3-dioxopiperazine-1-carboxamido)-2-(2,6-difluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2,6-difluoro-4-methoxyphenyl)acetic acid

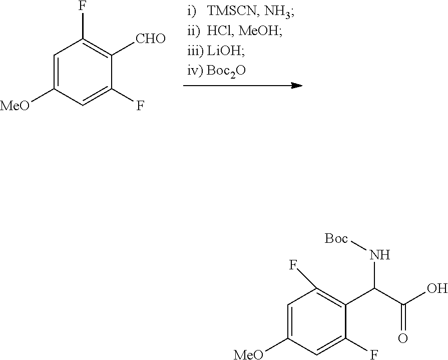 [SMILES: COc1cc(F)c(C=O)c(F)c1 ; COc1cc(F)c(C(NC(=O)OC(C)(C)C)C(=O)O)c(F)c1]

In a same manner to the synthesis of Step 1 of Example 17, the title compound was prepared from 2,6-difluoro-4-methoxybenzaldehyde.

### Step 2. Synthesis of tert-butyl 3-((2R)-2-(2-((tert-butoxycarbonyl)amino)-2-(2,6-difluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

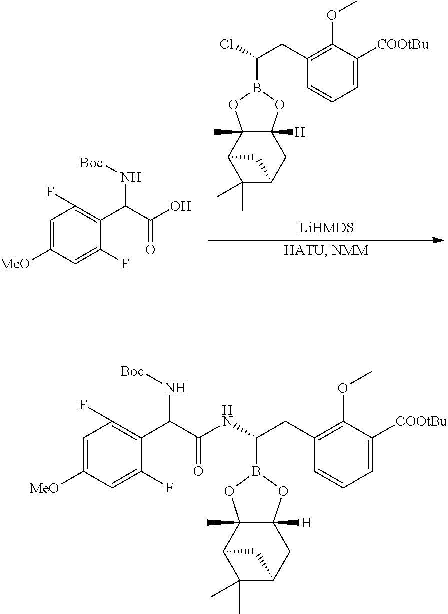

By following the General coupling method C, the title compound was prepared from the above acid and the chloride intermediate (prepared as previous reported, WO 2014/089365). ESI-MS m/z 673 (M+H-56)⁺.

### Step 3. Synthesis of tert-butyl (S)-(2-(4-(chloroarbonyl)-5-methyl-2,3-dioxopiperazin-1-yl)ethyl)carbamate

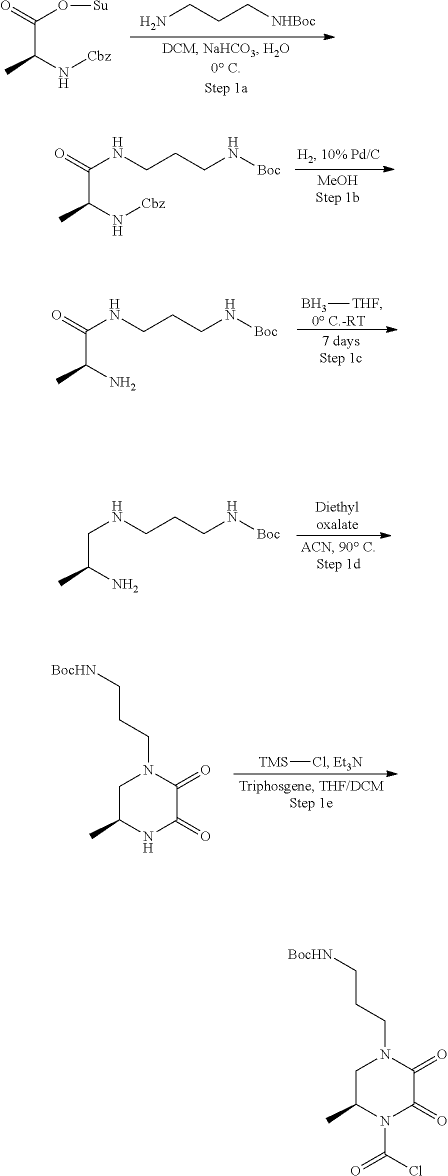

### Step 1a

To a mixture of Z-Ala-OSu (16.18 g, 50.5 mmol) in DCM (300 mL) was added dropwise tert butyl N-(2-aminoethyl) carbamate (8.83 g, 55.1 mmol) at 0° C. The mixture was allowed to slowly warm to room temperature overnight. After 23 h, the reaction mixture was quenched with saturated NaHCO₃, extracted with DCM (3×100 mL), dried over Na₂SO₄. After the solvent was evaporated under reduced pressure, the residue was purified by flash chromatography on silica gel (120 g column) eluted with 0 to 10% MeOH/DCM to afford 13.55 g of benzyl (S)-(1-((2-((tert-butoxycarbonyl)amino)ethyl)amino)-1-oxopropan-2-yl)carbamate. ESI-MS m/z 388.2 (M+Na)⁺, 310.1 (M+H-56)+, 266.2 (M+H-Boc)⁺.

### Step 1b

A mixture of benzyl (S)-(1-((2-((tert-butoxycarbonyl)amino)ethyl)amino)-1-oxopropan-2-yl)carbamate (13.55 g, 37.08 mmol) and 10% Pd/C (wet support, 2.08 g) in MeOH (200 mL) was stirred under hydrogen balloon for 2 h. The mixture was then filtered, washed with MeOH. The filtrate was evaporated under reduced pressure to afford tert-butyl (S)-(2-(2 aminopropanamido) ethyl)carbamate (8.93 g), which was used in the next step directly. ESI-MS m/z 232.2 (M+H)⁺.

### Step 1c

To a solution of tert-butyl (S)-(2-(2-aminopropanamido)ethyl)carbamate (4.36 g, 18.8 mmol) in THF (40 mL) was added dropwise a solution of Borane tetrahydrofuran complex solution (1.0 M in THF, 100 mL, 100 mmol) at 0° C. The mixture was allowed to slowly warm to room temperature overnight. After 7 d, the reaction mixture was carefully quenched with MeOH at 0° C. The mixture was then evaporated under reduced pressure to afford tert-butyl (S)-(2-((2-aminopropyl)amino)ethyl)carbamate (3.48 g), which was used in the next step directly. ESI-MS m/z 218.2 (M+H)⁺.

### Step 1d

A mixture of tert-butyl (S)-(2-((2-aminopropyl)amino)ethyl)carbamate (3.48 g), obtained as described above, and diethyl oxalate (2.339 g, 16 mmol) in CH₃CN (50 mL) was heated at 90° C. for 18 h. After the solvent was evaporated under reduced pressure, the residue was purified by flash chromatography on silica gel (100 g column) eluted with 0 to 15% MeOH/DCM to afford 2 g of tert-butyl (S)-(2-(5-methyl-2,3-dioxopiperazin-1-yl)ethyl)carbamate. ESI-MS m/z 565.3 (2M+Na)⁺, 443.2 (2M+H-Boc)⁺, 216.1 (M+H-56)⁺.

### Step 1e

To a solution of tert-butyl (S)-(2-(5-methyl-2,3-dioxopiperazin-1-yl)ethyl)carbamate (0.51 g, 1.88 mmol) in THF (6 mL) and DCM (6 mL) were added dropwise TMSCl (0.3 mL, 2.36 mmol) and TEA (0.33 mL, 2.37 mmol) at −65° C. under argon. After 3 h, the reaction mixture was then stirred at 0° C. for an additional 2 h. A solution of triphosgene (0.30 g, 1.01 mmol) in THF (10 mL) was added at 0° C. The mixture was allowed to slowly warm to room temperature overnight and then filtered, washed with THF. The filtrate was concentrated in vacuo to give the crude title compound which was used directly for the next step without further purification.

### Step 4. Synthesis of (3R)-3-(2-((S)-4-(2-aminoethyl)-6-methyl-2,3-dioxopiperazine-1-carboxamido)-2-(2,6-difluoro-4-hydroxyphenyl)acetamido)-2-hydroxy-3,4-dihydro-2H-benzo[e][1,2]oxaborinine-8-carboxylic acid

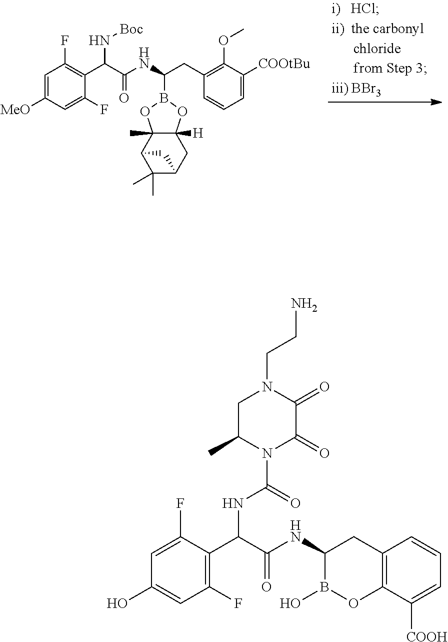

In a similar manner to the synthesis of Example 26, the title compound was prepared after reversed phase HPLC purification. ESI-MS m/z 590 (M+H)⁺.

### Example 235: (3R)-3-(2-(2,3-difluoro-4-hydroxyphenyl)-2-(4-(2-fluoroethyl)-2,3-dioxopiperazine-1-carboxamido)acetamido)-2-hydroxy-3,4-dihydro-2H-benzo[e][1,2]oxaborinine-8-carboxylic acid

### Step 1. Synthesis of tert-butyl 3-((2R)-2-(2-((tert-butoxycarbonyl)amino)-2-(2,3-difluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

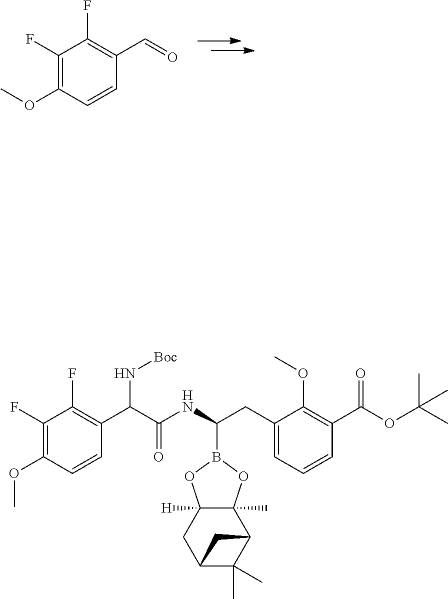 [SMILES: COc1ccc(C=O)c(F)c1F ; COc1ccc(C(NC(=O)OC(C)(C)C)C(=O)N[C@@H](Cc2cccc(C(=O)OC(C)(C)C)c2OC)B2O[C@@H]3C[C@@H]4C[C@@H](C4(C)C)[C@]3(C)O2)c(F)c1F]

By following the procedures described in Step 1-3 of Example 17, utilizing 2,3-difluoro-4-methoxybenzaldehyde in place of 4-methoxy-3-nitrobenzaldehyde in Step 1, the title compound was prepared. ESI-MS m/z 728.6 (M+1)⁺.

### Step 2. Synthesis of (3R)-3-(2-(2,3-difluoro-4-hydroxyphenyl)-2-(4-(2-fluoroethyl)-2,3-dioxopiperazine-1-carboxamido)acetamido)-2-hydroxy-3,4-dihydro-2H-benzo[e][1,2]oxaborinine-8-carboxylic acid

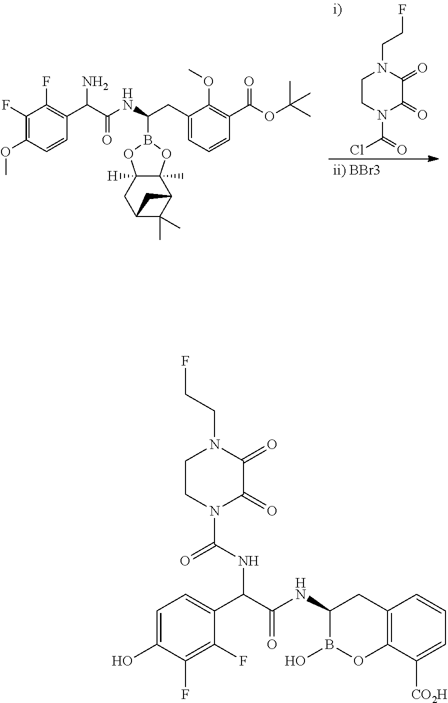 [SMILES: COc1ccc(C(N)C(=O)N[C@@H](Cc2cccc(C(=O)OC(C)(C)C)c2OC)B2O[C@@H]3C[C@@H]4C[C@@H](C4(C)C)[C@]3(C)O2)c(F)c1F ; O=C1C(=O)N(C(=O)Cl)CCN1CCF ; O=C(O)c1cccc2c1OB(O)[C@@H](NC(=O)C(NC(=O)N1CCN(CCF)C(=O)C1=O)c1ccc(O)c(F)c1F)C2]

In a similar manner to the synthesis of Example 36, utilizing the above carbonyl chloride in place of 4-ethyl-2,3-dioxopiperazine-1-carbonyl chloride for the acylation in Step 2, the title compound was prepared. ESI-MS m/z 578.6 (M+1)⁺.

### Example 236: (R)-3-((R)-2-(4-((1-aminocyclopropyl)methyl)-2,3-dioxopiperazine-1-carboxamido)-2-(2,3-difluoro-4-hydroxyphenyl)acetamido)-2-hydroxy-3,4-dihydro-2H-benzo[e][1,2]oxaborinine-8-carboxylic acid

In a similar manner to the synthesis of Example 235, utilizing tert-butyl (1-((4-(chlorocarbonyl)-2,3-dioxopiperazin-1-yl)methyl)cyclopropyl)carbamate in place of 4-(2-fluoroethyl)-2,3-dioxopiperazine 1-carbonyl chloride for the acylation in Step 2, the title compound was isolated as the first eluting peak. ESI-MS m/z 601.3 (M+1)⁺.

### Example 237: (R)-3-((S)-2-(4-((1-aminocyclopropyl)methyl)-2,3-dioxopiperazine-1-carboxamido)-2-(2,3-difluoro-4-hydroxyphenyl)acetamido)-2-hydroxy-3,4-dihydro-2H-benzo[e][1,2]oxaborinine-8-carboxylic acid

The title compound was isolated as the second eluting peak after reversed phase HPLC purification of BBr₃ reaction mixture of Example 236. ESI-MS m/z 601.3 (M+1)⁺.

### Example 238: (R)-3-((R)-2-(4-(3-aminopropyl)-2,3-dioxopiperazine-1-carboxamido)-2-(2,3-difluoro-4-hydroxyphenyl)acetamido)-2-hydroxy-3,4-dihydro-2H-benzo[e][1,2]oxaborinine-8-carboxylic acid

In a similar manner to the synthesis of Example 235, utilizing tert-butyl (3-(4-(chlorocarbonyl)-2,3-dioxopiperazin-1-yl)propyl)carbamate in place of 4-(2-fluoroethyl)-2,3-dioxopiperazine-1-carbonyl chloride for the acylation in Step 2, the title compound was isolated as the first eluting peak. ESI-MS m/z 589.8 (M+1)⁺.

### Example 239: (R)-3-((S)-2-(4-(3-aminopropyl)-2,3-dioxopiperazine-1-carboxamido)-2-(2,3-difluoro-4-hydroxyphenyl)acetamido)-2-hydroxy-3,4-dihydro-2H-benzo[e][1,2]oxaborinine-8-carboxylic acid

The title compound was isolated as the second eluting peak after reversed phase HPLC purification of BBr₃ reaction mixture of Example 238. ESI-MS m/z 589.8 (M+1)⁺.

### Example 240: (3R)-3-(2-(2,3-difluoro-4-hydroxyphenyl)-2-((S)-4-ethyl-6-methyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 17, utilizing 2-((tert-butoxycarbonyl)amino)-2-(2,3-difluoro-4-methoxyphenyl)acetic acid in place of 2-((tert-butoxycarbonyl)amino)-2-(4-methoxy-3-nitrophenyl)acetic acid. ESI-MS m/z 575 (MH)⁺.

### Example 241: (3R)-3-(2-(2,3-difluoro-4-hydroxyphenyl)-2-((S)-4-(2-fluoroethyl)-6-methyl-2,3-dioxopiperazine-1-carboxamido)acetamido)-2-hydroxy-3,4-dihydro-2H-benzo[e][1,2]oxaborinine-8-carboxylic acid

In a similar manner to the synthesis of Example 235, utilizing (S)-4-(2-fluoroethyl)-6-methyl-2,3-dioxopiperazine-1-carbonyl chloride in place of 4-(2-fluoroethyl)-2,3-dioxopiperazine-1-carbonyl chloride for the acylation in Step 2, the title compound was prepared. ESI-MS m/z 592.6 (M+1)⁺.

### Example 242: (3R)-3-(2-((S)-4-(2-aminoethyl)-6-methyl-2,3-dioxopiperazine-1-carboxamido)-2-(2,3-difluoro-4-hydroxyphenyl)acetamido)-2-hydroxy-3,4-dihydro-2H-benzo[e][1,2]oxaborinine-8-carboxylic acid

In a similar manner to the synthesis of Example 235, utilizing tert-butyl (S)-(2-(4-(chlorocarbonyl)-5-methyl-2,3-dioxopiperazin-1-yl)ethyl)carbamate in place of 4-(2-fluoroethyl)-2,3-dioxopiperazine-1-carbonyl chloride for the acylation in Step 2, the title compound was prepared. ESI-MS m/z 592.6 (M+1)⁺.

### Example 243: (3R)-3-(2-((S)-4-(3-aminopropyl)-6-methyl-2,3-dioxopiperazine-1-carboxamido)-2-(2,3-difluoro-4-hydroxyphenyl)acetamido)-2-hydroxy-3,4-dihydro-2H-benzo[e][1,2]oxaborinine-8-carboxylic acid

The title compound was prepared in a similar manner to the synthesis of Example 17, utilizing 2-((tert-butoxycarbonyl)amino)-2-(2,3-difluoro-4-methoxyphenyl)acetic acid in place of 2-((tert-butoxycarbonyl)amino)-2-(4-methoxy-3-nitrophenyl)acetic acid. ESI-MS m/z 604 (MH)⁺.

### Example 244: (R)-3-((R)-2-(4-((1-aminocyclopropyl)methyl)-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2,3,5-trifluoro-4-methoxyphenyl)acetic acid

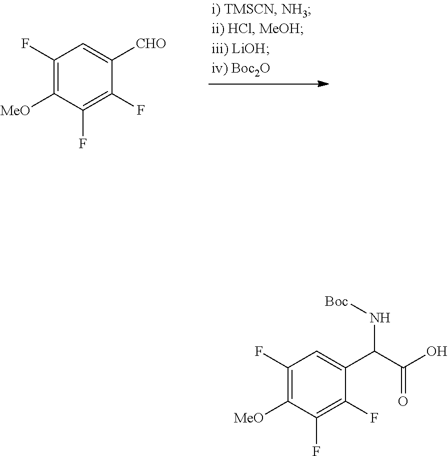 [SMILES: COc1c(F)cc(C=O)c(F)c1F ; COc1c(F)cc(C(NC(=O)OC(C)(C)C)C(=O)O)c(F)c1F]

In a similar manner to the synthesis of Step 1 of Example 17, the title compound was prepared from 2,3,5-trifluoro-4-methoxybenzaldehyde. ESI-MS m/z 358 (M+Na)⁺.

### Step 2. Synthesis of tert-butyl 3-((2R)-2-(2-((tert-butoxycarbonyl)amino)-2-(2,3,5-trifluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

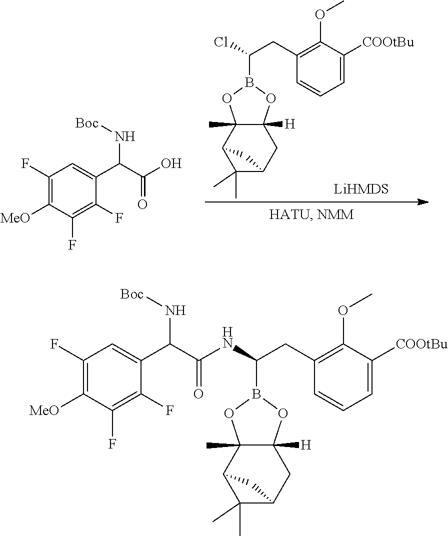

By following the General coupling method C, the title compound was prepared from the above acid and the chloride intermediate (prepared as previous reported, WO 2014/089365). ESI-MS m/z 747 (M+H)⁺.

### Step 3. Synthesis of tert-butyl (1-((4-(chlorocarbonyl)-2,3-dioxopiperazin-1-yl)methyl)cyclopropyl)carbamate

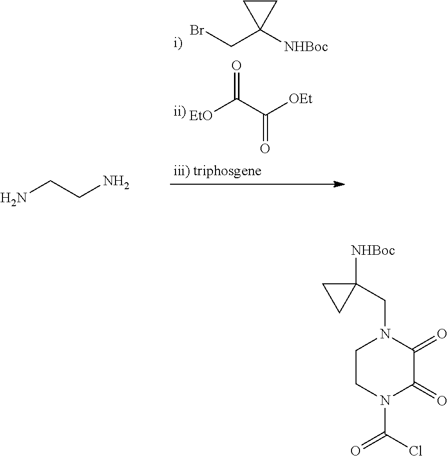

By following the same procedures as described for the synthesis of tert-butyl (1-((4-(chlorocarbonyl)-2,3-dioxopiperazin-1-yl)methyl)cyclopropyl)carbamate in Step 1 of Example 26, the title compound was prepared from tert-butyl (1-(bromomethyl)cyclopropyl)carbamate.

### Step 4. Synthesis of (R)-3-((R)-2-(4-((1-aminocyclopropyl)methyl)-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

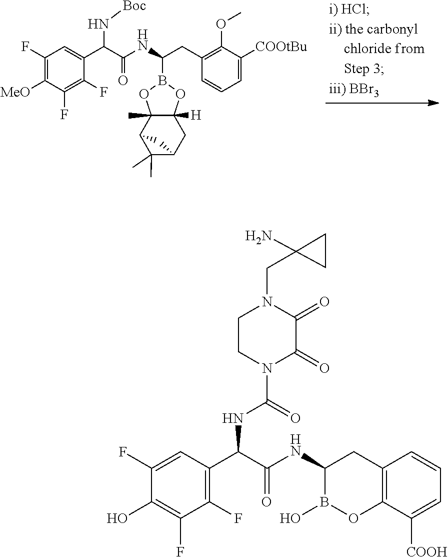

In a similar manner to the synthesis of Example 26, the title compound was prepared after reversed phase HPLC purification. isolated as the second eluting peak. ESI-MS m/z 620 (M+H)⁺.

### Example 245: (R)-3-((R)-2-(4-((1-aminocyclopropyl)methyl)-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

In a same manner to the synthesis of Example 244, the title compound was prepared after reversed phase HPLC purification. isolated as the first eluting peak. ESI-MS m/z 620 (M+H)⁺.

### Example 246: (R)-3-((R)-2-(4-(3-aminopropyl)-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2,3,5-trifluoro-4-methoxyphenyl)acetic acid

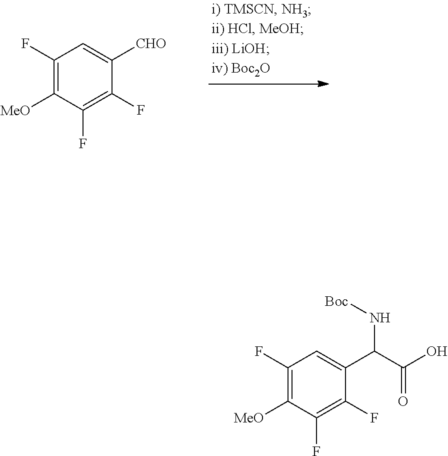 [SMILES: COc1c(F)cc(C=O)c(F)c1F ; COc1c(F)cc(C(NC(=O)OC(C)(C)C)C(=O)O)c(F)c1F]

In a similar manner to the synthesis of Step 1 of Example 17, the title compound was prepared from 2,3,5-trifluoro-4-methoxybenzaldehyde. ESI-MS m/z 358 (M+Na)⁺.

### Step 2. Synthesis of tert-butyl 3-((2R)-2-(2-((tert-butoxycarbonyl)amino)-2-(2,3,5-trifluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

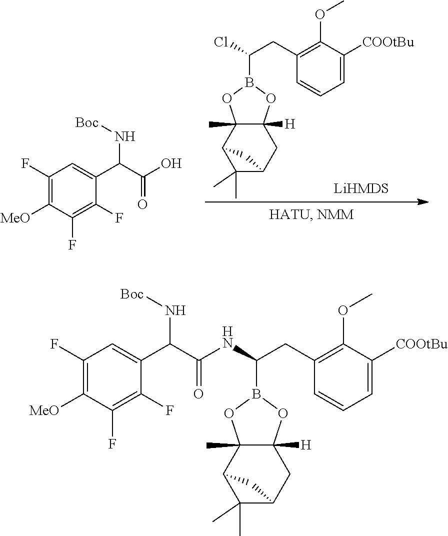

By following the General coupling method C, the title compound was prepared from the above acid and the chloride intermediate (prepared as previous reported, WO 2014/089365). ESI-MS m/z 747 (M+H).

### Step 3. Synthesis of tert-butyl (3-(4-(chlorocarbonyl)-2,3-dioxopiperazin-1-yl)propyl)carbamate

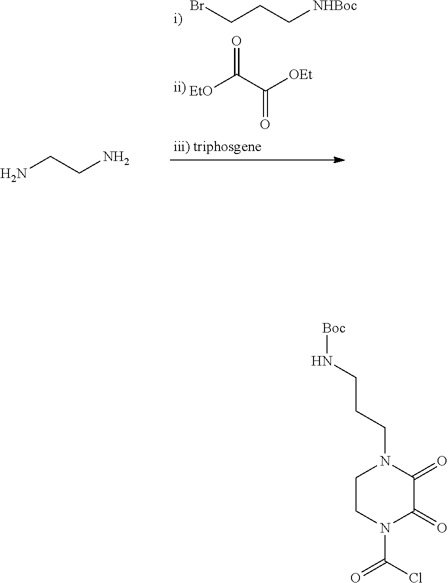

By following the same procedures as described for the synthesis of tert-butyl (3-(4-(chlorocarbonyl)-2,3-dioxopiperazin-1-yl)propyl)carbamate in Step 1 of Example 26, the title compound was prepared from tert-butyl (3-bromopropyl)carbamate.

### Step 4. Synthesis of (R)-3-((R)-2-(4-(3-aminopropyl)-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

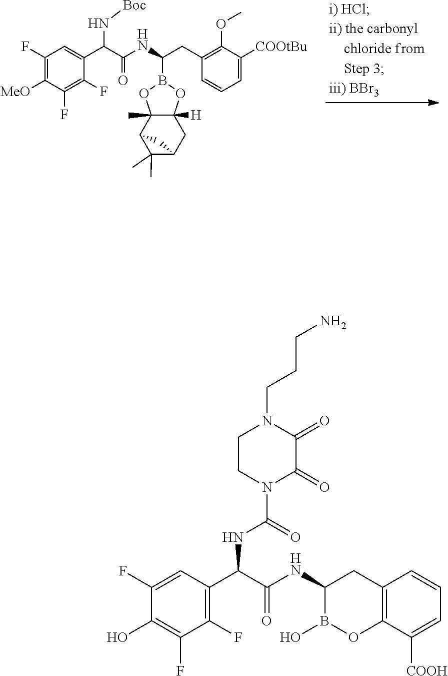

In a similar manner to the synthesis of Example 26, the title compound was prepared after reversed phase HPLC purification, isolated as the first eluting peak. ESI-MS m/z 608 (M+H)⁺.

### Example 247: (R)-3-((S)-2-(4-(3-aminopropyl)-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

In a same manner to the synthesis of Example 246, the title compound was prepared after reversed phase HPLC purification, isolated as the second eluting peak. ESI-MS m/z 608 (M+H)⁺.

### Example 248: (3R)-3-(2-((S)-4-(2-fluoroethyl)-6-methyl-2,3-dioxopiperazine-1-carboxamido)-2-(2,3,6-trifluoro-4-hydroxyphenyl)acetamido)-2-hydroxy-3,4-dihydro-2H-benzo[e][1,2]oxaborinine-8-carboxylic acid

### Step 1. Synthesis of 2-((tert-butoxycarbonyl)amino)-2-(2,3,5-trifluoro-4-methoxyphenyl)acetic acid

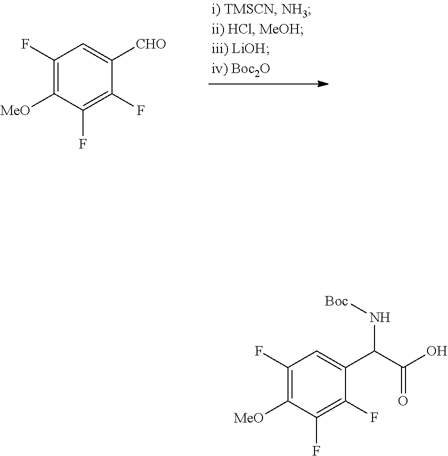 [SMILES: COc1c(F)cc(C=O)c(F)c1F ; COc1c(F)cc(C(NC(=O)OC(C)(C)C)C(=O)O)c(F)c1F]

In a similar manner to the synthesis of Step 1 of Example 17, the title compound was prepared from 2,3,5-trifluoro-4-methoxybenzaldehyde. ESI-MS m/z 358 (M+Na)⁺.

### Step 2. Synthesis of tert-butyl 3-((2R)-2-(2-((tert-butoxycarbonyl)amino)-2-(2,3,5-trifluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

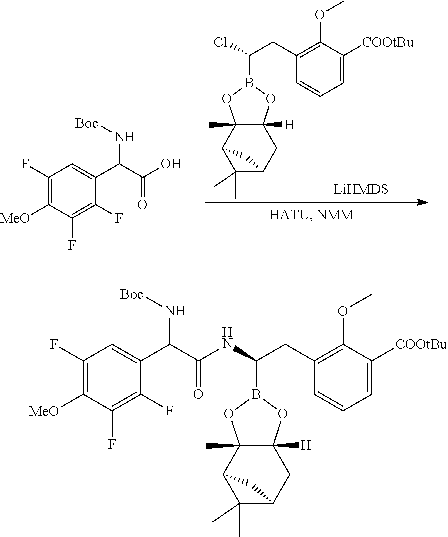

By following the General coupling method C, the title compound was prepared from the above acid and the chloride intermediate (prepared as previous reported, WO 2014/089365). ESI-MS m/z 747 (M+H)⁺.

### Step 3. Synthesis of (S)-4-(2-fluoroethyl)-6-methyl-2,3-dioxopiperazine-1-carbonyl chloride

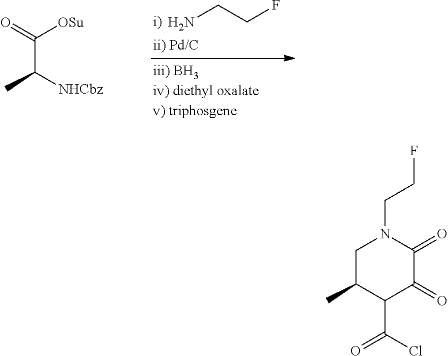

By following the same procedure as described for the Synthesis of tert-butyl (S)-(2-(4-(chloroarbonyl)-5-methyl-2,3-dioxopiperazin-1-yl)ethyl)carbamate in Step 1 of Example 234, the title compound was prepared from 2-fluoroethan-1-amine.

### Step 4. Synthesis of (3R)-3-(2-((S)-4-(2-fluoroethyl)-6-methyl-2,3-dioxopiperazine-1-carboxamido)-2-(2,3,5-trifluoro-4-hydroxyphenyl)acetamido)-2-hydroxy-3,4-dihydro-2H-benzo[e][1,2]oxaborinine-8-carboxylic acid

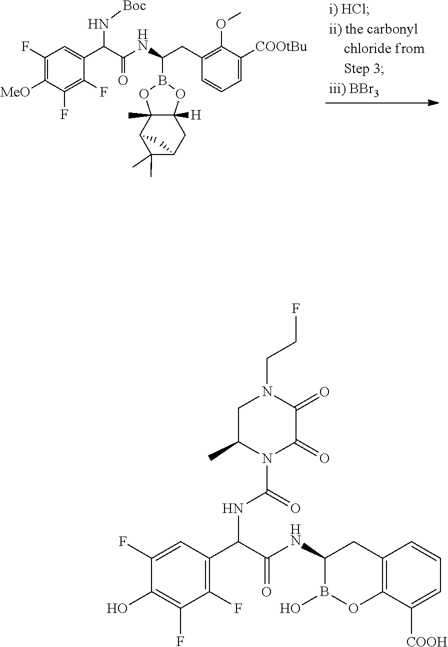

In a similar manner to the synthesis of Example 26, the title compound was prepared after reversed phase HPLC purification. ESI-MS m/z 611 (M+H)⁺.

### Example 249: (3R)-3-(2-(4-(2-bromoethyl)-2,3-dioxopiperazine-1-carboxamido)-2-(2,3-difluoro-4-hydroxyphenyl)acetamido)-2-hydroxy-3,4-dihydro-2H-benzo[e][1,2]oxaborinine-8-carboxylic acid

The title compound was isolated as a byproduct after reversed phase HPLC purification of BBr₃ reaction mixture of Example 235. ESI-MS m/z 638.4/640.4 (MH/MH+2)⁺.

### Example 250: (3R)-3-(2-(4-ethyl-2,3-dioxopiperazine-1-carboxamido)-2-(4-phosphonophenyl)acetamido)-2-hydroxy-3,4-dihydro-2H-benzo[e][1,2]oxaborinine-8-carboxylic acid

### Step 1: Synthesis of tert-butyl 3-((2R)-2-(2-(4-(bis(benzyloxy)phosphoryl)phenyl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

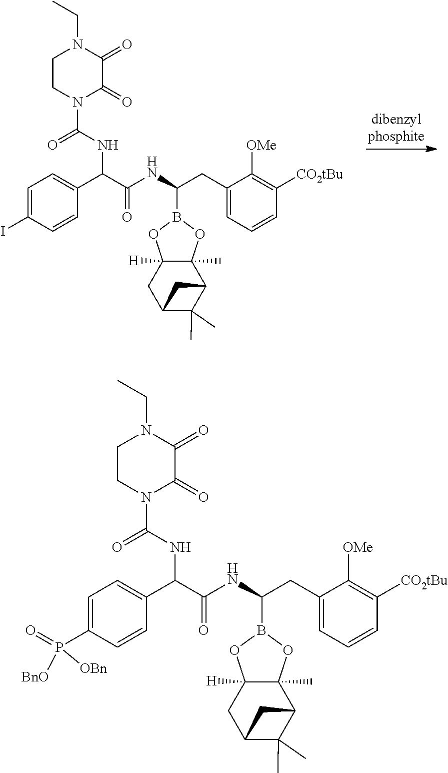 [SMILES: CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ccc(I)cc2)C(=O)C1=O ; CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ccc(P(=O)(OCc3ccccc3)OCc3ccccc3)cc2)C(=O)C1=O]

To tert-butyl 3-((2R)-2-(2-(4-ethyl-2,3-dioxopiperazine-1-carboxamido)-2-(4-iodophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate 0.25 g (0.29 mmol) was added diisopropylethylamine 0.153 mL (0.88 mmol, 3 eq), tetrakis(triphenylphosphine)palladium(0) 0.034 g, (0.03 mmol, 10 mol %), dibenzyl phosphite 0.13 mL (0.58 mmol, 2 eq), followed by 1-methyl-2-pyrrolidinone (5 mL) and degassed 3× under argon. The mixture was stirred at 90° C. for 1 h, cooled, diluted with ethyl acetate, washed with water 3×, dried over sodium sulfate, and concentrated to give the title compound. ESI-MS m/z 991 (MH)⁺.

### Step 2: Synthesis of (3R)-3-(2-(4-ethyl-2,3-dioxopiperazine-1-carboxamido)-2-(4-phosphonophenyl)acetamido)-2-hydroxy-3,4-dihydro-2H-benzo[e][1,2]oxaborinine-8-carboxylic acid

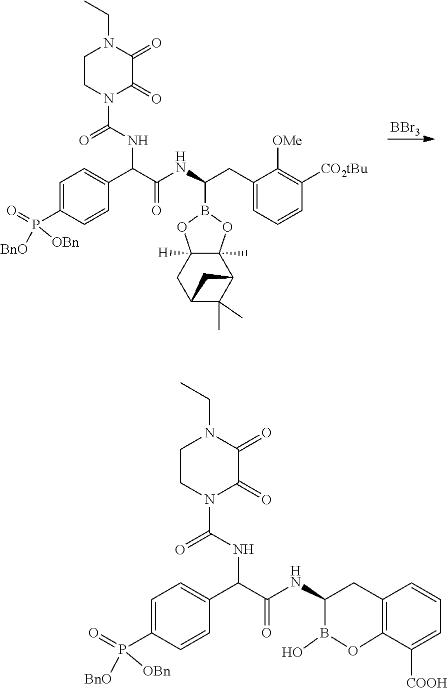 [SMILES: CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ccc(P(=O)(OCc3ccccc3)OCc3ccccc3)cc2)C(=O)C1=O ; "BBr3" ; CCN1CCN(C(=O)NC(C(=O)N[C@H]2Cc3cccc(C(=O)O)c3OB2O)c2ccc(P(=O)(OCc3ccccc3)OCc3ccccc3)cc2)C(=O)C1=O]

By following the general deprotection and purification method, the above compound was treated with excess BBr₃ to yield the title compound after reversed phase HPLC purification. ESI-MS m/z 589 (MH)⁺.

### Example 251: (3R)-3-(2-(2-aminothiazole-4-carboxamido)-2-(2,6-difluoro-4-hydroxyphenyl)acetamido)-2-hydroxy-3,4-dihydro-2H-benzo[e][1,2]oxaborinine-8-carboxylic acid

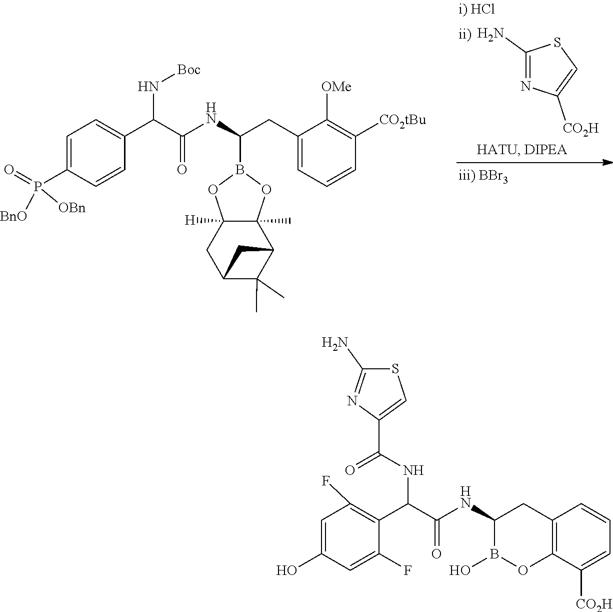 [SMILES: COc1c(C[C@H](NC(=O)C(NC(=O)OC(C)(C)C)c2ccc(P(=O)(OCc3ccccc3)OCc3ccccc3)cc2)B2O[C@@H]3C[C@@H]4C[C@@H](C4(C)C)[C@]3(C)O2)cccc1C(=O)OC(C)(C)C ; Nc1nc(C(=O)O)cs1 ; Nc1nc(C(=O)NC(C(=O)N[C@H]2Cc3cccc(C(=O)O)c3OB2O)c2c(F)cc(O)cc2F)cs1]

A solution of tert-butyl 3-((2R)-2-(2-((tert-butoxycarbonyl)amino)-2-(2,6-difluoro-4-methoxyphenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate was deprotected in the same manner as Example 17. To a solution of crude product (100 mg, 0.150 mmol) in DMF (1.5 mL) was added 2-aminothiazole-4-carboxylic acid (24 mg, 0.165 mmol), HATU (69 mg, 0.180 mmol), and DIPEA (52 μL, 0.300 mmol) sequentially. The mixture was stirred at room temperature overnight then quenched with water. The layers were separated and the aq. layer was extracted with ethyl acetate (3×20 mL). The combined organic layers were dried (Na₂SO₄), filtered, and concentrated. The title compound was prepared in a similar manner as Example 17. ESI-MS m/z 519 (M+H)⁺.

TABLE 1
Example compounds.

| | | | ESI-MS (m/z) for |
|---|---|---|---|
| Ex. | Structure | MW | [MH]⁺ |
| 1 | 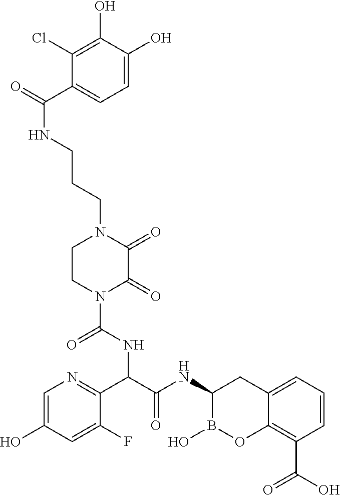 [SMILES: O=C(NCCCN1CCN(C(=O)NC(C(=O)N[C@H]2Cc3cccc(C(=O)O)c3OB2O)c2ncc(O)cc2F)C(=O)C1=O)c1ccc(O)c(O)c1Cl] | 742.86 | 743.2 |
| 2 | 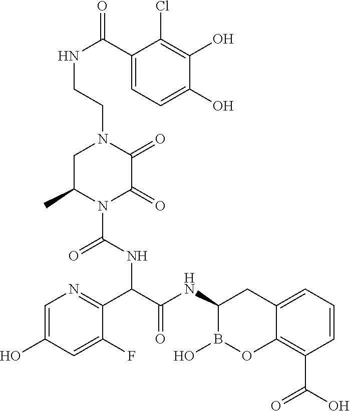 [SMILES: C[C@H]1CN(CCNC(=O)c2ccc(O)c(O)c2Cl)C(=O)C(=O)N1C(=O)NC(C(=O)N[C@H]1Cc2cccc(C(=O)O)c2OB1O)c1ncc(O)cc1F] | 742.86 | 743.1 |
| 3 | 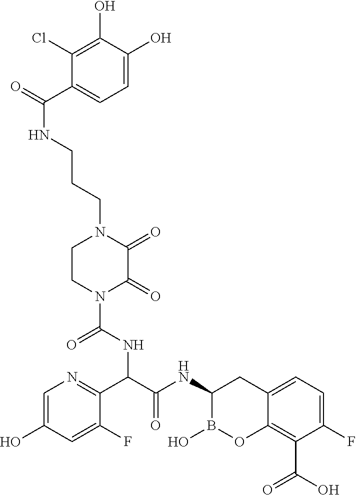 [SMILES: O=C(NCCCN1CCN(C(=O)NC(C(=O)N[C@H]2Cc3ccc(F)c(C(=O)O)c3OB2O)c2ncc(O)cc2F)C(=O)C1=O)c1ccc(O)c(O)c1Cl] | 760.85 | 761.2 |
| 4 | 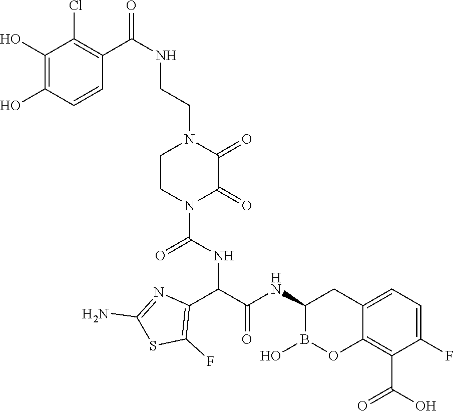 [SMILES: Nc1nc(C(NC(=O)N2CCN(CCNC(=O)c3ccc(O)c(O)c3Cl)C(=O)C2=O)C(=O)N[C@H]2Cc3ccc(F)c(C(=O)O)c3OB2O)c(F)s1] | 751.86 | 752.1 |
| 5 | 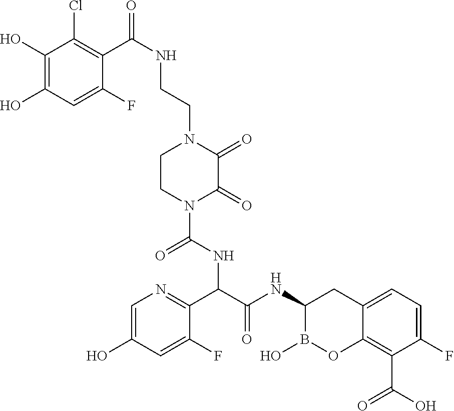 [SMILES: O=C(NCCN1CCN(C(=O)NC(C(=O)N[C@H]2Cc3ccc(F)c(C(=O)O)c3OB2O)c2ncc(O)cc2F)C(=O)C1=O)c1c(F)cc(O)c(O)c1Cl] | 764.82 | 765.1 |
| 6 | 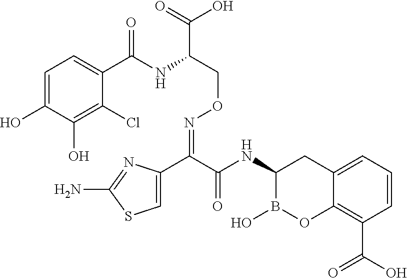 [SMILES: Nc1nc(/C(=N/OC[C@H](NC(=O)c2ccc(O)c(O)c2Cl)C(=O)O)C(=O)N[C@H]2Cc3cccc(C(=O)O)c3OB2O)cs1] | 633.78 | 634.0 |
| 7 | 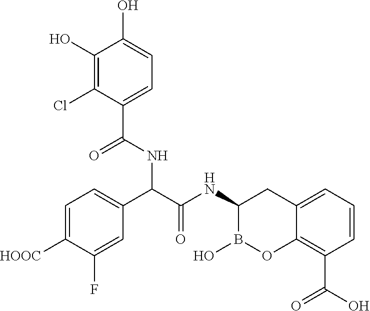 [SMILES: O=C(O)c1ccc(C(NC(=O)c2ccc(O)c(O)c2Cl)C(=O)N[C@H]2Cc3cccc(C(=O)O)c3OB2O)cc1F] | 572.69 | 573.1 |
| 8 | 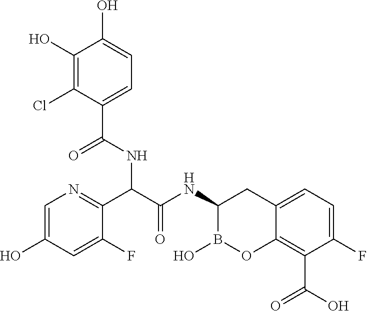 [SMILES: O=C(NC(C(=O)N[C@H]1Cc2ccc(F)c(C(=O)O)c2OB1O)c1ncc(O)cc1F)c1ccc(O)c(O)c1Cl] | 563.66 | 564 |
| 9 | 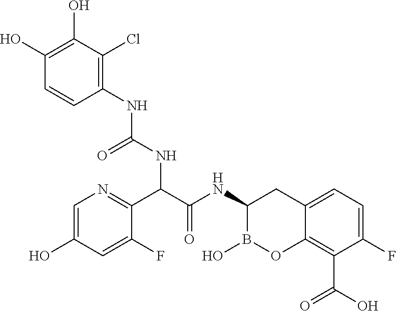 [SMILES: O=C(Nc1ccc(O)c(O)c1Cl)NC(C(=O)N[C@H]1Cc2ccc(F)c(C(=O)O)c2OB1O)c1ncc(O)cc1F] | 578.67 | 579 |
| 10 | 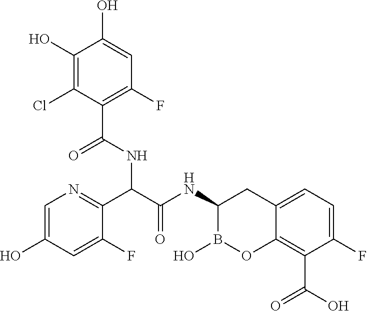 [SMILES: O=C(NC(C(=O)N[C@H]1Cc2ccc(F)c(C(=O)O)c2OB1O)c1ncc(O)cc1F)c1c(F)cc(O)c(O)c1Cl] | 581.65 | 582.1 |
| 11 | 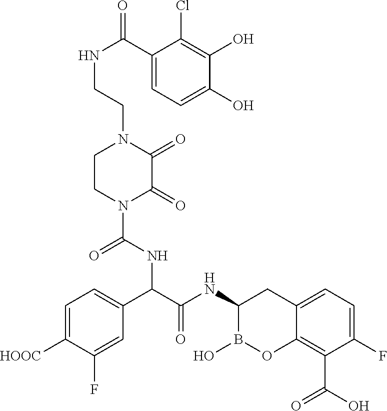 [SMILES: O=C(O)c1ccc(C(NC(=O)N2CCN(CCNC(=O)c3ccc(O)c(O)c3Cl)C(=O)C2=O)C(=O)N[C@H]2Cc3ccc(F)c(C(=O)O)c3OB2O)cc1F] | 773.85 | 774.1 |
| 12 | 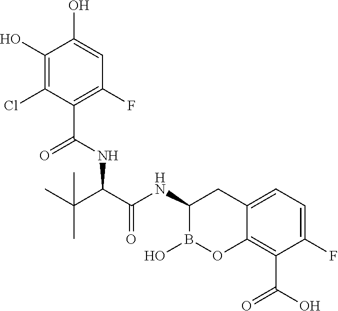 [SMILES: CC(C)(C)[C@@H](NC(=O)c1c(F)cc(O)c(O)c1Cl)C(=O)N[C@H]1Cc2ccc(F)c(C(=O)O)c2OB1O] | 526.68 | 527.1 |
| 13 | 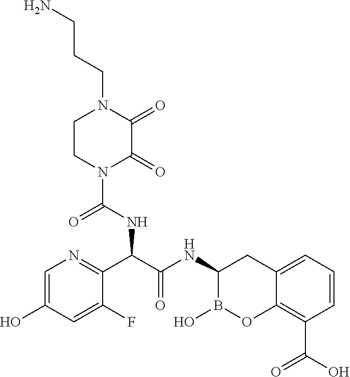 [SMILES: NCCCN1CCN(C(=O)N[C@@H](C(=O)N[C@H]2Cc3cccc(C(=O)O)c3OB2O)c2ncc(O)cc2F)C(=O)C1=O] | 572.31 | 573.1 |
| 14 | 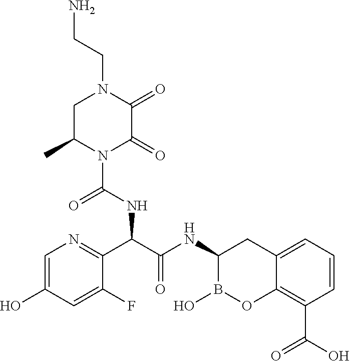 [SMILES: C[C@H]1CN(CCN)C(=O)C(=O)N1C(=O)N[C@@H](C(=O)N[C@H]1Cc2cccc(C(=O)O)c2OB1O)c1ncc(O)cc1F] | 572.31 | 573.2 |
| 15 | 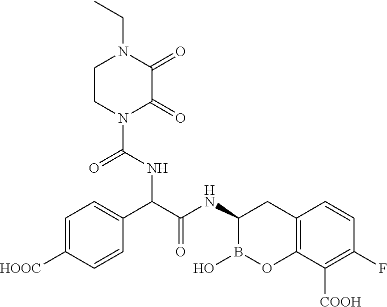 [SMILES: CCN1CCN(C(=O)NC(C(=O)N[C@H]2Cc3ccc(F)c(C(=O)O)c3OB2O)c2ccc(C(=O)O)cc2)C(=O)C1=O] | 570.29 | 571 |
| 16 | 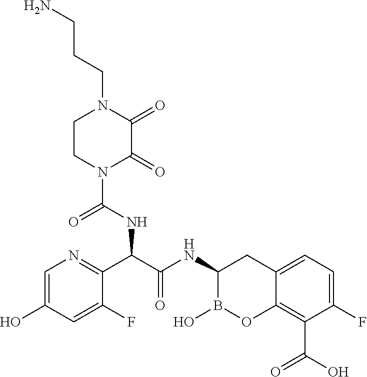 [SMILES: NCCCN1CCN(C(=O)N[C@@H](C(=O)N[C@H]2Cc3ccc(F)c(C(=O)O)c3OB2O)c2ncc(O)cc2F)C(=O)C1=O] | 590.30 | 591.2 |
| 17 | 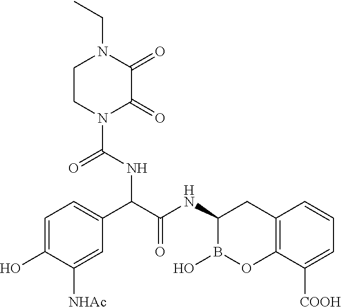 [SMILES: CCN1CCN(C(=O)NC(C(=O)N[C@H]2Cc3cccc(C(=O)O)c3OB2O)c2ccc(O)c(NC(C)=O)c2)C(=O)C1=O] | 581.35 | 582 |
| 18 | 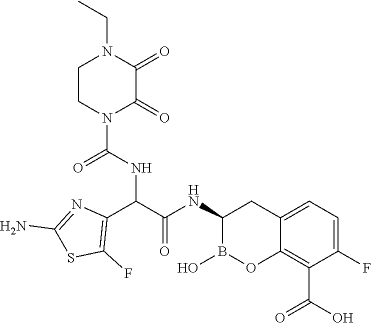 [SMILES: CCN1CCN(C(=O)NC(C(=O)N[C@H]2Cc3ccc(F)c(C(=O)O)c3OB2O)c2nc(N)sc2F)C(=O)C1=O] | 566.30 | 567.1 |
| 19 | 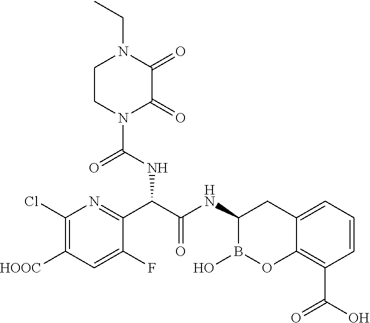 [SMILES: CCN1CCN(C(=O)N[C@H](C(=O)N[C@H]2Cc3cccc(C(=O)O)c3OB2O)c2nc(Cl)c(C(=O)O)cc2F)C(=O)C1=O] | 605.72 | 606.1 |
| 20 | 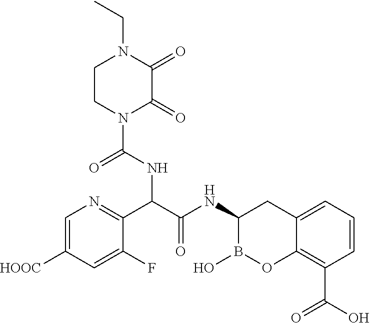 [SMILES: CCN1CCN(C(=O)NC(C(=O)N[C@H]2Cc3cccc(C(=O)O)c3OB2O)c2ncc(C(=O)O)cc2F)C(=O)C1=O] | 571.28 | 572.1 |
| 21 | 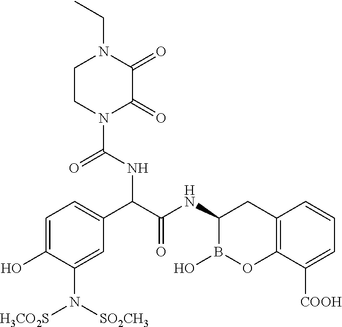 | 695.48 | 696 |
| 22 | 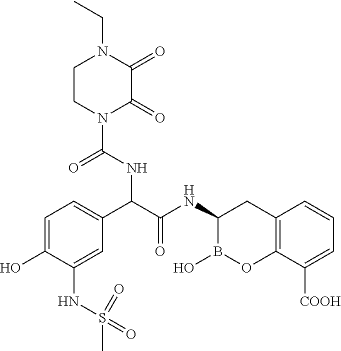 [SMILES: CCN1CCN(C(=O)NC(C(=O)N[C@H]2Cc3cccc(C(=O)O)c3OB2O)c2ccc(O)c(NS(C)(=O)=O)c2)C(=O)C1=O] | 617.39 | 618 |
| 23 | 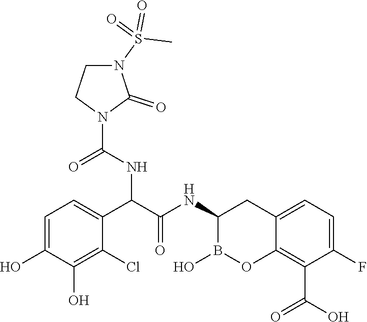 [SMILES: CS(=O)(=O)N1CCN(C(=O)NC(C(=O)N[C@H]2Cc3ccc(F)c(C(=O)O)c3OB2O)c2ccc(O)c(O)c2Cl)C1=O] | 614.75 | 615.1 |
| 24 | 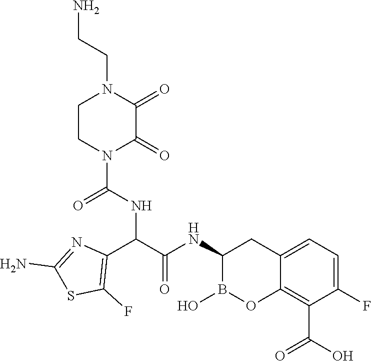 [SMILES: NCCN1CCN(C(=O)NC(C(=O)N[C@H]2Cc3ccc(F)c(C(=O)O)c3OB2O)c2nc(N)sc2F)C(=O)C1=O] | 581.31 | 582.1 |
| 25 | 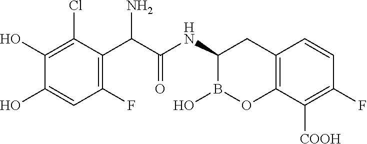 [SMILES: NC(C(=O)N[C@H]1Cc2ccc(F)c(C(=O)O)c2OB1O)c1c(F)cc(O)c(O)c1Cl] | 442.56 | 443 |
| 26 | 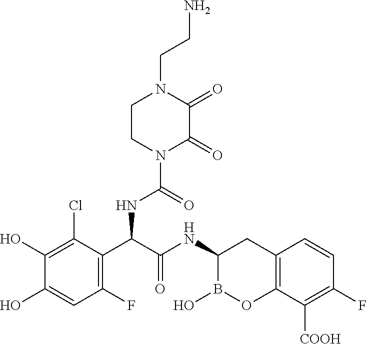 [SMILES: NCCN1CCN(C(=O)N[C@@H](C(=O)N[C@H]2Cc3ccc(F)c(C(=O)O)c3OB2O)c2c(F)cc(O)c(O)c2Cl)C(=O)C1=O] | 625.73 | 626 |
| 27 | 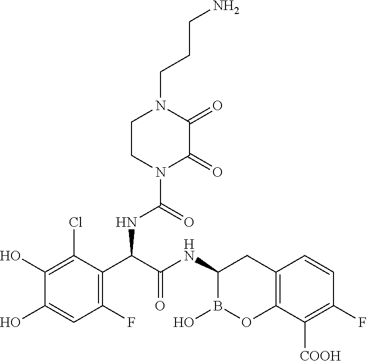 [SMILES: NCCCN1CCN(C(=O)N[C@@H](C(=O)N[C@H]2Cc3ccc(F)c(C(=O)O)c3OB2O)c2c(F)cc(O)c(O)c2Cl)C(=O)C1=O] | 639.76 | 620 |
| 28 | 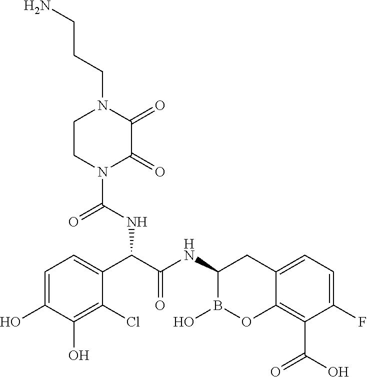 [SMILES: NCCCN1CCN(C(=O)N[C@H](C(=O)N[C@H]2Cc3ccc(F)c(C(=O)O)c3OB2O)c2ccc(O)c(O)c2Cl)C(=O)C1=O] | 621.77 | 622.2 |
| 29 | 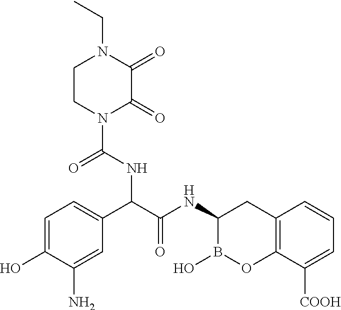 [SMILES: CCN1CCN(C(=O)NC(C(=O)N[C@H]2Cc3cccc(C(=O)O)c3OB2O)c2ccc(O)c(N)c2)C(=O)C1=O] | 539.31 | 540 |
| 30 | 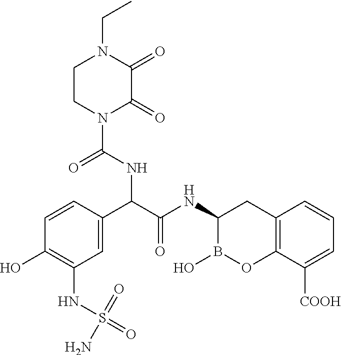 [SMILES: CCN1CCN(C(=O)NC(C(=O)N[C@H]2Cc3cccc(C(=O)O)c3OB2O)c2ccc(O)c(NS(N)(=O)=O)c2)C(=O)C1=O] | 618.38 | 619 |
| 31 | 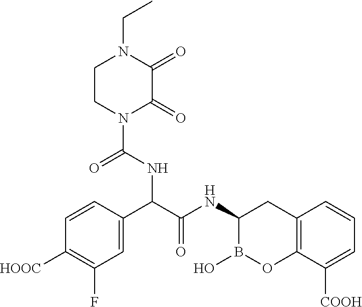 [SMILES: CCN1CCN(C(=O)NC(C(=O)N[C@H]2Cc3cccc(C(=O)O)c3OB2O)c2ccc(C(=O)O)c(F)c2)C(=O)C1=O] | 570.29 | 571 |
| 32 | 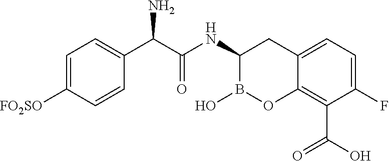 | 456.18 | 457 |
| 33 | 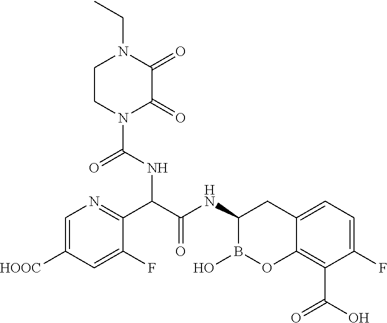 [SMILES: CCN1CCN(C(=O)NC(C(=O)N[C@H]2Cc3ccc(F)c(C(=O)O)c3OB2O)c2ncc(C(=O)O)cc2F)C(=O)C1=O] | 589.27 | 590.1 |
| 34 | 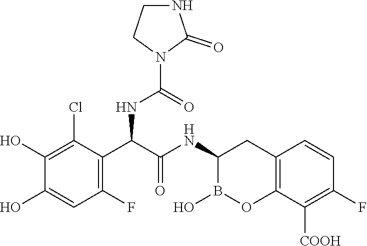 [SMILES: O=C(O)c1c(F)ccc2c1OB(O)[C@@H](NC(=O)[C@H](NC(=O)N1CCNC1=O)c1c(F)cc(O)c(O)c1Cl)C2] | 554.65 | 555 |
| 35 | 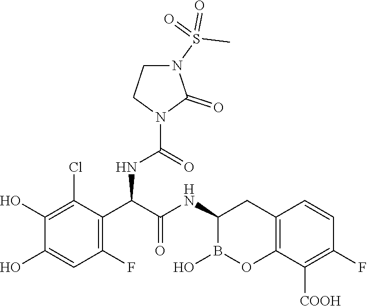 [SMILES: CS(=O)(=O)N1CCN(C(=O)N[C@@H](C(=O)N[C@H]2Cc3ccc(F)c(C(=O)O)c3OB2O)c2c(F)cc(O)c(O)c2Cl)C1=O] | 632.74 | 633 |
| 36 | 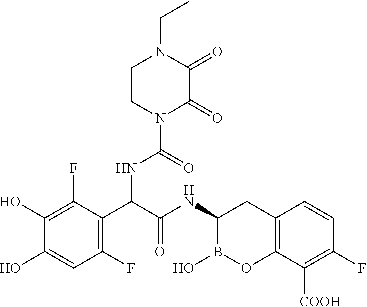 [SMILES: CCN1CCN(C(=O)NC(C(=O)N[C@H]2Cc3ccc(F)c(C(=O)O)c3OB2O)c2c(F)cc(O)c(O)c2F)C(=O)C1=O] | 594.26 | 595 |
| 37 | 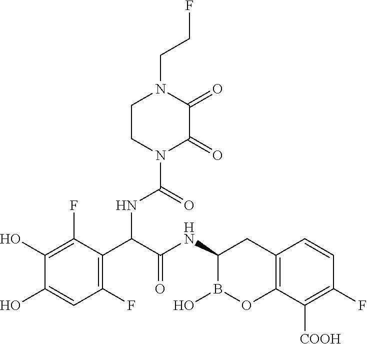 [SMILES: O=C(O)c1c(F)ccc2c1OB(O)[C@@H](NC(=O)C(NC(=O)N1CCN(CCF)C(=O)C1=O)c1c(F)cc(O)c(O)c1F)C2] | 612.25 | 613 |
| 38 | 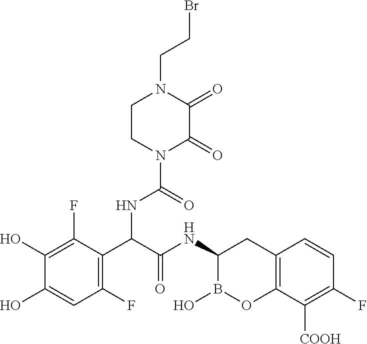 [SMILES: O=C(O)c1c(F)ccc2c1OB(O)[C@@H](NC(=O)C(NC(=O)N1CCN(CCBr)C(=O)C1=O)c1c(F)cc(O)c(O)c1F)C2] | 673.16 | 674 |
| 39 | 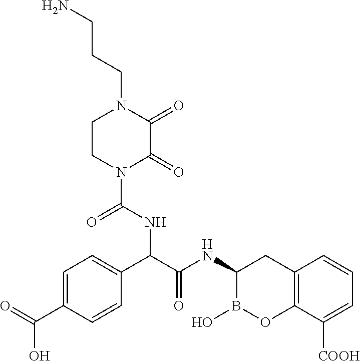 [SMILES: NCCCN1CCN(C(=O)NC(C(=O)N[C@H]2Cc3cccc(C(=O)O)c3OB2O)c2ccc(C(=O)O)cc2)C(=O)C1=O] | 581.35 | 582 |
| 40 | 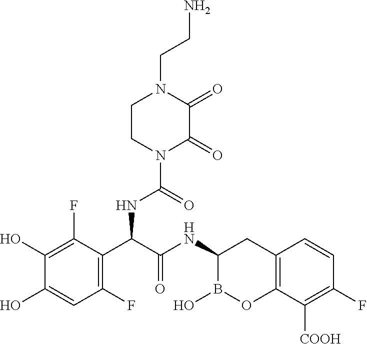 [SMILES: NCCN1CCN(C(=O)N[C@@H](C(=O)N[C@H]2Cc3ccc(F)c(C(=O)O)c3OB2O)c2c(F)cc(O)c(O)c2F)C(=O)C1=O] | 609.28 | 610 |
| 41 | 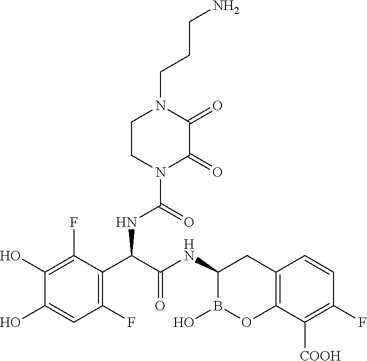 [SMILES: NCCCN1CCN(C(=O)N[C@@H](C(=O)N[C@H]2Cc3ccc(F)c(C(=O)O)c3OB2O)c2c(F)cc(O)c(O)c2F)C(=O)C1=O] | 623.31 | 624 |
| 42 | 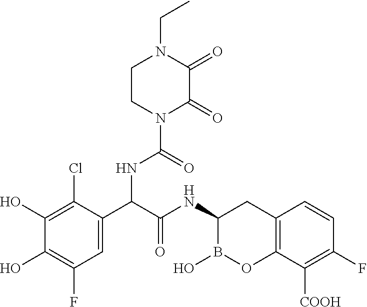 [SMILES: CCN1CCN(C(=O)NC(C(=O)N[C@H]2Cc3ccc(F)c(C(=O)O)c3OB2O)c2cc(F)c(O)c(O)c2Cl)C(=O)C1=O] | 610.71 | 611 |
| 43 | 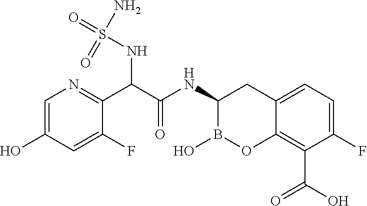 [SMILES: NS(=O)(=O)NC(C(=O)N[C@H]1Cc2ccc(F)c(C(=O)O)c2OB1O)c1ncc(O)cc1F] | 472.18 | 473 |
| 44 | 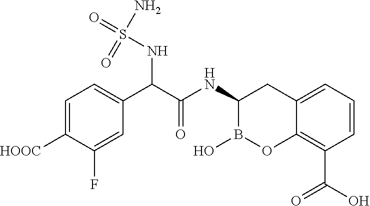 [SMILES: NS(=O)(=O)NC(C(=O)N[C@H]1Cc2cccc(C(=O)O)c2OB1O)c1ccc(C(=O)O)c(F)c1] | 481.21 | 482.1 |
| 45 | 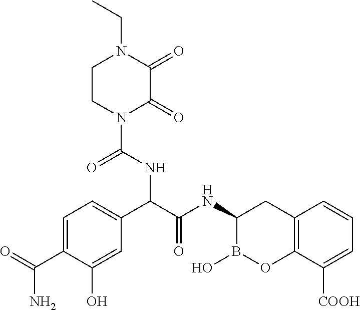 [SMILES: CCN1CCN(C(=O)NC(C(=O)N[C@H]2Cc3cccc(C(=O)O)c3OB2O)c2ccc(C(N)=O)c(O)c2)C(=O)C1=O] | 567.32 | 568 |
| 46 | 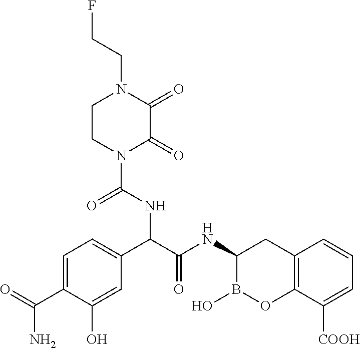 [SMILES: NC(=O)c1ccc(C(NC(=O)N2CCN(CCF)C(=O)C2=O)C(=O)N[C@H]2Cc3cccc(C(=O)O)c3OB2O)cc1O] | 585.31 | 586 |
| 47 | 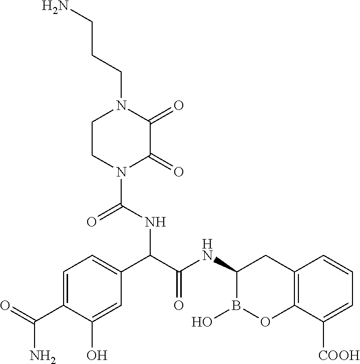 [SMILES: NCCCN1CCN(C(=O)NC(C(=O)N[C@H]2Cc3cccc(C(=O)O)c3OB2O)c2ccc(C(N)=O)c(O)c2)C(=O)C1=O] | 596.36 | 597 |
| 48 | 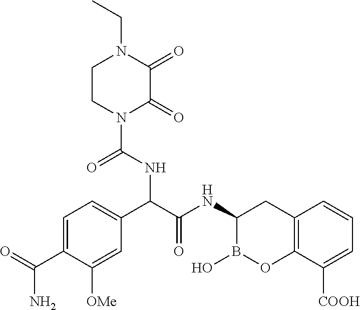 [SMILES: CCN1CCN(C(=O)NC(C(=O)N[C@H]2Cc3cccc(C(=O)O)c3OB2O)c2ccc(C(N)=O)c(OC)c2)C(=O)C1=O] | 582.33 | 583 |
| 49 | 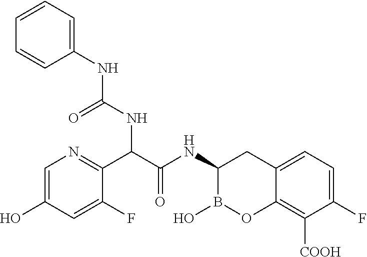 [SMILES: O=C(Nc1ccccc1)NC(C(=O)N[C@H]1Cc2ccc(F)c(C(=O)O)c2OB1O)c1ncc(O)cc1F] | 512.23 | 513.1 |
| 50 | 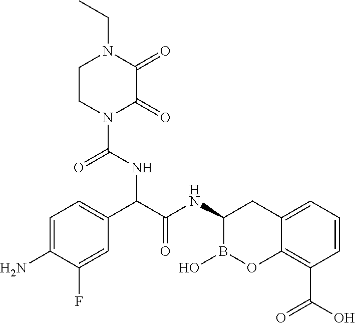 [SMILES: CCN1CCN(C(=O)NC(C(=O)N[C@H]2Cc3cccc(C(=O)O)c3OB2O)c2ccc(N)c(F)c2)C(=O)C1=O] | 541.30 | 542.2 |
| 51 | 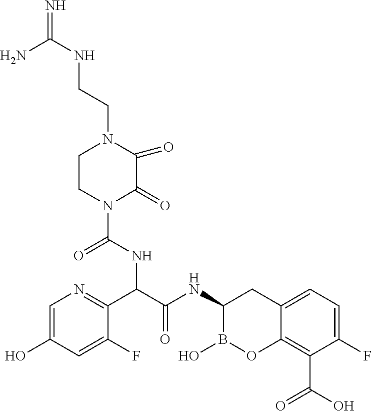 [SMILES: N=C(N)NCCN1CCN(C(=O)NC(C(=O)N[C@H]2Cc3ccc(F)c(C(=O)O)c3OB2O)c2ncc(O)cc2F)C(=O)C1=O] | 618.32 | 619.2 |
| 52 | 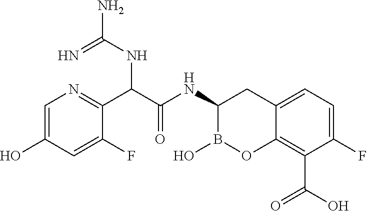 [SMILES: N=C(N)NC(C(=O)N[C@H]1Cc2ccc(F)c(C(=O)O)c2OB1O)c1ncc(O)cc1F] | 435.15 | 436.1 |
| 53 | 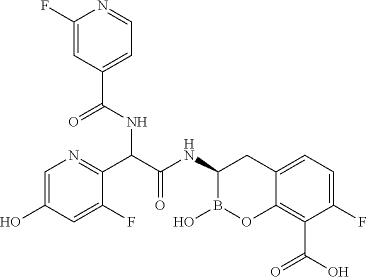 [SMILES: O=C(NC(C(=O)N[C@H]1Cc2ccc(F)c(C(=O)O)c2OB1O)c1ncc(O)cc1F)c1ccnc(F)c1] | 516.20 | 516.6 |
| 54 | 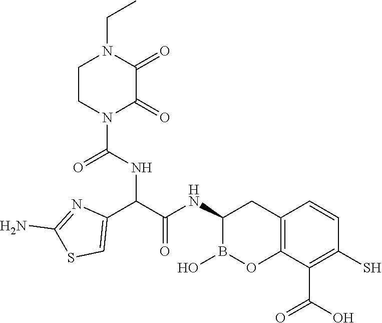 [SMILES: CCN1CCN(C(=O)NC(C(=O)N[C@H]2Cc3ccc(S)c(C(=O)O)c3OB2O)c2csc(N)n2)C(=O)C1=O] | 562.38 | 562.2 |
| 55 | 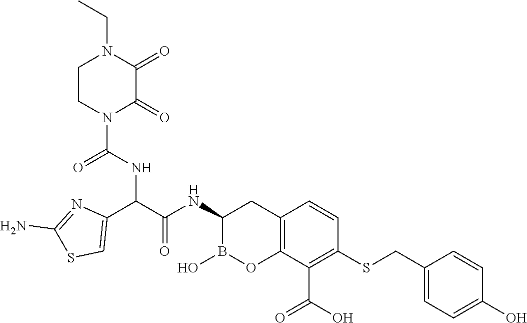 [SMILES: CCN1CCN(C(=O)NC(C(=O)N[C@H]2Cc3ccc(SCc4ccc(O)cc4)c(C(=O)O)c3OB2O)c2csc(N)n2)C(=O)C1=O] | 668.50 | 690.9 [M + Na]⁺ |
| 56 | 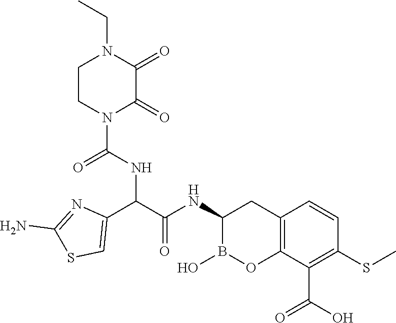 [SMILES: CCN1CCN(C(=O)NC(C(=O)N[C@H]2Cc3ccc(SC)c(C(=O)O)c3OB2O)c2csc(N)n2)C(=O)C1=O] | 576.41 | 577.1 |
| 57 | 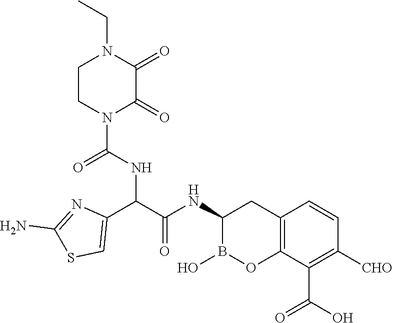 [SMILES: CCN1CCN(C(=O)NC(C(=O)N[C@H]2Cc3ccc(C=O)c(C(=O)O)c3OB2O)c2csc(N)n2)C(=O)C1=O] | 558.33 | 559 |
| 58 | 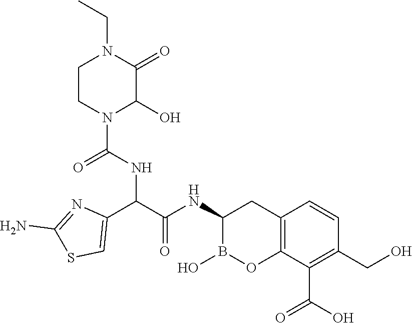 [SMILES: CCN1CCN(C(=O)NC(C(=O)N[C@H]2Cc3ccc(CO)c(C(=O)O)c3OB2O)c2csc(N)n2)C(O)C1=O] | 562.36 | 563 |
| 59 | 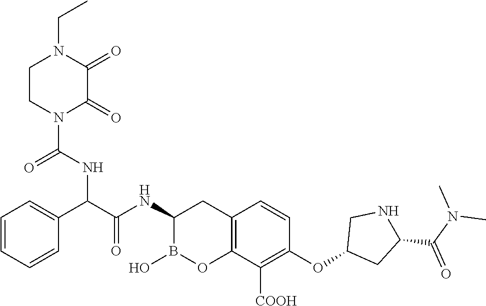 [SMILES: CCN1CCN(C(=O)NC(C(=O)N[C@H]2Cc3ccc(O[C@@H]4CN[C@H](C(=O)N(C)C)C4)c(C(=O)O)c3OB2O)c2ccccc2)C(=O)C1=O] | 664.48 | 665.3 |
| 60 | 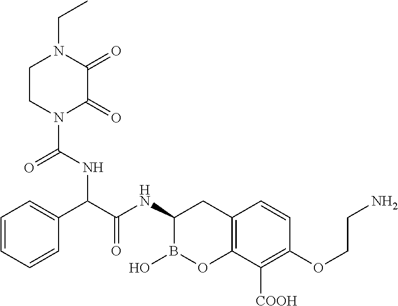 [SMILES: CCN1CCN(C(=O)NC(C(=O)N[C@H]2Cc3ccc(OCCN)c(C(=O)O)c3OB2O)c2ccccc2)C(=O)C1=O] | 567.36 | 568.2 |
| 61 | 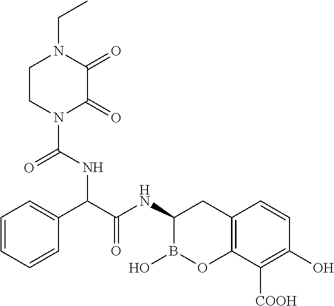 [SMILES: CCN1CCN(C(=O)NC(C(=O)N[C@H]2Cc3ccc(O)c(C(=O)O)c3OB2O)c2ccccc2)C(=O)C1=O] | 524.29 | 525.3 |
| 62 | 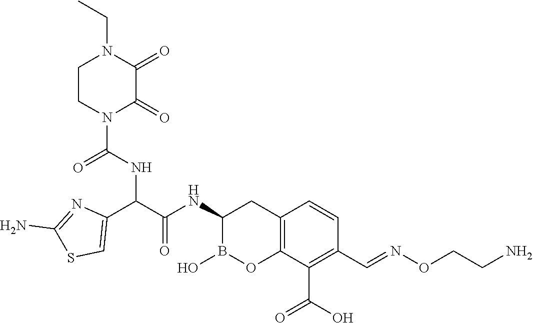 [SMILES: CCN1CCN(C(=O)NC(C(=O)N[C@H]2Cc3ccc(/C=N/OCCN)c(C(=O)O)c3OB2O)c2csc(N)n2)C(=O)C1=O] | 616.41 | 617 |
| 63 | 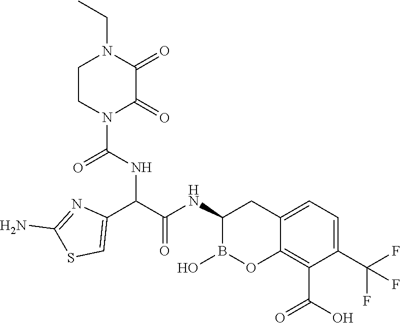 [SMILES: CCN1CCN(C(=O)NC(C(=O)N[C@H]2Cc3ccc(C(F)(F)F)c(C(=O)O)c3OB2O)c2csc(N)n2)C(=O)C1=O] | 598.32 | 599.1 |
| 64 | 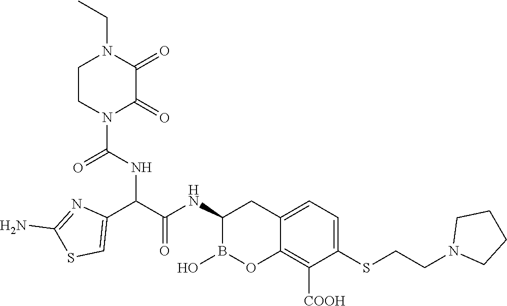 [SMILES: CCN1CCN(C(=O)NC(C(=O)N[C@H]2Cc3ccc(SCCN4CCCC4)c(C(=O)O)c3OB2O)c2csc(N)n2)C(=O)C1=O] | 659.54 | 660.2 |
| 65 | 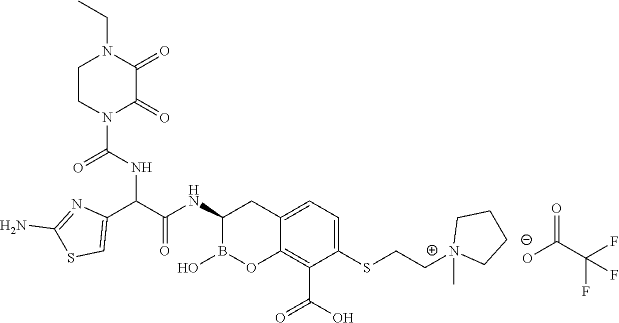 | 673.57 | 674.2 (M)⁺ |
| 66 | 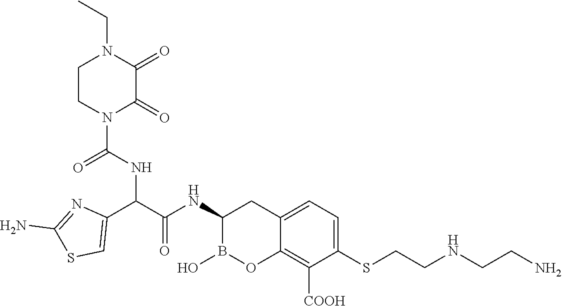 [SMILES: CCN1CCN(C(=O)NC(C(=O)N[C@H]2Cc3ccc(SCCNCCN)c(C(=O)O)c3OB2O)c2csc(N)n2)C(=O)C1=O] | 648.52 | 649.2 |
| 67 | 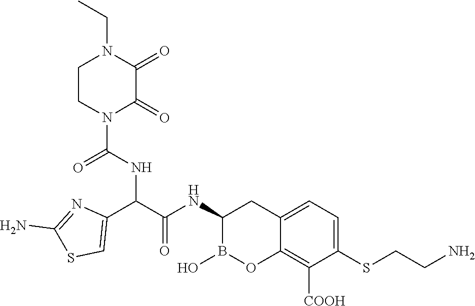 [SMILES: CCN1CCN(C(=O)NC(C(=O)N[C@H]2Cc3ccc(SCCN)c(C(=O)O)c3OB2O)c2csc(N)n2)C(=O)C1=O] | 605.45 | 606.1 |
| 68 | 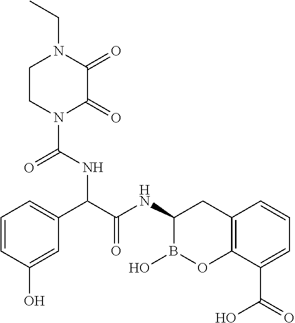 [SMILES: CCN1CCN(C(=O)NC(C(=O)N[C@H]2Cc3cccc(C(=O)O)c3OB2O)c2cccc(O)c2)C(=O)C1=O] | 524.3 | 525 |
| 69 | 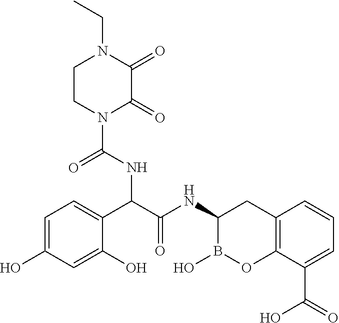 [SMILES: CCN1CCN(C(=O)NC(C(=O)N[C@H]2Cc3cccc(C(=O)O)c3OB2O)c2ccc(O)cc2O)C(=O)C1=O] | 540.3 | 541 |
| 70 | 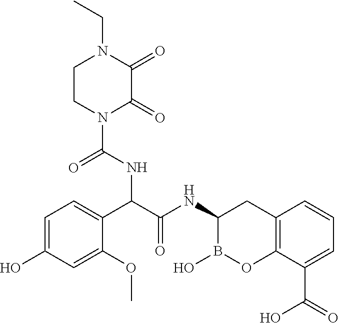 [SMILES: CCN1CCN(C(=O)NC(C(=O)N[C@H]2Cc3cccc(C(=O)O)c3OB2O)c2ccc(O)cc2OC)C(=O)C1=O] | 554.3 | 555 |
| 71 | 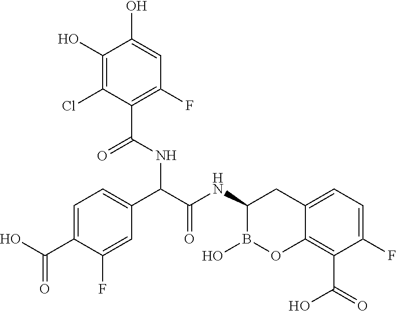 [SMILES: O=C(O)c1ccc(C(NC(=O)c2c(F)cc(O)c(O)c2Cl)C(=O)N[C@H]2Cc3ccc(F)c(C(=O)O)c3OB2O)cc1F] | 608.7 | 609.0 |
| 72 | 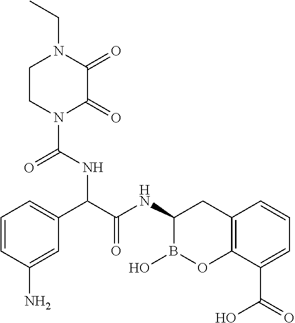 [SMILES: CCN1CCN(C(=O)NC(C(=O)N[C@H]2Cc3cccc(C(=O)O)c3OB2O)c2cccc(N)c2)C(=O)C1=O] | 523.3 | 524 |
| 73 | 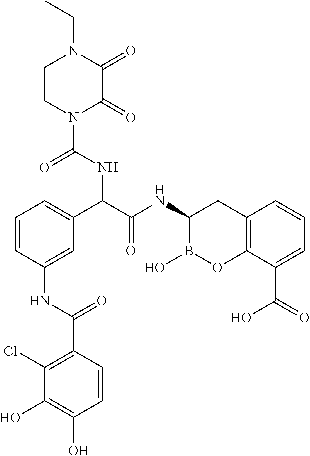 [SMILES: CCN1CCN(C(=O)NC(C(=O)N[C@H]2Cc3cccc(C(=O)O)c3OB2O)c2cccc(NC(=O)c3ccc(O)c(O)c3Cl)c2)C(=O)C1=O] | 693.9 | 694 |
| 74 | 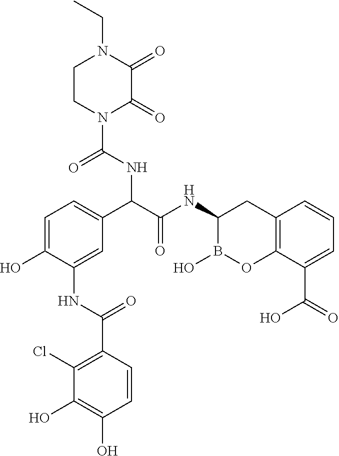 [SMILES: CCN1CCN(C(=O)NC(C(=O)N[C@H]2Cc3cccc(C(=O)O)c3OB2O)c2ccc(O)c(NC(=O)c3ccc(O)c(O)c3Cl)c2)C(=O)C1=O] | 709.9 | 710 |
| 75 | 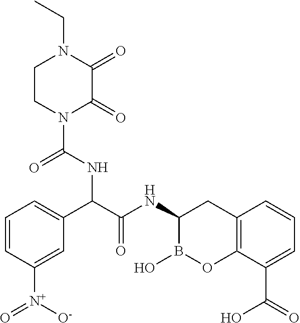 [SMILES: CCN1CCN(C(=O)NC(C(=O)N[C@H]2Cc3cccc(C(=O)O)c3OB2O)c2cccc([N+](=O)[O-])c2)C(=O)C1=O] | 553.3 | 554 |
| 76 | 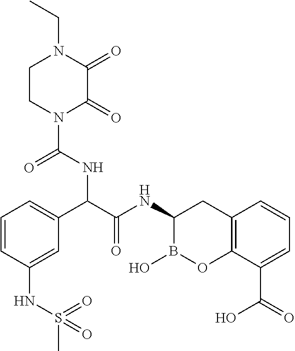 [SMILES: CCN1CCN(C(=O)NC(C(=O)N[C@H]2Cc3cccc(C(=O)O)c3OB2O)c2cccc(NS(C)(=O)=O)c2)C(=O)C1=O] | 601.4 | 602 |
| 77 | 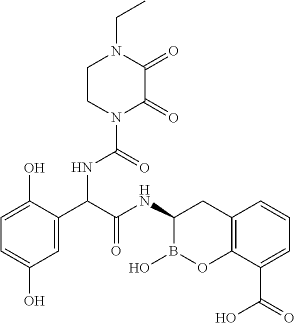 [SMILES: CCN1CCN(C(=O)NC(C(=O)N[C@H]2Cc3cccc(C(=O)O)c3OB2O)c2cc(O)ccc2O)C(=O)C1=O] | 540.3 | 541 |
| 78 | 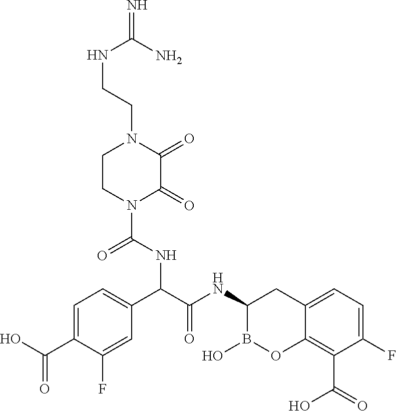 [SMILES: N=C(N)NCCN1CCN(C(=O)NC(C(=O)N[C@H]2Cc3ccc(F)c(C(=O)O)c3OB2O)c2ccc(C(=O)O)c(F)c2)C(=O)C1=O] | 645.3 | 646.2 |
| 79 | 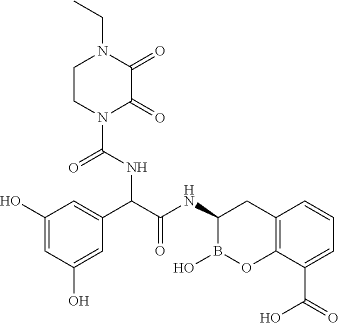 [SMILES: CCN1CCN(C(=O)NC(C(=O)N[C@H]2Cc3cccc(C(=O)O)c3OB2O)c2cc(O)cc(O)c2)C(=O)C1=O] | 540.3 | 541 |
| 80 | 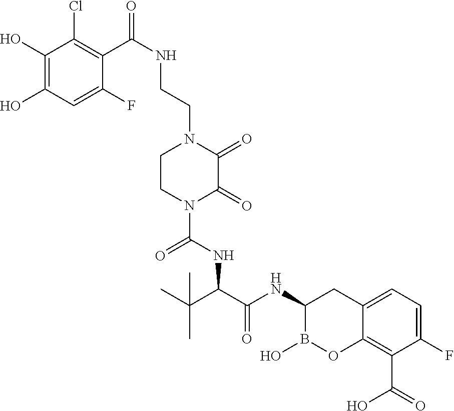 [SMILES: CC(C)(C)[C@@H](NC(=O)N1CCN(CCNC(=O)c2c(F)cc(O)c(O)c2Cl)C(=O)C1=O)C(=O)N[C@H]1Cc2ccc(F)c(C(=O)O)c2OB1O] | 709.8 | 710.2 |
| 81 | 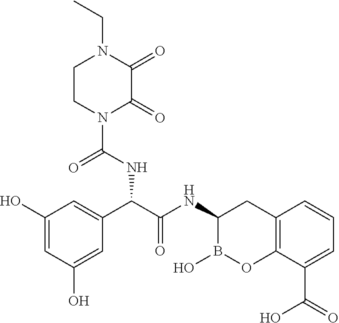 [SMILES: CCN1CCN(C(=O)N[C@H](C(=O)N[C@H]2Cc3cccc(C(=O)O)c3OB2O)c2cc(O)cc(O)c2)C(=O)C1=O] | 540.3 | 541 |
| 82 | 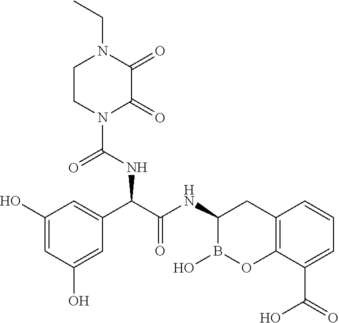 [SMILES: CCN1CCN(C(=O)N[C@@H](C(=O)N[C@H]2Cc3cccc(C(=O)O)c3OB2O)c2cc(O)cc(O)c2)C(=O)C1=O] | 540.3 | 541 |
| 83 | 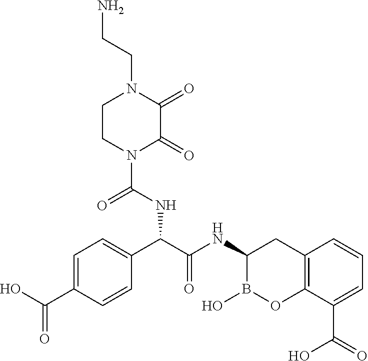 [SMILES: NCCN1CCN(C(=O)N[C@H](C(=O)N[C@H]2Cc3cccc(C(=O)O)c3OB2O)c2ccc(C(=O)O)cc2)C(=O)C1=O] | 567.3 | 568.2 |
| 84 | 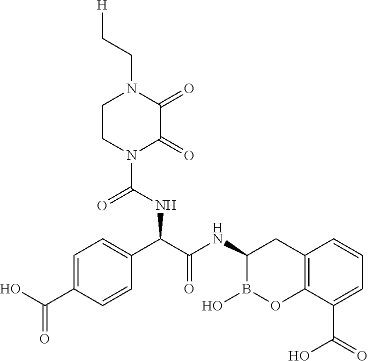 [SMILES: CCN1CCN(C(=O)N[C@@H](C(=O)N[C@H]2Cc3cccc(C(=O)O)c3OB2O)c2ccc(C(=O)O)cc2)C(=O)C1=O] | 567.3 | 568.3 |
| 85 | 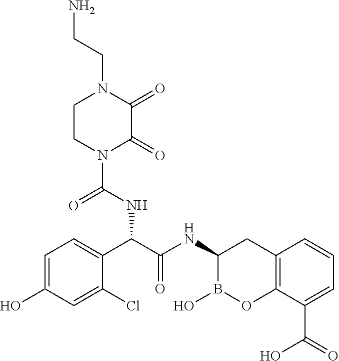 [SMILES: NCCN1CCN(C(=O)N[C@H](C(=O)N[C@H]2Cc3cccc(C(=O)O)c3OB2O)c2ccc(O)cc2Cl)C(=O)C1=O] | 573.8 | 574.0 |
| 86 | 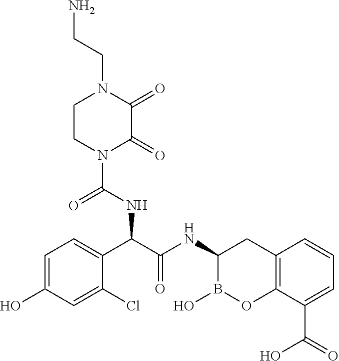 [SMILES: NCCN1CCN(C(=O)N[C@@H](C(=O)N[C@H]2Cc3cccc(C(=O)O)c3OB2O)c2ccc(O)cc2Cl)C(=O)C1=O] | 573.8 | 574.0 |
| 87 | 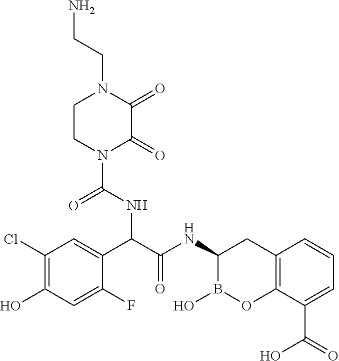 [SMILES: NCCN1CCN(C(=O)NC(C(=O)N[C@H]2Cc3cccc(C(=O)O)c3OB2O)c2cc(Cl)c(O)cc2F)C(=O)C1=O] | 591.7 | 592.0 |
| 88 | 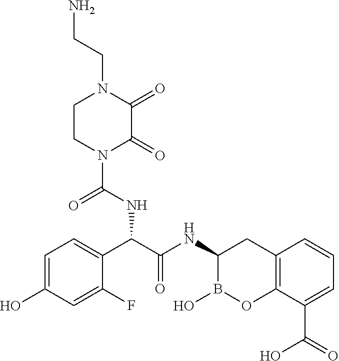 [SMILES: NCCN1CCN(C(=O)N[C@H](C(=O)N[C@H]2Cc3cccc(C(=O)O)c3OB2O)c2ccc(O)cc2F)C(=O)C1=O] | 557.3 | 558.0 |
| 89 | 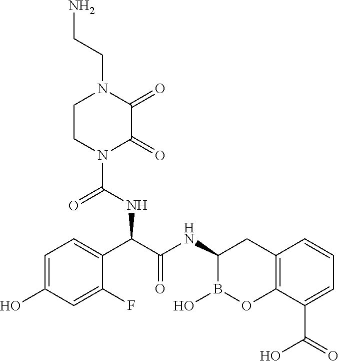 [SMILES: NCCN1CCN(C(=O)N[C@@H](C(=O)N[C@H]2Cc3cccc(C(=O)O)c3OB2O)c2ccc(O)cc2F)C(=O)C1=O] | 557.3 | 558.1 |
| 90 | 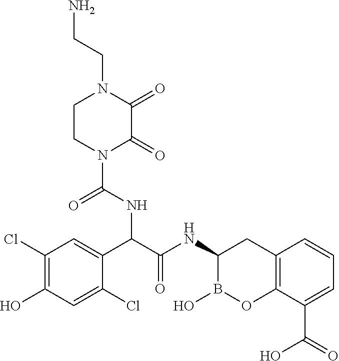 [SMILES: NCCN1CCN(C(=O)NC(C(=O)N[C@H]2Cc3cccc(C(=O)O)c3OB2O)c2cc(Cl)c(O)cc2Cl)C(=O)C1=O] | 608.2 | 608.0 |
| 91 | 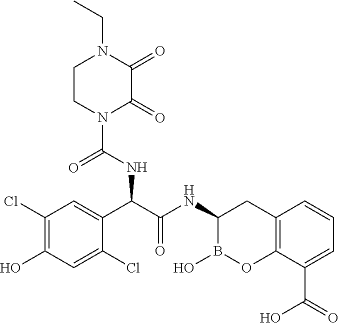 [SMILES: CCN1CCN(C(=O)N[C@@H](C(=O)N[C@H]2Cc3cccc(C(=O)O)c3OB2O)c2cc(Cl)c(O)cc2Cl)C(=O)C1=O] | 593.2 | 592.5 |
| 92 | 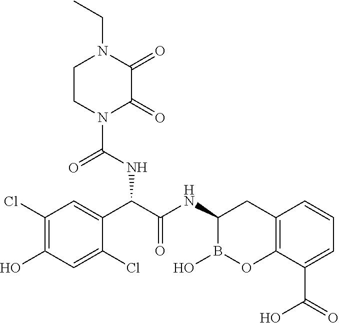 [SMILES: CCN1CCN(C(=O)N[C@H](C(=O)N[C@H]2Cc3cccc(C(=O)O)c3OB2O)c2cc(Cl)c(O)cc2Cl)C(=O)C1=O] | 593.2 | 592.5 |
| 93 | 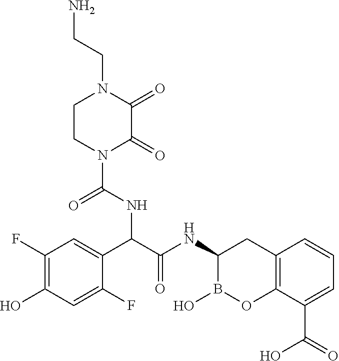 [SMILES: NCCN1CCN(C(=O)NC(C(=O)N[C@H]2Cc3cccc(C(=O)O)c3OB2O)c2cc(F)c(O)cc2F)C(=O)C1=O] | 575.3 | 575.5 |
| 94 | 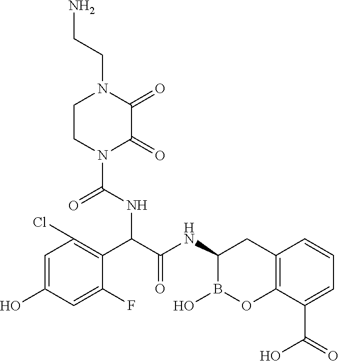 [SMILES: NCCN1CCN(C(=O)NC(C(=O)N[C@H]2Cc3cccc(C(=O)O)c3OB2O)c2c(F)cc(O)cc2Cl)C(=O)C1=O] | 591.7 | 592.0 |
| 95 | 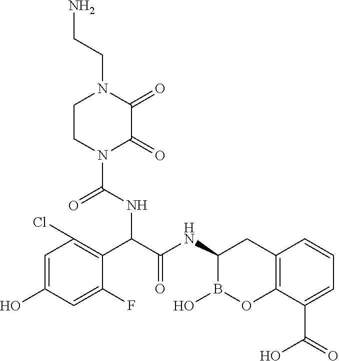 [SMILES: NCCN1CCN(C(=O)NC(C(=O)N[C@H]2Cc3cccc(C(=O)O)c3OB2O)c2c(F)cc(O)cc2Cl)C(=O)C1=O] | 576.7 | 577.0 |
| 96 | 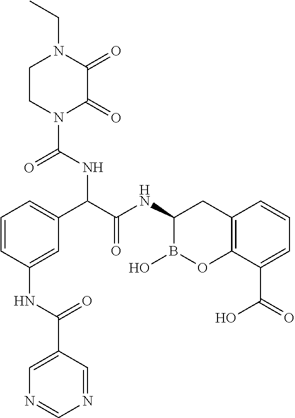 [SMILES: CCN1CCN(C(=O)NC(C(=O)N[C@H]2Cc3cccc(C(=O)O)c3OB2O)c2cccc(NC(=O)c3cncnc3)c2)C(=O)C1=O] | 629.4 | 630 |
| 97 | 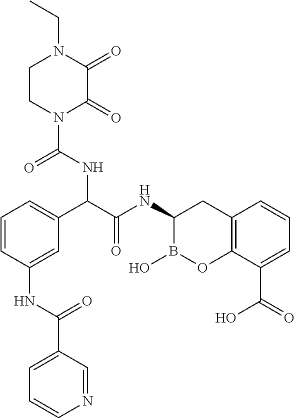 [SMILES: CCN1CCN(C(=O)NC(C(=O)N[C@H]2Cc3cccc(C(=O)O)c3OB2O)c2cccc(NC(=O)c3cccnc3)c2)C(=O)C1=O] | 628.4 | 629 |
| 98 | 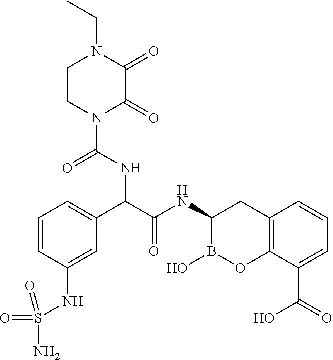 [SMILES: CCN1CCN(C(=O)NC(C(=O)N[C@H]2Cc3cccc(C(=O)O)c3OB2O)c2cccc(NS(N)(=O)=O)c2)C(=O)C1=O] | 602.4 | 603 |
| 99 | 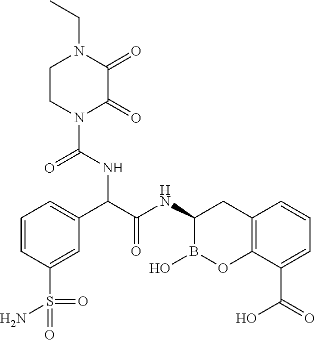 [SMILES: CCN1CCN(C(=O)NC(C(=O)N[C@H]2Cc3cccc(C(=O)O)c3OB2O)c2cccc(S(N)(=O)=O)c2)C(=O)C1=O] | 587.4 | 588 |
| 100 | 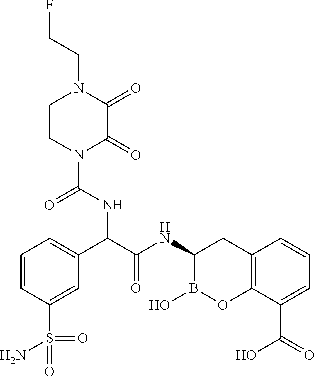 [SMILES: NS(=O)(=O)c1cccc(C(NC(=O)N2CCN(CCF)C(=O)C2=O)C(=O)N[C@H]2Cc3cccc(C(=O)O)c3OB2O)c1] | 605.4 | 606 |
| 101 | 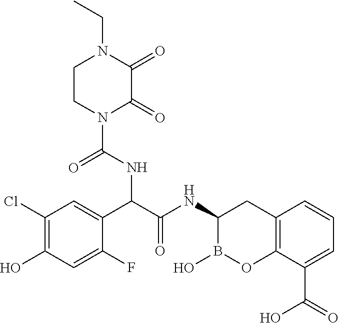 [SMILES: CCN1CCN(C(=O)NC(C(=O)N[C@H]2Cc3cccc(C(=O)O)c3OB2O)c2cc(Cl)c(O)cc2F)C(=O)C1=O] | 576.7 | 577.2 |
| 102 | 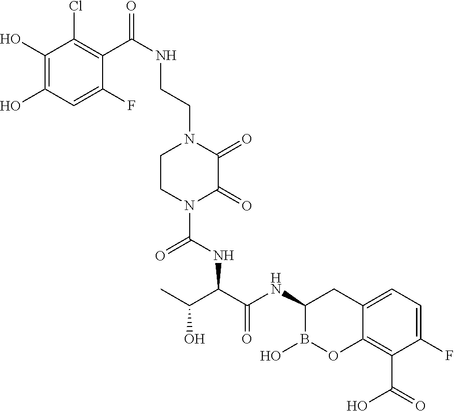 [SMILES: C[C@@H](O)[C@@H](NC(=O)N1CCN(CCNC(=O)c2c(F)cc(O)c(O)c2Cl)C(=O)C1=O)C(=O)N[C@H]1Cc2ccc(F)c(C(=O)O)c2OB1O] | 697.8 | 698.2 |
| 103 | 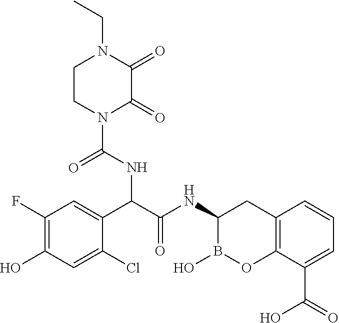 [SMILES: CCN1CCN(C(=O)NC(C(=O)N[C@H]2Cc3cccc(C(=O)O)c3OB2O)c2cc(F)c(O)cc2Cl)C(=O)C1=O] | 576.7 | 576.6 |
| 104 | 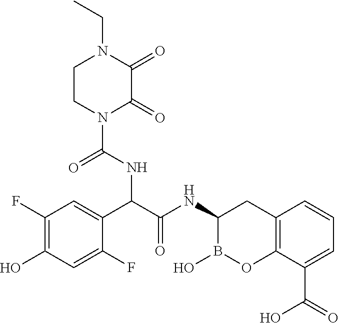 [SMILES: CCN1CCN(C(=O)NC(C(=O)N[C@H]2Cc3cccc(C(=O)O)c3OB2O)c2cc(F)c(O)cc2F)C(=O)C1=O] | 560.3 | 560.6 |
| 105 | 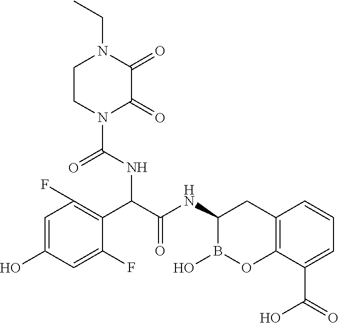 [SMILES: CCN1CCN(C(=O)NC(C(=O)N[C@H]2Cc3cccc(C(=O)O)c3OB2O)c2c(F)cc(O)cc2F)C(=O)C1=O] | 560.3 | 560.6 |
| 106 | 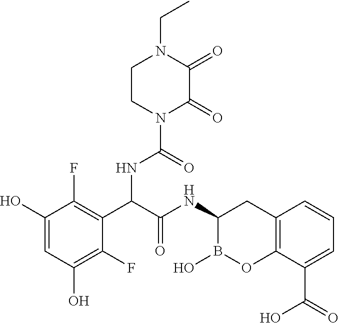 [SMILES: CCN1CCN(C(=O)NC(C(=O)N[C@H]2Cc3cccc(C(=O)O)c3OB2O)c2c(F)c(O)cc(O)c2F)C(=O)C1=O] | 576.3 | 577 |
| 107 | 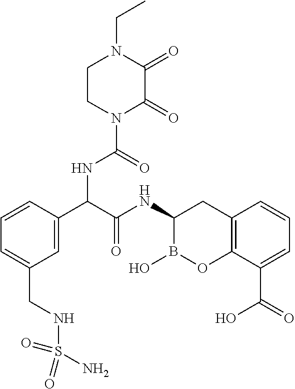 [SMILES: CCN1CCN(C(=O)NC(C(=O)N[C@H]2Cc3cccc(C(=O)O)c3OB2O)c2cccc(CNS(N)(=O)=O)c2)C(=O)C1=O] | 616.4 | 617 |
| 108 | 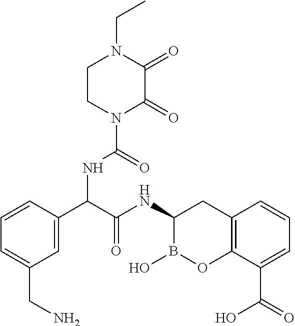 [SMILES: CCN1CCN(C(=O)NC(C(=O)N[C@H]2Cc3cccc(C(=O)O)c3OB2O)c2cccc(CN)c2)C(=O)C1=O] | 537.3 | 538 |
| 109 | 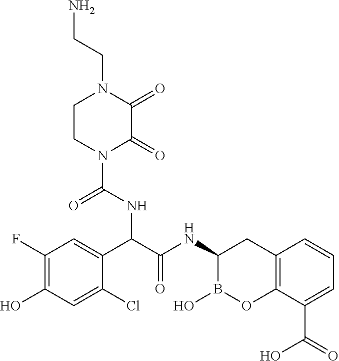 [SMILES: NCCN1CCN(C(=O)NC(C(=O)N[C@H]2Cc3cccc(C(=O)O)c3OB2O)c2cc(F)c(O)cc2Cl)C(=O)C1=O] | 591.7 | 592.0 |
| 110 | 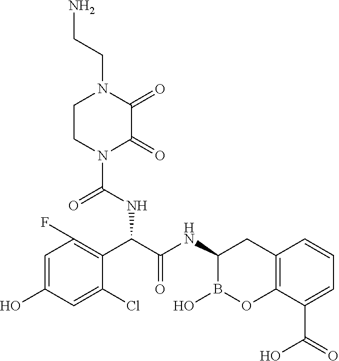 [SMILES: NCCN1CCN(C(=O)N[C@H](C(=O)N[C@H]2Cc3cccc(C(=O)O)c3OB2O)c2c(F)cc(O)cc2Cl)C(=O)C1=O] | 575.3 | 576.0 |
| 111 | 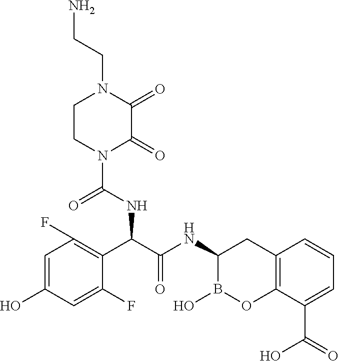 [SMILES: NCCN1CCN(C(=O)N[C@@H](C(=O)N[C@H]2Cc3cccc(C(=O)O)c3OB2O)c2c(F)cc(O)cc2F)C(=O)C1=O] | 575.3 | 576.1 |
| 112 | 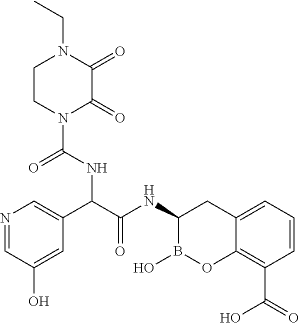 [SMILES: CCN1CCN(C(=O)NC(C(=O)N[C@H]2Cc3cccc(C(=O)O)c3OB2O)c2cncc(O)c2)C(=O)C1=O] | 525.3 | 526.2 |
| 113 | 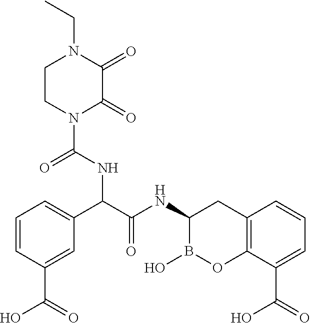 [SMILES: CCN1CCN(C(=O)NC(C(=O)N[C@H]2Cc3cccc(C(=O)O)c3OB2O)c2cccc(C(=O)O)c2)C(=O)C1=O] | 552.3 | 553 |
| 114 | 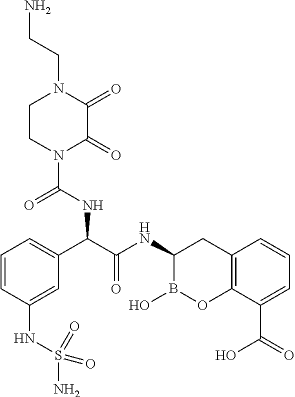 [SMILES: NCCN1CCN(C(=O)N[C@@H](C(=O)N[C@H]2Cc3cccc(C(=O)O)c3OB2O)c2cccc(NS(N)(=O)=O)c2)C(=O)C1=O] | 617.4 | 618 |
| 115 | 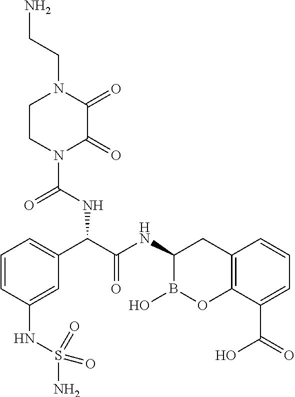 [SMILES: NCCN1CCN(C(=O)N[C@H](C(=O)N[C@H]2Cc3cccc(C(=O)O)c3OB2O)c2cccc(NS(N)(=O)=O)c2)C(=O)C1=O] | 617.4 | 618 |
| 116 | 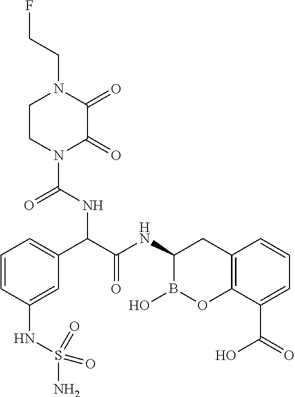 [SMILES: NS(=O)(=O)Nc1cccc(C(NC(=O)N2CCN(CCF)C(=O)C2=O)C(=O)N[C@H]2Cc3cccc(C(=O)O)c3OB2O)c1] | 620.4 | 621 |
| 117 | 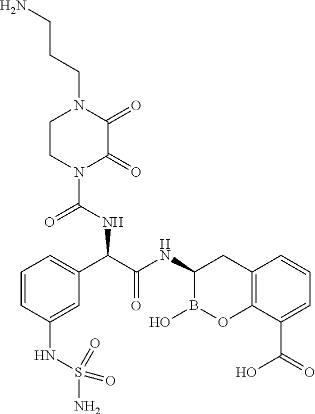 [SMILES: NCCCN1CCN(C(=O)N[C@@H](C(=O)N[C@H]2Cc3cccc(C(=O)O)c3OB2O)c2cccc(NS(N)(=O)=O)c2)C(=O)C1=O] | 631.4 | 632 |
| 118 | 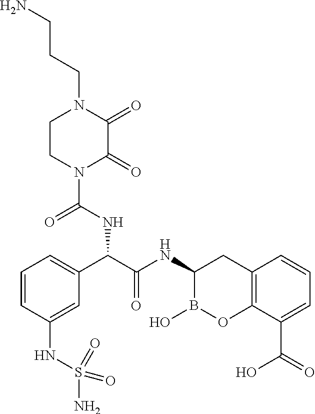 [SMILES: NCCCN1CCN(C(=O)N[C@H](C(=O)N[C@H]2Cc3cccc(C(=O)O)c3OB2O)c2cccc(NS(N)(=O)=O)c2)C(=O)C1=O] | 631.4 | 632 |
| 119 | 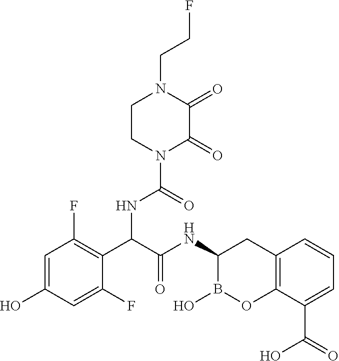 [SMILES: O=C(O)c1cccc2c1OB(O)[C@@H](NC(=O)C(NC(=O)N1CCN(CCF)C(=O)C1=O)c1c(F)cc(O)cc1F)C2] | 578.3 | 579 |
| 120 | 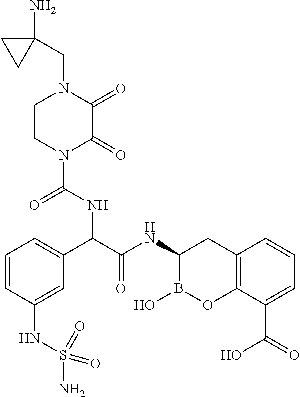 [SMILES: NC1(CN2CCN(C(=O)NC(C(=O)N[C@H]3Cc4cccc(C(=O)O)c4OB3O)c3cccc(NS(N)(=O)=O)c3)C(=O)C2=O)CC1] | 643.4 | 644 |
| 121 | 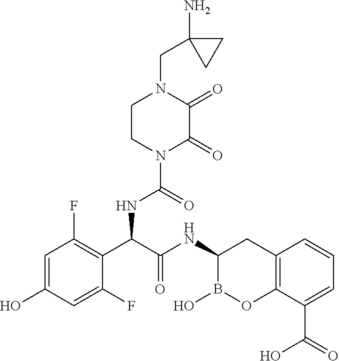 [SMILES: NC1(CN2CCN(C(=O)N[C@@H](C(=O)N[C@H]3Cc4cccc(C(=O)O)c4OB3O)c3c(F)cc(O)cc3F)C(=O)C2=O)CC1] | 601.3 | 602 |
| 122 | 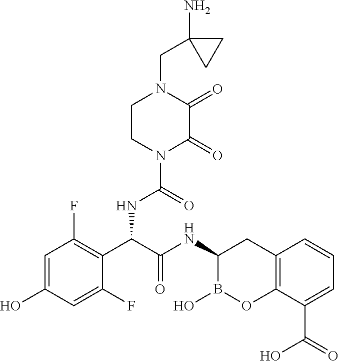 [SMILES: NC1(CN2CCN(C(=O)N[C@H](C(=O)N[C@H]3Cc4cccc(C(=O)O)c4OB3O)c3c(F)cc(O)cc3F)C(=O)C2=O)CC1] | 601.3 | 602 |
| 123 | 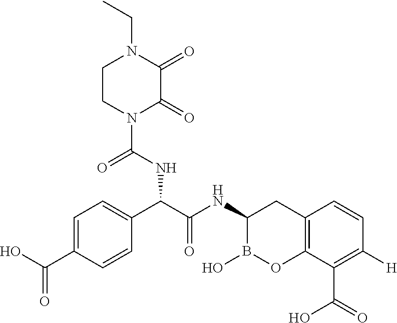 [SMILES: CCN1CCN(C(=O)N[C@H](C(=O)N[C@H]2Cc3cccc(C(=O)O)c3OB2O)c2ccc(C(=O)O)cc2)C(=O)C1=O] | 552.3 | 553.2 |
| 124 | 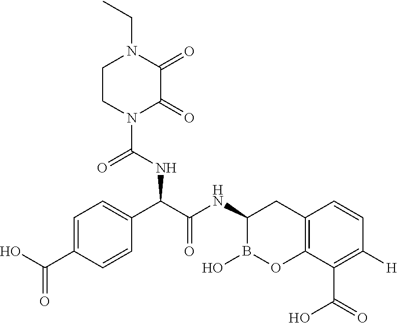 [SMILES: CCN1CCN(C(=O)N[C@@H](C(=O)N[C@H]2Cc3cccc(C(=O)O)c3OB2O)c2ccc(C(=O)O)cc2)C(=O)C1=O] | 552.3 | 553.2 |
| 125 | 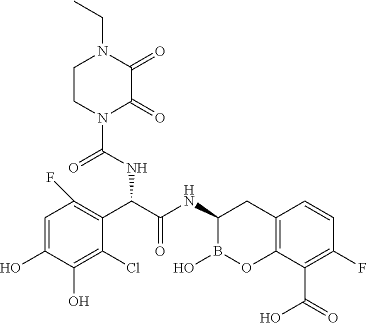 [SMILES: CCN1CCN(C(=O)N[C@H](C(=O)N[C@H]2Cc3ccc(F)c(C(=O)O)c3OB2O)c2c(F)cc(O)c(O)c2Cl)C(=O)C1=O] | 610.7 | 611.1 |
| 126 | 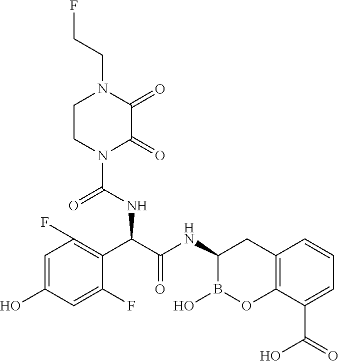 [SMILES: O=C(O)c1cccc2c1OB(O)[C@@H](NC(=O)[C@H](NC(=O)N1CCN(CCF)C(=O)C1=O)c1c(F)cc(O)cc1F)C2] | 578.3 | 579 |
| 127 | 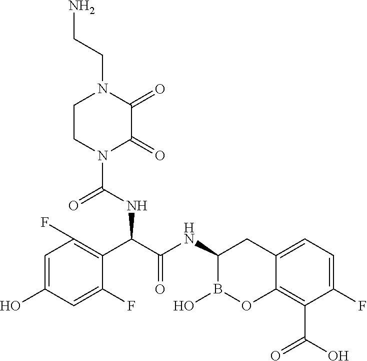 [SMILES: NCCN1CCN(C(=O)N[C@@H](C(=O)N[C@H]2Cc3ccc(F)c(C(=O)O)c3OB2O)c2c(F)cc(O)cc2F)C(=O)C1=O] | 593.3 | 594.2 |
| 128 | 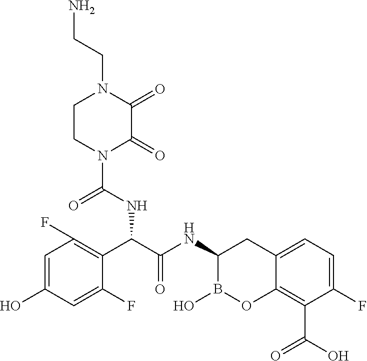 [SMILES: NCCN1CCN(C(=O)N[C@H](C(=O)N[C@H]2Cc3ccc(F)c(C(=O)O)c3OB2O)c2c(F)cc(O)cc2F)C(=O)C1=O] | 593.3 | 594.2 |
| 129 | 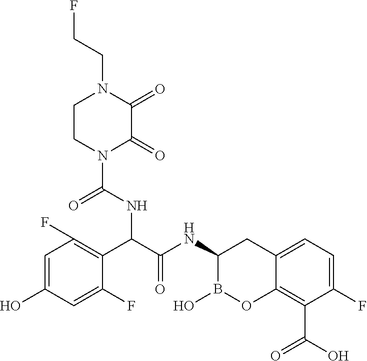 [SMILES: O=C(O)c1c(F)ccc2c1OB(O)[C@@H](NC(=O)C(NC(=O)N1CCN(CCF)C(=O)C1=O)c1c(F)cc(O)cc1F)C2] | 596.3 | 597.2 |
| 130 | 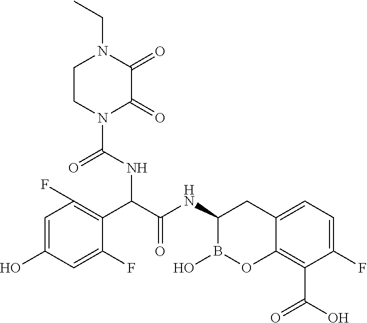 [SMILES: CCN1CCN(C(=O)NC(C(=O)N[C@H]2Cc3ccc(F)c(C(=O)O)c3OB2O)c2c(F)cc(O)cc2F)C(=O)C1=O] | 578.3 | 579.2 |
| 131 | 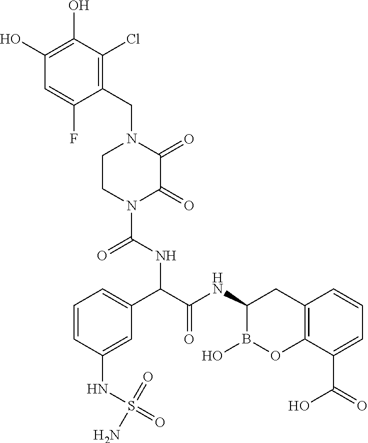 [SMILES: NS(=O)(=O)Nc1cccc(C(NC(=O)N2CCN(Cc3c(F)cc(O)c(O)c3Cl)C(=O)C2=O)C(=O)N[C@H]2Cc3cccc(C(=O)O)c3OB2O)c1] | 748.9 | 749 |
| 132 | 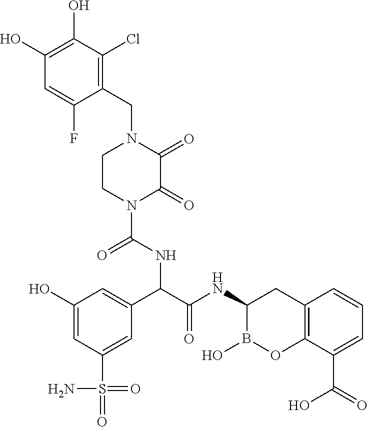 [SMILES: NS(=O)(=O)c1cc(O)cc(C(NC(=O)N2CCN(Cc3c(F)cc(O)c(O)c3Cl)C(=O)C2=O)C(=O)N[C@H]2Cc3cccc(C(=O)O)c3OB2O)c1] | 749.9 | 750 |
| 133 | 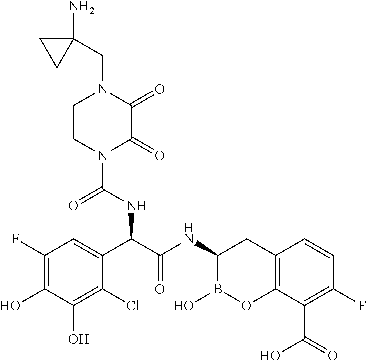 [SMILES: NC1(CN2CCN(C(=O)N[C@@H](C(=O)N[C@H]3Cc4ccc(F)c(C(=O)O)c4OB3O)c3cc(F)c(O)c(O)c3Cl)C(=O)C2=O)CC1] | 651.8 | 652 |
| 134 | 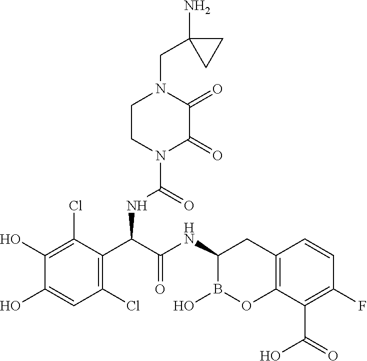 [SMILES: NC1(CN2CCN(C(=O)N[C@@H](C(=O)N[C@H]3Cc4ccc(F)c(C(=O)O)c4OB3O)c3c(Cl)cc(O)c(O)c3Cl)C(=O)C2=O)CC1] | 651.8 | 652 |
| 135 | 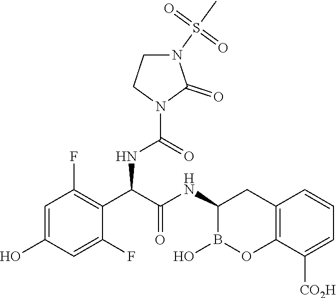 [SMILES: CS(=O)(=O)N1CCN(C(=O)N[C@@H](C(=O)N[C@H]2Cc3cccc(C(=O)O)c3OB2O)c2c(F)cc(O)cc2F)C1=O] | 582.3 | 583.0 |
| 136 | 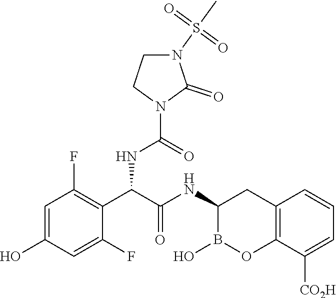 [SMILES: CS(=O)(=O)N1CCN(C(=O)N[C@H](C(=O)N[C@H]2Cc3cccc(C(=O)O)c3OB2O)c2c(F)cc(O)cc2F)C1=O] | 582.3 | 583.0 |
| 137 | 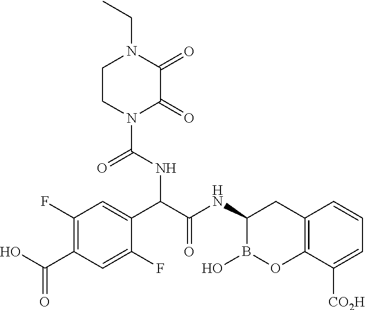 [SMILES: CCN1CCN(C(=O)NC(C(=O)N[C@H]2Cc3cccc(C(=O)O)c3OB2O)c2cc(F)c(C(=O)O)cc2F)C(=O)C1=O] | 588.3 | 589.1 |
| 138 | 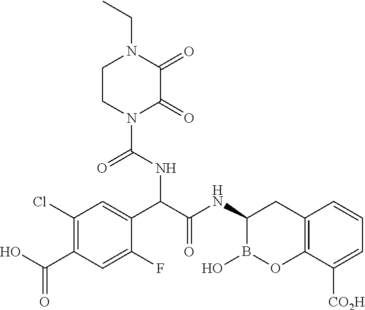 [SMILES: CCN1CCN(C(=O)NC(C(=O)N[C@H]2Cc3cccc(C(=O)O)c3OB2O)c2cc(Cl)c(C(=O)O)cc2F)C(=O)C1=O] | 604.7 | 605.0 |
| 139 | 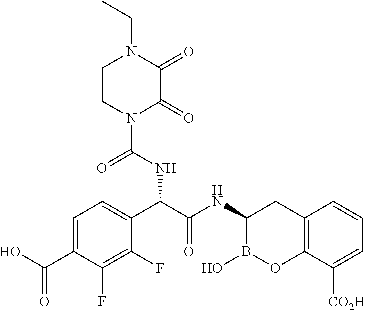 [SMILES: CCN1CCN(C(=O)N[C@H](C(=O)N[C@H]2Cc3cccc(C(=O)O)c3OB2O)c2ccc(C(=O)O)c(F)c2F)C(=O)C1=O] | 588.3 | 589.2 |
| 140 | 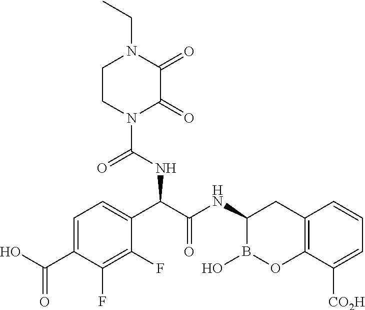 [SMILES: CCN1CCN(C(=O)N[C@@H](C(=O)N[C@H]2Cc3cccc(C(=O)O)c3OB2O)c2ccc(C(=O)O)c(F)c2F)C(=O)C1=O] | 588.3 | 589.2 |
| 141 | 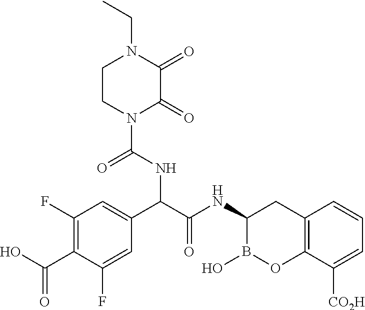 [SMILES: CCN1CCN(C(=O)NC(C(=O)N[C@H]2Cc3cccc(C(=O)O)c3OB2O)c2cc(F)c(C(=O)O)c(F)c2)C(=O)C1=O] | 588.3 | 589.0 |
| 142 | 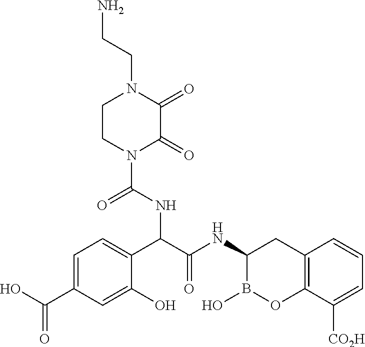 [SMILES: NCCN1CCN(C(=O)NC(C(=O)N[C@H]2Cc3cccc(C(=O)O)c3OB2O)c2ccc(C(=O)O)cc2O)C(=O)C1=O] | 583.3 | 584.0 |
| 143 | 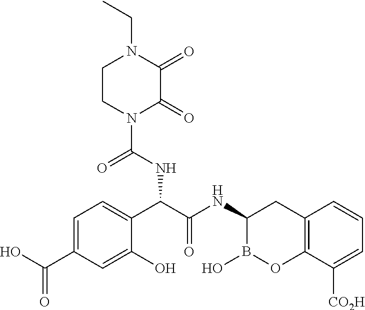 [SMILES: CCN1CCN(C(=O)N[C@H](C(=O)N[C@H]2Cc3cccc(C(=O)O)c3OB2O)c2ccc(C(=O)O)cc2O)C(=O)C1=O] | 568.3 | 569.1 |
| 144 | 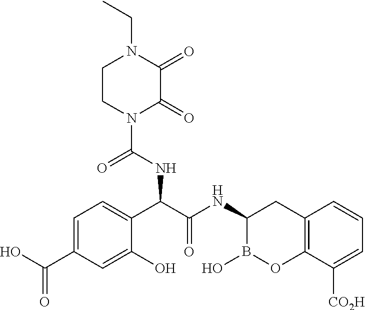 [SMILES: CCN1CCN(C(=O)N[C@@H](C(=O)N[C@H]2Cc3cccc(C(=O)O)c3OB2O)c2ccc(C(=O)O)cc2O)C(=O)C1=O] | 568.3 | 569.0 |
| 145 | 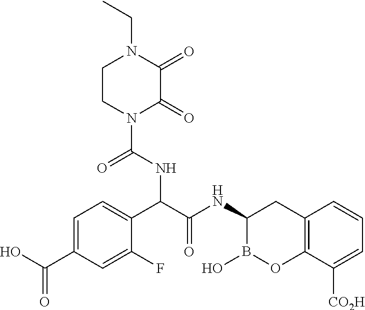 [SMILES: CCN1CCN(C(=O)NC(C(=O)N[C@H]2Cc3cccc(C(=O)O)c3OB2O)c2ccc(C(=O)O)cc2F)C(=O)C1=O] | 570.3 | 571.0 |
| 146 | 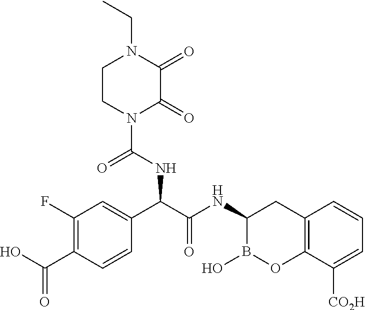 [SMILES: CCN1CCN(C(=O)N[C@@H](C(=O)N[C@H]2Cc3cccc(C(=O)O)c3OB2O)c2ccc(C(=O)O)c(F)c2)C(=O)C1=O] | 570.3 | 570.7 |
| 147 | 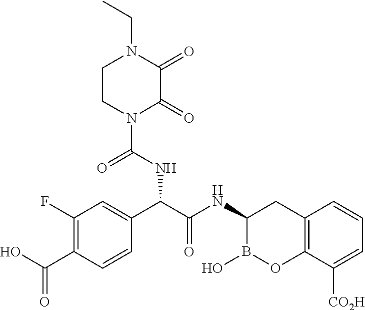 [SMILES: CCN1CCN(C(=O)N[C@H](C(=O)N[C@H]2Cc3cccc(C(=O)O)c3OB2O)c2ccc(C(=O)O)c(F)c2)C(=O)C1=O] | 570.3 | 570.6 |
| 148 | 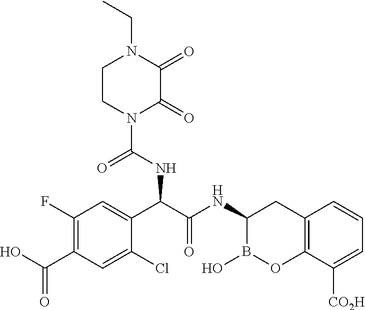 [SMILES: CCN1CCN(C(=O)N[C@@H](C(=O)N[C@H]2Cc3cccc(C(=O)O)c3OB2O)c2cc(F)c(C(=O)O)cc2Cl)C(=O)C1=O] | 604.7 | 604.6 |
| 149 | 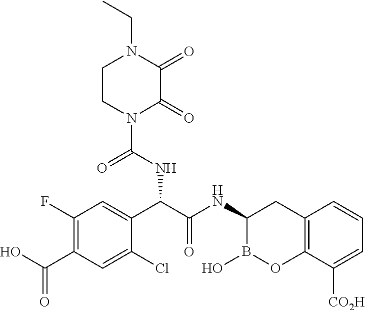 [SMILES: CCN1CCN(C(=O)N[C@H](C(=O)N[C@H]2Cc3cccc(C(=O)O)c3OB2O)c2cc(F)c(C(=O)O)cc2Cl)C(=O)C1=O] | 604.7 | 604.5 |
| 150 | 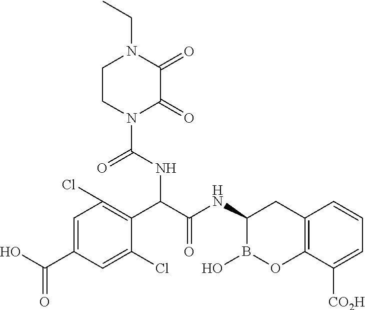 [SMILES: CCN1CCN(C(=O)NC(C(=O)N[C@H]2Cc3cccc(C(=O)O)c3OB2O)c2c(Cl)cc(C(=O)O)cc2Cl)C(=O)C1=O] | 621.2 | 621.0 |
| 151 | 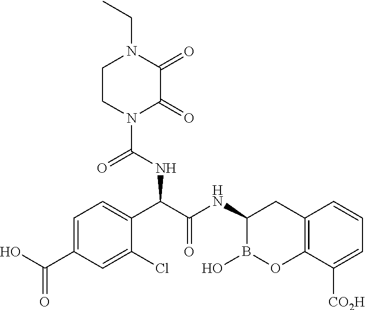 [SMILES: CCN1CCN(C(=O)N[C@@H](C(=O)N[C@H]2Cc3cccc(C(=O)O)c3OB2O)c2ccc(C(=O)O)cc2Cl)C(=O)C1=O] | 586.7 | 586.6 |
| 152 | 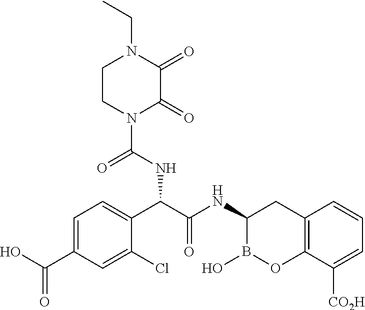 [SMILES: CCN1CCN(C(=O)N[C@H](C(=O)N[C@H]2Cc3cccc(C(=O)O)c3OB2O)c2ccc(C(=O)O)cc2Cl)C(=O)C1=O] | 586.7 | 586.6 |
| 153 | 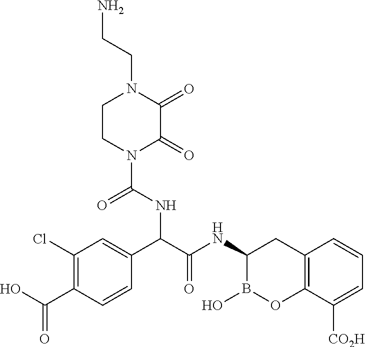 [SMILES: NCCN1CCN(C(=O)NC(C(=O)N[C@H]2Cc3cccc(C(=O)O)c3OB2O)c2ccc(C(=O)O)c(Cl)c2)C(=O)C1=O] | 601.8 | 601.6 |
| 154 | 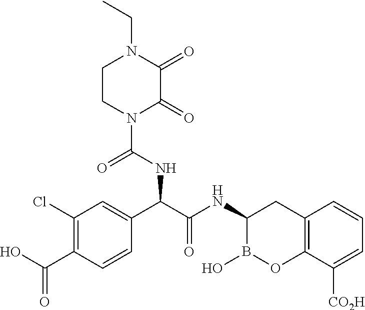 [SMILES: CCN1CCN(C(=O)N[C@@H](C(=O)N[C@H]2Cc3cccc(C(=O)O)c3OB2O)c2ccc(C(=O)O)c(Cl)c2)C(=O)C1=O] | 586.7 | 586.6 |
| 155 | 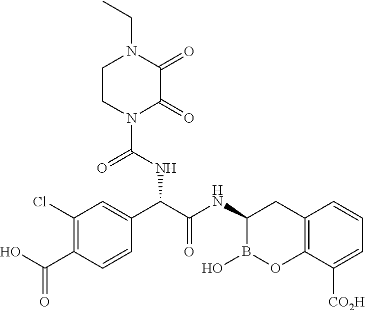 [SMILES: CCN1CCN(C(=O)N[C@H](C(=O)N[C@H]2Cc3cccc(C(=O)O)c3OB2O)c2ccc(C(=O)O)c(Cl)c2)C(=O)C1=O] | 586.7 | 586.6 |
| 156 | 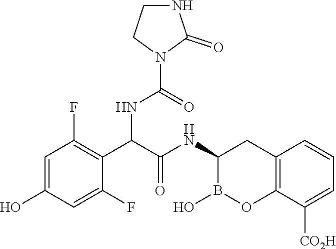 [SMILES: O=C(O)c1cccc2c1OB(O)[C@@H](NC(=O)C(NC(=O)N1CCNC1=O)c1c(F)cc(O)cc1F)C2] | 504.2 | 505.0 |
| 157 | 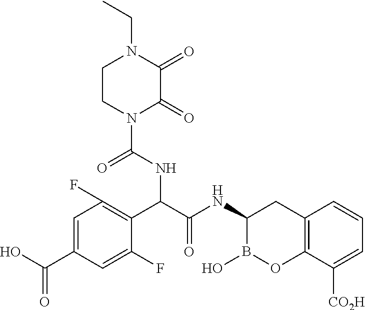 [SMILES: CCN1CCN(C(=O)NC(C(=O)N[C@H]2Cc3cccc(C(=O)O)c3OB2O)c2c(F)cc(C(=O)O)cc2F)C(=O)C1=O] | 588.3 | 589.1 |
| 158 | 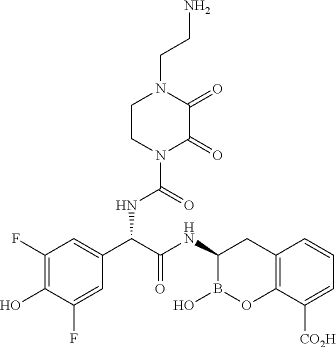 [SMILES: NCCN1CCN(C(=O)N[C@H](C(=O)N[C@H]2Cc3cccc(C(=O)O)c3OB2O)c2cc(F)c(O)c(F)c2)C(=O)C1=O] | 575.3 | 576.1 |
| 159 | 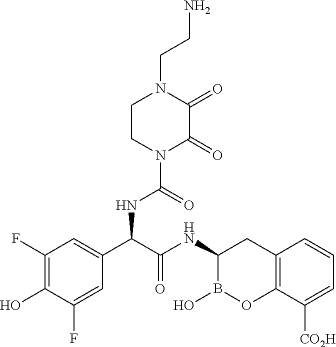 [SMILES: NCCN1CCN(C(=O)N[C@@H](C(=O)N[C@H]2Cc3cccc(C(=O)O)c3OB2O)c2cc(F)c(O)c(F)c2)C(=O)C1=O] | 575.3 | 576.1 |
| 160 | 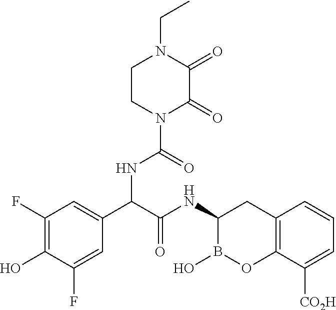 [SMILES: CCN1CCN(C(=O)NC(C(=O)N[C@H]2Cc3cccc(C(=O)O)c3OB2O)c2cc(F)c(O)c(F)c2)C(=O)C1=O] | 560.3 | 561.0 |
| 161 | 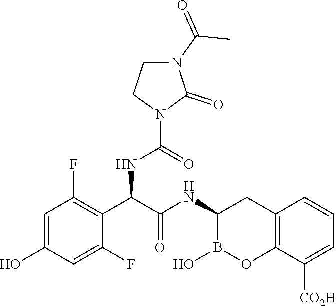 [SMILES: CC(=O)N1CCN(C(=O)N[C@@H](C(=O)N[C@H]2Cc3cccc(C(=O)O)c3OB2O)c2c(F)cc(O)cc2F)C1=O] | 546.2 | 547.0 |
| 162 | 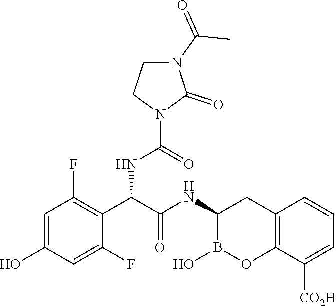 [SMILES: CC(=O)N1CCN(C(=O)N[C@H](C(=O)N[C@H]2Cc3cccc(C(=O)O)c3OB2O)c2c(F)cc(O)cc2F)C1=O] | 546.2 | 547.1 |
| 163 | 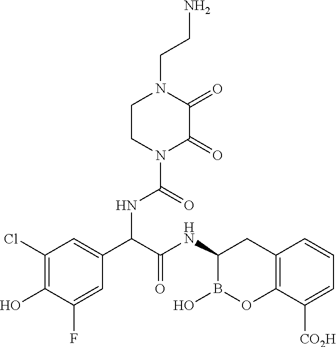 [SMILES: NCCN1CCN(C(=O)NC(C(=O)N[C@H]2Cc3cccc(C(=O)O)c3OB2O)c2cc(F)c(O)c(Cl)c2)C(=O)C1=O] | 591.7 | 591.7 |
| 164 | 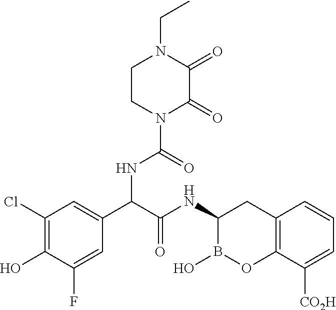 [SMILES: CCN1CCN(C(=O)NC(C(=O)N[C@H]2Cc3cccc(C(=O)O)c3OB2O)c2cc(F)c(O)c(Cl)c2)C(=O)C1=O] | 576.7 | 576.7 |
| 165 | 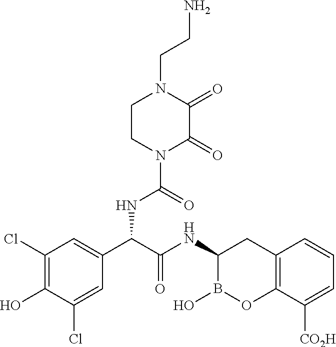 [SMILES: NCCN1CCN(C(=O)N[C@H](C(=O)N[C@H]2Cc3cccc(C(=O)O)c3OB2O)c2cc(Cl)c(O)c(Cl)c2)C(=O)C1=O] | 608.2 | 607.7 |
| 166 | 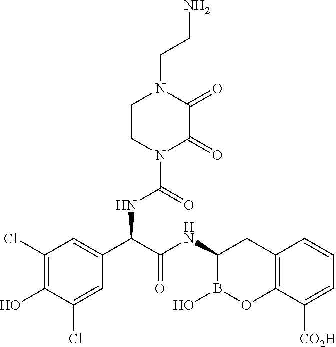 [SMILES: NCCN1CCN(C(=O)N[C@@H](C(=O)N[C@H]2Cc3cccc(C(=O)O)c3OB2O)c2cc(Cl)c(O)c(Cl)c2)C(=O)C1=O] | 608.2 | 607.7 |
| 167 | 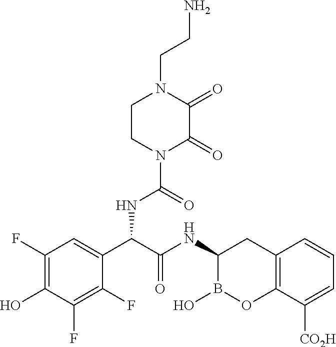 [SMILES: NCCN1CCN(C(=O)N[C@H](C(=O)N[C@H]2Cc3cccc(C(=O)O)c3OB2O)c2cc(F)c(O)c(F)c2F)C(=O)C1=O] | 593.3 | 593.7 |
| 168 | 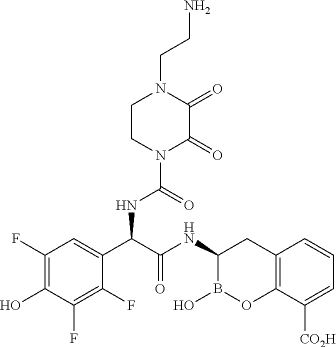 [SMILES: NCCN1CCN(C(=O)N[C@@H](C(=O)N[C@H]2Cc3cccc(C(=O)O)c3OB2O)c2cc(F)c(O)c(F)c2F)C(=O)C1=O] | 593.3 | 593.8 |
| 169 | 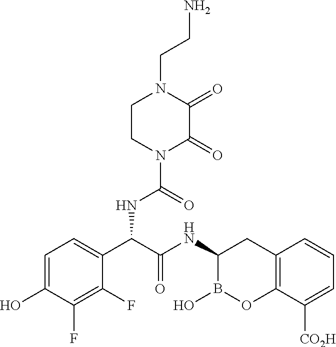 [SMILES: NCCN1CCN(C(=O)N[C@H](C(=O)N[C@H]2Cc3cccc(C(=O)O)c3OB2O)c2ccc(O)c(F)c2F)C(=O)C1=O] | 575.3 | 575.8 |
| 170 | 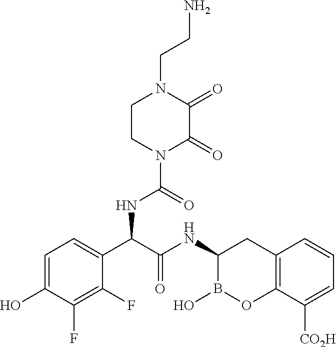 [SMILES: NCCN1CCN(C(=O)N[C@@H](C(=O)N[C@H]2Cc3cccc(C(=O)O)c3OB2O)c2ccc(O)c(F)c2F)C(=O)C1=O] | 575.3 | 575.8 |
| 171 | 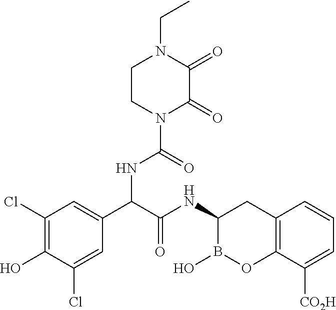 [SMILES: CCN1CCN(C(=O)NC(C(=O)N[C@H]2Cc3cccc(C(=O)O)c3OB2O)c2cc(Cl)c(O)c(Cl)c2)C(=O)C1=O] | 593.2 | 592.7 |
| 172 | 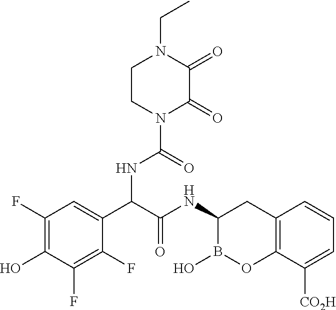 [SMILES: CCN1CCN(C(=O)NC(C(=O)N[C@H]2Cc3cccc(C(=O)O)c3OB2O)c2cc(F)c(O)c(F)c2F)C(=O)C1=O] | 578.3 | 578.8 |
| 173 | 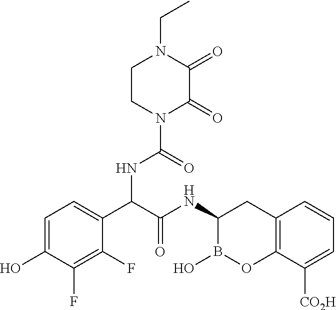 [SMILES: CCN1CCN(C(=O)NC(C(=O)N[C@H]2Cc3cccc(C(=O)O)c3OB2O)c2ccc(O)c(F)c2F)C(=O)C1=O] | 560.3 | 560.8 |
| 174 | 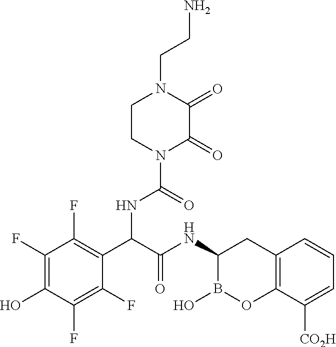 [SMILES: NCCN1CCN(C(=O)NC(C(=O)N[C@H]2Cc3cccc(C(=O)O)c3OB2O)c2c(F)c(F)c(O)c(F)c2F)C(=O)C1=O] | 611.3 | 612.0 |
| 175 | 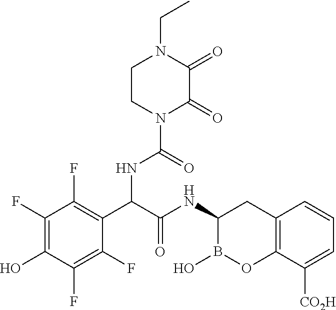 [SMILES: CCN1CCN(C(=O)NC(C(=O)N[C@H]2Cc3cccc(C(=O)O)c3OB2O)c2c(F)c(F)c(O)c(F)c2F)C(=O)C1=O] | 596.3 | 597.2 |
| 176 | 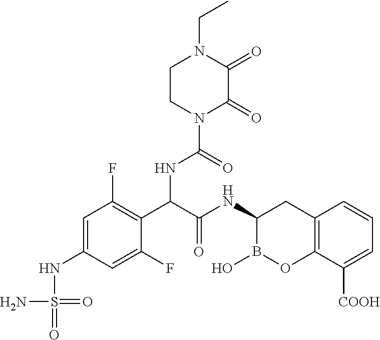 [SMILES: CCN1CCN(C(=O)NC(C(=O)N[C@H]2Cc3cccc(C(=O)O)c3OB2O)c2c(F)cc(NS(N)(=O)=O)cc2F)C(=O)C1=O] | 638.4 | 639 |
| 177 | 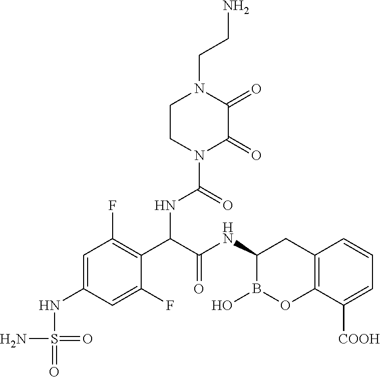 [SMILES: NCCN1CCN(C(=O)NC(C(=O)N[C@H]2Cc3cccc(C(=O)O)c3OB2O)c2c(F)cc(NS(N)(=O)=O)cc2F)C(=O)C1=O] | 653.4 | 654 |
| 178 | 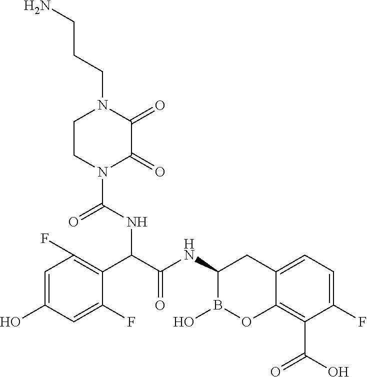 [SMILES: NCCCN1CCN(C(=O)NC(C(=O)N[C@H]2Cc3ccc(F)c(C(=O)O)c3OB2O)c2c(F)cc(O)cc2F)C(=O)C1=O] | 607.3 | 608.2 |
| 179 | 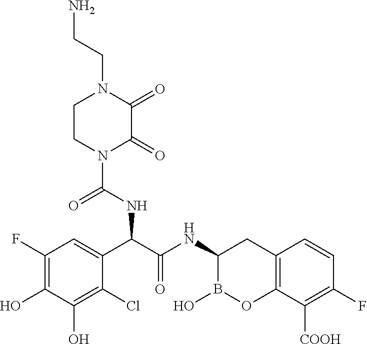 [SMILES: NCCN1CCN(C(=O)N[C@@H](C(=O)N[C@H]2Cc3ccc(F)c(C(=O)O)c3OB2O)c2cc(F)c(O)c(O)c2Cl)C(=O)C1=O] | 625.7 | 626 |
| 180 | 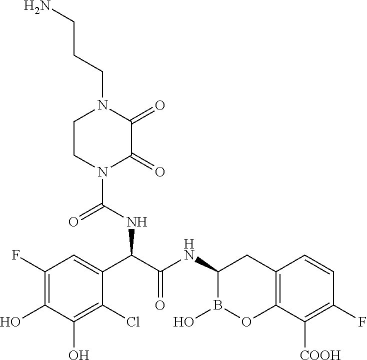 [SMILES: NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O] | 639.8 | 640 |
| 181 | 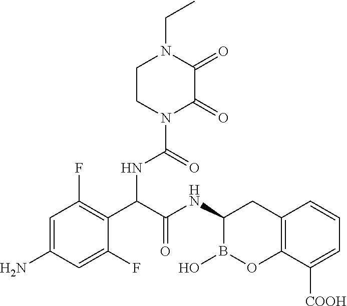 [SMILES: CCN1CCN(C(=O)NC(C(=O)N[C@H]2Cc3cccc(C(=O)O)c3OB2O)c2c(F)cc(N)cc2F)C(=O)C1=O] | 559.3 | 660 |
| 182 | 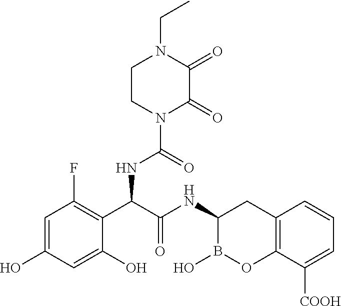 [SMILES: CCN1CCN(C(=O)N[C@@H](C(=O)N[C@H]2Cc3cccc(C(=O)O)c3OB2O)c2c(O)cc(O)cc2F)C(=O)C1=O] | 558.3 | 659 |
| 183 | 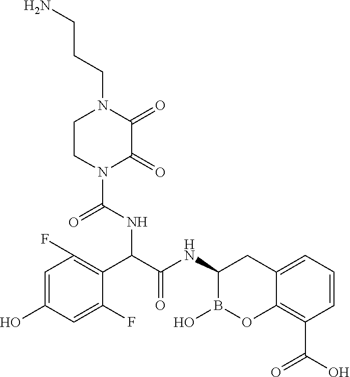 [SMILES: NCCCN1CCN(C(=O)NC(C(=O)N[C@H]2Cc3cccc(C(=O)O)c3OB2O)c2c(F)cc(O)cc2F)C(=O)C1=O] | 589.3 | 590.2 |
| 184 | 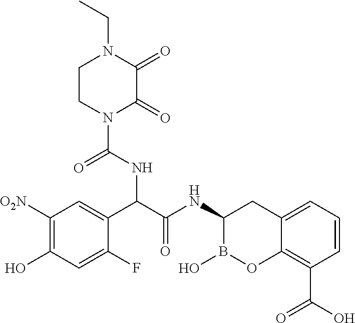 [SMILES: CCN1CCN(C(=O)NC(C(=O)N[C@H]2Cc3cccc(C(=O)O)c3OB2O)c2cc([N+](=O)[O-])c(O)cc2F)C(=O)C1=O] | 587.3 | 588.2 |
| 185 | 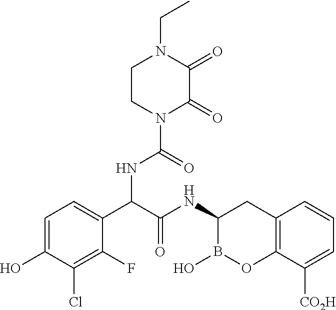 [SMILES: CCN1CCN(C(=O)NC(C(=O)N[C@H]2Cc3cccc(C(=O)O)c3OB2O)c2ccc(O)c(Cl)c2F)C(=O)C1=O] | 576.7 | 577.2 |
| 186 | 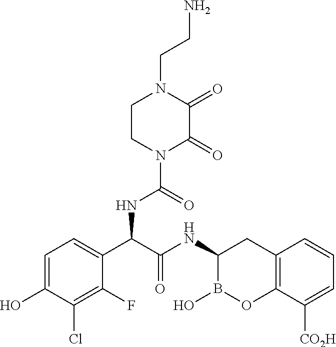 [SMILES: NCCN1CCN(C(=O)N[C@@H](C(=O)N[C@H]2Cc3cccc(C(=O)O)c3OB2O)c2ccc(O)c(Cl)c2F)C(=O)C1=O] | 591.7 | 591.9 |
| 187 | 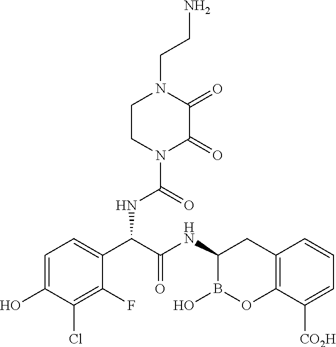 [SMILES: NCCN1CCN(C(=O)N[C@H](C(=O)N[C@H]2Cc3cccc(C(=O)O)c3OB2O)c2ccc(O)c(Cl)c2F)C(=O)C1=O] | 591.7 | 591.9 |
| 188 | 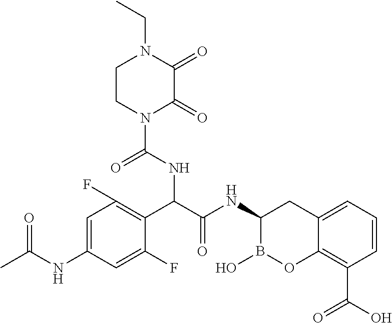 [SMILES: CCN1CCN(C(=O)NC(C(=O)N[C@H]2Cc3cccc(C(=O)O)c3OB2O)c2c(F)cc(NC(C)=O)cc2F)C(=O)C1=O] | 601.3 | 602.2 |
| 189 | 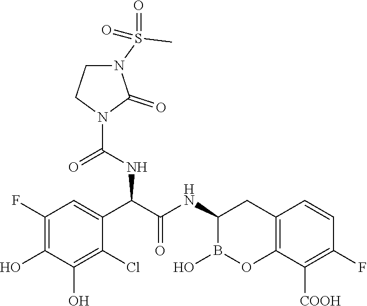 [SMILES: CS(=O)(=O)N1CCN(C(=O)N[C@@H](C(=O)N[C@H]2Cc3ccc(F)c(C(=O)O)c3OB2O)c2cc(F)c(O)c(O)c2Cl)C1=O] | 632.7 | 633 |
| 190 | 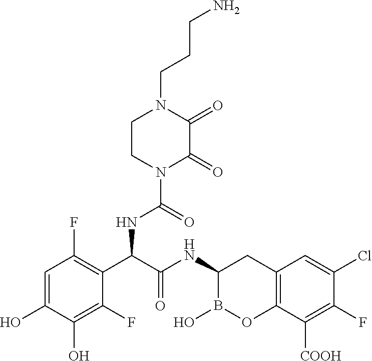 [SMILES: NCCCN1CCN(C(=O)N[C@@H](C(=O)N[C@H]2Cc3cc(Cl)c(F)c(C(=O)O)c3OB2O)c2c(F)cc(O)c(O)c2F)C(=O)C1=O] | 674.2 | 674 |
| 191 | 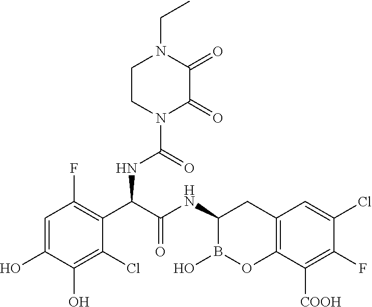 [SMILES: CCN1CCN(C(=O)N[C@@H](C(=O)N[C@H]2Cc3cc(Cl)c(F)c(C(=O)O)c3OB2O)c2c(F)cc(O)c(O)c2Cl)C(=O)C1=O] | 645.2 | 645 |
| 192 | 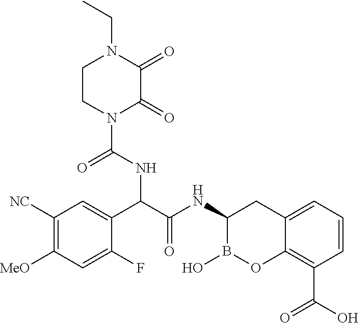 [SMILES: CCN1CCN(C(=O)NC(C(=O)N[C@H]2Cc3cccc(C(=O)O)c3OB2O)c2cc(C#N)c(OC)cc2F)C(=O)C1=O] | 581.3 | 582.2 |
| 193 | 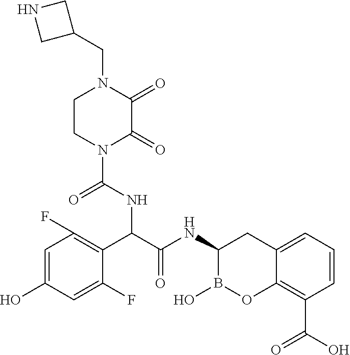 [SMILES: O=C(O)c1cccc2c1OB(O)[C@@H](NC(=O)C(NC(=O)N1CCN(CC3CNC3)C(=O)C1=O)c1c(F)cc(O)cc1F)C2] | 601.3 | 602.2 |
| 194 | 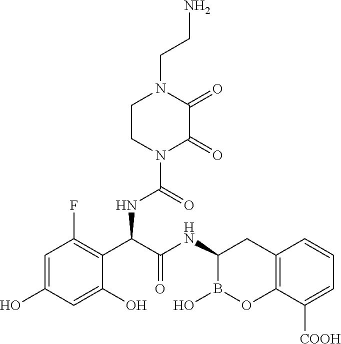 [SMILES: NCCN1CCN(C(=O)N[C@@H](C(=O)N[C@H]2Cc3cccc(C(=O)O)c3OB2O)c2c(O)cc(O)cc2F)C(=O)C1=O] | 573.3 | 574 |
| 195 | 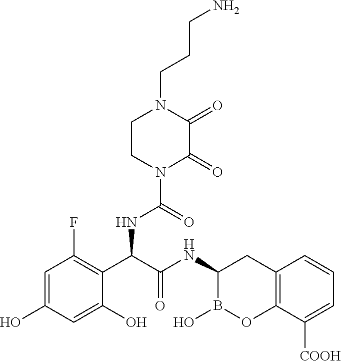 [SMILES: NCCCN1CCN(C(=O)N[C@@H](C(=O)N[C@H]2Cc3cccc(C(=O)O)c3OB2O)c2c(O)cc(O)cc2F)C(=O)C1=O] | 587.3 | 588 |
| 196 | 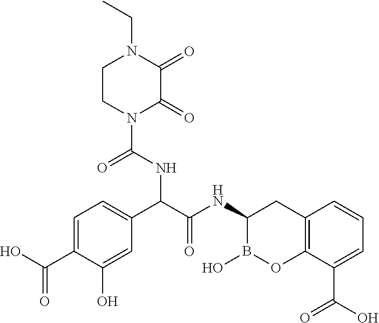 [SMILES: CCN1CCN(C(=O)NC(C(=O)N[C@H]2Cc3cccc(C(=O)O)c3OB2O)c2ccc(C(=O)O)c(O)c2)C(=O)C1=O] | 568.3 | 569.2 |
| 197 | 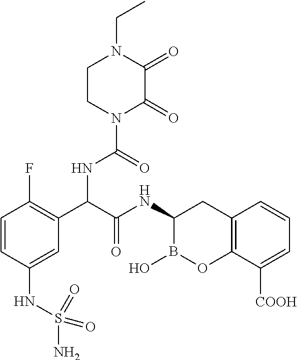 [SMILES: CCN1CCN(C(=O)NC(C(=O)N[C@H]2Cc3cccc(C(=O)O)c3OB2O)c2cc(NS(N)(=O)=O)ccc2F)C(=O)C1=O] | 620.4 | 621 |
| 198 | 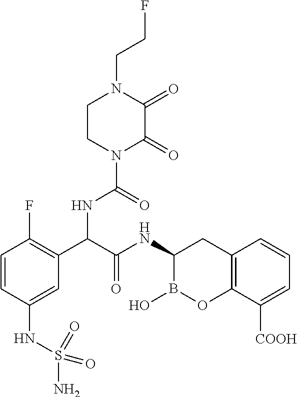 [SMILES: NS(=O)(=O)Nc1ccc(F)c(C(NC(=O)N2CCN(CCF)C(=O)C2=O)C(=O)N[C@H]2Cc3cccc(C(=O)O)c3OB2O)c1] | 638.4 | 639 |
| 199 | 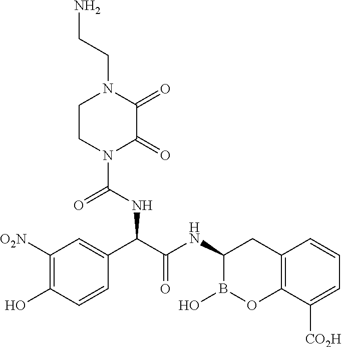 [SMILES: NCCN1CCN(C(=O)N[C@@H](C(=O)N[C@H]2Cc3cccc(C(=O)O)c3OB2O)c2ccc(O)c([N+](=O)[O-])c2)C(=O)C1=O] | 584.3 | 584.7 |
| 200 | 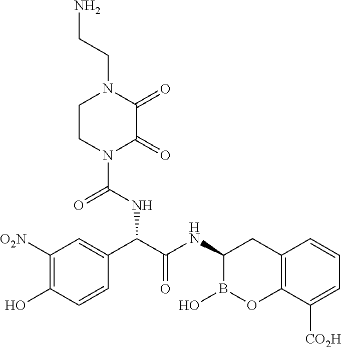 [SMILES: NCCN1CCN(C(=O)N[C@H](C(=O)N[C@H]2Cc3cccc(C(=O)O)c3OB2O)c2ccc(O)c([N+](=O)[O-])c2)C(=O)C1=O] | 584.3 | 584.6 |
| 201 | 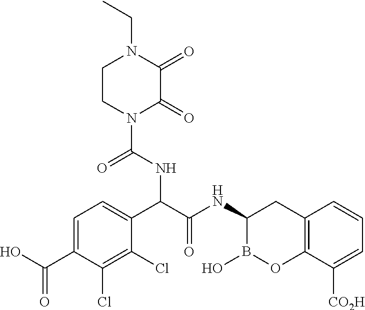 [SMILES: CCN1CCN(C(=O)NC(C(=O)N[C@H]2Cc3cccc(C(=O)O)c3OB2O)c2ccc(C(=O)O)c(Cl)c2Cl)C(=O)C1=O] | 621.2 | 620.7 |
| 202 | 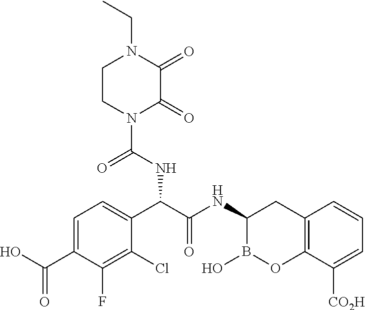 [SMILES: CCN1CCN(C(=O)N[C@H](C(=O)N[C@H]2Cc3cccc(C(=O)O)c3OB2O)c2ccc(C(=O)O)c(F)c2Cl)C(=O)C1=O] | 604.7 | 604.7 |
| 203 | 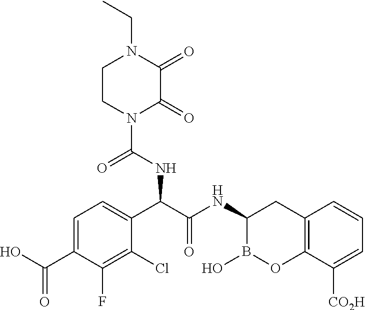 [SMILES: CCN1CCN(C(=O)N[C@@H](C(=O)N[C@H]2Cc3cccc(C(=O)O)c3OB2O)c2ccc(C(=O)O)c(F)c2Cl)C(=O)C1=O] | 604.7 | 604.7 |
| 204 | 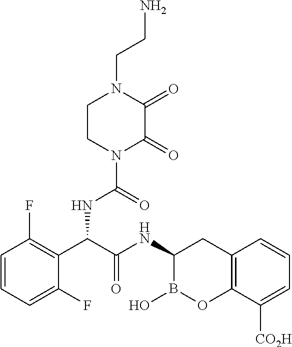 [SMILES: NCCN1CCN(C(=O)N[C@H](C(=O)N[C@H]2Cc3cccc(C(=O)O)c3OB2O)c2c(F)cccc2F)C(=O)C1=O] | 559.3 | 559.8 |
| 205 | 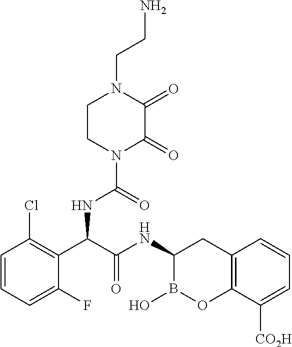 [SMILES: NCCN1CCN(C(=O)N[C@@H](C(=O)N[C@H]2Cc3cccc(C(=O)O)c3OB2O)c2c(F)cccc2Cl)C(=O)C1=O] | 575.7 | 575.8 |
| 206 | 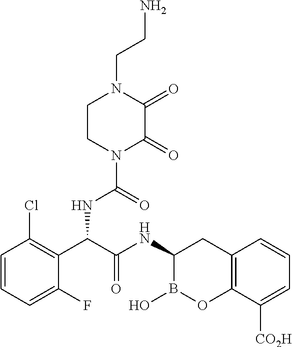 [SMILES: NCCN1CCN(C(=O)N[C@H](C(=O)N[C@H]2Cc3cccc(C(=O)O)c3OB2O)c2c(F)cccc2Cl)C(=O)C1=O] | 575.7 | 575.7 |
| 207 | 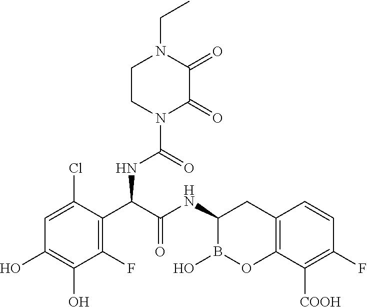 [SMILES: CCN1CCN(C(=O)N[C@@H](C(=O)N[C@H]2Cc3ccc(F)c(C(=O)O)c3OB2O)c2c(Cl)cc(O)c(O)c2F)C(=O)C1=O] | 610.7 | 611 |
| 208 | 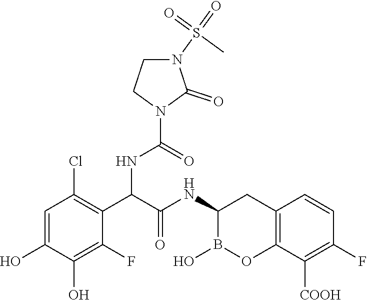 [SMILES: CS(=O)(=O)N1CCN(C(=O)NC(C(=O)N[C@H]2Cc3ccc(F)c(C(=O)O)c3OB2O)c2c(Cl)cc(O)c(O)c2F)C1=O] | 632.7 | 633 |
| 209 | 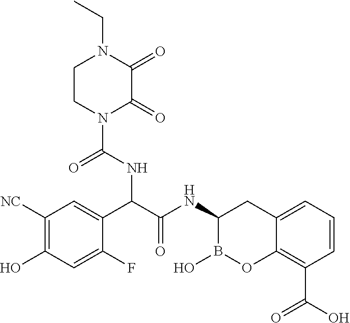 [SMILES: CCN1CCN(C(=O)NC(C(=O)N[C@H]2Cc3cccc(C(=O)O)c3OB2O)c2cc(C#N)c(O)cc2F)C(=O)C1=O] | 567.3 | 568.2 |
| 210 | 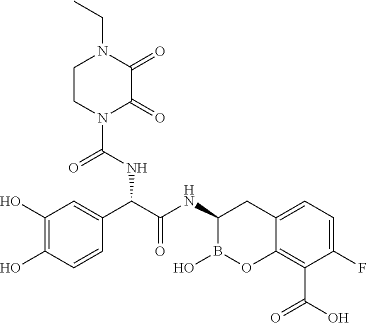 [SMILES: CCN1CCN(C(=O)N[C@H](C(=O)N[C@H]2Cc3ccc(F)c(C(=O)O)c3OB2O)c2ccc(O)c(O)c2)C(=O)C1=O] | 558.3 | 558.6 |
| 211 | 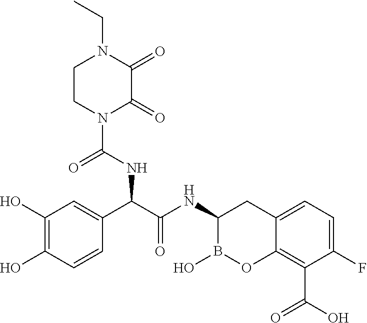 [SMILES: CCN1CCN(C(=O)N[C@@H](C(=O)N[C@H]2Cc3ccc(F)c(C(=O)O)c3OB2O)c2ccc(O)c(O)c2)C(=O)C1=O] | 558.3 | 540.6 [MH⁺ − 18] 558.6 |
| 212 | 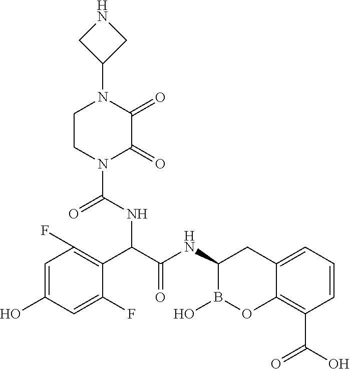 [SMILES: O=C(O)c1cccc2c1OB(O)[C@@H](NC(=O)C(NC(=O)N1CCN(C3CNC3)C(=O)C1=O)c1c(F)cc(O)cc1F)C2] | 587.3 | 588.2 |
| 213 | 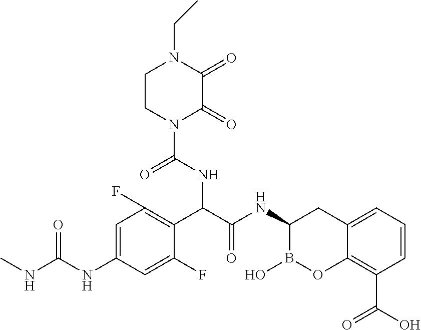 [SMILES: CCN1CCN(C(=O)NC(C(=O)N[C@H]2Cc3cccc(C(=O)O)c3OB2O)c2c(F)cc(NC(=O)NC)cc2F)C(=O)C1=O] | 616.3 | 617.2 |
| 215 | 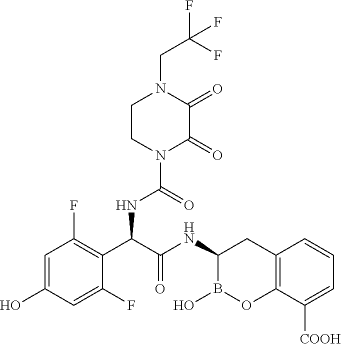 [SMILES: O=C(O)c1cccc2c1OB(O)[C@@H](NC(=O)[C@H](NC(=O)N1CCN(CC(F)(F)F)C(=O)C1=O)c1c(F)cc(O)cc1F)C2] | 614.2 | 615 |
| 216 | 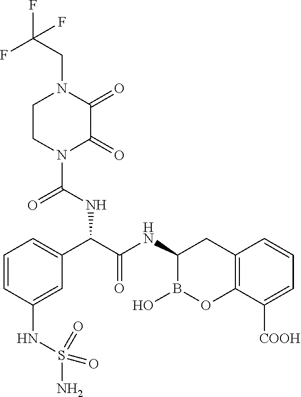 [SMILES: NS(=O)(=O)Nc1cccc([C@H](NC(=O)N2CCN(CC(F)(F)F)C(=O)C2=O)C(=O)N[C@H]2Cc3cccc(C(=O)O)c3OB2O)c1] | 656.4 | 657 |
| 217 | 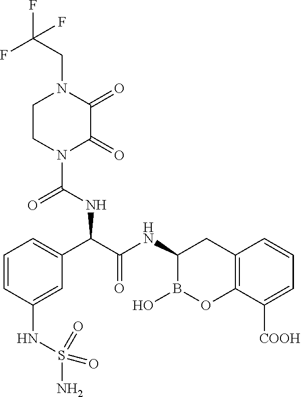 [SMILES: NS(=O)(=O)Nc1cccc([C@@H](NC(=O)N2CCN(CC(F)(F)F)C(=O)C2=O)C(=O)N[C@H]2Cc3cccc(C(=O)O)c3OB2O)c1] | 656.4 | 657 |
| 218 | 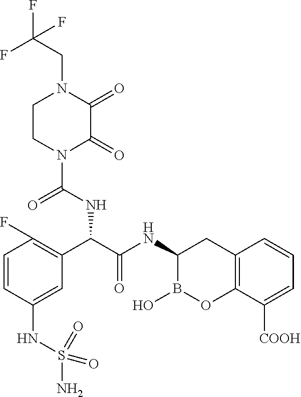 [SMILES: NS(=O)(=O)Nc1ccc(F)c([C@H](NC(=O)N2CCN(CC(F)(F)F)C(=O)C2=O)C(=O)N[C@H]2Cc3cccc(C(=O)O)c3OB2O)c1] | 674.3 | 675 |
| 219 | 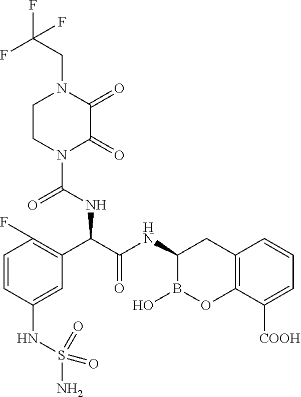 [SMILES: NS(=O)(=O)Nc1ccc(F)c([C@@H](NC(=O)N2CCN(CC(F)(F)F)C(=O)C2=O)C(=O)N[C@H]2Cc3cccc(C(=O)O)c3OB2O)c1] | 674.3 | 675 |
| 220 | 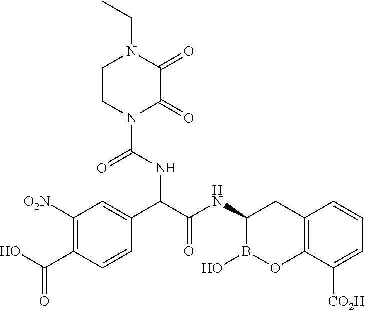 [SMILES: CCN1CCN(C(=O)NC(C(=O)N[C@H]2Cc3cccc(C(=O)O)c3OB2O)c2ccc(C(=O)O)c([N+](=O)[O-])c2)C(=O)C1=O] | 597.3 | 597.6 |
| 221 | 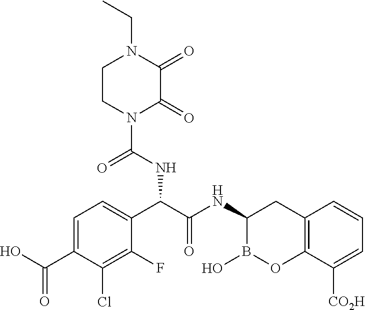 [SMILES: CCN1CCN(C(=O)N[C@H](C(=O)N[C@H]2Cc3cccc(C(=O)O)c3OB2O)c2ccc(C(=O)O)c(Cl)c2F)C(=O)C1=O] | 604.7 | 604.5 |
| 222 | 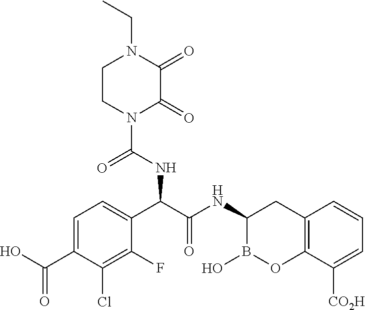 [SMILES: CCN1CCN(C(=O)N[C@@H](C(=O)N[C@H]2Cc3cccc(C(=O)O)c3OB2O)c2ccc(C(=O)O)c(Cl)c2F)C(=O)C1=O] | 604.7 | 604.5 |
| 223 | 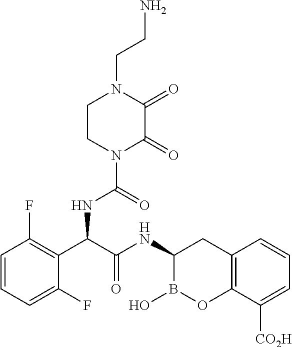 [SMILES: NCCN1CCN(C(=O)N[C@@H](C(=O)N[C@H]2Cc3cccc(C(=O)O)c3OB2O)c2c(F)cccc2F)C(=O)C1=O] | 559.3 | 560.0 |
| 224 | 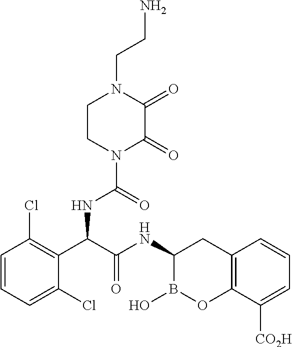 [SMILES: NCCN1CCN(C(=O)N[C@@H](C(=O)N[C@H]2Cc3cccc(C(=O)O)c3OB2O)c2c(Cl)cccc2Cl)C(=O)C1=O] | 592.2 | 591.5 |
| 225 | 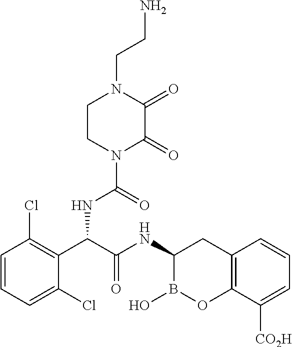 [SMILES: NCCN1CCN(C(=O)N[C@H](C(=O)N[C@H]2Cc3cccc(C(=O)O)c3OB2O)c2c(Cl)cccc2Cl)C(=O)C1=O] | 592.2 | 591.5 |
| 226 | 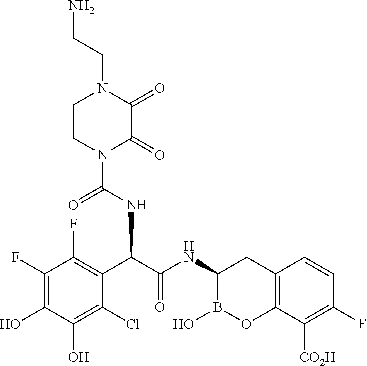 [SMILES: NCCN1CCN(C(=O)N[C@@H](C(=O)N[C@H]2Cc3ccc(F)c(C(=O)O)c3OB2O)c2c(F)c(F)c(O)c(O)c2Cl)C(=O)C1=O] | 643.7 | 644.1 |
| 227 | 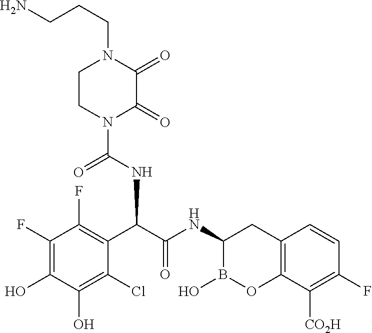 [SMILES: NCCCN1CCN(C(=O)N[C@@H](C(=O)N[C@H]2Cc3ccc(F)c(C(=O)O)c3OB2O)c2c(F)c(F)c(O)c(O)c2Cl)C(=O)C1=O] | 657.7 | 658.1 |
| 228 | 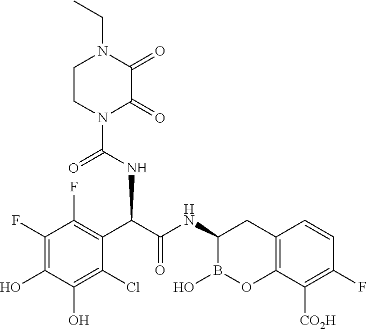 [SMILES: CCN1CCN(C(=O)N[C@@H](C(=O)N[C@H]2Cc3ccc(F)c(C(=O)O)c3OB2O)c2c(F)c(F)c(O)c(O)c2Cl)C(=O)C1=O] | 628.7 | 629.1 |
| 229 | 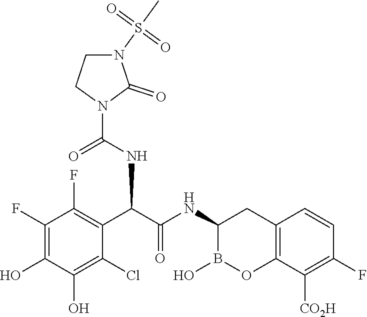 [SMILES: CS(=O)(=O)N1CCN(C(=O)N[C@@H](C(=O)N[C@H]2Cc3ccc(F)c(C(=O)O)c3OB2O)c2c(F)c(F)c(O)c(O)c2Cl)C1=O] | 650.7 | 651.1 |
| 230 | 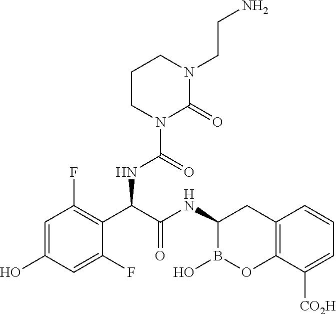 [SMILES: NCCN1CCCN(C(=O)N[C@@H](C(=O)N[C@H]2Cc3cccc(C(=O)O)c3OB2O)c2c(F)cc(O)cc2F)C1=O] | 561.3 | 561.7 |
| 231 | 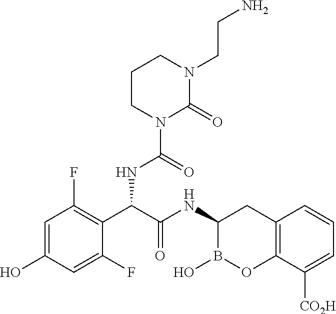 [SMILES: NCCN1CCCN(C(=O)N[C@H](C(=O)N[C@H]2Cc3cccc(C(=O)O)c3OB2O)c2c(F)cc(O)cc2F)C1=O] | 561.3 | 561.6 |
| 232 | 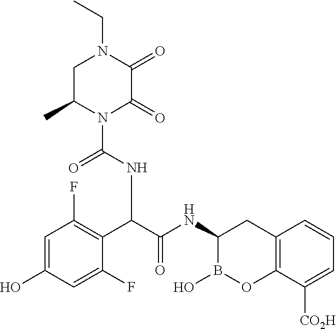 [SMILES: CCN1C[C@H](C)N(C(=O)NC(C(=O)N[C@H]2Cc3cccc(C(=O)O)c3OB2O)c2c(F)cc(O)cc2F)C(=O)C1=O] | 574.3 | 575.0 |
| 233 | 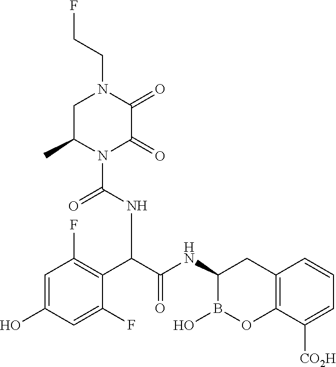 [SMILES: C[C@H]1CN(CCF)C(=O)C(=O)N1C(=O)NC(C(=O)N[C@H]1Cc2cccc(C(=O)O)c2OB1O)c1c(F)cc(O)cc1F] | 592.3 | 592.6 |
| 234 | 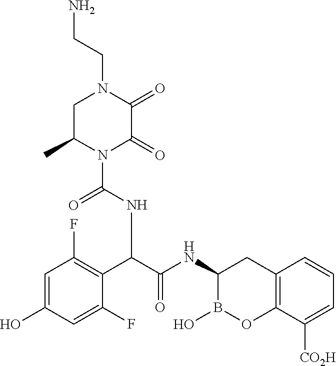 [SMILES: C[C@H]1CN(CCN)C(=O)C(=O)N1C(=O)NC(C(=O)N[C@H]1Cc2cccc(C(=O)O)c2OB1O)c1c(F)cc(O)cc1F] | 589.3 | 589.6 |
| 235 | 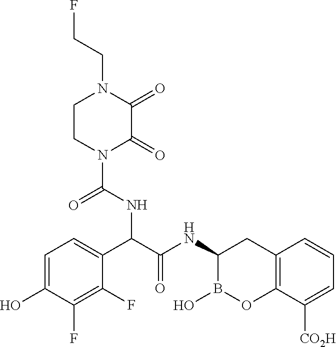 [SMILES: O=C(O)c1cccc2c1OB(O)[C@@H](NC(=O)C(NC(=O)N1CCN(CCF)C(=O)C1=O)c1ccc(O)c(F)c1F)C2] | 578.3 | 578.6 |
| 236 | 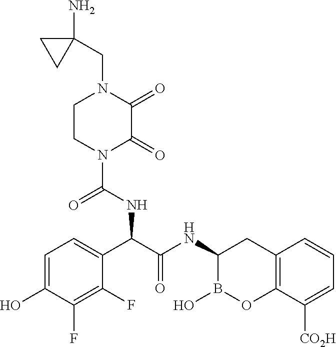 [SMILES: NC1(CN2CCN(C(=O)N[C@@H](C(=O)N[C@H]3Cc4cccc(C(=O)O)c4OB3O)c3ccc(O)c(F)c3F)C(=O)C2=O)CC1] | 601.3 | 601.6 |
| 237 | 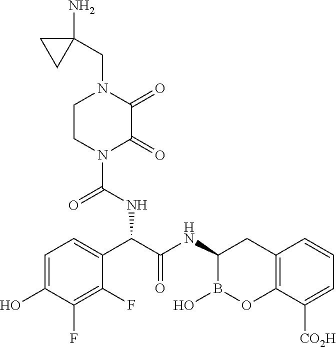 [SMILES: NC1(CN2CCN(C(=O)N[C@H](C(=O)N[C@H]3Cc4cccc(C(=O)O)c4OB3O)c3ccc(O)c(F)c3F)C(=O)C2=O)CC1] | 601.3 | 601.6 |
| 238 | 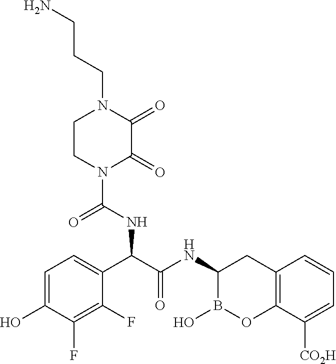 [SMILES: NCCCN1CCN(C(=O)N[C@@H](C(=O)N[C@H]2Cc3cccc(C(=O)O)c3OB2O)c2ccc(O)c(F)c2F)C(=O)C1=O] | 589.3 | 589.8 |
| 239 | 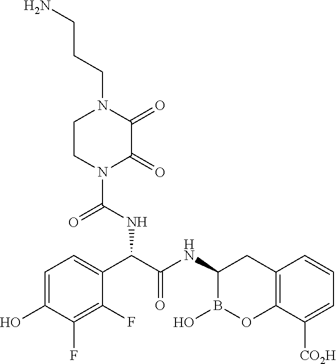 [SMILES: NCCCN1CCN(C(=O)N[C@H](C(=O)N[C@H]2Cc3cccc(C(=O)O)c3OB2O)c2ccc(O)c(F)c2F)C(=O)C1=O] | 589.3 | 589.8 |
| 240 | 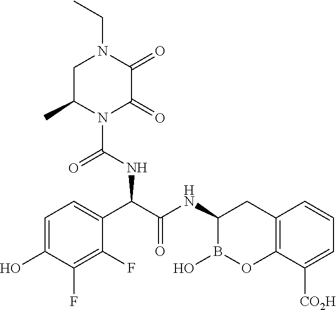 [SMILES: CCN1C[C@H](C)N(C(=O)N[C@@H](C(=O)N[C@H]2Cc3cccc(C(=O)O)c3OB2O)c2ccc(O)c(F)c2F)C(=O)C1=O] | 574.3 | 575.0 |
| 241 | 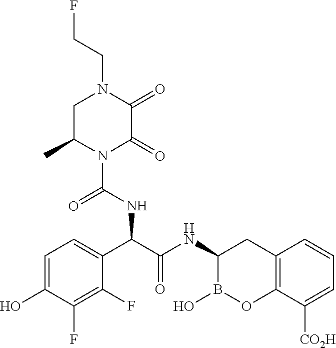 [SMILES: C[C@H]1CN(CCF)C(=O)C(=O)N1C(=O)N[C@@H](C(=O)N[C@H]1Cc2cccc(C(=O)O)c2OB1O)c1ccc(O)c(F)c1F] | 592.3 | 592.6 |
| 242 | 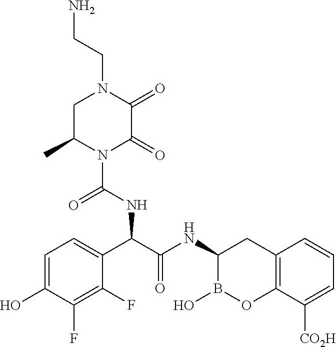 [SMILES: C[C@H]1CN(CCN)C(=O)C(=O)N1C(=O)N[C@@H](C(=O)N[C@H]1Cc2cccc(C(=O)O)c2OB1O)c1ccc(O)c(F)c1F] | 589.3 | 589.6 |
| 243 | 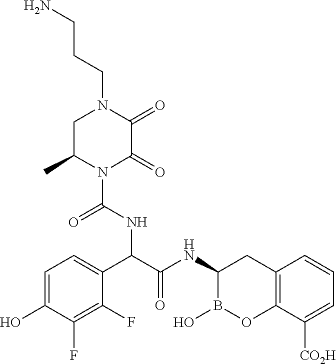 [SMILES: C[C@H]1CN(CCCN)C(=O)C(=O)N1C(=O)NC(C(=O)N[C@H]1Cc2cccc(C(=O)O)c2OB1O)c1ccc(O)c(F)c1F] | 603.3 | 604.2 |
| 244 | 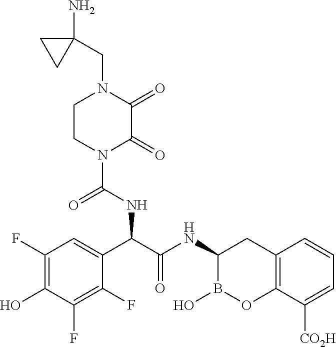 [SMILES: NC1(CN2CCN(C(=O)N[C@@H](C(=O)N[C@H]3Cc4cccc(C(=O)O)c4OB3O)c3cc(F)c(O)c(F)c3F)C(=O)C2=O)CC1] | 619.3 | 619.5 |
| 245 | 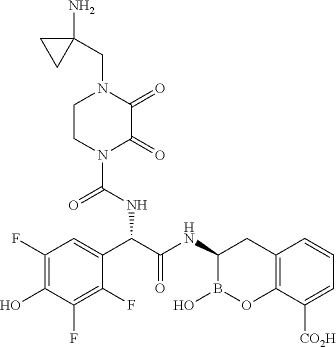 [SMILES: NC1(CN2CCN(C(=O)N[C@H](C(=O)N[C@H]3Cc4cccc(C(=O)O)c4OB3O)c3cc(F)c(O)c(F)c3F)C(=O)C2=O)CC1] | 619.3 | 619.5 |
| 246 | 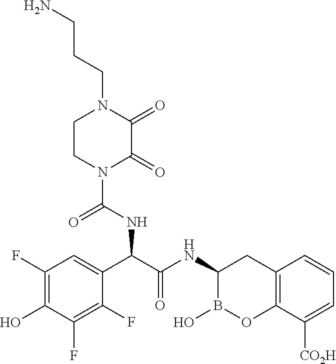 [SMILES: NCCCN1CCN(C(=O)N[C@@H](C(=O)N[C@H]2Cc3cccc(C(=O)O)c3OB2O)c2cc(F)c(O)c(F)c2F)C(=O)C1=O] | 607.3 | 607.6 |
| 247 | 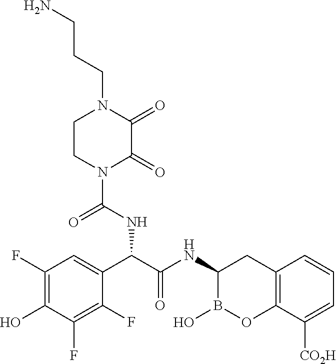 [SMILES: NCCCN1CCN(C(=O)N[C@H](C(=O)N[C@H]2Cc3cccc(C(=O)O)c3OB2O)c2cc(F)c(O)c(F)c2F)C(=O)C1=O] | 607.3 | 607.6 |
| 248 | 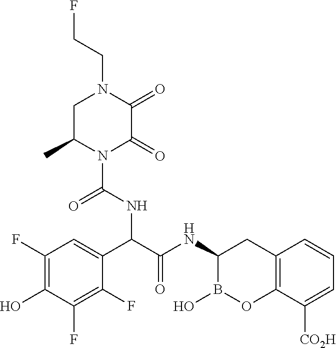 [SMILES: C[C@H]1CN(CCF)C(=O)C(=O)N1C(=O)NC(C(=O)N[C@H]1Cc2cccc(C(=O)O)c2OB1O)c1cc(F)c(O)c(F)c1F] | 610.3 | 610.5 |
| 249 | 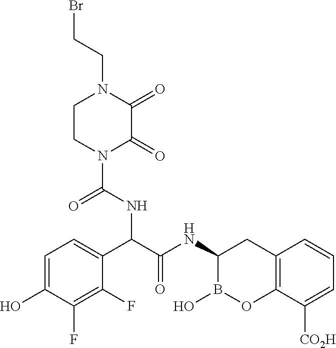 [SMILES: O=C(O)c1cccc2c1OB(O)[C@@H](NC(=O)C(NC(=O)N1CCN(CCBr)C(=O)C1=O)c1ccc(O)c(F)c1F)C2] | 639.2 | 638.4 |
| 250 | 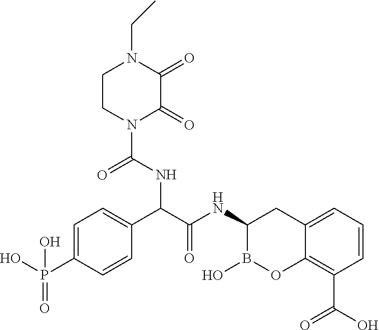 [SMILES: CCN1CCN(C(=O)NC(C(=O)N[C@H]2Cc3cccc(C(=O)O)c3OB2O)c2ccc(P(=O)(O)O)cc2)C(=O)C1=O] | 588.273 | 589.2 |
| 251 | 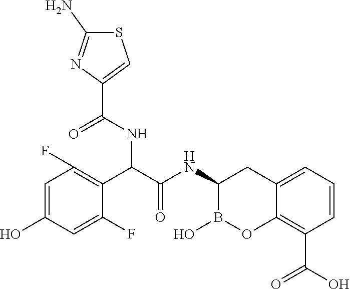 [SMILES: Nc1nc(C(=O)NC(C(=O)N[C@H]2Cc3cccc(C(=O)O)c3OB2O)c2c(F)cc(O)cc2F)cs1] | 518.255 | 519 |

### Example A1: Parenteral Composition

To prepare a parenteral pharmaceutical composition suitable for administration by injection, 100 mg of a compound disclosed herein, or a pharmaceutically acceptable salt, solvate, stereoisomer, tautomer, N-oxide, dimer, or trimer thereof, is dissolved in DMSO and then mixed with 10 ml of 0.9% sterile saline solution. The mixture is incorporated into a dosage unit suitable for administration by injection.

### Example A2: Oral Composition

To prepare a pharmaceutical composition for oral delivery, 400 mg of compound disclosed and the following ingredients are mixed intimately and pressed into single scored tablets.

### Tablet Formulation

| Ingredient | Quantity per tablet (mg) |
|---|---|
| compound | 400 |
| cornstarch | 50 |
| croscarmellose sodium | 25 |
| lactose | 120 |
| magnesium stearate | 5 |

The following ingredients are mixed intimately and loaded into a hard-shell gelatin capsule.

### Capsule Formulation

| Ingredient | Quantity per capsule (mg) |
|---|---|
| compound | 200 |
| lactose spray dried | 148 |
| magnesium stearate | 2 |

### BIOLOGICAL EXAMPLES

### Example I: Experimental Method for Penicillin-Binding Protein Binding Assays with Bocillin-FL Via Fluorescence Polarization

To determine the ability of boronic acid-based test PBP inhibitors to bind Penicillin Binding Proteins (PBPs), Bocillin-FL (fluorescently-labeled penicillin V; ThermoFisher Scientific) was used in a fluorescence polarization (FP) competition binding assay to assess inhibitor binding to PBP2, PBP3 or PBP4 from Escherichia coli (K12), PBP3 from Pseudomonas aeruginosa (PA01), PBP1a, PBP2 and PBP3 from Acinetobacter baumannii (ATCC 19606), and PBP2 from Neiserria gonorrheae (FA19). PBPs were cloned and purified as described previously (E. coli PBPs, King, D. T, et al., ACS Infectious Diseases 2015, 1, 175-184; P. aeruginosa PBP3, Han et. al., PNAS 2010, 107 (51), 22002-22007; A. baumannii PBPs, Penwell et. al., Antimicrob. Agents Chemother. 2015, 59 (3), 1680-1689; N. gonorrhoeae PBP2, Singh et. al., J Biol. Chem. 2019, 294 (38), 14020-14032). To establish assay conditions for competition binding, enzyme titration/saturation binding experiments were initially performed. Bocillin-FL was prepared at 0.2 μM in a buffer comprised of 50 mM Hepes (pH 8.0), 300 mM NaCl and 5% (v/v) glycerol for reactions with E. coli, P. aeruginosa and N. gonorrhoeae PBPs, and 25 mM Tris (pH 8.0), 200 mM NaCl and 10% (v/v) glycerol and 0.005% (v/v) Tween 20 for reactions with A. baumannii PBPs. Saturation binding was performed by mixing 40 μl of PBP solutions ranging in concentrations from 0-24 μM with 40 μl of the 0.2 μM Bocillin-FL solution, in individual wells of a black 384-well microplate. FP was measured immediately upon mixing (Excitation, 490 nm; Emission, 520 nm; g-factor, 0.96), using a Cytation3 (BioTek) microplate reader and measured continuously for up to 120 minutes. The FP response stabilized after 15 minutes for P. aeruginosa and A. baumannii PBP3, 30 minutes for E. coli PBP3 and PBP4, as well as for A. baumannii PBP1a and PBP2, 80 minutes for E. coli PBP2, and less than 1 minute for N. gonorrhoeae. In all instances, the FP signal showed a dose dependence on PBP concentration. The competition binding assay (80 μl final volume) was validated using beta-lactams and PBPs at final concentrations of: 1.5 μM, E. coli PBP3; 0.75 μM, P. aeruginosa PBP3; 1 μM A. baumannii PBP1a; 2.5 μM A. baumannii PBP2; 0.2 μM A. baumannii PBP3; and 0.25 M N. gonorrhoeae PBP2. Bocillin-FL was at 0.1 μM (0.05 μM with A. baumannii PBP1a) and beta-lactam concentrations ranged from 0-1000 μM. E. coli PBP3 was incubated with increasing concentrations of ampicillin or aztreonam in a black 384-well microplate (Corning) for 30 minutes, and PBP2 and PBP4 were likewise incubated with increasing concentrations of mecillinam and meropenem, respectively. P. aeruginosa PBP3 was incubated for 15 minutes with aztreonam, whereas A. baumannii PBP1a and PBP3 were incubated for 15 minutes with meropenem, and A. baumannii PBP2 for 15 minutes with mecillinam. Bocillin-FL was added and the FP immediately measured for up to 60 minutes (90 minutes for E. coli PBP2). For N. gonorrhoeae PBP2 assays, cefixime/ceftriaxone were mixed with Bocillin-FL, then enzyme was added and the FP immediately measured for up to 30 minutes. The beta-lactam potency was reported as the concentration of beta-lactam required to reduce the amount of PBP bound-Bocillin-FL by 50% (EC₅₀). The EC₅₀ for E. coli PBP3 with ampicillin was determined to be 1.4 μM, while that of the PBP3-specific beta-lactam aztreonam was determined to be 0.8 μM, and <0.5 μM for P. aeruginosa PBP3. The EC₅₀ for mecillinam with E. coli PBP2 was found to be 2.1 μM, and the EC₅₀ for meropenem with E. coli PBP4 was found to be <2 μM. EC₅₀s for meropenem with A. baumannii PBP1a and PBP3 were each determined to be 0.23 μM, while the EC₅₀ for mecillinam was 0.4 μM with A. baumannii PBP2. EC₅₀s for cefixime and ceftriaxone with N. gonorrhoeae PBP2 were 0.26 μM and 0.27 μM, respectively. Binding assays for boronic acid PBP inhibitors were performed in an identical fashion for the respective PBPs

Representative results for binding to E. coli PBP3 are shown in Table 2, where A represents a potency of >500 μM, B represents a potency between 30 μM and 500 μM inclusive, and C represents a potency of <30 μM. NT=Not Tested.

TABLE 2
Binding affinity to E. coli PBP3 by Exemplary
Compounds in fluorescence polarization competition
binding assay using Bocillin-FL.

| | E. coli K12 | | E. coli K12 | | E. coli K12 |
|---|---|---|---|---|---|
| Ex. | PBP3 Potency | Ex. | PBP3 Potency | Ex. | PBP3 Potency |
| 1 | C | 2 | C | 3 | C |
| 4 | C | 5 | C | 6 | B |
| 7 | B | 8 | B | 9 | B |
| 10 | B | 11 | C | 12 | B |
| 13 | C | 14 | C | 15 | C |
| 16 | C | 17 | B | 18 | C |
| 19 | C | 20 | C | 21 | C |
| 22 | C | 23 | C | 24 | B |
| 25 | B | 26 | C | 27 | C |
| 28 | B | 29 | C | 30 | C |
| 31 | C | 32 | A | 33 | NT |
| 34 | C | 35 | C | 36 | C |
| 37 | C | 38 | C | 39 | C |
| 40 | C | 41 | C | 42 | C |
| 43 | A | 44 | A | 45 | C |
| 46 | C | 47 | B | 48 | C |
| 49 | B | 50 | C | 51 | B |
| 52 | A | 53 | B | 54 | B |
| 55 | B | 56 | B | 57 | B |
| 58 | B | 59 | B | 60 | B |
| 61 | NT | 62 | A | 63 | A |
| 64 | A | 65 | A | 66 | A |
| 67 | B | 68 | B | 69 | C |
| 70 | B | 71 | A | 72 | C |
| 73 | C | 74 | C | 75 | B |
| 76 | C | 77 | B | 78 | B |
| 79 | B | 80 | C | 81 | A |
| 82 | B | 83 | B | 84 | C |
| 85 | B | 86 | C | 87 | C |
| 88 | B | 89 | B | 90 | C |
| 91 | C | 92 | A | 93 | C |
| 94 | C | 95 | C | 96 | B |
| 97 | C | 98 | C | 99 | B |
| 100 | B | 101 | C | 102 | B |
| 103 | C | 104 | C | 105 | C |
| 106 | B | 107 | B | 108 | B |
| 109 | C | 110 | B | 111 | C |
| 112 | B | 113 | C | 114 | B |
| 115 | B | 116 | C | 117 | B |
| 118 | B | 119 | C | 120 | B |
| 121 | C | 122 | C | 123 | C |
| 124 | C | 125 | A | 126 | C |
| 127 | C | 128 | B | 129 | C |
| 130 | C | 131 | C | 132 | B |
| 133 | B | 134 | B | 135 | C |
| 136 | B | 137 | C | 138 | C |
| 139 | C | 140 | C | 141 | C |
| 142 | B | 143 | C | 144 | C |
| 145 | C | 146 | C | 147 | C |
| 148 | C | 149 | B | 150 | B |
| 151 | C | 152 | B | 153 | C |
| 154 | C | 155 | C | 156 | C |
| 157 | C | 158 | B | 159 | C |
| 160 | C | 161 | C | 162 | B |
| 163 | C | 164 | C | 165 | B |
| 166 | C | 167 | B | 168 | C |
| 169 | B | 170 | C | 171 | C |
| 172 | C | 173 | C | 174 | C |
| 175 | C | 176 | C | 177 | B |
| 178 | C | 179 | C | 180 | C |
| 181 | C | 182 | C | 183 | C |
| 184 | C | 185 | C | 186 | C |
| 187 | B | 188 | C | 189 | C |
| 190 | C | 191 | C | 192 | B |
| 193 | C | 194 | C | 195 | C |
| 196 | C | 197 | C | 198 | C |
| 199 | C | 200 | A | 201 | C |
| 202 | B | 203 | C | 204 | A |
| 205 | B | 206 | A | 207 | C |
| 208 | C | 209 | C | 210 | A |
| 211 | C | 212 | C | 213 | C |
| 215 | C | 216 | B | 217 | C |
| 218 | B | 219 | C | 230 | B |
| 231 | B | 232 | C | 233 | C |
| 234 | C | 235 | C | 236 | C |
| 237 | B | | | | |

Representative results for binding to E. coli PBP2 are shown in Table 3, where A represents a potency of >500 μM, B represents a potency between 30 μM and 500 μM inclusive, and C represents a potency of <30 μM. NT=Not Tested.

TABLE 3
Binding affinity to E. coli PBP2 by Exemplary
Compounds in fluorescence polarization competition
binding assay using Bocillin-FL.

| | E. coli K12 PBP2 |
|---|---|
| Ex. | Potency (EC₅₀) |
| 54 | B |
| 55 | B |

Representative results for binding to E. coli PBP4 are shown in Table 4, where A represents a potency of >500 μM, B represents a potency between 30 μM and 500 μM inclusive, and C represents a potency of <30 μM. NT=Not Tested.

TABLE 4
Binding affinity to E. coli PBP4 by Exemplary
Compounds in fluorescence polarization competition
binding assay using Bocillin-FL.

| | E. coli K12 PBP4 |
|---|---|
| Ex. | Potency (EC₅₀) |
| 54 | B |
| 55 | B |

Representative results for binding to P. aeruginosa PBP3 are shown in Table 5, where A represents a potency of >500 μM, B represents a potency between 30 μM and 500 μM inclusive, C represents a potency between 10 and 30 μM, and D represents a potency <10 μM. NT=Not Tested.

TABLE 5
Binding affinity to P. aeruginosa PBP3 by Exemplary
Compounds in fluorescence polarization competition
binding assay using Bocillin-FL.

| | P. aeruginosa | | P. aeruginosa | | P. aeruginosa |
|---|---|---|---|---|---|
| Ex. | PBP3 Potency | Ex. | PBP3 Potency | Ex. | PBP3 Potency |
| 1 | D | 2 | D | 3 | D |
| 4 | D | 5 | D | 6 | D |
| 7 | D | 8 | D | 9 | D |
| 10 | D | 11 | NT | 12 | NT |
| 13 | D | 14 | D | 15 | D |
| 16 | D | 17 | D | 18 | D |
| 19 | D | 20 | D | 21 | D |
| 22 | D | 23 | D | 24 | B |
| 25 | B | 26 | D | 27 | D |
| 28 | B | 29 | D | 30 | D |
| 31 | D | 32 | A | 33 | D |
| 34 | D | 35 | D | 36 | D |
| 37 | D | 38 | D | 39 | D |
| 40 | D | 41 | D | 42 | D |
| 43 | C | 44 | C | 45 | D |
| 46 | D | 47 | D | 48 | D |
| 49 | D | 50 | D | 51 | D |
| 52 | A | 53 | D | 54 | NT |
| 55 | NT | 56 | NT | 57 | C |
| 58 | B | 59 | NT | 60 | C |
| 61 | B | 62 | B | 63 | B |
| 64 | B | 65 | B | 66 | NT |
| 67 | NT | 68 | D | 69 | C |
| 70 | D | 71 | D | 72 | D |
| 73 | D | 74 | D | 75 | D |
| 76 | D | 77 | B | 78 | D |
| 79 | D | 80 | D | 81 | B |
| 82 | D | 83 | C | 84 | D |
| 85 | C | 86 | D | 87 | D |
| 88 | C | 89 | D | 90 | D |
| 91 | D | 92 | B | 93 | D |
| 94 | NT | 95 | D | 96 | D |
| 97 | D | 98 | D | 99 | D |
| 100 | D | 101 | D | 102 | D |
| 103 | D | 104 | D | 105 | D |
| 106 | D | 107 | D | 108 | B |
| 109 | D | 110 | B | 111 | D |
| 112 | D | 113 | D | 114 | D |
| 115 | C | 116 | D | 117 | D |
| 118 | D | 119 | NT | 120 | D |
| 121 | D | 122 | D | 123 | D |
| 124 | D | 125 | A | 126 | D |
| 127 | D | 128 | C | 129 | D |
| 130 | D | 131 | D | 132 | D |
| 133 | D | 134 | D | 135 | D |
| 136 | B | 137 | D | 138 | D |
| 139 | D | 140 | D | 141 | D |
| 142 | C | 143 | B | 144 | D |
| 145 | D | 146 | D | 147 | D |
| 148 | D | 149 | C | 150 | D |
| 151 | D | 152 | D | 153 | D |
| 154 | D | 155 | D | 156 | D |
| 157 | D | 158 | C | 159 | D |
| 160 | D | 161 | D | 162 | B |
| 163 | D | 164 | D | 165 | D |
| 166 | NT | 167 | D | 168 | D |
| 169 | C | 170 | D | 171 | D |
| 172 | D | 173 | D | 174 | D |
| 175 | D | 176 | D | 177 | D |
| 178 | D | 179 | D | 180 | D |
| 181 | D | 182 | D | 183 | D |
| 184 | D | 185 | D | 186 | D |
| 187 | B | 188 | D | 189 | D |
| 190 | D | 191 | D | 192 | D |
| 193 | D | 194 | D | 195 | D |
| 196 | D | 197 | D | 198 | D |
| 199 | D | 200 | C | 201 | D |
| 202 | C | 203 | D | 204 | B |
| 205 | C | 206 | B | 207 | D |
| 208 | D | 209 | D | 210 | B |
| 211 | D | 212 | D | 213 | D |
| 215 | C | 216 | C | 217 | D |
| 218 | C | 219 | D | 220 | D |
| 221 | C | 222 | D | 223 | D |
| 224 | B | 226 | B | 230 | C |
| 231 | B | 232 | D | 233 | D |
| 234 | D | 235 | D | 236 | D |
| 237 | D | 238 | D | 239 | C |
| 240 | D | 241 | D | 242 | D |
| 243 | D | | | | |

Representative results for binding to A. baumannii PBP1a, PBP2 and PBP3 are shown in Table 6, where A represents a potency of >100 μM, B represents a potency between 30 μM and 100 μM inclusive, C represents a potency between 10 and 30 μM, and D represents a potency <10 μM. NT=Not Tested.

TABLE 6
Binding affinity to A. baumannii PBP1a, PBP2, and
PBP3 by Exemplary Compounds in fluorescence polarization
competition binding assay using Bocillin-FL.

| | A. baumannii | A. baumannii | 4. baumannii |
|---|---|---|---|
| Ex. | PBP1a Potency | PBP2 Potency | PBP1a Potency |
| 1 | C | NT | NT |
| 2 | C | NT | D |
| 3 | B | A | D |
| 5 | C | NT | D |
| 6 | NT | NT | A |
| 7 | NT | NT | A |
| 8 | NT | NT | A |
| 9 | NT | NT | B |
| 11 | NT | NT | C |
| 13 | NT | NT | D |
| 14 | NT | NT | D |
| 15 | NT | NT | A |
| 16 | NT | NT | B |
| 23 | A | A | B |
| 25 | NT | NT | A |
| 26 | A | A | B |
| 27 | A | A | B |
| 31 | NT | NT | D |
| 34 | A | B | A |
| 35 | NT | NT | B |
| 36 | NT | NT | C |
| 37 | NT | NT | B |
| 38 | NT | NT | C |
| 39 | NT | NT | B |
| 40 | A | B | A |
| 41 | NT | NT | B |
| 42 | NT | NT | B |
| 71 | NT | NT | A |
| 73 | NT | NT | D |
| 74 | B | A | D |
| 80 | NT | NT | D |
| 84 | NT | NT | B |
| 85 | NT | NT | A |
| 86 | NT | NT | C |
| 102 | A | A | B |
| 110 | NT | NT | A |
| 124 | NT | NT | D |
| 125 | NT | NT | A |
| 131 | B | B | D |
| 132 | B | A | D |
| 133 | NT | NT | D |
| 134 | A | B | D |
| 179 | A | A | A |
| 180 | A | A | A |
| 181 | A | A | A |
| 182 | A | A | A |
| 188 | NT | NT | D |
| 189 | A | A | C |
| 190 | A | A | A |
| 191 | A | B | B |
| 207 | NT | NT | C |
| 208 | NT | NT | B |
| 210 | NT | NT | A |
| 211 | NT | NT | B |
| 226 | NT | NT | A |
| 227 | NT | NT | A |
| 228 | NT | NT | C |
| 229 | NT | NT | C |

Representative results for binding to N. gonorrhoeae PBP2 are shown in Table 7, where A represents a potency of >100 μM, B represents a potency between 30 μM and 100 μM inclusive, C represents a potency between 10 and 30 μM, and D represents a potency <10 μM. NT=Not Tested.

TABLE 7
Binding affinity to N. gonorrhoeae PBP2 by
Exemplary Compounds in fluorescence polarization
competition binding assay using Bocillin-FL.

| | N. | | N. | | N. |
|---|---|---|---|---|---|
| | gonorrhoeae | | gonorrhoeae | | gonorrhoeae |
| Ex. | PBP2 Potency | Ex. | PBP2 Potency | Ex. | PBP2 Potency |
| 31 | D | 45 | D | 68 | D |
| 72 | D | 82 | D | 83 | D |
| 84 | D | 85 | C | 91 | D |
| 92 | D | 94 | D | 95 | D |
| 102 | C | 113 | D | 123 | D |
| 124 | D | 139 | D | 140 | D |
| 146 | D | 147 | D | 148 | D |
| 149 | B | 151 | D | 152 | C |
| 154 | D | 155 | D | 159 | D |
| 168 | D | 174 | D | 175 | D |
| 176 | D | 177 | C | 178 | A |
| 179 | A | 180 | A | 181 | A |
| 182 | A | 183 | D | 184 | D |
| 185 | D | 186 | D | 187 | C |
| 188 | D | 189 | C | 190 | A |
| 191 | B | 192 | C | 193 | C |
| 194 | B | 195 | C | 196 | D |
| 199 | C | 200 | A | 201 | C |
| 202 | A | 203 | D | | |

### Example II: Experimental Method to Assess Binding to Penicillin-Binding Proteins: Radioligand Competition Binding Assay

To determine the ability of boronic acid-based test PBP inhibitors to bind Penicillin Binding Proteins (PBPs), a radio-labelled boronic acid PBP inhibitor:

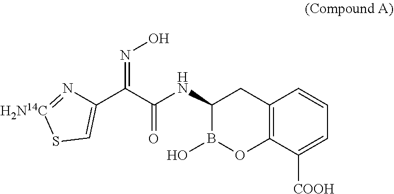

was used in competition binding assays to determine boronic acid PBP inhibitor binding to PBP1a or PBP1b from Escherichia coli. PBP1a and PBP1b were purified as described previously (Bertsche, U.; et al., J Biol. Chem. 2005, 280 (45), 38096-38101; Born, P.; et al., J Biol. Chem. 2006. 281 (37), 26985-26993). To establish assay conditions for competition binding, an enzyme titration/saturation binding experiment was performed. Saturation binding was performed in a buffer comprised of 20 mM Tris (pH 7.5), 500 mM NaCl and 0.1% (v/v) TritonX-100, with PBP1a/PBP1b at a final concentration of 0.1 μM and Compound A at final concentrations ranging from 0-100 μM. Mixtures were incubated for 60 minutes, then applied to Zeba Spin Desalting columns or plates (ThermoFisher Scientific) and centrifuged at 1000-1500×g for 2 minutes. The flow through, containing PBP bound with Compound A, was recovered and 100 μL mixed with 5 mL of UltimaGold liquid scintillation cocktail (Perkin Elmer), and the radioactivity counted using a Beckman Coulter LS 6500 Multipurpose scintillation counter. PBP binding of Compound A approached saturation at 20 μM of ¹⁴C-labeled probe. The competition binding assay was validated using the beta-lactam ampicillin, with Compound A at a final concentration of 20 μM, PBP1a/PBP1b at a final concentration of 0.1 μM. PBP1a/PBP1b was incubated with ampicillin in a 96-well microplate for 60 minutes, then Compound A was added and the mixtures incubated for an additional 60 minutes. The mixtures were then applied to Zeba Spin desalting plates and centrifuged at 1000×g for 2 minutes. The flow through was recovered and 100 μl mixed with 5 ml of UltimaGold liquid scintillation cocktail and the radioactivity counted. Ampicillin inhibited binding of Compound A with an EC₅₀ (the concentration of inhibitor required to reduce binding of Compound A by 50%) of less than 0.5 μM. Binding assays with boronic acid PBP inhibitors were performed in an identical fashion. The potency of boronic acid PBP inhibitors was reported as the EC₅₀ value.

Representative results for binding to E. coli PBP1a and PBP1b are shown in Table 6, where A represents a potency of >100 μM, B represents a potency between 10 μM and 100 μM inclusive, and C represents a potency of <10 μM. NT=Not Tested.

TABLE 6
Binding affinity to E. coli PBP1a and PBP1b
by Exemplary Compounds in competition binding assay
using ¹⁴C-labeled boronic acid probe.

| | E. coli K12 PBP1a | E. coli K12 PBP1b |
|---|---|---|
| Ex. | EC₅₀ | EC₅₀ |
| 54 | NT | B |
| 55 | NT | A |

### Example III: In Vitro Antibacterial Assays

To determine the ability of test compounds to inhibit the growth of bacterial strains, classic cell based broth microdilution minimum inhibitory concentration (MIC) assays were employed. MIC assays are performed according to CLSI methods except where otherwise noted (CLSI, 2018 and CLSI, 2019). The reference type strain E. coli ATCC 25922; the wild-type parent strain E. coli AG100; the hyper-permeable E. coli 901C and E. coli D22; and the E. coli AG100A strain lacking the acrAB efflux pump encoding genes were used to determine the ability of the PBP inhibitors to penetrate the outer membrane of gram-negative bacteria and inhibit bacterial growth. Three additional challenge isolates of Klebsiella pneumoniae (K. pneumoniae 848844 producing SHV-11 and KPC-2, K. pneumoniae UMM producing SHV-5 and KPC-2 and K. pneumoniae SI-117 producing VIM-1) were used to further assess antibacterial activity in Enterobacteriaceae and demonstrate activity of the PBP inhibitors irrespective of the beta-lactamase content of these organisms. The P. aeruginosa ATCC 27853 and A. baumannii ATCC 19606, along with the hyper-permeable P. aeruginosa ATCC 35151 and an engineered efflux pump-compromised strain of P. aeruginosa (ΔmexAB-oprM) were used to determine the ability of PBP inhibitors to penetrate the outer membrane of P. aeruginosa and A. baumannii and assess antibacterial activity against these important gram-negative organisms.

Briefly, cryo-preserved bacterial cultures of challenge strains are streaked for isolation on appropriate agar medium, in this case cation-adjusted Mueller Hinton agar. Following incubation to allow growth of the colonies, plates are sealed with parafilm and stored refrigerated for up to two weeks. For preparation of assay inoculum and to ensure low variability, at least 5 colonies are picked from the agar plates with an inoculating loop and aseptically transferred to a culture tube containing 3 mL of cation-adjusted Mueller Hinton broth (CAMHB). The broth culture is grown for 3-5 hours at 37° C. with shaking at 200 rpm. Meanwhile, 2-fold serial dilutions of test compounds are conducted in a 96-well plate with a final volume of 75 μL per well at 2-fold the final desired concentration. After the dilution plates are set up the growing cultures are then diluted in a cuvette containing CAMHB and the optical density is measured at 600 nm. The inoculum is diluted such that 75 μL of this culture in CAMHB results in a starting bacterial concentration of 2-8×10⁵ CFU/mL when added to the dilution plates. The plates are incubated for 16-20 hours for Enterobacteriaceae and Pseudomonas and 20-24 hours for Acinetobacter at 37° C. The MIC values are read visually as the lowest concentration well with no bacterial growth.

Representative results for MIC testing in Enterobacteriaceae are shown in Table 7, where A represents an MIC≥128 μg/mL, B represents an MIC of 32 to 64 μg/mL, C represents an MIC from 8 to 16 μg/mL, D represents an MIC from 2 to 4 μg/mL, E represents an MIC from 0.5 to 1 μg/mL, and F represents an MIC≤0.25 μg/mL. NT=Not Tested.

TABLE 7
Inhibition of bacterial growth. Minimum Inhibitory Concentrations
of Exemplary Compounds for Enterobacteriaceae in MHB.

| | E. coli | E. coli | E. coli | E. coli | E. coli | K. pneumoniae | K. pneumoniae | K. pneumoniae |
|---|---|---|---|---|---|---|---|---|
| Ex. | 25922 | 901C | D22 | AG100 | AG100A | 848844 | UMM | SI-117 |
| 1 | A | C | C | B | C | B | C | B |
| 2 | C | E | D | D | E | C | D | C |
| 3 | A | C | B | B | B | B | B | A |
| 11 | B | E | C | B | D | A | D | B |
| 13 | C | E | D | C | E | B | C | C |
| 14 | C | E | D | C | E | B | C | B |
| 15 | A | E | C | A | B | A | A | A |
| 16 | C | D | D | C | C | B | B | B |
| 17 | A | E | D | A | C | A | B | A |
| 18 | C | E | D | B | D | B | C | B |
| 19 | A | E | D | A | B | A | A | A |
| 20 | A | F | D | B | C | A | B | A |
| 21 | A | F | D | B | D | A | B | A |
| 22 | B | F | D | B | D | A | B | B |
| 24 | B | D | C | B | C | A | B | B |
| 29 | B | E | D | B | D | A | B | B |
| 30 | C | E | D | B | D | A | B | B |
| 31 | B | F | D | B | D | A | B | A |
| 32 | A | A | A | A | A | A | A | A |
| 33 | A | D | D | A | A | A | A | A |
| 39 | C | E | D | C | C | A | B | A |
| 43 | A | A | A | A | A | A | A | A |
| 44 | A | A | A | A | A | A | A | A |
| 45 | B | D | F | B | D | A | B | A |
| 46 | B | D | F | B | D | A | B | B |
| 47 | A | C | D | A | D | A | A | A |
| 48 | B | D | F | A | C | A | A | A |
| 49 | A | B | C | A | A | A | A | A |
| 50 | B | F | D | B | D | A | B | A |
| 51 | B | D | C | B | C | B | C | B |
| 52 | A | A | A | A | A | A | A | A |
| 53 | A | C | A | A | A | A | A | A |
| 54 | A | E | B | A | A | A | A | A |
| 55 | B | E | C | A | B | A | A | A |
| 56 | B | E | C | B | C | B | C | B |
| 57 | B | D | C | A | B | B | B | A |
| 58 | A | B | A | A | A | A | A | A |
| 59 | A | D | B | A | B | A | A | A |
| 60 | A | C | B | A | B | A | A | A |
| 61 | B | E | C | B | B | A | A | A |
| 62 | A | B | B | A | B | A | A | A |
| 63 | A | C | A | A | A | A | A | A |
| 64 | A | B | A | A | A | A | A | A |
| 65 | A | B | A | A | B | A | A | A |
| 66 | A | B | B | A | B | A | A | A |
| 67 | B | E | C | A | B | A | B | B |
| 68 | B | F | D | B | D | A | B | B |
| 69 | B | F | D | B | D | B | B | B |
| 70 | A | E | C | A | D | A | A | A |
| 72 | B | F | D | B | D | A | B | A |
| 73 | D | F | D | D | D | C | E | C |
| 74 | A | F | D | C | C | A | A | A |
| 75 | A | E | C | A | C | A | A | A |
| 76 | B | F | D | C | D | A | B | A |
| 77 | A | C | C | A | B | A | A | A |
| 78 | A | D | C | A | A | A | A | A |
| 79 | C | F | D | C | D | B | C | B |
| 81 | B | E | C | B | C | A | A | A |
| 82 | B | F | D | C | D | A | B | B |
| 83 | B | F | D | B | C | A | B | A |
| 84 | C | E | D | C | D | B | C | A |
| 85 | A | C | B | A | B | A | A | A |
| 86 | B | D | C | B | D | B | B | B |
| 87 | C | F | D | C | E | B | C | B |
| 88 | A | C | B | A | B | A | A | A |
| 89 | B | D | C | B | D | B | B | B |
| 90 | A | E | C | B | B | A | A | A |
| 91 | B | F | D | C | D | A | B | A |
| 92 | A | D | A | A | A | A | A | A |
| 93 | C | E | D | C | D | B | C | B |
| 94 | B | E | C | B | D | B | B | B |
| 95 | C | F | E | C | E | B | C | B |
| 96 | A | E | C | A | C | A | A | A |
| 97 | A | E | C | A | C | A | A | A |
| 98 | C | F | E | C | E | A | B | B |
| 99 | B | F | D | B | D | B | B | B |
| 100 | B | F | D | B | D | B | B | B |
| 101 | B | F | D | B | D | A | B | A |
| 103 | B | F | D | B | D | A | B | A |
| 104 | B | F | D | B | D | A | B | B |
| 105 | C | F | E | C | E | B | C | B |
| 106 | A | D | C | A | B | A | A | A |
| 107 | B | E | C | A | D | A | A | A |
| 108 | C | D | C | B | D | A | B | B |
| 109 | B | D | C | A | C | A | B | A |
| 110 | A | C | B | A | C | A | A | A |
| 111 | C | E | D | C | E | C | C | C |
| 112 | B | E | D | B | D | A | B | A |
| 113 | B | F | D | B | D | A | B | A |
| 114 | B | D | C | B | D | B | B | B |
| 115 | A | D | C | A | C | A | A | A |
| 116 | C | F | E | C | D | A | B | B |
| 117 | B | D | D | B | D | B | B | B |
| 118 | B | D | C | B | D | A | B | A |
| 119 | C | F | E | C | E | B | C | B |
| 120 | A | D | C | A | C | A | B | A |
| 121 | C | E | D | C | D | B | C | B |
| 122 | B | D | C | B | C | A | B | B |
| 123 | A | F | D | B | C | A | A | A |
| 124 | B | F | D | C | D | A | B | A |
| 125 | B | F | D | B | D | B | B | B |
| 126 | C | F | E | C | EE | B | C | C |
| 127 | C | D | D | C | C | B | C | B |
| 128 | B | C | C | B | B | A | B | A |
| 129 | B | E | D | B | C | B | B | B |
| 130 | B | F | D | B | C | B | C | B |
| 135 | C | F | D | B | E | B | B | B |
| 136 | A | E | C | A | C | A | A | A |
| 137 | B | F | C | B | C | A | B | A |
| 138 | B | F | D | B | C | A | B | A |
| 139 | A | F | D | B | C | A | B | A |
| 140 | B | F | D | B | D | A | B | A |
| 141 | B | F | D | B | D | A | B | A |
| 142 | A | D | C | B | B | A | B | A |
| 143 | A | E | C | A | B | A | A | A |
| 144 | B | F | D | B | C | A | B | A |
| 145 | B | F | D | B | C | A | B | A |
| 146 | B | F | D | B | D | A | B | A |
| 147 | B | F | D | B | D | A | B | A |
| 148 | A | F | D | B | C | A | B | A |
| 149 | A | E | C | A | B | A | A | A |
| 150 | A | F | D | A | B | A | A | A |
| 151 | A | F | D | B | C | A | A | A |
| 152 | A | E | C | A | B | A | A | A |
| 153 | B | E | D | B | C | A | B | A |
| 154 | B | F | D | B | C | A | B | SA |
| 155 | A | F | D | B | C | A | A | A |
| 156 | B | E | C | B | D | B | B | B |
| 157 | B | F | D | B | C | A | B | A |
| 158 | A | D | C | A | C | A | A | A |
| 159 | C | E | D | C | D | B | C | B |
| 160 | B | F | D | B | D | B | B | A |
| 161 | A | D | B | A | C | A | A | A |
| 162 | A | D | B | A | B | A | A | A |
| 163 | B | E | C | B | D | A | B | A |
| 164 | B | F | D | B | D | A | B | A |
| 165 | A | D | B | A | C | A | A | A |
| 166 | C | F | D | C | E | B | B | B |
| 167 | B | D | C | B | C | A | B | A |
| 168 | C | F | E | C | E | B | C | B |
| 169 | C | D | B | B | B | A | A | A |
| 170 | C | E | D | C | D | B | C | B |
| 171 | B | F | D | B | D | A | B | A |
| 172 | B | F | D | C | D | A | B | A |
| 173 | C | F | D | C | D | A | B | B |
| 174 | B | E | D | C | D | A | B | A |
| 175 | B | F | D | C | D | A | B | A |
| 176 | B | F | D | B | D | A | B | A |
| 177 | A | D | B | A | C | A | A | A |
| 178 | B | D | D | C | C | B | C | B |
| 181 | B | F | D | B | D | A | B | B |
| 182 | C | F | E | C | E | B | C | B |
| 183 | C | E | D | C | D | B | C | B |
| 184 | B | F | D | B | D | A | B | A |
| 185 | B | F | D | B | D | A | B | B |
| 186 | C | E | D | C | D | B | B | B |
| 187 | A | D | B | A | C | A | A | A |
| 188 | B | F | D | B | D | A | B | A |
| 189 | D | E | E | C | D | D | A | C |
| 192 | A | D | B | A | B | A | A | A |
| 193 | C | E | D | C | E | B | C | B |
| 194 | C | E | D | C | D | B | C | B |
| 195 | C | E | D | C | D | B | C | B |
| 196 | B | F | E | B | D | B | C | B |
| 197 | C | F | E | C | E | A | C | B |
| 198 | C | F | E | C | E | A | B | B |
| 199 | B | E | C | A | D | A | A | A |
| 200 | A | C | A | A | B | A | A | A |
| 201 | A | E | D | A | B | A | A | A |
| 202 | A | E | C | A | B | A | A | A |
| 203 | A | F | D | B | C | A | B | A |
| 204 | A | C | A | A | B | A | A | A |
| 205 | A | C | B | A | C | A | A | A |
| 206 | A | B | A | A | B | A | A | A |
| 209 | B | F | D | B | D | A | B | A |
| 212 | C | E | D | C | D | B | C | B |
| 213 | B | F | D | B | D | A | B | A |
| 215 | B | F | D | B | D | A | B | B |
| 216 | A | D | C | A | B | A | A | A |
| 217 | B | E | D | B | C | A | B | A |
| 218 | A | E | C | A | B | A | A | A |
| 219 | B | E | D | B | D | A | B | B |
| 220 | A | A | B | A | B | B | F | D |
| 221 | B | A | A | A | A | A | E | C |
| 222 | B | A | A | A | A | A | F | D |
| 223 | A | A | A | A | A | A | D | C |
| 224 | A | A | A | A | A | A | C | A |
| 225 | A | A | A | A | A | A | B | A |
| 230 | A | A | A | A | A | A | D | B |
| 231 | A | A | A | A | A | A | B | A |
| 232 | C | B | B | B | B | C | F | D |
| 233 | C | B | B | B | B | C | F | D |
| 234 | C | B | C | B | B | C | E | D |
| 235 | C | B | B | B | B | B | F | D |
| 236 | B | B | B | B | B | B | E | D |
| 237 | A | A | A | A | A | A | D | B |
| 238 | D | B | C | B | B | C | F | E |
| 239 | A | A | A | A | A | A | D | C |
| 240 | B | A | B | A | B | B | F | D |
| 241 | C | A | B | B | B | B | F | D |
| 242 | C | B | C | B | B | B | E | D |
| 243 | B | A | B | B | B | B | E | D |
| 244 | C | A | B | A | B | B | F | D |
| 245 | A | A | A | A | A | A | D | C |
| 246 | B | A | B | A | A | A | D | C |
| 248 | C | A | C | B | B | B | F | E |
| 249 | B | A | B | A | B | B | F | D |
| 250 | B | A | B | A | A | A | F | D |
| 251 | A | A | A | A | A | A | D | C |

Representative results for testing in P. aeruginosa and A. baumannii strains are shown in Table 8, where A represents an MIC≥128 μg/mL, B represents an MIC of 32 to 64 μg/mL, C represents an MIC from 8 to 16 μg/mL, D represents an MIC from 2 to 4 μg/mL, E represents an MIC from 0.5 to 1 μg/mL, and F represents an MIC≤0.25 μg/mL. NT=Not Tested.

TABLE 8
Inhibition of bacterial growth. Minimum Inhibitory Concentrations of Exemplary
Compounds for P. aeruginosa and A. baumannii strains in MHB.

| | P. aeruginosa | P. aeruginosa | P. aeruginosa | P. aeruginosa | A. baumannii |
|---|---|---|---|---|---|
| Ex. | PAO1 | ATCC 27853 | ATCC 35151 | ΔmexAB-OprM | ATCC 19606 |
| 13 | NT | B | F | D | A |
| 14 | NT | A | E | C | A |
| 15 | NT | B | F | C | A |
| 16 | NT | B | F | C | A |
| 17 | NT | A | D | C | A |
| 18 | NT | A | E | C | A |
| 19 | NT | A | E | B | A |
| 20 | NT | A | E | C | A |
| 21 | NT | A | D | B | A |
| 22 | NT | A | E | C | A |
| 24 | NT | A | D | B | A |
| 29 | NT | A | E | C | A |
| 30 | NT | A | E | C | A |
| 31 | NT | B | F | C | A |
| 32 | NT | A | A | A | A |
| 33 | NT | A | E | B | A |
| 39 | NT | C | F | C | A |
| 43 | NT | A | D | A | A |
| 44 | NT | A | B | A | A |
| 45 | NT | A | F | D | A |
| 46 | NT | A | F | D | A |
| 47 | NT | B | F | D | A |
| 48 | NT | A | E | C | A |
| 49 | NT | A | D | B | A |
| 50 | NT | A | E | D | A |
| 51 | NT | A | E | C | A |
| 52 | NT | A | A | A | A |
| 54 | NT | NT | C | B | A |
| 55 | A | NT | E | B | A |
| 56 | A | NT | E | B | A |
| 57 | A | NT | D | B | A |
| 58 | A | NT | C | A | A |
| 59 | A | NT | E | C | A |
| 60 | A | NT | D | B | A |
| 61 | A | NT | C | A | A |
| 62 | A | NT | C | A | A |
| 63 | A | NT | C | A | A |
| 64 | A | NT | C | A | A |
| 65 | A | NT | C | A | A |
| 66 | A | NT | C | A | A |
| 67 | A | NT | E | B | A |
| 68 | NT | A | E | D | A |
| 69 | NT | A | C | B | A |
| 70 | NT | A | C | C | A |
| 72 | NT | A | D | C | A |
| 73 | NT | D | E | E | E |
| 74 | NT | C | E | E | A |
| 75 | NT | A | C | B | A |
| 76 | NT | A | D | C | A |
| 77 | NT | A | C | A | A |
| 78 | NT | B | E | B | A |
| 79 | NT | A | E | D | A |
| 81 | NT | A | D | B | A |
| 82 | NT | A | F | B | B |
| 83 | NT | B | E | C | A |
| 84 | NT | B | E | C | A |
| 85 | NT | A | D | B | A |
| 86 | NT | A | E | C | A |
| 87 | NT | A | E | C | A |
| 88 | NT | A | D | A | A |
| 89 | NT | A | E | C | A |
| 90 | NT | A | E | A | A |
| 91 | NT | A | E | B | A |
| 92 | NT | A | C | A | A |
| 93 | NT | A | E | B | A |
| 94 | NT | A | D | B | A |
| 95 | NT | A | D | B | A |
| 96 | NT | A | D | B | A |
| 97 | NT | A | C | B | A |
| 98 | NT | A | E | C | A |
| 99 | NT | A | D | C | A |
| 100 | NT | A | D | C | A |
| 101 | NT | A | E | C | A |
| 103 | NT | A | D | C | A |
| 104 | NT | A | D | C | A |
| 106 | NT | A | D | C | A |
| 107 | NT | A | C | C | A |
| 108 | NT | A | C | B | A |
| 109 | NT | A | D | C | A |
| 110 | NT | A | C | B | A |
| 111 | NT | A | E | C | A |
| 112 | NT | A | D | C | A |
| 113 | NT | A | E | C | A |
| 114 | NT | A | E | D | A |
| 115 | NT | A | D | C | A |
| 116 | NT | A | E | C | A |
| 117 | NT | B | F | D | B |
| 118 | NT | A | E | C | A |
| 119 | NT | A | D | C | A |
| 120 | NT | A | E | C | A |
| 121 | NT | A | E | C | A |
| 122 | NT | A | D | B | A |
| 123 | NT | A | E | C | A |
| 124 | NT | B | F | D | A |
| 125 | NT | A | D | B | A |
| 126 | NT | A | D | C | A |
| 127 | NT | A | E | C | A |
| 128 | NT | A | D | B | A |
| 129 | NT | A | E | B | A |
| 130 | NT | A | F | B | A |
| 135 | NT | A | D | B | A |
| 136 | NT | A | D | A | A |
| 137 | NT | A | E | C | A |
| 138 | NT | A | E | B | A |
| 139 | NT | A | E | C | A |
| 140 | NT | A | F | C | A |
| 141 | NT | A | F | B | A |
| 142 | NT | A | D | A | A |
| 143 | NT | A | D | A | A |
| 144 | NT | A | E | C | B |
| 145 | NT | A | F | C | A |
| 146 | NT | B | F | C | B |
| 147 | NT | A | F | C | A |
| 148 | NT | A | F | C | A |
| 149 | NT | A | D | A | A |
| 150 | NT | A | E | A | A |
| 151 | NT | A | E | C | A |
| 152 | NT | A | E | B | A |
| 153 | NT | B | E | C | A |
| 154 | NT | A | F | C | B |
| 155 | NT | A | E | C | A |
| 156 | NT | A | E | C | A |
| 157 | NT | A | E | C | A |
| 158 | NT | A | E | B | A |
| 159 | NT | B | F | D | A |
| 160 | NT | A | F | D | A |
| 161 | NT | A | C | A | A |
| 162 | NT | A | C | A | A |
| 163 | NT | B | F | D | A |
| 164 | NT | A | F | D | A |
| 165 | NT | A | E | C | A |
| 166 | NT | C | F | D | A |
| 167 | NT | A | E | C | A |
| 168 | NT | B | F | D | A |
| 169 | NT | A | D | B | A |
| 170 | NT | B | F | D | A |
| 171 | NT | A | F | D | A |
| 172 | NT | A | F | D | A |
| 173 | NT | A | E | D | A |
| 174 | NT | A | E | C | A |
| 175 | NT | A | E | C | B |
| 176 | NT | A | D | B | A |
| 177 | NT | A | D | B | A |
| 178 | NT | A | E | C | A |
| 181 | NT | A | E | C | A |
| 182 | NT | A | D | C | B |
| 183 | NT | A | D | C | A |
| 184 | NT | A | E | C | A |
| 185 | NT | A | E | D | A |
| 186 | NT | B | F | C | A |
| 187 | NT | A | D | B | A |
| 188 | NT | A | E | B | A |
| 189 | NT | D | E | C | E |
| 192 | NT | A | D | B | A |
| 193 | NT | A | E | C | A |
| 194 | NT | A | E | C | B |
| 195 | NT | A | E | C | B |
| 196 | NT | B | F | D | A |
| 197 | NT | A | F | D | B |
| 198 | NT | A | E | D | A |
| 199 | NT | A | E | C | A |
| 200 | NT | A | D | A | A |
| 201 | NT | A | E | C | A |
| 202 | NT | A | D | A | A |
| 203 | NT | A | E | C | A |
| 204 | NT | A | B | A | A |
| 205 | NT | A | C | B | A |
| 206 | NT | A | B | A | A |
| 209 | NT | A | F | C | A |
| 212 | NT | A | E | C | A |
| 213 | NT | A | E | D | A |
| 215 | NT | A | C | B | A |
| 216 | NT | A | C | B | A |
| 217 | NT | A | D | C | A |
| 218 | NT | A | C | B | A |
| 219 | NT | A | D | C | A |
| 220 | NT | A | E | C | A |
| 221 | NT | A | D | B | A |
| 222 | NT | A | E | B | A |
| 223 | NT | A | D | B | A |
| 224 | NT | A | B | A | A |
| 225 | NT | A | A | A | A |
| 230 | NT | A | D | B | A |
| 231 | NT | A | C | A | A |
| 232 | NT | A | D | C | A |
| 233 | NT | A | C | A | A |
| 234 | NT | A | D | C | A |
| 235 | NT | A | E | C | A |
| 236 | NT | A | F | D | A |
| 237 | NT | A | E | C | A |
| 238 | NT | B | F | D | A |
| 239 | NT | A | D | C | A |
| 240 | NT | A | D | C | A |
| 241 | NT | A | D | D | A |
| 242 | NT | A | E | C | A |
| 243 | NT | A | D | C | A |
| 244 | NT | B | F | D | A |
| 245 | NT | A | E | C | A |
| 246 | NT | A | E | C | A |
| 247 | NT | C | F | D | A |
| 248 | NT | A | D | C | A |
| 249 | NT | A | E | C | A |
| 250 | NT | A | D | C | B |
| 251 | NT | A | D | B | A |

### Example IV: In Vitro Antibacterial Assays in Iron-Depleted Cation-Adjusted Mueller-Hinton Broth

To determine the ability of test compounds to inhibit the growth bacterial strains under conditions of iron-depletion, classic cell based broth microdilution minimum inhibitory concentration (MIC) assays were employed. MIC assays are performed according to CLSI methods except where otherwise noted (CLSI, 2018 and CLSI, 2019). The reference type strain E. coli ATCC 25922 was used to determine the ability of the PBP inhibitors to inhibit the growth of Enterobacteriaceae. Wild-type P. aeruginosa ATCC 27853, A. baumannii ATCC 17978 and A. baumannii ATCC 19606, along with the hyper-permeable P. aeruginosa ATCC 35151, an engineered efflux pump-compromised strain of P. aeruginosa (ΔmexAB-oprM), and an engineered efflux-pump compromised strain of A. baumannii IL123 (ABΔ3-ARAEcPore) were used to determine the ability of PBP inhibitors to penetrate the outer membrane of P. aeruginosa and A. baumannii and assess antibacterial activity against these important gram-negative organisms. Additionally, four challenge isolates of Pseudomonas aeruginosa (P. aeruginosa CDC-0054 producing VIM-4, OXA-50, and PAO; P. aeruginosa CDC-0090 producing KPC-5, OXA-50, and PAO; P. aeruginosa CDC-0095 producing OXA-50, and PAO), and four challenge isolates of Acinetobacter baumannii (A. baumannii CDC-0033 producing NDM-1, and OXA-94; A. baumannii CDC-0036 OXA-65, and OXA-24; A. baumannii CDC-0045 producing TEM-1D, OXA-23, and OXA-69; and A. baumannii CDC-0083 producing NDM-1, PER-7, OXA-23, and OXA-69) were used to further assess antibacterial activity in non-fermenters and demonstrate activity of the PBP inhibitors irrespective of the beta-lactamase content of these organisms.

Briefly, cryo-preserved bacterial cultures of challenge strains are streaked for isolation on appropriate agar medium, in this case cation-adjusted Mueller Hinton agar. Following incubation to allow growth of the colonies, plates are sealed with parafilm and stored refrigerated for up to two weeks. For preparation of assay inoculum and to ensure low variability, at least 5 colonies are picked from the agar plates with an inoculating loop and aseptically transferred to a culture tube containing 3 mL of iron-depleted cation-adjusted Mueller Hinton broth (IDM)—see below for IDM preparation. The broth culture is grown for 3-5 hours at 37° C. with shaking at 200 rpm. Meanwhile, 2-fold serial dilutions of test compounds are conducted in a 96-well plate with a final volume of 75 μL per well at 2-fold the final desired concentration. After the dilution plates are set up the growing cultures are then diluted in a cuvette containing IDM and the optical density is measured at 600 nm. The inoculum is diluted such that 75 μL of this culture in IDM results in a starting bacterial concentration of 2-8×10⁵ CFU/mL when added to the dilution plates. The plates are incubated for 16-20 hours for Enterobacteriaceae and Pseudomonas and 20-24 hours for Acinetobacter at 37° C. The MIC values are read visually as the lowest concentration well with no bacterial growth.

### Method for Iron-Depleted Cation-Adjusted Mueller Hinton Broth (IDM) Preparation:

Prepare cation-adjusted Mueller Hinton broth as per the manufacturer's recommendations and autoclave.
Add 100 g/L Chelex 100 resin, cover with foil and incubate with stirring for 2 hours
Remove Chelex 100 resin by filtration with 0.45 μm filter flask
Add the following back to the medium:
   CaCl₂ dihydrate: 82.5 mg/L
   MgCl₂ hexahydrate: 94.1 mg/L
   ZnSO₄: 10 μM
Adjust pH of medium to 7.3 with 5 N HCl
Sterilize using a 0.22 m filter flask

Representative results for testing compounds in iron-depleted media conditions are shown in Tables 9 and 10, where A represents an MIC≥128 μg/mL, B represents an MIC of 32 to 64 μg/mL, C represents an MIC from 8 to 16 μg/mL, D represents an MIC from 2 to 4 μg/mL, E represents an MIC from 0.5 to 1 μg/mL, and F represents an MIC 0.25 μg/mL. NT=Not Tested.

TABLE 9
Inhibition of bacterial growth. Minimum Inhibitory
Concentrations of Exemplary Compounds for P. aeruginosa
strains in iron-depleted MHB (IDM).

| | ATCC | ATCC | ΔmexAB- | CDC- | CDC- | CDC- | CDC- |
|---|---|---|---|---|---|---|---|
| Ex. | 27853 | 35151 | OprM | 0054 | 0090 | 0095 | 0110 |
| 1 | C | E | D | NT | NT | NT | NT |
| 2 | D | F | F | D | D | C | D |
| 3 | D | F | E | D | C | C | D |
| 4 | F | F | F | E | F | E | F |
| 5 | F | F | F | F | F | E | F |
| 6 | E | F | E | E | E | D | D |
| 7 | D | F | F | D | E | C | D |
| 8 | E | F | D | D | F | D | E |
| 9 | C | F | E | B | B | B | B |
| 10 | E | F | E | D | E | D | E |
| 11 | D | F | E | NT | NT | NT | NT |
| 23 | E | F | F | E | E | C | D |
| 25 | B | C | B | B | B | B | B |
| 26 | E | F | F | E | F | B | E |
| 27 | E | F | F | F | F | C | D |
| 28 | D | F | E | D | D | B | C |
| 34 | E | F | F | E | E | B | E |
| 35 | E | F | F | E | E | B | E |
| 36 | E | F | F | E | E | C | E |
| 37 | D | F | F | D | D | C | E |
| 38 | D | F | F | C | D | C | E |
| 40 | D | F | F | C | C | D | D |
| 41 | D | F | F | D | F | D | D |
| 42 | E | F | F | E | F | E | F |
| 71 | E | F | F | E | D | E | F |
| 73 | C | F | F | D | D | D | E |
| 74 | C | F | E | E | B | E | D |
| 80 | D | F | F | D | D | D | E |
| 102 | D | E | E | B | D | D | E |
| 125 | A | A | A | A | A | A | A |
| 131 | C | F | F | A | D | C | D |
| 132 | C | E | F | A | D | A | D |
| 133 | E | F | F | E | E | D | E |
| 134 | D | F | F | B | D | B | D |
| 179 | E | F | F | E | D | D | E |
| 180 | E | F | F | E | D | D | F |
| 189 | E | F | F | E | E | D | E |
| 190 | D | E | F | C | E | C | D |
| 191 | D | F | F | D | C | C | D |
| 207 | D | F | D | D | D | C | D |
| 208 | D | F | D | B | D | B | C |
| 210 | A | D | C | A | A | A | A |
| 211 | C | F | F | C | C | C | D |
| 226 | E | F | D | D | NT | D | E |
| 227 | E | F | F | E | NT | D | F |
| 228 | E | F | F | D | NT | NT | E |
| 229 | E | F | F | D | NT | NT | E |

TABLE 10
Inhibition of bacterial growth. Minimum Inhibitory
Concentrations of Exemplary Compounds for A. baumannii
strains in iron-depleted MHB (IDM).

| | ATCC | ATCC | IL- | CDC- | CDC- | CDC- | CDC- |
|---|---|---|---|---|---|---|---|
| Ex. | 17978 | 19606 | 2123 | 0033 | 0036 | 0045 | 0083 |
| 1 | NT | D | E | NT | NT | NT | NT |
| 2 | E | E | NT | C | B | D | C |
| 3 | C | B | NT | B | B | B | B |
| 4 | F | E | B | B | B | D | B |
| 5 | F | E | D | D | B | D | B |
| 6 | C | B | B | B | B | B | B |
| 7 | D | B | B | B | B | B | B |
| 8 | D | B | B | B | B | B | B |
| 9 | B | B | B | B | B | B | B |
| 10 | B | B | B | B | B | B | B |
| 11 | NT | A | NT | NT | NT | NT | NT |
| 23 | F | F | NT | C | C | E | E |
| 25 | B | B | NT | B | B | B | B |
| 26 | F | E | NT | D | C | E | F |
| 27 | F | F | NT | E | D | E | F |
| 28 | E | C | B | B | B | B | B |
| 34 | F | F | D | E | E | F | E |
| 35 | F | F | D | E | E | F | F |
| 36 | F | F | E | E | E | F | F |
| 37 | F | F | D | D | D | E | E |
| 38 | F | F | D | D | D | E | E |
| 40 | F | E | C | D | D | D | E |
| 41 | F | E | D | D | D | E | E |
| 42 | F | F | E | E | E | F | F |
| 71 | B | B | NT | B | B | B | B |
| 73 | D | D | NT | B | B | B | B |
| 74 | B | B | NT | B | B | B | B |
| 80 | E | D | NT | C | C | C | B |
| 102 | D | D | NT | B | B | B | B |
| 125 | A | A | NT | A | A | A | A |
| 131 | E | A | NT | A | A | A | A |
| 132 | A | C | NT | A | A | A | A |
| 133 | F | F | NT | C | D | C | A |
| 134 | E | E | NT | B | C | B | A |
| 179 | E | E | NT | C | D | C | A |
| 180 | E | E | NT | C | C | D | A |
| 189 | F | E | NT | D | D | D | B |
| 190 | C | A | NT | A | A | A | A |
| 191 | D | C | NT | A | B | A | A |
| 207 | E | E | NT | B | D | A | A |
| 208 | E | D | NT | B | C | A | A |
| 210 | A | A | NT | A | A | A | A |
| 211 | D | D | NT | B | C | A | A |
| 226 | E | E | NT | C | C | C | D |
| 227 | F | F | NT | C | D | D | D |
| 228 | E | F | NT | D | D | D | D |
| 229 | F | E | NT | D | D | C | D |

### Example V: In Vitro Antibacterial Assays for N. gonorrhoeae Strains

Additional antibacterial testing of the series was performed in 8 reference strains of N. gonorrhoeae (ATCC 49226, FA1090, WHO G, WHO L, WHO K, H041, WHO Z, and WHO Q). ATCC 49226, FA1090, WHO G, and WHO L produce wild type or wild-type like PBP2. WHO K, H041, WHO Z, and WHO Q produce mosaic PBP2. Liquid broth-based assays were used for antibacterial testing of PBP inhibitors in Neisseria gonorrhoeae. Briefly, cryo-preserved bacterial cultures of clinical strains were streaked for isolation on Chocolate Agar (72 g/L (2×) GC Agar Base (BD #228950) and 2% (2×) Hemoglobin was autoclaved at 121° C. for 20 minutes to sterilize. Once cooled to ˜50° C. the 2× GC Agar Base and 2× Hemoglobin solutions are combined and 1% IsoVitaleX Enrichment (BD #211876) was added to the solution). Strains were incubated at 36° C. and 5% CO₂ to allow growth of colonies, 24 hours before inoculum preparation. 2-fold serial dilutions of test compounds were conducted in a 96 well plate with a final volume of 75 μL per well at 2-fold the final desired concentration in Fastidious broth (Remel #R07664). For preparation of assay inoculum, a direct suspension was prepared by aseptically swabbing all colonies from agar plates into culture tubes containing 2 mL of fresh sterile saline. After the dilution plates were set up, direct suspensions were then diluted in a cuvette containing sterile saline and the optical density was measured at 600 nm. Inocula were diluted such that 75 μL of this culture in Fastidious broth results in a starting bacterial concentration of 5×10⁵ CFU/mL when added to the dilution plates. The plates were incubated for ˜24 hours at 36° C. and 5% CO₂. The MIC was read visually as the lowest concentration well with <10% bacterial growth compared to wells of full growth.

Representative results for testing in N. gonorrhoeae strains are shown in Table 11, where A represents an MIC≥64 μg/mL, B represents an MIC of 16 to 32 μg/mL, C represents an MIC from 4 to 8 μg/mL, D represents an MIC from 1 to 2 μg/mL, E represents an MIC from 0.25 to 0.5 μg/mL, and F represents an MIC≤0.125 μg/mL. NT=Not Tested.

TABLE 11
Inhibition of bacterial growth. Minimum Inhibitory Concentrations
of Exemplary Compounds for N. gonorrhoeae strains.

| | ATCC | | WHO | WHO | WHO | | WHO | WHO |
|---|---|---|---|---|---|---|---|---|
| Ex. | 49226 | FA1090 | G | L | K | H041 | Z | Q |
| 2 | A | C | A | A | A | A | NT | NT |
| 4 | B | C | C | A | A | A | NT | NT |
| 5 | A | C | B | A | A | A | NT | NT |
| 6 | A | B | B | A | A | A | NT | NT |
| 7 | A | B | A | A | A | A | NT | NT |
| 8 | A | C | B | A | A | A | NT | NT |
| 9 | A | B | A | A | A | A | NT | NT |
| 10 | A | B | A | A | A | A | NT | NT |
| 11 | B | D | C | A | A | A | B | NT |
| 12 | A | A | A | A | A | A | A | NT |
| 15 | C | E | C | A | C | C | D | NT |
| 17 | A | D | A | A | A | A | NT | NT |
| 18 | C | D | C | B | B | B | C | NT |
| 19 | B | D | C | A | A | A | NT | NT |
| 20 | C | D | C | A | B | B | NT | NT |
| 21 | A | C | A | A | A | A | NT | NT |
| 22 | A | D | B | A | A | A | NT | NT |
| 23 | A | C | B | A | A | A | NT | NT |
| 24 | A | B | B | A | A | A | NT | NT |
| 25 | A | A | A | A | A | A | NT | NT |
| 26 | A | B | A | A | A | A | NT | NT |
| 27 | A | B | B | A | A | A | NT | NT |
| 28 | A | A | A | A | A | A | NT | NT |
| 29 | A | C | B | A | A | A | NT | NT |
| 30 | A | D | B | A | A | A | NT | NT |
| 31 | D | F | D | C | C | C | D | NT |
| 32 | A | A | A | A | A | A | NT | NT |
| 33 | B | C | B | A | A | A | NT | NT |
| 34 | B | C | B | A | A | A | NT | NT |
| 35 | B | C | B | A | A | A | NT | NT |
| 36 | B | C | B | A | B | B | NT | NT |
| 37 | B | C | B | A | B | B | NT | NT |
| 38 | B | C | B | A | A | A | NT | NT |
| 39 | C | D | C | B | A | A | NT | NT |
| 40 | B | C | B | A | A | A | NT | NT |
| 41 | B | C | B | A | A | A | NT | NT |
| 42 | B | C | B | A | B | B | NT | NT |
| 43 | A | A | A | A | A | A | NT | NT |
| 44 | A | A | A | A | A | A | NT | NT |
| 45 | D | F | E | B | B | B | B | B |
| 46 | D | F | E | B | B | B | B | B |
| 47 | C | E | C | A | A | A | A | A |
| 48 | C | E | D | B | C | B | D | C |
| 49 | B | C | B | B | A | A | NT | NT |
| 50 | D | F | D | B | B | B | NT | NT |
| 51 | C | C | C | A | A | A | NT | NT |
| 52 | A | A | A | A | A | A | NT | NT |
| 53 | A | C | A | A | A | A | A | A |
| 57 | B | C | B | A | A | A | A | A |
| 58 | A | A | A | A | A | A | A | A |
| 59 | A | C | A | A | A | A | A | A |
| 60 | A | B | A | A | A | A | A | A |
| 68 | C | E | C | B | B | B | B | A |
| 69 | C | E | C | B | A | A | B | A |
| 70 | B | D | B | A | A | A | A | A |
| 71 | A | A | A | A | A | A | A | A |
| 72 | C | E | D | A | A | A | B | B |
| 73 | A | D | B | A | A | A | A | A |
| 74 | A | D | B | A | A | A | A | A |
| 75 | C | E | C | A | A | A | B | B |
| 76 | A | D | B | A | A | A | A | A |
| 77 | B | C | B | A | A | A | A | A |
| 78 | B | C | C | A | A | A | B | A |
| 79 | D | F | D | B | B | B | C | C |
| 80 | B | C | B | A | A | A | A | A |
| 81 | C | E | E | B | B | B | B | B |
| 82 | C | F | E | B | C | C | C | C |
| 83 | B | D | C | A | A | A | A | A |
| 84 | C | D | D | B | B | A | B | B |
| 85 | A | C | A | A | A | A | A | A |
| 86 | A | D | C | A | A | A | A | A |
| 87 | B | E | C | B | A | A | A | A |
| 88 | A | C | B | A | A | A | A | A |
| 89 | B | D | C | A | A | A | A | A |
| 90 | A | C | B | A | A | A | A | A |
| 91 | B | E | D | A | B | B | B | B |
| 92 | A | B | A | A | A | A | A | A |
| 93 | A | D | C | A | A | A | A | A |
| 94 | A | C | B | A | A | A | A | A |
| 95 | C | F | D | B | B | B | B | B |
| 96 | A | C | B | A | A | A | A | A |
| 97 | A | D | C | A | A | A | A | A |
| 98 | B | E | D | A | B | B | B | B |
| 99 | B | E | D | A | B | B | B | B |
| 100 | B | E | D | A | B | B | B | B |
| 101 | C | E | D | B | B | B | B | B |
| 102 | A | B | B | A | A | A | A | A |
| 103 | B | E | D | A | B | B | B | B |
| 104 | C | E | D | B | B | B | C | C |
| 105 | C | F | E | B | B | B | C | C |
| 106 | A | B | B | A | A | A | A | A |
| 107 | A | D | B | A | A | A | A | A |
| 108 | B | D | C | A | A | A | A | A |
| 109 | A | C | A | A | A | A | A | A |
| 110 | A | B | A | A | A | A | A | A |
| 111 | B | D | C | A | A | A | A | A |
| 112 | C | D | C | A | A | A | B | B |
| 113 | C | E | C | A | A | A | B | B |
| 114 | A | C | B | A | A | A | A | A |
| 115 | A | C | A | A | A | A | A | A |
| 116 | B | E | C | A | B | A | B | B |
| 117 | A | C | B | A | A | A | A | A |
| 118 | A | C | A | A | A | A | A | A |
| 119 | B | E | D | B | B | A | C | B |
| 120 | A | C | A | A | A | A | A | A |
| 121 | B | D | B | A | A | A | A | A |
| 122 | A | C | A | A | A | A | A | A |
| 123 | C | E | D | B | C | B | D | C |
| 124 | D | F | E | C | C | C | D | D |
| 125 | A | A | A | A | A | A | A | A |
| 126 | C | F | D | B | B | B | C | C |
| 127 | B | C | B | A | A | A | A | A |
| 128 | A | B | A | A | A | A | A | A |
| 129 | B | D | C | A | B | B | C | B |
| 130 | C | D | C | A | B | B | C | B |
| 131 | A | B | A | A | A | A | A | A |
| 132 | A | B | A | A | A | A | A | A |
| 133 | A | B | B | A | A | A | A | A |
| 134 | A | B | A | A | A | A | A | A |
| 135 | C | F | D | B | A | A | B | B |
| 136 | A | D | B | A | A | A | A | A |
| 137 | D | E | D | B | C | B | C | C |
| 138 | C | F | D | B | B | B | C | C |
| 139 | D | F | D | C | C | C | D | C |
| 140 | E | F | E | D | D | D | E | D |
| 141 | D | F | E | C | D | C | D | D |
| 142 | B | C | C | A | A | A | A | A |
| 143 | B | D | C | A | A | A | B | A |
| 144 | D | F | E | C | B | B | C | C |
| 145 | D | F | D | B | C | C | C | C |
| 146 | D | F | E | C | D | C | D | D |
| 147 | D | F | E | C | E | D | E | E |
| 148 | C | E | D | A | C | B | C | C |
| 149 | A | C | B | A | A | A | B | A |
| 150 | A | C | B | A | A | A | A | A |
| 151 | C | E | D | B | C | C | D | C |
| 152 | B | D | C | A | B | B | C | B |
| 153 | B | D | C | A | B | A | B | B |
| 154 | D | F | E | C | D | D | E | D |
| 155 | D | F | E | B | D | C | D | D |
| 156 | B | D | C | A | A | A | A | A |
| 157 | C | F | D | C | C | B | C | C |
| 158 | A | C | B | A | A | A | A | A |
| 159 | B | D | C | A | A | A | A | A |
| 160 | C | F | D | B | B | B | C | C |
| 161 | A | D | B | A | A | A | A | A |
| 162 | A | C | B | A | A | A | A | A |
| 163 | A | D | B | A | A | A | A | A |
| 164 | C | E | D | B | B | B | C | B |
| 165 | A | C | A | A | A | A | A | A |
| 166 | B | E | C | A | A | A | B | B |
| 167 | B | D | B | A | A | A | A | A |
| 168 | C | E | D | B | B | A | B | B |
| 169 | A | C | A | A | A | A | A | A |
| 170 | C | E | C | A | A | A | A | A |
| 171 | C | E | C | A | B | B | C | B |
| 172 | D | F | E | C | C | B | D | C |
| 173 | D | F | D | B | B | B | C | B |
| 174 | C | E | D | B | A | A | B | B |
| 175 | D | F | E | C | D | C | D | D |
| 176 | C | E | C | A | A | A | B | A |
| 177 | A | C | B | A | A | A | A | A |
| 178 | B | C | B | A | A | A | A | A |
| 179 | A | B | B | A | A | A | A | A |
| 180 | B | C | B | A | A | A | A | A |
| 181 | C | E | D | B | B | A | B | B |
| 182 | C | F | D | B | B | A | B | B |
| 183 | B | D | B | A | A | A | A | A |
| 184 | C | F | D | B | B | B | C | C |
| 185 | C | F | C | B | B | B | C | B |
| 186 | B | E | C | A | A | A | A | A |
| 187 | A | C | A | A | A | A | A | A |
| 188 | C | E | C | A | A | A | B | B |
| 189 | B | C | B | A | B | A | B | B |
| 190 | A | B | A | A | A | A | A | A |
| 191 | A | B | A | A | A | A | A | A |
| 192 | A | C | A | A | A | A | A | A |
| 193 | A | D | B | A | A | A | A | A |
| 194 | B | D | C | A | A | A | A | A |
| 195 | B | D | C | A | A | A | A | A |
| 196 | C | E | D | B | C | B | C | C |
| 197 | B | E | C | A | B | B | B | B |
| 198 | B | E | C | A | B | B | B | B |
| 199 | B | D | B | A | A | A | A | A |
| 200 | A | C | A | A | A | A | A | A |
| 201 | C | D | C | A | B | A | B | B |
| 202 | B | C | B | A | A | A | B | A |
| 203 | C | E | C | A | B | B | C | C |
| 204 | A | B | A | A | A | A | A | A |
| 205 | A | B | A | A | A | A | A | A |
| 206 | A | A | A | A | A | A | A | A |
| 207 | B | C | B | A | A | A | B | A |
| 208 | A | C | B | A | A | A | A | A |
| 209 | C | E | C | B | B | B | C | B |
| 210 | A | A | A | A | A | A | A | A |
| 211 | B | C | B | A | B | B | B | B |
| 212 | B | D | B | A | A | A | A | A |
| 213 | C | F | D | A | A | A | B | A |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound of Formula (IIa) or (IIb), or a pharmaceutically acceptable salt, solvate, stereoisomer, tautomer, N-oxide, dimer, or trimer thereof: 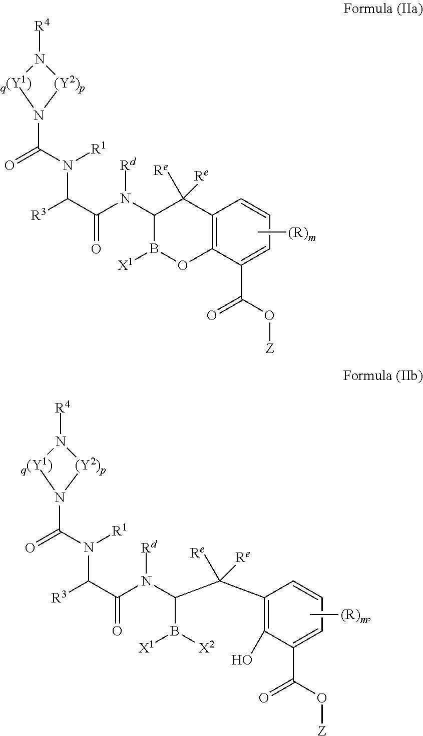
wherein:
R¹ is hydrogen or C₁-C₆ alkyl;
R³ is 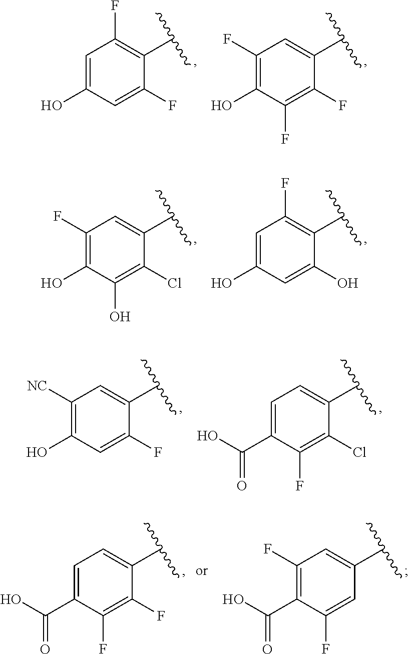
each Y¹ and Y² is independently —C(═O)— or —C(R⁵)₂—;
R⁴ is —S(═O)₂R^{b}, —S(═O)₂NR^{c}R^{d}, or —C(═O)R^{b}, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R⁵ is independently hydrogen, halogen, —OH, —CN, NH₂, NO₂, optionally substituted alkyl, optionally substituted alkoxy, optionally substituted cycloalkyl, optionally substituted heterocycloalkyl, optionally substituted aryl, or optionally substituted heteroaryl;
q is 1-3;
p is 1-3;
each R is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, optionally substituted heteroaryl, halogen, —CN, —OR^{a}, —SR^{a}, —NR^{c}R^{d}, —NR^{c}C(═O)R^{b}, —C(═O)NR^{c}R^{d}, —C(═O)R^{a}, or —C(═O)OR^{a};
m is 0-3;
R^{d} is hydrogen or C₁-C₆ alkyl;
each R^{e} is independently hydrogen, —CN, —OH, C₁-C₆ alkyl, or cycloalkyl;
X¹ and X² are independently —OH, —OR^{X}, or F; or
X¹ and X² are taken together with the boron atom to which there are attached to form an optionally substituted cyclic boronate ester;
R^{X} is C₁-C₆ alkyl or cycloalkyl;
Z is hydrogen, R⁶¹, —(R⁶⁰)_{q}OR⁶¹, (R⁶⁰)_{q}O(R⁶⁰)_{q}OR⁶¹, —R⁶⁰OC(═O)R⁶¹, —R⁶⁰OC(═O)OR⁶¹, —R⁶⁰OC(═O)NHR⁶¹, —R⁶⁰OC(═O)N(R⁶¹)₂, optionally substituted alkyloxyalkyl, optionally substituted acyloxyalkyl, optionally substituted alkyloxycarbonyloxyalkyl, optionally substituted cycloalkyloxycarbonyloxyalkyl, optionally substituted aryloxycarbonyloxyalkyl, or optionally substituted alkyl-[1,3]dioxol-2-one;
each R⁶⁰ is independently —CH₂—, —CH(CH₃)—, —C(CH₃)₂—, or 1,1′-cyclopropylene;
each R⁶¹ is independently optionally substituted C₁-C₆ alkyl, optionally substituted cycloalkyl, optionally substituted heterocycloalkyl, optionally substituted aryl, or optionally substituted heteroaryl; or
two R⁶¹ are taken together with the nitrogen to which they are attached to form an optionally substituted heterocycloalkyl;
each R^{a} is independently hydrogen, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{b} is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{c} and R^{d} are independently hydrogen, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl; or
each R^{c} and R^{d} are taken together with the nitrogen to which they are attached to form an optionally substituted heterocycloalkyl.

2. The compound of claim 2, wherein R¹ is hydrogen.

3. The compound of claim 1 or 2, wherein R³ is

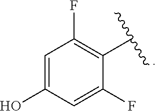 [SMILES: *c1c(F)cc(O)cc1F]

4. The compound of claim 1 or 2, wherein R³ is

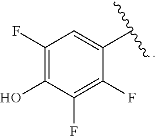 [SMILES: *c1cc(F)c(O)c(F)c1F]

5. The compound of claim 1 or 2, wherein R³ is

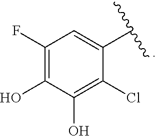 [SMILES: *c1cc(F)c(O)c(O)c1Cl]

6. The compound of claim 1 or 2, wherein R³ is

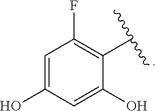 [SMILES: *c1c(O)cc(O)cc1F]

7. The compound of claim 1 or 2, wherein R³ is

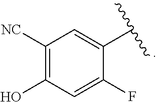 [SMILES: *c1cc(C#N)c(O)cc1F]

8. The compound of claim 1 or 2, wherein R³ is

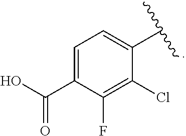 [SMILES: *c1ccc(C(=O)O)c(F)c1Cl]

9. The compound of claim 1 or 2, wherein R³ is

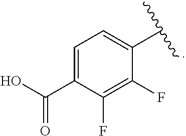 [SMILES: *c1ccc(C(=O)O)c(F)c1F]

10. The compound of claim 1 or 2, wherein R³ is

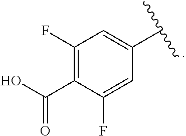 [SMILES: *c1cc(F)c(C(=O)O)c(F)c1]

11. The compound of any one of claims 1-10, wherein q is 2; each Y¹ is —C(R⁵)₂—; p is 2; and each Y² is —C(═O)—.

12. The compound of any one of claims 1-10, wherein q is 2; each Y¹ is —C(R⁵)₂—; p is 1; and Y² is —C(═O)—.

13. The compound of any one of claims 1-10, wherein q is 2; each Y¹ is —C(R⁵)₂—; p is 2; and one Y² is —C(R⁵)₂— and one Y² is —C(═O)—.

14. The compound of any one of claims 1-10, wherein q is 2; one Y¹ is —C(R⁵)₂— and one Y¹ is —C(═O)—; p is 2; and one Y² is —C(R⁵)₂— and one Y² is —C(═O)—.

15. The compound of any one of claims 1-14, wherein each R⁵ is independently hydrogen, halogen, or optionally substituted alkyl.

16. The compound of any one of claims 1-14, wherein each R⁵ is hydrogen.

17. The compound of any one of claims 1-16, wherein is

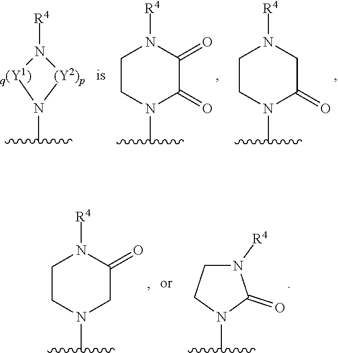

18. The compound of any one of claims 1-17, wherein

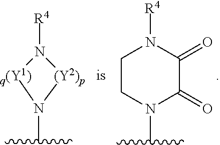

19. The compound of any one of claims 1-18, wherein

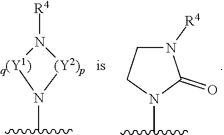

20. The compound of any one of claims 1-19, wherein R⁴ is —S(═O)₂R^{b}, optionally substituted C₁-C₆ alkyl, optionally substituted C₁-C₆ haloalkyl, optionally substituted C₁-C₆ hydroxyalkyl, or optionally substituted C₁-C₆ aminoalkyl.

21. The compound of any one of claims 1-20, wherein R⁴ is optionally substituted C₁-C₆ alkyl or optionally substituted C₁-C₆ aminoalkyl.

22. The compound of any one of claims 1-21, wherein R⁴ is C₁-C₆ alkyl or C₁-C₆ aminoalkyl.

23. The compound of any one of claims 1-22, wherein R⁴ is C₁-C₆ alkyl.

24. The compound of any one of claims 1-22, wherein R⁴ is C₁-C₆ aminoalkyl.

25. The compound of any one of claims 1-20, wherein R⁴ is —S(═O)₂R^{b}.

26. The compound of any one of claims 1-25, wherein R^{d} is hydrogen.

27. The compound of any one of claims 1-26, wherein each R^{e} is hydrogen.

28. The compound of any one of claims 1-27, wherein X¹ and X² are —OH.

29. The compound of any one of claims 1-28, wherein each R is independently optionally substituted C₁-C₆ alkyl, optionally substituted C₁-C₆ haloalkyl, optionally substituted C₁-C₆ hydroxyalkyl, halogen, —CN, —OR^{a}, —SR^{a}, —NR^{c}R^{d}, —NR^{c}C(═O)R^{b}, —C(═O)NR^{c}R^{d}, —C(═O)R^{a}, or —C(═O)OR^{a}.

30. The compound of any one of claims 1-29, wherein m is 0 or 1.

31. The compound of any one of claims 1-29, wherein m is 1 or 2.

32. The compound of any one of claims 1-31, wherein Z is hydrogen.

33. The compound of any one of claims 1-31, wherein Z is R⁶¹; and R⁶¹ is optionally substituted alkyl.

34. The compound of any one of claims 1-31, wherein Z is —R⁶⁰OC(═O)R⁶¹ or —R⁶⁰OC(═O)OR⁶¹; R⁶⁰ is —CH₂— or —CH(CH₃)—; and R⁶¹ is optionally substituted alkyl, optionally substituted cycloalkyl, or optionally substituted heterocycloalkyl.

35. A compound of Formula (Ia) or (Ib), or a pharmaceutically acceptable salt, solvate, stereoisomer, tautomer, N-oxide, dimer, or trimer thereof: 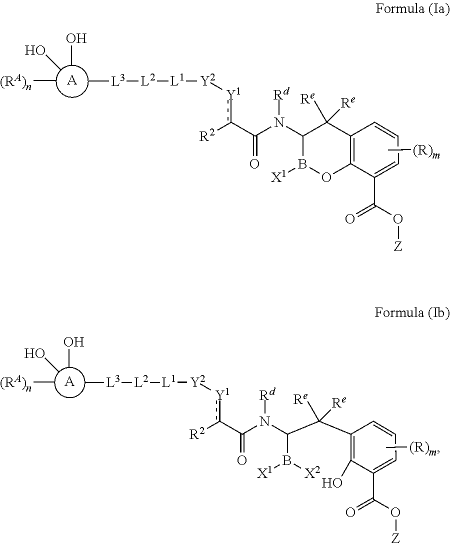
wherein: 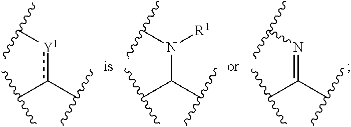
R¹ is hydrogen or C₁-C₆ alkyl;
Y² is —(C═O)— or —O—;
L¹ is absent, cycloalkyl, heterocycloalkyl, aryl, or heteroaryl; wherein the cycloalkyl, heterocycloalkyl, aryl, and heteroaryl is optionally substituted with one to six R^{L1};
each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or
two R^{L1} on the same carbon are taken together to form an oxo;
L² is absent or C₁-C₆ alkylene optionally substituted with one, two, or three R^{L2};
each R^{L2} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a};
L³ is absent, —C(═O)NH—, —NHC(═O)—, or —NH—;
Ring A is cycloalkyl, heterocycloalkyl, aryl, or heteroaryl;
each R^{A} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a};
n is 0-3;
R² is hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl is optionally substituted with one, two, or three R^{2′};
each R^{2′} is independently C₁-C₆ alkyl, C₁-C₆ haloalkyl, C₁-C₆ hydroxyalkyl, C₁-C₆ aminoalkyl, halogen, —CN, —OR^{a}, —NR^{c}R^{d}, —NR^{c}C(═O)R^{b}, —C(═O)NR^{c}R^{d}, —C(═O)R^{b}, —C(═O)OR^{a}, cycloalkyl, heterocycloalkyl, aryl, or heteroaryl;
each R is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, optionally substituted heteroaryl, halogen, —CN, —OR^{a}, —SR^{a}, —NR^{c}R^{d}, —NR^{c}C(═O)R^{b}, —C(═O)NR^{c}R^{d}, —C(═O)R^{a}, or —C(═O)OR^{a};
m is 0-3;
R^{d} is hydrogen or C₁-C₆ alkyl;
each R^{e} is independently hydrogen, —CN, —OH, C₁-C₆ alkyl, or cycloalkyl;
X¹ and X² are independently —OH, —OR^{X}, or F; or
X¹ and X² are taken together with the boron atom to which there are attached to form an optionally substituted cyclic boronate ester;
R^{X} is C₁-C₆ alkyl or cycloalkyl;
Z is hydrogen, R⁶¹, —(R⁶⁰)_{q}OR⁶¹, —(R⁶⁰)_{q}O(R⁶⁰)_{q}OR⁶¹, —R⁶⁰OC(═O)R⁶¹, —R⁶⁰OC(═O)OR⁶¹, —R⁶⁰OC(═O)NHR⁶¹, —R⁶⁰OC(═O)N(R⁶¹)₂, optionally substituted alkyloxyalkyl, optionally substituted acyloxyalkyl, optionally substituted alkyloxycarbonyloxyalkyl, optionally substituted cycloalkyloxycarbonyloxyalkyl, optionally substituted aryloxycarbonyloxyalkyl, or optionally substituted alkyl-[1,3]dioxol-2-one;
each R⁶⁰ is independently —CH₂—, —CH(CH₃)—, —C(CH₃)₂—, or 1,1′-cyclopropylene;
each R⁶¹ is independently optionally substituted C₁-C₆ alkyl, optionally substituted cycloalkyl, optionally substituted heterocycloalkyl, optionally substituted aryl, or optionally substituted heteroaryl; or
two R⁶¹ are taken together with the nitrogen to which they are attached to form an optionally substituted heterocycloalkyl;
each R^{a} is independently hydrogen, optionally substituted C₁-C₅ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{b} is independently optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl;
each R^{c} and R^{d} are independently hydrogen, optionally substituted C₁-C₆ alkyl, optionally substituted C₁-C₆ haloalkyl, optionally substituted C₁-C₆ hydroxyalkyl, optionally substituted C₁-C₆ aminoalkyl, optionally substituted cycloalkyl, optionally substituted heterocycloalkyl, optionally substituted aryl, or optionally substituted heteroaryl; or
each R^{c} and R^{d} are taken together with the nitrogen to which they are attached to form an optionally substituted heterocycloalkyl.

36. The compound of claim 35, wherein 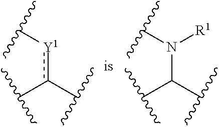
and Y² is —(C═O)—.

37. The compound of claim 36, wherein R¹ is hydrogen.

38. The compound of claim 35, wherein 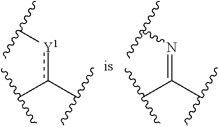
and Y² is —O—.

39. The compound of any one of claims 35-38, wherein L¹ is absent.

40. The compound of any one of claims 35-38, wherein L¹ is heterocycloalkyl optionally substituted with one to six R^{L1}; and each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or two R^{L1} on the same carbon are taken together to form an oxo.

41. The compound of any one of claims 35-38 or 40, wherein L¹ is pyrrolidine, piperidine, or piperazine; each optionally substituted with one to six R^{L1}; and each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or two R^{L1} on the same carbon are taken together to form an oxo.

42. The compound of any one of claims 35-38 or 40 or 41, wherein L¹ is piperazine optionally substituted with one to six R^{L1}; and each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or two R^{L1} on the same carbon are taken together to form an oxo.

43. The compound of any one of claims 35-38 or 40-42, wherein L¹ is 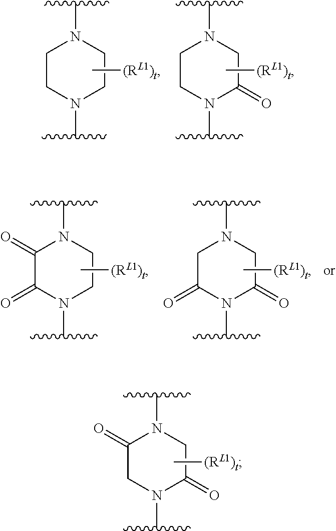
wherein each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; and t is 0-2.

44. The compound of any one of claims 35-38 or 40-43, wherein L¹ is 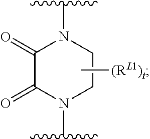
wherein each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; and t is 0-2.

45. The compound of any one of claims 35-44, wherein L² is absent.

46. The compound of any one of claims 35-44, wherein L² is C₁-C₆ alkylene optionally substituted with one, two, or three R^{L2}; and each R^{L2} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}.

47. The compound of any one of claims 35-44 or 46, wherein L² is C₂-C₄ alkylene.

48. The compound of any one of claims 35-44 or 46 or 47, wherein L² is C₂-C₃ alkylene.

49. The compound of any one of claims 35-48, wherein L³ is absent.

50. The compound of any one of claims 35-48, wherein L³ is —C(═O)NH— or —NHC(═O)—.

51. The compound of any one of claims 35-48, wherein L³ is —NH—.

52. The compound of any one of claims 35-38, wherein:
(a) L¹ is heterocycloalkyl optionally substituted with one to six R^{L1}; each R^{L1} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; or two R^{L1} on the same carbon are taken together to form an oxo; L² is C₂-C₄ alkylene; and L³ is —C(═O)NH— or —NHC(═O)—; or
(b) L¹ is absent; L² is C₁-C₆ alkylene optionally substituted with one, two, or three R^{L2};
each R^{L2} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}; and L³ is —C(═O)NH— or —NHC(═O)—; or
(c) L¹, L², and L³ are absent; or
(d) L¹ and L² are absent and L³ is —NH—.

53. The compound of any one of claims 35-38, wherein the compound of Formula (Ia) or (Ib), or a pharmaceutically acceptable salt, solvate, stereoisomer, tautomer, N-oxide, dimer, or trimer thereof; is of Formula (Ia′) or (Ib′):

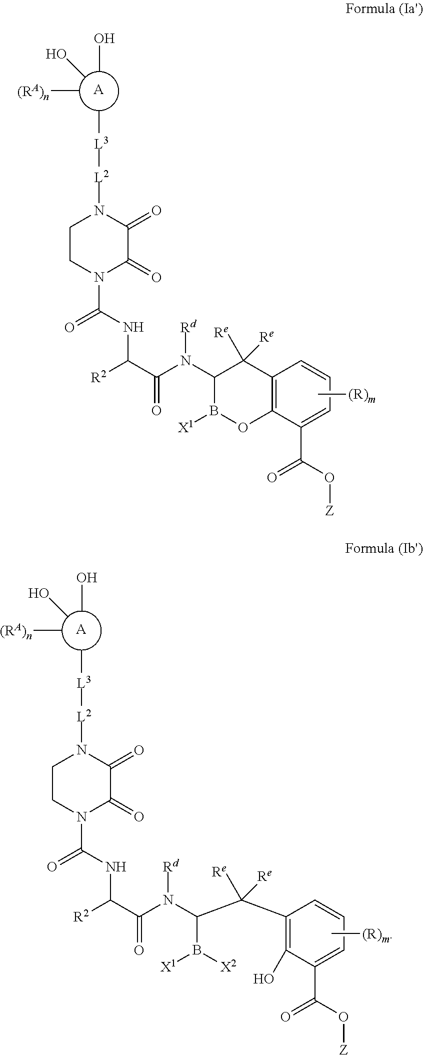

54. The compound of claim 53, wherein L² is C₁-C₆ alkylene optionally substituted with one, two, or three R^{L2}; and each R^{L2} is independently halogen, C₁-C₆ alkyl, cycloalkyl, heterocycloalkyl, —CN, —OR^{a}, —NR^{c}R^{d}, —NR^{c}C(═O)R^{b}, —C(═O)NR^{c}R^{d}, —C(═O)R^{b}, or —C(═O)OR^{a}.

55. The compound of claim 53 or 54, wherein L² is C₂-C₄ alkylene.

56. The compound of any one of claims 53-55, wherein L² is C₂-C₃ alkylene.

57. The compound of any one of claims 53-56, wherein L³ is —C(═O)NH— or —NHC(═O)—.

58. The compound of any one of claims 35-57, wherein R^{d} is hydrogen.

59. The compound of any one of claims 35-58, wherein each R^{e} is hydrogen.

60. The compound of any one of claims 35-59, wherein X¹ and X² are —OH.

61. The compound of any one of claims 35-60, wherein each R is independently optionally substituted C₁-C₆ alkyl, optionally substituted C₁-C₆ haloalkyl, optionally substituted C₁-C₆ hydroxyalkyl, halogen, —CN, —OR^{a}, —SR^{a}, —NR^{c}R^{d}, —NR^{c}C(═O)R^{b}, —C(═O)NR^{c}R^{d}, —C(═O)R^{a}, or —C(═O)OR^{a}.

62. The compound of any one of claims 35-61, wherein m is 0 or 1.

63. The compound of any one of claims 35-61, wherein m is 1 or 2.

64. The compound of any one of claims 35-63, wherein Ring A is aryl.

65. The compound of any one of claims 35-64, wherein Ring A is phenyl.

66. The compound of any one of claims 35-65, wherein

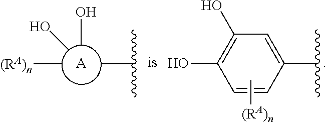

67. The compound of any one of claims 35-66, wherein each R^{A} is independently halogen or C₁-C₆ alkyl.

68. The compound of any one of claims 35-67, wherein n is 1 or 2.

69. The compound of any one of claims 35-68, wherein R² is C₁-C₆ alkyl, C₁-C₆ hydroxyalkyl, aryl, or heteroaryl; wherein the alkyl, aryl, and heteroaryl is optionally substituted with one, two, or three R^{2′}.

70. The compound of any one of claims 35-69, wherein R² is aryl or heteroaryl; wherein the aryl and heteroaryl is optionally substituted with one, two, or three R^{2′}.

71. The compound of any one of claims 35-70, wherein R² is aryl optionally substituted with one, two, or three R^{2′}.

72. The compound of any one of claims 35-70, wherein R² is heteroaryl optionally substituted with one, two, or three R^{2′}.

73. The compound of any one of claims 35-72, wherein each R^{2′} is independently C₁-C₆ alkyl, halogen, —CN, —OR^{a}, —NR^{c}R^{d}, or —C(═O)OR^{a}.

74. The compound of any one of claims 35-72, wherein each R^{2′} is independently halogen, —OR^{a}, —NR^{c}R^{d}, or —C(═O)OR^{a}.

75. The compound of any one of claims 35-74, wherein Z is hydrogen.

76. The compound of any one of claims 35-74, wherein Z is R⁶¹; and R⁶¹ is optionally substituted alkyl.

77. The compound of any one of claims 35-74, wherein Z is —R⁶⁰OC(═O)R⁶¹ or —R⁶⁰OC(═O)OR⁶¹; R⁶¹ is —CH₂— or —CH(CH₃)—; and R⁶¹ is optionally substituted alkyl, optionally substituted cycloalkyl, or optionally substituted heterocycloalkyl.

78. A compound selected from a compound of table 1, or a pharmaceutically acceptable salt, solvate, stereoisomer, tautomer, N-oxide, dimer, or trimer thereof.

79. A pharmaceutical composition comprising the compound of any one of claims 1-78, or a pharmaceutically acceptable salt, solvate, stereoisomer, tautomer, N-oxide, dimer, or trimer thereof, and a pharmaceutically acceptable excipient.

80. A method of treating a bacterial infection in a subject, comprising administering to the subject an effective amount of the compound of any one of claims 1-78, or a pharmaceutically acceptable salt, solvate, stereoisomer, tautomer, N-oxide, dimer, or trimer thereof, or the pharmaceutical composition of claim 79.

81. A method of inhibiting a bacterial penicillin-binding protein in a human infected with a bacterial infection, comprising contacting said bacterial penicillin-binding protein with an effective amount of the compound of any one of claims 1-78, or a pharmaceutically acceptable salt, solvate, stereoisomer, tautomer, N-oxide, dimer, or trimer thereof, or the pharmaceutical composition of claim 79.

82. The method of claim 80 or 81, wherein the bacterial infection is caused by Neisseria gonorrhoeae.

83. The method of claim 80 or 81, wherein the bacterial infection is caused by Pseudomonas aeruginosa.

84. The method of claim 80 or 81, wherein the bacterial infection is caused by Acinetobacter baumannii.

85. The method of claim 80 or 81, wherein the bacterial infection is caused by Pseudomonas aeruginosa/Acinetobacter baumannii.

86. The method of claim 80 or 81, wherein the bacterial infection is caused by a carbapenem-resistant enterobacteriaceae (CRE).
